(19) United States
(12) Patent Application Publication
(10) Pub. No.: US 2006/0223826 A1
(43) Pub. Date: Oct. 5, 2006

(54) Indole derivatives as somatostatin agonists or antagonists

(75) Inventors: Hidenori Abe, Osaka-shi, Osaka (JP); Shinichiro Matsunaga, Osaka (JP); Shiro Takekawa, Osaka (JP); Masanori Watanabe, Osaka (JP)

Correspondence Address: Mark Chao;Takeda Pharmaceuticals North America Inc, Intellectual Property Department, Suite 500 475 Half Day Road, Lincholnshire, IL, 60069, US

(73) Assignee: Takeda Pharmaceutical Company Limited Intellectual Property Department, Osaka (JP)

(21) Appl. No.: 10/534,725

(22) PCT Filed: Nov. 18, 2003
(86) PCT No.: PCT/JP03/14622

§ 371 Date: May 12, 2005

(30) Foreign Application Priority Data
Nov. 19, 2002 (JP) NO. 2002-335661
Mar. 19, 2003 (JP) NO. 2003-76435

(51) Int. Cl. A61K 31/496; A61K 31/454; C07D 403/02

(52) U.S. Cl. 514/254.09; 514/323; 546/201; 544/373

(57) ABSTRACT

The present invention provide a compound of the formula (I) wherein ring A represents an aromatic ring optionally having substituents; B, Y and Ya are the same or different and each represents a bond, etc.; R¹ and R² are the same or different and each represents a hydrogen atom, etc.; R³ represents a hydrogen atom, etc.; R⁴ and R⁵ are the same or different and each represents a hydrogen, etc.; R⁶ represents an indolyl group optionally having substituents; and Z and Za are the same or different and each represents a hydrogen atom, etc.; or a salt thereof or a prodrug thereof, having a somatostatin receptor binding inhibition activity and is useful for preventing and/or treating diseases associated with somatostatin.

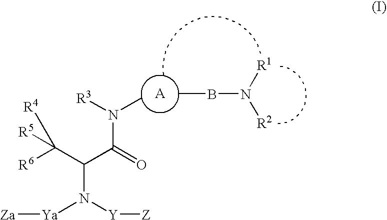

The present invention relates to novel amine compounds. In further detail, the present invention relates to a compound, which has a somatostatin receptor binding inhibition activity, and is useful for preventing and/or treating diseases associated with somatostatin.

### BACKGROUND ART

Somatostatin was found to be a growth hormone inhibiting factor (somatotropin release inhibiting factor; SRIF) in 1973.

Somatostatin receptors were found to comprise five subtypes that have been named as SSTR1, SSTR2, SSTR3, SSTR4 and SSTR5 respectively (see Endocrinology, vol. 136, pp. 3695-3697 (1995), Trends in Pharmacological Sciences, vol. 18, pp. 87-94 (1997) and Life Science, vol. 57, pp. 1249-1265 (1995)).

Somatostatin is known to inhibit production and/or secretion of various hormones, growth factors, and physiologically active substances in the living body. As the hormones inhibited by somatostatin, mentioned are growth hormone (GH), thyroid-stimulating hormones (TSH), prolactin, insulin, and glucagon. Therefore, somatostatin has various functions in endocrine systems, exocrine systems and nerve systems, and drugs targeting somatostatin are being developed (see Endocrinology, vol. 136, pp. 3695-3697 (1995) and Trends in Pharmacological Sciences, vol. 18, pp. 87-94 (1997)).

Diseases caused by somatostatin include life-style related diseases such as diabetes; central nervous system diseases, immune system diseases, and hormone-dependent tumors. Trials to develop somatostatin itself or somatostatin analogues as a drug have been conducted. For instance, octreotide known as a somatostatin receptor agonist has been marketed as a drug for treating hormone-dependent tumors.

For example, the following compounds are known as a somatostatin receptor antagonist or agonist.
1) A compound represented by the following formula: 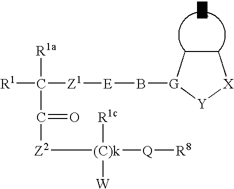
   wherein
R¹ represents a C₁₋₁₀ alkyl, aryl, aryl(C₁₋₆ alkyl), etc.; R^{1a} represents a hydrogen atom or C₁₋₃ alkyl; Z¹ represents O, CH₂, etc.; E represents SO₂, CO, etc.; B represents 1-piperidinyl having a bond at 4-position, etc.; G represents N, CH or C; Y represents C(O), etc.; X represents NR¹¹ (R¹¹ represents a hydrogen atom, C₁₋₈ alkyl, etc.), etc.; A-containing ring represents 5- to 10-membered condensed aryl, heteroaryl group containing 1 to 4 heteroatoms selected from O, S and N, etc.;
Z² represents O, CH₂, etc.; Q represents —(CH₂)x-V—(CH₂)y-(x and y independently represents 0, 1, 2, 3, 4, 5 or 6; V represents 6- to 12-membered mono- or bi-cyclic aromatic ring, etc.; R⁸ represents a hydrogen atom, etc.; R^{1c} represents a hydrogen atom, etc.; W represents a hydrogen atom, etc.; k represents 0 or 1; or a pharmaceutically acceptable salt thereof (see WO98/44921).
2) A compound represented by the following formula: 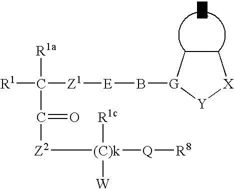
   wherein
R¹ represents a C₁₋₁₀ alkyl, aryl, aryl(C₁₋₆ alkyl), etc.; R^{1a} represents a hydrogen atom or C₁₋₃ alkyl; Z¹ represents O, CH₂, etc.; E represents SO₂, CO, etc.; B represents 1-piperidinyl having a bond at 4-position, etc.; G represents N, CH or C; Y represents C(O), etc.; X represents NR¹¹ (R¹¹ represents a hydrogen atom, C₁₋₈ alkyl, etc.), etc.; A-containing ring represents 5- to 10-membered condensed aryl, heteroaryl group lo containing 1 to 4 heteroatoms selected from O, S and N, etc.;
Z² represents O, CH₂ , etc.; Q represents —(CH₂)x-V—(CH₂)y-(x and y independently represent 0, 1, 2, 3, 4, 5 or 6; V represents a C₃₋₁₀ saturated, partially saturated or aromatic monocyclic or bi-cyclic ring containing 1 to 4 nitrogen atoms and 0 to 2 oxygen atoms or sulfur atoms, etc.; R⁸ represents a hydrogen atom, etc.; R^{1c} represents a hydrogen atom, etc.; W represents a hydrogen atom, etc.; k represents 0 or 1; or a pharmaceutically acceptable salt thereof (see WO98/45285).
3) A compound represented by the following formula: 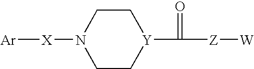
   wherein
Ar represents a C₆₋₁₀ aryl or C₁₋₉ heteroaryl; X represents a bond, etc.; Y represents N or CH; W represents —N(R²)—CH₂-Q-CH₂—N(R⁴ ) (R⁵) or —N(R²′)—CH(R³)—(CH₂)n-N(R⁴′)(R⁵′)[R²,R⁴ and R⁵ independently represents a hydrogen atom, C₁₋₆ alkyl which may be substituted by one or more of halo or trifluoromethyl group, etc.; Q represents a C₆₋₁₀ aryl, etc.; R²′, R⁴′ and R⁵′ independently represents a hydrogen atom, C₁₋₆ alkyl which may be substituted by one or more of halo or trifluoromethyl group, etc.; R³ represents, a hydrogen atom, C₁₋₆ alkyl which may be substituted by one or more of halo or trifluoromethyl group, etc.; Z represents the formula 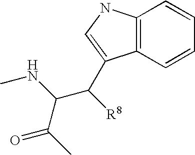
   wherein R⁸ represents a hydrogen atom or C₁₋₆ alkyl, etc.; or a pharmaceutically acceptable salt thereof (see EP-A-1086947).
4) A compound represented by the following formula: 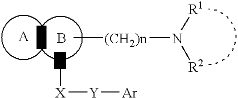
   wherein Ar represents an aromatic group optionally having substituents; X represents a methylene, S, SO, SO₂ or CO; Y represents a spacer having a main chain of 2 to 5 atoms; n represent an integer of 1 to 5;
i) R¹ and R² each represent a hydrogen atom or a lower alkyl optionally having substituents,
ii) R¹ and R², together with the adjacent nitrogen atom, form a nitrogen-containing heterocyclic ring optionally having substituents, or
iii) R¹ or R² is linked with a constituting atom on ring B together with —(CH₂)ₙ—N═ to form a spiro ring optionally having substituents; and
ring A represents an aromatic ring optionally having substituents; ring B represents 4- to 7-membered nitrogen-containing non-aromatic ring which may be further substituted by alkyl or acyl, provided that, when the ring A has a group represented by the formula —NHCOR¹¹ (R¹¹ represents alkyl group, alkoxyalkyl group, alkylthioalkyl group, cycloalkyl group, cycloalkylalkyl group, aryl group, arylalkyl group or a group represented by the formula —NHR¹² (R¹² represents alkyl group, cycloalkyl group, cycloalkyl alkyl group, aryl group or arylalkyl group)) as a substituent, X represents S, SO, SO₂ or CO; or a salt thereof (see WO99/52875).
5) A compound represented by the following formula: 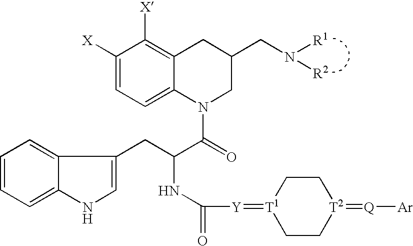
   wherein
X and X′ are the same or different and each represents a hydrogen atom, a fluorine atom, a chlorine atom or an amino optionally having substituents, and at least one of X and X′ represents a fluorine atom, a chlorine atom or an amino optionally having substituents;
R¹ and R² represent a hydrogen atom or C₁₋₆ alkyl optionally having substituents, or R¹ and R², together with the adjacent nitrogen atom, form a nitrogen-containing heterocyclic ring optionally having substituents;
Y and Q are the same or different and each represents a bond or a spacer having a main chain of 1 to 6 atoms; [symbol] represents a single bond or a double bond;
T¹ and T² are the same or different and each represents a C(R⁹) (R⁹ represents a hydrogen atom, a hydroxy or C₁₋₆ alkyl) or N, when each of the adjacent [symbol] is a single bond, and C when the adjacent [symbol] is a double bond; and
Ar represents an aromatic group optionally having substituents, a C₃₋₉ cycloalkyl group optionally having substituents, a 3 to 9-membered saturated heterocyclic group optionally having substituents, a hydrogen atom or a halogen atom; or a salt thereof (see WO01/25228).
6) A compound represented by the following formula: 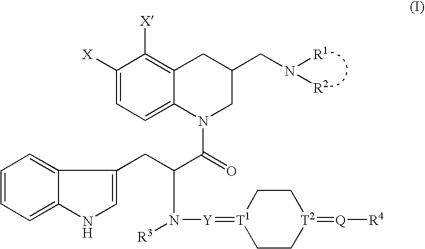
   wherein
X and X′ are the same or different and each represents a hydrogen atom, halogen atom or an amino optionally having substituents;
R¹ and R² represent a hydrogen atom or C₁₋₆ alkyl optionally having substituents, or R¹ and R², together with the adjacent nitrogen atom, form a nitrogen-containing-heterocyclic ring optionally having substituents;
Q represents a bond or a spacer having a main chain of 1 to 6 atoms;
Y represents a bond or —CH₂—Y′— (Y′ represents a bond or a spacer having a main chain of 1 to 5 atoms);
[symbol] represents a single bond or a double bond;
when each of adjacent [symbol] is a single bond, T¹ and T² are the same or different, they represent C(R⁵) (R⁵ represents a hydrogen atom, a hydroxy or C₁₋₆ alkyl) or N and when each of the adjacent [symbol] is a double bond, T¹ and T² represent C;
R³ represents a hydrogen atom, optionally halogenated C₁₋₆ alkyl, optionally halogenated C₁₋₆ alkyl-carbonyl or optionally halogenated C₁₋₆ alkylsulfonyl; and
R⁴ represents an aromatic group optionally having substituents, a C₃₋₉ cycloalkyl group optionally having substituents, a 3 to 9-membered saturated heterocyclic ring optionally having substituents, a hydrogen atom or halogen atom; or a salt thereof (see WO02/16350).

### DISCLOSURE OF THE INVENTION

It is desired to develop a compound which has excellent somatostatin receptor binding inhibition activity, etc., as well as superior properties as a pharmaceutical product, such as oral absorbability, pharmacokinetics and the like.

The present invention relates to:
[1] a compound represented by the formula: 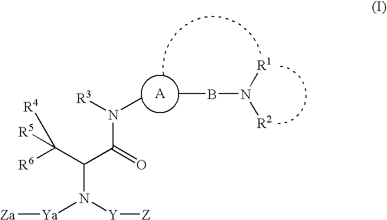
   wherein
ring A represents an aromatic ring optionally having substituents;
B, Y and Ya are the same or different and each represents a bond or a spacer having a main chain of 1 to 6 atoms;
R¹ and R² are the same or different and each represents a hydrogen atom, hydrocarbon group optionally having substituents or heterocyclic group optionally having substituents, or R¹ and R², together with the adjacent nitrogen atom, form a nitrogen-containing heterocyclic ring optionally having substituents, or R¹ is linked with ring A together with the adjacent nitrogen atom and B to form a 5- to 7-membered nitrogen-containing heterocyclic ring;
R³ represents a hydrogen atom, hydrocarbon group optionally having substituents or heterocyclic group optionally having substituents;
R⁴ and R⁵ are the same or different and each represents a hydrogen atom or hydrocarbon group optionally having substituents, or R⁴ and R⁵, together with the adjacent carbon atom, form a ring optionally having substituents;
R⁶ represents an indolyl group optionally having substituents; and
Z and Za are the same or different and each represents a hydrogen atom, a halogen atom or a cyclic group optionally having substituents; or a salt thereof [hereinafter sometimes to be abbreviated as compound (I)];
[2] a prodrug of the compound (I);
[3] the compound (I) wherein R³ is a hydrogen atom or a C₁₋₆ alkyl optionally having substituents;
[4] the compound (I) wherein one of R⁴ and R⁵ is a hydrogen atom, and the other is a C₁₋₆ alkyl optionally having substituents;
[5] the compound (I) wherein Z is a cyclic group optionally having substituents;
[6] the compound (I) according to the above [5] wherein the cyclic group is piperidinyl or piperazinyl;
[7] the compound (I) according to the above [5] wherein Z is piperidinyl or piperazinyl, each of which is substituted by a group of the formula: -Yd-Ara wherein Yd represents a bond or a spacer having a main chain of 1 to 6 atoms, and Ara represents a monocyclic group optionally having substituents;
[8] the compound (I) wherein Ya is a bond, and Za is a hydrogen atom;
[9] the compound (I) wherein B is a C₁₋₆ alkylene;
[10] the compound (I) wherein the aromatic ring represented by ring A is benzene;
[11] the compound (I) wherein R³ and R² are C₁₋₆ alkyl;
[12] the compound (I) wherein Y is —CO—;
[13] the compound (I) which is

N-((1R,2S)-1-(((5-((dimethylamino)methyl)-2-((methylamino)carbonyl)phenyl)amino)carbonyl)-2-(1H-indol-3-yl)propyl)-4-(2-methylphenyl)-1-piperidinecarboxamide;

N-((1R,2S)-1-(((2-((dimethylamino)carbonyl)-5-((dimethylamino)methyl)phenyl)amino)carbonyl)-2-(1H-indol-3-yl)propyl)-4-(4-fluorophenyl)-1-piperidinecarboxamide;

N-((1R,2S)-1-(((5-((dimethylamino)methyl)-2-methoxyphenyl)amino)carbonyl)-2-(1H-indol-3-yl)propyl)-4-(4-fluoro-2-methylphenyl)-3-oxo-1-piperazinecarboxamide;

N-((1R,2S)-1-(((5-((dimethylamino)methyl)-2-methoxyphenyl)-amino)carbonyl)-2-(1H-indol-3-yl)propyl)-4-(2-methylphenyl)-1-piperazinecarboxamide;

N-((1R,2S)-1-(((5-((dimethylamino)methyl)-2-ethoxyphenyl)amino)carbonyl)-2-(1H-indol-3-yl)propyl)-4-(4-fluorophenyl)-1-piperazinecarboxamide; or

N-((1R,2S)-1-(((5-((dimethylamino)methyl)-2-ethoxyphenyl)amino)carbonyl)-2-(1H-indol-3-yl)propyl)-4-phenyl-1-piperidinecarboxamide;
[14] a pharmaceutical preparation comprising the compound (I) or a prodrug thereof;
[15] the pharmaceutical preparation according to item [14], which is a somatostatin receptor binding inhibitor;
[16] the pharmaceutical preparation according to item [15], which is a somatostatin subtype 2 receptor binding inhibitor;
[17] the pharmaceutical preparation according to item [14], which is a somatostatin receptor agonist;
[18] the pharmaceutical preparation according to item [17], which is a somatostatin subtype 2 receptor agonist;
[19] the pharmaceutical preparation according to item [14], which is a prophylactic or therapeutic agent for diabetes or diabetic complications;
[20] the pharmaceutical preparation according to item [14], which is a prophylactic or therapeutic agent for obesity;
[21] use of the compound (I) or a prodrug thereof for manufacturing a somatostatin receptor binding inhibitor;
[22] a method for inhibiting somatostatin receptor binding in a mammal, which comprises administering to the mammal an effective amount of the compound (I) or a prodrug thereof;
[23] use of the compound (I) or a prodrug thereof for manufacturing a prophylactic or therapeutic agent for diabetes or diabetic complications;
[24] a method for preventing or treating diabetes or diabetic complications in a mammal, which comprises administering to the mammal an effective amount of the compound (I) or a prodrug thereof;
[25] use of the compound (I) or a prodrug thereof for manufacturing a prophylactic or therapeutic agent for obesity;
[26], a method for preventing or treating obesity in a mammal, which comprises administering to the mammal an effective amount of the compound (I) or a prodrug thereof;
[27] a method for producing a compound (I), which comprises reacting a compound of the formula: 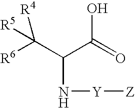
   wherein
Y represents a bond or a spacer having a main chain of 1 to 6 atoms;
R⁴ and R⁵ are the same or different, and each represents a hydrogen atom or a hydrocarbon group optionally having substituents, or R⁴ and R⁵, together with the adjacent carbon atom, form a ring optionally having substituents;
R⁶ represents an indolyl group optionally having substituents; Z represents a hydrogen atom, a halogen atom or a cyclic group optionally having substituents; or a salt thereof with a compound of the formula: 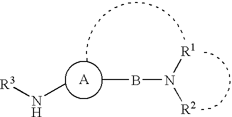
   wherein
ring A represents an aromatic ring optionally having substituents;
B represents a bond or a spacer having a main chain of 1 to 6 atoms;
R¹ and R² are the same or different, and each represents a hydrogen atom, a hydrocarbon group optionally having substituents or a heterocyclic group optionally having substituents, or R¹ and R², together with the adjacent nitrogen atom, form a nitrogen-containing heterocyclic ring optionally having substituents, or R¹ is linked with ring A together with the adjacent nitrogen atom and B to form a 5- to 7-membered nitrogen-containing heterocyclic ring;
R³ represents a hydrogen atom, a hydrocarbon group optionally having substituents or a heterocyclic group optionally having substituents; or a salt thereof to give a compound of the formula: 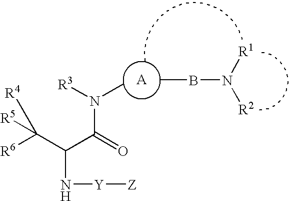
   wherein each symbol is as defined above; or a salt thereof, and optionally reacting the compound or a salt thereof with a compound of the formula: L⁴-Ya-Za wherein L⁴ represents a leaving group; Ya represents a bond or a spacer having a main chain of 1 to 6 atoms; Za represents a hydrogen atom, a halogen atom or a cyclic group optionally having substituents; or a salt thereof;
[28] a compound of the formula: 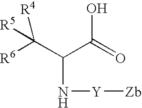
   wherein
Y represents a bond or a spacer having a main chain of 1 to 6 atoms;
R⁴ and R⁵ are the same or different, and each represents a hydrogen atom or a hydrocarbon group optionally having substituents, or R⁴ and R⁵, together with the adjacent carbon atom, form a ring optionally having substituents;
R⁶ represents an indolyl group optionally having substituents; Zb represents-piperidinyl or piperazinyl, each of which is substituted by a group of the formula: -Yd-Ara wherein Yd represents a bond or a spacer having a main chain of 1 to 6 atoms, and Ara represents a monocyclic group optionally having substituents; or a salt thereof.

The definition of each symbol of the formula (I) is described in detail in the following.

The “aromatic ring” in the “aromatic ring optionally having substituents” represented by ring A includes, for example, aromatic hydrocarbon, aromatic heterocyclic ring, etc.

The aromatic hydrocarbon includes, for example, C₆₋₁₄ aromatic hydrocarbon. The preferable examples of the aromatic hydrocarbon include benzene, naphthalene, indene, fluorene, anthracene, etc.

The aromatic heterocyclic ring includes, for example, 5- or 6-membered aromatic heterocyclic ring, fused polycyclic aromatic heterocyclic ring, etc.

Said “5- or 6-membered aromatic heterocyclic ring” includes, for example, 5- or 6-membered aromatic heterocyclic ring containing, in addition to carbon atoms, 1 to 4 (preferably 1 to 3) heteroatoms selected from nitrogen, sulfur and oxygen atoms, etc. The preferable examples of “5- or 6-membered aromatic heterocyclic ring” include thiophene, furan, pyrrole, imidazole, pyrazole, thiazole, isothiazole, oxazole, isoxazole, pyridine, pyrazine, pyrimidine, pyridazine, 1,2,4-oxadiazole, 1,3,4-oxadiazole, 1,2,4-thiadiazole, 1,3,4-thiadiazole, furazan, tetrazole, etc.

The “fused polycyclic aromatic heterocyclic ring” includes, for example, 9- to 14-membered (preferably 9- or 10-membered) fused polycyclic (preferably bi- to tetra-cyclic, more preferably bi- or tri-cyclic) aromatic heterocyclic ring containing, in addition to carbon atoms, 1 to 4 heteroatoms selected from nitrogen, sulfur and oxygen atoms, etc. The preferable examples of the “fused polycyclic aromatic heterocyclic ring” include benzofuran, benzothiophene, benzimidazole, benzoxazole, benzothiazole, benzisothiazole, naphtho[2,3-b]thiophene, isoquinoline, quinoline, indole, quinoxaline, phenanthridine, phenothiazine, phenoxazine, phthalazine, naphthyridine, quinazoline, cinnoline, carbazole, β-carboline, acridine, phenazine, etc.

The “aromatic ring” in the “aromatic ring optionally having substituents” represented by ring A is preferably a C₆₋₁₄ aromatic hydrocarbon or a 5- or 6-membered aromatic heterocyclic ring. Of these, benzene and thiazole are preferable, and benzene is particularly preferable.

Ring A may have substituents in addition to a group represented by the formula 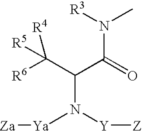
wherein each symbol is as defined above, and a group represented by the formula 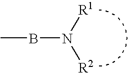
wherein each symbol is as defined above. Such “substituent” includes, for example, halogen atoms (e.g., fluorine, chlorine, bromine, iodine), nitro, cyano, hydroxy, hydrocarbon group optionally having substituents, optionally halogenated C₁₋₆ alkoxy, optionally halogenated C₁₋₆ alkylthio, C₆₋₁₄ aryloxy optionally having substituents, C₇₋₁₉ aralkyloxy optionally having substituents, amino, mono- or di-C₁₋₆ alkylamino (e.g., methylamino, ethylamino, propylamino, isopropylamino, butylamino, dimethylamino, diethylamino, dipropylamino, dibutylamino, ethylmethylamino), 5- to 7-membered heterocyclic group optionally having substituents, acyl, acylamino, acyloxy, C₁₋₆ alkoxy-C₁₋₆ alkoxy, C₃₋₈ cycloalkyl-C₁₋₆ alkoxy, etc.

The “hydrocarbon group” in the “hydrocarbon group optionally having substituents” includes, for example, alkyl, alkenyl, alkynyl, cycloalkyl, cycloalkenyl, aryl, aralkyl, etc. Among them, the following hydrocarbon group having 1 to 19 carbon atoms, etc. are preferable:
a) C₁₋₆ alkyl (e.g., methyl, ethyl, propyl, isopropyl, butyl, isobutyl, sec-butyl, tert-butyl, pentyl, hexyl);
b) C₂₋₆ alkenyl (e.g., vinyl, allyl, isopropenyl, 2-butenyl);
c) C₂₋₆ alkynyl (e.g., ethynyl, propargyl, 2-butynyl);
d) C₃₋₈ cycloalkyl which may be condensed with benzene ring (e.g., cyclopropyl, cyclobutyl, cyclopentyl, cyclohexyl, cycloheptyl, cyclooctyl, dihydroindenyl);
e) C₃₋₈ cycloalkenyl which may be condensed with benzene ring (e.g., cyclopropenyl, cyclobutenyl, cyclopentenyl, cyclohexenyl, cycloheptenyl, cyclooctenyl);
f) C₆₋₁₄ aryl (e.g., phenyl, 1-naphthyl, 2-naphthyl, 2-indenyl, 2-anthryl), preferably phenyl;
g) C₇₋₁₉ aralkyl (e.g., benzyl, phenethyl, diphenylmethyl, triphenylmethyl, 1-naphthylmethyl, 2-naphthylmethyl, 2,2-diphenylethyl, 3-phenylpropyl, 4-phenylbutyl, 5-phenylpentyl), preferably benzyl.

The “substituent” of the “hydrocarbon group optionally having substituents” includes, for example, halogen atoms (e.g., fluorine, chlorine, bromine, iodine), C₁₋₃ alkylenedioxy (e.g., methylenedioxy, ethylenedioxy), nitro, cyano, optionally halogenated C₁₋₆ alkoxy, optionally halogenated C₁₋₆ alkylthio, hydroxy, amino, mono- or di-C₁₋₆ alkylamino (e.g., methylamino, ethylamino, propylamino, isopropylamino, butylamino, dimethylamino, diethylamino, dipropylamino, dibutylamino, ethylmethylamino), 5- to 7-membered heterocyclic group optionally having substituents, formyl, carboxy, carbamoyl, thiocarbamoyl, optionally halogenated C₁₋₆ alkyl-carbonyl, C₁₋₆ alkoxy-carbonyl, C₆₋₁₄ aryl-carbonyl (e.g., benzoyl, 1-naphthoyl, 2-naphthoyl), heterocyclic carbonyl optionally having substituents, C₆₋₁₄ aryloxy-carbonyl (e.g., phenyloxycarbonyl, 1-naphthyloxycarbonyl, 2-naphthyloxycarbonyl), C₇₋₁₉ aralkyloxy-carbonyl (e.g., benzyloxycarbonyl, phenethyloxycarbonyl, diphenylmethyloxycarbonyl, triphenylmethyloxycarbonyl, 1-naphthylmethyloxycarbonyl, 2-naphthylmethyloxycarbonyl, 2,2-diphenylethyloxycarbonyl, 3-phenylpropyloxycarbonyl, 4-phenylbutyloxycarbonyl, 5-phenylpentyloxycarbonyl), mono- or di-C₁-6 alkyl-carbamoyl (e.g., methylcarbamoyl, ethylcarbamoyl, dimethylcarbamoyl, diethylcarbamoyl, ethylmethylcarbamoyl), C₆₋₁₄ aryl-carbamoyl (e.g., phenylcarbamoyl), heterocyclic carbamoyl optionally having substituents, optionally halogenated C₁₋₆ alkylsulfonyl, C₆₋₁₄ arylsulfonyl (e.g., phenylsulfonyl, 1-naphthylsulfonyl, 2-naphthylsulfonyl), formylamino, optionally halogenated C₁₋₆ alkyl-carboxamide, C₆₋₁₄ aryl-carboxamide (e.g., phenylcarboxamide, naphthylcarboxamide), C₁₋₆ alkoxy-carboxamide (e.g., methoxycarboxamide, ethoxycarboxamide, propoxycarboxamide, butoxycarboxamide), C₁₋₆ alkylsulfonylamino (e.g., methylsulfonylamino, ethylsulfonylamino), C₁₋₆ alkyl-carbonyloxy (e.g., acetoxy, propanoyloxy), C₆₋₁₄ aryl-carbonyloxy (e.g., benzoyloxy, 1-naphthoyloxy, 2-naphthoyloxy), C₁₋₆ alkoxy-carbonyloxy (e.g., methoxycarbonyloxy, ethoxycarbonyloxy, propoxycarbonyloxy, butoxycarbonyloxy), mono- or di-C₁₋₆ alkyl-carbamoyloxy (e.g., methylcarbamoyloxy, ethylcarbamoyloxy, dimethylcarbamoyloxy, diethylcarbamoyloxy), C₆₋₁₄ aryl-carbamoyloxy (e.g., phenylcarbamoyloxy, naphthylcarbamoyloxy), 5- or 6-membered heterocyclic carbonyloxy (e.g., nicotinoyloxy), C₆₋₁₄ aryloxy (e.g., phenoxy, naphthoxy), etc. The number of the substituents is, for example, 1 to 5, preferably 1 to 3. When the number of the substituents is 2 or more, these substituents may be the same or different.

The above-mentioned “optionally halogenated C₁₋₆ alkoxy” includes, for example, C₁₋₆ alkoxy (e.g., methoxy, ethoxy, propoxy, butoxy, pentyloxy) which may have 1 to 5, preferably 1 to 3 halogen atoms (e.g., fluorine, chlorine, bromine, iodine), etc. Concrete examples are methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy, hexyloxy, etc.

The above-mentioned “optionally halogenated C₁₋₆ alkylthio” includes; for example, C₁₋₆ alkylthio (e.g., methylthio, ethylthio, propylthio, isopropylthio, butylthio, sec-butylthio, tert-butylthio) which may have 1 to 5, preferably 1 to 3 halogen atoms (e.g., fluorine, chlorine, bromine, iodine), etc. Concrete examples are methylthio, difluoromethylthio, trifluoromethylthio, ethylthio, propylthio, isopropylthio, butylthio, 4,4,4-trifluorobutylthio, pentylthio, hexylthio, etc.

The “5- to 7-membered heterocyclic group” in the “5- to 7-membered heterocyclic group optionally having substituents” includes, for example, 5- to 7-membered heterocyclic group containing, in addition to carbon atoms, 1 to 4 heteroatoms selected from nitrogen, sulfur and oxygen atoms.

The preferable examples of the 5- to 7-membered heterocyclic group include, a 5- to 7-membered-non-aromatic heterocyclic group such as pyrrolidinyl (e.g., 1-, 2- or 3-pyrrolidinyl); imidazolidinyl (e.g., 1-, 2-, 4- or 5-imidazolidinyl); imidazolinyl (e.g., 2- or 4-imidazolinyl); pyrazolidinyl (e.g., 2-, 3- or 4-pyrazolidinyl); piperidinyl (e.g., 1-, 2-, 3- or 4-piperidinyl); piperazinyl (e.g., 1- or 2-piperazinyl); morpholinyl; thiomorpholinyl, etc.; and

a 5- to 7-membered aromatic heterocyclic group such as thienyl (e.g., 2- or 3-thienyl); furyl (e.g., 2- or 3-furyl);

pyrrolyl (e.g., 1-, 2- or 3-pyrrolyl); imidazolyl (e.g., 1-, 2- or 4-imidazolyl); thiazolyl (e.g., 2-, 4- or 5-thiazolyl); oxazolyl (e.g., 2-, 4- or 5-oxazolyl); isothiazolyl (e.g., 3-isothiazolyl); isoxazolyl (e.g., 3-isoxazolyl); pyridyl (e.g., 2-, 3- or 4-pyridyl); pyrazolyl (e.g., 1-, 3- or 4-pyrazolyl); pyrazinyl (e.g., 2-pyrazinyl); pyrimidinyl (e.g., 2-, 4- or 5-pyrimidinyl); pyridazinyl (e.g., 3- or 4-pyridazinyl); oxadiazolyl (e.g., 1,2,4-oxadiazol-5-yl; 1,2,4-oxadiazol-3-yl); thiadiazolyl (e.g., 1,2,4-thiadiazol-5-yl; 1,2,4-thiadiazol-3-yl); triazolyl (e.g., 1,2,3-triazol-1-yl; 1,2,3-triazol-4-yl; 1,2,4-triazol-1-yl; 1,2,4-triazol-3-yl); tetrazolyl (e.g., 1- or 5-tetrazolyl), etc.

The above-mentioned “optionally halogenated C₁₋₆ alkyl-carbonyl” includes, for example, C₁₋₆ alkyl-carbonyl (e.g., acetyl, propanoyl, butanoyl, pentanoyl, hexanoyl) which may have 1 to 5, preferably 1 to 3 halogen atoms (e.g., fluorine, chlorine, bromine, iodine), etc. Concrete examples are acetyl, monochloroacetyl, trifluoroacetyl, trichloroacetyl, propanoyl, butanoyl, pentanoyl, hexanoyl, etc.

Said “C₁₋₆ alkoxy-carbonyl” includes, for example, methoxycarbonyl, ethoxycarbonyl, propoxycarbonyl, tert-butoxycarbonyl, etc.

The “heterocyclic carbonyl” in the “heterocyclic carbonyl optionally having substituents” includes, for example, nicotinoyl, isonicotinoyl, 2-thenoyl, 3-thenoyl, 2-furoyl, 3-furoyl, morpholinocarbonyl, piperidinocarbonyl, pyrrolidin-1-ylcarbonyl, indolylcarbonyl, etc.

The “heterocyclic carbamoyl” in the “heterocyclic carbamoyl optionally having substituents” includes, for example, morpholinocarbamoyl, piperidinocarbamoyl, 2-pyridylcarbamoyl, 3-pyridylcarbamoyl, 4-pyridylcarbamoyl, 2-thienylcarbamoyl, 3-thienylcarbamoyl, indolylcarbamoyl, etc.

The above-mentioned “optionally halogenated C₁₋₆ alkyl-sulfonyl” includes, for example, C₁₋₆ alkyl-sulfonyl (e.g., methylsulfonyl, ethylsulfonyl, propylsulfonyl, isopropylsulfonyl, butylsulfonyl, sec-butylsulfonyl, tert-butylsulfonyl) which may have 1 to 5, preferably 1 to 3 halogen atoms (e.g., fluorine, chlorine, bromine, iodine), etc. Concrete examples are methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl, hexylsulfonyl, etc.

The above-mentioned “optionally halogenated C₁₋₆ alkyl-carboxamide” includes, for example, C₁₋₆ alkyl-carboxamide (e.g., acetamide, propanamide, butanamide) which may have 1 to 5, preferably 1 to 3 halogen atoms (e.g., fluorine, chlorine, bromine, iodine), etc. Concrete examples are acetamide, trifluoroacetamide, propanamide, and butanamide.

The substituent in the “5- to 7-membered heterocyclic group optionally having substituents”, “heterocyclic carbonyl optionally having substituents” and “heterocyclic carbamoyl optionally having substituents” includes, for example, halogen atoms (e.g., fluorine, chlorine, bromine, iodine), C₁₋₃ alkylenedioxy (e.g., methylenedioxy, ethylenedioxy), nitro, cyano, optionally halogenated C₁₋₆ alkyl, optionally halogenated C₃₋₆ cycloalkyl, optionally halogenated C₁₋₆ alkoxy, optionally halogenated C₁₋₆ alkylthio, hydroxy, amino, mono- or di-C₁₋₆ alkylamino (e.g., methylamino, ethylamino, propylamino, isopropylamino, butylamino, dimethylamino, diethylamino, dipropylamino, dibutylamino, ethylmethylamino), formyl, carboxy, carbamoyl, thiocarbamoyl, optionally halogenated C₁₋₆ alkyl-carbonyl, C₁₋₆ alkoxy-carbonyl, mono- or di-C₁₋₆ alkyl-carbamoyl (e.g., methylcarbamoyl, ethylcarbamoyl, dimethylcarbamoyl, diethylcarbamoyl, ethylmethylcarbamoyl), optionally halogenated C₁₋₆ alkylsulfonyl, sulfamoyl, formylamino, optionally halogenated C₁₋₆ alkyl-carboxamide, C₁₋₆ alkoxy-carboxamide (e.g., methoxycarboxamide, ethoxycarboxamide, propoxycarboxamide, butoxycarboxamide), C₁₋₆ alkylsulfonylamino (e.g., methylsulfonylamino, ethylsulfonylamino), C₁₋₆ alkyl-carbonyloxy (e.g., acetoxy, propanoyloxy), C₁₋₆ alkoxy-carbonyloxy (e.g., methoxycarbonyloxy, ethoxycarbonyloxy, propoxycarbonyloxy, butoxycarbonyloxy), mono- or di-C₁₋₆ alkyl-carbamoyloxy (e.g., methylcarbamoyloxy, ethylcarbamoyloxy, dimethylcarbamoyloxy, diethylcarbamoyloxy), 5- or 6-membered aromatic heterocyclic group (e.g., tetrazolyl, thiazolyl, oxazolyl), hydroxy-C₁₋₆ alkyl (e.g., hydroxymethyl), etc. The number of the substituents is, for example, 1 to 3. When the number of the substituents is 2 or more, these substituents may be the same or different.

The above “optionally halogenated C₁₋₆ alkyl” includes, for example, C₁₋₆ alkyl (e.g., methyl, ethyl, propyl, isopropyl, butyl, isobutyl, sec-butyl, tert-butyl, pentyl, hexyl) which may have 1 to 5, preferably 1 to 3 halogen atoms (e.g., fluorine, chlorine, bromine, iodine). Concrete examples are methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl, 6,6,6-trifluorohexyl, etc.

The above-mentioned “optionally halogenated C₃₋₆ cycloalkyl” includes, for example, a C₃₋₆ cycloalkyl (e.g., cyclopropyl, cyclobutyl, cyclopentyl, cyclohexyl) which may have 1 to 5, preferably 1 to 3 halogen atoms (e.g., fluorine, chlorine, bromine, iodine), etc. Concrete examples are cyclopropyl, cyclobutyl, cyclopentyl, cyclohexyl, 4,4-dichlorocyclohexyl, 2,2,3,3-tetrafluorocyclopentyl, 4-chlorocyclohexyl, etc.

The “optionally halogenated C₁₋₆ alkoxy”, “optionally halogenated C₁₋₆ alkylthio”, “optionally halogenated C₁₋₆ alkyl-carbonyl”, “C₁₋₆ alkoxy-carbonyl”, “optionally halogenated C₁₋₆ alkylsulfonyl” and “optionally halogenated C₁₋₆ alkyl-carboxamide” are exemplified by those mentioned as the “substituent” of the above “hydrocarbon group optionally having substituents”.

The “optionally halogenated C₁₋₆ alkoxy”, “optionally halogenated C₁₋₆ alkylthio” and “5- to 7-membered heterocyclic group optionally having substituents”, which are “substituent” of the ring A, are exemplified by those mentioned as the “substituent” of the above “hydrocarbon group optionally having substituents”.

The “C₆₋₁₄ aryloxy” in the “C₆₋₁₄ aryloxy optionally having substituents” which is a “substituent” of the ring A includes, for example, phenyloxy, 1-naphthyloxy, 2-naphthyloxy, etc.

The “C₇₋₁₉ aralkyloxy” in the “C₇₋₁₉ aralkyloxy optionally having substituents” which is a “substituent” of the ring A includes, for example, benzyloxy, phenethyloxy, diphenylmethyloxy, triphenylmethyloxy, 1-naphthylmethyloxy, 2-naphthylmethyloxy, 2,2-diphenylethyloxy, 3-phenylpropyloxy, 4-phenylbutyloxy, 5-phenylpentyloxy, etc.

The substituent in the “C₆₋₁₄ aryloxy optionally having substituents” and “C₇₋₁₉ aralkyloxy optionally having substituents” are exemplified by those mentioned as the substituent in the above “5- to 7-membered heterocyclic group optionally having substituents”. The number of the substituents is, for example, 1 to 3. When the number of the substituents is 2 or more, these substituents may be the same or different.

The “acyl” which is a “substituent” of the ring A includes, for example, an acyl represented by the formula: —CO—R⁷, —CO—OR⁷, —CO—NR⁷R⁸, —CS—NR⁷R⁸, —SO₂—R^{7a}—SO—R , —SO₂—NR⁷R⁸ wherein R⁷ represents (i) a hydrogen atom, (ii) a hydrocarbon group optionally having substituents, or (iii) a heterocyclic group optionally having substituents; R^{7a} represents (i) a hydrocarbon group optionally having substituents, or (ii) a heterocyclic group optionally having substituents; R⁸ represents a hydrogen atom or C₁₋₆ alkyl; R⁷ and R⁸, together with the adjacent nitrogen atom, may form a nitrogen-containing heterocyclic ring optionally having substituents, etc.

The “hydrocarbon group optionally having substituents” represented by R⁷ or R^{7a} is exemplified by those mentioned as the “substituent” of the above ring A.

The “heterocyclic group” in the “heterocyclic group optionally having substituents” represented by R⁷ or R^{7a} includes, for example, 4- to 14-membered mono-, bi- or tri-cyclic (i) aromatic heterocyclic group or (ii) non-aromatic heterocyclic group, or (iii) 7- to 10-membered bridged heterocyclic group, containing, in addition to carbon atoms, 1 to 4 heteroatoms selected from nitrogen, sulfur and oxygen atoms, etc.

The “aromatic heterocyclic group” includes, for example, 4- to 14-membered, preferably 4- to 10-membered aromatic heterocyclic groups containing, in addition to carbon atoms, 1 to 4 heteroatoms selected from the group consisting of nitrogen, sulfur and oxygen atoms, etc. The preferable examples of “aromatic heterocyclic group” include a monocyclic aromatic heterocyclic group such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, furazanyl, etc.;

a fused polycyclic (preferably bi- or tri-cyclic) aromatic heterocyclic group such as benzothiophenyl, benzofuranyl, benzimidazolyl, benzoxazolyl, benzothiazolyl, benzisothiazolyl, naphtho[2,3-b]thiophenyl, phenoxathiinyl, indolyl, isoindolyl, 1H-indazolyl, purinyl, 4H-quinolidinyl, isoquinolinyl, quinolinyl, phthalazinyl, naphthylidinyl, quinoxalinyl, quinazolinyl, cinnolinyl, carbazolyl, β-carbolinyl, phenanthridinyl, acridinyl, phenazinyl, phenothiazinyl, phenoxazinyl, phthalimide, etc.

The “non-aromatic heterocyclic group” includes, for example, 4- to 14-membered (preferably 4- to 10-membered) non-aromatic heterocyclic group containing, in addition to carbon atoms, 1 to 4 heteroatoms selected from nitrogen, sulfur and oxygen atoms, etc. The preferable examples of the “non-aromatic heterocyclic groupf” include a monocyclic non-aromatic heterocyclic group such as azetidinyl, tetrahydrothiophenyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydrothiazolyl, tetrahydroisothiazolyl, tetrahydrooxazolyl, tetrahydroisoxazolyl, piperidinyl (including oxopiperidinyl and dioxopiperidinyl), piperazinyl (including oxopiperazinyl and dioxopiperazinyl), tetrahydropyridinyl, dihydropyridinyl, tetrahydropyrimidinyl, tetrahydropyridazinyl, azepanyl, morpholinyl, thiomorpholinyl, diazepanyl, etc.;

a fused polycyclic (preferably bi- or tri-cyclic) non-aromatic heterocyclic group such as dihydrobenzofuranyl, dihydrobenzimidazolyl, dihydrobenzoxazolyl, dihydrobenzothiazolyl, dihydrobenzisothiazolyl, dihydronaphtho[2,3-b]thiophenyl, tetrahydroisoquinolinyl, tetrahydroquinolinyl, indolinyl, isoindolinyl, tetrahydrothieno([2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthylidinyl, tetrahydroquinazolinyl, tetrahydrocinnolinyl, tetrahydrocarbazolyl, tetrahydro-β-carbolinyl, tetrahydroacridinyl, tetrahydrophenazinyl, tetrahydrothioxanthenyl, octahydroisoquinolinyl, etc.

The preferable examples of the “7- to 10-membered bridged heterocyclic group” include, for example, quinuclidinyl, 7-azabicyclo[2.2.1]heptanyl, etc.

The “substituent” of the “heterocyclic group optionally having substituents” is exemplified by those mentioned as the substituent in the “5- to 7-membered heterocyclic group optionally having substituents”. The number of the substituents is, for example, 1 to 3. When the number of the substituents is 2 or more, these substituents may be the same or different.

The “C₁₋₆ alkyl” represented by R⁸ includes, for example, methyl, ethyl, propyl, isopropyl, butyl, isobutyl, sec-butyl, tert-butyl, pentyl, hexyl, etc.

The “nitrogen-containing heterocyclic ring” in the “nitrogen-containing heterocyclic ring optionally having substituents” formed by R⁷ and R⁸ together with the adjacent nitrogen atom includes, for example, the 3- to 8-membered nitrogen-containing heterocyclic rings containing at least one nitrogen atom in addition to carbon atoms and optionally further containing 1 to 3 heteroatoms selected from nitrogen, sulfur and oxygen atoms. Concrete examples are aziridine, azetidine, morpholine, thiomorpholine, piperidine, piperazine, pyrrolidine, azepane, azocane, hexahydropyrimidine, 1,4-diazepane; and unsaturated cyclic amines thereof (e.g., 1,2,5,6-tetrahydropyridine, etc.), etc. Of these, preferred are morpholine, piperidine, piperazine, pyrrolidine, etc.

The “substituent” of the “nitrogen-containing heterocyclic ring optionally having substituents” is exemplified by those mentioned as the “substituent” of the above “5- to 7-membered heterocyclic group optionally having substituents”. The number of the substituents is, for example, 1 to 3. When the number of the substituents is 2 or more, these substituents may be the same or different.

The “acyl” is preferably formyl, carboxy, carbamoyl, optionally halogenated C₁₋₆ alkyl-carbonyl (e.g., acetyl), C₁₋₆ alkoxy-carbonyl (e.g., methoxycarbonyl, ethoxycarbonyl, propoxycarbonyl, tert-butoxycarbonyl), C₆₋₁₄ aryl-carbonyl optionally having substituents (e.g., benzoyl, 1-naphthoyl, 2-naphthoyl), C₆₋₁₄ aryloxy-carbonyl optionally having substituents (e.g., phenoxycarbonyl), C₇₋₁₉ aralkyloxy-carbonyl optionally having substituents (e.g., benzyloxycarbonyl, phenethyloxycarbonyl), 5- or 6-membered heterocyclic carbonyl optionally having substituents (e.g., nicotinoyl, isonicotinoyl, 2-thenoyl, 3-thenoyl, 2-furoyl, 3-furoyl, morpholinocarbonyl, piperidinocarbonyl, pyrrolidin-1-ylcarbonyl), mono- or di-C₁₋₆ alkyl-carbamoyl (e.g., methylcarbamoyl, ethylcarbamoyl, dimethylcarbamoyl, diethylcarbamoyl, ethylmethylcarbamoyl), C₆₋₁₄ aryl-carbamoyl optionally having substituents (e.g., phenylcarbamoyl, 1-naphthylcarbamoyl, 2-naphthylcarbamoyl), 5- or 6-membered heterocyclic carbamoyl optionally having substituents (e.g., 2-pyridylcarbamoyl, 3-pyridylcarbamoyl, 4-pyridylcarbamoyl, 2-thienylcarbamoyl, 3-thienylcarbamoyl), optionally halogenated C₁₋₆ alkylsulfonyl (e.g., methylsulfonyl), C₆₋₁₄ arylsulfonyl optionally having substituents, sulfamoyl, optionally halogenated C₁₋₆ alkylsulfinyl (e.g., methylsulfinyl), etc. Of these, preferred are, optionally halogenated C₁₋₆ alkyl-carbonyl, C₁₋₆ alkoxy-carbonyl, C₆₋₁₄ aryl-carbonyl optionally having substituents, C₆₋₁₄ arylsulfonyl optionally having substituents (e.g., benzenesulfonyl, 1-naphthalenesulfonyl, 2-naphthalenesulfonyl), etc.

For the “substituent” in said “C₆₋₁₄ aryl-carbonyl optionally having substituents”, “C₆₋₁₄ aryloxy-carbonyl optionally having substituents”, “C₇₋₁₉ aralkyloxy-carbonyl optionally having substituents”, “5- or 6-membered heterocyclic carbonyl optionally having substituents”, “C₆₋₁₄ aryl-carbamoyl optionally having substituents”, “5- or 6-membered heterocyclic carbamoyl optionally having substituents” and “C₆₋₁₄ arylsulfonyl optionally having substituents”, those similar to the “substituent” of the “5- to 7-membered heterocyclic group optionally having substituents” as mentioned above can be used. The number of the substituents is, for example, 1 to 3. When the number of the substituents is 2 or more, these substituents may be the same or different.

The “acylamino” which is a “substituent” of the ring A includes, for example, amino which is mono- or di-substituted by the above-mentioned “acyl” and preferably an acylamino represented by the formula: —NR⁹—COR¹⁰, —NR⁹—COOR^{10a}, —NR⁹—SO₂ R^{10a} or —NR⁹—CONR¹⁰R^{10b}, wherein R⁹ represents a hydrogen atom or a C₁₋₆ alkyl; R¹⁰ has the same meanings as the above R⁷; R^{10a} has the same meanings as the above R^{7a}; R^{10b} has the same meanings as the above R; etc.

The “C₁₋₆ alkyl” represented by R⁹ is exemplified by those mentioned as the “C₁₋₆ alkyl” represented by R⁸.

The preferable examples of the “acylamino” include formylamino, optionally halogenated C₁₋₆ alkyl-carboxamide (e.g. acetylamino), C₆₋₁₄ aryl-carboxamide optionally having substituents (e.g., phenylcarboxamide, naphthylcarboxamide), optionally halogenated C₁₋₆ alkoxy-carboxamide (e.g., methoxycarboxamide, ethoxycarboxamide, propoxycarboxamide, butoxycarboxamide), optionally halogenated C₁₋₆ alkylsulfonylamino (e.g., methylsulfonylamino, ethylsulfonylamino), etc.

Furthermore, for the “substituent” of the “C₆₋₁₄ aryl-carboxamide optionally having substituents”, those similar to the “substituent” of the “5- to 7-membered heterocyclic group optionally having substituents” as mentioned above can be used. The number of the substituents is, for example, 1 to 3. When the number of the substituents is 2 or more, these substituents may be the same or different.

The “acyloxy” which is a “substituent” of the ring A includes, for example, a hydroxy substituted by the above-mentioned “acyl”, and preferably an acyloxy represented by the formula: —O—COR¹¹, —O—COOR¹¹ or —O—CONHR¹¹, wherein R¹¹ has the same meanings as the above-mentioned R⁷; etc.

The preferable examples of the “acyloxy” include C₁₋₆ alkyl-carbonyloxy (e.g., acetoxy, propanoyloxy, isobutanoyloxy, pivaloyloxy), C_{6 14} aryl-carbonyloxy optionally having substituents (e.g., benzoyloxy, 1-naphthoyloxy, 2-naphthoyloxy), optionally halogenated C₁₋₆ alkoxy-carbonyloxy (e.g., methoxycarbonyloxy, trifluoromethoxycarbonyloxy, ethoxycarbonyloxy, propoxycarbonyloxy, butoxycarbonyloxy), mono- or di-C₁₋₆ alkyl-carbamoyloxy (e.g., methylcarbamoyloxy, ethylcarbamoyloxy, dimethylcarbamoyloxy, diethylcarbamoyloxy), C₆₋₁₄ aryl-carbamoyloxy optionally having substituents (e.g., phenylcarbamoyloxy, naphthylcarbamoyloxy), nicotinoyloxy, etc.

Furthermore, for the “substituent” of the “C₆₋₁₄ aryl-carbonyloxy optionally having substituents” and “C₆₋₁₄ aryl-carbamoyloxy optionally having substituents”, those similar to the “substituent” of the “5- to 7-membered heterocyclic group optionally having substituents” as mentioned above can be used. The number of the substituents is, for example, 1 to 3. When the number of the substituents is 2 or more, these substituents may be the same or different.

The “C₁₋₆ alkoxy-C₁₋₆ alkoxy” which is a “substituent” of the ring A includes, for example, methoxymethoxy, methoxyethoxy, ethoxymethoxy, ethoxyethoxy, propoxymethoxy, etc.

The “C₃₋₈ cycloalkyl-C₁₋₆ alkoxy” which is a “substituent” of the ring A includes, for example, cyclopropylmethoxy, cyclohexylmethoxy, etc.

The substituent in the ring A is preferably halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, mono- or di-C₁₋₆ alkyl-carbamoyl, optionally halogenated C₁₋₆ alkyl-carbonyl, optionally halogenated C₁₋₆ alkyl-sulfonyl, 5- to 7-membered non-aromatic heterocyclic group (preferably 1-pyrrolidinyl), C₁₋₆ alkoxy-C₁₋₆ alkoxy, 5- or 6-membered heterocyclic carbonyl (preferably pyrrolidin-1-ylcarbonyl), carboxy, C₁₋₆ alkoxy-carbonyl, 5- to 7-membered aromatic heterocyclic group (preferably thienyl, furyl, pyrazolyl) which may have substituents (preferably optionally halogenated C₁₋₆ alkyl, etc.), optionally halogenated C₁₋₆ alkylsulfinyl, C₃₋₈ cycloalkyl-C₁₋₆ alkoxy, etc.

The ring A is preferably a C₆₋₁₄ aromatic hydrocarbon or 5- or 6-membered aromatic heterocyclic ring (preferably benzene or thiazole; more preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated Cl ₆ alkyl-carboxamide, carbamoyl, mono- or di-C₁₋₆ alkyl-carbamoyl, optionally halogenated C₁₋₆ alkyl-carbonyl, optionally halogenated C₁₋₆ alkyl-sulfonyl, 5- to 7-membered non-aromatic heterocyclic group (preferably 1-pyrrolidinyl), C₁₋₆ alkoxy-Cl ₆ alkoxy, 5- or 6-membered heterocyclic carbonyl (preferably pyrrolidin-1-ylcarbonyl), carboxy, C₁₋₆ alkoxy-carbonyl, 5- to 7-membered aromatic heterocyclic group (preferably thienyl, furyl, pyrazolyl) which may have substituents (preferably optionally halogenated C₁₋₆ alkyl, etc.), optionally halogenated C₁₋₆ alkylsulfinyl, C₃₋₈ cycloalkyl-C₁₋₆ alkoxy, etc.

The “spacer having a main chain of 1 to 6 atoms” represented by B, Y and Ya means a spacer in which 1 to 6 atoms of a main chain are combined in a straight-chain form. The “number of atoms of a main chain” is counted so as the number of atoms of the main chain is minimum.

The “spacer having a main chain of 1 to 6 atoms” includes, for example, a divalent group comprising 1 to 5 groups selected from —O—, —S—, —CO—, —SO—, —SO₂—, —NR¹²—(R¹² is a hydrogen atom, C₁₋₆ alkyl optionally having substituents, C₁₋₆ alkyl-carbonyl optionally having substituents, or C₁₋₆ alkylsulfonyl optionally-having substituents) and a divalent C₁₋₆ non-cyclic hydrocarbon group optionally having substituents.

Said “C₁₋₆ alkyl” in the “C₁₋₆ alkyl optionally having substituents” includes, for example, methyl, ethyl, propyl, isopropyl, butyl, isobutyl, sec-butyl, tert-butyl, pentyl, hexyl, etc. Of these, preferred are methyl, ethyl, propyl, etc., and especially preferred is methyl.

The “C₁₋₆ alkyl-carbonyl” in the “C₁₋₆ alkyl-carbonyl optionally having substituents” includes, for example, acetyl, propanoyl, butanoyl, pentanoyl, hexanoyl, etc.

The “C₁₋₆ alkylsulfonyl” in the “C₁₋₆ alkylsulfonyl optionally having substituents” includes, for example, methylsulfonyl, ethylsulfonyl, propylsulfonyl, isopropylsulfonyl, butylsulfonyl, sec-butylsulfonyl, tert-butylsulfonyl, etc.

The “divalent C₁₋₆ non-cyclic hydrocarbon group” in the “divalent C₁₋₆ non-cyclic hydrocarbon group optionally having substituents” includes, for example,
(1) C₁₋₆ alkylene (e.g., —CH₂—, —(CH₂)₂—, —(CH₂)₃—, —(CH₂)₄—, —(CH₂)₅—, —(CH₂)₆—, —CH(CH₃)—, —C(CH₃)₂—, —CH(CH₃)CH₂—, —C(CH₃)₂CH₂—, —CH(CH₂CH₃)CH₂—, —(CH(CH₃))₂—, —(CH₂)₂C(CH₃)₂—, —CH₂C(CH₃)₂CH₂—, —CH(CH₂CH₃)(CH₂)₂—, —(CH₂)₃C(CH₃)₂—, —(CH₂)₃CH(CH₃)CH₂—);
(2) C₂₋₆ alkenylene (e.g., —CH═CH—, —CH₂—CH═CH—, —C(CH₃)₂—CH═CH—, —CH₂—CH═CH—CH₂—, —CH₂—CH₂—CH═CH—, —CH═CH—CH═CH—, —CH═CH—CH₂—CH₂—CH₂—);
(3) C₂₋₆ alkynylene (e.g., —C—C—, —CH₂—C═C—, —CH₂—C—C—CH₂—CH₂—).

The substituent in the “C₁₋₆ alkyl optionally having substituents”, “C₁₋₆ alkyl-carbonyl optionally having substituents”, “C₁₋₆ alkylsulfonyl optionally having substituents” and “divalent C₁₋₆ non-cyclic hydrocarbon group optionally having substituents” includes, for example, halogen atom, C₁₋₃ alkylenedioxy, nitro, cyano, optionally halogenated C₃₋₆ cycloalkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, C₁₋₆ alkoxy-carbonyl, mono- or di-C₁₋₆ alkyl-carbamoyl, optionally halogenated C₁₋₆ alkylsulfonyl, formylamino, optionally halogenated C₁₋₆ alkyl-carboxamide, C₁₋₆ alkoxy-carboxamide, C₁₋₆ alkylsulfonylamino, C₁₋₆ alkyl-carbonyloxy, C₁₋₆ alkoxy-carbonyloxy, mono- or di-C₁₋₆ alkyl-carbamoyloxy, etc. These substituents are exemplified by those mentioned as he “substituent” of the above “hydrocarbon group optionally having substituents”, etc. Of these, preferred are halogen atom, hydroxy, cyano, optionally halogenated C₁₋₆ alkoxy, etc. The number of the substituents is, for example, 1 to 3. When the number of the substituents is 2 or more, these substituents may be the same or different.

The preferable examples of the “spacer having a main chain of 1 to 6 atoms” include,
(1) C₁₋₆ alkylene which may have 1 to 3 substituents selected from halogen atom, hydroxy and cyano (e.g., —CH₂—, —CF₂—, —CCl₂—, —(CH₂)₂—, —(CH₂)₃—, —(CH₂)₄—, —(CH₂)₅—, —CH(CH₃)—, —CH(CN)—, —C(CH₃)₂—, —CH(CF₃)—, —CH(CH₃)CH₂—, —C(CH₃)₂CH₂—, —(CH(CH₃))₂—, —CH(CH₂CH₃)CH₂—, —(CF₂)₂—, —(CH₂)₂C(CH₃)₂—, —CH₂C(CH₃)₂CH₂—, —CH(CH₂CH₃)(CH₂)₂—, —(CH₂)₃C(CH₃)₂—, —(CH₂)₃CH(CH₃)CH₂—);
(2) C₂₋₆ alkenylene which may have 1 to 3 substituents selected from halogen atom, hydroxy and cyano (e.g., —CH═CH—, —CH₂—CH═CH—, —CH₂—CF═CH—, —C(CH₃)₂—CH═CH—, —CH₂—CH═CH—CH₂—, —CH₂—CH₂—CH═CH—, —CH═CH—CH═CH—, —CH═CH—CH₂—CH₂—CH₂—);
(3) C₂₋₆ alkynylene which may have 1 to 3 substituents selected from halogen atom, hydroxy and cyano (e.g., —C≡C—, —CH₂—C≡C—, —CH₂—C≡C—CH₂—CH₂—);
(4) spacer having a main chain of 1 to 6 atoms represented by the following formula:
   -alka-O-alkb-, -alka-S-alkb-, -alka-CO-alkb-,
   -alka-SO-alkb-, -alka-SO₂-alkb-, -alka-NR¹²-alkb-;
(5) spacer having a main chain of 2 to 6 atoms represented by the following formula:
   -alkc-CO-alkd-NR¹²-alke-, -alkc-NR¹²-alkd-CO-alke-,
   -alkc-SO₂-alkd-NR¹²-alke-, -alkc-NR¹²-alkd-SO₂-alke-,
   -alkc-CO-alkd-O-alke-, -alkc-O-alkd-CO-alke-,
   -alkc-CO-alkd-S-alke-, -alkc-S-alkd-CO-alke-;
(6) spacer having a main chain of 3 to 6 atoms represented by the following formula:
   -alkf-NR¹²CO-alkg-NR^{12a}-alkh-,
   -alkf-CONR¹²-alkg-NR^{12a}-alkh-,
   -alkf-CONR¹²-alkg-O-alkh-, -alkf-CONR¹²-alkg-S-alkh-,
   -alkf-NR¹²CO-alkg-O-alkh-, -alkf-NR¹²CO-alkg-S-alkh-,
   -alkf-SO₂NR¹²-alkg-O-alkh-, -alkf-SO₂NR¹²-alkg-S-alkh-,
   -alkf-NR¹²SO₂-alkg-O-alkh-, -alkf-NR¹²SO₂-alkg-S-alkh-,
   -alkf-CO-alkg-CONR¹²-alkh-, -alkf-CO-alkg-NR¹²CO-alkh-

(R¹² has the same meanings as above; R^{12a} has the same meanings as R¹²; alka, alkb, alkc, alkd, alke, alkf, alkg and alkh are the same or different and each represents a C₁₋₆ alkylene which may have 1 to 3 substituents selected from halogen atom, hydroxy and cyano or a bond), etc.

B is preferably C₁₋₆ alkylene, more preferably —CH₂—, —(CH₂)₂—, etc. Of these, preferred are —CH₂—, etc.

Y is preferably C₁₋₆ alkylene, -alka-CO-alkb-, -alkc-CO-alkd-O-alke- (each symbol is as defined above), etc; more preferably —CH₂—, —CO—, —CO—CH₂—CH₂—, —CO—CH₂—O—, etc. Of these, preferred are —CH₂—, —CO—, etc. Y is particularly preferably —CO—.

Ya is preferably a bond, etc.

The “hydrocarbon group optionally having substituents” represented by R¹ or R² is exemplified by those mentioned as the “substituent” of the above ring A.

The “heterocyclic group optionally having substituents” represented by R¹ or R² is exemplified by one mentioned as the above R⁷.

The “nitrogen-containing heterocyclic ring optionally having substituents” formed by R¹ and R² together with the adjacent nitrogen atom is exemplified by those mentioned as the “nitrogen-containing heterocyclic ring optionally having substituents” formed by the aforementioned R⁷ and R⁸ together with the adjacent nitrogen atom.

The “5- to 7-membered nitrogen-containing heterocyclic ring” formed by R¹ linked with ring A together with the adjacent nitrogen atom and B includes, for example, 5- to 7-membered nitrogen-containing heterocyclic ring containing at least one nitrogen atom in addition to carbon atoms and optionally further containing 1 to 3 heteroatoms selected from nitrogen, sulfur and oxygen atoms. Concrete examples are morpholine, thiomorpholine, piperidine, piperazine, pyrrolidine, azepane; and unsaturated cyclic amines thereof (e.g., 1,2,5,6-tetrahydropyridine), etc. Of these, preferred are morpholine, piperidine, piperazine, pyrrolidine, etc.

R¹ and R² are preferably a hydrogen atom, C₁₋₆ alkyl or C₃₋₈ cycloalkyl; more preferably C₁₋₆ alkyl. Of these, preferred is methyl.

The “hydrocarbon group optionally having substituents” represented by R³ is exemplified by those mentioned as the “substituent” of the above ring A.

The “heterocyclic group optionally having substituents” represented by R³ is exemplified by one mentioned as the above R⁷.

R³ is preferably a hydrogen atom or a C₁₋₆ alkyl optionally having substituents, more preferably a hydrogen atom. The “C₁₋₆ alkyl optionally having substituents” here is preferably a C₁₋₆ alkyl optionally having 1 to 3 substituents selected from halogen atom, hydroxy and cyano, more preferably C₁₋₆ alkyl (preferably methyl).

The “hydrocarbon group optionally having substituents” represented by R⁴ or R⁵ is exemplified by those mentioned as the “substituent” of the above ring A. The “hydrocarbon group optionally having substituents” is preferably a C₁₋₆ alkyl optionally having substituents, more preferably a C₁₋₆ alkyl optionally having 1 to 3 substituents selected from halogen atom, hydroxy and cyano.

The “ring” in the “ring optionally having substituents” formed by R⁴ and R⁵ together with the adjacent carbon atom includes, for example, C₃₋₆ cycloalkane, 5- to 10-membered non-aromatic heterocyclic ring, etc.

The C₃₋₆ cycloalkane here includes, for example, cyclopropane, cyclobutane, cyclopentane, cyclohexane, etc.

The 5- to 10-membered non-aromatic heterocyclic ring includes, for example, 5- to 10-membered non-aromatic heterocyclic ring containing, in addition to carbon atoms, 1 to 4 heteroatoms selected from nitrogen, sulfur and oxygen atoms, etc. The preferable examples of the non-aromatic heterocyclic ring include pyrrolidine, imidazolidine, imidazoline, pyrazolidine, piperidine, piperazine, morpholine, thiomorpholine, etc.

The “substituent” of the “ring optionally having substituents” is exemplified by those mentioned as the “substituent” of the above “5- to 7-membered heterocyclic group optionally having substituents”. The number of the substituents is, for example, 1 to 3. When the number of the substituents is 2 or more, these substituents may be the same or different.

In compound (I), it is preferable that one of R⁴ and R⁵ is a hydrogen atom and the other is a C₁₋₆ alkyl optionally having substituents. The “C₁₋₆ alkyl optionally having substituents” is preferably a C₁₋₆ alkyl optionally having 1 to 3 substituents selected from halogen atom, hydroxy and cyano, more preferably C₁₋₆ alkyl. The most preferred is methyl.

For the “substituent” of said “indolyl group optionally having substituents” represented by R⁶, those similar to the “substituent” of the “5- to 7-membered heterocyclic group optionally having substituents” as mentioned above can be used. The substituent is preferably halogen atom, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, C₁₋₆ alkoxy-carbonyl, mono- or di-C₁₋₆ alkyl-carbamoyl, optionally halogenated C₁₋₆ alkylsulfonyl, sulfamoyl, formylamino, optionally halogenated C₁₋₆ alkyl-carboxamide, C₁₋₆ alkoxy-carboxamide, C₁₋₆ alkylsulfonylamino, etc.

The number of the substituents is, for example, 1 to 3. When the number of the substituents is 2 or more, these substituents may be the same or different.

In addition, the indolyl group is preferably 2-indolyl or 3-indolyl, more preferably 3-indolyl.

R⁶ is preferably 3-indolyl.

The “halogen atom” represented by Z and Za includes fluorine, chlorine, bromine, iodine, etc.

The “cyclic group” of the “cyclic group optionally having substituents” represented by Z and Za includes, for example, non-aromatic cyclic hydrocarbon group, aromatic hydrocarbon group, non-aromatic heterocyclic group, aromatic heterocyclic group, etc.

The aromatic hydrocarbon group is exemplified by C₆₋₁₄ aryl, etc. mentioned as the “hydrocarbon group” of the above “hydrocarbon group optionally having substituents”.

The non-aromatic cyclic hydrocarbon group includes, for example, C₃₋₈ cycloalkyl which may be condensed with benzene ring, C₃₋₈ cycloalkenyl which may be condensed with benzene ring, etc. The “C₃₋₈ cycloalkyl which may be condensed with benzene ring” and the “C₃₋₈ cycloalkenyl which may be condensed with benzene ring” are exemplified by those mentioned as the “hydrocarbon group” of the above “hydrocarbon group optionally having substituents”.

The non-aromatic heterocyclic group and the aromatic heterocyclic group are exemplified by those mentioned as the “heterocyclic group” of the above “heterocyclic group optionally having substituents” represented by R⁷.

The “cyclic group” is preferably non-aromatic heterocyclic group, more preferably 4- to 10-membered mono-cyclic non-aromatic heterocyclic group or 4- to 10-membered fused bi-cyclic non-aromatic heterocyclic group. Of these, preferred are piperidinyl (including oxopiperidinyl and dioxopiperidinyl), piperazinyl (including oxopiperazinyl and dioxopiperazinyl), tetrahydropyridinyl, indolinyl, isoindolinyl, tetrahydroisoquinolinyl, tetrahydroquinolinyl, tetrahydrothieno[2,3-c]pyridinyl, tetrahydrobenzazepinyl, pyrrolidinyl, etc. Especially preferred are piperidinyl (including oxopiperidinyl and dioxopiperidinyl; preferably 1-piperidinyl), piperazinyl (including oxopiperazinyl and dioxopiperazinyl; preferably 1-piperazinyl), etc.

The “substituent” of the “cyclic group optionally having substituents” represented by Z and Za includes, for example, halogen atom, C₁₋₃ alkylenedioxy, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, C₁₋₆ alkoxy-carbonyl, mono- or di-C₁₋₆ alkyl-carbamoyl, optionally halogenated C₁₋₆ alkylsulfonyl, sulfamoyl, formylamino, optionally halogenated C₁₋₆ alkyl-carboxamide, C₁₋₆ alkoxy-carboxamide, C₁₋₆ alkylsulfonylamino, C₁₋₆ alkyl-carbonyloxy, C₁₋₆ alkoxy-carbonyloxy, mono- or di-C₁₋₆ alkyl-carbamoyloxy, etc. These substituents are exemplified by those mentioned as the substituent of the above “5- to 7-membered heterocyclic group optionally having substituents”.

The number of the substituents is, for example, 1 to 3. When the number of the substituents is 2 or more, these substituents may be the same or different.

The “substituent” of the above “cyclic group optionally having substituents” also includes a group of the formula: -Yd-Ar (Yd represents a bond or a spacer having a main chain of 1 to 6 atoms and Ar represents an aromatic group optionally having substituents); a group of the formula: -Yd-Ara (Yd represents a bond or a spacer having a main chain of 1 to 6 atoms and Ara represents a monocyclic group optionally having substituents), etc.

The “spacer having a main chain of 1 to 6 atoms” represented by Yd is exemplified by those mentioned as the above “B”.

Of these, preferred are C₁₋₆ alkylene, -alka-O-alkb-, -alka-S-alkb-, -alka-CO-alkb-, -alka-SO-alkb-, -alka-SO₂-alkb- (each symbol is as defined above), etc.

Yd is preferably a bond, C₁₋₆ alkylene, -alka-O-alkb-, -alka-S-alkb-, -alka-CO-alkb-, -alka-SO-alkb-, -alka-SO₂-alkb-, -alkc-CO-alkd-NR¹ 2-alke- (each symbol is as defined above); more preferably a bond, C₁₋₆ alkylene, —O—, —S—, —CO—, —SO₂—, —CONH—, etc.

The “aromatic group” of the “aromatic group optionally having substituents” represented by Ar includes, for example, aromatic hydrocarbon group, aromatic heterocyclic group, etc.

The aromatic hydrocarbon group is exemplified by C₆₋₁₄ aryl, etc. mentioned as the “hydrocarbon group” of the above “hydrocarbon group optionally having substituents”.

The aromatic heterocyclic group is exemplified by those mentioned as the “heterocyclic group” of the “heterocyclic group optionally having substituents” represented by the above R⁷.

The “aromatic group” is preferably C₆₋₁₄ aryl (preferably phenyl), 5- or 6-membered aromatic heterocyclic group (e.g., tetrazolyl, thiazolyl, oxazolyl, furyl, thienyl, pyridyl), etc.

Of these, C₆₋₁₄ aryl (preferably phenyl) is preferred. In addition, the “aromatic group” is preferably a monocyclic aromatic group.

The “monocyclic group” of the “monocyclic group optionally having substituents” represented by Ara includes, for example, phenyl, C₃₋₈ cycloalkyl, C₃₋₈ cycloalkenyl, monocyclic aromatic heterocyclic group, monocyclic non-aromatic heterocyclic group, etc.

As used herein, the C₃₋₈ cycloalkyl includes, for example, cyclopropyl, cyclobutyl, cyclopentyl, cyclohexyl, cycloheptyl, cyclooctyl, dihydroindenyl, etc.

The C₃₋₈ cycloalkenyl includes, for example, cyclopropenyl, cyclobutenyl, cyclopentenyl, cyclohexenyl, cycloheptenyl, cyclooctenyl, etc.

The monocyclic aromatic heterocyclic group includes monocyclic ones from the aromatic heterocyclic groups exemplified for the aforementioned “heterocyclic group” represented by the above R⁷.

The monocyclic non-aromatic heterocyclic group includes monocyclic ones from the non-aromatic heterocyclic groups exemplified for the aforementioned “heterocyclic group” represented by the above R⁷.

The “monocyclic group” is preferably phenyl, C₃₋₈ cycloalkyl (preferably cyclopropyl, cyclobutyl, cyclopentyl, cyclohexyl), 5- or 6-membered monocyclic aromatic heterocyclic group (preferably tetrazolyl, thiazolyl, oxazolyl, furyl, thienyl, pyridyl), etc.

The “substituent” of the “aromatic group optionally having substituents” represented by Ar and the “monocyclic group optionally having substituents” represented by Ara includes, for example, halogen atom, C₁₋₃ alkylenedioxy, nitro, cyano, optionally halogenated C₁₋₆ alkyl, optionally halogenated C₃₋₆ cycloalkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, C₁₋₆ alkoxy-carbonyl, mono- or di-C₁₋₆ alkyl-carbamoyl, optionally halogenated C₁₋₆ alkylsulfonyl, sulfamoyl, formylamino, optionally halogenated C₁₋₆ alkyl-carboxamide, C₁₋₆ alkoxy-carboxamide, C₁₋₆ alkylsulfonylamino, C₁₋₆ alkyl-carbonyloxy, C₁₋₆ alkoxy-carbonyloxy, mono- or di-C₁₋₆ alkyl-carbamoyloxy, hydroxy-C₁₋₆ alkyl, etc. These substituents are exemplified by those mentioned as the “substituent” of the above “5- to 7-membered heterocyclic group optionally having substituents”.

The number of the substituents is, for example, 1 to 3. When the number of the substituents is 2 or more, these substituents may be the same or different.

Ar is preferably a C₆₋₁₄ aryl (preferably phenyl) or 5- or 6-membered aromatic heterocyclic group (e.g., tetrazolyl, thiazolyl, oxazolyl, furyl, thienyl, pyridyl) [more preferably C₆₋₁₄ aryl and most preferably phenyl], each of which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, hydroxy-C₁₋₆ alkyl, etc.

Ara is preferably a phenyl, a C₃₋₈ cycloalkyl (preferably cyclopropyl, cyclobutyl, cyclopentyl, cyclohexyl) or a 5- or 6-membered monocyclic aromatic heterocyclic group (preferably tetrazolyl, thiazolyl, oxazolyl, furyl, thienyl, pyridyl) [more preferably phenyl], each of which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, hydroxy-C₁₋₆ alkyl, etc.

When the “cyclic group” represented by Z and Za is 1-piperidinyl (including oxopiperidin-1-yl and dioxopiperidin-1-yl) or 1-piperazinyl (including oxopiperazin-1-yl and dioxopiperazin-1-yl), these preferably have a “substituent” at 4-position.

The “substituent” of the “cyclic group optionally having substituents” represented by Z and Za is preferably hydroxy, a group of the formula: -Yd-Ar (Yd and Ar have the same meanings as above) and a group of the formula: -Yd-Ara (Yd and Ara have the same meanings as above).

Z is preferably a cyclic group optionally having substituents; more preferably piperidinyl (including oxopiperidinyl and dioxopiperidinyl) or piperazinyl (including oxopiperazinyl and dioxopiperazinyl), each of which has 1 or 2 substituents selected from hydroxy, a group of the formula: -Yd-Ar (Yd and Ar have the same meanings as above) and a group of the formula: -Yd-Ara (Yd and Ara have the same meanings as above); particularly preferably 1-piperidinyl (including oxopiperidin-1-yl and dioxopiperidin-1-yl) or 1-piperazinyl (including oxopiperazin-1-yl and dioxopiperazin-1-yl), each of which has a group of the formula: -Yd-Ar (Yd and Ar have the same meanings as above) or a group of the formula: -Yd-Ara (Yd and Ara have the same meanings as above) at 4-position.

Za is preferably a hydrogen atom.

The preferable examples of compound (I) include the following compounds.

[Compound A]

A compound wherein

ring A is a C₆₋₁₄ aromatic hydrocarbon or a 5- or 6-membered aromatic heterocyclic ring (preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, optionally halogenated C₁₋₆ alkyl-carbonyl, optionally halogenated C₁₋₆ alkyl-sulfonyl, etc.;

B is a C₁₋₆ alkylene (preferably —CH₂—);

Y is a C_{1 6} alkylene (preferably —CH₂—) or -alka-CO-alkb- (each symbol is as defined above; preferably —CO—);

Ya is a bond;

R¹ and R² are the same or different and each is a C₁₋₆ alkyl (preferably methyl);

R³ is a hydrogen atom or a C₁₋₆ alkyl;

one of R⁴ and R⁵ is a hydrogen atom, and the other is a C₁₋₆ alkyl (preferably methyl);

R⁶ is 3-indolyl;

Z is 1-piperidinyl or 1-piperazinyl, each of which has a group of the formula: -Yd-Ar [Yd is preferably a bond, C₁₋₆ alkylene, —O—, —S—, —CO—, —SO₂—; Ar is preferably a C₆₋₁₄ aryl (preferably phenyl) which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, etc.] at 4-position; and

Za is a hydrogen atom.

[Compound B]

A compound wherein

ring A is a C₆₋₁₄ aromatic hydrocarbon or a 5- or 6-membered aromatic heterocyclic ring (preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, optionally halogenated C₁₋₆ alkyl-carbonyl, optionally halogenated C₁₋₆ alkyl-sulfonyl, etc.;

B is a C₁₋₆ alkylene (preferably —CH₂—);

Y is a C₁₋₆ alkylene (preferably —CH₂—) or -alka-CO-alkb- (each symbol is as defined above; preferably —CO—)

Ya is a bond;

R¹ and R² form, together with the adjacent nitrogen atom, a 3- to 8-membered nitrogen-containing heterocyclic ring containing at least one nitrogen atom in addition to carbon atoms and optionally further containing 1 to 3 heteroatoms selected from nitrogen, sulfur and oxygen atoms (preferably morpholine, piperidine, piperazine and pyrrolidine);

R³ is a hydrogen atom or a C₁₋₆ alkyl;

one of R⁴ and R⁵ is a hydrogen atom, and the other is a C₁₋₆ alkyl (preferably methyl);

R⁶ is 3-indolyl;

Z is 1-piperidinyl or 1-piperazinyl, each of which has a group of the formula: -Yd-Ar [Yd is preferably a bond, C₁₋₆ alkylene, —O—, —S—, —CO— or —SO₂—; Ar is preferably a C₆₋₁₄ aryl (preferably phenyl) which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, etc.] at 4-position; and

Za is a hydrogen atom.

[Compound C]

A compound wherein

ring A is a C₆₋₁₄ aromatic hydrocarbon or a 5- or 6-membered aromatic heterocyclic ring (preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, optionally halogenated C₁₋₆ alkyl-carbonyl, optionally halogenated C₁₋₆ alkyl-sulfonyl, etc.;

B is a C₁ 6 alkylene (preferably —CH₂—);

Y is a C₁₋₆ alkylene (preferably —CH₂—) or -alka-CO-alkb- (each symbol is as defined above; preferably —CO—);

Ya is a bond;

R¹ and R² are the same or different and each is a C₁₋₆ alkyl (preferably methyl);

R³ is a hydrogen atom or a C₁₋₆ alkyl;

one of R⁴ and R⁵ is a hydrogen atom, and the other is a C₁₋₆ alkyl (preferably methyl);

R⁶ is 3-indolyl;

Z is a 4- to 10-membered fused bi-cyclic non-aromatic heterocyclic group (preferably indolinyl, isoindolinyl, tetrahydroisoquinolinyl, tetrahydroquinolinyl, tetrahydrothieno[2,3-c)pyridinyl, tetrahydrobenzazepinyl) which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, etc.; and

Za is a hydrogen atom.

[Compound D]

A compound wherein

ring A is a C₆₋₁₄ aromatic hydrocarbon or a 5- or 6-membered aromatic heterocyclic ring (preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C_{1 6} alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, optionally halogenated C₁₋₆ alkyl-carbonyl, optionally halogenated C₁₋₆ alkyl-sulfonyl, 5- to 7-membered non-aromatic heterocyclic group (preferably 1-pyrrolidinyl), C₁₋₆ alkoxy-C₁₋₆ alkoxy, etc.;

B is a C₃₋₆ alkylene (preferably —CH₂—, —(CH₂)₂—; more preferably —CH₂—);

Y is a C₁₋₆ alkylene (preferably —CH₂—) or -alka-CO-alkb- (each symbol is as defined above; preferably —CO—);

Ya is a bond;

R¹ and R² are the same or different and each represents a hydrogen atom, a C₁₋₆ alkyl or a C₃₋₈ cycloalkyl (preferably C₁₋₆ alkyl);

R³ is a hydrogen atom or a C₁₋₆ alkyl;

one of R⁴ and R⁵ is a hydrogen atom, and the other is a C₁₋₆ alkyl (preferably methyl);

R⁶ is 3-indolyl;

Z is 1-piperidinyl or 1-piperazinyl, each of which has 1 or 2 substituents selected from hydroxy, optionally halogenated C₃₋₆ cycloalkyl (preferably cyclohexyl) and a group of the formula: -Yd-Ar [Yd is preferably a bond, C₁₋₆ alkylene, —O—, —S—, —CO—, —SO₂—; Ar is preferably a C₆₋₁₄ aryl (preferably phenyl) or a 5- or 6-membered aromatic heterocyclic group (e.g., tetrazolyl, thiazolyl, oxazolyl) [more preferably a C₆₋₁₄ aryl and most preferably phenyl], each of which may have 1 to 3 substituents selected from a halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, etc.] at 4-position; and

Za is a hydrogen atom.

[Compound E]

A compound wherein

ring A is a C₆₋₁₄ aromatic hydrocarbon or a 5- or 6-membered aromatic heterocyclic ring (preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, optionally halogenated C₁₋₆ alkyl-carbonyl, optionally halogenated C₁₋₆ alkyl-sulfonyl, 5- to 7-membered non-aromatic heterocyclic group (preferably 1-pyrrolidinyl), C₁₋₆ alkoxy-C₁₋₆ alkoxy, etc.;

B is a C₁₋₆ alkylene (preferably —CH₂—, —(CH₂)₂—; more preferably —CH₂—),

Y is a C₁₋₆ alkylene (preferably —CH₂—) or -alka-CO-alkb- (each symbol is as defined above; preferably —CO—);

Ya is a bond;

R¹ is linked with ring A together with the adjacent nitrogen atom and B to form a 5- to 7-membered nitrogen-containing heterocyclic ring (preferably piperidine, pyrrolidine, azepane);

R² is a hydrogen atom, a C₁₋₆ alkyl or a C₃₋₈ cycloalkyl (preferably a C₁₋₆ alkyl);

R³ is a hydrogen atom or a C₁₋₆ alkyl;

one of R⁴ and R⁵ is a hydrogen atom, and the other is a C₁₋₆ alkyl (preferably methyl);

R⁶ is 3-indolyl;

Z is 1-piperidinyl or 1-piperazinyl, each of which has 1 or 2 substituents selected from hydroxy, optionally halogenated C₃₋₆ cycloalkyl (preferably cyclohexyl) and a group of the formula: -Yd-Ar [Yd is preferably a bond, C₁₋₆ alkylene, —O—, —S—, —CO— or —SO₂—; Ar is preferably a C₆₋₁₄ aryl (preferably phenyl) or 5- or 6-membered aromatic heterocyclic group (e.g., tetrazolyl, thiazolyl, oxazolyl) [more preferably a C₆₋₁₄ aryl and most preferably phenyl], each of which may have 1 to 3 substituents selected from a halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, etc.] at 4-position; and

Za is a hydrogen atom.

[Compound F]

A compound wherein

ring A is a C₆₋₁₄ aromatic hydrocarbon or a 5- or 6-membered aromatic heterocyclic ring (preferably benzene or thiazole and more preferably benzene), each of which may have 1 to 3 substituents selected from halogen atom, nitro, cyano, hydroxy, optionally halogenated C₁₋₆ alkyl, C₆₋₁₄ aryl which may have substituents (preferably halogen atom, hydroxy, optionally halogenated C₁₋₆ alkyl, optionally halogenated C₁₋₆ alkoxy, optionally halogenated C₁₋₆ alkyl-carbonyl, etc.), optionally halogenated C₁₋₆ alkoxy, optionally halogenated C₁₋₆ alkylthio, amino, mono- or di-C₁₋₆ alkylamino, optionally halogenated C₁₋₆ alkyl-carboxamide, carbamoyl, mono- or di-C₁₋₆ alkyl-carbamoyl, optionally halogenated C₁₋₆ alkyl-carbonyl, optionally halogenated C₁₋₆ alkyl-sulfonyl, 5- to 7-membered non-aromatic heterocyclic group (preferably 1-pyrrolidinyl), C₁₋₆ alkoxy-C₁₋₆ alkoxy, 5- or 6-membered heterocyclic carbonyl (preferably pyrrolidin-1-ylcarbonyl), carboxy, C₁₋₆ alkoxy-carbonyl, 5- to 7-membered aromatic heterocyclic group (preferably thienyl, furyl, pyrazolyl) which may have substituents (preferably optionally halogenated C₁₋₆ alkyl, etc.), optionally halogenated C₁₋₆ alkylsulfinyl, C₃₋₈ cycloalkyl-C₁₋₆ alkoxy, etc.;

B is C₁₋₆ alkylene (preferably —CH₂—, —(CH₂)₂—; more preferably —CH₂—);

Y is C₁₋₆ alkylene, -alka-CO-alkb- or -alkc-CO-alkd-O-alke- (each symbol is as defined above; preferably —CH₂—, —CO—, —CO—CH₂—CH₂—, —CO—CH₂—O—, etc.; more preferably —CO—);

Ya is a bond;

R¹ and R² are the same or different and each is a hydrogen atom, a C₁₋₆ alkyl or a C₃₋₈ cycloalkyl (preferably a C₁₋₆ alkyl);

R³ is a hydrogen atom or a C₁₋₆ alkyl;

one of R⁴ and R⁵ is a hydrogen atom, and the other is a C₁₋₆ alkyl (preferably methyl);

R⁶ is 3-indolyl;

Z is 1-piperidinyl (including oxopiperidin-1-yl and dioxopiperidin-1-yl) or 1-piperazinyl (including oxopiperazin-1-yl and dioxopiperazin-1-yl), each of which has 1 or 2 substituents selected from (1) hydroxy, (2) a group of the formula: -Yd-Ar [Yd is preferably a bond, C₁₋₆ alkylene, —O—, —S—, —CO—, —SO₂— or —CONH—; Ar is preferably a C₆₋₁₄ aryl (preferably phenyl) or a 5- or 6-membered aromatic heterocyclic group (e.g., tetrazolyl, thiazolyl, oxazolyl, furyl, thienyl, pyridyl) [more preferably C₆₋₁₄ aryl and most preferably phenyl], each of which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, hydroxy-C₁₋₆ alkyl, etc.,] and (3) a group of the formula: -Yd-Ar [Yd is preferably a bond, C₁₋₆ alkylene, —O—, —S—, —CO—, —SO₂— or —CONH—; Ara is preferably a phenyl, a C₃₋₈ cycloalkyl (preferably cyclopropyl, cyclobutyl, cyclopentyl, cyclohexyl) or a 5- or 6-membered monocyclic aromatic heterocyclic group (preferably tetrazolyl, thiazolyl, oxazolyl, furyl, thienyl, pyridyl) [more preferably phenyl], each of which may have 1 to 3 substituents selected from halogen atom, nitro, cyano, optionally halogenated C₁₋₆ alkyl, optionally halogenated C₁₋₆ alkoxy, optionally halogenated C₁₋₆ alkylthio, hydroxy, amino, mono- or di-C₁₋₆ alkylamino, formyl, carboxy, carbamoyl, thiocarbamoyl, optionally halogenated C₁₋₆ alkyl-carbonyl, optionally halogenated C₁₋₆ alkylsulfonyl, sulfamoyl, hydroxy-C₁₋₆ alkyl, etc.,] at 4-position; and

Za is a hydrogen atom.

[Compound G]

N-((1R,2S)-1-(((5-((dimethylamino)methyl)-2-((methylamino)carbonyl)phenyl)amino)carbonyl)-2-(1H-indol-3-yl)propyl)-4-(2-methylphenyl)-1-piperidinecarboxamide (Example 193);

N-((1R,2S)-1-(((2-((dimethylamino)carbonyl)-5-((dimethylamino)methyl)phenyl)amino)carbonyl)-2-(1H-indol-3-yl)propyl)-4-(4-fluorophenyl)-1-piperidinecarboxamide (Example 203);

N-((1R,2S)-1-(((5-((dimethylamino)methyl)-2-methoxyphenyl)amino)carbonyl)-2-(1H-indol-3-yl)propyl)-4-(4-fluoro-2-methylphenyl)-3-oxo-1-piperazinecarboxamide (Example 179);

N-((1R,2S)-1-((.(5-((dimethylamino)methyl)-2-methoxyphenyl)amino)carbonyl)-2-(1H-indol-3-yl)propyl)-4-(2-methylphenyl)-1-piperazinecarboxamide (Example 75);

N-((1R,2S)-1-(((5-((dimethylamino)methyl)-2-ethoxyphenyl)amino)carbonyl)-2-(1H-indol-3-yl)propyl)-4-(4-fluorophenyl)-1-piperazinecarboxamide (Example 67); or

N-((1R,2S)-1-(((5-((dimethylamino)methyl)-2-ethoxyphenyl)amino)carbonyl)-2-(1H-indol-3-yl)propyl)-4-phenyl-1-piperidinecarboxamide (Example 54).

When the compound (I) is in the form of a salt, concrete examples thereof include salts with inorganic bases, ammonium salts, salts with organic bases, salts with inorganic acids, salts with organic acids and salts with basic or acidic amino acids.

Preferable examples of the salts with inorganic bases include alkali metal salts such as sodium salt, potassium salt, etc; alkaline earth metal salts such as calcium salts, magnesium salts, barium salts, etc; aluminum salts, etc.

Preferable examples of the salts with organic bases include salts with trimethylamine, triethylamine, pyridine, picoline, ethanolamine, diethanolamine, triethanolamine, dicyclohexylamine, N,N′-dibenzylethylenediamine, etc.

Preferable examples of the salts with inorganic acids include salts with hydrochloric acid, hydrobromic acid, nitric acid, sulfuric acid, phosphoric acid, etc.

Preferable examples of the salts with organic acids include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, etc.

Preferable examples of the salts with basic amino acids include salts with arginine, lysine, ornithine, etc.

Preferable examples of the salts with acidic amino acids include salts with aspartic acid, glutamic acid, etc.

Of these, pharmaceutically acceptable salts are preferable.

Preferable examples when compound (I) has an acidic functional group include inorganic salts such as alkali metal salts (e.g., sodium salt, potassium salt, etc.) and alkaline earth metal salts (e.g., calcium salt, magnesium salt, barium salt, etc.), ammonium salts, etc; and when compound (I) has a basic functional group, inorganic salts such as hydrochloride, sulfate, phosphate and hydrobromide; or organic salts such as acetate, maleate, fumarate, succinate, methanesulfonate, p-toluenesulfonate, citrate and tartrate.

The prodrug of the compound (I) means a compound capable of being converted to the compound (I) in vivo by the action of an enzyme or gastric juice and the like under physiological conditions, namely a compound capable of being converted to the compound (I) upon enzymatic oxidation, reduction or hydrolysis and the like, or a compound capable of being converted to the compound (I) upon hydrolysis and the like by gastric juice and the like. As the prodrug of the compound (I), compounds derived by acylation, alkylation or phosphorylation of the amino group of the compound (I) (e.g. compounds derived by eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation or tert-butylation of the amino group of the compound of the compound (I), and the like); compounds derived by acylation, alkylation, phosphorylation or boration of the hydroxy group of the compound (I) (e.g. compounds derived by acetylation, palmitoylation, propanoylation, pivaloylation, succinylation, fumarylation, alanylation or dimethylaminomethylcarbonylation of the hydroxy group of the compound (I), and the like); and compounds derived by esterification or amidation of the carboxyl group of the compound (I) (e.g. compounds derived by ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-l,3-dioxolen-4-yl)methyl esterification, cyclohexyloxycarbonylethyl esterification, or methylamidation of the carboxyl group of the compound (I), and the like), and the like can be mentioned. These compounds can be produced from the compound (I) by methods known per se.

The prodrug of the compound (I) may be one capable of being converted to the compound (I) under physiological conditions, as described in “Iyakuhin no Kaihatsu (Development of Drugs)”, vol. 7, Molecular Designing, published by Hirokawa Shoten, 1990, pages 163-198.

The process for producing the compound (I) is mentioned below.

The compound (I) can be produced by a means known per se, for example, by the methods exemplified in the following scheme 1 or 2, or a method similar thereto, etc.

Compounds described in the following schemes may be in the form of salts. Such salts are exemplified by those similar to the salts of the compound (I).

“Room temperature” is normally meant to indicate a temperature falling between 0° C. and 30° C. in the present specification.

The following reaction such as alkylation, hydrolysis, amination, esterification, amidation, etherification, oxidation, reduction, urea formation, etc. may be conducted according to methods known per se, for example, those described in Organic Functional Group Preparations, 2nd Ed., Academic Press Inc., 1989 and in Comprehensive Organic Transformations, VCH Publishers Inc., 1989. or a similar method thereto. 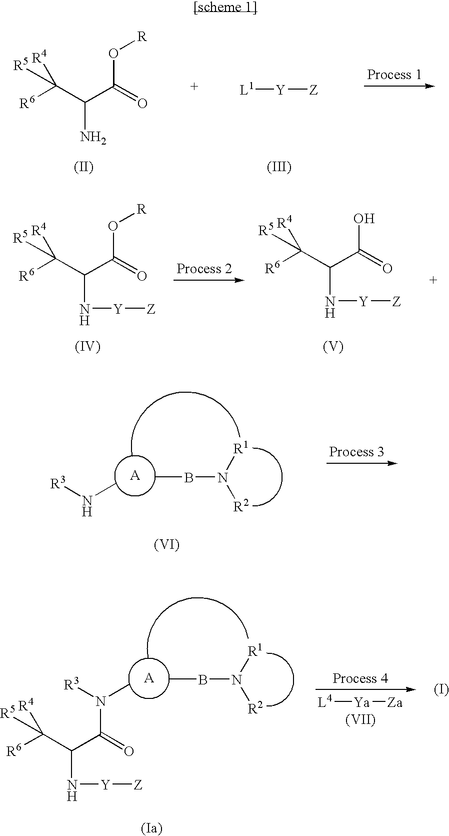
wherein R represents a protective group of carboxyl group, L¹ and L⁴ are the same or different and each represents a leaving group, and the other symbols are as defined above.

The protective group of carboxyl group represented by R includes, for example, C₁₋₆ alkyl (e.g., methyl, ethyl, propyl, isopropyl, butyl, tert-butyl, etc.), C₇₋₁₁ aralkyl (e.g. , benzyl, etc.), phenyl, trityl, silyl (e.g., trimethylsilyl, triethylsilyl, dimethylphenylsilyl, tert-butyldimethylsilyl, tert-butyldiethylsilyl, etc.), C₂₋₆ alkenyl (e.g., 1-allyl, etc.), etc. These groups may be substituted by 1 to 3 halogen atoms (e.g., fluorine, chlorine, bromine, iodine, etc.), C₁₋₆ alkoxy (e.g., methoxy, ethoxy, propoxy, etc.) or nitro, etc. The protective group of carboxyl group is preferably methyl, ethyl, etc.

The “leaving group” represented by L¹ or L⁴ includes, for example, (1) halogen atom (e.g., chlorine, bromine, iodine, etc.), (2) optionally halogenated C₁₋₆ alkylsulfonyloxy (e.g., methanesulfonyloxy, ethanesulfonyloxy, trifluoromethanesulfonyloxy, etc.), (3) C₆₋₁₀ arylsulfonyloxy optionally having substituents, (4) hydroxy, (5) succinimidoxy, etc.

The “substituent” of the “C₆₋₁₀ arylsulfonyloxy optionally-having substituents” includes, for example, halogen atom, optionally halogenated C₁₋₆ alkyl, optionally halogenated C₁₋₆ alkoxy, etc. The number of the substituents is, for example, 1 to 3.

Concrete examples of the “C₆₋₁₀ arylsulfonyloxy optionally having substituents” include benzenesulfonyloxy, p-toluenesulfonyloxy, 1-naphthalenesulfonyloxy, 2-naphthalenesulfonyloxy, etc.

Each process of scheme 1 is described in detail in the following.

Process 1: Induction of the group represented by the formula: —Y-Z (each symbol is as defined above)

By the present process, compound (IV) can be produced by reacting compound (II) with compound (III) or a reactive derivative thereof.

When a functional group adjacent to the leaving group L¹ is CO, SO or SO₂ for Y in compound (III), the present process is conducted by amidation.

Said “amidation” includes, for example, the below mentioned method such as i) the method using a dehydrating/condensing agent or ii) the method using a reactive derivative of carboxy.

i) The Method Using a Dehydrating/Condensing Agent

Compound (II), about 1 to about 5 equivalents of Compound (III), and about 1 to about 2 equivalents of a dehydrating/condensing agent are reacted in an inert solvent.

Said “dehydrating/condensing agent” includes; for example, dicyclohexylcarbodiimide (DCC), 1-ethyl-3-(3-dimethylaminopropyl)carbodiimide hydrochloride (WSC), etc. Of these, WSC is preferred.

The “inert solvent” includes, for example, nitriles, amides, halogenated hydrocarbons, ethers, etc. These may be used on mixing two or more kinds at a suitable proportion. Of these, preferred is acetonitrile, DMF, dichloromethane, THF, etc.

The reaction temperature is generally about −20° C. to about 50° C., preferably at room temperature.

The reaction time is generally about 10 hours to about 24 hours.

In the present reaction, about 1 to about 1.5 equivalents of 1-hydroxybenzotriazole (HOBt) or 1-hydroxy-7-azabenzotriazole (HOAt) is used as necessary.

In the present reaction, about 1 to about 5 equivalents of a base is also used as necessary.

Said “base” includes, for example;

1) strong bases such as alkali metal or alkaline earth metal hydrides (e.g., lithium hydride, sodium hydride, potassium hydride, calcium hydride, etc.), alkali metal or alkaline earth metal amides (e.g., lithium amide, sodium amide, lithium diisopropylamide, lithium dicyclohexylamide, lithium hexamethyldisilazide, sodium hexamethyldisilazide, potassium hexamethyldisilazide, etc.), alkali metal or alkaline earth metal lower-alkoxides (e.g., sodium methoxide, sodium ethoxide, potassium tert-butoxide, etc.), etc.;

2) inorganic bases such as alkali metal or alkaline earth metal hydroxides (e.g., sodium hydroxide, potassium hydroxide, lithium hydroxide, barium hydroxide, etc.), alkali metal or alkaline earth metal carbonates (e.g., sodium carbonate, potassium carbonate, cesium carbonate, etc.), alkali metal or alkaline earth metal hydrogen carbonates (e.g., sodium hydrogen carbonate, potassium hydrogen carbonate, etc.), etc.; and

3) organic bases such as amines exemplified by triethylamine, diisopropylethylamine, N-methylmorpholine, dimethylaminopyridine, DBU (1,8-diazabicyclo[5.4.0]undec-7-ene), DBN (1,5-diazabicyclo[4.3.0]non-5-ene), etc., basic heterocyclic compounds exemplified by pyridine, imidazole, 2,6-lutidine, etc. Of these, preferred are triethylamine and 4-dimethylaminopyridine, etc.

ii) The Method Using a Reactive Derivative of Carboxy

The reactive derivative of Compound (III) and about 1 to about 5 equivalents (preferably about 1 to about 3 equivalents) of Compound (II) are reacted in an inert solvent.

The reactive derivatives of the “reactive derivative of Compound (III)” include acid halide (e.g., acid chloride, acid bromide, etc.), mixed acid anhydride (e.g., anhydride with C₁₋₆ alkyl carboxylic acid, C₆₋₁₀ aryl carboxylic acid or C₁₋₆ alkyl carbonic acid, etc.), activated ester (e.g., ester with phenol optionally having substituents, 1-hydroxybenzotriazole, 1-hydroxy-7-azabenzotriazole, 1-hydroxy-5-norbornen-2,3-dicarboxyimide or N-hydroxysuccinimide, etc.).

The “substituent” of the “phenol optionally having substituents” includes, for example, halogen atom, nitro, optionally halogenated C₁₋₆ alkyl, optionally halogenated C₁₋₆ alkoxy, etc. The number of the substituents is, for example, 1 to 5. The concrete examples of the “phenol optionally having substituents” include phenol, pentachlorophenol, pentafluorophenol, p-nitrophenol, etc. The reactive derivative is preferably acid halide.

The “inert solvent” includes, for example, ethers, halogenated hydrocarbons, aromatic solvents, nitriles, amides, ketones, sulfoxides, water, esters, etc. These may be used on mixing two or more kinds at a suitable proportion. Of these, preferred are tetrahydrofuran (THF), acetonitrile, dichloromethane, chloroform, ethyl acetate, etc.

The reaction temperature is generally about −20° C. to about 50° C., preferably at room temperature.

The reaction time is generally about 5 minutes to about 40 hours, preferably about I to about 5 hours.

In the present reaction, about 1 to about 10 equivalents, preferably about 1 to about 3 equivalents of a base is used if necessary.

As said “base”, those exemplified in the above-mentioned “method using a dehydrating/condensing agent” are used. Among them, preferred are sodium hydride, potassium carbonate, sodium carbonate, sodium hydroxide, potassium hydroxide, sodium hydrogen carbonate, potassium hydrogen carbonate, triethylamine, pyridine, etc.

In the present reaction, about 0.1 to about 1 equivalent, preferably about 0.1 to about 0.5 equivalent of a phase-transfer catalyst is used where necessary.

Said “phase-transfer catalyst” includes, for example, quaternary ammonium salt such as tetrabutylammonium hydrogensulfate, benzyltriethylammonium chloride, etc. Of these, preferred is tetrabutylammonium hydrogensulfate.

Moreover, when a functional group adjacent to the leaving group L¹ is CONR⁷ (R⁷ is as defined above) or COO for Y in compound (III), the present process is conducted by urea formation and carbamoylation.

Said urea formation and carbamoylation are conducted by, for example, reacting compound (II) with 1 to 2 equivalents of the compound represented by the formula: L²-CO-L³ (VIII) wherein L² and L³ represent leaving group, in an inert solvent at room temperature for about 0.5 to 5 hours, and then reacting the obtained compound with 1 to 2 equivalents of the compound represented by the formula: H—Yb—Yc-Z (IX) wherein Yb represents NR⁷ (R⁷ is as defined above) or oxygen atom, Yc represents a spacer having a main chain of 1 to 5 atoms and Z is as defined above, in an inert solvent at room temperature for about 0.5 to 24 hours.

The “leaving group” represented by L² or L³ is exemplified by one mentioned as the above L¹. Of these, preferred are chlorine and succinimidoxy, and succinimidoxy is specifically preferred.

The “spacer having a main chain of 1 to 5 atoms”, which is represented by Yc, is exemplified by the above-mentioned “spacer having a main chain of 1 to 6 atoms” exemplified for Y, which has a main chain of 1 to 5 atoms.

The “inert solvent” includes, for example, nitrites, ethers, halogenated hydrocarbons, etc. These may be used on mixing two or more kinds at a suitable proportion. Of these, preferred are acetonitrile, THF, dichloromethane, etc.

In the present reaction, about I to about 5 equivalents of a base (e.g., N-ethyldiisopropylamine, etc.) is also used where necessary.

Moreover, when a functional group adjacent to the leaving group L¹ is non-carbonyl carbon atom for Y in compound (III), the present reaction is conducted by alkylation.

Said alkylation is conducted by, for example, reacting compound (II) with about 1 to about 5 equivalents (preferably about 1 to about 2 equivalents) of compound (III) in an inert solvent in the presence of a base.

As said “base”, those exemplified in the above-mentioned Process 1 are used. Of those, preferred are potassium carbonate, sodium hydride, potassium hydroxide, etc. The amount of the base used is, for example, about 1 to about 5 equivalents of compound (II).

The “inert solvent” includes, for example, alcohols, ethers, halogenated hydrocarbons, aromatic solvents, nitriles, amides, ketones, sulfoxides, water, etc. These may be used on mixing two or more kinds at a suitable proportion. Of these, preferred are acetonitrile, N,N-dimethyl formamide (DMF), acetone, ethanol, pyridine, water, etc.

The reaction temperature is generally −20° C. to 100° C., preferably at room temperature to 80° C.

The reaction time is generally 0.5 hour to 1 day.

Moreover, when a functional group adjacent to the leaving group L¹ is methylene group for Y in compound (III), the present process can be conducted by subjecting the compound (II) and the compound represented by the formula: OHC-Yc-Z (X) wherein each symbol is as defined above to a reductive alkylation.

Said reductive alkylation can be conducted by methods known per se, for example, by reacting compound (II) and about 1 to about 5 equivalents (preferably 1 to 2 equivalents) of the compound (X) in an inert solvent in the presence of metal hydride.

The “metal hydride” includes, for example, aluminum hydride, lithium aluminum hydride, sodium borohydride, lithium borohydride, sodium cyanoborohydride, lithium cyanoborohydride, sodium triacetoxyborohydride, borane complexes (e.g., borane-THF complex, catechol-borane, etc.), dibutyl aluminum hydride, etc. These metal hydrides may be used on mixing with Lewis acids (e.g., aluminum chloride, titanium tetrachloride, cobalt chloride, etc.) or phosphorus oxychloride at a suitable proportion. The metal hydride is preferably sodium cyanoborohydride, sodium triacetoxyborohydride, etc.

The amount of the metal hydride used is, for example, generally about 1 to 5 equivalents of compound (II).

The “inert solvent” includes, for example, alcohols (preferably ethanol), ethers (preferably THF), nitrites (preferably acetonitrile), acetic acid, etc. These may be used on mixing two or more kinds at a suitable proportion.

The amount of the compound (X) used is, for example, about 1 to 5 equivalents, preferably 1 to 2 equivalents, of the compound (II).

The reaction temperature varies depending on the kind of metal hydride used, but is generally about −70° C. to 100° C., preferably at room temperature to 80° C.

The reaction time is generally about 0.1 hour to 48 hours.

The above compound (II), compound (III), compound (VIII), compound (IX) and compound (X) can be produced by methods known per se. For example, the compound (II) can be produced by the methods described in Tetrahedron Letters, 39, 3445 (1998); Tetrahedron Letters, 39, 8729 (1998), etc. or a similar method thereto, etc.

Process 2: deprotection

In the present process, compound (V) can be produced by deprotecting the compound (IV). The present reaction can be carried out by methods known per se according to the kind of R which is a protective group of carboxyl group.

Of the thus-obtained compounds (V), a compound wherein Z is Zb (Zb is piperidinyl or piperazinyl, each of which is substituted by a group of the formula: -Yd-Ara (Yd and Ara are as defined above)) is a novel compound.

Process 3: amidation

In the present process, compound (Ia) can be produced by reacting compound (V) with compound (VI).

The present reaction is conducted in the same manner as the amidation in the above-mentioned Process 1.

The above-mentioned compound (VI) can be produced by methods known per se.

Process 4: Induction of the group represented by the formula: -Ya-Za (wherein each symbol is as defined above)

In the present process, compound (I) can be produced by reacting compound (Ia) with compound (VII).

The present reaction is conducted in the same manner as the above-mentioned Process 1.

The above-mentioned compound (VII) can be produced by methods known per se. 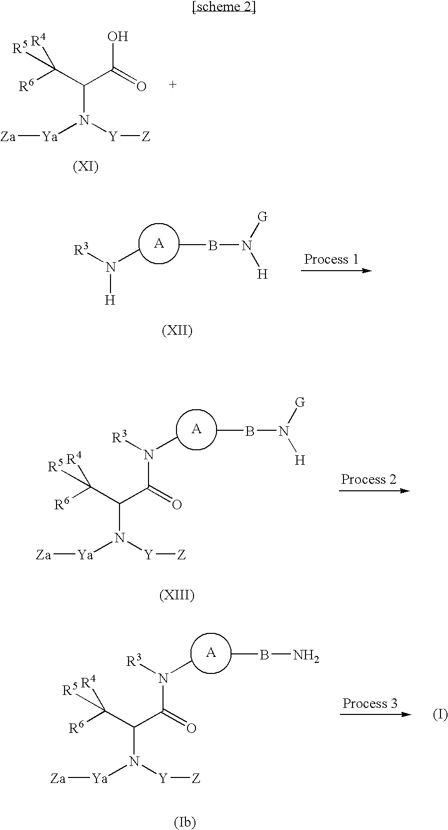
wherein G represents a protective group of amino, and the other symbols are as defined above.

The protective group of amino represented by G includes, for example, formyl, C₁₋₆ alkyl-carbonyl (e.g., acetyl, propionyl, etc.), C₁₋₆ alkoxy-carbonyl (e.g., methoxycarbonyl, ethoxycarbonyl, tert-butoxycarbonyl, etc.), benzoyl, C₇₋₁₀ aralkyl-carbonyl (e.g., benzylcarbonyl, etc.), C₇₋₁₄ aralkyloxy-carbonyl (e.g., benzyloxycarbonyl, 9-fluorenylmethoxycarbonyl, etc.), trityl, phthaloyl, N,N-dimethylaminomethylene, silyl (e.g., trimethylsilyl, triethylsilyl, dimethylphenylsilyl, tert-butyldimethylsilyl, tert-butyldiethylsilyl, etc.), C₂₋₆ alkenyl (e.g., 1-allyl, etc.), etc. These groups may be substituted by 1 to 3 halogen atom (e.g., fluorine, chlorine, bromine, iodine, etc.), C₁₋₆ alkoxy (e.g., methoxy, ethoxy, propoxy, etc.), nitro, etc. The protective group of amino is preferably trifluoroacetyl, tert-butoxycarbonyl, 9-fluorenylmethoxycarbonyl, etc.

Process 1: amidation

In the present process, compound (XIII) can be produced by reacting compound (XI) with compound (XII).

The present reaction is conducted in the same manner as the amidation in the above-mentioned Process 1 in the scheme 1.

Process 2: deprotection

In the present process, compound (Ib) can be produced by subjecting compound (XIII) to deprotection. The present reaction can be carried out by methods known per se according to the kind of G which is a protective group of amino group. Process 3: Induction of R¹ and R²

In the present process, compound (I) can be produced by subjecting compound (Ib) to alkylation.

The present reaction is conducted in the same manner as the alkylation or reductive alkylation in the above-mentioned Process 1 in the scheme 1.

The induction of R¹ and R² may be carried out by the same reaction or different reactions for each of them.

The above-mentioned compound (XII) can be produced by methods known per se.

The above-mentioned compound (XI) can be also produced, for example, according to the following method represented by scheme 3. 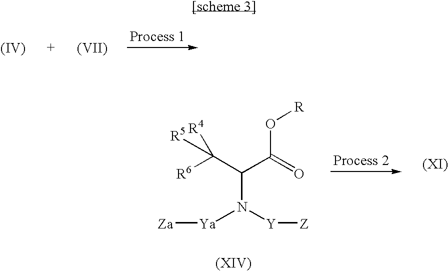
wherein each symbol is as defined above.
Process 1: Induction of the group represented by the formula: -Ya-Za (each symbol is as defined above)

In the present process, compound (XIV) can be produced by reacting compound (IV) with compound (VII).

The present reaction is conducted in the same manner as the above-mentioned Process 1 in the scheme 1.

Process 2: deprotection

In the present process, compound (XI) can be produced by subjecting compound (XIV) to deprotection. The present reaction can be carried out by methods known per se according to the kind of R which is a protective group of carboxyl group.

In the thus-obtained compound (I), functional groups in a molecule can be converted into the desired functional groups by combination of per se known chemical reactions. Examples of the chemical reactions include oxidation, reduction, alkylation, hydrolysis, amination, esterification, aryl-coupling reaction, deprotection, etc.

The above-mentioned “alcohols” includes, for example, methanol, ethanol, isopropanol, tert-butanol, etc.

The above-mentioned “ethers” includes, for example, diethyl ether, tetrahydrofuran (THF), 1,4-dioxane, 1,2-dimethoxyethane, etc.

The above-mentioned “halogenated hydrocarbons” includes, or example, dichloromethane, chloroform, 1,2-dichloroethane, carbon tetrachloride, etc.

The above-mentioned “aromatic solvents” includes, for example, benzene, toluene, xylene, pyridine, etc.

The above-mentioned “amides” includes, for example, N,N-dimethylformamide (DMF), N,N-dimethylacetamide, N-methylpyrrolidone, etc.

The above-mentioned “ketones” includes, for example, acetone, methylethylketone, etc.

The above-mentioned “sulfoxides” includes, for example, dimethylsulfoxide (DMSO), etc.

The above-mentioned “nitriles” includes, for example, acetonitrile, propionitrile, etc.

The above-mentioned “esters” includes, for example, ethyl acetate, etc.

In the above-mentioned reactions where the starting compounds are substituted by any of amino, carboxy, hydroxy or carbonyl, those groups may be protected by ordinary protective groups which are generally used in peptide chemistry, etc. The protective groups may be removed after the reaction, if necessary, to give the desired compounds.

The protective group of amino is exemplified by one mentioned as the above G.

The protective group of carboxyl group is exemplified by one mentioned as the above R.

The protective group of hydroxy includes, for example, C₁₋₆ alkyl (e.g., methyl, ethyl, propyl, isopropyl, butyl, tert-butyl, etc.), phenyl, trityl, C₇₋₁₀ aralkyl (e.g., benzyl, etc.), formyl, C₁₋₆ alkyl-carbonyl (e.g., acetyl, propionyl, etc.), benzoyl, C₇₋₁₀ aralkyl-carbonyl (e.g., benzylcarbonyl, etc.), 2-tetrahydropyranyl, 2-tetrahydrofuranyl, silyl (e.g., trimethylsilyl, triethylsilyl, dimethylphenylsilyl, tert-butyldimethylsilyl, tert-butyldiethylsilyl, etc.), C₂₋₆ alkenyl (e.g., 1-allyl, etc.), etc. These groups may be substituted by 1 to 3 halogen atoms (e.g., fluorine, chlorine, bromine, iodine, etc.), C₁₋₆ alkyl (e.g., methyl, ethyl, propyl, etc.),

C₁₋₆ alkoxy (e.g., methoxy, ethoxy, propoxy, etc.), or nitro, etc.

The protective group of carbonyl includes, for example, cyclic acetal (e.g., 1,3-dioxane, etc.), non-cyclic acetal (e.g., di-C₁₋₆ alkylacetal, etc.), etc.

Those protective groups may be removed by methods known per se, for example, the methods described in Protective Groups in Organic Synthesis, published by John Wiley and Sons, 1980, etc. For example, employed are the methods using acids, bases, ultraviolet ray, hydrazine, phenylhydrazine, sodium N-methyldithiocarbamate, tetrabutylammonium fluoride, palladium acetate, trialkylsilylhalide (e.g., trimethylsilyliodide, trimethylsilylbromide, etc.), etc.; and reduction, etc.

Compound (I) can be isolated and purified by any known procedures, for example, solvent extraction, pH adjustment, redistribution, crystallization, recrystallization, chromatography, etc.

The starting compounds for compound (I) (various compounds indicated in the above-mentioned schemes 1 and 2) can be isolated and purified according to the same known procedures as above. It is also possible to use as a raw material in the next step a reaction mixture containing these compounds without any isolation procedure.

The compound (I) may also be in the form of hydrates or non-hydrates thereof.

Where compound (I) includes optical isomers, stereo isomers, regio isomers and rotational isomers, those are within the scope of compound (I), and can be isolated as their single compound through synthesis or separation known per se. For example, where optical isomers of compound (I) exist, those resolved from their mixtures through optical resolution are within the scope of compound (I).

The optical isomers can be produced by methods known per se. Concretely, optically active synthetic intermediates may be used, or mixtures of racemate of the final product are subjected to ordinary optical resolution to give the corresponding optical isomers.

For the optical resolution, employable are methods known per se, such as a fractional recrystallization method, a chiral column method, a diastereomer method, etc.

1) Fractional Recrystallization Method

The method which comprises allowing a racemate to react with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine, etc.) to give a salt, which is then isolated through fractional recrystallization method, followed by, when desired, subjecting the isolated compound to neutralization to obtain free optical isomers.

2) Chiral Column Method

The method of separating a racemate or a salt thereof, which comprises utilizing a column for fractionating optical isomers (chiral column). In the case of liquid column chromatography, for example, a mixture of optical isomers is applied to a chiral column, such as ENANTIO-OVM (manufactured by Tosoh Corp.), CHIRAL SERIES (manufactured by Daicel Co.), etc., which is then eluted with water, various buffers (e.g., phosphate buffer) and organic solvents (e.g., ethanol, methanol, isopropanol, acetonitrile, trifluoroacetic acid, diethylamine, etc.), singly or as a suitable mixture of them, to isolate the individual optical isomers. In the case of gas chromatography, for example, a chiral column such as CP-Chirasil-DeX CB (manufactured by GL Science Co.), etc. is used for isolation.

3) Diastereomer Method

A racemic mixture is chemically reacted with an optically-active reagent to give a mixture of diastereomer, which is subjected to ordinary separation means (e.g., fractional recrystallization, chromatography, etc.) to give single compounds. The thus-isolated single compounds are then chemically processed, for example, through hydrolysis to thereby remove the optically-active reagent site from the compounds to obtain optical isomers. For example, where compound (I) has a hydroxy group or a primary or secondary amino group in the molecule, it is condensed with an optically-active organic acid (e.g., MTPA [α-methoxy-α-(trifluoromethyl)phenyl-acetic acid], (−)-menthoxyacetic acid, etc.) or the like to give the corresponding ester-type or amide-type diastereomer. On the other hand, where compound (I) has a carboxylic acid group, it is condensed with an optically active amine or alcohol reagent to give the corresponding amide-type or ester-type diastereomer. The thus-isolated diastereomer is then subjected to acidic or basic hydrolysis, through which it is converted into the optical isomer of the original compound.

Compound (I) has optical active centers at 2- and 3-positions in propanoyl group having substituent: R⁴, R⁵ and R⁶ at 3-position. In said optical active center, there exist (R)-isomer and (S)-isomer. Among those, preferred is (2R,3S) compound.

The compound (I) or a prodrug thereof [in the present specification sometimes to be abbreviated as the compound of the present invention] has an excellent somatostatin receptor binding inhibition activity (i.e., an activity to inhibit the binding of somatostatin to somatostatin receptors; specifically, a somatostatin receptor agonist activity and antagonist activity). The somatostatin receptor here includes somatostatin subtypes 1, 2, 3, 4, 5, etc. Especially, the compound of the present invention has a selective somatostatin subtype 2 receptor (SSTR2) binding inhibition activity, particularly a somatostatin subtype 2 receptor agonist activity.

The compound of the present invention acts through various intracellular signal transduction systems with which somatostatin is associated. The “intracellular signal transduction systems” include, for example, that which involves adenylate cyclase, K⁺ channels, Ca²⁺ channels, dephosphorylation of protein, phospholipase C/inositol trisphosphate production systems, MAP kinase, Na⁺/H⁺ exchanger, phospholipase A2, a transcription factor such as NF-κB. The compound of the present invention modulates a direct or indirect cell proliferation inhibitory action or apoptosis both of which are associated with somatostatin.

Further, the compound of the present invention is low in its toxicity, and enhances or inhibits production and/or secretion of a variety of hormones, growth factors and physiologically active substances, etc. by effecting on somatostatin receptors in mammals (e.g., human, cattle, horse, dog, cat, monkey, mouse and rat, especially, human).

The “hormones” include, for example, growth hormone (GH), growth hormone-releasing hormones (GHRH), ghrelin, thyroid stimulating hormone (TSH), prolactin, insulin, glucagon, etc. The “growth factors” include, for example, insulin-like growth factor-1 (IGF-1) and vascular endothelial cell growth factor (VEGF). Said- “physiologically active substances” include, for example, vasoactive intestinal polypeptide (VIP); gastrin; glucagon-like peptide-1 (GLP-1); glucose-dependent insulinotropic polypeptide (GIP); amylin; substance-P, CCK (cholecystokinin); amylase; interleukins such as interleukin-6 (IL-6), interleukin-l (IL-1), etc.; cytokines such as TNF-α, etc.; cardiotropin, etc.

Therefore, the compound of the present invention is safe, and useful for disorders of the above intracellular signal transduction systems (e.g., diseases accompanied by excess sthenia or suppression, etc.); diseases accompanied by disorders of regulating cell proliferation; diseases accompanied by disorders of production and/or secretion of hormones, growth factors, physiologically active substances, etc.; or facilitating growth, immune, gastroenteric or metabolic functions, etc; and the like.

For example, the compound of the present invention is useful (1) for drugs for treatment of tumors such as acromegaly, TSH-producing tumors, nonsecretory (afunctional) hypophysial tumors, ectopic ACTH (adrenocorticotrophic hormone)-producing tumors, medullar thyroid carcinoma, VIP-producing tumors, glucagon-producing tumors, gastrin-producing tumors, insulinoma and carotinoid, (2) for drugs for treatment of diabetes such as insulin-dependent (type I) and non-insulin dependent (type II) diabetes mellitus or a variety of diseases associated with them, namely, diabetic complications such as diabetic retinopathy, diabetic nephropathy, diabetic neuropathy, Doan syndrome and orthostatic hypotension, (3) for drugs for treatment of obesity or overeating, etc. by improvement of hyperinsulinemia or inhibition of appetite, etc. (4) for drugs for treatment of acute pancreatitis, chronic pancreatitis, pancreal/intestinal fistula, hemorrhagic ulcer, peptic ulcer, gastritis, hyperchylia, regurgitant esophagitis, etc. (5) for drugs for improvement of various symptoms accompanied by the Helicobacter pylori infection, for example, inhibitors of gastrin hypersecretion, etc. (6) for drugs for inhibition of amylase secretion accompanied by endoscopic cholangiopancreatography, and drugs for prognostic treatment of surgical operation of pancreas, (7) for drugs for treatment of diarrhea due to intestinal malabsorption, promotion of secretion or dyskinesia of the digestive tracts (for example, short bowel syndrome, etc.), diarrhea due to the drugs for cancer chemotherapy, diarrhea due to congenital small intestine atrophy, diarrhea due to neuroendocrine tumors such as VIP-producing tumors, diarrhea due to AIDS, diarrhea due to graft versus host reaction accompanied by bone marrow transplantation, diarrhea due to diabetes mellitus, diarrhea due to celiac plexus blocking, diarrhea due to systemic sclerosis and diarrhea due to eosinophilia, etc. (8) for drugs for treatment of dumping syndrome, irritable colitis, Crohn disease and inflammatory bowel disease, etc. (9) for drugs for treatment of tumors or cancers (e.g., thyroid cancer, colon cancer, breast cancer, prostatic cancer, small cell lung cancer, non-small cell lung cancer, pancreatic cancer, stomach cancer, cholangiocarcinoma, hepatic cancer, vesical cancer, ovarian cancer, melanoma, osteosarcoma, chondrosarcoma, malignant pheochromocytoma, neuro-blastoma, brain tumors, thymoma, renal cancers, etc.), leukemia (e.g., leukemia of basophilic leukocyte, chronic lymphocytic leukemia, chronic myeloid leukemia, Hodgkin disease, and non-Hodgkin lymphoma, etc.); the drugs can be used for monotherapy or concomitant therapy with other anticancer drugs such as Tamoxifen, LHRH agonists, LHRH antagonists, interferon-α, β and γ, interleukin-2, etc.), (10) for drugs for prevention or treatment of hypertrophic cardiomyopathy, arteriosclerosis, valvular disease, myocardiac infarction (especially, myocardiac infarction post percutaneous transluminal coronary arterioplasty) and reangioplasty, (11) for drugs for treatment of hemorrhage of esophageal varicosis, cirrhosis and peripheral blood vessel disorders, (12) for drugs for treatment of diseases accompanied by general or local inflammation, for example, polyarteritis, rheumatoid arthritis, psoriasis, sunburn, eczema and allergy (e.g., asthma, atopic dermatitis and allergic rhinitis, etc.), because they modulate the secretion of physiologically active substances acting on the immune system (e.g., Substance P, tachykinin and cytokines), (13) for drugs for treatment of dementia (e.g., Alzheimer's disease, Alzheimer-type senile dementia, vascular/multi-infarct dementia, etc.), schizophrenia, epilepsy, depression, generalized anxiety disorder, sleep disorder, and multiple sclerosis, because they give influence on the production or secretion of nerve regulators, (14) for drugs for treatment of oculopathy (e.g., glaucoma, etc.), (15) for drugs for prevention or treatment of acute bacterial meningitis, acute virus encephalitis, adult respiratory distress syndrome, bacterial pneumonia, severe systemic mycotic infection, tuberculosis, spinal damage, bone fracture, hepatic failure, pneumonia, alcoholic hepatitis, virus A hepatitis, virus B hepatitis, virus C hepatitis, AIDS infection, human papilloma virus infection, influenza infection, metastasis of cancer, multiple myeloma, osteomalacia, osteoporosis, bone Paget disease, nephritis, renal failure, sepsis, septic shock, hypercalcemia, hypercholesterolemia, hypertriglyceridemia, hyperlipemia, systemic lupus erythematosus, transient ischemic attack and alcoholic hepatitis, etc., (16) for cure of organ transplantation, burns, trauma, and alopecia, etc. (17) as analgesics to suppress or relieve chronic or acute pain (e.g., postoperative pain, inflammatory pain, dental pain, bone disease (e.g., arthritis, rheumatism, osteoporosis, etc.) derived pain). Further, the compound of the present invention is useful for imaging of tumors having somatostatin receptors after introducing radioactive substance (e.g., ¹²³I, ¹²⁵I, ¹¹¹In, etc.) to the compound of the present invention directly or via a suitable spacer, and for targeting tumors having somatostatin receptors after introducing anti-cancer drugs to the compound of the present invention directly or via a suitable spacer.

Somatostatin is associated with secretion of hormone such as growth hormone, gastrin, glucagon (especially in the case of SSTR2), and therefore, when the compound of the present invention has somatostatin receptor antagonist activity, the compound of the present invention can be used for the purpose of promoting secretion of these hormones. Thus, the compound of the present invention can be used for the prevention or treatment of diseases or symptoms caused by insufficiency of growth-hormone or IGF-1.

The “prevention or treatment of diseases or symptoms caused by insufficiency of growth hormone or-IGF-1” includes, for example, treatment of diabetes such as insulin-dependent (type I) and non-insulin dependent (type II) diabetes mellitus or a variety of diseases associated with them, namely diabetic complications such as diabetic retinopathy, diabetic nephropathy, diabetic neuropathy, Doan syndrome and orthostatic hypotension, etc.; prevention of adverse effects caused by disassimilation of glucocorticoid; prevention or treatment of osteoporosis; stimulation of immune system (e.g., promotion of increase in hemocytes such as lymphocyte; strengthening of an antimicrobial activity or an antiviral activity); promotion of cure of burns and trauma; acceleration in the treatment of bone fracture; treatment of acute or chronic renal diseases; treatment or improvement of diseases or symptoms (short stature, delayed growth) associated with insufficiency of growth hormone in adults or infants; treatment of obesity; promotion of recovery after surgical operations; improvement of delayed growth associated with Prader-Willi syndrome and Turner's syndrome; treatment of delayed intrauterine growth and skeletogenous disorders; treatment of peripheral neuropathy; treatment of Noonan's syndrome, schizophrenia and depression; treatment or prevention of neurodegenerative diseases such as Alzheimer's disease and Parkinson's disease; treatment of pulmonary insufficiency and ventilation dependence; treatment of malabsorption syndrome; improvement of cachexia or protein loss caused by cancer or AIDS; promotion of weight increase or proteopexis in patients in the case of TPN (total parenteral nutrition); treatment of hyperinsulinemia; promotion of induction of ovulation; improvement of menopausal disorders; improvement of senile constitution (e.g., increase in bone mass; enhancement of motor function; improvement of kidney function and cardiac function; increase in motor function and mental activity, etc.);, treatment of heart diseases (e.g., cardiac muscle hypertrophy in cardiac failure, improvement of cardiac function, increase in the amount of cardiac muscle in congestive cardiomyopathy, etc.), etc.

Further, the compound of the present invention is useful in mammals such as domestic animals for promotion of growth; increase in milk production; strengthening of an antimicrobial or antiviral activity by stimulation of immune system; stimulation in growth of wool in sheep.

The compound of the present invention is especially useful as a prophylactic or therapeutic agent for diabetes or diabetic complications.

As mentioned above, since the compound of the present invention has a selective SSTR2 binding inhibition activity (preferably agonist activity), it is useful as a prophylactic or therapeutic agent for diabetes or diabetic complications (preferably diabetic nephropathy) without side effects based n its superior glucagon secretion inhibitory activity.

Moreover, the compound of the present invention is superior in metabolic stability and can exhibit efficacy in a sustained manner.

The compound of the present invention can be used with various concomitant drugs.

Examples of the concomitant drugs include a “agents for treating diabetes”, “agents for treating diabetic complications”, “agents for treating obesity”, “agents for treating hypertension”, “agents for treating hyperlipidemia”, “agents for treating arthritis”, “antianxiety agents”, “antidepressants”, “agents for treating osteoporosis”, “anticonvulsants”, “chemotherapeutic agents”, “immunotherapeutic drugs”, “antithrombotic drugs”, “antidementia drugs”, “erectile dysfunction ameliorating drugs”, “therapeutic agents for incontinentia and/or pollakiuria”, “therapeutic agents for dysuria”, “nonsteroidal anti-inflammatory drugs”, “local anesthetic”, “vitamins”, etc. Two or more kinds of these concomitant drugs can be combined in an appropriate ratio for use. In addition, these concomitant drugs may be low molecular weight compounds and may be macromolecules such as protein, polypeptide or antibody, or vaccine, etc.

Examples of the above “agents for treating diabetes” include insulin sensitizers, insulin secretagogues, biguanides, insulins, α-glucosidase inhibitors, β3 adrenaline receptor agonists, dipeptidylpeptidase IV inhibitors, amyrin agonist, phosphotyrosine phosphatase inhibitors, gluconeogenesis inhibitors, SGLT (sodium-glucose cotransporter) inhibitors, etc.

Examples of the insulin sensitizers include pioglitazone or its salt (preferably hydrochloride), rosiglitazone or its salt (preferably maleate), Reglixane (JTT-501), GI-262570, Netoglitazone (MCC-555), YM-440, DRF-2593, BM-13.1258, KRP-297, R-119702, CS-011, FK-614, compounds described in WO99/58510 (e.g., (E)-4-[4-(5-methyl-2-phenyl-4-oxazolylmethoxy)benzyloxyimino]-4-phenylbutyric acid), compounds described in WO01/38325, Tesaglitazar (AZ-242), Ragaglitazar (NN-622), BMS-298585, ONO-5816, LM-4156, MBX-102, LY-519818, MX-6054, LY-510929, Balaglitazone (NN-2344), T-131 or a salt thereof, THR-0921, etc.

Examples of the insulin secretagogues include sulfonylureas. Concrete examples of the sulfonylureas include tolbutamide, chlorpropamide, tolazamide, acetohexamide, glyclopyramide and its ammonium salt, glibenclamide, gliclazide, glimepiride, glipizide, glybuzole, etc.

Other than the above, examples of the insulin secretagogues include repaglinide, nateglinide, mitiglinide (KAD-1229), JTT-608, etc.

Examples of the biguanides include metformin, buformin, phenformin.

Examples of the insulins include animal insulins extracted from bovine or porcine pancreas; semi-synthetic human insulin which is enzymatically synthesized from insulin extracted from porcine pancreas; human insulin synthesized by genetic engineering, using Escherichia Coli or yeast. As insulin, also employed are insulin-zinc containing 0.45 to 0.9 (w/w) % of zinc; protamine-insulin-zinc produced from zinc chloride, protamine sulfate and insulin; etc. In addition, insulin can be an insulin fragment or derivative (e.g., INS-1, etc.), oral insulin preparation, etc.

Insulin can also include various types such as ultra immediate action type, immediate action type, two-phase type, intermediate type, prolonged action type, etc., and these can be selected depending on the pathological conditions of patients.

Examples of the a-glucosidase inhibitors include acarbose, voglibose, miglitol, emiglitate, etc.

Examples of the β3 adrenaline receptor agonists include AJ-9677, BMS-196085, SB-226552, AZ40140, CL-316243, SR-58611-A, UL-TG-307, etc.

Examples of the dipeptidylpeptidase IV inhibitors include NVP-DPP-278, PT-100, NVP-DPP-728, LAF237, P32/98, TS-021, etc.

Examples of the amyrin agonist include pramlintide, etc.

Examples of the phosphotyrosine phosphatase inhibitors include vanadic acid, etc.

Examples of the gluconeogenesis inhibitors include glycogen phosphorylase inhibitors, glucose-6-phosphatse inhibitors, glucagon antagonists, etc.

Examples of the SGLT (sodium-glucose cotransporter) inhibitors include T-1095, etc.

Other than the above, examples of the “agents for treating diabetes” include ergoset, leptin, BAY-27-9955, GLP-1, Exendine-4, GPR40 agonists, 11β-hydroxysteroid dehydrogenase inhibitors (e.g., BVT-3498, etc.), adiponectin or an agonist thereof, IKK inhibitors (e.g., AS-2868, etc.), leptin resistance improving drugs, somatostatin receptor agonists (compounds described in WO01/25228 and WO03/42204, compounds described in WO98/44921, WO98/45285 and WO99/22735, etc.), glucokinase activators (e.g., Ro-28-1675), etc.

Examples of the above “agents for treating diabetic complications” include aldose reductase inhibitors, glycation inhibitors, protein kinase C inhibitors, neurotrophic factors, neurotrophin increasing drugs, nerve regeneration stimulators, etc.

Examples of the aldose reductase inhibitors include torulestat; eparlestat; imirestat; zenarestat; SNK-860; zopolrestat; ARI-509; AS-3201, etc.

Examples of the glycation inhibitors include pimagedine, ALT946, pyradoxatine, N-phenacylthiazolium bromide (ALT766), EXO-226, etc.

Examples of the protein kinase C inhibitors include LY-333531, etc.

Examples of the neurotrophic factors include, for example, NGF, NT-3, BDNF, etc.

Examples of the neurotrophin increasing drugs include, for example, a neurotrophin production/secretion promoting agent (e.g., 4-(4-chlorophenyl)-2-(2-methyl-1-imidazolyl)-5-[3-(2-methylphenoxy)propyl]oxazole, etc.) described in WO01/14372, etc.

Examples of the nerve regeneration stimulators include Y-128, VX-853, prosaptide, etc.

Other than the above, examples of the “agents for treating diabetic complications” include alprostadil, thiapride hydrochloride, cilostazol, mexiletine hydrochloride, ethyl eicosapentate, memantine, pimagedline (ALT-711), AGE inhibitors (e.g., ALT946, pimagedine, pyradoxatine, N-phenacylthiazolinium bromide (ALT766), ALT-711, EXO-226, Pyridorin, pyridoxamine, etc.), active oxygen scavengers (e.g., thioctic acid, etc.), somatostatin receptor agonists (e.g., BIM23190), apoptosis signal-regulating kinase-1 (ASK-1) inhibitors, etc.

Examples of the above “agents for treating obesity” include pancreatic lipase inhibitors, anti-obesity drugs acting on the central nervous system, anorectic peptides, cholecystokinin agonists, etc.

Examples of the pancreatic lipase inhibitors include orlistat, ALT-962, etc.

Examples of the anti-obesity drugs acting on the central nervous system include mazindol, dexfenfluramine, fluoxetine, sibutramine, baiamine, fenfluramine, phentermine, amfepramone, dexamphetamine, phenylpropanolamine, clobenzorex, etc.

Examples of the anorectic peptides include leptin, CNTF (Ciliary Neurotrophic Factor), etc.

Examples of the cholecystokinin agonists include lintitript, FPL-15849, etc.

Other than the above, examples of the “agents for treating obesity” include lipstatin, MCH receptor antagonists (e.g., SB-568849; SNAP-7941; compounds encompassed in WO01/82925 and WO01/87834, etc.), neuropeptide Y antagonists (e.g., CP-422935, etc.), cannabinoid receptor antagonists (e.g., SR-141716, SR-147778, etc.), ghrelin antagonists, 11β-hydroxysteroid dehydrogenase. inhibitors (e.g., BVT-3498, etc.), β3 agonists (e.g., CL-316243, SR-58611-A, UL-TG-307, SB-226552, AJ-9677, BMS-196085, AZ40140, etc.), anorexigenic drugs (e.g., P-57, etc.), etc.

Examples of the above “agents for treating hypertension” include angiotensin converting enzyme inhibitors, calcium antagonists, potassium channel openers, angiotensin II antagonists, etc.

Examples of the angiotensin converting enzyme inhibitors include captopril, enarapril, alacepril, delapril (hydrochloride), lisinopril, imidapril, benazepril, cilazapril, temocapril, trandolapril, manidipine (hydrochloride), etc.

Examples of the calcium antagonists include nifedipine, amlodipine, efonidipine, nicardipine, etc.

Examples of the potassium channel openers include levcromakalim, L-27152, AL0671, NIP-121, etc.

Examples of the angiotensin II antagonists include losartan, candesartan cilexetil, valsartan, irbesartan, CS-866, E4177, 1-[[2′-(2,5-dihydro-5-oxo-4H-1,2,4-oxadiazol-3-yl)biphenyl-4-yl]methyl]-2-ethoxy-1H-benzimidazol-7-carboxylic acid, etc.

Examples of the above “agents for treating hyperlipidemia” include HMG-CoA reductase inhibitors, fibrate compounds, squalene synthase inhibitors, antioxidants, etc.

Examples of the HMG-CoA reductase inhibitors include pravastatin, simvastatin, lovastatin, atorvastatin, fluvastatin, lipantil, cerivastatin, itavastatin, ZD-4522, or their salts (e.g., sodium salts, calcium salts, etc.), etc.

Examples of the fibrate compounds include bezafibrate, clinofibrate, clofibrate, simfibrate, etc.

Examples of the squalene synthase inhibitors include compounds described in WO97/10224 (e.g., N-[[(3R,5S)-1-(3-acetoxy-2,2-dimethylpropyl)-7-chloro-5-(2,3-dimethoxyphenyl)-2-oxo-1,2,3,5-tetrahydro-4,1-benzooxazepin-3-yl]acetyl]piperidine-4-acetic acid, etc.), etc.

Examples of the above antioxidants include lipoic acid, probucol, etc.

Examples of the above “agents for treating arthritis” include ibuprofen, etc.

Examples of the above “antianxiety agents” include chlordiazepoxide, diazepam, oxazolam, medazepam, cloxazolam, bromazepam, lorazepam, alprazolam, fludiazepam, etc.

Examples of the above “antidepressants” include fluoxetine, fluvoxamine, imipramine, paroxetine, sertraline, etc.

Examples of the above “agents for treating osteoporosis” include bisphosphonates, vitamin D preparations, calcitonin preparations, PTH preparations, Osten, etc.

Examples of the above “anticonvulsants” include gabapentin, gabapentin MR agent, Trileptal, Keppra, Zonegran, Pregabalin, Harkoseride, carbamazepine, etc.

Examples of the above “chemotherapeutic agents” include alkylating agents (e.g., cyclophosphamide, ifosamide, etc.), metabolic antagonists (e.g., methotrexate, 5-fluorouracil or a derivative thereof, etc.), antitumor antibiotics (e.g., mitomycin, adriamycin, etc.), plant-derived antitumor agents (e.g., vincristine, vindesine, Taxol, etc.), cisplatin, carboplatin, etoposide, etc. Among these, 5-fluorouracil derivatives such as Furtulon and Neo-Furtulon are preferable.

Examples of the above “immunotherapeutic agents” include microorganism- or bacterium-derived components (e.g., muramyl dipeptide derivatives, Picibanil, etc.), immunopotentiator polysaccharides (e.g., lentinan, schizophyllan, krestin, etc.), cytokines produced by genetically engineering techniques (e.g., interferons, interleukins (IL), etc.), colony stimulating agents (e.g., granulocyte colony stimulating factor, erythropoietin, etc.), etc. Among these, interleukins such as IL-1, IL-2, IL-12 and the like are preferable.

Examples of the above “antithrombotic drugs” include heparin (e.g., heparin sodium, heparin calcium, dalteparin sodium, etc.), warfarin (e.g., potassium warfarin, etc.), antithrombin (e.g., aragatroban, etc.), thrombolytic agents (e.g., urokinase, tisokinase, alteplase, nateplase, monteplase, pamiteplase, etc.), platelet aggregation inhibitors (e.g., ticlopidine hydrochloride, cilostazol, ethyl icosapentaenoate, beraprost sodium, sarpogrelate hydrochloride, etc.), etc.

Examples of the above “antidementia drugs” include tacrine, donepezil, rivastigmine, galantamine, etc.

Examples of the above “erectile dysfunction ameliorating drugs” include apomorphine, sildenafil citrate, etc.

Examples of the above “therapeutic agents for incontinentia and/or pollakiuria” include flavoxate hydrochloride, oxybutynin hydrochloride, propiverine hydrochloride, etc.

Examples of the above “therapeutic agents for dysuria” include acetylcholine esterase inhibitors (e.g., distigmine), etc.

Examples of the above “nonsteroidal anti-inflammatory drugs” include aspirin, acetaminophen, indomethacin, etc.

Examples of the above “local anesthetic” include lidocaine, capsaicin, etc.

Examples of the above “vitamins” include vitamin B1, vitamin B12, etc.

Other than the above, the concomitant drugs include “hormones promoting other growth hormone secretion (e.g., GHRH), GH or IGF-1”, “cytokines or cytokine activity enhancing agents”, etc.

The time of administration of the above-mentioned concomitant drug are not limited, but the compound of the present invention and the concomitant drug can be administered simultaneously or at staggered times to the administration subject. The dose of the concomitant drug can be appropriately selected based on the dose which is clinically employed, and can be appropriately selected according to the administration subject, administration route, target disease, combination and the like.

The method for administrating concomitant drug is not limited as long as the compound of the present invention and the concomitant drug are combined at the time of administration. Examples of such methods include 1) administration of a single preparation prepared from the compound of the present invention and the concomitant drug at the same time; 2) concomitant administration of two kinds of preparations prepared from the compound of the present invention and the concomitant drug separately by the same administration route; 3) staggered administration of two kinds of preparations prepared from the compound of the present invention and the concomitant drug separately by the same administration route; 4) concomitant administration of two kinds of preparations prepared from the compound of the present invention and the concomitant drug separately by different administration routes; 5) staggered administration of two kinds of preparations prepared from the compound of the present invention and the concomitant drug separately by different administration routes (e.g., administration of the compound of the present invention and the concomitant drug in this order, or reverse order); and etc.

The proportion of the compound of the present invention and the concomitant drug can be appropriately selected according to the administration subject, administration route, target disease and the like.

When the compound of the present invention is used for the improvement of menopausal disorders, a hormone supplemental therapy (e.g., therapy by estrogen preparations, Raloxifene, Tamoxifen) can be used concomitantly.

A pharmaceutical preparation of the present invention can be produced according to a per se known method. Said pharmaceutical preparation can be produced by mixing the compound of the present invention and a pharmacologically acceptable carrier according to any per se known pharmaceutical manufacturing techniques.

The dosage forms of the pharmaceutical preparation of the present invention include, for example, oral preparations such as tablets (including sugar-coated tablets, film-coated tablets, sublingual tablets, orally disintegrating tablet), powders, granules, capsules (including soft capsules and microcapsules), troches, liquids (e.g., syrups, emulsions, suspensions), etc.; non-oral preparations such as injections (e.g., subcutaneous injection, intravenous injection, intramuscular injection, intraperitoneal injection, drip infusions, etc.), external application forms (e.g., transdermal preparations, ointments, etc.), suppositories, (e.g., rectal suppositories, vaginal suppositories, etc.), pellets, nasal preparations, transpulmonary agents (inhalant), eye drops, etc.; etc. These preparations may be controlled release preparations (e.g., sustained-release microcapsules, etc.) such as immediate-release preparations, sustained-release preparations, etc.

The compound of the present invention and the pharmaceutical preparation of the present invention can be safely administered orally or parenterally (e.g., by local, rectal and intravenous administration, etc.).

The content of the compound of the present invention in a pharmaceutical preparation of the present invention is 0.1 to 100 weight percent of the whole preparation.

The dose of the compound of the present invention and the pharmaceutical preparation of the present invention can be appropriately selected depending on the administration subject, administration route, disease, etc. For instance, when these are orally administered as a prophylactic or therapeutic agent for diabetes or diabetic complications to an adult patient (body weight: about 60 kg), the dose is about 0.1 to about 500 mg, preferably about 1 to about 100 mg, more preferably about 5 to about 100 mg, in terms of the compound of the present invention. These amounts can be divided into one to several doses per day for administration.

Examples of the pharmacologically acceptable carriers used for production of a pharmaceutical preparation of the present invention include various organic or inorganic carrier substances which are commonly used as materials for pharmaceutical preparations, such as excipients, lubricants, binders, and disintegrators in solid preparations; solvents, solubilizing agents, suspending agents, isotonizing agents, buffering agents, soothing agents, in liquid preparations. In addition, additives such as antiseptics, anti-oxidants, coloring agents, sweeteners, absorbents and moistening agents can be used, if necessary.

Examples of the excipients include lactose, sucrose, D-mannitol, starch, cornstarch, crystalline cellulose, light anhydrous silicic acid, etc.

Examples of the lubricants include magnesium stearate, calcium stearate, talc, colloidal silica, etc.

Examples of the binders include crystalline cellulose, sucrose, D-mannitol, dextrin, hydroxypropylcellulose, hydroxypropylmethylcellulose, polyvinylpyrrolidone, starch, saccharose, gelatin, methylcellulose, carboxymethylcellulose sodium, etc.

Examples of the disintegrators include starch, carboxymethylcellulose, carboxymethylcellulose calcium, crosscarmellose sodium, carboxymethylstarch sodium, L-hydroxypropylcellulose, etc.

Examples of the solvents include water for injection, alcohol, propylene glycol, macrogol, sesame oil, corn oil, etc.

Examples of the solubilizing agents include polyethylene glycol, propylene glycol, D-mannitol, benzyl benzoate, ethanol, trisaminomethane, cholesterol, triethanolamine, sodium carbonate, sodium citrate, etc.

Examples of the suspending agents include surfactants such as stearyltriethanolamine, sodium lauryl sulfate, lauryl amino propionic acid, lecithin, benzalkonium chloride, benzethonium chloride, glyceryl monostearate, etc.; or hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose, etc.

Examples of the isotonizing agents include glucose, D-sorbitol, sodium chloride, glycerin, D-mannitol, etc.

Examples of the buffering agents include buffer solutions of phosphate, acetate, carbonate and citrate, etc.

Examples of the soothing agents include benzyl alcohol, etc.

Examples of the antiseptics include paraoxybenzoates, chlorobutanol, benzyl alcohol, phenethyl alcohol, dehydroacetic acid, and sorbic acid, etc.

Examples of the anti-oxidants include sulfite, ascorbic acid, etc.

Examples of the coloring agents include a water-soluble edible tar pigments (e.g., edible pigments such as edible color Red No.2 and No.3, edible color Yellow No.4 and No.5, edible color Blue No.1 and No.2), a water-insoluble lake pigments (e.g., aluminum salts of the water-soluble edible tar pigments listed above), a natural pigment (e.g., β-carotene, chlorophyll, iron oxide red), etc.

Examples of the sweeteners include saccharin sodium, dipotassium glycyrrhizinate, aspartame, stevia, etc.

The pharmaceutical preparation of the present invention can be produced by the methods well established in fields of the pharmaceutical manufacturing techniques, for example by the methods described in the Japanese Pharmacopoeia. In the following, some typical methods for producing such preparations are described in detail.

An oral preparation, for instance, is produced by compression molding a mixture prepared by adding, to the active ingredient, an excipient (e.g., lactose, sucrose, starch, D-mannitol), a disintegrator (e.g., carboxymethyl cellulose calcium), a binder (e.g., hydroxypropyl cellulose, polyvinyl pyrrolidone) or a lubricant (e.g., talc, magnesium stearate), for instance, if necessary followed by coating by a per se known method using a coating base for attaining taste masking, enteric coating or sustained release.

Examples of the coating base include a sugar coating base, a water-soluble film coating base, an enteric film coating base, a sustained-release film coating base, etc.

Useful as the sugar coating base is sucrose and, further, one or more ingredients selected from talc, precipitated calcium carbonate, gelatin, gum arabic, pullulan, carnauba wax and the like may be used in combination.

Examples of the water-soluble film coating base include cellulose polymers such as hydroxypropylcellulose, hydroxypropylmethylcellulose, hydroxyethylcellulose and methylhydroxyethylcellulose; synthetic polymers such as polyvinylacetal diethylaminoacetate, aminoalkyl methacrylate copolymer E (Eudragit E (trademark), Rhom Pharma] and polyvinylpyrrolidone; and polysaccharides such as pullulan.

Examples of the enteric film coating base include cellulose polymers such as hydroxypropylmethylcellulose phthalate, hydroxypropylmethylcellulose acetate succinate, carboxymethylethylcellulose, and cellulose acetate phthalate; acrylic acid polymers such as methacrylic acid copolymer L [Eudragit L (trademark), Rhom Pharma], methacrylic acid copolymer LD [Eudragit L-30D55 (trademark), Rhom Pharma] and methacrylic acid copolymer S [Eudragit S (trademark), Rhom Pharma]; and natural products such as shellac and the like.

Examples of the sustained-release film coating base include cellulose polymers such as ethylcellulose; acrylic acid polymers such as aminoalkyl methacrylate copolymer RS [Eudragit RS (trademark), Rhom Pharma] and an ethyl acrylate-methyl methacrylate copolymer suspension [Eudragit NE (trademark), Rhom Pharma]; and so forth.

Two or more of the above coating bases may be used in admixture in appropriate proportions. On the occasion of coating, a shading agent such as titanium oxide, red ferric oxide may be used.

Injections are produced by dissolving, suspending or emulsifying the active ingredient in an aqueous solvent (e.g. distilled water, physiological saline, Ringer's solution) or an oleaginous solvent (e.g. vegetable oils such as olive oil, sesame oil, cotton seed oil, corn oil; propylene glycol), together with a dispersant (e.g. polysorbate 80, polyoxyethylene-hardened castor oil 60, polyethylene glycol, carboxymethylcellulose, sodium alginate), a preservative (e.g. methylparaben, propylparaben, benzyl alcohol, chlorobutanol, phenol), an isotonizing agent (e.g. sodium chloride, glycerol, D-mannitol, D-sorbitol, glucose) and the like. If desirable, additives such as a solubilizing agent (e.g. sodium salicylate, sodium acetate), a stabilizer (e.g. human serum albumin), a soothing agent (e.g. benzyl alcohol), may be used.

A compound represented by the formula (I), wherein R³ is linked with ring A together with the adjacent nitrogen atom to form 5- to 7-membered nitrogen-containing heterocyclic ring, a salt thereof and a prodrug thereof may be produced in the same manner as compound (I), have the same somatostatin receptor binding inhibition activity as does compound (I), and are used as prophylactic or therapeutic agents for various diseases such as diabetes, etc.

As used herein, the salt and the prodrug thereof are exemplified by those mentioned with regard to compound (I).

As the “5- to 7-membered nitrogen-containing heterocyclic ring” formed by R³ linked with ring A together with the adjacent nitrogen atom, those exemplified for the above-mentioned “5- to 7-membered nitrogen-containing heterocyclic ring” formed by R¹ linked with ring A together with the adjacent nitrogen atom and B can be mentioned. Of these, preferred are morpholine, thiomorpholine, piperidine, piperazine, pyrrolidine, etc.

The present invention is described in detail by way of the following Reference Examples, Examples, Formulation Examples and Experimental Examples. These are not intended to restrict the present invention, and may be modified within the range not deviating from the scope of this invention.

The “room temperature” in the following Reference Examples and Examples means a temperature of 0° C. to 30° C. For drying an organic layer, anhydrous magnesium sulfate or anhydrous sodium sulfate was employed. Unless otherwise specifically indicated, “%” means percent by weight. The solvent ratio when a mixed solvent is used is a volume ratio.

The mass spectrum was measured by ESI.

The meanings of the abbreviations used in the present specification are as follows:
s: singlet
d: doublet
t: triplet
m: multiplet
J: coupling constant
Hz: hertz
CDCl₃: deuterated chloroform
DMSO-d₆: deuterated dimethylsulfoxide
THF: tetrahydrofuran
DMF: N,N-dimethyl formamide
DME: 1,2-dimethoxyethane
DBU: 1,8-diazabicyclo[5.4.0]undec-7-ene
WSC: 1-ethyl-3-(3-dimethylaminopropyl) carbodiimide hydrochloride
HOBt: 1-hydroxy-1H-benzotriazol monohydrate
IPE: diisopropyl ether
Me: methyl
Et: ethyl

¹H-NMR: proton nuclear magnetic resonance spectrum (generally measured as the free form of each sample in CDCl₃)

### EXAMPLES

### Reference Example 1

### methyl (2R,3S)-3-(1H-indol-3-yl)-2-(((4-phenyl-1-piperidinyl)carbonyl)amino)butanoate

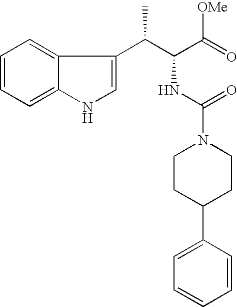

To a solution of methyl (2R,3S)-2-amino-3-(1H-indol-3-yl)butanoate (325 mg) and diisopropylethylamine (0.293 mL) in acetonitrile (10 mL) was added N,N′-disuccinimidyl carbonate (390 mg) under ice-cooling and the mixture was stirred for 1 hour. To the obtained solution was added a solution of 4-phenylpiperidine hydrochloride (332 mg) and DBU (0.252 mL) in acetonitrile (1 mL) and diisopropylethylamine (0.293 mL) under ice-cooling. The reaction solution was stirred at room temperature for 16 hours, and a saturated aqueous solution of sodium hydrogen carbonate was added. The mixture was extracted with ethyl acetate. The extract was purified by silica gel column chromatography (developing solvent; ethyl acetate) to give the title compound as a colorless amorphous powder (0.66 g, yield 100%).

¹H NMR (300 MHz, CDCl₃) δ ppm: 1.50 (d, J=7.3 Hz, 3 H), 1.54-1.69 (m, 2 H), 1.77-1.81 (m, 2 H), 2.57-2.67 (m, 1 H), 2.73-2.88 (m, 2 H), 3.62 (s, 3 H), 3.64-3.71 (m, 1 H), 3.89-3.95 (m, 1 H), 4.01-4.08 (m, 1 H), 4.83 (dd, J=8.1, 5.4 Hz, 1 H), 5.01 (d, J=8.8 Hz, 1 H), 7.02 (d, J=2.4 Hz, 1 H), 7.07-7.22 (m, 5 H), 7.27-.7.36 (m, 3 H), 7.62 (d, J=7.3 Hz, 1 H), 8.14 (s, 1 H).

### Reference Example 2

### (2R,3S)-3-(1H-indol-3-yl)-2-(((4-phenyl-1-piperidinyl)carbonyl)amino)butanoic acid

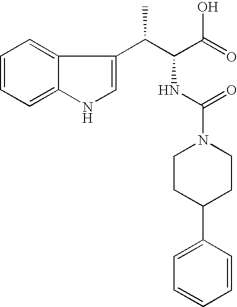

To a solution of methyl (2R,3S)-3-(1H-indol-3-yl)-2-(((4-phenyl-1-piperidinyl)carbonyl)amino)butanoate (0.65 g) in methanol (10 mL) was added an aqueous solution of 2N sodium hydroxide (2 mL) at room temperature and the mixture was stirred for 3 hours. The reaction solution was neutralized with 1N hydrochloric acid (4 mL) and extracted with ethyl acetate. The extract was dried (MgSO₄) and the solvent was removed by evaporation under reduced pressure. The residue was dissolved in methanol and the resulting solution was added dropwise to water with stirring. The resulting precipitates were collected by filtration and dried to give the title compound as a colorless amorphous powder (544 mg, yield 96%).

¹H NMR (300 MHz, DMSO-d6) δ ppm: 1.34 (d, J=6.9 Hz, 3 H), 1.40-1.53 (m, 2 H), 1.67-1.71 (m, 2 H), 2.62-2.79 (m, 3 H), 3.52-3.62 (m, 1 H), 4.10 (t, J=14.2 Hz, 2 H), 4.47 (t, J=7.6 Hz, 1 H), 6.28 (d, J=8.4 Hz, 1 H), 6.96 (t, J=6.9 Hz, 1 H), 7.04 (t, J=7.5 Hz, 1 H), 7.14-7.19 (m, 4 H), 7.26-7.33 (m, 3 H), 7.54 (d, J=8.1 Hz, 1 H), 10.81 (s, 1 H), 12.20 (s, 1 H).

### Reference Example 3

### methyl (2R,3S)-2-(((4-(4-fluorophenoxy)-1-piperidinyl)carbonyl)amino)-3-(1H-indol-3-yl)butanoate

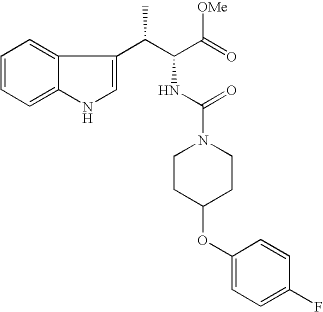

The title compound was obtained according to the same method as Reference Example 1.

¹H NMR (300 MHz, CDCl₃) δ ppm: 1.49 (d, J=7.3 Hz, 3 H), 1.65-1.76 (m, 2 H), 1.79-1.91 (m, 2 H), 3.14-3.29 (m, 2 H), 3.44-3.70 (m, 5 H), 4.31-4.38 (m, 1 H) 4.80 (dd, J=8.3, 5.1 Hz, 1 H), 4.98 (d, J=8.3 Hz, 1 H), 6.80-6.85 (m, 2 H), 6.93-7.02 (m, 3 H), 7.07-7.20 (m, 2 H), 7.3.5 (d, J=8.1 Hz, 1 H), 7.61 (d, J=7.3 Hz, 1 H), 8.11 (s, 1 H).

### Reference Example 4

### (2R,3S)-2-(((4-(4-fluorophenoxy)-1-piperidinyl)carbonyl)amino)-3-(1H-indol-3-yl)butanoic acid

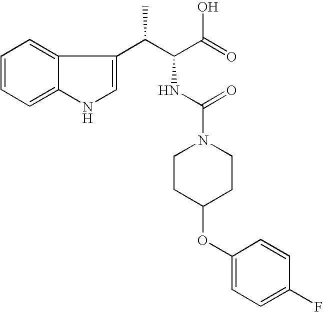

The title compound was obtained according to the same method as Reference Example 2.

¹H NMR (200 MHz, DMSO-d₆) δ ppm: 1.33 (d, J=7.0 Hz, 3 H), 1.36-1.56 (m, 3 H), 1.79-1.90 (m, 2 H), 3.03-3.20 (m, 2 H), 3.56-3.77 (m, 3 H), 4.45 (t, J=7.7 Hz, 2 H), 6.36 (d, J=8.4 Hz, 1 H), 6.92-7.16 (m, 7 H), 7.32 (d, J=7.3 Hz, 1 H), 7.54 (d, J=7.3 Hz, 1 H), 10.80 (s, 1 H), 12.12 (s, 1 H)

### Reference Example 5

### (2R,3S)-2-(((4-(4-fluorophenyl)-1-piperazinyl)carbonyl)amino)-3-(1H-indol-3-yl)butanoic acid

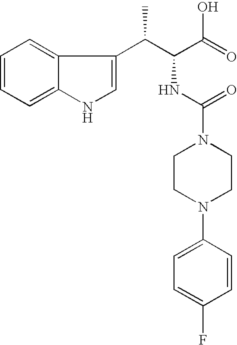

The title compound was obtained according to the same method as Reference Example 2.

¹H NMR (300 MHz, DMSO-d₆) δ ppm: 1.34 (d, J=7.2 Hz, 2 H), 3.00 (m, 4 H), 3.50 (m, 6 H), 4.46 (dd, J=8.5, 7.4 Hz, 1 H), 6.49 (d, J=8.7 Hz, 1 H), 7.00 (m, 5 H), 7.14 (d, J=2.3 Hz, 1 H), 7.31 (d, J=8.1 Hz, 1 H), 7.54 (d, J=7.9 Hz, 1 H), 10.81 (d, J=1.5 Hz, 1 H), 12.13 (s, 1 H).

### Reference Example 6

### 4-ethoxy-3-nitrobenzoic acid

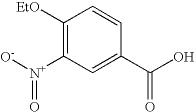

DMF (300 mL) was added to 4-hydroxy-3-nitrobenzoic acid (48.16 g, 0.26 mol) and potassium carbonate (128 g, 0.93 mmol). Ethyl iodide (100 mL, 1.25 mol) was added to the obtained suspension and the suspension was stirred at 90° C. for 1 hour. Ethyl iodide (50 mL, 0.63 mol) was further-added to the reaction solution and the mixture was stirred overnight, after which ethyl acetate and water were added and the mixture was extracted. The obtained organic layer was washed with 1N hydrochloric acid, dried over magnesium sulfate, and purified by silica gel column to give ethyl 4-ethoxy-3-nitrobenzoate.

Ethyl 4-ethoxy-3-nitrobenzoate was dissolved in a mixed solvent of THF (300 mL) and ethanol (200 mL). An aqueous solution of 2N sodium hydroxide (300 mL) was added to the obtained solution at room temperature. After stirring the reaction solution at room temperature for 3 days, 6N hydrochloric acid (120 mL) was added, and the solution was concentrated under reduced pressure to remove organic solvents. The resulting precipitates were collected by filtration, washed with water and dried to give the title compound (53.1.g, yield 96%).

¹H NMR (300 MHz, CDCl₃) δ ppm: 1.52 (t, J=7.1 Hz, 3 H), 4.28 (q, J=7. 0 Hz, 2 H), 7. 14 (d, J=9.0 Hz, 1 H), 8.2 5 (dd, J=8.9, 2.1 Hz, 1 H), 8.55 (d, J=2.1 Hz, 1 H).

### Reference Example 7

### 4-ethoxy-3-nitrobenzylalcohol

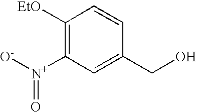

4-Ethoxy-3-nitrobenzoic acid (52.7 g, 0.25 mol) was added in small portions to 1.0 M borane-THF solution (500 mL, 0.50 mol) at room temperature, and the mixture was stirred overnight. A 1.0 M borane-THF solution (140 mL, 0.14 mol) was further added to the reaction solution, and the mixture was stirred at 60° C. for 2 hours. Methanol was added at room temperature until hydrogen production ceased and the mixture was concentrated. Ethyl acetate and 1N hydrochloric acid were added to the residue and the mixture was extracted. The organic layer was washed with saturated brine, dried over magnesium sulfate, filtered through a silica gel layer and concentrated. The obtained solid product was pulverized in hexane and diisopropyl ether, filtered, washed with hexane and dried to give the title compound (32.9 g, yield 67%).

¹H NMR (300 MHz, CDCl₃) δ ppm: 1.47 (t, J=7.0 Hz, 3 H), 1.75 (t, J=5.8 Hz, 1 H), 4.19 (q, J=7.0 Hz, 2 H), 4.69 (d, J=5.7 Hz, 2 H), 7.06 (d, J=8.7 Hz, 1 H), 7.52 (dd, J=8.6, 2.2 Hz, 1 H), 7.83 (d, J=2.1 Hz, 1 H).

### Reference Example 8

### 4-ethoxy-3-nitrobenzyl chloride

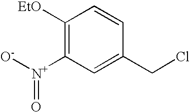

A suspension of 4-ethoxy-3-nitrobenzylalcohol (32.9 g, 0.17 mol) in toluene (120 mL) was added dropwise to thionyl chloride (30 mL, 0.41 mol), and the obtained mixed solution was reacted at room temperature for 30 minutes, and at 60° C. overnight. The reaction mixture was poured into ice water and extracted with ethyl acetate. The organic layer was washed with a saturated aqueous solution of sodium hydrogen carbonate, dried over magnesium sulfate, filtered through a silica gel layer and concentrated. The obtained solid residue was pulverized in hexane and diisopropyl ether, filtered, washed with hexane and dried to give the title compound (27.8 g, yield 77%).

¹H NMR (300 MHz, CDCl₃) δ ppm: 1.48 (t, J=7.0 Hz, 3 H), 4.20 (q, J=7.0 Hz, 2 H),.4.56 (s, 2 H), 7.06 (d, J=8.7 Hz, 1 H), 7.54 (dd, J=8.7, 2.3 Hz, 1 H), 7.86 (d, J=2.5 Hz, 1 H).

### Reference Example 9

### (4-ethoxy-3-nitrobenzyl)dimethylamine

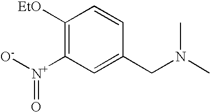

A solution of 4-ethoxy-3-nitrobenzyl chloride (27.8 g, 0.13 mol) in THF (70 mL) was added to 50% dimethylamine solution (130 mL) and the mixture was stirred at room temperature for 2 hours. The reaction solution was concentrated and extracted with ethyl acetate and a saturated aqueous solution of sodium hydrogen carbonate. The organic layer was dried over magnesium sulfate, filtered through aminosilica gel layer and concentrated to give the title compound (28.9 g).

¹H NMR (300 MHz, CDCl₃) δ ppm: 1.47 (t, J=7.0 Hz, 3 H), 2.23 (s, 6 H), 3.38 (s, 2 H), 4.18 (q, J=7.0 Hz, 2 H), 7.02 (d, J=8.7 Hz, 1 H), 7.46 (dd, J=8.6, 2.2 Hz, 1 H), 7.76 (d, J=2.1 Hz, 1 H).

### Reference Example 10

### (4-ethoxy-3-aminobenzyl)dimethylamine

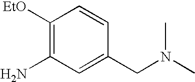

(4-Ethoxy-3-nitrobenzyl)dimethylamine (28.9 g, 0.13 mol) was dissolved in ethanol (130 mL) and 10% palladium-carbon (2.89 g) was added. Hydrazine monohydrate (19 mL) was added dropwise to the obtained suspension at room temperature over 40 minutes, and the mixture was further stirred for 20 minutes. The catalyst was filtered off and the mother liquor was concentrated. The residue was dissolved in ethyl acetate. The residual water was dried and removed from the obtained solution using magnesium sulfate, filtered through aminosilica gel layer and concentrated to give the title compound (26.5 g, containing ethyl acetate).

¹H NMR (300 MHz, CDCl₃) δ ppm: 1.42 (t, J=7.0 Hz, 3 H), 2.21 (s, 6 H), 3.28 (s, 2 H), 3.77 (s, 2 H), 4.05 (q, J=7.0 Hz, 2 H), 6.60 (dd, J=8.1, 1.9 Hz, 1 H), 6.71 (d, J=8.1 Hz, 1 H) 6.70 (d, J=1.9 Hz, 1 H).

The compounds described in the following Reference Examples 11-23 were produced in the similar manner as in Reference Example 1.

### Reference Example 11

### methyl (2R,3S)-3-(1H-indol-3-yl)-2-{[(4-phenylpiperazin-1-yl)carbonyl]amino}butanoate

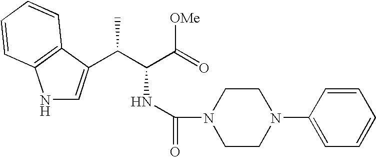

LC/MS (ESI) m/z 421 (M+H⁺).

### Reference Example 12

### methyl (2R,3S)-2-{[(4-benzylpiperidin-1-yl)carbonyl]amino}-3-(1H-indol-3-yl)butanoate

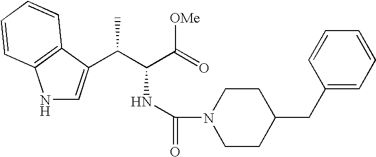

LC/MS (ESI) m/z 434 (M+H⁺).

### Reference Example 13

### methyl (2R,3S)-2-{[(4-benzylpiperazin-1-yl)carbonyl]amino}-3-(1H-indol-3-yl)butanoate

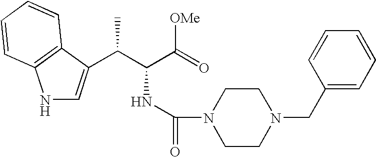

LC/MS (ESI) m/z 435 (M+H⁺).

### Reference Example 14

### methyl (2R,3S)-3-(1H-indol-3-yl)-2-[({4-[2-(trifluoromethyl)phenyl]piperidin-1-yl}carbonyl)amino]butanoate

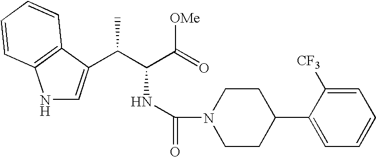

¹H NMR (400 MHz, CDCl₃) δ ppm: 1.51 (d, J=7.3 Hz, 3 H), 1.50-1.65 (m, 4 H), 2.77-2.87 (m, 2 H), 3.05 (t, J=11.6 Hz, 1 H), 3.64 (s, 3 H), 3.60-3.72 (m, 1 H), 3.93-4.05 (m, 2 H), 4.85 (dd, J=8.3, 5.4 Hz, 1 H), 5.01 (d, J=8.3 Hz, 1 H), 7.05 (d, J=2.2 Hz, 1 H), 7.12 (dt, J=7.2, 1.0 Hz, 1 H), 7.19 (dt, J=7.2, 1.0 Hz, 1 H), 7.28-7.37 (m, 3 H), 7.48-7.53 (m, 1 H), 7.62 (d, J=7.3 Hz, 1 H), 7.65 (d, J=7.8 Hz, 1 H), 8.12 (br, 1 H).

LC/MS (ESI) m/z 488 (M+H⁺).

### Reference Example 15

### methyl (2R,3S)-3-(1H-indol-3-yl)-2-(([4-(2-methoxyphenyl)piperidin-1-yl]carbonyl}amino)butanoate

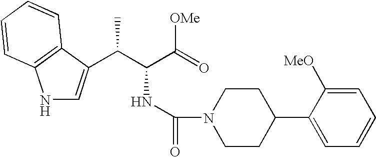

¹H NMR (400 MHz, CDCl₃) δ ppm: 1.51 (d, J=7.3 Hz, 3 H), 1.50-1.65 (m, 2 H), 1.75-1.77 (m, 2 H), 2.82 (td, J=7.0, 2.6 Hz, 1 H), 2.02 (td, J=7.0, 2.6 Hz, 1 H), 3.08 (tt, J=12.1, 3.4 Hz, 1 H), 3.62 (s, 3 H), 3.63-3.68 (m, 1 H), 3.82 (s, 3 H), 3.90-4.06 (m, 2 H), 4.84 (dd, J=8.6, 5.4 Hz, 1 H), 5.01 (d, J=8.3 Hz, 1 H), 6.85-7.22 (m, 7 H), 7.35 (d, J=8.1 Hz, 1 H), 7.64 (d, J=8.1 Hz, 1 H), 8.13 (brs, 1 H).

LC/MS (ESI) m/z 450 (M+H⁺).

### Reference Example 16

### methyl (2R,3S)-2-({[4-(2-fluorophenyl)piperidin-1-yl]carbonyl}amino)-3-(1H-indol-3-yl)butanoate

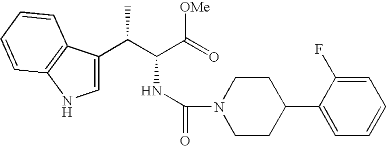

¹H NMR (400 MHz, CDCl₃) δ ppm: 1.51 (d, J=7.3 Hz, 3 H), 1.50-1.65 (m, 2 H), 1.75-1.77 (m, 2 H), 2.82 (td, J=7.0, 2.6 Hz, 1 H), 2.86 (td, J 7.0, 2.6 Hz, 1 H), 2.99 (tt, J=12.1, 3.4 Hz, 1 H), 3.62 (s, 3 H), 3.57-3.68 (m, 1 H), 3.90-4.06 (m, 2 H), 4.84 (dd, J=8.6, 5.4 Hz, 1 H), 5.01 (d, J=8.3 Hz, 1 H), 6.85-7.22 (m, 7 H), 7.35 (d, J=8.1 Hz, 1 H), 7.64 (d, J=8.1 Hz, 1 H), 8.13 (brs, 1 H).

LC/MS (ESI) m/z 438 (M+H⁺).

### Reference Example 17

### methyl (2R,3S)-2-({[4-(4-fluorophenyl)-3,6-dihydropyridin-1(2H)-yl]carbonyl}amino)-3-(1H-indol-3-yl)butanoate

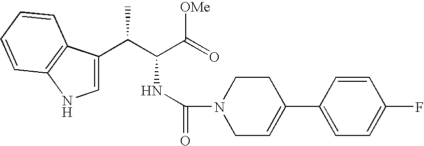

¹H NMR (400 MHz, CDCl₃) δ ppm: 1.51 (d, J=7.3 Hz, 3 H), 2.44-2.49 (m, 2 H), 3.49-3.71 (m, 3 H), 3.63 (s, 3 H), 3.83-3.97 (m, 2 H), 4.86 (dd, J=8.3, 5.4 Hz, 1 H), 4.99 (d, J=8.3 Hz, 1 H), 5.90 (s, 1 H), 7.00-7.20 (m, 5 H), 7.29-7.37 (m, 3 H), 7.62 (d, J=8.1 Hz, 1 H), 8.14 (s, 1 H).

LC/MS (ESI) m/z 436 (M+H⁺).

### Reference Example 18

### methyl (2R,3S)-3-(1H-indol-3-yl)-2-(([4-[2-(trifluoromethyl)phenyl]-3,6-dihydropyridin-1(2H)-yl]carbonyl}amino)butanoate

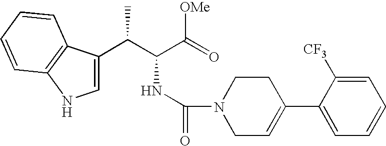

¹H NMR (400 MHz, CDCl₃) δ ppm: 1.51 (d, J=7.1 Hz, 3 H), 2.31-2.40 (m, 2 H), 3.48-3.72 (m, 3 H), 3.63 (s, 3 H), 3.81-3.94 (m, 2 H), 4.88 (dd, J=7.3, 5.4 Hz, 1 H), 5.00 (d, J=8.3 Hz, 1 H), 5.52 (s, 1 H), 7.04-7.22 (m, 4 H), 7.34-7.39 (m, 2 H), 7.47-7.51 (m, 1 H), 7.61-7.66 (m, 2 H), 8.21 (brs, 1 H).

LC/MS (ESI) m/z 486 (M+H⁺).

### Reference Example 19

### methyl (2R,3S)-2-{[(4-hydroxy-4-phenylpiperidin-1-yl)carbonyl]amino}-3-(1H-indol-3-yl)butanoate

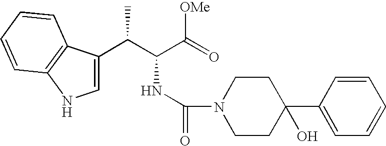

¹H NMR (400 MHz, CDCl₃) δ ppm: 1.50 (d, J=7.3 Hz, 3 H), 1.67-1.71 (m, 3 H), 1.93 (dt, J=13.5, 4.8 Hz, 2 H), 3.19-3.30 (m, 2 H), 3.62 (s, 3 H), 3.57-3.81 (m, 3 H), 4.84 (dd, J=8.3, 5.4 Hz, 1 H), 5.03 (d, J=8.3 Hz, 1 H), 7.03 (d, J=2.4 Hz, 1 H), 7.09 (td, J=7.1, 1.0 Hz, 1 H), 7.17 (td, J=7.1, 1.0 Hz, 1 H), 7.26-7.45 (m, 6 H), 7.61 (d, J=8.1 Hz, 1 H), 8.13 (s, 1 H).

LC/MS (ESI) m/z 436 (M+H⁺)

### Reference Example 20

### methyl (2R,3S)-2-({[4-(4-chlorophenyl)-4-hydroxypiperidin-1-yl]carbonyl}amino)-3-(1H-indol-3-yl)butanoate

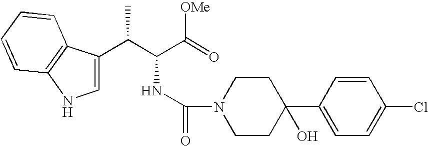

¹H NMR (400 MHz, CDCl₃) δ ppm: 1.50 (d, J=7.3 Hz, 3 H), 1.61-1.68 (m, 2 H), 1.86 (dt, J=12.9, 4.3 Hz, 1 H), 1.93 (dt, J=12.9, 4.3 Hz, 1 H), 3.20 (dt, J=13.2, 2.5 Hz, 1 H), 3.23 (dt, J=13.2, 2.5 Hz, 1 H), 3.63 (s, 3 H), 3.58-3.79 (m, 3 H), 4.83 (dd, J=8.4, 5.4 Hz, 1 H), 5.02 (d, J=8.3 Hz, 1 H), 7.03 (d, J=2.4 Hz, 1 H), 7.09 (dt, J=7.1, 1.2 Hz, 1 H), 7.17 (dt, J=7.1, 1.2 Hz, 1 H), 7.31-7.37 (m, 5 H), 7.61 (d, J=8.1 Hz, 1 H), 8.17 (s, 1 H).

LC/MS (ESI) m/z 470 (M+H⁺).

### Reference Example 21

### methyl (2R,3S)-2-({[4-hydroxy-4-(2-methylphenyl)piperidin-1-yl]carbonyl}amino)-3-(1H-indol-3-yl)butanoate

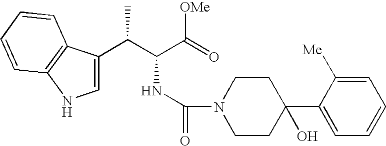

¹H NMR (400 MHz, CDCl₃) δ ppm:1.51 (d, J=7.3 Hz, 3 H), 1.89-2.10 (m, 4 H), 2.60 (s, 3 H), 3.26-3.36 (m, 2 H), 3.58-3.83 (m, 3 H), 3.62 (s, 3 H), 4.84 (dd, J=8.3, 5.4 Hz, 1 H), 5.04 (d, J=8.3 Hz, 1 H), 7.04 (d, J=2.4 Hz, 1 H), 7.08-7.37 (m, 7 H), 7.63 (d, J=7.8 Hz, 1 H), 8.10 (s, 1 H).

LC/MS (ESI) m/z 450 (M+H⁺).

### Reference Example 22

### methyl (2R,3S)-3-(1H-indol-3-yl)-2-({[4-(1-naphthyl)piperidin-1-yl]carbonyl}amino)butanoate

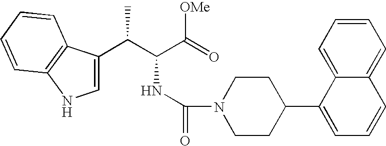

¹H NMR (400 MHz, CDCl₃) δ ppm: 1.52 (d, J=7.5 Hz, 3 H), 1.65-1.97 (m, 4 H), 2.95 (td, J=12.6, 3.1 Hz, 1 H), 3.00 (td, J=12.7, 2.4 Hz, 1 H), 3.46 (tt, J=12.0, 3.3 Hz, 1 H), 3.58-3.73 (m, 2 H), 3.64 (s, 3 H), 4.01 (d, J=13.4 Hz, 1 H), 4.86 (dd, J=8.3, 5.1 Hz, 1 H), 5.05 (d, J=8.3 Hz, 1 H), 7.05 (d, J=2.4 Hz, 1 H), 7.12 (td, J=7.9, 1.0 Hz, 1 H), 7.18 (td, J=7.9, 1.0 Hz, 1 H), 7.34 (dd, J=14.4, 7.6 Hz, 2 H), 7.43-7.54 (m, 3 H), 7.65 (d, J=7.6 Hz, 1 H), 7.73 (d, J 8.3 Hz, 1 H), 7.88 (d, J=7.6 Hz, 1 H), 8.07 (d, J=8.6 Hz, 1 H), 8.13 (brs, 1 H).

LC/MS (ESI) m/z 470 (M+H⁺).

### Reference Example 23

### methyl (2R,3S)-2-{[(4-benzoylpiperazin-1-yl)carbonyl]amino)-3-(1H-indol-3-yl)butanoate

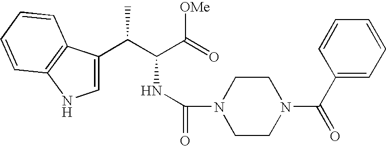

LC/MS (ESI) m/z 449 (M+H⁺).

### Reference Example 24

### ethyl (2R,3S)-3-(1H-indol-3-yl)-2-([(4-phenylpiperidin-1-yl)carbonyl]amino}butanoate

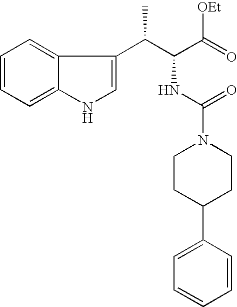

A mixture of ethyl (2R,3S)-2-amino-3-(1H-indol-3-yl)butanoate O,O′-diacetyl-L-tartarate (7.21 g) and a saturated aqueous solution of sodium hydrogen carbonate (35 mL)—ethyl acetate (40 mL) was stirred at room temperature for 2 hrs. The organic layer was separated and the aqueous layer was extracted with ethyl acetate. The obtained organic layer was dried (MgSO₄) and the solvent was evaporated to give ethyl (2R,3S)-2-amino-3-(1H-indol-3-yl)butanoate as a colorless oil (3.69 g, yield 100%).

To a solution of the obtained oil (2.22 g) and N,N-diisopropylethylamine (1.98 mL) in acetonitrile (60 mL) was added N,N′-disuccinimidyl carbonate (2.65 g) under ice-cooling, and the mixture was stirred for 1 hr. To the obtained solution was added a solution of 4-phenylpiperidine hydrochloride (2.25 g) and DBU (1.71 mL) in acetonitrile (10 mL) and N,N-diisopropylethylamine (1.98 mL) was added under ice-cooling. The reaction solution was stirred at room-temperature for 16 hrs. and a saturated solution of sodium hydrogen carbonate was added. The mixture was extracted with ethyl acetate and the extract was purified by silica gel column chromatography (developing solvent: hexane/ethyl acetate=1/1-1/4) to give the title compound as a white amorphous powder (3.62 g, yield 93%).

LC/MS (ESI) m/z 434 (M+H⁺).

The compounds described in the following Reference Examples 25-54 were produced in the similar mariner as in Reference Example 24.

### Reference Example 25

### ethyl (2R,3S)-3-(1H-indol-3-yl)-2-({[4-(2-methylphenyl)piperidin-1-yl]carbonyl}amino)butanoate

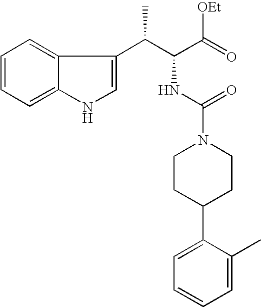

¹H NMR (400 MHz, CDCl₃) δ ppm: 1.11 (t, J=7.1 Hz, 3 H), 1.51 (d, J=7.3 Hz, 3 H), 1.56-1.73 (m, 4 H), 2.33 (s, 3 H), 2.77-2.89 (m, 3 H), 3.63-3.74 (m, 1 H), 3.93-4.10 (m, 2 H), 4.06 (q, J=7.1 Hz, 2 H), 4.83 (dd, J=8.6, 5.6 Hz, 1 H), 5.05 (d, J=15.8 Hz, 1 H), 7.03-7.23 (m, 6 H), 7.26-7.38 (m, 2 H), 7.65 (d, J=7.8 Hz, 1 H), 8.07 (brs, 1 H).

LC/MS (ESI) m/z 448 (M+H⁺).

### Reference Example 26

### ethyl (2R,3S)-2-({[4-(2-ethylphenyl)piperidin-1-yl]carbonyl}amino)-3-(1H-indol-3-yl)butanoate

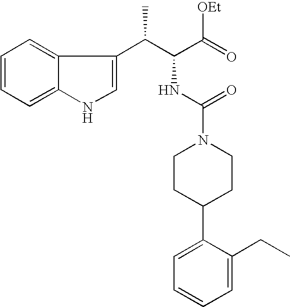

¹H NMR (400 MHz, CDCl₃) δ ppm: 1.12 (t, J 7.1 Hz, 3 H), 1.21 (t, J=7.6 Hz, 3 H), 1.52 (d, J=7.3 Hz, 3 H), 1.58-1.72 (m, 4 H), 2.68 (q, J=7.6 Hz, 2 H), 2.77 (m, 3 H), 3.64-3.67 (m, 1 H), 3.94-4.15 (m, 2 H), 4.06 (q, J=7.1 Hz, 2 H), 4.82 (dd, J=8.4, 5.7 Hz, 1 H), 5.07 (d, J=8.6 Hz, 1 H), 7.05 (d, J=2.4 Hz, 1 H), 7.09-7.21 (m, 6 H), 7.36 (d, J=8.1 Hz, 1 H), 7.65 (d, J=8.1 Hz, 1 H), 8.43 (brs, 1 H).

LC/MS (ESI) m/z 462 (M+H⁺).

### Reference Example 27

### ethyl (2R,3S)-3-(1H-indol-3-yl)-2-({[4-(3-methylphenyl)piperidin-1-yl]carbonyl}amino)butanoate

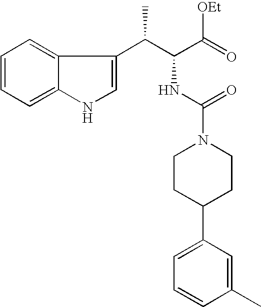

¹H NMR (400 MHz, CDCl₃) δ ppm: 1.14 (t, J=7.1 Hz, 3 H), 1.51 (d, J=7.3 Hz, 3 H), 1.55-1.62 (m, 2 H), 1.77-1.80 (m, 2 H), 3.40 (s, 3 H), 2.59 (tt, J=12.2, 3.5 Hz, 1 H), 2.79-2.87 (m, 2 H), 3.63-3.66 (m, 1 H), 3.91-4.09 (m, 2 H), 4.07 (q. J=7.1Hz, 2 H), 4.83 (dd, J=8.5, 5.6 Hz, 1 H), 5.02 (d, J=8.5 Hz, 1 H), 6.96-7.21 (m, 7 H), 7.35 (d, J=8.1 Hz, 1 H), 7.64 (d, J=8.1 Hz, 1 H), 8.09 (brs, 1 H).

LC/MS (ESI) m/z 448 (M+H⁺).

### Reference Example 28

### ethyl (2R,3S)-3-(1H-indol-3-yl)-2-[((4-[3-(trifluoromethyl)phenyl]piperidin-1-yl}carbonyl)amino]butanoate

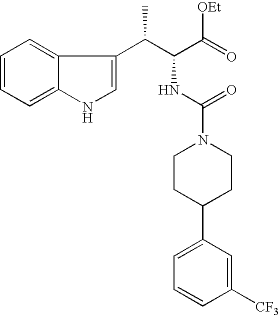

¹H NMR (400 MHz, CDCl₃) δ ppm: 1.31 (t, J=7.1 Hz, 3 H), 1.51 (d, J=7.3 Hz, 3 H), 1.54-1.69 (m, 2 H), 1.77-1.82 (m, 2 H), 2.70 (tt, J=12.0, 2.6 Hz, 1 H), 2.80 (td, J=13.0, 2.6 Hz, 1 H), 2.85 (td, J=13.0, 2.6 Hz, 1 H), 3.64-3.69 (m, 1 H), 3.94-4.03 (m, 1 H), 4.07 (q. J=7.1 Hz, 2 H), 4.83 (dd, J=8.4, 5.5 Hz, 1 H), 5.02 (d, J=8.3 Hz, 1 H), 7.04 (d, J=2.4 Hz, 2 H), 7.08-7.52 (m, 8 H), 7.64 (d, J=7.0 Hz, 1 H), 8.08 (brs, 1 H).

LC/MS (ESI) m/z 502 (M+H⁺).

### Reference Example 29

### ethyl (2R,3S)-3-(1H-indol-3-yl)-2-({[4-(3-methoxyphenyl)piperidin-1-yl]carbonyl}amino)butanoate

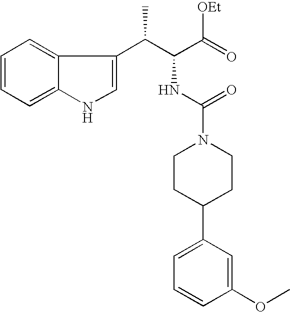

¹H NMR (400 MHz, CDCl₃) δ ppm: 1.12 (t, J=7.1 Hz, 3 H), 1.51 (d, J=7.3 Hz, 3 H), 1.55-1.64 (m, 2 H) 1.78-1.81 (m, 2 H), 2.61 (tt, J=12.1, 3.5 Hz, 1 H), 2.75 (td, J=12.8, 2.5 Hz, 1 H), 2.84 (td, J=12.8, 2.5 Hz, 1 H), 3.61-3.69 (m, 1 H), 3.80 (s, 3 H), 3.91-3.98 (m, 2 H), 4.06 (q, J=7.1 Hz, 2 H), 4.83 (dd, J=8.3, 5.6 Hz, 1 H), 5.03 (d, J=8.6 Hz, 1 H), 6.73-6.79 (m, 3 H), 7.04 (d, J=2.5 Hz, 1 H), 7.10 (td, J=7.1, 1.0 Hz, 1 H), 7.12 (td, J=7.1, 1.0 Hz, 1 H), 7.23 (t, J=7.8 Hz, 1 H), 7.35 (d, J=8.1 Hz, 1 H), 7.64 (d, J=7.8 Hz, 1 H), 8.16 (s, 1 H).

LC/MS (ESI) m/z 464 (M+H⁺).

### Reference Example 30

### ethyl (2R,3S)-2-({[4-(4-chlorophenyl)-3,6-dihydropyridin-1(2H)-yl]carbonyl}amino)-3-(1H-indol-3-yl)butanoate

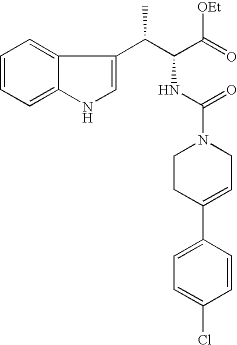

¹H NMR (400 MHz, CDCl₃) δ ppm: 1.12 (t, J=7.1 Hz, 3 H), 1.53 (d, J=7.3 Hz, 3 H), 2.40-2.51 (m, 2 H), 3.50-3.68 (m, 3 H), 3.85-3.94 (m, 2 H), 4.06 (q, J=7.1 Hz, 2 H), 4.84 (dd, J=8.5, 5.5 Hz, 1 H), 5.00 (d, J=8.3 Hz, 1 H), 5.94-5.97 (m, 1 H), 7.03-7.19 (m, 3 H), 7.26-7.37 (m, 5 H), 7.62 (d, J=7.3 Hz, 1 H), 8.09 (brs, 1 H).

LC/MS (ESI) m/z 466 (M+H⁺).

### Reference Example 31

### ethyl (2R,3S)-2-({[4-hydroxy-4-(1,3-thiazol-2-yl)piperidin-1-yl]carbonyl}amino)-3-(1H-indol-3-yl)butanoate

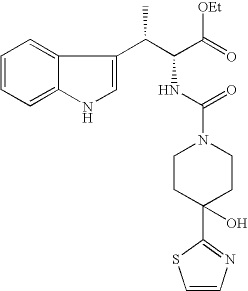

¹H NMR (400 MHz, CDCl₃) δ ppm: 1.13 (t, J=7.1 Hz, 3 H), 1.50 (d, J=7.3 Hz, 3 H), 1.80-1.85 (m, 2 H), 2.03,-2.12 (m, 3 H), 3.22-3.35 (m, 2 H), 3.62-3.83 (m, 3 H), 4.07 (q, J=7.1 Hz, 2 H), 4.82 (dd, J=8.5, 5.5 Hz, 1 H), 5.04 (d, J=8.5 Hz, 1 H), 7.03 (d, J=2.4 Hz, 1 H), 7.08-7.19 (m, 2 H), 7.31 (d, J=3.2 Hz, 1 H), 7.34 (d, J=8.1 Hz, 1 H), 7.63 (d, J=7.5 Hz, 1 H), 7.73 (d, J=3.2 Hz, 1 H), 8.14 (s, 1 H).

LC/MS (ESI) m/z 457 (M+H⁺).

### Reference Example 32

### ethyl (2R,3S)-3-(1H-indol-3-yl)-2-({[4-(4-methyl-1,3-thiazol-2-yl)piperidin-1-yl]carbonyl}amino)butanoate

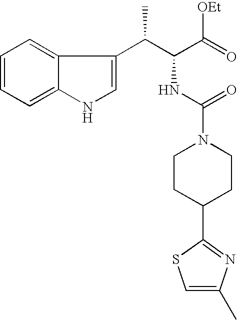

¹H NMR (400 MHz, CDCl₃) δ ppm: 1.12 (t, J=7.1 Hz, 3 H), 1.50 (d, J=7.3 Hz, 3 H), 1.63-1.76 (m, 2 H), 2.04-2.07 (m, 2 H), 2.42 (s, 3 H), 2.79-2.93 (m, 2 H), 3.11 (tt, J=11.6, 3.8 Hz, 1 H), 3.60-3.68 (m, 1 H), 3.95 (dd, J=38.9, 13.3 Hz, 2 H), 4.08 (q, J=7.1 Hz, 2 H), 4.81 (dd, J=8.4, 5.5 Hz, 1 H), 5.01 (d, J=8.4 Hz, 1 H), 6.76 (d, J=1.0 Hz, 1 H), 7.04 (d, J=2.4 Hz, 1 H), 7.10 (dt, J=8.1, 1.1 Hz, 1 H), 7.17 (dt, J=8.1, 1.1 Hz, 1 H), 7.35 (d, J=8.1 Hz, 1H), 7.62 (d, J=7.6 Hz, 1 H), 8.12 (s, 1 H).

LC/MS (ESI) m/z 455 (M+H⁺).

### Reference Example 33

### ethyl (2R,3S)-2-({[4-(4-fluorophenoxy)piperidin-1-yl]carbonyl}amino)-3-(1H-indol-3-yl)butanoate

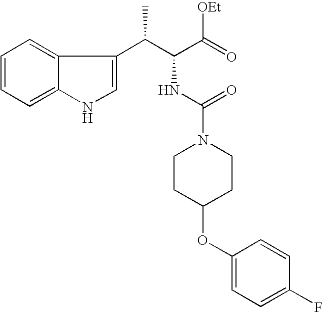

LC/MS (ESI) m/z 468 (M+H⁺).

### Reference Example 34

### ethyl (2R,3S)-2-({[4-(4-fluorophenyl)piperidin-1-yl]carbonyl}amino)-3-(1H-indol-3-yl)butanoate

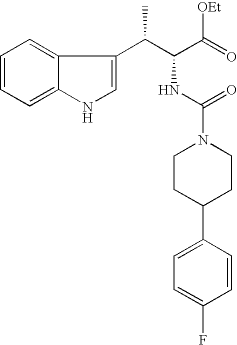

LC/MS (ESI) m/z 452 (M+H⁺).

### Reference Example 35

### ethyl (2R,3S)-2-({[4-(4-chlorophenyl)piperidin-1-yl]carbonyl}amino)-3-(1H-indol-3-yl)butanoate

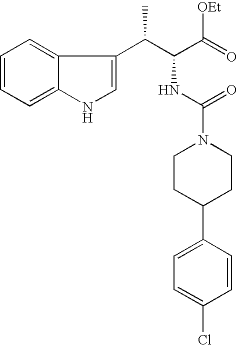

LC/MS (ESI) m/z 468 (M+H⁺)

### Reference Example 36

### ethyl (2R,3S)-3-(1H-indol-3-yl)-2-({[4-(4-methylphenyl)piperidin-1-yl]carbonyl}amino)butanoate

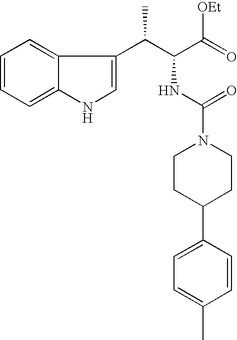

LC/MS (ESI) m/z 448 (M+H⁺).

### Reference Example 37

### ethyl (2R,3S)-3-(1H-indol-3-yl)-2-({[4-(4-methoxyphenyl)piperidin-1-yl]carbonyl)amino)butanoate

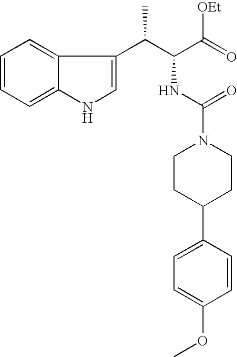

LC/MS (ESI) m/z 464 (M+H⁺).

### Reference Example 38

### ethyl (2R,3S)-2-({[4-(2,4-difluorophenyl)piperidin-1-yl]carbonyl}amino)-3-(1H-indol-3-yl)butanoate

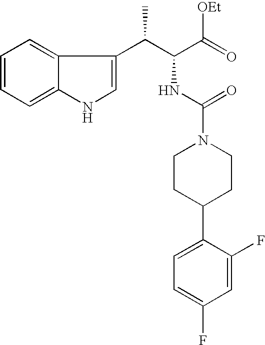

LC/MS (ESI) m/z 470 (M+H⁺).

### Reference Example 39

### ethyl (2R,3S)-2-({[4-(3-fluorophenyl)piperidin-1-yl]carbonyl}amino)-3-(1H-indol-3-yl)butanoate

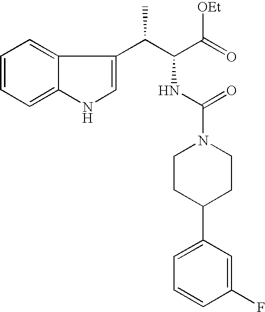

LC/MS (ESI) m/z 452 (M+H⁺).

### Reference Example 40

### ethyl (2R,3S)-3-(1H-indol-3-yl)-2-({[4-(2-methylphenyl)piperazin-1-yl]carbonyl}amino)butanoate

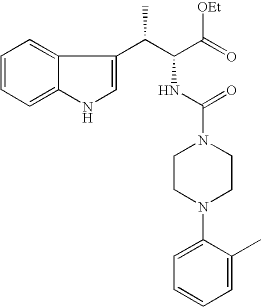

LC/MS (ESI) m/z 449 (M+H⁺).

### Reference Example 41

### ethyl (2R,3S)-2-({[4-(4-fluorophenyl)piperazin-1-yl]carbonyl}amino)-3-(1H-indol-3-yl)butanoate

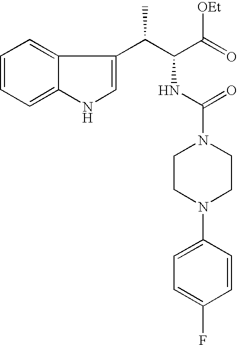

LC/MS (ESI) m/z 453 (M+H⁺).

### Reference Example 42

### ethyl (2R,3S)-2-{[(4-cyclohexylpiperazin-1-yl)carbonyl]amino)-3-(1H-indol-3-yl)butanoate

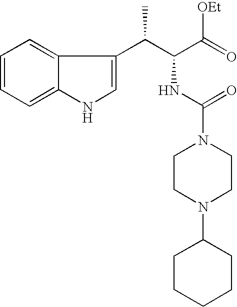

LC/MS (ESI) m/z 441 (M+H⁺).

### Reference Example 43

### ethyl (2R,3S)-2-(([4-(4-fluorobenzoyl)piperidin-1-yl]carbonyl}amino)-3-(1H-indol-3-yl)butanoate

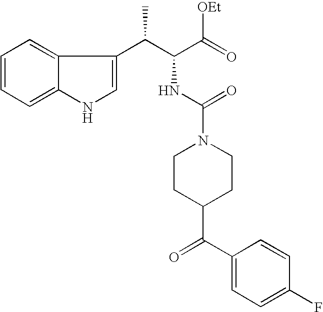

LC/MS (ESI) m/z 480 (M+H⁺).

### Reference Example 44

### ethyl (2R,3S)-2-[({4-[(4-fluorophenyl)thiolpiperidin-1-yl}carbonyl)amino]-3-(1H-indol-3-yl)butanoate

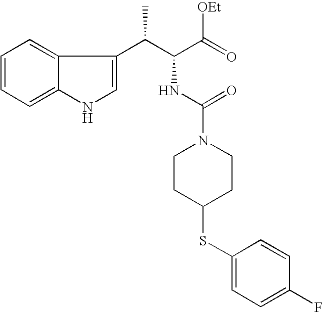

LC/MS (ESI) m/z 484 (M+H⁺).

### Reference Example 45

### ethyl (2R,3S)-2-[({4-[(4-fluorophenyl)sulfonyl]piperidin-1-yl}carbonyl)amino]-3-(1H-indol-3-yl)butanoate

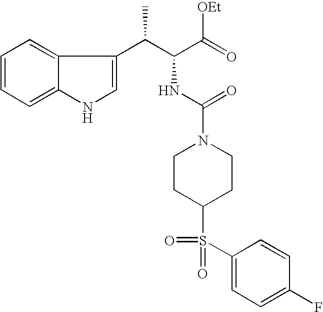

LC/MS (ESI) m/z 516 (M+H⁺).

### Reference Example 46

### ethyl (2R,3S)-2-[({4-[(4-fluorophenyl)sulfonyl]piperazin-1-yl}carbonyl)amino]-3-(1H-indol-3-yl)butanoate

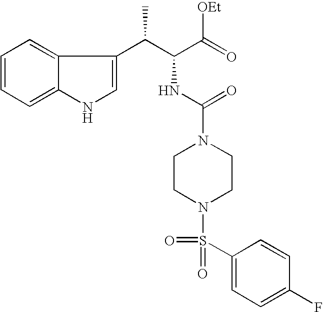

LC/MS (ESI) m/z 517 (M+H⁺).

### Reference Example 47

### ethyl (2R,3S)-2-[(3,4-dihydroisoquinolin-2(1H)-ylcarbonyl)amino]-3-(1H-indol-3-yl)butanoate

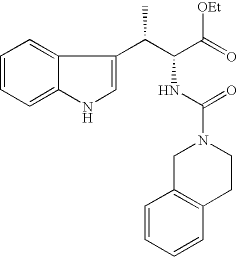

LC/MS (ESI) m/z 406 (M+H⁺).

### Reference Example 48

### ethyl (2R,3S)-3-(1H-indol-3-yl)-2-{[(6-methyl-3,4-dihydroisoquinolin-2(1H)-yl)carbonyl]amino)butanoate

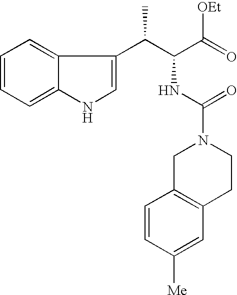

LC/MS (ESI) m/z 420 (M+H⁺).

### Reference Example 49

### ethyl (2R,3S)-2-{[(6-chloro-3,4-dihydroisoquinolin-2(1H)-yl)carbonyl]amino}-3-(1H-indol-3-yl)butanoate

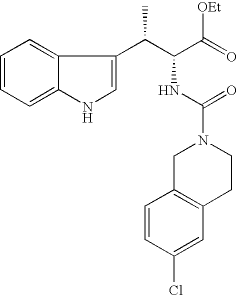

LC/MS (ESI) m/z 440 (M+H⁺).

### Reference Example 50

### ethyl (2R,3S)-2-{[(6-fluoro-3,4-dihydroisoquinolin-2(1H)-yl)carbonyl]amino}-3-(1H-indol-3-yl)butanoate

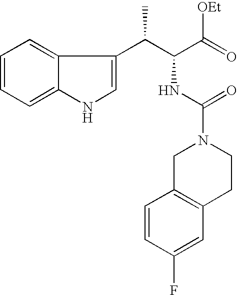

LC/MS (ESI) m/z 424 (M+H⁺).

### Reference Example 51

### ethyl (2R,3S)-2-[(4,7-dihydrothieno[2,3-c]pyridin-6(5H)-ylcarbonyl)amino]-3-(1H-indol-3-yl)butanoate

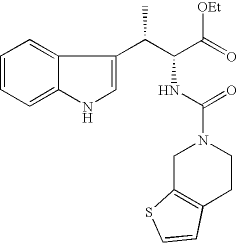

LC/MS (ESI) m/z 412 (M+H⁺).

### Reference Example 52

### ethyl (2R,3S)-3-(1H-indol-3-yl)-2-[(1,2,4,5-tetrahydro-3H-3-benzazepin-3-ylcarbonyl)amino]butanoate

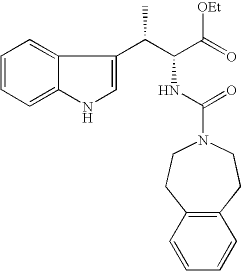

LC/MS (ESI) m/z 420 (M+H⁺).

### Reference Example 53

### ethyl (2R,3S)-2-[(1,3-dihydro-2H-isoindol-2-ylcarbonyl)amino]-3-(1H-indol-3-yl)butanoate

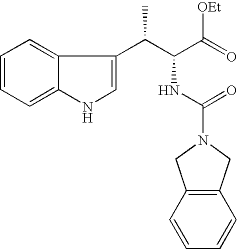

LC/MS (ESI) m/z 392 (M+H⁺).

### Reference Example 54

### ethyl (2R,3S)-2-[(2,3-dihydro-1H-indol-1-ylcarbonyl)amino]-3-(1H-indol-3-yl) butanoate

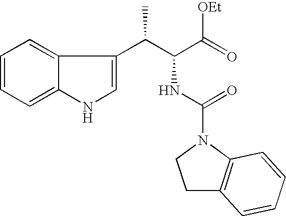

LC/MS (ESI) m/z 392 (M+H⁺).

### Reference Example 55

### methyl (2RS,3SR)-2-({[4-(4-fluorophenoxy)piperidin-1-yl]carbonyl}amino)-3-(1H-indol-3-yl)butanoate

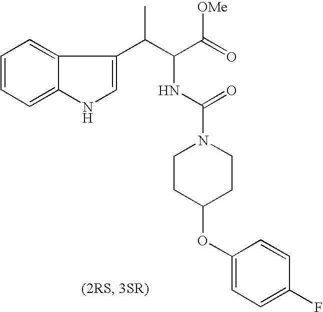

To a solution of methyl (2RS,3SR)-2-amino-3-(1H-indol-3-yl)butanoate (4.79 g) and N,N-diisopropylethylamine (4.30 mL) in acetonitrile (70 mL) was added N,N′-disuccinimidyl carbonate (5.53 g) under ice-cooling and the mixture was stirred for 1 hr. To the obtained solution were added a solution of 4-(4-fluorophenyl)piperidine hydrochloride (5.00 g) and DBU (3.23 mL) in acetonitrile (10 mL) and N,N-diisopropylethylamine (4.30 mL) under ice-cooling. The reaction solution was stirred at room temperature for 12 hrs. and a saturated solution of sodium hydrogen carbonate was added. The mixture was extracted with ethyl acetate. The extract was purified by silica gel column chromatography (developing solvent; ethyl acetate) to give the title compound as a pale yellow oil (8.70 g, yield 93%).

¹H NMR (300 MHz, CDCl₃) δ ppm: 1.49 (d, J=7.5 Hz, 3 H), 1.60-1.73 (m, 2 H), 1.80-1.90 (m, 2 H), 3.14-3.29 (m, 2 H), 3.45-3.62 (m, 6 H), 4.31-4.37 (m, 1 H), 4.81 (dd, J=5.4, 8.1 Hz, 1 H), 4.98 (d, J=8.4 Hz, 1 H), 6.80-6.86 (m, 2 H), 6.92-7.02 (m, 4 H), 7.07-7.20 (m, 2 H), 7.34 (d, J=8.1 Hz, 1 H), 7.60 (d, J=7.8 Hz, 1 H), 8.14 (s, 1 H).

### Reference Example 56

### methyl (2S,3R)-3-(1H-indol-3-yl)-2-{[(4-phenylpiperidin-1-yl)carbonyl]amino}butanoate

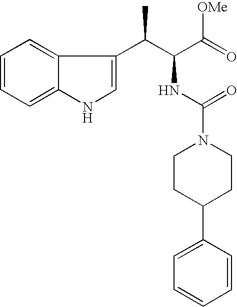

To a solution of methyl (2S,3R)-2-amino-3-(1H-indol-3-yl)butanoate (1.16 g) and N,N-diisopropylethylamine (1.05 mL) in acetonitrile (30 mL) was added N,N′-disuccinimidyl carbonate (1.39 g) under ice-cooling and the mixture was stirred for 1 hr. To the obtained solution were added a solution of 4-phenylpiperidine (0.967 g) in acetonitrile (5 mL) and N,N-diisopropylethylamine (1.05 mL) under ice-cooling. The reaction solution was stirred at room temperature for 16 hrs. and a saturated solution of sodium hydrogen carbonate was added. The mixture was extracted with ethyl acetate. The extract was dried (MgSO₄) and the solvent was evaporated. The residue was purified by silica gel column chromatography (developing solvent: hexane/ethyl acetate=1/1-1/4) to give the title compound as a white amorphous powder (2.07 g, yield 99%).

LC/MS (ESI) m/z 420 (M+H⁺).

The compound described in the following Reference Example 57 was produced in the similar manner as in Reference Example 56.

### Reference Example 57

### methyl (2S,3R)-2-({[4-(4-fluorophenyl)piperazin-1-yl]carbonyl}amino)-3-(1H-indol-3-yl)butanoate

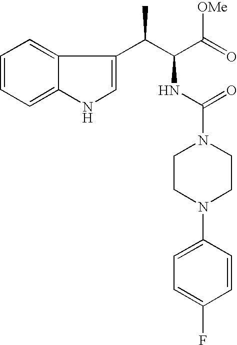

LC/MS (ESI) m/z 439 (M+H⁺).

### Reference Example 58

### methyl (2RS,3SR)-2-{[(1-benzoylpiperidin-4-yl)carbonyl]amino}-3-(1H-indol-3-yl)butanoate

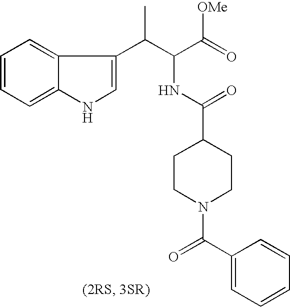

A mixed solution of methyl (2RS,3SR)-2-amino-3-(1H-indol-3-yl)butanoate (4.65 g), 1-benzoylpiperidine-4-carboxylic acid (5.13 g), WSC (5.75 g) and HOBt (4.60 g) in tetrahydrofuran (70 mL) was stirred at room temperature for 12 hrs. The reaction solution was diluted with ethyl acetate, a saturated aqueous sodium carbonate solution was added and the mixture was subjected to extraction. The organic layer was dried (MgSO₄) and the solvent was evaporated. The residue was purified by silica gel column chromatography (developing solvent: hexane/ethyl acetate=1/1−ethyl acetate) to give the title compound as a pale yellow oil (7.86 g, yield 88%).

¹H NMR (300 MHz, CDCl₃) δ ppm: 1.47 (d, J=6.9 Hz, 3 H), 1.63-1.83 (m, 4 H), 2.27-2.35 (m, 1 H), 2.80-3.00 (m, 2 H), 3.62-3.78 (m, 4 H), 4.60 (brs, 1 H), 4.86-4.96 (m, 1 H), 5.86-6.02 (m, 1 H), 6.99 (d, J=3.6 Hz, 1 H), 7.08-7.20 (m, 2 H), 7.33-7.40 (m, 6 H), 7.55-7.60 (m, 1 H), 8.22-8.28 (m, 1 H).

### Reference Example 59

### methyl (2R,3S)-2-{[(1-benzoylpiperidin-4-yl)carbonyl]amino}-3-(1H-indol-3-yl)butanoate

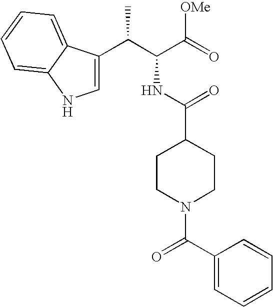

A mixed solution of methyl (2R,3S)-2-amino-3-(1H-indol-3-yl)butanoate (1.28 g), 1-benzoylpiperidine-4-carboxylic acid (1.54 g), WSC (1.58 g) and HOBt (1.10 g) in acetonitrile (50 mL) was stirred at room temperature for 16 hrs. The reaction solution was diluted with ethyl acetate, a saturated aqueous solution of sodium carbonate was added and the mixture was subjected to extraction. The extract was purified by silica gel column chromatography (developing solvent: hexane/ethyl acetate=1/1-1/4−ethyl acetate) to give the title compound as a pale yellow powder (2.01 g, yield 82%).

LC/MS (ESI) m/z 448 (M+H⁺).

### Reference Example 60

### (2RS,3SR)-2-({[4-(4-fluorophenoxy)piperidin-1-yl]carbonyl}amino)-3-(1H-indol-3-yl)butanoic acid

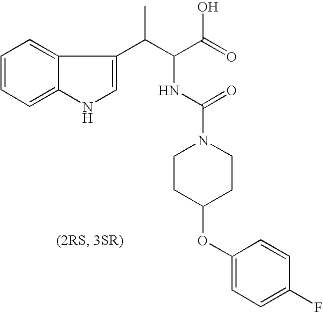

To a solution of methyl (2RS,3SR)-2-({[4-(4-fluorophenoxy)piperidin-1-yl]carbonyl}amino)-3-(1H-indol-3-yl)butanoate (8.70 g) in methanol (45 mL) was added 2N aqueous sodium hydroxide (22 mL) at room temperature and the mixture was stirred for 2 hrs. The reaction solution was neutralized with 1N hydrochloric acid (44 mL) and the mixture was extracted with ethyl acetate. The extract was dried (MgSO₄) and the solvent was evaporated under reduced pressure to give the title compound as a pale orange amorphous powder (8.00 g, yield 95%).

LC/MS (ESI) m/z 440 (M+H⁺).

The compounds described in the following Reference Examples 61-64 were produced in the similar manner as in Reference Example 60.

### Reference Example 61

### (2S,3R)-3-(1H-indol-3-yl)-2-{[(4-phenylpiperidin-1-yl)carbonyl]amino}butanoic acid

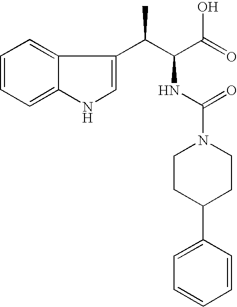

LC/MS (ESI) m/z 406 (M+H⁺).

### Reference Example 62

### (2S,3R)-2-({[4-(4-fluorophenyl)piperazin-1-yl]carbonyl}amino)-3-(1H-indol-3-yl)butanoic acid

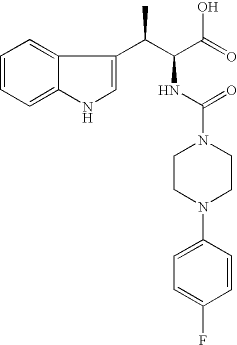

LC/MS (ESI) m/z 425 (M+H⁺).

### Reference Example 63

### (2RS,3SR)-2-{[(4-benzoylpiperidin-1-yl)carbonyl]amino}-3-(1H-indol-3-yl)butanoic acid

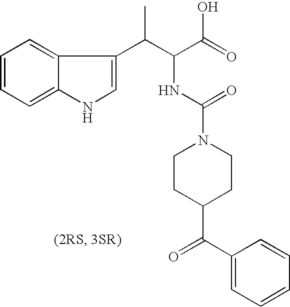

LC/MS (ESI) m/z 434 (M+H⁺).

### Reference Example 64

### (2R,3S)-2-{[(1-benzoylpiperidin-4-yl)carbonyl]amino}-3-(1H-indol-3-yl)butanoic acid

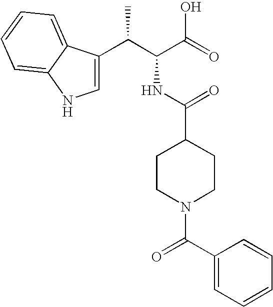

LC/MS (ESI) m/z 434 (M+H⁺).

### Reference Example 65

### methyl (2R)-2-({[4-(4-fluorophenoxy)piperidin-1-yl]carbonyl}amino)-3-(1H-indol-3-yl)propanoate

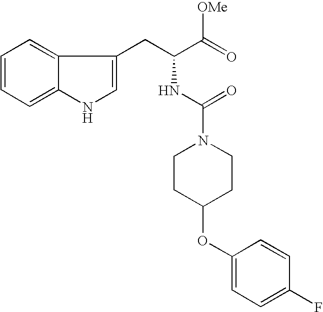

To a solution of methyl (2R)-2-amino-3-(1H-indol-3-yl)propanoate hydrochloride (345 mg), DBU (0.21 mL) and N,N-diisopropylethylamine (0.293 mL) in acetonitrile (10 mL) was added N,N′-disuccinimidyl carbonate (390 mg) under ice-cooling and the mixture was stirred for 1 hr. To the reaction solution lo were added a solution of 4-(4-fluorophenyl)piperidine hydrochloride (390 mg) and DBU (0.252 mL) in acetonitrile (1 mL) and N,N-diisopropylethylamine (0.293 mL) under ice-cooling. The reaction solution was stirred at room temperature for 16 hrs. and a saturated agueous solution of sodium hydrogen carbonate was added. The mixture was extracted with ethyl acetate. The organic layer was dried (MgSO₄) and the solvent was evaporated. The residue was purified by silica gel column chromatography (developing solvent: hexane/ethyl acetate=1/1-1/2) to give the title compound as a pale yellow amorphous powder (0.52 g, yield 85%).

LC/MS (ESI) m/z 440 (M+H⁺).

The compound described in the following Reference Example 66 was produced in the similar manner as in Reference Example 65.

### Reference Example 66

### methyl (2R)-3-(1H-indol-3-yl)-2-{[(4-phenylpiperidin-1-yl)carbonyl]amino}propanoate

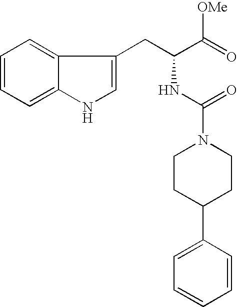

LC/MS (ESI) m/z 406 (M+H⁺).

The compounds described in the following Reference Examples 67-68 were produced in the similar manner as in Reference Example 2.

### Reference Example 67

### (2R)-2-({[4-(4-fluorophenoxy)piperidin-1-yl]carbonyl}amino)-3-(1H-indol-3-yl)propanoic acid

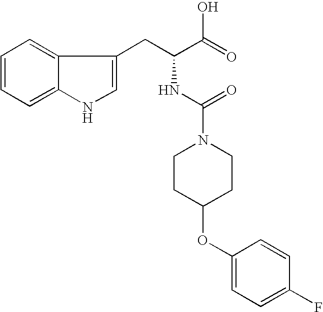

LC/MS (ESI) m/z 426 (M+H⁺).

### Reference Example 68

### (2R)-3-(1H-indol-3-yl)-2-{[(4-phenylpiperidin-1-yl)carbonyl]amino}propanoic acid

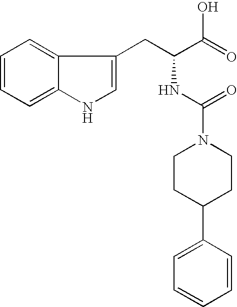

LC/MS (ESI) m/z 392 (M+H⁺).

### Reference Example 69

### ethyl (2R,3S)-2-amino-3-(1H-indol-3-yl)butanoate

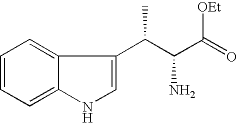

A mixture of ethyl (2R,3S)-2-amino-3-(1H-indol-3-yl)butanoate O,O′-diacetyl-L-tartarate (2.88 g) and a saturated aqueous solution of sodium hydrogen carbonate (35 mL)—ethyl acetate (40 mL) was stirred at room temperature for 2 hrs. The organic layer was separated and the aqueous layer was extracted with ethyl acetate. The obtained organic layer was dried (MgSO₄) and the solvent was evaporated to give the title compound as a colorless oil (1.48 g, yield 100%).

1H NMR (200 MHz, CDCl₃) δ ppm: 1.24 (t, J=7.1 Hz, 3 H), 1.33 (d, J=7.0 Hz, 3 H), 3.58-3.76 (m, 1 H), 3.90 (d, J=4.0 Hz, 1 H), 4.18 (q, J=7.0 Hz, 2 H), 7.05-7.23 (m, 3 H), 7.34-7.42 (m, 1 H), 7.64-7.76 (m, 1 H), 8.06 (s, 1 H).

### Reference Example 70

### ethyl (2R,3S)-2-({[4-(4-chlorophenyl)piperazin-1-yl]carbonyl}amino)-3-(1H-indol-3-yl)butanoate

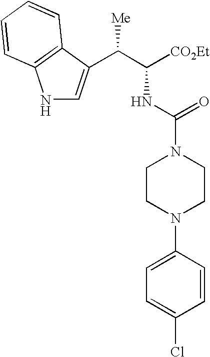

A solution of ethyl (2R,3S)-2-amino-3-(1H-indol-3-yl)butanoate (739 mg, 3.00 mmol) and N,N-diisopropylethylamine (1.03 mL, 6.00 mmol) in acetonitrile (15 mL) was cooled to 0° C. and N,N′-disuccinimidyl carbonate (845 mg, 3.30 mmol) was added by small portions with stirring. After 1 hr., 1-(4-chlorophenyl)piperazine hydrochloride (769 mg, 3.30 mmol) and N,N-diisopropylethylamine (0.567 mL, 3.30 mmol) were added and the mixture was further stirred at room temperature for 3 hrs. The reaction mixture was diluted with ethyl acetate, and washed with water and saturated brine. The organic layer was dried (MgSO₄) and concentrated under reduced pressure. The residue was purified by silica gel column chromatography (developing solvent: ethyl acetate) to give the title compound as a colorless amorphous substance (1.28 g, yield 91%).

¹H NMR (300 MHz, CDCl₃) δ ppm: 1.13 (t, J=7.2 Hz, 3 H), 1.51 (d, J=7.4 Hz, 3 H), 3.01-3.13 (m, 4 H), 3.36-3.52 (m, 4 H), 3.66 (dq, J=5.4, 7.4 Hz, 1 H), 4.07 (q, J 7.2 Hz, 2 H), 4.82 (dd, J=5.4, 8.4 Hz, 1 H), 5.02 (d, J=8.4 Hz, 1 H), 6.78-6.83 (m, 2 H), 7.02 (d, J=2.3 Hz, 1 H), 7.06-7.12 (m, 1 H), 7.15-7.24 (m, 3 H), 7.36 (d, J=8.1 Hz, 1 H), 7.61 (d, J=7.7 Hz, 1 H), 8.10 (s, 1 H).

LC/MS (ESI) m/z 469 (M+H⁺)

The compounds described in the following Reference Examples 71-84 were produced in the similar manner as in Reference Example 70.

### Reference Example 71

### ethyl (2R,3S)-2-({[4-(2-chlorophenyl)piperazin-1-yl]carbonyl}amino)-3-(1H-indol-3-yl)butanoate

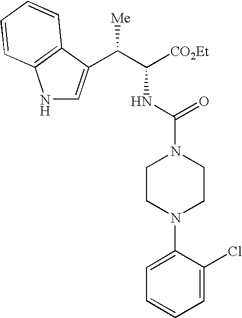

¹H NMR (300 MHz, CDCl₃) δ ppm: 1.13 (t, J=7.2 Hz, 3 H), 1.52 (d, J=7.2 Hz, 3 H), 2.92-3.03 (m, 4 H), 3.40-3.56 (m, 4 H), 3.66 (dq, J=5.5, 7.2 Hz, 1 H), 4.07 (q, J=7.2 Hz, 2 H), 4.83 (dd, J=5.5, 8.3 Hz, 1 H), 5.03 (d, J=8.3 Hz, 1 H), 6.97-7.04 (m, 3 H), 7.08-7.23 (m, 3 H), 7.34-7.38 (m, 2 H), 7.63 (d, J=7.7 Hz, 1 H), 8.09 (brs, 1 H).

LC/MS (ESI) m/z 469 (M+H⁺).

### Reference Example 72

### ethyl (2R,3S)-3-(1H-indol-3-yl)-2-({[4-(4-methylphenyl)piperazin-1-yl]carbonyl}amino)butanoate

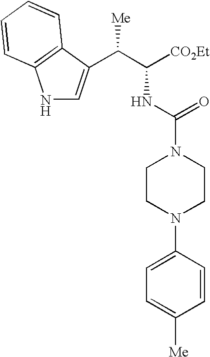

¹H NMR (300 MHz, CDCl₃) δ ppm: 1.12 (t, J=7.2 Hz, 3 H), 1.51 (d, J=7.2 Hz, 3 H), 2.28 (s, 3 H), 3.00-3.12 (m, 4 H), 3.37-3.53 (m, 4 H), 3.65 (dq, J=5.5, 7.2 Hz, 1 H), 4.06 (q, J=7.2 Hz, 2 H), 4.82 (dd, J=5.5, 8.3 Hz, 1 H), 5.03 (d, J=8.3 Hz, 1 H), 6.80-6.84 (m, 2 H), 7.02 (d, J=2.5 Hz, 1 H), 7.06-7.12 (m, 3 H), 7.15-7.20 (m, 1 H), 7.35 (d, J=7.9 Hz, 1 H), 7.62 (d, J=7.9 Hz, 1 H), 8.10 (brs, 1 H).

LC/MS (ESI) m/z 449 (M+H⁺).

### Reference Example 73

### ethyl (2R,3S)-3-(1H-indol-3-yl)-2-[({4-[4-(trifluoromethyl)phenyl]piperazin-1-yl}carbonyl)amino]butanoate

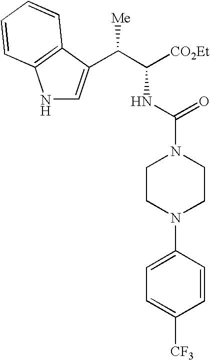

¹H NMR (300 MHz, CDCl₃) δ ppm: 1.14 (t, J=7.2 Hz, 3 H), 1.51 (d, J=7.4 Hz, 3 H), 3.17-3.29 (m, 4 H), 3.38-3.54 (m, 4 H), 3.67 (dq, J=5.4, 7.4 Hz, 1 H), 4.08 (q, J=7.2 Hz, 2 H), 4.82 (dd, J=5.4, 8.4 Hz, 1 H), 5.01 (d, J=8.4 Hz, 1 H), 6.85-6.90 (m, 2 H),-7.03 (d, J=2.3 Hz, 1 H), 7.06-7.11 (m, 1 H), 7.15-7.20 (m, 1 H), 7.36 (d, J 8.1 Hz, 1 H), 7.47-7.51 (m, 2 H), 7.61 (d, J=7.9 Hz, 1 H), 8.09 (s, 1 H).

LC/MS (ESI) m/z 503 (M+H⁺).

### Reference Example 74

### ethyl (2R,3S)-3-(1H-indol-3-yl)-2-({[4-(4-methoxyphenyl)piperazin-1-yl]carbonyl}amino)butanoate

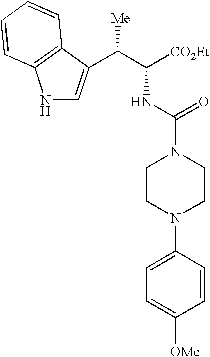

¹H NMR (300 MHz, CDCl₃) δ ppm: 1.12 (t, J=7.2 Hz, 3 H), 1.51 (d, J=7.4 Hz, 3 H), 2.94-3.05 (m, 4 H), 3.37-3.53 (m, 4 H), 3.65 (dq, J=5.5, 7.4 Hz, 1 H), 3.77 (s, 3 H), 4.06 (q, J=7.2 Hz, 2 H), 4.82 (dd, J=5.5, 8.5 Hz, 1 H), 5.03 (d, J=8.5 Hz, 1 H), 6.82-6.91 (m, 4 H), 7.03 (d, J=2.5 Hz, 1 H), 7.07-7.12 (m, 1 H), 7.15-7.20 (m, 1 H), 7.35 (d, J=8.1 Hz, 1 H), 7.61 (d, J=8.1 Hz, 1 H), 8.09 (brs, 1 H).

LC/MS (ESI) m/z 465 (M+H⁺).

### Reference Example 75

### ethyl (2R,3S)-2-({[4-(2-fluorophenyl)piperazin-1-yl]carbonyl}amino)-3-(1H-indol-3-yl)butanoate

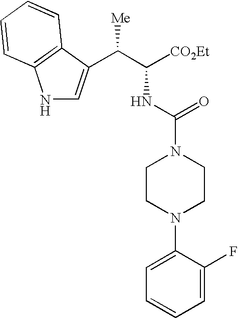

¹H NMR (300 MHz, CDCl₃) δ ppm: 1.13 (t, J 7.1 Hz, 3 H), 1.51 (d, J=7.4 Hz, 3 H), 2.95-3.07 (m, 4 H), 3.40 3.55 (m, 4 H), 3.66 (dq, J=5.5, 7.4 Hz, 1 H), 4.07 (q, J=7.1 Hz, 2 H), 4.82 (dd, J=5.5, 8.4 Hz, 1 H), 5.03 (d, J=8.4 Hz, 1 H), 6.88-7.13 (m, 6 H), 7.15-7.21 (m, 1 H), 7.36 (d, J=8.1 Hz, 1 H), 7.63 (d, J=7.9 Hz, 1 H), 8.09 (brs, 1 H).

LC/MS (ESI) m/z 453 (M+H⁺).

### Reference Example 76

### ethyl (2R,3S)-2-({[4-(4-fluoro-2-methylphenyl)piperazin-1-yl]carbonyl}amino)-3-(1H-indol-3-yl)butanoate

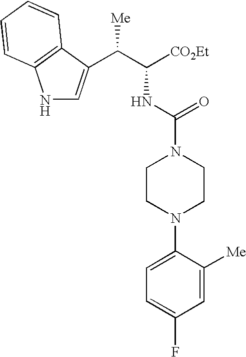

¹H NMR (300 MHz, CDCl₃) δ ppm: 1.13 (t, J=7.2 Hz, 3 H), 1.52 (d, J=7.2 Hz, 3 H), 2.28 (s, 3 H), 2.72-2.84 (m, 4 H), 3.35-3.51 (m, 4 H), 3.66 (dq, J=5.5, 7.2 Hz, 1 H), 4.07 (q, J=7.2 Hz, 2 H), 4.83 (dd, J=5.5, 8.4 Hz, 1 H), 5.02 (d, J=8.4 Hz, 1 H), 6.81-6.95 (m, 3 H), 7.04 (d, J=2.5 Hz, 1 H), 7.08-7.13 (m, 1 H), 7.15-7.21 (m, 1 H), 7.36 (d, J=8.1 Hz, 1 H), 7.63 (d, J=7.5 Hz, 1 H), 8.08 (brs, 1 H).

LC/MS (ESI) m/z 467 (M+H⁺).

### Reference Example 77

### ethyl (2R,3S)-2-({[4-(3-chlorophenyl)piperazin-1-yl]carbonyl}amino)-3-(1H-indol-3-yl)butanoate

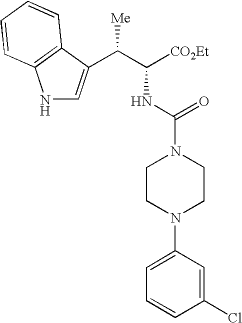

¹H NMR (300 MHz, CDCl₃) δ ppm: 1.14 (t, J=7.2 Hz, 3 H), 1.51 (d, J=7.4 Hz, 3 H), 3.06-3.18 (m, 4 H), 3.36-3.52 (m, 4 H), 3.66 (dq, J=5.5, 7.4 Hz, 1 H), 4.08 (q, J=7.2 Hz, 2 H), 4.82 (dd, J=5.5, 8.3 Hz, 1 H), 5.02 (d, J=8.3 Hz, 1 H), 6.75 (dd, J =1.9, 8.1 Hz, 1 H), 6.82-6.84 (m, 2 H), 7.03 (d, J=2.5 Hz, 1 H), 7.07-7.12 (m, 1 H), 7.14-7.21 (m, 2 H), 7.36 (d, J=8.1 Hz, 1 H), 7.61 (d, J=7.9 Hz, 1 H), 8.10 (brs, 1 H).

LC/MS (ESI) m/z 469 (M+H⁺).

### Reference Example 78

### ethyl (2R,3S)-2-({[4-(3-fluorophenyl)piperazin-1-yl]carbonyl}amino)-3-(1H-indol-3-yl)butanoate

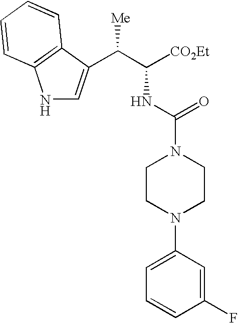

¹H NMR (300 MHz, CDCl₃) δ ppm: 1.13 (t, J=7.2 Hz, 3 H), 1.51 (d, J=7.4 Hz, 3 H), 3.07-3.19 (m, 4 H), 3.36-3.53 (m, 4 H), 3.66 (dq, J=5.5, 7.4 Hz, 1 H), 4.07 (q, J=7.2 Hz, 2 H), 4.81 (dd, J=5.5, 8.4 Hz, 1 H), 5.02 (d, J=8.4 Hz, 1 H), 6.52-6.59 (m, 2 H), 6.64 (ddd, J=0.8, 2.3, 8.3 Hz, 1 H), 7.03 (d, J=2.5 Hz, 1 H), 7.06-7.12 (m, 1 H), 7.16-7.24 (m, 2 H), 7.34-7.37 (m, 1 H), 7.61 (d, J=7.9 Hz, 1 H), 8.10 (brs, 1 H).

LC/MS (ESI) m/z 453 (M+H⁺).

### Reference Example 79

### ethyl (2R,3S)-2-({[4-(4-fluorophenyl)-3-oxopiperazin-1-yl]carbonyl}amino)-3-(1H-indol-3-yl)butanoate

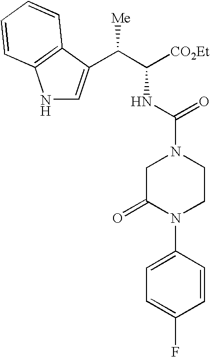

¹H NMR (300 MHz, CDCl₃) δ ppm: 1.17 (t, J=7.2 Hz, 3 H), 1.50 (d, J=7.4 Hz, 3 H), 3.63-3.76 (m, 5 H), 3.98 (d, J=17.1 Hz, 1 H), 4.10 (d, J=17.1 Hz, 1 H), 4.12 (q, J=7.2 Hz, 2 H), 4.80 (dd, J=5.4, 8.2 Hz, 1 H), 4.91 (d, J=8.2 Hz, 1 H), 7.04-7.14 (m, 4 H), 7.17-7.26 (m, 3 H), 7.37 (d, J=8.1 Hz, 1 H), 7.63 (d, J=7.7 Hz, 1 H), 8.10 (brs, 1 H).

LC/MS (ESI) m/z 467 (M+H⁺).

### Reference Example 80

### ethyl (2R,3S)-3-(1H-indol-3-yl)-2-({[4-(2-methylphenyl)-3-oxopiperazin-1-yl]carbonyl}amino)butanoate

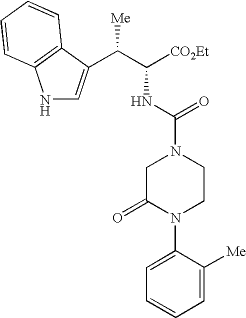

¹H NMR (300 MHz, CDCl₃) δ ppm: 1.16 (t, J=7.2 Hz, 3 H), 1.50 (d, J=7.4 Hz, 3 H), 2.19 (s, 3 H), 3.42-3.53 (m, 1 H), 3.60-3.69 (m, 4 H), 3.98 (dd, J=2.7, 17.0 Hz, 1 H), 4.08-4.15 (m, 3 H), 4.81 (dd, J=5.4, 8.1 Hz, 1 H), 4.95 (d, J=8.1 Hz, 1 H), 7.04 (d, J=2.5 Hz, 1 H), 7.08-7.30 (m, 6 H), 7.36 (d, J=8.1 Hz, 1 H), 7.63 (d, J=7.7 Hz, 1 H), 8.14 (brs, 1 H).

LC/MS (ESI) m/z 463 (M+H⁺).

### Reference Example 81

### ethyl (2R,3S)-2-({[4-(4-fluoro-2-methylphenyl)-3-oxopiperazin-1-yl]carbonyl}amino)-3-(1H-indol-3-yl)butanoate

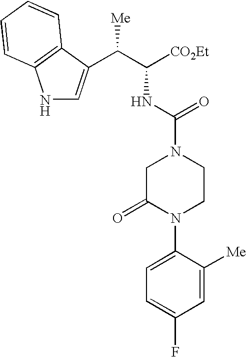

¹H NMR (₃₀₀ MHz, CDCl₃) δ ppm: 1.16 (t, J=7. 1 Hz, 3 H), 1.50 (d, J=7.4 Hz, 3 H), 2.18 (d, J=2.3 Hz, 3 H), 3.41-3.51 (m, 1 H), 3.56-3.87 (m, 4 H), 3.97 (dd, J=4.2, 17.2 Hz, 1 H), 4.07-4.16 (m, 3 H), 4.81 (dd, J=5.4, 8.1 Hz, 1 H), 4.94 (d, J=8.1 Hz, 1 H), 6.90-7.00 (m, 2 H), 7.04-7.15 (m, 3 H), 7.17-7.22 (m, 1 H), 7.37 (d, J=7.9 Hz, 1 H), 7.63 (d, J=7.9 Hz, 1 H), 8.12 (brs, 1 H).

LC/MS (ESI) m/z 481 (M+H⁺).

### Reference Example 82

### ethyl (2R,3S)-2-({[4-(4-fluoro-2-formylphenyl)piperazin-1-yl]carbonyl}amino)-3-(1H-indol-3-yl)butanoate

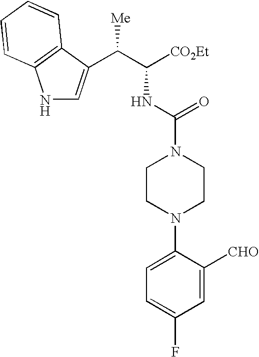

¹H NMR (300 MHz, CDCl₃) δ ppm: 1.15 (t, J=7.2 Hz, 3 H), 1.51 (d, J 7.4 Hz, 3 H), 2.88-3.00 (m, 4 H), 3.40-3.55 (m, 4 H), 3.67 (dq, J=5.5, 7.4 Hz, 1 H), 4.09 (q, J=7.2 Hz, 2 H) 4.81 (dd, J=5.5, 8.3 Hz, 1 H), 5.01 (d, J=8.3 Hz, 1 H), 7.04 (d, J=2.3 Hz, 1 H), 7.07-7.13 (m, 2 H), 7.16-7.21 (m, 1 H), 7.22-7.29 (m, 1 H), 7.36 (d, J=8.1 Hz, 1 H), 7.50 (dd, J=3.1, 8.4 Hz, 1 H), 7.62 (d, J=7.9 Hz, 1 H), 8.11 (brs, 1 H), 10.34 (d, J=3.0 Hz, 1 H).

LC/MS (ESI) m/z 481 (M+H⁺).

### Reference Example 83

### tert-butyl 4-({[(1R,2S)-1-(ethoxycarbonyl)-2-(1H-indol-3-yl)propyl]amino}carbonyl)piperazine-1-carboxylate

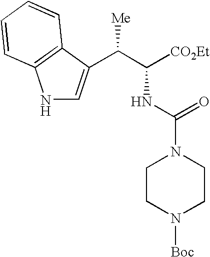

¹H NMR (300 MHz, CDCl₃) δ ppm: 1.13 (t, J=7.2 Hz, 3 H), 1.46 (s, 9 H), 1.50 (d, J=7.4 Hz, 3 H), 3.18-3.42 (m, 8 H), 3.64 (dq, J=5.5, 7.4 Hz, 1 H), 4.07 (q, J=7.2 Hz, 2 H), 4.79 (dd, J=5.5, 8.4 Hz, 1 H), 4.96 (d, J=8.4 Hz, 1 H), 7.02 (d, J=2.5 Hz, 1 H), 7.07-7.12 (m, 1 H), 7.15-7.21 (m, 1 H), 7.34-7.37 (m, 1 H), 7.60 (d, J=7.9 Hz, 1 H), 8.11 (s, H).

LC/MS (ESI) m/z 459 (M+H⁺).

### Reference Example 84

### ethyl (2R,3S)-2-({[4-(anilinocarbonyl)piperazin-1-yl]carbonyl}amino)-3-(1H-indol-3-yl)butanoate

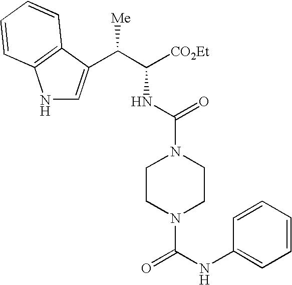

¹H NMR (300 MHz, CDCl₃) δ ppm: 1.15 (t, J=7.2 Hz, 3 H), 1.50 (d, J=7.4 Hz, 3 H), 3.29-3.50 (m, 8 H), 3.67 (dq, J=5.4, 7.2 Hz, 1 H), 4.10 (q, J=7.2 Hz, 2 H), 4.80 (dd, J=5.4, 8.3 Hz, 1 H), 4.96 (d, J=8.3 Hz, 1 H), 6.37 (s, 1 H), 7.01-7.21 (m, 4 H), 7.26-7.36 (m, 5 H), 7.61 (d, J=7.9 Hz, 1 H) 8.19 (s, 1 H).

LC/MS (ESI) m/z 478 (M+H⁺).

The compounds described in the following Reference Examples 85-139 were produced in the similar manner as in Reference Example 2.

### Reference Example 85

### (2R,3S)-3-(1H-indol-3-yl)-2-{[(4-phenylpiperazin-1-yl)carbonyl]amino}butanoic acid

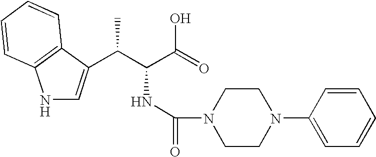

LC/MS (ESI) m/z 407 (M+H⁺).

### Reference Example 86

### (2R,3S)-2-{[(4-benzylpiperidin-1-yl)carbonyl]amino)-3-(1H-indol-3-yl)butanoic acid

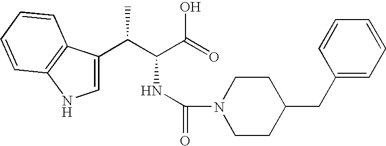

LC/MS (ESI) m/z 420 (M+H⁺).

### Reference Example 87

### (2R,3S)-2-{[(4-benzylpiperazin-1-yl)carbonyl]amino}-3-(1H-indol-3-yl)butanoic acid

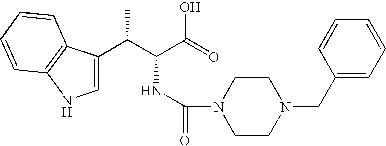

LC/MS (ESI) m/z 421 (M+H⁺).

### Reference Example 88

### (2R,3S)-3-(1H-indol-3-yl)-2-[({4-[2-(trifluoromethyl)phenyl]piperidin-1-yl}carbonyl)amino]butanoic acid

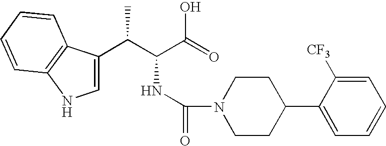

¹H NMR (400 MHz, DMSO-d₆) δ ppm: 1.34 (d, J=7.3 Hz, 3 H), 1.44-1.66 (m, 4 H), 2.67-2.78 (m, 2 H), 2.92-3.03 (m, 1 H), 3.58-3.64 (m, 1 H), 4.04-4.15 (m, 2 H), 4.42-4.46 (m, 1 H), 6.21 (brs, 1 H), 6.94-7.15 (m, 3 H), 7.32 (d, J=8.1 Hz, 1 H), 7.38-7.67 (m, 5 H), 10.79 (s, 1 H).

LC/MS (ESI) m/z 474 (M+H⁺).

### Reference Example 89

### (2R,3S)-3-(1H-indol-3-yl)-2-({[4-(2-methoxyphenyl)piperidin-1-yl]carbonyl}amino)butanoic acid

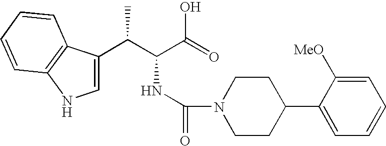

¹H NMR (400 MHz, DMSO-d₆) δ ppm: 1.34 (d, J=7.1 Hz, 3 H), 1.36-1.49 (m, 2 H), 1.61-1.65 (m, 2 H), 2.67-2.77 (m, 2 H), 2.99-3.07 (m, 1 H), 3.54-3.59 (m, 1 H), 3.78 (s, 3 H), 4.02-4.14 (m, 2 H), 4.42-4.46 (m, 1 H), 6.26 (d, J=8.3 Hz, 1 H), 6.88-7.19 (m, 7 H), 7.31 (d, J=8.1 Hz, 1 H), 7.54 (d, J=8.1 Hz, 1 H), 10.91 (s, 1 H).

LC/MS (ESI) m/z 436 (M+H⁺).

### Reference Example 90

### (2R,3S)-2-({[4-(2-fluorophenyl)piperidin-1-yl]carbonyl}amino)-3-(1H-indol-3-yl)butanoic acid

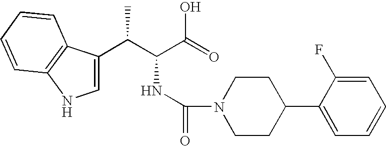

¹H NMR (400 MHz, CDCl₃) δ ppm: 1.32 (d, J=7.0 Hz, 3 H), 1.42-1.57 (m, 4 H), 2.67-2.80 (m, 2 H), 2.93-3.00 (m, 1 H), 3.55-3.59 (m, 1 H), 4.02-4.12 (m, 2 H), 4.37-4.40 (m, 1 H), 6.25 (d, J=8.1 Hz, 1 H), 6.93-7.32 (m, 8 H), 7.31 (d, J=8.1 Hz, 1 H), 7.53 (d, J=7.8 Hz, 1 H), 10.77 (s, 1 H).

LC/MS (ESI) m/z 424 (M+H⁺).

### Reference Example 91

### (2R,3S)-2-({[4-(4-fluorophenyl)-3,6-dihydropyridin-1(2H)-yl]carbonyl}amino)-3-(1H-indol-3-yl)butanoic acid

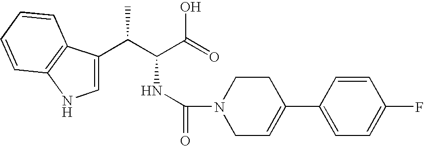

¹H NMR (400 MHz, CDCl₃) δ ppm: 1.51 (d, J=7.3 Hz, 3 H) 2.44-2.49 (m, 2 H), 3.49-3.71 (m, 3 H), 3.63 (s, 3 H), 3.83-3.97 (m, 2 H), 4.86 (dd, J=8.3, 5.4 Hz, 1 H), 4.99 (d, J=8.3 Hz, 1 H), 5.90 (s, 1 H), 7.00-7.20 (m, 5 H), 7.29-7.37 (m, 3 H), 7.62 (d, J=8.1 Hz, 1 H), 8.14 (s, 1 H).

LC/MS (ESI) m/z 436 (M+H⁺).

### Reference Example 92

### (2R,3S)-3-(1H-indol-3-yl)-2-({[4-[2-(trifluoromethyl)phenyl]-3,6-dihydropyridin-1(2H)-yl]carbonyl}amino)butanoic acid

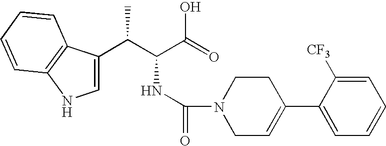

¹H NMR (400 MHz, DMSO-d₆) δ ppm: 1.33 (d, J=7.3 Hz, 3 H), 2.20-2.33 (m, 2 H), 3.46-3.61 (m, 3 H), 3.81-3.98 (m, 2 H), 4.34 (t, J=6.2 Hz, 1 H), 5.57 (s, 1 H), 6.24 (d, J=7.8 Hz, 1 H), 6.94 (t, J=7.3 Hz, 1 H), 7.03 (t, J=7.3 Hz, 1 H) 7.13 (d, J=2.2 Hz, 1 H), 7.30 (d, J=8.3 Hz, 1 H), 7.33 (d, J=7.6 Hz, 1 H), 7.47-7.56 (m, 2 H), 7.64 (t, J=7.4 Hz, 1 H), 7.72 (d, J=7.9 Hz, 1 H).

LC/MS (ESI) m/z 472 (M+H⁺).

### Reference Example 93

### (2R,3S)-2-{[(4-hydroxy-4-phenylpiperidin-1-yl)carbonyl]amino}-3-(1H-indol-3-yl)butanoic acid

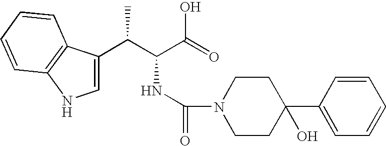

¹H NMR (400 MHz, DMSO-d₆) δ ppm: 1.34 (d, J =7.3 Hz, 3 H), 1.50-1.80 (m, 4 H), 3.04-3.13 (m, 2 H), 3.54-3.62 (m, 1 H), 3.81-3.90 (m, 2 H), 4.49 (dd, J=8.6, 7.1 Hz, 1 H), 5.00 (s, 1 H), 6.26 (d, J=8.5 Hz, 1 H), 6.95-7.07 (m, 2 H), 7.15 (d, J=2.2 Hz, 1 H), 7.19-7.41 (m, 6 H), 7.55 (d, J=7.8 Hz, 1 H), 10.83 (s, 1 H), 12.25 (s, 1 H).

LC/MS (ESI) m/z 422 (M+H⁺).

### Reference Example 94

### (2R,3S)-2-({[4-(4-chlorophenyl)-4-hydroxypiperidin-1-yl]carbonyl}amino)-3-(1H-indol-3-yl)butanoic acid

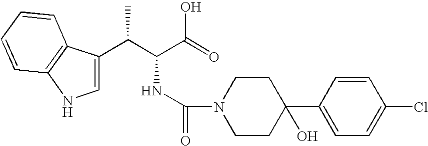

¹H NMR (400 MHz, DMSO-d₆) δ ppm: 1.34 (d, J=7.1 Hz, 3 H), 1.47-1.75 (m, 4 H), 3.02-3.11 (m, 2 H), 3.54-3.61 (m, 1 H), 3.81-4.06 (m, 2 H), 4.49 (dd, J=8.4, 7.1 Hz, 1 H), 5.12 (s, 1 H), 6.26 (d, J=8.4 Hz, 1 H), 6.95-7.07 (m, 2 H), 7.15 (d, J=2.5 Hz, 1 H), 7.34-7.42 (m, 5 H), 7.55 (d, J=7.8 Hz, 1 H), 10.83 (s, 1 H), 12.25 (s, 1 H).

LC/MS (ESI) m/z 456 (M+H⁺).

### Reference Example 95

### (2R,3S)-2-({[4-hydroxy-4-(2-methylphenyl)piperidin-1-yl]carbonyl}amino)-3-(1H-indol-3-yl)butanoic acid

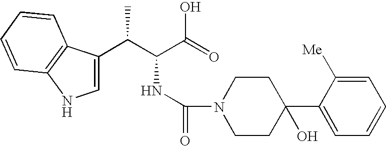

¹H NMR (400 MHz, DMSO-d₆) δ ppm: 1.35 (d, J=7.3 Hz, 3 H), 1.69-1.88 (m, 4 H), 2.47 (s, 3 H), 3.00-3.20 (m, 2 H), 3.53-3.61 (m, 1 H), 3.78-3.87 (m, 2 H), 4.43 (d, J=7.4 Hz, 1 H), 4.88 (s, 1 H), 6.24 (d, J=8.5 Hz, 1 H), 6.93-7.15 (m, 6 H), 7.30-7.35 (m, 2 H), 7.54 (d, J=8.1 Hz, 1 H), 10.80 (s, 1 H).

LC/MS (ESI) m/z 436 (M+H⁺).

### Reference Example 96

### (2R,3S)-3-(1H-indol-3-yl)-2-({[4-(1-naphthyl)piperidin-1-yl]carbonyl}amino)butanoic acid

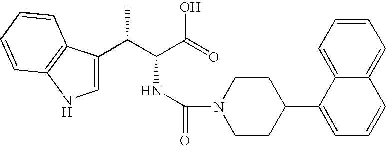

¹H NMR (400 MHz, CDCl₃) δ ppm: 1.36 (d, J=7.1 Hz, 3 H), 1.47-1.65 (m, 2 H), 1.81-1.84 (m, 2 H), 2.90-3.00 (m, 2 H), 3.51-3.61 (m, 2 H), 4.12-4.21 (m, 2 H), 4.49 (t, J=7.7 Hz, 1 H), 6.34 (d, J=8.3 Hz, 1 H), 6.95-7.08 (m, 2 H), 7.16 (d, J=2.2 Hz, 1 H), 7.33 (d, J=7.3 Hz, 2 H), 7.45-7.58 (m, 5 H), 7.78 (d, J=8.1 Hz, 1 H), 7.93 (d, J=8. 3 Hz, 1 H), 8.21 (d, J=8.6 Hz, 1 H), 10. 84 (s, 1 H).

LC/MS (ESI) m/z 456 (M+H⁺).

### Reference Example 97

### (2R,3S)-2-{[(4-benzoylpiperazin-1-yl)carbonyl]amino}-3-(1H-indol-3-yl)butanoic acid

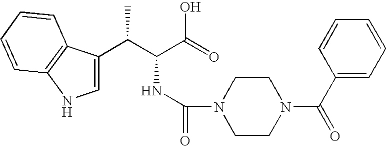

LC/MS (ESI) m/z 435 (M+H⁺).

### Reference Example 98

### (2R,3S)-2-({[4-(4-fluorophenyl)piperidin-1-yl]carbonyl}amino)-3-(1H-indol-3-yl)butanoic acid

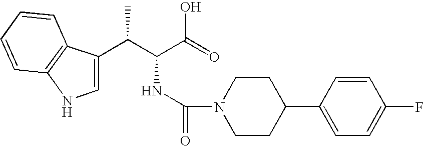

LC/MS (ESI) m/z 424 (M+H⁺).

### Reference Example 99

### (2R,3S)-2-({[4-(4-chlorophenyl)piperidin-1-yl]carbonyl}amino)-3-(1H-indol-3-yl)butanoic acid

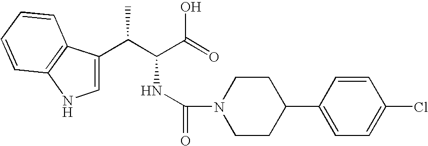

LC/MS (ESI) m/z 440 (M+H⁺).

### Reference Example 100

### (2R,3S)-3-(1H-indol-3-yl)-2-({[4-(4-methylphenyl)piperidin-1-yl]carbonyl}amino)butanoic acid

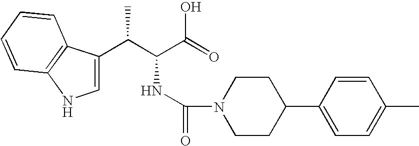

LC/MS (ESI) m/z 420 (M+H⁺).

### Reference Example 101

### (2R,3S)-3-(1H-indol-3-yl)-2-({[4-(4-methoxyphenyl)piperidin-1-yl]carbonyl}amino)butanoic acid

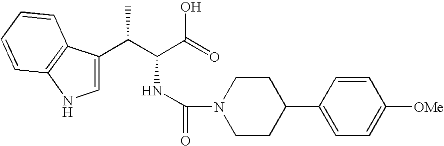

LC/MS (ESI) m/z 436 (M+H⁺).

### Reference Example 102

### (2R,3S)-2-({[4-(2,4-difluorophenyl)piperidin-1-yl]carbonyl}amino)-3-(1H-indol-3-yl)butanoic acid

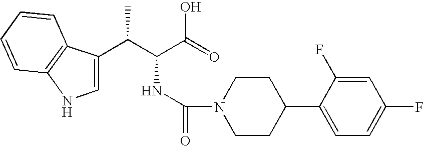

LC/MS (ESI) m/z 442 (M+H⁺).

### Reference Example 103

### (2R,3S)-2-({[4-(3-fluorophenyl)piperidin-1-yl]carbonyl}amino)-3-(1H-indol-3-yl)butanoic acid

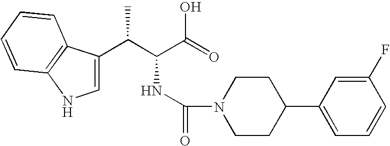

LC/MS (ESI) m/z 424 (M+H⁺).

### Reference Example 104

### (2R,3S)-3-(1H-indol-3-yl)-2-({[4-(3-methylphenyl)piperidin-1-yl]carbonyl}amino)butanoic acid

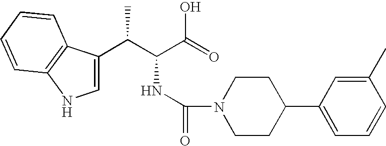

¹H NMR (400 MHz, DMSO-d₆) δ ppm: 1.34 (d, J=7.3 Hz, 3 H), 1.36-1.76 (m, 4 H), 2.28 (s, 3 H), 2.58-2.78 (m, 3 H), 3.55-3.61 (m, 1 H), 4.03-4.14 (m, 2 H), 4.42-4.47 (m, 1 H), 6.27 (d, J=8.5 Hz, 1 H), 6.94-7.21 (m, 8 H), 7.32 (d, J=7.6 Hz, 1 H), 7.54 (d, J=8.1 Hz, 1 H), 10.82 (s, 1 H).

LC/MS (ESI) m/z 420 (M+H⁺).

### Reference Example 105

### (2R,3S)-3-(1H-indol-3-yl)-2-({[4-(3-methoxyphenyl)piperidin-1-yl]carbonyl}amino)butanoic acid

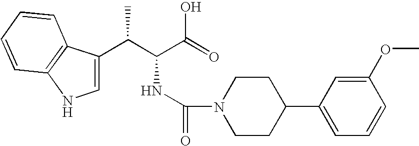

¹H NMR (400 MHz, DMSO-d₆) δ ppm: 1.34 (d, J=7.1 Hz, 3 H), 1.37-1.77 (m, 4 H), 2.60-2.77 (m, 3 H), 3.53-3.60 (m, 1 H), 3.73 (s, 3 H), 4.03-4.15 (m, 2 H), 4.43-4.48 (m, 1 H), 6.29 (d, J=8.3 Hz, 1 H), 6.74-6.82 (m, 4 H), 6.94-7.07 (m, 2 H), 7.15-7.23 (m, 2 H), 7.32 (d, J=8.1 Hz, 1 H), 7.54 (d, J=8.1 Hz, 1 H), 10.83 (s, 1 H).

LC/MS (ESI) m/z 436 (M+H⁺).

### Reference Example 106

### (2R,3S)-3-(1H-indol-3-yl)-2-[({4-[3-(trifluoromethyl)phenyl]piperidin-1-yl)carbonyl)amino]butanoic acid

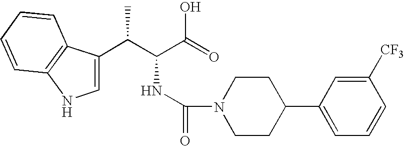

LC/MS (ESI) m/z 474 (M+H⁺).

### Reference Example 107

### (2R,3S)-3-(1H-indol-3-yl)-2-({[4-(2-methylphenyl)piperidin-1-yl]carbonyl}amino)butanoic acid

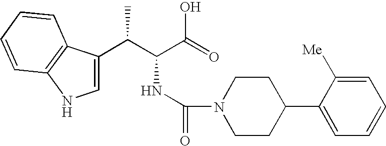

¹H NMR (400 MHz, DMSO-d₆) δ ppm: 1.34 (d, J=7.1 Hz, 3 H), 1.37-1.71 (m, 4 H), 2.31 (s, 3 H), 2.71-2.89 (m, 3 H), 3.55-3.62 (m, 1 H), 4.03-4.13 (m, 2 H), 4.38-4.42 (m, 1 H), 6.24 (d, J=8.6 Hz, 1 H), 6.96 (t, J=7.5 Hz, 1 H), 7.08 (m, 7 H), 7.32 (d, J=8.1 Hz, 1 H), 7.55 (d, J=7.8 Hz, 1 H), 10.79 (s, 1 H).

LC/MS (ESI) m/z 420 (M+H⁺).

### Reference Example 108

### (2R,3S)-2-({[4-(2-ethylphenyl)piperidin-1-yl]carbonyl}amino)-3-(1H-indol-3-yl)butanoic acid

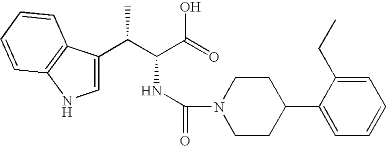

¹H NMR (400 MHz, DMSO-d₆) δ ppm: 1.06 (t, J=7.1 Hz, 3 H), 1.24-1.61 (m, 4 H), 1.34 (d, J=7.1 Hz, 3 H), 2.71-2.88 (m, 3 H), 3.44 (q, J=7.1 Hz, 2 H), 3.56-3.64 (m, 1 H), 4.09 (dd, J=27.8, 13.2 Hz, 2 H), 4.42-4.46 (m, 1 H), 6.24 (d, J=7.6 Hz, 1 H), 6.94-7.15 (m, 8 H), 7.32 (d, J=8.1 Hz, 1 H), 7.55 (d, J=8.1 Hz, 1 H), 10.80 (s, 1 H).

LC/MS (ESI) m/z 434 (M+H⁺).

### Reference Example 109

### (2R,3S)-2-({[4-(4-chlorophenyl)-3,6-dihydropyridin-1(2H)-yl]carbonyl}amino)-3-(1H-indol-3-yl)butanoic acid

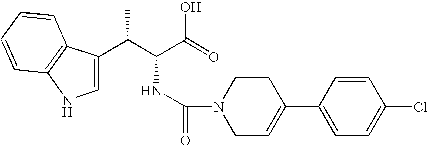

¹H NMR (400 MHz, DMSO-d₆) δ ppm: 1.50 (d, J=6.4 Hz, 3 H), 2.29 (m, 2 H), 3.40-3.50 (m, 2 H), 3.62-3.80 (m, 3 H), 4.71 (brs, 1 H), 5.00 (brs, 1 H), 5.72 (brs, 1 H), 6.99-7.31 (m, 9 H), 7.60 (d, J=7.6 Hz, 1 H), 8.48 (m, 1 H).

LC/MS (ESI) m/z 438 (M+H⁺).

### Reference Example 110

### (2R,3S)-3-(1H-indol-3-yl)-2-({[4-(2-methylphenyl)piperazin-1-yl]carbonyl}amino)butanoic acid

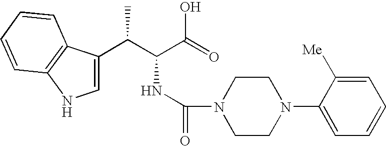

LC/MS (ESI) m/z 421 (M+H⁺).

### Reference Example 111

### (2R,3S)-2-{[(4-cyclohexylpiperazin-1-yl)carbonyl]amino}-3-(1H-indol-3-yl)butanoic acid

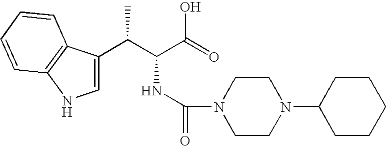

LC/MS (ESI) m/z 413 (M+H⁺).

### Reference Example 112

### (2R,3S)-2-({[4-(4-fluorobenzoyl)piperidin-1-yl]carbonyl}amino)-3-(1H-indol-3-yl)butanoic acid

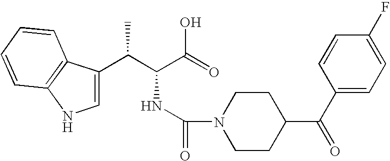

LC/MS (ESI) m/z 452 (M+H⁺).

### Reference Example 113

### (2R,3S)-2-[({4-[(4-fluorophenyl)thio]piperidin-1-yl}carbonyl)amino]-3-(1H-indol-3-yl)butanoic acid

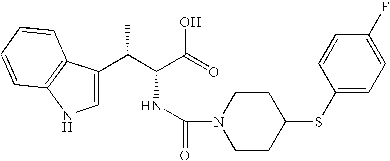

LC/MS (ESI) m/z 456 (M+H⁺).

### Reference Example 114

### (2R,3S)-2-[({4-[(4-fluorophenyl)sulfonyl]piperidin-1-yl}carbonyl)amino]-3-(1H-indol-3-yl)butanoic acid

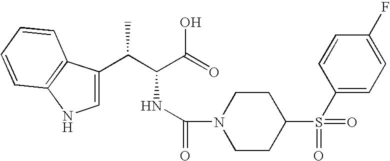

LC/MS (ESI) m/z 488 (M+H⁺).

### Reference Example 115

### (2R,3S)-2-[({4-[(4-fluorophenyl)sulfonyl]piperazin-1-yl}carbonyl)amino]-3-(1H-indol-3-yl)butanoic acid

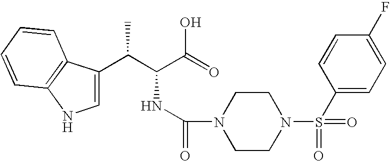

LC/MS (ESI) m/z 489 (M+H⁺).

### Reference Example 116

### (2R,3S)-2-[(3,4-dihydroisoquinolin-2(1H)-ylcarbonyl)amino]-3-(1H-indol-3-yl)butanoic acid

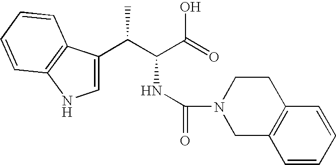

LC/MS (ESI) m/z 378 (M+H⁺).

### Reference Example 117

### (2R,3S)-3-(1H-indol-3-yl)-2-{[(6-methyl-3,4-dihydroisoquinolin-2(1H)-yl)carbonyl]amino}butanoic acid

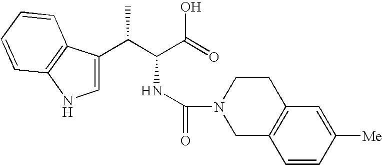

LC/MS (ESI) m/z 392 (M+H⁺).

### Reference Example 118

### (2R,3S)-2-{[(6-chloro-3,4-dihydroisoquinolin-2(1H)-yl)carbonyl]amino}-3-(1H-indol-3-yl)butanoic acid

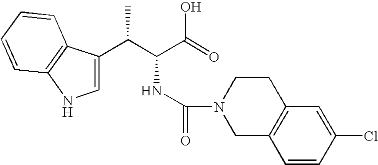

LC/MS (ESI) m/z 412 (M+H⁺).

### Reference Example 119

### (2R,3S)-2-{[(6-fluoro-3,4-dihydroisoquinolin-2(1H)-yl)carbonyl]amino}-3-(1H-indol-3-yl)butanoic acid

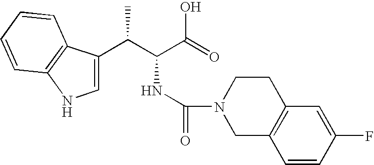

LC/MS (ESI) m/z 396 (M+H⁺).

### Reference Example 120

### (2R,3S)-2-[(4,7-dihydrothieno[2,3-c]pyridin-6(5H)-ylcarbonyl)amino]-3-(1H-indol-3-yl)butanoic acid

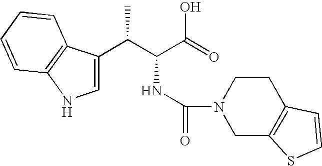

LC/MS (ESI) m/z 384 (M+H⁺).

### Reference Example 121

### (2R,3S)-3-(1H-indol-3-yl)-2-[(1,2,4,5-tetrahydro-3H-3-benzazepin-3-ylcarbonyl)amino]butanoic acid

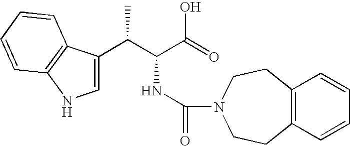

¹H NMR (300 MHz, CDCl₃) δ ppm: 1.56 (d, J=7.3 Hz, 3 H), 2.67-2.85 (m, 4 H), 3.29-3.50 (m, 4 H), 3.70-3.83 (m, 1 H), 4.75 (dd, J=7.7, 5.0 Hz, 1 H), 5.08 (d, J=7.6 Hz, 1 H), 6.97-7.22 (m, 8 H), 7.34 (d, J=8.1 Hz, 1 H), 7.65 (d, J=7.8 Hz, 1 H), 8.31 (s, 1 H).

LC/MS (ESI) m/z 392 (M+H⁺).

### Reference Example 122

### (2R,3S)-2-[(1,3-dihydro-2H-isoindol-2-ylcarbonyl)amino]-3-(1H-indol-3-yl)butanoic acid

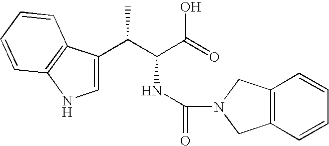

LC/MS (ESI) m/z 364 (M+H⁺).

### Reference Example 123

### (2R,3S)-2-[(2,3-dihydro-1H-indol-1-ylcarbonyl)amino]-3-(1H-indol-3-yl)butanoic acid

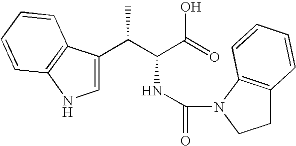

LC/MS (ESI) m/z 364 (M+H⁺).

### Reference Example 124

### (2R,3S)-2-({[4-hydroxy-4-(1,3-thiazol-2-yl)piperidin-1-yl]carbonyl}amino)-3-(1H-indol-3-yl)butanoic acid

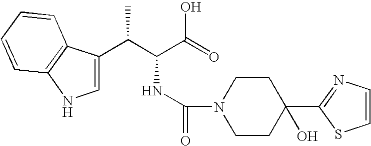

¹H NMR (400 MHz, DMSO-d₆) δ ppm: 1.33 (d, J 7.1 Hz, 3 H), 1.64-2.03 (m, 4 H), 3.07-3.16 (m, 2 H), 3.36 (br, 1 H), 3.51-3.59 (m, 3 H), 4.45 (t, J=7.4 Hz, 1 H), 6.08 (br, 1 H), 6.38 (d, J=8.4 Hz, 1 H), 6.95-7.06 (m, 2 H), 7.15 (d, J=2.0 Hz, 1 H), 7.33 (d, J=7.8 Hz, 1 H), 7.54 (d, J=8.4 Hz, 1 H), 7.59 (d, J=3.3 Hz, 1 H), 7.72 (d, J=3.3 Hz, 1 H) 10.82 (s, 1 H).

LC/MS (ESI) m/z 429 (M+H⁺).

### Reference Example 125

### (2R,3S)-3-(1H-indol-3-yl)-2-({[4-(4-methyl-1,3-thiazol-2-yl)piperidin-1-yl]carbonyl}amino)butanoic acid

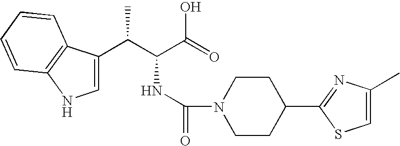

¹H NMR (400 MHz, DMSO-d₆) δ ppm: 1.33 (d, J=7.3 Hz, 3 H), 1.40-1.55 (m, 2 H), 1.90-1.96 (m, 2 H), 2.32 (s, 3 H), 2.76-2.85 (m, 2 H), 3.08-3.15 (m, 1 H), 3.51-3.59 (m, 1 H), 3.99-4.08 (m, 2 H), 4.44 (t, J=8.3 Hz, 1 H), 6.38 (d, J=8.3 Hz, 1 H), 6.94-7.06 (m, 2 H), 7.12 (d, J=1.0 Hz, 1 H), 7.14 (d, J=2.2 Hz, 1 H), 7.31 (d, J=8.1 Hz, 1 H), 7.53 (d, J=8.1 Hz, 1 H), 10.83 (s, 1 H), 12.19 (br, 1 H).

LC/MS (ESI) m/z 427 (M+H⁺).

### Reference Example 126

### (2R,3S)-2-({[4-(4-chlorophenyl)piperazin-1-yl]carbonyl}amino)-3-(1H-indol-3-yl)butanoic acid

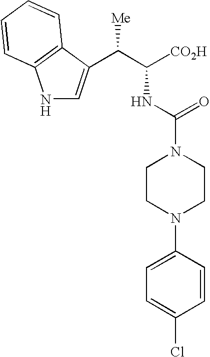

¹H NMR (300 MHz, DMSO-d₆) δ ppm: 1.33 (d, J=7.2 Hz, 3 H), 3.00-3.12 (m, 4 H), 3.37-3.61 (m, 5 H), 4.46 (dd, J=7.4, 8.4 Hz, 1 H), 6.52 (d, J=8.4 Hz, 1 H), 6.93-6.99 (m, 3 H), 7.01-7.07 (m, 1 H), 7.14 (d, J=2.5 Hz, 1 H), 7.21-7.27 (m, 2 H), 7.31 (d, J=7.9 Hz, 1 H), 7.53 (d, J=7.7 Hz, 1 H), 10.82 (d, J=2.5 Hz, 1 H), 12.19 (brs, 1 H).

LC/MS (ESI) m/z 441 (M+H⁺).

### Reference Example 127

### (2R,3S)-2-({[4-(2-chlorophenyl)piperazin-1-yl]carbonyl}amino)-3-(1H-indol-3-yl)butanoic acid

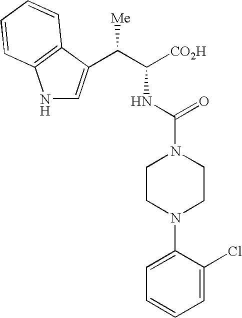

¹H NMR (300 MHz, DMSO-d₆) δ ppm: 1.34 (d, J=7.2 Hz, 3 H), 2.83-2.95 (m,.4 H), 3.39-3.62 (m, 5 H), 4.47 (dd, J=7.2, 8.5 Hz, 1 H), 6.46 (d, J=8.5 Hz, 1 H), 6.94-7.00 (mr 1 H), 7.03-7.09 (m, 2 H), 7.13-7.16 (m, 2 H), 7.28-7.34 (m, 2 H), 7.42 (dd, J=1.5, 7.9 Hz, 1 H), 7.54 (d, J=7.7 Hz, 1 H), 10.83 (d, J=2.5 Hz, 1 H), 12.25 (brs, 1 H).

LC/MS (ESI) m/z 441 (M+H⁺).

### Reference Example 128

### (2R,3S)-3-(1H-indol-3-yl)-2-({[4-(4-methylphenyl)piperazin-1-yl]carbonyl}amino)butanoic acid

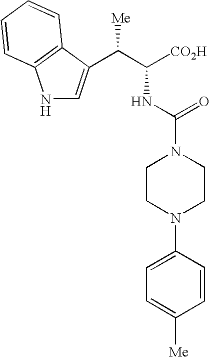

¹H NMR (300 MHz, DMSO-d₆) δ ppm: 1.33 (d, J=7.2 Hz, 3 H), 2.20 (s, 3H), 2.93-3.05 (m, 4 H), 3.37-3.61 (m, 5 H), 4.46 (dd, J=7.4, 8.3 Hz, 1 H), 6.49 (d, J=8.7 Hz, 1 H), 6.83-6.88 (m, 2 H), 6.93-6.99 (m, 3 H), 7.02-7.07 (m, 1 H), 7.14 (d, J=2.3 Hz, 1 H), 7.31 (d, J=7.9 Hz, 1 H), 7.53 (d, J=7.5 Hz, 1 H), 10.82 (d, J=2.3 Hz, 1 H), 12.23 (brs, 1 H).

LC/MS (ESI) m/z 421 (M+H⁺).

### Reference Example 129

### (2R,3S)-3-(1H-indol-3-yl)-2-[({4-[4-(trifluoromethyl)phenyl]piperazin-1-yl}carbonyl)amino]butanoic acid

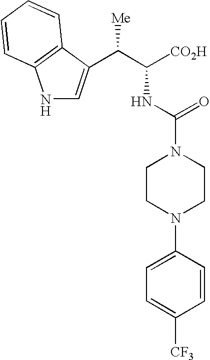

¹H NMR (300 MHz, DMSO-d₆) δ ppm: 1.34 (d, J=7.2 Hz, 3 H), 3.17-3.29 (m, 4 H), 3.40-3.61 (m, 5 H), 4.47 (dd, J=7.4, 8.5 Hz, 1 H), 6.54 (d, J=8.5 Hz, 1 H), 6.93-6.99 (m, 1 H), 7.01-7.10 (m, 3 H), 7.15 (d, J=2.3 Hz, 1 H), 7.32 (d, J=8.1 Hz, 1 H), 7.49-7.56 (m, 3H), 10.82 (d, J=2.3 Hz, 1 H), 12.20 (brs, 1 H).

LC/MS (ESI) m/z 475 (M+H⁺).

### Reference Example 130

### (2R,3S)-3-(1H-indol-3-yl)-2-({[4-(4-methoxyphenyl)piperazin-1-yl]carbonyl}amino)butanoic acid

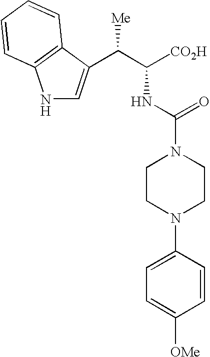

¹H NMR (300 MHz, DMSO-d₆) δ ppm: 1.33 (d, J =7.2 Hz, 3 H), 2.87-2.98 (m, 4 H), 3.37-3.61 (m, 5 H), 3.69 (s, 3 H), 4.46 (dd, J=7.4, 8.2 Hz, 1 H), 6.48 (d, J=8.7 Hz, 1 H), 6.80-6.85 (m, 2 H), 6.89-6.93 (m, 3 H), 7.02-7.07 (m, 1 H), 7.15 (d, J=2.3 Hz, 1 H), 7.32 (d, J=7.9 Hz, 1 H), 7.54 (d, J=7.7 Hz, 1 H), 10.82 (d, J=2.3 Hz, 1 H), 12.22 (brs, 1 H).

LC/MS (ESI) m/z 437 (M+H⁺).

### Reference Example 131

### (2R,3S)-2-({[4-(2-fluorophenyl)piperazin-1-yl]carbonyl}amino)-3-(1H-indol-3-yl)butanoic acid

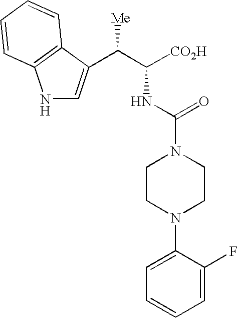

¹H NMR (300 MHz, DMSO-d₆) δ ppm: 1.34 (d, J=7.2 Hz, 3 H), 2.86-2.98 (m, 4 H), 3.39-3.61 (m, 5 H), 4.47 (dd, J=7.4, 8.5 Hz, 1 H), 6.48 (d, J=8.5 Hz, 1 H), 6.94-7.07 (m, 4 H), 7.09-7.18 (m, 3 H), 7.32 (d, J=8.1 Hz, 1 H), 7.54 (d, J=7.5 Hz, 1 H), 10.83 (d, J=1.7 Hz, 1 H), 12.24 (brs, 1 H).

LC/MS (ESI) m/z 425 (M+H⁺).

### Reference Example 132

### (2R,3S)-2-({[4-(4-fluoro-2-methylphenyl)piperazin-1-yl]carbonyl}amino)-3-(1H-indol-3-yl)butanoic acid

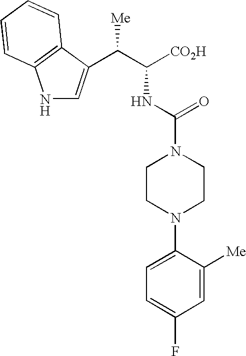

¹H NMR (300 MHz, DMSO-d₆) δ ppm: 1.34 (d, J=7.2 Hz, 3 H), 2.27 (s, 3 H), 2.65-2.76 (m, 4 H), 3.37-3.51 (m, 4 H), 3.57 (pentet, J=7.2 Hz, 1 H), 4.47 (dd, J=7.2, 8.4 Hz, 1 H), 6.44 (d, J=8.4 Hz, 1 H), 6.92-7.08 (m, 5 H), 7.15 (d, J=2.3 Hz, 1 H), 7.32 (d, J=7.9 Hz, 1 H), 7.54 (d, J=7.7 Hz, 1 H), 10.83 (d, J=2.3 Hz, 1 H), 12.23 (brs, 1 H).

LC/MS (ESI) m/z 439 (M+H⁺).

### Reference Example 133

### (2R,3S)-2-({[4-(3-chlorophenyl)piperazin-1-yl]carbonyl}amino)-3-(1H-indol-3-yl)butanoic acid

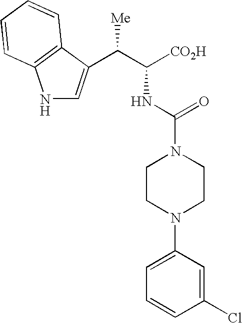

¹H NMR (300 MHz, DMSO-d₆) δ ppm: 1.34 (d, J=7.2 Hz, 3 H), 3.05-3.17 (m, 4 H), 3.37-3.61 (m, 5 H), 4.46 (dd, J=7.4, 8.4 Hz, 1 H), 6.53 (d, J=8.4 Hz, 1 H), 6.80 (dd, J=1.2, 7.8 Hz, 1 H), 6.89-6.99 (m, 3 H), 7.01-7.07 (m, 1 H), 7.14 (d, J=2.3 Hz, 1 H), 7.22 (t, J=8.1 Hz, 1 H), 7.31 (d, J=7.9 Hz, 1 H), 7.53 (d, J=7.7 Hz, 1 H), 10.82 (d, J=2.3 Hz, 1 H), 12.22 (brs, 1 H).

LC/MS (ESI) m/z 441 (M+H⁺).

### Reference Example 134

### (2R,3S)-2-({[4-(3-fluorophenyl)piperazin-1-yl]carbonyl}amino)-3-(1H-indol-3-yl)butanoic acid

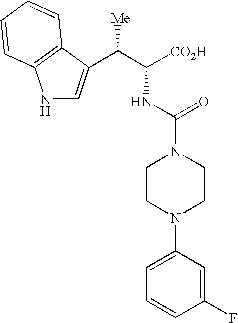

¹H NMR (300 MHz, DMSO-d₆) δ ppm: 1.34 (d, J=7.2 Hz, 3 H), 3.05-3.17 (m, 4 H), 3.39-3.61 (m, 5 H), 4.46 (dd, J=7.4, 8.5 Hz, 1 H), 6.52-6.59 (m, 2 H), 6.73-6.79 (m, 2 H), 6.93-6.99 (m, 1 H), 7.01-7.07 (m, 1 H), 7.14 (d, J=,2.3 Hz, 1 H), 7.18-7.26 (m, 1 H), 7.31 (d, J=8.1 Hz, 1 H), 7.53 (d, J=7.7 Hz, 1 H), 10.82 (d, J=1.5 Hz, 1 H), 12.22 (brs, 1 H).

LC/MS (ESI) m/z 425 (M+H⁺).

### Reference Example 135

### (2R,3S)-2-({[4-(4-fluorophenyl)-3-oxopiperazin-1-yl]carbonyl}amino)-3-(1H-indol-3-yl)butanoic acid

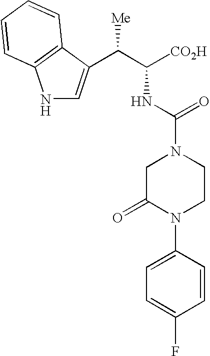

¹H NMR (300 MHz, DMSO-d₆) δ ppm: 1.35 (d, J=7.2 Hz, 3 H), 3.54-3.72 (m, 5 H), 4.03 (d, J=17.5 Hz, 1 H), 4.18 (d, J=17.5 Hz, 1 H), 4.48 (dd, J=7.4, 8.2 Hz, 1 H), 6.63 (d, J=8.7 Hz, 1 H), 6.94-7.00 (m, 1 H), 7.03-7.08 (m, 1 H), 7.17 (d, J=2.3 Hz, 1 H), 7.19-7.27 (m, 2 H), 7.31-7.36 (m, 3 H), 7.54 (d, J=7.7 Hz, 1 H), 10.83 (d, J=1.7 Hz, 1 H), 12.28 (brs, 1 H).

LC/MS (ESI) m/z 439 (M+H⁺).

### Reference Example 136

### (2R,3S)-3-(1H-indol-3-yl)-2-({[4-(2-methylphenyl)-3-oxopiperazin-1-yl]carbonyl}amino)butanoic acid

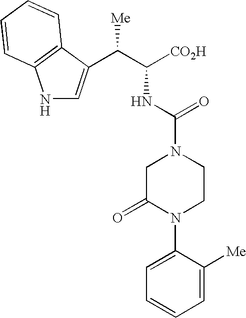

¹H NMR (300 MHz, DMSO-d₆) δ ppm: 1.35 (d, J 7.2 Hz, 3 H), 2.05 (d, J=15.1 Hz, 3 H), 3.30-3.43 (m, 1 H), 3.50-3.84 (m, 4 H), 4.02 (dd, J 7.7, 17.6 Hz, 1 H), 4.18 (dd, J=2.5, 17.6 Hz, 1 H), 4.50 (t, J=7.7 Hz, 1 H), 6.67 (d, J=8.7 Hz, 1 H), 6.95-7.00 (m, 1 H), 7.03-7.08 (m, 1 H), 7.10-7.34 (m, 6 H), 7.54 (d, J=7.7 Hz, 1 H), 10.83 (s, 1 H), 12.29 (brs, 1 H).

LC/MS (ESI) m/z 435 (M+H⁺).

### Reference Example 137

### (2R,3S)-2-({[4-(4-fluoro-2-methylphenyl)-3-oxopiperazin-1-yl]carbonyl}amino)-3-(1H-indol-3-yl)butanoic acid

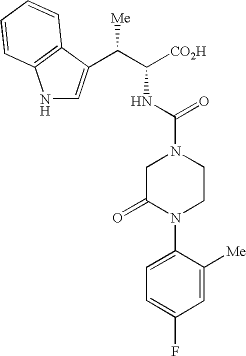

¹H NMR (300 MHz, DMSO-d₆) δ ppm: 1.35 (d, J=7.2 Hz, 3 H), 2.05 (d, J=20.0 Hz, 3 H), 3.35-3.84 -(m, 5 H), 4.01 (dd, J=8.6, 17.6 Hz, 1 H), 4.18 (d, J=17.6 Hz, 1 H), 4.50 (t, J=7.5 Hz, 1 H), 6.67 (d, J=8.7 Hz, 1 H), 6.95-7.00 (m, 1 H), 7.03-7.25 (m, 5 H), 7.29-7.34 (m, 1 H), 7.54 (d, J=7.7 Hz, 1 H), 10.82 (s, 1 H), 12.29 (brs, 1 H).

LC/MS (ESI) m/z 453 (M+H⁺).

### Reference Example 138

### (2R,3S)-2-({[4-(4-fluoro-2-formylphenyl)piperazin-1-yl]carbonyl}amino)-3-(1H-indol-3-yl)butanoic acid

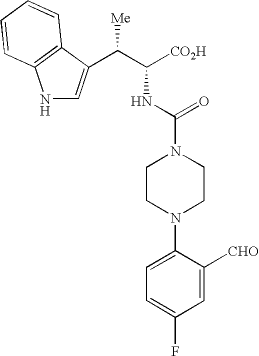

¹H NMR (300 MHz, CDCl₃) δ ppm: 1.55 (d, J=7.4 Hz, 3 H), 2.87-2.99 (m, 4 H), 3.38-3.55 (m, 4 H), 3.76 (dq, J=4.8, 7.4 Hz, 1 H), 4.80 (dd, J=4.8, 7.7 Hz, 1 H), 5.13 (d, J=7.7 Hz, 1 H), 7.05-7.18 (m, 4 H), 7.23-7.29 (m, 1 H), 7.35 (d, J=8.1 Hz, 1 H), 7.50 (dd, J=3.2, 8.5 Hz, 1 H), 7.64 (d, J=7.7 Hz, 1 H), 8.60 (brs, 1 H), 10.33 (d, J=2.8 Hz, 1 H).

LC/MS (ESI) m/z 453 (M+H⁺).

### Reference Example 139

### (2R,3S)-2-({[4-(anilinocarbonyl)piperazin-1-yl]carbonyl}amino)-3-(1H-indol-3-yl)butanoic acid

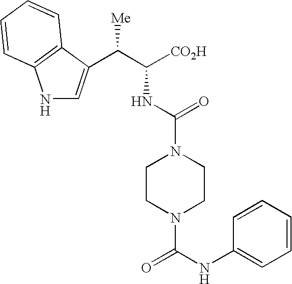

¹H NMR (300 MHz, DMSO-d₆) δ ppm: 1.34 (d, J=7.4 Hz, 3 H), 3.27-3.42 (m, 8 H), 3.56 (pentet, J=7.4 Hz, 1 H), 4.47 (dd, J=7.4, 8.5 Hz, 1 H), 6.48 (d, J=8.5 Hz, 1 H), 6.91-7.00 (m, 2 H), 7.02-7.07 (m, 1 H), 7.15 (d, J 2.5 Hz, 1 H), 7.20-7.26 (m, 2 H), 7.54 (d, J=7.5 Hz, 1 H), 8.53 (s, 1 H), 10.83 (d, J=2.5 Hz, 1 H), 12.10 (brs, 1 H).

LC/MS (ESI) m/z 450 (M+H⁺).

### Reference Example 140

### ethyl (2R,3S)-2-({[4-(cyclopropylmethyl)piperazin-1-yl]carbonyl}amino)-3-(1H-indol-3-yl)butanoate

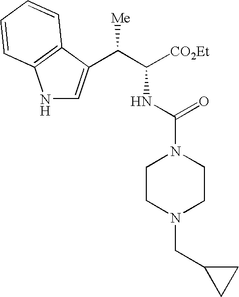

To a solution of tert-butyl 4-({[(1R,2S)-1-(ethoxycarbonyl)-2-(1H-indol-3-yl)propyl]amino}carbonyl)piperazine-1-carboxylate (2.25 g, 4.91 mmol) in ethyl acetate (20 mL) was added 4N hydrochloric acid-ethyl acetate (20 mL) and the mixture was stirred at room temperature for 1 hr. To the reaction mixture was added 1N aqueous sodium hydroxide solution (150 mL) and the mixture was extracted with ethyl acetate. The organic layer was washed with saturated brine, dried (Na₂SO₄) and concentrated under reduced pressure.

A suspension of the obtained residue, (bromomethyl)cyclopropane (0.715 mL, 7.37 mmol) and potassium carbonate (1.36 g, 9.82 mmol) in DMF (20 mL) was stirred at room temperature for 3 hrs. The reaction mixture was diluted with ethyl acetate, and washed twice with water and once with saturated brine. The organic layer was dried (Na₂SO₄) and concentrated under reduced pressure. The residue was purified by aminopropyl silica gel column chromatography (developing solvent: ethyl acetate) to give the title compound as a colorless amorphous substance (1.15 g, yield 57%).

¹H NMR (300 MHz, CDCl₃) δ ppm: 0.02-0.17 (m, 2 H), 0.44-0.60 (m, 2 H), 0.78-0.91 (m, 1 H), 1.10 (t, J =7.2 Hz, 3 H), 1.50 (d, J=7.2 Hz, 3 H), 2.25 (d, J=6.6 Hz, 2 H), 2.40-2.52 (m, 4 H), 3.27-3.42 (m, 4 H), 3.62 (dq, J=5.5, 7.2 Hz, 1 H), 4.04 (q, J=7.2 Hz, 2 H), 4.80 (dd, J=5.5, 8.3 Hz, 1 H), 4.98 (d, J=8.3 Hz, 1 H), 7.02 (d, J=2.5 Hz, 1 H), 7.07-7.12 (m, 1 H), 7.15-7.20 (m, 1 H), 7.35 (d, J=8.1 Hz, 1 H), 7.61 (d, J=7.7 Hz, 1 H), 8.09 (s, 1 H).

LC/MS (ESI) m/z 413 (M+H⁺).

### Reference Example 141

### (2R,3S)-2-({[4-(cyclopropylmethyl)piperazin-1-yl]carbonyl}amino)-3-(1H-indol-3-yl)butanoic acid

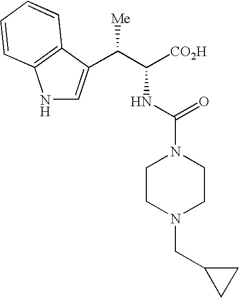

A mixed solution of ethyl (2R,3S)-2-({[4-(cyclopropylmethyl)piperazin-1-yl]carbonyl}amino)-3-(1H-indol-3-yl)butanoate (1.15 g, 2.79 mmol), 1N aqueous sodium hydroxide solution (10 mL) and ethanol (20 mL) was stirred overnight at room temperature. The reaction mixture was poured into water and neutralized by adding 1N hydrochloric acid (10 mL). This aqueous solution was saturated with sodium chloride and extracted with ethyl acetate. The organic layer was dried (Na₂SO₄) and concentrated under reduced pressure to give the title compound as a pale yellow amorphous substance (0.737 g, yield 69%).

¹H NMR (300 MHz, DMSO-d₆) δ ppm: 0.30-0.41 (m, 2 H), 0.57-0.70 (m, 2 H), 1.02-1.14 (m, 1 H), 1.33 (d, J=7.2 Hz, 3 H), 2.55-3.60 (m, 11 H), 4.46 (dd, J=7.4, 8.4 Hz, 1 H), 6.73 (d, J=8.1 Hz, 1 H), 6.94-6.99 (m, 1 H), 7.02-7.07 (m, 1 H), 7.14 (d, J 2.3 Hz, 1 H), 7.32 (d, J=7.9 Hz, 1 H), 7.53 (d, J=7.7 Hz, 1 H), 10.86 (d, J =1.9 Hz, 1 H).

LC/MS (ESI) m/z 385 (M+H⁺).

### Reference Example 142

### 4-methoxybenzyl (2R,3S)-2-{[(9H-fluoren-9-ylmethoxy)carbonyl]amino}-3-(1H-indol-3-yl)butanoate

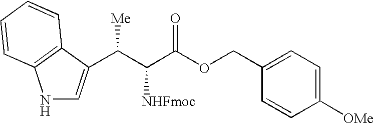

A suspension of (2R,3S)-2-{([(9H-fluoren-9-ylmethoxy)carbonyl]amino}-3-(1H-indol-3-yl)butanoic acid (2.20 g, 5.00 mmol), 4-methoxybenzyl chloride (1.02 mL, 7.50 mmol) and potassium carbonate (1.04 g, 7.50 mmol) in DMF (25 mL) was stirred at room temperature for 6 hrs. The reaction mixture was diluted with ethyl acetate, and washed twice with water and once with saturated brine. The organic layer was dried (MgSO₄) and concentrated under reduced pressure. The residue was recrystallized from hexane/THF to give the title compound as colorless prism crystals (2.19 g, yield 78%).

¹H NMR (300 MHz, CDCl₃) δ ppm: 1.42 (d, J=7.4 Hz, 3 H), 3.56-3.65 (m, 1 H), 3.79 (s, 3 H), 4.18 (t, J=6.9 Hz, 1 H), 4.29-4.41 (m, 2 H), 4.72 (dd, J=6.0, 9.3 Hz, 1 H), 4.94 (s, 2 H), 5.37 (d, J=9.3 Hz, 1 H), 6.78-6.82 (m, 2 H), 6.87 (s, 1 H), 7.04-7.14 (m, 3 H), 7.20 (t, J=7.4 Hz, 1 H), 7.26-7.42 (m, 5 H), 7.54-7.63 (m, 3 H), 7.76 (d, J=7.5 Hz, 2 H), 7.93 (brs, 1 H).

LC/MS (ESI) m/z 561 (M+H⁺).

### Reference Example 143

### 4-methoxybenzyl (2R,3S)-2-amino-3-(1H-indol-3-yl)butanoate

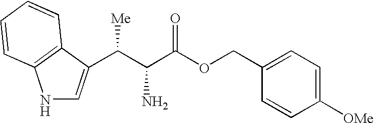

A mixed solution of 4-methoxybenzyl (2R,3S)-2-{[(9H-fluoren-9-ylmethoxy)carbonyl]amino)-3-(1H-indol-3-yl)butanoate (2.07 g, 3.70 mmol), piperidine (1 mL) and DMF (20 mL) was stirred at room temperature for 1 hr. The reaction mixture was diluted with ethyl acetate, and washed twice with water and once with saturated brine. The organic layer was dried (MgSO₄) and concentrated under reduced pressure. The residue was purified by silica gel column chromatography (developing solvent: hexane/ethyl acetate=1/1-0/1) to give the title compound as a colorless oil (1.16 g, yield 93%).

¹H NMR (300 MHz, CDCl₃) δ ppm: 1.29 (d, J=7.2 Hz, 3 H), 1.38 (brs, 2 H), 3.62 (ddq, J=0.8, 4.5, 7.2 Hz, 1 H), 3.81 (s, 3 H), 3.91 (d, J=4.5 Hz, 1 H), 5.06 (s, 2 H), 6.84-6.88 (m, 2 H), 7.02 (d, J=1.9 Hz, 1 H), 7.11 (ddd, J=1.2, 7.0, 8.0 Hz, 1 H), 7.17-7.23 (m, 3 H), 7.36 (td, J=1.2, 8.0 Hz, 1 H), 7.66 (d, J=8.0 Hz, 1 H), 8.04 (brs, 1 H).

LC/MS (ESI) m/z 339 (M+H⁺).

### Reference Example 144

### 4-methoxybenzyl (2R,3S)-2-{[(3,5-dioxo-4-phenylpiperazin-1-yl)carbonyl]amino}-3-(1H-indol-3-yl)butanoate

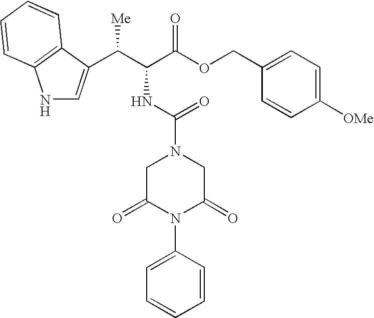

A solution of 4-methoxybenzyl (2R,3S)-2-amino-3-(1H-indol-3-yl)butanoate (1.15 g, 3.40 mmol) and N,N-diisopropylethylamine (1.17 mL, 6.80 mmol) in acetonitrile (15 mL) was cooled to 0° C. Thereto was added N,N′-disuccinimidyl carbonate (845 mg, 3.30 mmol) by small portions with stirring. After 1 hr., 2,6-dioxo-1-phenylpiperazine (0.711 g, 3.74 mmol) was added and the mixture was stirred at room temperature for 18 hrs. The reaction mixture was diluted with ethyl acetate, and washed successively with 1N hydrochloric acid, saturated aqueous solution of sodium hydrogen carbonate and saturated brine. The organic layer was dried (MgSO₄) and concentrated under reduced pressure. The residue was purified by silica gel column chromatography (developing solvent: hexane/ethyl acetate=1/1-0/1) to give the title compound as a colorless amorphous substance (1.61 g, yield 85%).

¹H NMR (300 MHz, CDCl₃) δ ppm: 1.43 (d, J=7.2 Hz, 3 H), 3.69 (dq, J=5.3, 7.2 Hz, 1 H), 3.82 (s, 3 H), 4.25 (d, J=18.1 Hz, 2 H), 4.34 (d, J=18.1 Hz, 2 H), 4.82 (dd, J=5.3, 8.3 Hz, 1 H), 5.00 (d, J=11.9 Hz, 1 H), 5.07-5.11. (m, 2 H), 6.78 (d, J=2.5 Hz, 1 H), 6.84-6.89 (m, 2 H), 7.05-7.15 (m, 3 H), 7.17-7.23 (m, 3 H), 7.35 (d, J=7.9 Hz, 1 H), 7.40-7.50 (m, 3 H), 7.59 (d, J=7.9 Hz, 1 H), 8.00 (brs, 1 H).

LC/MS (ESI) m/z 555 (M+H⁺).

### Reference Example 145

### (2R,3S)-2-{[(3,5-dioxo-4-phenylpiperazin-1-yl)carbonyl]amino}-3-(1H-indol-3-yl)butanoic acid

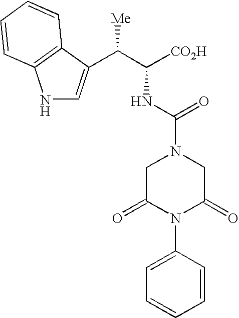

A suspension of 4-methoxybenzyl (2R,3S)-2-{[(3,5-dioxo-4-phenylpiperazin-1-yl)carbonyl]amino}-3-(1H-indol-3-yl)butanoate (1.60 g, 2.88 mmol) and 5% palladium-carbon (1.60 g) in methanol (50 mL) was stirred under a hydrogen atmosphere at room temperature for 1 hr. The reaction mixture was filtered, and the filtrate was concentrated under reduced pressure. The residue was purified by silica gel column chromatography (developing solvent: ethyl acetate) to give the title compound as a pale red amorphous substance (0.645 g, yield 52%).

¹H NMR (300 MHz, CDCl₃) δ ppm: 1.49 (d, J=7.2 Hz, 3 H), 3.71-3.81 (m, 1 H), 4.09 (d, J=18.1 Hz, 2 H), 4.21 (d, J=18.1 Hz, 2 H), 4.78 (dd, J=5.7, 7.2 Hz, 1 H), 5.46 (d, J =7.2 Hz, 1 H), 6.90-6.92 (m, 2 H), 7.02 (s, 1 H), 7.11-7.20 (m, 2 H), 7.26 (d, J=7.4 Hz, 1 H), 7.36-7.39 (m, 3 H), 7.63 (d, J=7.4 Hz, 1 H), 8.31 (brs, 1 H).

LC/MS (ESI) m/z 435 (M+H⁺).

### Reference Example 146

### tert-butyl 4-(4-fluoro-2-methylphenyl)piperazine-1-carboxylate

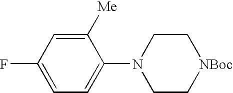

To a solution of bis(dibenzylidene acetone) palladium(0) (0.719 g, 1.25 mmol) and tris(2-methylphenyl)phosphine (0.761 g, 2.50 mmol) in toluene (150 mL) were added sodium tert-butoxide (3.36 g, 35.0 mmol), 2-bromo-5-fluorotoluene (3.16 mL, 25.0 mmol) and tert-butyl 1-piperazinecarboxylate (5.03 g, 27.0 mmol) at room temperature, and the mixture was stirred under a nitrogen atmosphere at 100° C. for 20 hrs. After cooling, the reaction mixture was washed with water and saturated brine, dried (MgSO₄) and concentrated under reduced pressure. The residue was purified by silica gel column chromatography (developing solvent: hexane/ethyl acetate=9/1) to give the title compound as a yellow oil (2.50 g, yield 34%).

¹H NMR (300 MHz, CDCl₃) δ ppm: 1.49 (s, 9 H), 2.30 (s, 3 H), 2.77-2.80 (m, 4 H), 3.54-3.57 (m, 4 H), 6.80-6.96 (m, 3 H).

LC/MS (ESI) m/z 295 (M+H⁺).

### Reference Example 147

### 1-(4-fluord-2-methylphenyl)piperazine dihydrochloride

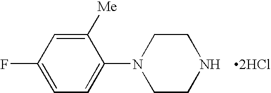

A mixed solution of tert-butyl 4-(4-fluoro-2-methylphenyl)piperazine-1-carboxylate (2.50 g, 8.50 mmol) and 5% hydrogen chloride methanol solution (50 mL) was heated under reflux for 1 hr. After cooling, the reaction mixture was concentrated under reduced pressure, and the residue was recrystallized from ethanol to give the title compound (2.16 g, 95%) as colorless prism crystals.

¹H NMR (300 MHz, DMSO-d₆) δ ppm: 2.26 (s, 3 H), 3.00-3.03 (m, 4 H), 3.16-3.22 (m, 4 H), 6.95-7.09 (m, 3 H), 9.38 (brs, 2 H).

LC/MS (ESI) m/z 195 (M+H⁺) −2HCl.

### Reference Example 148

### 2-chloro-N-(4-fluorophenyl)acetamide

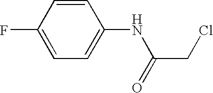

A mixture of 4-fluoroaniline (9.47 mL, 100 mmol), isopropyl acetate (100 mL) and 2N aqueous potassium hydrogen carbonate solution (100 mL) was cooled to 0° C. and chloroacetyl chloride (9.56 mL, 120 mmol) was added dropwise. The mixture was stirred at room temperature for 30 min. The aqueous layer was separated and ethyl acetate was added. The precipitated crystals were dissolved. This solution was washed with saturated brine, dried (MgSO₄) and concentrated under reduced pressure. The residue was recrystallized from hexane/ethyl acetate to give the title compound as colorless prism crystals (17.9 g, yield 95%).

¹H NMR (300 MHz, CDCl₃) δ ppm: 4.20 (s, 2 H), 7.02-7.10 (m, 2 H), 7.48-7.55 (m, 2 H), 8.21 (brs, 1 H).

LC/MS (ESI) m/z 188 (M+H⁺).

The compounds described in the following Reference Examples 149-151 were produced in the similar manner as in Reference Example 148.

### Reference Example 149

### 2-chloro-N-(2-methylphenyl)acetamide

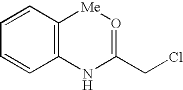

¹H NMR (300 MHz, CDCl₃) δ ppm: 2.31 (s, 3 H), 4.24 (s, 2 H), 7.10-7.15 (m, 1 H), 7.21-7.27 (m, 2 H), 7.88 (d, J=8.3 Hz, 1 H), 8.23 (brs, 1 H).

LC/MS (ESI) m/z 184 (M+H⁺).

### Reference Example 150

### 2-chloro-N-(4-fluoro-2-methylphenyl)acetamide

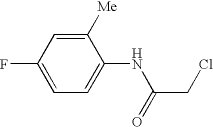

¹H NMR (300 MHz, CDCl₃) δ ppm: 2.29 (s, 3 H), 4.24 (s, 2 H), 6.90-6.96 (m, 2 H), 7.72-7.77 (m, 1 H), 8.12 (brs, 1 H).

LC/MS (ESI) m/z 202 (M+H⁺).

### Reference Example 151

### N-(2-bromo-4-fluorophenyl)-2-chloroacetamide

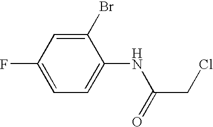

¹H NMR (300 MHz, CDCl₃) δ ppm: 4.24 (s, 2 H), 7.08 (ddd, J=2.8, 7.8, 9.1 Hz, 1 H), 7.34 (dd, J=2.8, 7.8 Hz, 1 H), 8.31 (dd, J=5.6, 9.1 Hz, 1 H), 8.82 (brs, 1 H).

### Reference Example 152

### N-(4-fluorophenyl)-2-(2-hydroxyethylamino)acetamide

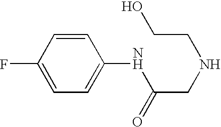

A mixture of 2-chloro-N-(4-fluorophenyl)acetamide (9.38 g, 50.0 mmol), 2-aminoethanol (12.1 mL, 200 mmol) and isopropyl acetate (50 mL) was stirred at 60° C. for 1.5 hrs. After cooling, the reaction mixture was poured into water, and extracted three times with ethyl acetate. The organic layer was dried (MgSO₄) and concentrated under reduced pressure. The residue was washed with ethyl acetate to give the title compound as colorless prism crystals (8.55 g, yield 81%).

¹H NMR (300 MHz, DMSO-d₆) δ ppm: 2.41 (brs, 1 H), 2.61 (t, J=5.4 Hz, 2 H), 3.28 (s, 2 H), 3.46 (q, J=5.4 Hz, 2 H), 4.63 (t, J=5.4 Hz, 1 H), 7.10-7.18 (h, 2 H), 7.61-7.69 (m, 2 H), 9.94 (brs, 1 H).

LC/MS (ESI) m/z 213 (M+H⁺).

The compounds described in the following Reference Examples 153-155 were produced in the similar manner as in Reference Example 152.

### Reference Example 153

### 2-[(2-hydroxyethyl)amino]-N-(2-methylphenyl)acetamide

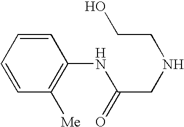

1H NMR (300 MHz, CDCl₃) δ ppm: 2.10 (brs, 2 H), 2.28 (s, 3 H), 2.83-2.87 (m, 2 H), 3.44 (s, 2 H), 3.73-3.77 (m, 2 H), 7.05 (dt, J=1.0, 7.4 Hz, 1 H), 7.16-7.24 (m, 2 H), 8.04 (d, J=8.1 Hz, 1 H), 9.33 (brs, 1 H).

LC/MS (ESI) m/z 209 (M+H⁺).

### Reference Example 154

### N-(4-fluoro-2-methylphenyl)-2-(2-hydroxyethylamino)acetamide

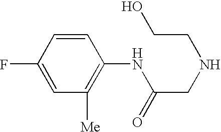

¹H NMR (300 MHz, CDCl₃) δ ppm: 1.92 (brs, 2 H), 2.27 (s, 3 H), 2.85-2.88 (m, 2 H), 3.45 (s, 2 H), 3.75-3.78 (m, 2 H), 6.87-6.94 (m, 2 H), 7.90-7.95 (m, 1 H), 9.24 (brs, 1 H).

LC/MS (ESI) m/z 227 (M+H⁺).

### Reference Example 155

### N-(2-bromo-4-fluorophenyl)-2-(2-hydroxyethylamino)acetamide

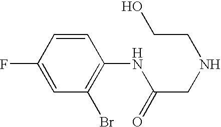

¹H NMR (300 MHz, CDCl₃) δ ppm: 1.72 (brs, 1 H), 2.10 (brs, 1 H), 2.87-2.91 (m, 2 H), 3.48 (s, 2 H), 3.80-3.83 (m, 2 H), 7.06 (ddd, J=2.9, 7.8, 9.2 Hz, 1 H), 7.30.(dd, J=2.9, 7.8 Hz, 1 H), 8.43 (dd, J=5.7, 9.2 Hz, 1 H), 9.88 (brs, 1 H).

### Reference Example 156

### 1-(4-fluorophenyl)piperazin-2-one hydrochloride

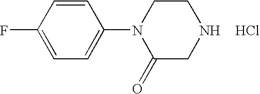

To a solution of N-(4-fluorophenyl)-2-(2-hydroxyethylamino)acetamide (4.14 g, 20.0 mmol) and tributylphosphine (5.98 mL, 24.0 mmol) in THF (100 mL) was added di-tert-butyl azodicarboxylate (5.08 g, 25.0 mmol) by small portions with stirring at room temperature. After 1 hr., 10% hydrogen chloride—methanol solution (15 mL) was added and the mixture was concentrated under reduced pressure. The residue was washed with ethyl acetate and recrystallized from ethanol to give the title compound as colorless prism crystals (2.02 g, yield 44%).

¹H NMR (300 MHz, DMSO-d₆) δ ppm: 3.50-3.53 (m, 2 H), 3.84-3.87 (m, 4 H), 7.24-7.32 (m, 2 H), 7.33-7.40 (m, 2 H), 9.81 (s, 2 H).

LC/MS (ESI) m/z 195 (M+H⁺) —HCl.

The compounds described in the following Reference Examples 157-158 were produced in the similar manner as in Reference Example 156.

### Reference Example 157

### 1-(2-methylphenyl)piperazin-2-one hydrochloride

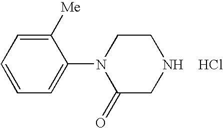

¹H NMR (300 MHz, DMSO-d₆) δ ppm: 2.17 (s, 3 H), 3.47-3.60 (m, 2 H), 3.63-3.70 (m, 1 H), 3.78-3.86 (m, 2 H), 3.91 (d, J=16.6 Hz, 1 H), 7.17-7.23 (m, 1 H), 7.25-7.33 (m, 3 H), 9.90 (brs, 2 H).

LC/MS (ESI) m/z 191 (M+H⁺) —HCl.

### Reference Example 158

### 1-(4-fluoro-2-methylphenyl)piperazin-2-one hydrochloride

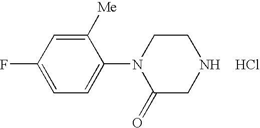

¹H NMR (300 MHz, DMSO-d₆) δ ppm: 2.17 (s, 3 H), 3.47-3.60 (m, 2 H), 3.62-3.70 (m, 1 H), 3.75-3.84 (m, 2 H), 3.92 (d, J=16.6 Hz, 1 H), 7.08-7.20 (m, 2 H), 7.25 (dd, J 5.7, 8.7 Hz, 1 H), 9.85 (brs, 2 H).

LC/MS (ESI) m/z 209 (M+H⁺) —HCl.

### Reference Example 159

### tert-butyl 4-(2-bromo-4-fluorophenyl)-3-oxopiperazine-1-carboxylate

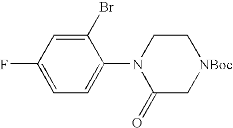

To a solution of N-(2-bromo-4-fluorophenyl)-2-(2-hydroxyethylamino)acetamide (12.8 g, 44.0 mmol) and tributylphosphine (13.2 mL, 52.8 mmol) in THF (100 mL) was added dropwise diisopropyl azodicarboxylate (10.8 mL, 55.0 mmol) with stirring at 0° C. and the mixture was stirred at 60° C. for 1 hr. After cooling, 10% hydrogen chloride-methanol solution (50 mL) was added and the mixture was concentrated under reduced pressure. The residue was crystallized from hexane/ethyl acetate to give crude 1-(2-bromo-4-fluorophenyl)piperazin-2-one hydrochloride as colorless crystals.

To a mixture of the obtained hydrochloride, ethyl acetate (150 mL) and a saturated aqueous solution of sodium hydrogen carbonate solution (150 mL) was added di-tert-butyl dicarbonate (10.1 mL, 44.0 mmol) at room temperature and the mixture was stirred for 30 min. The organic layer was washed with saturated brine, dried (MgSO₄) and concentrated under reduced pressure. The residue was purified by silica gel column chromatography (developing solvent: hexane/ethyl acetate=1/1) to give the title compound as colorless crystals (10.7 g, yield 65%). Recrystallization from hexane/ethyl acetate gave colorless prism crystals.

¹H NMR (300 MHz, CDCl₃) δ ppm: 1.50 (s, 9 H), 3.53-3.77 (m, 3 H), 3.90-3.98 (m, 1 H), 4.19 (d, J=18.5 Hz, 1 H), 4.35 (d, J=18.5 Hz, 1 H), 7.11 (ddd, J=2.8, 7.7, 8.7 Hz, 1 H), 7.26 (dd, J=5.5, 8.7 Hz, 1 H), 7.42 (dd, J=2.8, 7.7 Hz, 1 H).

LC/MS (ESI) m/z 373 (M+H⁺).

### Reference Example 160

### tert-butyl 4-(2-bromo-4-fluorophenyl)piperazine-1-carboxylate

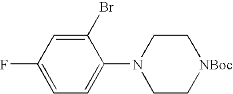

To a solution of tert-butyl 4-(2-bromo-4-fluorophenyl)-3-oxopiperazine-1-carboxylate (10.5 g, 28.0 mmol) in THF (100 mL) was added dropwise 1.0 M borane-THF solution (100 mL, 100 mmol) with stirring at 0° C. This mixture was stirred at room temperature for 30 min. and 1N aqueous sodium hydroxide solution (100 mL) added dropwise at 0° C. This mixture was stirred at room temperature for 1 hr., and the reaction mixture was concentrated under reduced pressure and extracted with ethyl acetate. The organic layer was washed with saturated brine, dried (Na₂SO₄) and concentrated under reduced pressure. The residue was purified by silica gel column chromatography (developing solvent: hexane/ethyl acetate 9/1) to give the title compound as a colorless oil (7.96 g, yield 79%).

¹H NMR (300 MHz, CDCl₃) δ ppm: 1.49 (s, 9 H), 2.90-2.93 (m, 4 H), 3.58-3.61 (m, 4 H), 6.95-7.04 (m, 2 H), 7.33 (ddd, J=0.9, 2.2, 8.2 Hz, 1 H).

LC/MS (ESI) m/z 359 (M+H⁺).

### Reference Example 161

### tert-butyl 4-(4-fluoro-2-formylphenyl)piperazine-1-carboxylate

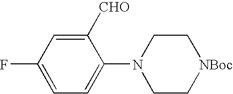

To a solution of tert-butyl 4-(2-bromo-4-fluorophenyl)piperazine-1-carboxylate (7.94 g, 22.1 mmol) in THF (100 mL) was added dropwise.1.6 M n-butyllithium-hexane solution (25.0 mL, 40.0 mmol) with stirring at −78° C. After 1 hr., DMF (3.09 mL, 40.0 mmol) was added dropwise and the mixture was stirred at −78° C. for 10 min. The temperature thereof was raised to room temperature over 1 hr. The reaction mixture was diluted with ethyl acetate, and washed with water and saturated brine. The organic layer was dried (Na₂SO₄) and concentrated under reduced pressure. The residue was purified by silica gel column chromatography (developing solvent: hexane/ethyl acetate=10/1) and crystallized from hexane to give the title compound as a yellow prism crystals (3.53 g, yield 52%).

¹H NMR (300 MHz, CDCl₃) δ ppm: 1.49 (s, 9 H), 2.96-3.00 (m, 4 H), 3.60-3.64 (m, 4 H), 7.13 (dd, J 4.5, 8.9 Hz, 1 H), 7.26 (ddd, J=3.1, 7.5, 8.9 Hz, 1 H), 7.51 (dd, J=3.1, 8.4 Hz, 1 H), 10.38 (d, J=2.8 Hz, 1 H).

LC/MS (ESI) m/z 309 (M+H⁺).

### Reference Example 162

### 1-(4-fluoro-2-formylphenyl)piperazine dihydrochloride

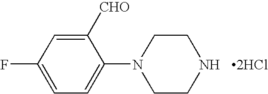

To a solution (75 mL) of tert-butyl 4-(4-fluoro-2-formylphenyl)piperazine-1-carboxylate (3.39 g, 11.0 mmol) in dioxane was added 4N hydrochloric acid-dioxane solution (75 mL) and the mixture was stirred at 60° C. for 4 hrs. After cooling, crystals were collected by filtration, and washed with diethyl ether to give the title compound as a pale brown crystalline powder (2.46 g, yield 80%).

¹H NMR (300 MHz, DMSO-d₆) δ ppm: 3.19-3.33 (m, 8 H), 7.36 (dd, J=4.6, 8.8 Hz, 1 H), 7.47-7.56 (m, 2 H), 9.25 (brs, 2 H), 10.26 (d, J=2.6 Hz, 1 H).

LC/MS (ESI) m/z 209 (M+H⁺) −2HCl.

The compound described in the following Reference Example 163 was produced in the similar manner as in Reference Example 162.

### Reference Example 163

### N-phenyl-1-piperazinecarboxamide hydrochloride

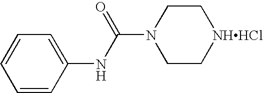

¹H NMR (300 MHz, CDCl₃) δ ppm: 1.48 (s, 9 H), 3.03-3.18 (m, 4 H), 3.60-3.75 (m, 4 H), 6.92-6.98 (m, 1 H), 7.21-7.28 (m, 2 H), 7.44-7.47 (m, 2 H), 8.79 (s, 1 H), 9.19 (brs, 1 H).

### Reference Example 164

### tert-butyl 4-phenylcarbamoyl-1-piperazinecarboxylate

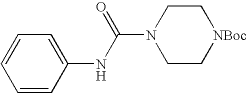

To a solution of tert-butyl piperazine-1-carboxylate (1.86 g, 10.0 mmol) in THF (20 mL) was added dropwise phenyl isocyanate (1.09 mL, 10.0 mmol) with stirring at room temperature. After 30 min., the reaction mixture was concentrated under reduced pressure, and the residue was recrystallized from acetone to give the title compound as colorless prism crystals (2.86 g, yield 94%).

¹H NMR (300 MHz, CDCl₃) δ ppm: 1.48 (s, 9 H), 3.44-3.54 (m, 8 H), 3.65 (brs, 1 H), 7.02-7.08 (m, 1 H), 7.26-7.37 (m, 4 H).

### Reference Example 165

### 2-chloro-N,N-dimethyl-5-nitrobenzamide

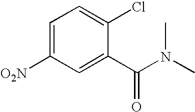

A mixed solution of 2-chloro-5-nitrobenzoic acid (4.03 g), dimethylamine hydrochloride (1.96 g), N,N-diisopropylethylamine (4.25 mL), WSC (5.75 g) and HOBt (3.98 g) in acetonitrile (50 mL) was stirred at room temperature for 12 hrs. The reaction solution was poured into water and stirred for 1 hr. The precipitates were collected by filtration, washed with water and hexane and dried to give the title compound as a pale yellow powder (3.66 g, yield 80%).

¹H NMR (200 MHz, CDCl₃) δ ppm: 2.93 (s, 3 H), 3. 17 (s, 3 H), 7.58-7.63 (m, 1 H), 8.17-8.23 (m, 2 H).

The compounds described in the following Reference Examples 166-179 were produced in the similar manner as in Reference Example 165.

### Reference Example 166

### 4-chloro-N,N-dimethyl-3-nitrobenzamide

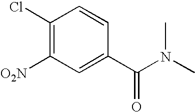

¹H NMR (200 MHz, CDCl₃) δ ppm: 2.95-3.18 (m, 6 H), 7.61 (s, 2 H), 7.96 (s, 1 H).

### Reference Example 167

### 2-fluoro-N,N-dimethyl-5-nitrobenzamide

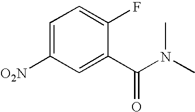

¹H NMR (200 MHz, CDCl₃) δ ppm: 2.96 (d, J 1.5 Hz, 3 H), 3.16 (s, 3 H), 7.22-7.34 (m, 1 H), 8.24-8.38 (m, 2 H).

### Reference Example 168

### 4-fluoro-N,N-dimethyl-3-nitrobenzamide

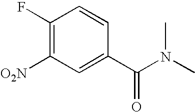

¹H NMR (300 MHz, CDCl₃) δ ppm: 2.98-3.17 (m, 6 H), 7.35 (dd, J=10.5, 8.6 Hz, 1 H), 7.68-7.78 (m, 1 H), 8.15 (dd, J=7.1, 2.2 Hz, 1 H).

### Reference Example 169

### N,N,4-trimethyl-3-nitrobenzamide

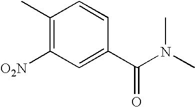

¹H NMR (200 MHz, CDCl₃) δ ppm: 2.64 (s, 3 H), 2.98-3.18 (m, 6 H), 7.40 (d, J=8.2 Hz, 1 H), 7.59 (dd, J=7.8, 2.0 Hz, 1 H), 8.05 (d, J=1.5 Hz, 1 H).

### Reference Example 170

### N,N,2-trimethyl-5-nitrobenzamide

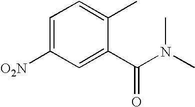

¹H NMR (200 MHz, CDCl₃) δ ppm: 2.41 (s, 3 H), 2.87 (s, 3 H), 3.17 (s, 3 H), 7.40 (dd, J=8.4, 0.7 Hz, 1 H), 8.04-8.20 (m, 2 H).

### Reference Example 171

### N,N-dimethyl-3-nitro-5-(trifluoromethyl)benzamide

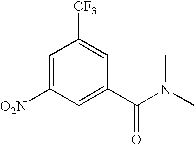

¹H NMR (200 MHz, CDCl₃) δ ppm: 2.98-3.22 (m, 6 H), 8.01-8.08 (m, 1 H), 8.46-8.51 (m, 1 H), 8.52-8.58 (m, 1 H).

### Reference Example 172

### 4-methoxy-N,N-dimethyl-3-nitrobenzamide

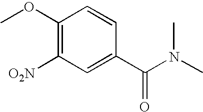

¹H NMR (200 MHz, CDCl₃) δ ppm: 2.23 (s, 6 H), 3.40 (s, 2 H), 3.96 (s, 3 H), 7.04 (d, J 8.4 Hz, 1 H), 7.50 (dd, J=8.4, 2.2 Hz, 1 H), 7.80 (d, J=2.2 Hz, 1 H).

### Reference Example 173

### N,N,2-trimethyl-3-nitrobenzamide

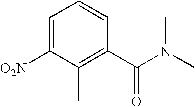

¹H NMR (200 MHz, CDCl₃) δ ppm: 2.46 (s, 3 H), 2.85 (s, 3 H), 3.17 (s, 3 H), 7.34-7.46 (m, 2 H), 7.84-7.93 (m, 1 H).

### Reference Example 174

### 4-ethoxy-N,N-dimethyl-3-nitrobenzamide

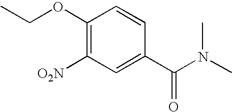

¹H NMR (200 MHz, CDCl₃) δ ppm: 1.49 (t, J=7.1 Hz, 3 H), 3.08 (s, 6 H), 4.23 (q, J=7.0 Hz, 2 H), 7.10 (d, J=8.8 Hz, 1 H), 7.65 (dd, J=8.8, 2.2 Hz, 1 H), 7.93 (d, J=2.2 Hz, 1 H).

### Reference Example 175

### N,N-dimethyl-4-[(1-methylethyl)oxy]-3-nitrobenzamide

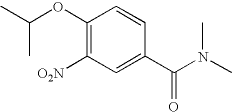

¹H NMR (200 MHz, CDCl₃) δ ppm: 1.42 (d, J=5.9 Hz, 6 H), 3.08 (s, 6 H), 4.64-4.81 (m, 1 H), 7.10 (d, J=8.8 Hz, 1 H), 7.63 (dd, J=8.8, 2.2 Hz, 1 H), 7.89 (d, J=2.2 Hz, 1 H).

### Reference Example 176

### 4-ethyl-N,N-dimethyl-3-nitrobenzamide

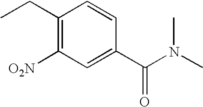

¹H NMR (200 MHz, CDCl₃) δ ppm: 1.30 (t, J=7.5 Hz, 3 H), 2.95 (q, J=7.5 Hz, 2 H), 3.03 (s, 3 H), 3.13 (s, 3 H), 7.43 (d, J=8.1 Hz, 1 H), 7.61 (dd, J=8.1, 1.8 Hz, 1 H), 7.96 (d, J=1.8 Hz, 1 H).

### Reference Example 177

### N,N-dimethyl-3-nitro-4-trifluoromethoxybenzamide

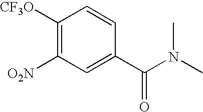

¹H NMR (300 MHz, CDCl₃) δ ppm: 3.04 (s, 3 H), 3.14 (s, 3 H), 7.43-7.59 (m, 1 H), 7.74 (dd, J=8.5, 2.1 Hz, 1 H), 8.06 (d, J=2.1 Hz, 1 H).

### Reference Example 178

### N,N-dimethyl-3-nitro-4-propoxybenzamide

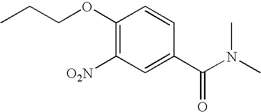

¹H NMR (300 MHz, CDCl₃) δ ppm: 1.07 (t, J=7.5 Hz, 3 H), 1.78-1.95 (m, 2 H), 3.07 (s, 6 H), 4.10 (t, J=6.4 Hz, 2 H), 7.09 (d, J=8.6 Hz, 1 H), 7.64 (dd, J=8.6, 2.2 Hz, 1 H), 7.93 (d, J=2.2 Hz, 1 H).

### Reference Example 179

### N,N-dimethyl-3-nitro-4-(trifluoromethyl)benzamide

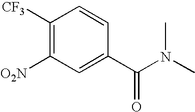

¹H NMR (300 MHz, CDCl₃) δ ppm: 3.01 (s, 3 H), 3.15 (s, 3 H), 7.71-7.81 (m, 1 H), 7.90 (d, J=8.1 Hz, 1 H), 7.94 (d, J=0.6 Hz, 1 H).

### Reference Example 180

### (2-chloro-5-nitrobenzyl)dimethylamine

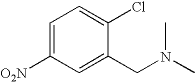

To 1.0 M borane-THF solution (55 mL) was added 2-chloro-N,N-dimethyl-5-nitrobenzamide (3.66 g). The reaction solution was heated under reflux for 6 hrs. and water was added to the reaction solution under ice-cooling, and the mixture was concentrated. The residue was dissolved in methanol (10 mL) and 6N hydrochloric acid (30 mL) was added. The mixture was heated under reflux for 16 hrs. After cooling, the reaction solution was basified with aqueous sodium hydroxide and extracted with tetrahydrofuran/ethyl acetate=1/1. The organic layer was dried (MgSO₄) and concentrated. The residue was purified by silica gel column chromatography (developing solvent: hexane-hexane/ethyl acetate=5/1) to give the title compound (2.90 g, yield 84%) as a yellow oil.

¹H NMR (200 MHz, CDCl₃) δ ppm: 2.33 (s, 6 H), 3.59. (s, 2 H), 7.52 (d, J=8.8 Hz, 1 H), 8.06 (dd, J=8.6, 2.8 Hz, 1 H), 8.38 (d, J=2.9 Hz, 1 H).

The compounds described in the following Reference Examples 181-194 were produced in the similar manner as in Reference Example 180.

### Reference Example 181

### (4-chloro-3-nitrobenzyl)dimethylamine

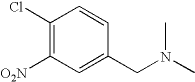

¹H NMR (200 MHz, CDCl₃) δ ppm: 2.25 (s, 6 H), 3.45 (s, 2 H), 7.49 (d, J=1.1 Hz, 2 H), 7.85 (s, 1 H).

### Reference Example 182

### (2-fluoro-5-nitrobenzyl)dimethylamine

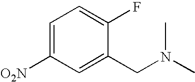

¹H NMR (200 MHz, CDCl₃) δ ppm: 2.30 (s, 6 H), 3.55 (s, 2 H), 7.18 (t, J=8.8 Hz, 1 H), 8.10-8.22 (m, 1 H), 8.35 (dd, J=6.2, 2.9 Hz, 1 H).

### Reference Example 183

### (4-fluoro-3-nitrobenzyl)dimethylamine

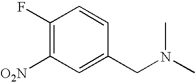

¹H NMR (200 MHz, CDCl₃) δ ppm: 2.25 (s, 6 H), 3.45 (s, 2 H), 7.17-7.30 (m, 1 H), 7.54-7.67 (m, 1 H), 8.02 (dd, J=7.3, 2.2 Hz, 1 H).

### Reference Example 184

### dimethyl(2-methyl-5-nitrobenzyl)amine

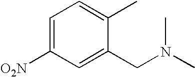

¹H NMR (200 MHz, CDCl₃) δ ppm:2.26 (s, 6 H), 2.45 (s, 3 H), 3.43 (s, 2 H), 7.29 (d, J=8.4 Hz, 1 H), 8.02 (dd, J=8.2, 2.4 Hz, 1 H), 8.17 (d, J =2.2 Hz, 1 H).

### Reference Example 185

### dimethyl(4-methyl-3-nitrobenzyl)amine

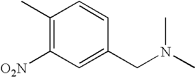

¹H NMR (200 MHz, CDCl₃) δ ppm: 2.25 (s, 6 H), 2.58 (s, 3 H), 3.45 (s, 2 H), 7.25-7.34 (m, 1 H), 7.47 (dd, J 7.9, 1.7 Hz, 1 H), 7.92 (d, J=1.8 Hz, 1 H).

### Reference Example 186

### dimethyl[3-nitro-5-(trifluoromethyl)benzyl]amine

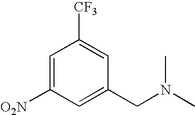

¹H NMR (200 MHz, CDCl₃) δ ppm: 2.28 (s, 6 H), 3.57 (s, 2 H), 7.95 (s, 1 H), 8.38 (s, 2 H).

### Reference Example 187

### (4-methoxy-3-nitrobenzyl)dimethylamine

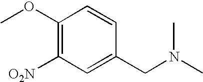

¹H NMR (200 MHz, CDCl₃) δ ppm: 2.23 (s, 6 H), 3.40 (s, 2 H), 3.96 (s, 3 H), 7.04 (d, J=8.4 Hz, 1 H), 7.50 (dd, J=8.4, 2.2 Hz, 1 H), 7.80 (d, J 2.2 Hz, 1 H).

### Reference Example 188

### dimethyl(2-methyl-3-nitrobenzyl)amine

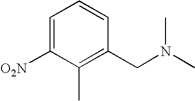

¹H NMR (300 MHz, CDCl₃) δ ppm: 2.24 (s, 6 H), 2.47 (s, 3 H), 3.43 (s, 2 H), 7.21-7.29 (m, 1 H), 7.47 (dd, J=7.7, 0.9 Hz, 1 H), 7.65 (dd, J=8.1, 1.2 Hz, 1 H).

### Reference Example 189

### (4-ethyl-3-nitrobenzyl)dimethylamine

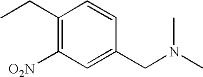

¹H NMR (200 MHz, CDCl₃) δ ppm: 1.28 (t, J=7.5 Hz, 3 H), 2.25 (s, 6 H), 2.90 (q, J=7.3 Hz, 2 H), 3.44 (s, 2 H), 7.31 (d, J=7.7 Hz, 1 H), 7.48 (dd, J=7.7, 1.8 Hz, 1 H), 7.82 (d, J=1.8 Hz, 1 H).

### Reference Example 190

### (4-ethoxy-3-nitrobenzyl)dimethylamine

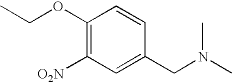

¹H NMR (300 MHz, CDCl₃) δ ppm: 1.46 (t, J=6.7 Hz, 3 H), 2.23 (s, 6 H), 3.38 (s, 2 H), 4.17 (q, J=7.0 Hz, 2 H), 7.01 (d, J=8.6 Hz, 1 H), 7.45 (dd, J=8.6, 2.2 Hz, 1 H), 7.74 (d, J=2.2 Hz, 1 H).

### Reference Example 191

### (4-isopropoxy-3-nitrobenzyl)dimethylamine

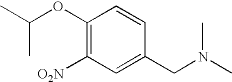

¹H NMR (300 MHz, CDCl₃) δ ppm: 1.39 (d, J=6.1 Hz, 6 H), 2.23 (s, 6 H), 3.37 (s, 2 H), 4.57-4.71 (m, 1 H), 7.01 (d, J=8.6 Hz, 1 H), 7.43 (dd, J=8.6, 2.2 Hz, 1 H), 7.70 (d, J=2.2 Hz, 1 H).

### Reference Example 192

### dimethyl[3-nitro-4-(trifluoromethoxy)benzyl]amine

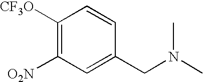

¹H NMR (300 MHz, CDCl₃) δ ppm: 2.26 (s, 6 H), 3.48 (s, 2 H), 7.39 (dd, J=8.5, 1.3 Hz, 1 H), 7.62 (dd, J=8.4, 2.2 Hz, 1 H), 7.96 (d, J=2.1 Hz, 1 H).

### Reference Example 193

### (4-propoxy-3-nitrobenzyl)dimethylamine

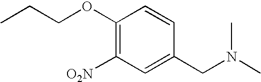

¹H NMR (300 MHz, CDCl₃) δ ppm: 1.06 (t, J =7.4 Hz, 3 H), 1.77-1.93 (m, 2 H), 2.23 (s, 6 H), 3.38 (s, 2 H), 4.06 (t, J=6.4 Hz, 2 H), 7.01 (d, J=8.5 Hz, 1 H), 7.46 (dd, J=8.6, 2.2 Hz, 1 H), 7.76 (d, J=2.1 Hz, 1 H).

### Reference Example 194

### dimethyl[3-nitro-4-(trifluoromethyl)benzyl]amine

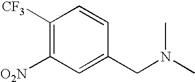

¹H NMR (300 MHz, CDCl₃) δ ppm: 2.27 (s, 6 H), 3.54 (s, 2 H), 7.67 (d, J=5.1 Hz, 1 H), 7.77 (d, J=5.1 Hz, 1 H), 7.88 (s, 1 H).

### Reference Example 195

### (5-amino-2-chlorobenzyl)dimethylamine

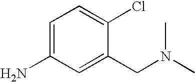

A mixture of (2-chloro-5-nitrobenzyl)dimethylamine (2.90 g), iron (reduced) (3.63 g) and calcium chloride (144 mg) in 80% ethanol was heated under reflux for 4 hrs. After cooling, insoluble materials were removed by celite filtration, and the filtrate was concentrated. The obtained residue was recrystallized from diisopropyl ether-hexane to give the title compound (1.47 g, yield 59%).

¹H NMR (200 MHz, CDCl₃) δ ppm: 2.29 (s, 6 H), 3.45 (s, 2 H), 3.63 (s, 2 H), 6.52 (dd, J=8.4, 2.9 Hz, 1 H), 6.78 (d, J=2.9 Hz, 1 H), 7.10 (d, J=8.4 Hz, 1 H).

The compounds described in the following Reference Examples 196-199 were produced in the similar manner as in Reference Example 195.

### Reference Example 196

### (5-amino-2-fluorobenzyl)dimethylamine

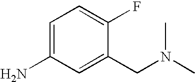

¹H NMR (300 MHz, CDCl₃) δ ppm: 2.26 (s, 6 H), 3.40 (d, J=1.2 Hz, 2 H), 3.52 (s, 2 H), 6.47-6.58 (m, 1 H), 6.66 (dd, J=6.1, 2.9 Hz, 1 H), 6.76-6.87 (m, 1 H).

### Reference Example 197

### (3-amino-4-methylbenzyl)dimethylamine

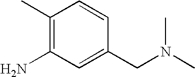

¹H NMR (300 MHz, CDCl₃) δ ppm: 2.14 (s, 3 H), 2.22 (s, 6 H), 3.31 (s, 2 H), 3.56 (s, 2 H), 6.57-6.68 (m, 2 H), 6.97 (d, J=7.6 Hz, 1 H).

### Reference Example 198

### [3-amino-4-(trifluoromethoxy)benzyl]dimethylamine

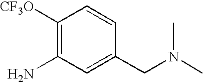

¹H NMR (300 MHz, CDCl₃) δ ppm: 2.23 (s, 6 H), 3.31 (s, 2 H), 3.83 (s, 2 H), 6.65 (dd, J=8.3, 1.9 Hz, 1 H), 6.79 (d, J=1.9 Hz, 1 H), 7.06 (dd, J=8.3, 1.5 Hz, 1 H).

### Reference Example 199

### (3-amino-2-methylbenzyl)dimethylamine

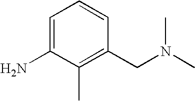

¹H NMR (300 MHz, CDCl₃) δ ppm: 2.17 (s, 3 H), 2.22 (s, 6 H), 3.35 (s, 2 H), 3.60 (s, 2 H), 6.63 (d, J=7.7 Hz, 1 H), 6.68 (d, J=7.0 Hz, 1 H), 6.96 (t, J=7.6 Hz, 1 H).

### Reference Example 200

### (5-amino-2-methylbenzyl) dimethylamine dihydrochloride

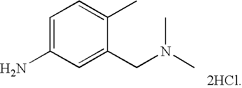

A mixture of dimethyl(2-methyl-5-nitrobenzyl)amine (1.17 g), iron (reduced) (1.68 g) and calcium chloride (67 mg) in 80% ethanol (30 mL) was heated under reflux for 4 hrs. After cooling, insoluble materials were removed by celite filtration, and the filtrate was concentrated. The obtained residue was purified by aminopropyl silica gel chromatography (developing solvent: hexane/ethyl acetate=5/1-1/1) to give an orange oil. The obtained residue was dissolved in ethyl acetate (15 mL), and 4N hydrochloric acid-ethyl acetate solution (5 mL) was added. The obtained precipitate was washed with ethanol-ethyl acetate and dried to give the title compound (1.99 g, yield 99%).

¹H NMR (200 MHz, CDCl₃) δ ppm: 2.22 (s, 3 H), 2.25 (s, 6 H), 3.30 (s, 2 H), 3.51 (s, 2 H), 6.52 (dd, J=8.1, 2.6 Hz, 1 H), 6.68 (d, J=2.6 Hz, 1 H), 6.93 (d, J=8.1 Hz, 1 H).

The compounds described in the following Reference Examples 201-204 were produced in the similar manner as in Reference Example 200.

### Reference Example 201

### (3-amino-4-chlorobenzyl)dimethylamine dihydrochloride

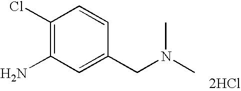

¹H NMR (300 MHz, DMSO-d₆) δ ppm: 2.66 (d, J=4.9 Hz, 6 H), 4.14 (d, J=5.6 Hz, 2 H), 6.13 (s, 3 H), 6.91 (dd, J=8.3, 2.0 Hz, 1 H), 7.00 (d, J=2.0 Hz, 1 H), 7.30 (d, J=8.1 Hz, 1 H), 10.92 (.s, 1 H).

### Reference Example 202

### (3-amino-4-fluorobenzyl)dimethylamine dihydrochloride

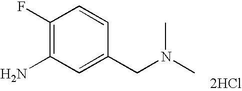

¹H NMR (300 MHz, DMSO-d₆) δ ppm: 2.66 (d, J=4.6 Hz, 6 H), 4.21 (d, J=5.1 Hz, 2 H), 7.20-7.38 (m, 3 H), 8.10 (s, 3 H), 11.08 (s, 1 H).

### Reference Example 203

### [3-amino-5-(trifluoromethyl)benzyl]dimethylamine dihydrochloride

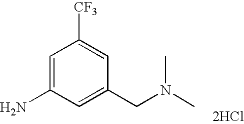

¹H NMR (300 MHz, DMSO-d₆) δ ppm: 2.69 (d, J=4.9 Hz, 6 H), 4.25 (d, J=5.4 Hz, 2 H), 5.60 (s, 3 H), 7.14 (s, 2 H), 7.32 (s, 1 H), 10.83 (s, 1 H).

### Reference Example 204

### {[3-amino-4-(methyloxy)phenyl]methyl}dimethylamine dihydrochloride

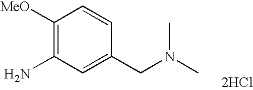

¹H NMR (300 MHz, DMSO-d₆) δ ppm: 2.64 (d, J=3.7 Hz, 6 H), 3.88 (s, 3 H), 4.19 (d, J=3.9 Hz, 2 H), 7.19 (d, J=8.3 Hz, 1 H), 7.38-7.51 (m, 2 H).

### Reference Example 205

### (3-amino-4-ethylbenzyl)dimethylamine dihydrochloride

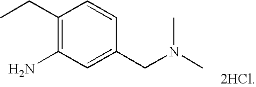

To a suspension of (4-ethyl-3-nitrobenzyl)dimethylamine (1.60 g) and 10% palladium-carbon (0.25 g) in ethanol (20 mL) was slowly added hydrazine monohydrate (1.02 mL) at room temperature and the mixture was stirred for 1 hr. Palladium was removed by filtration and the filtrate was concentrated to give a pale yellow oil. The obtained residue was dissolved in ethyl acetate (15 mL) and 4N hydrochloric acid-ethyl acetate solution (5 mL) was added. The obtained precipitate was washed with ethanol-ethyl acetate and dried to give the title compound (0.94 g, yield 49%).

¹H NMR (300 MHz, DMSO-d₆) δ ppm: 1.21 (t, J=7.5 Hz, 3 H), 2.68 (d, J=2.7 Hz, 6 H), 2.74 (q, J=7.6 Hz, 2 H), 4.26 (d, J=3.7 Hz, 2 H), 7.38 (d, J=8.1 Hz, 1 H), 7.46 (s, 1 H), 7.52 (dd, J=8.1, 1.0 Hz, 1 H), 10.98 (s, 1 H).

The compounds described in the following Reference Examples 206-209 were produced in the similar manner as in Reference Example 205.

### Reference Example 206

### (3-amino-4-ethoxybenzyl)dimethylamine dihydrochloride

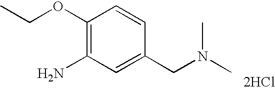

¹H NMR (300 MHz, DMSO-d₆) δ ppm: 1.39 (t, J=7.0 Hz, 3 H), 2.65 (d, J=2.9 Hz, 6 H), 4.17 (q, J=6.9 Hz, 2 H), 4.21 (s, 2 H), 7.20 (d, J=8.3 Hz, 1 H), 7.43-7.54 (m, 2 H), 10.97 (s, 1 H).

### Reference Example 207

### (3-amino-4-isopropoxybenzyl)dimethylamine dihydrochloride

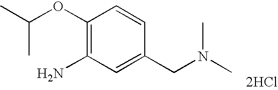

¹H NMR (300 MHz, DMSO-d₆) δ ppm: 1.33 (d, J=5.9 Hz, 6 H), 2.66 (d, J=3.7 Hz, 6 H), 4.20 (d, J=4.2 Hz, 2 H), 4.68-4.83 (m, 1 H), 7.22 (d, J=9.3 Hz, 1 H), 7.44-7.54 (m, 2 H), 10.96 (s, 1 H).

### Reference Example 208

### 4-[(dimethylamino)methyl]-N¹,N¹-dimethylbenzene-1,2-diamine trihydrochloride

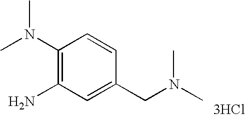

¹H NMR (300 MHz, DMSO-d₆) δ ppm: 2.68 (d, J=4.7 Hz, 6 H), 3.03 (s, 6 H), 4.18 (d, J=5.5 Hz, 2 H), 7.03 (d, J=1.5 Hz, 1 H), 7.11 (dd, J=8.5, 1.5 Hz, 1 H), 7.58 (d, J=8.5 Hz, 1 H), 10.98 (s, 1 H).

### Reference Example 209

### (3-amino-4-pyrrolidin-1-ylbenzyl)dimethylamine trihydrochloride

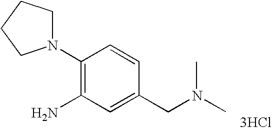

¹H NMR (300 MHz, DMSO-d₆) δ ppm: 2.03-2.16 (m, 4 H), 2.67 (d, J=4.9 Hz, 6 H), 3.54 (s, 4 H), 4.17 (d, J=5.3 Hz, 2 H), 7.05-7.25 (m, 2 H), 7.37 (d, J=8.3 Hz, 1 H), 10.95 (s, 1 H).

### Reference Example 210

### (3-amino-4-propoxybenzyl)dimethylamine

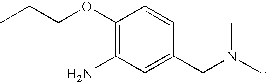

To a suspension of (4-propoxy-3-nitrobenzyl)dimethylamine (1.13 g) and 10% palladium-carbon (0.15 g) in ethanol (20 mL) was slowly added hydrazine monohydrate (0.728 mL) at room temperature, and the mixture was stirred for 2 hrs. Palladium was removed by filtration, and the filtrate was concentrated to give the title compound (0.54 g, yield 55%).

¹H NMR (300 MHz, CDCl₃) δ ppm: 1.05 (t, J=7.4 Hz, 3 H), 1.74-1.93 (m, 2 H), 2.21 (s, 6 H), 3.77 (s, 2 H), 3.93 (q, J=6.3 Hz, 2 H), 6.47-6.64 (m, 2 H), 6.64-6.74 (m, 2 H).

The compound described in the following Reference Example 211 was produced in the similar manner as in Reference Example 210.

### Reference Example 211

### 5-[(dimethylamino)methyl]-2-(trifluoromethyl)aniline

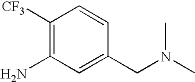

¹H NMR (300 MHz, CDCl₃) δ ppm: 2.24 (s, 6 H), 3.35 (s, 2 H), 4.13 (s, 2 H), 6.63-6.77 (m, 2 H), 7.36 (d, J=7.9 Hz, 1 H).

### Reference Example 212

### (3-aminobenzyl)diethylamine dihydrochloride

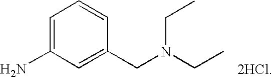

A mixture of 3-nitrobenzylchloride (5.0 g), diethylamine (9.0 mL) in THF (50 ml) was stirred at 60° C. for 2 hrs., and the reaction mixture was poured into water and extracted with ethyl acetate. The organic layer was washed with 1N hydrochloric acid. The aqueous layer was basified with 2N aqueous sodium hydroxide, and extracted with ethyl acetate and dried (MgSO₄). The solvent was evaporated to give an orange oil. To a suspension of the obtained residue and 10% palladium-carbon (0.5 g) in ethanol (50 mL) was slowly added hydrazine monohydrate (2.91 mL) at room temperature, and the mixture was stirred for 1 hr. Palladium was removed by filtration, and the filtrate was concentrated. The obtained residue was purified by silica gel column chromatography (developing solvent: hexane/ethyl acetate=10/1−ethyl acetate) to give a pale yellow oil. The obtained residue was dissolved in ethyl acetate (50 mL), and 4N hydrochloric acid-ethyl acetate solution (20 mL) was added. The obtained precipitate was washed with methanol-ethyl acetate and dried to give the title compound as a white powder (4.01 g, yield 55%).

¹H NMR (300 MHz, DMSO-d₆) δ ppm: 1.19-1.32 (m, 6 H), 2.92-3.12 (m, 4 H), 4.32 (d, J 4.3 Hz, 2 H), 7.41 (d, J=8.4 Hz, 1 H), 7.46-7.63 (m, 2 H), 7.68 (d, J=7.7 Hz, 1 H), 11.03 (s, 1 H)

### Reference Example 213

### methyl 4-(2-methoxyethoxy)-3-nitrobenzoate

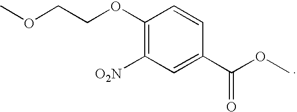

To a suspension of sodium hydride (60% oil, 1.80 g) in DMF (45 mL) was added methyl 4-(2-hydroxyethoxy)benzoate (7.85 g) at room temperature and the mixture was stirred for 15 min. Methyl iodide (6.25 g) was added to the reaction mixture and the mixture was stirred at 80° C. for 2 hrs. After cooling, the reaction mixture was poured into water and extracted with ethyl acetate. The organic layer was washed with water and saturated brine and dried (MgSO₄). The solvent was evaporated and the obtained residue was purified by silica gel column chromatography (developing solvent: hexane/ethyl acetate=9/1-1/1-1/4) to give a white powder (6.81 g, yield 81%).

To a solution of the obtained powder in acetic anhydride (40 mL) was added concentrated nitric acid (6.0 mL) at 0° C. and the mixture was stirred at room temperature for 16 hrs. The reaction mixture was poured into ice water and the mixture was extracted with ethyl acetate. The organic layer was dried (MgSO₄) and the solvent was evaporated. The obtained residue was purified by silica gel column chromatography (developing solvent: hexane/ethyl acetate=20/1-1/1) to give the title compound as a pale yellow powder (5.23 g, yield 50%).

¹H NMR (300 MHz, CDCl₃) δ ppm: 3.46 (s, 3 H), 3.76-3.86 (m, 2 H), 3.93 (s, 3 H), 4.24-4.39 (m, 2 H), 7.16 (d, J=8.9 Hz, 1 H), 8.20 (dd, J=8.9, 2.1 Hz, 1 H), 8.51 (d, J=2.3 Hz, 1 H).

### Reference Example 214

### 4-(chloromethyl)-1-(2-methoxyethoxy)-2-nitrobenzene

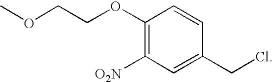

To a solution of methyl 4-(2-methoxyethoxy)-3-nitrobenzoate (5.23 g) in THF (200 mL) was added lithium borohydride (2.18 g) at 0° C. and the mixture was stirred at room temperature for 16 hrs. To the reaction mixture was added 1N hydrochloric acid and the mixture was extracted with ethyl acetate. The organic layer was dried (MgSO₄) and the solvent was evaporated to give a pale yellow oil. The obtained residue was slowly added to thionyl chloride (20 mL) and the mixture was stirred at room temperature for 2 hrs. Excess thionyl chloride was evaporated and the residue was extracted with saturated aqueous solution of sodium hydrogen carbonate-ethyl acetate. The organic layer was dried (MgSO₄) and the solvent was evaporated. The residue was purified by silica gel column chromatography (developing solvent: hexane/ethyl acetate=10/3-1/1) to give the title compound as a pale yellow powder (3.68 g, yield 86%).

¹H NMR (300 MHz, CDCl₃) δ ppm: 3.46 (s, 3 H), 3.72-3.84 (m, 2 H), 4.20-4.33 (m, 2 H), 4.57 (s, 2 H), 7.12 (d, J=8.5 Hz, 1 H), 7.55 (dd, J=8.7, 2.5 Hz, 1 H), 7.88 (d, J=2.3 Hz, 1 H).

### Reference Example 215

### 1-[3-amino-4-(2-methoxyethoxy)phenyl]-N,N-dimethylmethanamine dihydrochloride

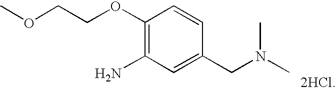

A solution of 4-(chloromethyl)-1-(2-methoxyethoxy)-2-nitrobenzene (3.68 g) in THF (5 mL) was added to 50% dimethyl amine solution (25 mL) and the mixture was stirred at room temperature for 3 days. The reaction solution was poured into saturated aqueous solution of sodium hydrogen carbonate and the mixture was extracted with ethyl acetate. The organic layer was dried (MgSO₄) and the solvent was evaporated. To a suspension of the obtained residue and 10% palladium-carbon (0.5 g) in ethanol (45 mL) was slowly added hydrazine monohydrate (1.43 mL) at room temperature and the mixture was stirred for 2 hrs. Palladium was removed by filtration, and the filtrate was concentrated. The residue was purified by aminopropyl silica gel column chromatography (developing solvent: hexane/ethyl acetate=20/1-1/1) to give a pale yellow oil. The obtained residue was dissolved in ethyl acetate (30 mL), and 4N hydrochloric acid-ethyl acetate solution (10 mL) was added. The obtained precipitate was washed with ethanol-ethyl acetate and dried to give the title compound (3.07 g, yield 69%).

¹H NMR (300 MHz, DMSO-d₆) δ ppm: 2.67 (d, J=4.5 Hz, 6 H), 3.34 (s, 3 H), 3.66-3.83 (m, 2 H), 4.09-4.30 (m, 4 H), 7.10-7.24 (m, 1-H), 7.29-7.45 (m, 2 H), 10.70 (s, 1 H).

### Reference Example 216

### [3-nitro-4-(trifluoromethoxy)phenyl]methanol

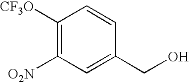

3-Nitro-(4-trifluoromethoxy)benzoic acid (4.65 g) was added to 1.0 M borane-THF solution (50 mL) at room temperature and the mixture was stirred for 16 hrs. Water was added to the reaction mixture until production of hydrogen was ceased and then concentrated. To the residue were added saturated aqueous solution of sodium hydrogen carbonate and ethyl acetate and the mixture was subjected to extraction. The organic layer was washed with saturated brine, dried (MgSO₄) and the solvent was evaporated. The residue was purified by silica gel column chromatography (developing solvent: hexane/ethyl acetate=4/1-1/1) to give the title compound (3.76 g, yield 86%).

1H NMR (300 MHz, CDCl₃) δ ppm: 1.93 (t, J=5.5 Hz, 1 H), 4.81 (d, J=5.1 Hz, 2 H), 7.44 (dd, J=8.4, 1.3 Hz, 1 H), 7.60-7.70 (m, 1 H), 8.00 (d, J=2.0 Hz, 1 H).

### Reference Example 217

### 4-(chloromethyl)-2-nitro-1-(trifluoromethoxy)benzene

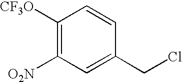

[3-Nitro-4-(trifluoromethoxy)phenyl]methanol (3.76 g) was slowly added to thionyl chloride (50 mL) and the mixture was stirred at 50° C. for 3 hrs. Excess thionyl chloride was evaporated and the residue was extracted with saturated aqueous solution of sodium hydrogen carbonate-ethyl acetate. The organic layer was dried (MgSO₄) and the solvent was evaporated. The residue was purified by silica gel column chromatography (developing solvent: hexane/ethyl acetate 10/1-2/1) to give the title compound as a yellow oil (3.64 g, yield 90%).

¹H NMR (300 MHz, CDCl₃) δ ppm: 4.62 (s, 2 H), 7.39-7.49 (m, 1 H), 7.68 (dd, J=8.6, 2.2 Hz, 1 H), 8.02 (d, J=2.4 Hz, 1 H)

### Reference Example 218

### {4-[(dimethylamino)methyl]-2-nitrophenyl}dimethylamine

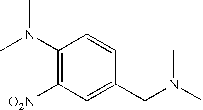

A solution of 4-(chloromethyl)-2-nitro-1-(trifluoromethoxy)benzene (3.64 g) in THF (5 mL) was added to 50% dimethyl amine solution (25 mL) and the mixture was stirred at room temperature for 16 hrs. The reaction solution was poured into saturated aqueous solution of sodium hydrogen carbonate and the mixture was extracted with ethyl acetate. The organic layer was dried (MgSO₄) and the solvent was evaporated. The obtained residue was purified by aminopropyl silica gel column chromatography (developing solvent: hexane/ethyl acetate=4/1-1/4) to give the title compound as an orange oil (2.46 g, yield 78%).

¹H NMR (300 MHz, CDCl₃) δ ppm: 2.23 (s, 6 H), 2.88 (s, 6 H), 3.35 (s, 2 H), 6.99 (d, J=8.7 Hz, 1 H), 7.37 (dd, J=8.7, 2.1 Hz, 1 H), 7.70 (d, J=2.1 Hz, 1 H).

### Reference Example 219

### N,N-dimethyl-1-(3-nitro-4-pyrrolidin-1-ylphenyl)methanamine

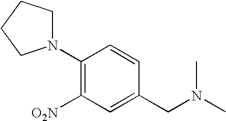

A mixture of dimethyl[3-nitro-4-(trifluoromethoxy)benzyl]amine (2.86 g), pyrrolidine (1.67 mL), N,N-diisopropylethylamine (3.48 mL) and DMF (25 mL) was stirred at 80° C. for 16 hrs. The reaction solution was poured into saturated aqueous solution of sodium hydrogen carbonate and the mixture was extracted with ethyl acetate. The organic layer was dried (MgSO₄) and the solvent was evaporated. The obtained residue was purified by aminopropyl silica gel column chromatography (developing solvent: hexane/ethyl acetate=95/5-2/1) to give the title compound as a yellow oil (1.06 g, yield 39%).

¹H NMR (300 MHz, CDCl₃) δ ppm: 1.91-2.02 (m, 4 H), 2.23 (s, 6 H), 3.16-3.26 (m, 4 H), 3.34 (s, 2 H), 6.87 (d, J=8.9 Hz, 1 H), 7.34 (dd, J=8.7, 2.1 Hz, 1 H), 7.66 (d, J=2.1 Hz, 1 H).

### Reference Example 220

### 4-[(1-methylethyl)oxyl-3-nitrobenzoic acid

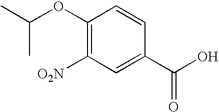

To a solution of 4-[(1-methylethyl)oxy]benzoic acid (5.41 g) in acetic anhydride (35 mL) was added concentrated nitric acid (5.0 mL) at 0° C. and the mixture was stirred at room temperature for 16 hrs. The reaction mixture was poured into ice water. The precipitates were collected by filtration and dried to give the title compound as a pale yellow powder (5.51 g, yield 82%).

¹H NMR (200 MHz, CDCl₃) δ ppm: 1.45 (d, J=5.9 Hz, 6 H), 4.71-4.91 (m, 1 H), 7.14 (d, J=9.2 Hz, 1 H), 8.23 (dd, J=8.8, 2.2 Hz, 1 H), 8.51 (d, J=1.8 Hz, 1 H).

The compound described in the following Reference Example 221 was produced in the similar manner as in Reference Example 220.

### Reference Example 221

### 4-ethoxy-3-nitrobenzoic acid

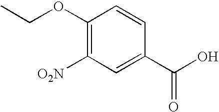

¹H NMR (200 MHz, CDCl₃) δ ppm: 1.52 (t, J=7.0 Hz, 3 H), 4.28 (q, J=7.0 Hz, 2 H), 7.14 (d, J=8.8 Hz, 1 H), 8.25 (dd, J=9.0, 2.0 Hz, 1 H), 8.55 (d, J=2.2 Hz, 1 H).

### Reference Example 222

### 4-ethyl-3-nitrobenzoic acid

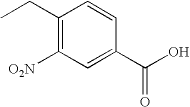

To a solution (8 mL) of 4-ethylbenzoic acid (1.50 g) in concentrated sulfuric acid was slowly added concentrated nitric acid (4 mL) at 0° C. and the mixture was stirred for 1.5 hrs. The reaction mixture was poured into ice water. The precipitates were collected by filtration and dried to give the title compound as a pale yellow powder (1.87 g, yield 96%).

¹H NMR (300 MHz, CDCl₃) δ ppm: 1.33 (t, J=7.5 Hz, 3 H), 3.00 (q, J=7.4 Hz, 2 H), 7.51 (d, J=8.1 Hz, 1 H), 8.23 (dd, J=8.1, 1.7 Hz, 1 H), 8.59 (d, J=1.2 Hz, 1 H).

The compounds described in the following Reference Examples 223-224 were produced in the similar manner as in Reference Example 222.

### Reference Example 223

### 3-nitro-4-trifluoromethoxybenzoic acid

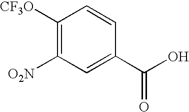

¹H NMR (300 MHz, CDCl₃) δ ppm: 7.56-7.63 (m, 1 H), 8.39 (dd, J=8.7, 2.3 Hz, 1 H), 8.70 (d, J 2.3 Hz, 1 H).

### Reference Example 224

### 3-nitro-4-(propyloxy)benzoic acid

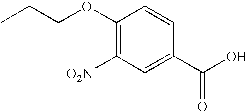

¹H NMR (300 MHz, CDCl₃) δ ppm: 1.09 (t, J=7.4 Hz, 3 H), 1.81-1.99 (m, 2 H), 4.16 (t, J=6.4 Hz, 2 H), 7.14 (d, J=8.9 Hz, 1 H), 8.25 (dd, J=8.9, 2.1 Hz, 1 H), 8.56 (d, J=2.3 Hz, 1 H).

### Reference Example 225

### N-methyl-1-(3-nitrophenyl)methanamine

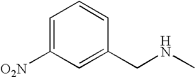

A mixture of 3-nitrobenzaldehyde (1.50 g), methylamine hydrochloride (1.35 g), tetraisopropoxytitanium (5.90 mL) and triethylamine (2.79 mL) in ethanol (15 mL) was stirred at room temperature for 12 hrs. To the reaction mixture was added sodium borohydride (0.57 g) and the mixture was stirred at room temperature for 12 hrs. To the reaction mixture was added 2M aqueous ammonia. The resulting inorganic salt was removed by filtration and washed with dichloromethane. The organic layer was separated and the aqueous layer was extracted with dichloromethane. The organic layers were combined and washed with 1N hydrochloric acid. The aqueous layer was basified with 2N aqueous sodium hydroxide, extracted with dichloromethane and dried (MgSO₄). The solvent was evaporated to give the title compound (1.11 g, yield 67%).

¹H NMR (300 MHz, CDCl₃) δ ppm: 2.47 (s, 3 H), 3.86 (s, 2 H), 7.49 (t, J=7.8 Hz, 1 H), 7.62-7.71 (m, 1 H), 8.06-8.15 (m, 1 H), 8.20 (t, J=1.7 Hz, 1 H).

The compound described in the following Reference Example 226 was produced in the similar manner as in Reference Example 225.

### Reference Example 226

### N-[(3-nitrophenyl)methyl]cyclopropanamine

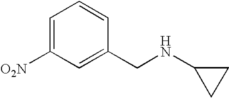

¹H NMR (200 MHz, CDCl₃) δ ppm: 0.33-0.50 (m, 4 H), 2.08-2.22 (m, 1 H), 3.95 (s, 2 H), 7.48 (t, J=7.9 Hz, 1 H), 7.66 (d, J=7.7 Hz, 1 H), 8.05-8.15 (m, 1 H), 8.17-8.22 (m, 1 H).

### Reference Example 227

### tert-butyl (3-aminobenzyl)methylcarbamate

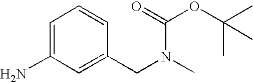

A mixture of N-methyl-1-(3-nitrophenyl)methanamine (1.11 g) and di-tert-butyl dicarbonate (1.53 g) in acetonitrile (15 mL) was stirred at room temperature for 12 hrs. The solvent was evaporated and the residue was purified by silica gel column chromatography (developing solvent: hexane/ethyl acetate=10/1-5/1) to give a pale yellow oil. To a suspension of the obtained residue and 10% palladium-carbon (0.2 g) in ethanol (15 mL) was slowly added hydrazine monohydrate (1.07 mL) at room temperature, and the mixture was stirred for 30 min. Palladium was removed by filtration, and the filtrate was concentrated to give the title compound as a pale yellow oil (1.58 g, yield 100%).

¹H NMR (300 MHz, CDCl₃) δ ppm: 1.48 (s, 9 H), 2.81 (s, 3 H), 3.65 (s, 2 H), 4.33 (s, 2 H), 6.38-6.71 (m, 3 H), 7.09 (t, J=7.8 Hz, 1 H).

The compound described in the following Reference Example 228 was produced in the similar manner as in Reference Example 227.

### Reference Example 228

### tert-butyl (3-aminobenzyl)cyclopropylcarbamate

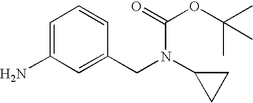

¹H NMR (300 MHz, CDCl₃) δ ppm: 0.58-0.77 (m, 4 H), 1.46 (s, 9 H), 2.46 (s, 1 H), 3.63 (s, 2 H), 4.33 (s, 2 H), 6.56 (dd, J=4.5, 1.6 Hz, 2 H), 6.62 (d, J=7.6 Hz, 1 H), 7.08 (dd, J=8.4, 7.7 Hz, 1 H).

### Reference Example 229

### 2-(trifluoroacetyl)isoindoline-5-amine

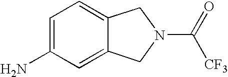

To a solution of 5-nitroisoindoline (1.06 g) and triethylamine (0.976 mL) in dichloromethane was added trifluoroacetic anhydride (0.989 mL) at 0° C. and the mixture was stirred at room temperature for 12 hrs. The reaction mixture was poured into saturated aqueous solution of sodium hydrogen carbonate and the mixture was extracted with ethyl acetate. The organic layer was dried (MgSO₄) and the solvent was evaporated. The obtained residue was purified by silica gel column chromatography (developing solvent: hexane/ethyl acetate=5/1-1/1) to give a pale yellow oil. To a suspension of the obtained residue and 10% palladium-carbon (0.25 g) in ethanol (20 mL) was slowly added hydrazine monohydrate (1.02 mL) at room temperature, and the mixture was stirred for 2 hrs. Palladium was removed by filtration, and the filtrate was concentrated. The residue was purified by aminopropyl silica gel column chromatography (developing solvent: hexane/ethyl acetate=2/1) to give the title compound as a white solid (0.98 g, yield 65%).

¹H NMR (300 MHz, CDCl₃) δ ppm: 3.13 (t, J=8.0 Hz, 2 H), 3.74 (s, 2 H), 4.18-4.31 (m, 2 H), 6.48 (dd, J=8.0, 2.2 Hz, 1 H), 7.02 (d, J=8.1 Hz, 1 H), 7.63 (d, J=2.1 Hz, 1 H).

The compounds described in the following Reference Examples 230-231 were produced in the similar manner as in Reference Example 229.

### Reference Example 230

### 2-(trifluoroacetyl)-1,2,3,4-tetrahydroisoquinoline-7-amine

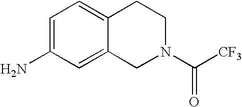

¹H NMR (300 MHz, DMSO-d₆) δ ppm: 2.91 (q, J=6.4 Hz, 2 H) 3.61 (s, 2 H), 3.77-3.88 (m, 2 H), 4.71-4.83 (m, 2 H), 7.07-7.21 (m, 2 H), 7.28 (d, J=7.9 Hz, 1 H).

### Reference Example 231

### 3-(trifluoroacetyl)-2,3,4,5-tetrahydro-1H-3-benzazepin-7-amine

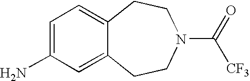

¹H NMR (300 MHz, CDCl₃) δ ppm: 2.81-2.92 (m, 4. H), 3.54-3.80 (m, 6 H), 6.45-6.54 (m, 2 H), 6.93 (t, J=7.8 Hz, 1 H).

### Reference Example 232

### methyl4-(hydroxymethyl)-2-nitrobenzoate

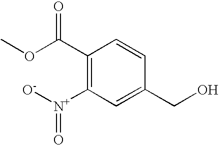

To a solution of 4-(methoxycarbonyl)-3-nitrobenzoic acid 20 (25.0 g, 111 mmol) in THF (250 mL) as added oxalyl chloride (11.5 mL, 133 mmol) under ice-cooling. DMF (3 mL) was added to the reaction solution and the mixture was stirred under ice-cooling for 1 hr. The organic solvent was removed by concentration under reduced pressure, and the residue was dissolved in DME (150 mL). This solution was added to a suspension of sodium borohydride (16.8 g, 444 mmol) in DME (100 mL) under ice-cooling, and the mixture was stirred for 4 hrs. 1N Hydrochloric acid was poured into the reaction mixture and the mixture was extracted with ethyl acetate. The organic layer was washed with saturated brine, dried (MgSO₄) and concentrated. The residue was purified by column chromatography to give the title compound as a colorless oil (23.4 g, yield 99%).

¹H NMR (300 MHz, CDCl₃) δ ppm: 3.92 (s, 3 H), 4.82 (d, J=6.0 Hz, 2 H), 7.60-7.71 (m, 2 H), 7.86 (s, 1 H).

### Reference Example 233

### methyl4-({[tert-butyl(dimethyl)silyl]oxy)methyl)-2-nitrobenzoate

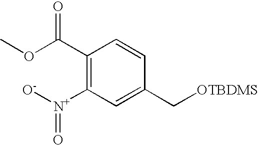

Methyl 4-(hydroxymethyl)-2-nitrobenzoate (23.4 g, 110 mmol) was dissolved in DMF (500 mL), and tert-butyl(dimethyl) silyl chloride (16.6 g, 110 mmol) and imidazole (7.5 g, 110 mmol) were added. The mixture was stirred at room temperature for 14 hrs. The reaction solution was poured into water and the mixture was extracted with ethyl acetate. The extract was washed with saturated brine, dried (MgSO₄) and the solvent was evaporated. The residue was purified by column chromatography to give the title compound (28.8 g, yield 81%).

¹H NMR (300 MHz, CDCl₃) δ ppm: 0.10 (s, 6 H), 0.94 (s, 9 H) 3.90 (s, 3 H), 4.81 (s, 2 H), 7.58 (dd, J=1.2, 8.1 Hz, 1 H), 7.70 (d, J=8.1 Hz, 1 H), 7.83 (d, J=1.2 Hz, 1 H).

### Reference Example 234

### 2-amino-4-[(dimethylamino)methyl)benzamide

### 1) 4-({[tert-butyl(dimethyl)silyl]oxy}methyl)-2-nitrobenzoic acid

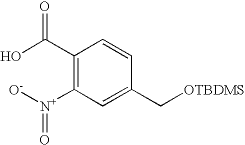

To a solution of methyl 4-({[tert-butyl(dimethyl) silyl]oxy}methyl)-2-nitrobenzoate (19.5 g, 60.0 mmol) in THF (50 mL)—methanol (50 mL) was added 1N aqueous sodium hydroxide solution (100 mL) and the mixture was stirred at room temperature for 14 hrs. 1N Hydrochloric acid was poured to acidify the reaction solution and the mixture was extracted with ethyl acetate. The extract was washed with saturated brine, dried (MgSO₄) and the solvent was evaporated under reduced pressure. The residue was crystallized from *diisopropyl ether to give 4-({[tert-butyl(dimethyl) silyl]oxy}methyl)-2-nitrobenzoic acid as white crystals (18.0 g, yield 96%).

¹H NMR (300 MHz, CDCl₃) δ ppm: 0.09 (s, 6 H), 0.92 (s, 9 H), 4.80 (s, 2 H), 7.58 (dd, J=1.2, 8.1 Hz, 1 H), 7.73 (d, J=1.2 Hz, 1 H), 7.83 (d, J=8.1 Hz, 1 H).

### 2) 4-({[tert-butyl(dimethyl) silyl]oxy}methyl)-2-nitrobenzamide

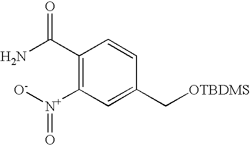

To a solution of 4-(([tert-butyl(dimethyl) silyl]oxy}methyl)-2-nitrobenzoic acid (7.0 g, 22.5 mmol) in DMF (200 mL) were added WSC (5.2 g, 27 mmol) and 1-hydroxy-1H-benzotriazoleammonium salt (4.1 g, 27 mmol), and the mixture was stirred at room temperature for 12 hrs. Water was poured into the reaction solution and the mixture was extracted with ethyl acetate. The extract was washed with 1N hydrochloric acid and saturated brine, dried (MgSO4) and concentrated under reduced pressure. The residue was purified by silica gel column chromatography to give 4-({[tert-butyl(dimethyl)silyl]oxy}methyl)-2-nitrobenzamide as white crystals (5.6 g, yield 80%).

¹H NMR (300 MHz, CDCl₃) δ ppm: 0. 11 (s, 6 H), 0.93 (s, 9 H), 4.79 (s, 2 H), 6.09(br, 1 H), 6.32(br, 1H), 7.51 (dd, J=1.2, 7.8 Hz, 1 H), 7.61 (d, J=7.8 Hz, 1H), 7.95 (d, J=1.2 Hz, 1H).

### 3) 4-[(dimethylamino)methyl]-2-nitrobenzamide

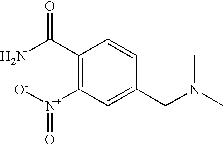

To a solution of 4-({[tert-butyl(dimethyl) silyl]oxy}methyl)-2-nitrobenzamide (3.0 g, 9.5 mmol) in THF (30 mL) was added 1.0 M tetrabutylammonium fluoride-THF solution (14 mL, 14 mmol) and the mixture was stirred at room temperature for 1 hr. Water was poured into the reaction solution and the mixture was extracted with ethyl acetate. The extract was washed with saturated brine, dried (MgSO₄) and concentrated under reduced pressure. The residue was dissolved in toluene (10 mL), and thionyl chloride (1.4 mL, 19 mmol) was added. The mixture was heated under reflux for 30 min. The reaction solution was cooled to room temperature, Water was poured into the reaction solution and the mixture was extracted with ethyl acetate. The extract was washed with saturated brine, dried (MgSO₄) and concentrated under reduced pressure. To the residue was added 2.0 M dimethylamine-THF solution (7 mL, 14 mmol) and the mixture was stirred at 50° C. for 6 hrs. Water was poured into the reaction solution and the mixture was extracted with ethyl acetate. The extract was washed with saturated brine, dried (MgSO₄) and concentrated under reduced pressure. The residue was purified by silica gel column chromatography to give 4-[(dimethylamino)methyl]-2-nitrobenzamide as white crystals (2.1 g, yield 89%).

¹H NMR (300 MHz, CDCl₃) δ ppm: 2.25 (s, 6 H), 3.51 (s, 2 H), 5.88 (br, 2 H), 7.52 (d, J=7.8 Hz, 1 H), 7.63 (dd, J=1.2, 7.8 Hz, 1 H), 8.02 (d, J=1.2 Hz, 1 H).

### 4) 2-amino-4-[(dimethylamino)methyl]benzamide

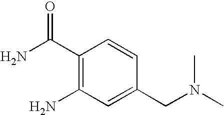

To a solution of 4-[(dimethylamino)methyl]-2-nitrobenzamide (0.7 g, 3.1 mmol) in ethanol (5 mL) was added 5% palladium-carbon (70 mg) and hydrazine monohydrate (0.5 mL, 9.5 mmol) was slowly added dropwise under ice-cooling. The temperature of the mixture was gradually raised to room temperature and the mixture was stirred for 3 hrs. Palladium carbon was removed by filtration. The filtrate was concentrated and purified by silica gel column chromatography to give 2-amino-4-[(dimethylamino)methyl]benzamide as white crystals (0.39 g, yield 65%).

¹H NMR (300 MHz, CDCl₃) δ ppm: 2.23 (s, 6 H), 3.32 (s, 2 H) 5.69 (br, 2 H), 6.61 (dd, J=1.2, 8.1 Hz, 1 H), 6.65 (d, J=1.2 Hz, 1 H), 7.30 (d, J=8.1 Hz, 1 H).

The compounds described in the following Reference Examples 235-238 were produced in the similar manner as in Reference Example 234.

### Reference Example 235

### 2-amino-4-[(dimethylamino)methyl]-N-methylbenzamide

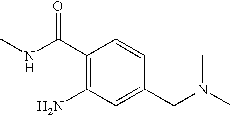

¹H NMR (300 MHz, CDCl₃) δ ppm: 2.22 (s, 6 H), 2.95 (dd, J=2.1, 4.8 Hz, 3 H), 3.30 (s, 2 H), 5.50(br, 2 H), 6.05 (br, 1 H), 6.58 (dd, J=1.8, 8.1 Hz, 1 H), 6.64 (d, J=1.8 Hz, 1 H), 7.23 (d, J=8.1 Hz, 1 H).

### Reference Example 236

### 2-amino-4-[(dimethylamino)methyl]-N-ethylbenzamide

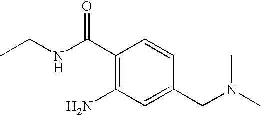

¹H NMR (300 MHz, CDCl₃) δ ppm: 1.13 (t, J=7.2 Hz, 3 H), 2.22 (s, 6 H), 3.31 (s, 2 H), 3.45(q, J=7.2 Hz, 2 H), 5.49(br, 2 H), 6.00(br, 1 H), 6.58 (dd, J=1.5, 8.1 Hz, 1 H), 6.64 (d, J=1.5 Hz, 1 H), 7.24 (d, J=8.1 Hz, 1 H).

### Reference Example 237

### 2-amino-4-[(dimethylamino)methyl]-N,N-dimethylbenzamide

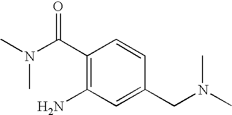

¹H NMR (300 MHz, CDCl₃) δ ppm: 2.19 (s, 6 H), 3.01 (s, 6 H), 3.28 (s, 2 H), 4.31 (br, 2 H), 6.60 (dd, J=1.2, 7.8 Hz, 1 H), 6.65 (d, J=1.2 Hz, 1 H), 7.00 (d, J=7.8 Hz, 1 H).

### Reference Example 238

### [3-amino-4-(pyrrolidin-1-ylcarbonyl)benzyl]dimethylamine

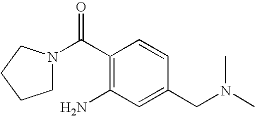

¹H NMR (300 MHz, CDCl₃) δ ppm: 1.81-1.93 (m, 4 H), 2.23 (s, 6 H), 3.31 (s, 2 H), 3.42-3.68(m, 4 H), 4.62(br, 2 H), 6.62 (dd, J=1.2, 7.8 Hz, 1 H), 6.67 (d, J=1.2 Hz, 1 H), 7.15 (d, J=7.8 Hz, 1 H).

### Reference Example 239

### methyl 4-(chloromethyl)-2-nitrobenzoate

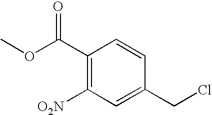

A mixture of methyl 4-(hydroxymethyl)-2-nitrobenzoate (7.11 g), pyridine (0.456 g) and thionyl chloride (4.92 g) in diethyl ether (200 mL)-THF (50 mL) was stirred at room temperature for 16 hrs. The reaction mixture was washed with 1N hydrochloric acid and a saturated aqueous solution of sodium hydrogen carbonate, dried (MgSO₄) and the solvent was evaporated. The obtained residue was washed with isopropyl ether-hexane to give the title compound as a white powder (6.94 g, yield 90%).

¹H NMR (300 MHz, CDCl₃) δ ppm: 3.93 (s, 3 H), 4.65 (s, 2 H), 7.70 (dd, J=7.8, 1.5 Hz, 1 H), 7.76 (d, J=7.8 Hz, 1 H), 7.94 (d, J=1.32 Hz, 1 H)

The compound described in the following Reference Example 240 was produced in the similar manner as in Reference Example 9.

### Reference Example 240

### methyl 4-[(dimethylamino)methyl]-2-nitrobenzoate

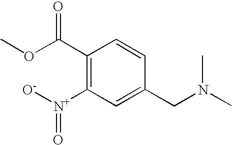

¹H NMR (300 MHz, CDCl₃) δ ppm: 2.21 (s, 6 H), 3.47 (s, 2 H) 3.87 (s, 3 H), 7.55 (dd, J=1.0, 8.2 Hz, 1 H), 7.66 (d, J=8.2 Hz, 1 H), 7.81 (d, J=1.0 Hz, 1 H).

The compound described in the following Reference Example 241 was produced in the similar manner as in Reference Example 10.

### Reference Example 241

### methyl 4-[(dimethylamino)methyl]-2-nitrobenzoate

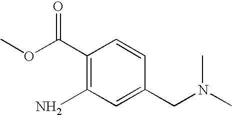

¹H NMR (300 MHz, CDCl₃) δ ppm: 2.65 (3H, s) 2.67 (3H, s) 3.47 (2H, s) 3.78 (3H, s) 4.12(2H, d, J=6.0 Hz) 6.73 (1H, dd, J=1.2, 7.5Hz) 6.84 (1H, d, J=1.2 Hz) 7.73 (1H, d, J=7.5 Hz).

### Reference Example 242

### 4-(1,3-dioxolan-2-yl)-2-nitrophenyl trifluoromethanesulfonate

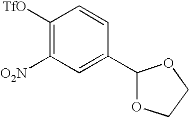

To a solution of 4-hydroxy-3-nitrobenzaldehyde (15 g, 90 mmol) and triethylamine (15 mL, 110 mmol) in dichloromethane (150 mL) was added dropwise trifluoromethanesulfonic anhydride (31 g, 110 mmol) under ice-cooling over 10 min. The mixture was stirred at 0° C. for 30 min. and water was added to the reaction solution. The mixture was extracted with ethyl acetate. The extract was washed with saturated brine, dried (MgSO₄) under reduced pressure and the solvent was evaporated. The residue was dissolved in toluene, and ethylene glycol (56 g, 890 mmol) and methanesulfonic acid (0.2 mL, 2 mmol) were added. The mixture was heated under reflux for 14 hrs. using a Dean-Stark trap. The reaction solution was separated into ethyl acetate and saturated aqueous solution of sodium hydrogen carbonate. The organic layer was washed successively with saturated aqueous solution of sodium hydrogen carbonate (50 mL) and then saturated brine (50 mL), and dried (MgSO₄). The solvent was evaporated and the residue was purified by silica gel column chromatography to give the title compound as pale yellow crystals (30.0 g, yield 99%).

¹H NMR (300 MHz, CDCl₃) δ ppm: 4.05-4.15 (m, 4 H), 5.89 (s, 1 H), 7.46 (d, J=8.7 Hz, 1 H), 7.84 (dd, J=2.4, 8.7 Hz, 1 H), 8.28 (d, J=2.5 Hz, 1 H).

### Reference Example 243

### 2-[3-nitro-4-(2-thienyl)phenyl]-1,3-dioxolane

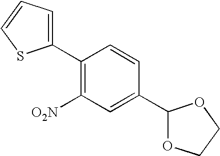

4-(1,3-Dioxolan-2-yl)-2-nitrophenyl trifluoromethanesulfonate (3.4 g, 10 mmol), sodium hydrogen carbonate (2.5 g, 30 mmol) and 2-thienylboronic acid (3.8 g, 30 mmol) were dissolved in DME (100 mL)—water (10 mL). To the reaction solution was added tetrakistriphenylphosphine palladium (550 mg, 0.5 mmol) and the mixture was stirred under an argon atmosphere at 40° C. for 13 hrs. Water was added to the reaction solution and the mixture was extracted with ethyl acetate. The extract was washed with saturated brine, dried (MgSO₄) under reduced pressure and the solvent was evaporated. The residue was purified by silica gel column chromatography to give the title compound as a pale yellow oil (2.0 g, yield 72%).

¹H NMR (300 MHz, CDCl₃) δ ppm: 4.05-4.18 (m, 4 H), 5.89 (s, 1 H), 7.06-7.12 (m, 2 H), 7.42 (dd, J=6.0, 1.8 Hz, 1 H), 7.57 (d, J=7.2 Hz, 1 H), 7.66 (dd, J=1.2, 7.2 Hz, 1 H), 7.88 (d, J=1.2 Hz, 1 H).

### Reference Example 244

### N,N-dimethyl-1-[3-nitro-4-(2-thienyl)phenyl]methanamine

### 1) 3-nitro-4-(2-thienyl)benzaldehyde

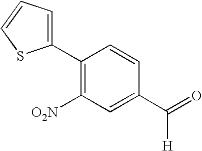

2-[3-Nitro-4-(2-thienyl)phenyl]-1,3-dioxolane (2.0 g, 7.2 mmol) was dissolved in THF (10 mL)—1N hydrochloric acid (10 mL), and the mixture was stirred at room temperature for 12 hrs. Water was added to the reaction solution and the mixture was extracted with ethyl acetate. The extract was washed with saturated brine, dried (MgSO₄) and the solvent was evaporated under reduced pressure to give 3-nitro-4-(2-thienyl)benzaldehyde as pale yellow crystals (1.65 g, yield 98%).

¹H NMR (300 MHz, CDCl₃) δ ppm: 7.10 (dd, J=3.6, 4.8 Hz, 1 H), 7.18 (dd, J=1.2, 3.6 Hz, 1 H), 7.50 (dd, J=7.2, 1.2 Hz, 1 H), 7.74 (d, J=7.8 Hz, 1 H), 8.05 (dd, J=7.8, 1.5 Hz, 1 H), 8.18 (d, J=1.5 Hz, 1 H), 10.05 (s, 1 H).

### 2) N,N-dimethyl-1-[3-nitro-4-(2-thienyl)phenyl]methanamine

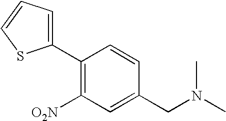

To a solution of 3-nitro-4-(2-thienyl)benzaldehyde (1.6 g, 7.0 mmol) in THF (5 mL) was added 2.0 M dimethylamine-THF solution (5 mL, 10 mmol) and the mixture was stirred at room temperature. After 30 min., sodium triacetoxyborohydride (1.8 g, 8.4 mmol) was added to the reaction solution and the mixture was further stirred for 30 min. To the reaction solution was added a saturated aqueous solution of sodium hydrogen carbonate solution and the mixture was extracted with ethyl acetate. The extract was washed with saturated brine, dried (MgSO₄) and the solvent was evaporated under reduced pressure to give N,N-dimethyl-1-[3-nitro-4-(2-thienyl)phenyl]methanamine as a pale yellow oil (1.69 g, yield 92%).

¹H NMR (300 MHz, CDCl₃) δ ppm: 2.27 (s, 6 H), 3.48 (s, 2 H) 7.04-7.08 (m, 2 H), 7.37-7.41 (m, 1 H), 7.46-7.55 (m, 2 H), 7.71-7.72 (m, 1 H).

### Reference Example 245

### [3-amino-4-(2-thienyl)benzyl]dimethylamine dihydrochloride

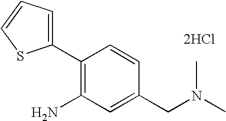

To a solution of N,N-dimethyl-1-[3-nitro-4-(2-thienyl)phenyl]methanamine (1.6 g, 6.0 mmol) in ethanol (50 mL) was added 5% palladium-carbon (160 mg). To the reaction solution was slowly added dropwise hydrazine monohydrate (0.85 mL, 18 mmol). After the completion of the dropwise addition, the mixture was stirred at 70° C. for 2 hrs. After cooling the reaction solution to room temperature, palladium carbon was removed, and the filtrate was concentrated. The residue was purified by silica gel column chromatography and crystallized from 4N hydrochloric acid-ethyl acetate solution to give the title compound as pale yellow crystals (0.86 g, yield 46%).

¹H NMR (300 MHz, DMSO-d₆) δ ppm: 2.46 (s, 6 H), 3.98 (s, 2 H), 6.93-6.98 (m, 3 H), 7.15-7.21 (m, 2 H), 7.41-7.43 (m, 1 H).

The compound described in the following Reference Example 246 was produced in the similar manner as in Reference Example 243.

### Reference Example 246

### 4-[4-(1,3-dioxolan-2-yl)-2-nitrophenyl]-1H-pyrazole

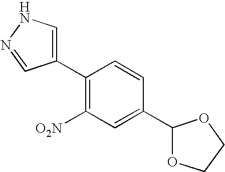

¹H NMR (300 MHz, CDCl₃) δ ppm: 4.05-4.18 (m, 4 H), 5.89 (s, 1 H), 7.52 (d, J=7.8 Hz, 1 H), 7.67 (dd, J=1.8, 7.8 Hz, 1 H) 7.76 (s, 2 H), 7.88 (d, J=1.8 Hz, 1 H).

### Reference Example 247

### 4-[4-(1,3-dioxolan-2-yl)-2-nitrophenyl]-1-methyl-1H-pyrazole

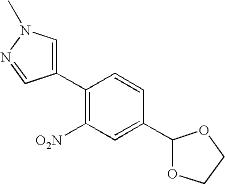

To a solution of 18-crown-6 (160 mg, 0.6 mmol) in diethyl ether (5 mL) was added potassium tert-butoxide (810 mg, 0.72 mmol) and the mixture was stirred at room temperature for 90 min. 4-[4-(1,3-Dioxolan-2-yl)-2-nitrophenyl]-1H-pyrazole (1.6 g, 6.1 mmol) and iodomethane (0.45 mL, 7.2 mmol) were added and the mixture was stirred at room temperature for 3 hrs. Water was added to the reaction solution and the mixture was extracted with ethyl acetate. The extract was washed with saturated brine, dried (MgSO₄), and purified. The residue was applied to silica gel column chromatography to give the title compound as pale yellow crystals (1.1 g, yield 66%).

¹H NMR (300 MHz, CDCl₃) δ ppm: 3.94 (s, 3 H), 4.04-4.16 (m, 4 H), 5.87 (s, 1 H), 7.49 (d, J=8.1 Hz, 1 H), 7.54 (s, 1 H), 7.60 (d, J=1.0 Hz, 1 H), 7.63 (dd, J=1.0, 7.8 Hz, 1 H), 7.83 (dd, J=1.0 Hz, 1 H).

The compound described in the following Reference Example 248 was produced in the similar manner as in Reference Example 244.

### Reference Example 248

### N,N-dimethyl-1-[4-(1-methyl-1H-pyrazole-4-yl)-3-nitrophenyl]methanamine

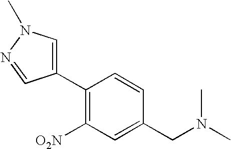

¹H NMR (300 MHz, CDCl₃) δ ppm: 2.26 (s, 6 H), 3.48 (s, 2 H), 3.93 (s, 3 H), 7.04-7.08 (m, 2 H), 7.41 (d, J=7.8 Hz, 1 H), 7.50 (dd, J=1.8, 7.8 Hz, 1 H), 7.53 (s, 1 H), 7.60 (s, 1 H), 7.67 (d, J=1.8 Hz, 1 H)

The compound-described in the following Reference Example 249 was produced in the similar manner as in Reference Example 245.

### Reference Example 249

### [3-amino-4-(2-thienyl)benzyl]dimethylamine dihydrochloride

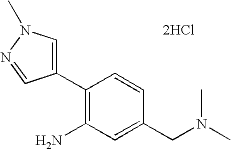

¹H NMR (300 MHz, DMSO-d₆) δ ppm: 2.48 (s, 6 H), 2.69 (s, 3 H), 3.89 (s, 2 H), 6.93-6.98 (m, 3 H), 7.13-7.47 (m, 3 H), 7.81-7.83 (m, 1 H), 8.14-8.16 (m, 1 H)

The compound described in the following Reference Example 250 was produced in the similar manner as in Reference Example 243 and by processing the obtained compound in the similar manner as in Reference Example 244 (1).

### Reference Example 250

### 3-nitro-4-(2-furyl)benzaldehyde

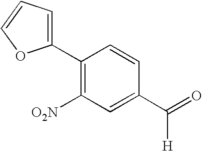

¹H NMR (300 MHz, CDCl₃) δ ppm: 6.56 (dd, J=1.8, 3.6 Hz,1 H), 6.85 (dd, J=0.9, 3.6 Hz, 1 H), 7.59 (dd, J=0.9, 1.8 Hz, 1 H), 7.94 (d, J=8.4 Hz, 1 H), 8.06 (dd, J=8.4, 1.5 Hz, 1 H), 8.11 (d, J=1.5 Hz, 1 H), 10.03 (s, 1 H).

The compound described in the following Reference Example 251 was produced in the similar manner as in Reference Example 244 (2).

### Reference Example 251

### N,N-dimethyl-1-[3-nitro-4-(2-furyl)phenyl]methanamine

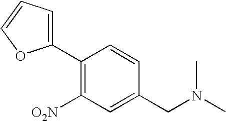

¹H NMR (300 MHz, CDCl₃) δ ppm: 2.22 (s, 6 H), 3.44 (s, 2 H), 6.45 (dd, J=2.7, 4.8 Hz, 1 H), 6.60 (d, J=4.8 Hz, 1 H), 7.46-7.52 (m, 2 H), 7.59-7.64 (m, 2 H).

The compound described in the following Reference Example 252 was produced in the similar manner as in Reference Example 245.

### Reference Example 252

### [3-amino-4-(2-furyl)benzyl]dimethylamine dihydrochloride

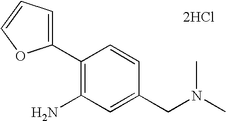

¹H NMR (360 MHz, DMSO-d₆) δ ppm: 2.46 (s, 6 H), 4.13 (s, 2 H), 6.61-6.63 (m, 1 H), 6.84-6.99 (m, 3 H), 7.51-7.57 (m, 1 H), 7.76-7.77 (m, 1 H).

The compound described in the following Reference Example 253 was produced in the similar manner as in Reference Example 243.

### Reference Example 253

### 2-[3-nitro-4-(3-thienyl)phenyl]-1,3-dioxolane

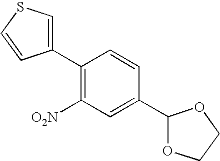

¹H NMR (300 MHz, CDCl₃) δ ppm: 4.05-4.18 (m, 4 H), 5.89 (s, 1 H), 7.06 (dd, J=1.5, 8.1 Hz, 2 H), 7.33 (dd, J=1.5, 9.0 Hz, 1 H), 7.38 (dd, J=4.8, 3.0 Hz, 1 H), 7.50 (d, J=7.8 Hz, 1 H), 7.67 (ddd, J=0.6, 8.1, 1.5 Hz, 1 H), 7.91 (d, J=1.8 Hz, 1 H).

The compound described in the following Reference Example 254 was produced in the similar manner as in Reference Example 244.

### Reference Example 254

### N,N-dimethyl-1-[3-nitro-4-(3-thienyl)phenyl)methanamine

### 1) 3-nitro-4-(3-thienyl)benzaldehyde

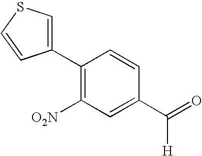

¹H NMR (300 MHz, CDCl₃) δ ppm: 7.11 (dd, J=1.8, 4.8 Hz, 1 H), 7.42-7.46 (m, 2 H), 7.70 (d, J=8.1 Hz, 1 H), 8.09 (dd, J=8.1, 1.8 Hz, 1 H), 8.26 (d, J=1.8 Hz, 1 H), 10.08 (s, 1 H).

### 2) N,N-dimethyl-1-[3-nitro-4-(3-thienyl)phenyl]methanamine

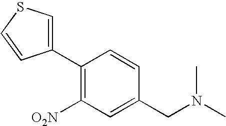

¹H NMR (300 MHz, CDCl₃) δ ppm: 2.28 (s, 6 H), 3.49 (s, 2 H), 7.07 (dd, J=1.5, 4.8 0Hz, 1 H), 7.31 (dd, J=1.5, 3.0 Hz, 1 H), 7.37 (dd, J=3.0, 4.8 Hz, 1 H), 7.43 (d, J=8.1 Hz, 1 H), 7.53 (dd, J=1.8, 8.1 Hz, 1 H), 7.75 (d, J=1.8 Hz, 1 H).

The compound described in the following Reference Example 255 was produced in the similar manner as in Reference Example 10.

### Reference Example 255

### [3-amino-4-(3-thienyl)benzyl]dimethylamine

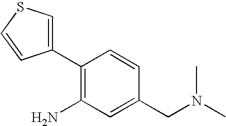

¹H NMR (300 MHz, CDCl₃) δ ppm: 2.27 (s, 6 H), 3.36 (s, 2 H), 6.70-6.75 (m, 2 H), 7.14 (d, J=7.5 Hz, 1 H), 7.24-7.26 (m, 1 H), 7.35-7.37 (m, 1 H), 7.39-7.42 (m, 1 H).

### Reference Example 256

### 2-[4-(methylthio)-3-nitrophenyl]-1,3-dioxolane

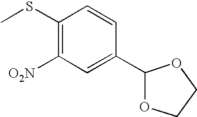

To a solution of 4-(1,3-dioxolan-2-yl)-2-nitrophenyl trifluoromethanesulfonate (3.4 g, 10 mmol) in ethanol (50 mL) was added sodium methanethiolate (1.1 g, 15 mmol) and the mixture was heated under reflux for 20 min. The reaction solution was cooled to room temperature, water was added and the mixture was extracted with ethyl acetate. The extract was washed with saturated brine, dried (MgSO₄) under reduced pressure and the solvent was evaporated. The residue was purified by silica gel column chromatography to give the title compound as yellow crystals (2.3 g, yield 95%).

¹H NMR (300 MHz, CDCl₃) δ ppm: 2.52 (s, 3 H), 4.01-4.17 (m, 4 H), 5.85 (s, 1 H), 7.38 (d, J=8.1 Hz, 1 H), 7.68 (dd, J=2.1, 8.1 Hz, 1 H), 8.38 (d, J=2.1 Hz, 1 H).

The compound described in the following Reference Example 257 was produced in the similar manner as in Reference Example 244 (1).

### Reference Example 257

### 4-(methylthio)-3-nitrobenzaldehyde

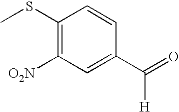

¹H NMR (300 MHz, CDCl₃) δ ppm: 2.59 (s, 3 H), 7.53 (d, J=7.8 Hz, 1 H), 8.07 (dd, J=1.5, 7.8 Hz, 1 H), 8.72 (d, J=1.5 Hz, 1 H), 10.01 (s, 1 H).

The compound described in the following Reference Example 25 258 was produced in the similar manner as in Reference Example 244 (2).

### Reference Example 258

### N,N-dimethyl-1-[4-(methylthio)-3-nitrophenyl]methanamine

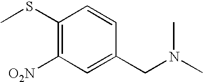

¹H NMR (300 MHz, CDCl₃) δ ppm: 2.21 (s, 6 H), 3.41 (s, 2 H), 7.28 (d, J=8.4 Hz, 1 H), 7.37-7.41 (m, 1 H), 7.52 (dd, J=2.7, 8.4 Hz, 1 H), 8.14 (d, J=2.7 Hz, 1 H).

The compound described in the following Reference Example 259 was produced in the similar manner as in Reference Example 10.

### Reference Example 259

### [3-amino-4-(methylthio)benzyl]dimethylamine

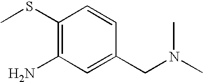

¹H NMR (300 MHz, CDCl₃) δ ppm: 2.18 (s, 6 H), 2.29 (s, 3 H), 3.26 (s, 2 H), 4.20 (br, 2 H), 6.59 (dd, J=1.8, 8.4 Hz, 1 H), 6.65 (d, J=1.8 Hz, 1 H), 7.24 (d, J=8.4 Hz, 1H).

### Reference Example 260

### [3-amino-4-(methylsulfinyl)benzyl]dimethylamine and [3-amino-4-(methylsulfonyl)benzyl]dimethylamine

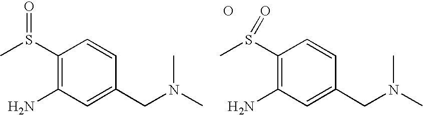

To a solution of [3-amino-4-(methylthio)benzyl]dimethylamine (1.5 g, 7.6 mmol) in dichloromethane (100 mL) was added m-chloroperbenzoic acid (5.6 g, 22.9 mmol) under ice-cooling and the mixture was stirred for 2 hrs. The temperature of the reaction solution was raised to room temperature and aqueous sodium sulfite solution was added. The mixture was stirred for 30 min and the reaction solution was extracted with ethyl acetate. The extract was washed with saturated brine, dried (MgSO₄), and the solvent was evaporated under reduced pressure. The residue was purified by silica gel column chromatography to give both [3-amino-4-(methylsulfinyl)benzyl]dimethylamine (0.2 g, yield 12%) and [3-amino-4-(methylsulfonyl)benzyl]dimethylamine (0.8 g, yield 46%).

### [3-amino-4-(methylsulfinyl)benzyl]dimethylamine

¹H NMR (300 MHz, CDCl₃) δ ppm: 2.19 (s, 6 H), 2.87 (s, 3 H), 3.29 (s, 2 H), 4.97 (br, 2 H), 6.62-6.66 (m, 2 H), 7.12 (d, J=8.4 Hz, 1 H).

### [3-amino-4-(methylsulfonyl)benzyl]dimethylamine

¹H NMR (300 MHz, CDCl₃) δ ppm: 2.20 (s, 6 H), 3.01 (s, 3 H) 3.17 (5, 2 H), 4.97 (br, 2 H), 6.67-6.74 (m, 2 H), 7.62 (d, J=8.4 Hz, 1 H).

The compound described in the following Reference Example 261 was produced in the similar manner as in Reference Example 256.

### Reference Example 261

### 2-[4-(isopropylthio)-3-nitrophenyl]-1,3-dioxolane

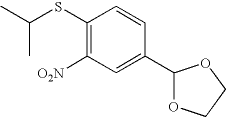

¹H NMR (300 MHz, CDCl₃) δ ppm: 1.39 (d, J=6.6 Hz, 6 H), 3.59 (sep, J=6.6 Hz, 1 H), 4.04-4.15 (m, 4 H), 5.83 (s, 1 H), 7.46 (d, J=8.1 Hz, 1 H), 7.62 (dd, J=1.8, 8.1 Hz, 1 H), 8.23 (d, J=1.8 Hz, 1 H).

The compound described in the following Reference Example 262 was produced in the similar manner as in Reference Example 244.

### Reference Example 262

N,N-dimethyl-1-[4-(isopropylthio)-3-nitrophenyl]methanamine

### 1) 4-(isopropylthio)-3-nitrobenzaldehyde

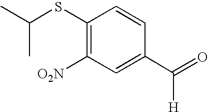

¹H NMR (300 MHz, -CDCl₃) δ ppm: 1.26 (d, J=6.6 Hz, 6 H), 3.66 (sep, J=6.6 Hz, 1 H), 7.59 (d, J=7.8 Hz, 1 H), 8.02 (dd, J=1.5, 7.8 Hz, 1 H), 8.63 (d, J=1.5 Hz, 1 H), 9.99 (s, 1 H).

### 2) N,N-dimethyl-1-[4-(isopropylthio)-3-nitrophenyl]methanamine

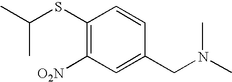

¹H NMR (300 MHz, CDCl₃) δ ppm: 1.38 (d, J=6.6 Hz, 6 H), 2.25 (s, 6 H), 3.44 (s, 2 H), 3.58 (sep, J=6.6 Hz, 1 H), 7.41 (d, J=8.4 Hz, 1 H), 7.50 (dd, J=2.1, 8.4 Hz, 1 H), 8.04 (d, J=2.1 Hz, 1 H).

The compound described in the following Reference Example 263 was produced in the similar manner as in Reference Example 10.

### Reference Example 263

### [3-amino-4-(isopropylthio)benzyl]dimethylamine

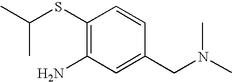

¹H NMR (300 MHz, CDCl₃) δ ppm: 1.22 (d, J=6.6 Hz, 6 H), 2.24 (s, 6 H), 3.26 (s, 2 H), 3.17 (sep, J=6.6 Hz, 1 H), 3.30 (s, 2 H), 4.35 (br, 2 H), 6.61 (dd, J=1.8, 8.4 Hz, 1 H), 6.71 (d, J=1.8 Hz, 1 H), 7.29 (d, J=8.4 Hz, 1 H).

### Reference Example 264

### 3-oxo-3,4-dihydro-2H-1,4-benzoxazin-6-carboxylic acid

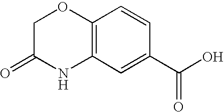

To a solution of 3-amino-4-hydroxybenzoic acid (10 g, 66 mmol) in THF (25 mL) were added potassium carbonate (14.0 g, 100 mmol) and water (50 mL) and chloroacetyl chloride (8.0 mL, 100 mL) was added. The mixture was stirred at room temperature for 3 hrs. Concentrated hydrochloric acid was added to acidify the reaction solution. The precipitated crystals were collected by filtration. The obtained crystals were washed with water and diisopropyl ether and vacuum dried to give the title compound as brown crystals (9.4 g, yield 74%).

¹H NMR (300 MHz, DMSO-d₆) δ ppm: 4.65 (s, 2 H), 7.00 (d, J=8.4 Hz, 1 H), 7.48-7.52 (m, 2 H), 10.87 (s, 1 H), 12.77 (br, 5 1 H).

### Reference Example 265

### N,N-dimethyl-3-oxo-3,4-dihydro-2H-1,4-benzoxazin-6-carboxamide

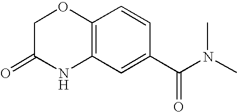

To a solution of 3-oxo-3,4-dihydro-2H-1,4-benzoxazin-6-carboxylic acid (3.8 g, 20 mmol) in DMF (100 mL) were added WSC (5.7 g, 30 mmol), HOBt (5.1 g, 30 mmol) and 2.0 M dimethylamine-THF solution (15 mL, 30 mmol) and the mixture was stirred at room temperature for 12 hrs. A saturated aqueous solution of sodium hydrogen carbonate was poured into the reaction solution and the mixture was extracted with ethyl acetate. The extract was washed with 1N hydrochloric acid and saturated brine, dried (MgSO₄) and concentrated under reduced pressure. The residue was crystallized from diisopropyl ether to give the title compound (3.8 g, yield 87%) as pale yellow crystals.

¹H NMR (300 MHz, CDCl₃) δ ppm: 3.05 (br, 6 H), 4.63 (s, 2 H), 6.95 (d, J=8.1 Hz, 1 H), 7.02 (dd, J=1.8, 8.1 Hz, 1 H), 7.08 (d, J=1.8 Hz, 1 H), 8.85 (s, 1 H).

### Reference Example 266

### (3,4-dihydro-2H-1,4-benzoxazin-6-ylmethyl)dimethylamine dihydrochloride

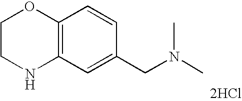

N,N-Dimethyl-3-oxo-3,4-dihydro-2H-1,4-benzoxazin-6-carboxamide (2.0 g, 9.1 mmol) was dissolved in 1.1 M borane-THF solution (60 mL, 66 mmol), and the mixture was heated under reflux for 12 hrs. Methanol (30 mL) was slowly added to the reaction solution and the mixture was further heated under reflux for 16 hrs. The solvent was evaporated under reduced pressure, and the residue was crystallized from 4N hydrochloric acid-ethyl acetate solution to give the title compound as white crystals (1.88 g, yield 78%).

¹H NMR (300 MHz, DMSO-d₆) δ ppm: 2.45 (s, 3 H), 2.47 (s, 3 H), 3.12 (s, 2 H), 3.85-.3.87 (m, 2 H), 3.95-3.98 (m, 2 H), 6.55-6.59 (m, 3 H), 10.07 (br, 1 H).

### Reference Example 267

### 4-[(2-ethoxy-2-oxoethyl)thio]-3-nitrobenzoic acid

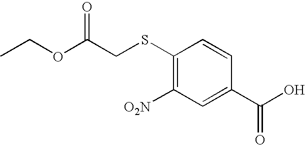

To a solution of 4-chloro-3-nitrobenzoic acid (15 g, 74 mmol) in pyridine (50 mL) was added mercaptoethyl acetate (10.8 g, 90 mmol) and the mixture was heated under reflux for 15 hrs. After cooling the reaction solution to room temperature, concentrated hydrochloric acid was added to acidify the solution. The precipitated crystals were collected by filtration. The obtained crystals were washed with water and vacuum dried to give the title compound as orange crystals (1.88 g, yield 78%).

¹H NMR (300 MHz, DMSO-d₆) δ ppm: 1.18 (t, J=7.2 Hz, 3 H) 4.15 (q, J=7.2 Hz, 2 H), 4.21 (s, 2 H), 7.69 (d, J=8.7 Hz, 1 H), 8.15 (dd, J=8.7, 1.8 Hz, 1 H), 8.63 (d, J=1.8 Hz, 1 H).

### Reference Example 268

### ethyl 4-[(2-ethoxy-2-oxoethyl)thiol-3-nitrobenzoate

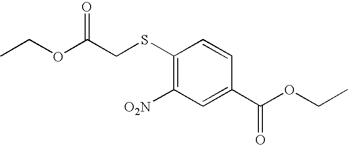

To a solution of 4-[(2-ethoxy-2-oxoethyl)thio]-3-nitrobenzoic acid (16.5 g, 58 mmol) in ethanol (200 mL) was added sulfuric acid (30 mL) and the mixture was stirred at 80° C. for 1.5 hrs. The reaction solution was cooled to room temperature, and water was added. The mixture was extracted with ethyl acetate. The extract was washed with saturated brine, dried (MgSO₄), and the solvent was evaporated under reduced pressure. The residue was purified by silica gel column chromatography to give the title compound as yellow crystals (12.1 g, yield 67%).

¹H NMR (300 MHz, CDCl₃) δ ppm: 1.27 (t, J=7.2 Hz, 3 H), 1.42 (t, J=7.2 Hz, 3 H), 3.79 *(s, 2 H), 4.22 (q, J=7.2 Hz, 2 H), 4.43 (q, J=7.2 Hz, 2 H), 7.57 (d, J=8.4 Hz, 1 H), 8.20 (dd, J=8.4, 2.7 Hz, 1 H), 8.87 (d, J=2.7 Hz, 1 H).

### Reference Example 269

### ethyl 3-oxo-3,4-dihydro-2H-1,4-benzothiazin-6-carboxylate

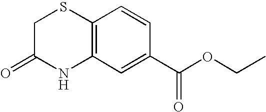

To a solution of ethyl 4-[(2-ethoxy-2-oxoethyl)thio]-3-nitrobenzoate (12.6 g, 40 mmol) in ethanol (120 mL) was added 5% palladium-carbon (1.3 g) and the mixture was stirred under a hydrogen atmosphere (0.5 MPa) at 50° C. for 7 hrs. Palladium carbon was collected by filtration, and the filtrate was concentrated. The residue was dissolved in ethanol (100 mL), and triethylamine (7 mL, 50 mmol) was added. The mixture was stirred at 60° C. for 2 hrs. The reaction solution was concentrated under reduced pressure and the residue was purified by silica gel column chromatography to give the title compound as pale yellow crystals (5.12 g, yield 54%).

¹H NMR (300 MHz, CDCl₃) δ ppm: 1.37 (t, J=7.2 Hz, 3 H), 3.43 (s, 2 H), 4.35 (q, J=7.2 Hz, 2 H), 7.34-(d, J=8.0 Hz, 1 H), 7.50 (d, J=1.8 Hz, 1 H), 7.64 (J=8.0, 1.8 Hz, 1 H), 8.22 (br, 1 H).

### Reference Example 270

### 3-oxo-3,4-dihydro-2H-1,4-benzothiazin-6-carboxylic acid

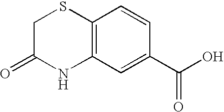

To a solution of ethyl 3-oxo-3,4-dihydro-2H-1,4-benzothiazin-6-carboxylate (5.1 g, 21 mmol) in THF (30 mL) was added 1N aqueous sodium hydroxide solution (100 mL) and the mixture was stirred at room temperature for 3 hrs. The reaction solution was acidified by pouring 1N hydrochloric acid thereinto and the mixture was extracted with ethyl acetate. The extract was washed with saturated brine, dried (MgSO₄) and the solvent, was evaporated under reduced pressure. The residue was crystallized from diisopropyl ether to give the title compound as white crystals (3.65 g, yield 83%).

¹H NMR (300 MHz, DMSO-d₆) δ ppm: 3.26 (br, 1 H), 3.48 (s, 2 H), 7.34-7.51 (m, 3 H), 10.69 (br, 1 H).

### Reference Example 271

### N-dimethyl-3-oxo-3,4-dihydro-2H-1,4-benzothiazin-6-carboxamide

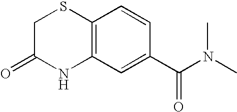

To a solution of 3-oxo-3,4-dihydro-2H-1,4-benzothiazin-6-carboxylic acid (3.6 g, 17 mmol) in DMF (50 mL) were added WSC 3.8 g, 20 mmol), HOBt (3.1 g, 20 mmol) and 2.0 M dimethylamine-THF solution (12.5 mL, 25 mmol), and the mixture was stirred at room temperature for 12 hrs. Saturated aqueous sodium hydrogen carbonate was poured into the reaction solution and the mixture was extracted with ethyl acetate. The extract was washed with 1N hydrochloric acid and saturated brine, dried (MgSO₄) and concentrated under reduced pressure. The residue was crystallized from diisopropyl ether to give the title compound as pale yellow crystals (3.6 g, yield 90%).

¹H NMR (300 MHz, DMSO-d₆) δ ppm: 2.91 (br, 6 H), 3.49 (s, 2 H), 6.96-7.00 (m, 2 H), 7.35 (d, J=8.4 Hz, 1 H).

### Reference Example 272

### (3,4-dihydro-2H-1,4-benzothiazin-6-ylmethyl)dimethylamine

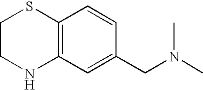

N,N-Dimethyl-3-oxo-3,4-dihydro-2H-1,4-benzothiazin-6-carboxamide (3.6 g, 15 mmol) was added to 1.0 M borane-THF solution (150 mL, 150 mmol) and the mixture was heated under reflux for 12 hrs. Methanol (50 mL) was slowly added to the reaction solution and the mixture was further heated under reflux for 16 hrs. The solvent was evaporated under reduced pressure and the residue was purified by silica gel column chromatography to give the title compound as a colorless oil (3.1 g, yield 99%).

¹H NMR (300 MHz, CDCl₃) δ ppm: 2.21 (s, 6 H), 3.03-3.06 (m, 2 H), 3.25 (s, 2 H), 3.59-3.64 (m, 2 H), 3.95 (br, 1 H), 6.44 (d, J=1.5 Hz, 1 H), 6.53 (dd, J=1.5, 7.5 Hz, 1 H), 6.91 (d, J=7.5 Hz, 1 H).

### Reference Example 273

### ethyl 3-[4-(chloroacetyl)phenyl]propanate

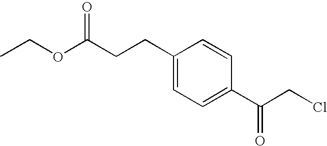

To a solution of ethyl 3-phenylpropanoate (10 g, 56 mmol) and chloroacetyl chloride (9.5 g, 84 mmol) in dichloromethane (50 mL) was slowly added aluminum chloride (22 g, 165 mmol) under ice-cooling, and the mixture was stirred for 3 hrs.

Under ice-cooling, water was slowly added to the reaction solution and the mixture was extracted with ethyl acetate. The extract was washed with saturated brine, dried (MgSO₄), and concentrated under reduced pressure. The residue was purified by column chromatography to give the title compound as pale yellow crystals (14.4 g, yield 99%).

¹H NMR (300 MHz, CDCl₃) δ ppm: 1.22 (t, J=7.2 Hz, 3 H), 2.64 (t, J=7.5 Hz, 2 H), 3.01 (t, J=7.5 Hz, 2 H), 4.11 (q, J=7.2 Hz, 2 H), 4.68 (s, 2 H), 7.32 (d, J=8.4 Hz, 2 H), 7.88 (d, J=8.4 Hz, 2 H).

### Reference Example 274

### 4-(2-carboxyethyl)-3-nitrobenzoic acid

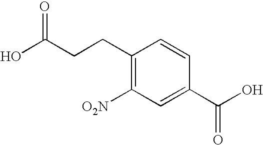

Ethyl 3-[4-(chloroacetyl)phenyl]propanoate (14.4 g, 56 mmol) was dissolved in sulfuric acid (120 mL), and fuming nitric acid (d=1.52) (5.5 mL) was added dropwise under ice-cooling. The mixture was stirred for 2 hrs. Under ice-cooling, water was slowly added to the reaction solution and the mixture was extracted with ethyl acetate. The extract was washed with saturated brine, dried (MgSO₄), and concentrated under reduced pressure. The residue was purified by column chromatography to give the title compound as pale yellow crystals (13.0 g, yield 97%).

¹H NMR (300 MHz, DMSO-d₆) δ ppm: 2.61 (t, J=7.2 Hz, 2 H) 3.12 (t, J=7.2 Hz, 2 H), 7.67 (d, J=8.4 Hz, 1 H), 8.12 (dd, J=1.8, 8.4 Hz, 1 H), 8.36 (d, J=1.8 Hz, 1 H).

### Reference Example 275

### ethyl 4-(3-ethoxy-3-oxopropyl)-3-nitrobenzoate

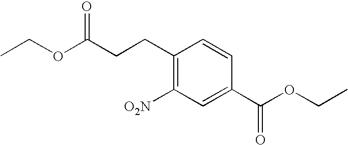

To a solution of 4-(2-carboxyethyl)-3-nitrobenzoic acid (13.0 g, 54 mmol) in ethanol (200 mL) was added sulfuric acid (30 mL) and the mixture was stirred at 80° C. for 1.5 hrs. The reaction solution was cooled to room temperature, and water was added. The mixture was extracted with ethyl acetate. The extract was washed with saturated brine, dried (MgSO₄), and the solvent was evaporated under reduced pressure. The residue was purified by silica gel column chromatography to give the title compound as yellow crystals (14.5 g, yield 90%).

¹H NMR (300 MHz, CDCl₃) δ ppm: 1.22 (t, J=7.2 Hz, 3 H), 1.40 (t, J=7.2 Hz, 3 H), 2.72 (t, J=7.2 Hz, 2 H), 3.26 (t, J=7.2 Hz, 2 H), 4.12 (q, J=7.2 Hz, 2 H), 4.38 (H, q, J=7.2 Hz), 7.50 (1H, d, J=8.1 Hz), 8.17 (dd, J=8.1, 1.8 Hz, 1 H), 8.56 (d, J=1.8 Hz, 1 H).

### Reference Example 276

### ethyl 2-oxo-1,2,3,4-tetrahydroquinoline-7-carboxylate

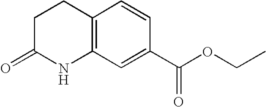

To a solution of ethyl 4-(3-ethoxy-3-oxopropyl)-3-nitrobenzoate (14.5 g, 49 mmol) in ethanol (150 mL) was added 5% palladium-carbon (1.3 g) and the mixture was stirred under a hydrogen atmosphere (1 atm) at 50° C. for 18 hrs. Palladium carbon was collected by filtration, and the filtrate was concentrated. The residue was dissolved in ethanol (100 mL), and triethylamine (7 mL, 50 mmol) was added. The mixture was stirred at 60° C. for 2 hrs. The reaction solution was concentrated under reduced pressure, and the residue was crystallized from diisopropyl ether to give the title compound as white crystals (5.6 g, yield 52%).

¹H NMR (300 MHz, CDCl₃) δ ppm: 1.39 (t, J=7.2 Hz, 3 H), 2.66 (t, J=7.2 Hz, 2 H), 3.02 (t, J=7.2 Hz, 2 H), 4.47 (q, J=5 7.2 Hz, 2 H), 7.23 (d, J=8.1Hz, 1 H), 7.47 (d, J=1.8 Hz, 1 H), 7.67 (dd, J=8.1, 1.8 Hz, 1 H), 8.36 (br, 1 H).

### Reference Example 277

### 2-oxo-1,2,3,4-tetrahydroquinoline-7-carboxylic acid

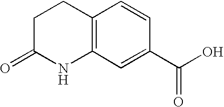

To a solution of ethyl 2-oxo-1,2,3,4-tetrahydroquinoline-7-carboxylate (5.6 g, 25 mmol) in THF (30 mL) was added 1N aqueous sodium hydroxide solution (30 mL) and the mixture was stirred at room temperature for 3 hrs. The reaction solution was acidified by pouring 1N hydrochloric acid thereinto, and the precipitated crystals were collected by filtration. The obtained crystals were washed with water and THF and dried under reduced pressure to give the title compound as white crystals (4.6 g, yield 97%).

¹H NMR (300 MHz, DMSO-d₆) δ ppm: 2.46 (t, J=7.2 Hz, 2 H), 2.92 (t, J=7.2 Hz, 2 H), 7.25 (d, J=7.8 Hz, 1 H), 7.43-7.48 (m, 2 H), 10.21 (s, 1 H), 12.83 (br, 1 H).

### Reference Example 278

### N,N-dimethyl-2-oxo-1,2,3,4-tetrahydroquinoline-7-carboxamide

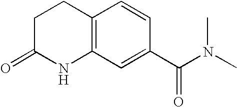

To a solution of 2-oxo-1,2,3,4-tetrahydroquinoline-7-carboxylic acid (4.6 g, 24 mmol) in DMF (50 mL) were added WSC (5.8 g, 30 mmol), HOBt (4.6 g, 30 mmol) and 2.0 M dimethylamine-THF solution (15 mL, 30 mmol), and the mixture was stirred at room temperature for 12 hrs. Saturated aqueous sodium hydrogen carbonate was poured into the reaction solution and the mixture was extracted with ethyl acetate. The extract was washed with 1N hydrochloric acid and saturated brine, dried (MgSO₄) and concentrated under reduced pressure. The residue was crystallized from diisopropyl ether to give the title compound as pale yellow crystals (4.0 g, yield 73%).

¹H NMR (300 MHz, DMSO-d₆) δ ppm: 2.47 (t, J=7.2 Hz, 2 H) 2.85 (t, J=7.2 Hz, 2 H), 2.90 (s, 6 H), 6.84 (s, 1 H), 6.90 (d, J=7.8 Hz, 1 H), 7.18 (d, J=7.8 Hz, 1 H).

### Reference Example 279

### N,N-dimethyl-1-(1,2,3,4-tetrahydroquinolin-7-yl)methanamine dihydrochloride

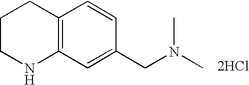

N,N-Dimethyl-2-oxo-1,2,3,4-tetrahydroquinoline-7-carboxamide (4.0 g, 18 mmol) was dissolved in 1.0 M borane-THF solution (200 mL, 200 mmol), and the mixture was heated-under reflux for 12 hrs. Methanol (50 mL) was slowly added to the reaction solution and the mixture was further heated under reflux for 16 hrs. The solvent was evaporated under reduced pressure, and the residue was purified by silica gel column chromatography and crystallized by adding 4N hydrochloric acid-ethyl acetate solution to give the title compound as white crystals (3.67 g, yield 77%).

¹H NMR (300 MHz, DMSO-d₆) δ ppm: 1.86-1.91 (m, 2 H), 2.64 (s, 6 H), 2.72-2.77 (m, 2 H), 3.22-3.26 (m, 2 H), 4.14-4.17 (m, 2 H), 7.00-7.15 (m, 3 H), 10.86 (br, 1 H).

### Reference Example 280

### 1-(3-nitrophenyl)ethyl methanesulfonate

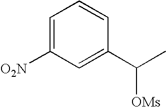

To a solution of 3-nitroacetophenone (2.4 g, 15 mmol) in ethanol (50 mL) was added sodium borohydride (0.8 g, 20 mmol) and the mixture was stirred at room temperature for 30 min. 1N Hydrochloric acid was poured into the reaction solution and the mixture was extracted with ethyl acetate. The extract was washed with saturated brine, dried (MgSO₄) and concentrated under reduced pressure. The residue was dissolved in DMF (30 mL), and triethylamine (2.8 mL, 20 mmol) and methanesulfonyl chloride (1.6 mL, 20 mmol) were added. The mixture was stirred at room temperature for 4 hrs. Water was poured into the reaction solution and the mixture was extracted with ethyl acetate. The extract was washed with saturated brine, dried (MgSO₄) and concentrated under reduced pressure. The residue was purified by silica gel column chromatography to give the title compound as a colorless oil (3.1 g, yield 83%).

¹H NMR (300 MHz, CDCl₃) δ ppm: 1.75 (d, J=6.6 Hz, 3 H), 2.94 (s, 3 H), 2.72-2.77 (m, 2 H), 5.83 (q, J=6.6 Hz, 1 H), 7.57-7.62 (m, 1 H), 7.72-7.76 (m, 1 H), 8.18-8.27 (m, 2 H).

### Reference Example 281

### N,N-dimethyl-1-(3-nitrophenyl)ethaneamine

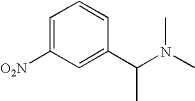

To a solution of 1-(3-nitrophenyl)ethyl methanesulfonate (3.0 g, 12 mmol) in THF (5 mL) was added 2.0 M dimethylamine-THF solution (10 mL, 20 mmol) and the mixture was stirred at 50° C. for 16 hrs. The reaction solution was concentrated under reduced pressure. The residue was purified by column chromatography to give the title compound as a colorless oil (1.5 g, yield 66%).

¹H NMR (300 MHz, CDCl₃) δ ppm: 1.34 (d, J=6.6 Hz, 3 H), 2.16 (s, 3 H), 3.34 (q, J=6.6 Hz, 1 H), 7.39-7.46 (m, 1 H), 7.60-7.65 .(m, 1 H), 8.03-8.08 (m, 1 H), 8.21-8.25 (m, 1 H).

The compound described in the following Reference Example 282 was produced in the similar manner as in Reference Example 10.

### Reference Example 282

### [1-(3-aminophenyl)ethyl]dimethylamine

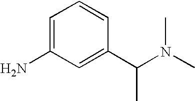

¹H NMR (300 MHz, CDCl₃) δ ppm: 1.34 (d, J=6.6 Hz, 3 H), 2.20 (s, 3 H), 3.11 (q, J=6.6 Hz, 1 H), 3.63 (br, 2 H), 6.55-6.58 (m, 1 H), 6.65-6.68 (m, 2 H), 7.05-7.11 (m, 1 H).

### Reference Example 283

### 4-(chloromethyl)-1,3-thiazole-2-amine hydrochloride

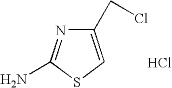

To a solution of 1,3-dichloroacetone (25 g, 200 mmol) in acetone (100 mL) was added dropwise a solution (500 mL) of thiourea (15 g, 200 mmol) in acetone and the mixture was stirred at room temperature for 2 hrs. The reaction solution was concentrated under reduced pressure, and ethanol (200 mL) was added. The mixture was stirred for 2 hrs. and the precipitated crystals were removed. Hexane and diisopropyl ether were added to the filtrate. The precipitated crystals were collected by filtration and vacuum dried to give the title compound as white crystals (16.4 g, yield 45%).

¹H NMR (300 MHz, DMSO-d₆) δ ppm: 4.66 (s, 2 H), 6.96 (s, 1 H), 9.29 (br, 3 H).

### Reference Example 284

### 4-[(dimethylamino)methyl]-1,3-thiazole-2-amine

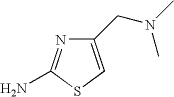

4-(Chloromethyl)-1,3-thiazole-2-amine hydrochloride (8.2 g, 37 mmol) was dissolved in water (5 mL), and an aqueous solution of 50% dimethylamine (50 mL) was added dropwise under ice-cooling. The mixture was stirred for 1 hr. and water was poured into the reaction solution. The mixture was extracted with ethyl acetate, and the extract was washed with saturated brine, dried (MgSO₄) and concentrated under reduced pressure. The residue was purified by silica gel column chromatography to give the title compound as white crystals (3.05 g, yield 53%).

¹H NMR (300 MHz, CDCl₃) δ ppm: 2.26 (s, 6 H), 3.31 (s, 2 H), 4.91 (br, 2 H), 6.30 (s, 1 H).

### Reference Example 285

### [4-(cyclopropylmethoxy)-3-nitrobenzyl]dimethylamine

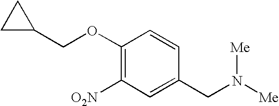

A solution of 4-hydroxy-3-nitrobenzaldehyde (10.0 g, 59.8 mmol), cyclopropylmethylbromide (6.96 mL, 71.8 mmol) and potassium carbonate (16.5 g, 120 mmol) in acetonitrile (150 mL)—dimethylformamide (150 mL) was stirred at 60° C. for 16 hrs. The reaction solution was concentrated under reduced pressure and the residue was dissolved in ethyl acetate. This solution was washed successively with aqueous potassium carbonate solution, and then saturated brine and dried (Na₂SO₄). The solvent was concentrated under reduced pressure and the obtained residue was purified by alumina column chromatography (developing solvent; ethyl acetate). To a solution of the obtained oil (8.54 g, 38.6 mmol) and 2.0 M dimethylamine-THF solution (23.2 mL) in THF (100 mL) was added triacetoxysodium borohydride (9.82 g, 46.3 mmol) at 0° C. and the mixture was stirred at room temperature for 2 hrs. The reaction solution was concentrated under reduced pressure, and the residue was dissolved in 1N hydrochloric acid and washed with diethyl ether. Potassium carbonate was added to basify the aqueous layer and the mixture was extracted with ethyl acetate. The organic layer was washed with saturated brine, and dried (Na₂SO₄). The solvent was concentrated under reduced pressure, and the obtained residue was purified by aminopropyl silica gel column chromatography (developing solvent; ethyl acetate) to give the title compound (8.52 g).

¹H NMR (300 MHz, CDCl₃) δ ppm: 0.36-0.41 (m, 2 H), 0.62-0.68 (m, 2 H), 1.24-1.31 (m, 1 H), 2.23 (s, 6 H), 3.38 (s, 2 H), 3.96 (d, J=6.6 Hz, 2 H), 7.01 (d, J=8.7 Hz, 1 H), 7.44 (dd, J=8.1, 2.1 Hz, 1 H), 6.69 (d, J=2.1 Hz, 1 H).

### Reference Example 286

### [3-amino-4-(cyclopropylmethoxy)benzyl]dimethylamine

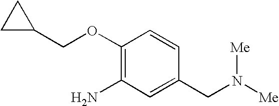

To a solution of [4-(cyclopropylmethoxy)-3-nitrobenzyl]dimethylamine (8.50 g, 34.0 mmol) and 10% palladium-carbon (850 mg) in ethanol (34 mL) was added dropwise hydrazine monohydrate (4.94 mL, 102 mmol) at room temperature and the mixture was stirred at 80° C. for 2 hrs. Palladium-carbon was filtered, and the solvent was evaporated under reduced pressure. The residue was dissolved in ethyl acetate, washed with saturated brine and dried (Na₂SO₄). The solvent was concentrated under reduced pressure and the obtained residue was purified by aminopropyl silica gel column chromatography (developing solvent: hexane/ethyl acetate=17/3) to give the title compound (4.86 g).

¹H NMR (300 MHz, CDCl₃) δ ppm: 0.31-0.36 (m, 2 H), 0.58-0.65 (m, 2 H), 1.24-1.28 (m, 1 H), 2.21 (s, 6 H), 3.28 (s, -2 H), 3.80-3.82 (m, 4 H), 6.59 (d, J=8.1 Hz, 1 H), 6.67-6.71 (m, 2 H).

### Reference Example 287

### 1-[4-(hydroxymethyl)-2-nitrophenyl]ethanone

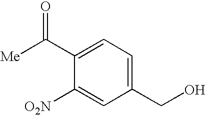

To a solution of 4-fluoro-3-nitrobenzyl alcohol (10.1 g, 59.2 mmol) and nitroethane (18.6 mL, 245 mmol) in ethyl acetate (120 mL) was added DBU (25.7 mL, 177 mmol) and the mixture was stirred at room temperature for 16 hrs. The reaction solution was concentrated and dissolved in methanol (100 mL). 30% Aqueous hydrogen peroxide (30 mL) and 10% aqueous sodium hydrogen carbonate (30 mL) were added and the mixture was stirred at room temperature for 16 hrs. The reaction solution was concentrated under reduced pressure, and the residue was dissolved in ethyl acetate. This solution was washed with 1N hydrochloric acid, washed with saturated brine and dried (Na₂SO₄). The solvent was concentrated under reduced pressure and the obtained residue was purified by silica gel column chromatography (developing solvent: hexane/ethyl acetate=3/1-2/1) to give the title compound (3.74 g).

¹H NMR (300 MHz, CDCl₃) δ ppm: 2.05-2.07 (m, 1 H), 2.55 (s, 3 H), 4.84 (d, J=5.4 Hz, 2 H), 7.43 (d, J=7.8 Hz, 1 H), 7.69 (d, J=7.8 Hz, 1 H)₁ 8.09 (s, 1 H).

### Reference Example 288

### 1-[2-amino-4-(hydroxymethyl)phenyl]ethanone

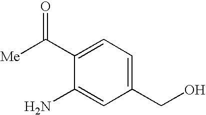

A solution of 1-[4-(hydroxymethyl)-2-nitrophenyl]ethanone (3.74 g, 19.2 mmol), ammonium formate (6.04 g, 95.8 mmol) and 10% palladium-carbon (1.2 g) in methanol (70 mL) was stirred at 60° C. for 6 hrs and the mixture was stirred at room temperature for 6 hrs. Palladium-carbon was filtered and the solvent was evaporated under reduced pressure. The residue was dissolved in ethyl acetate and washed with aqueous potassium carbonate solution and then washed with saturated brine, and dried (Na₂SO₄). The solvent was concentrated under reduced pressure and the obtained residue was purified by silica gel column chromatography (developing solvent: hexane/ethyl acetate=1/1) and powdered with diisopropyl ether and hexane to give the title compound (1.75 g).

¹H NMR (300 MHz, CDCl₃) δ ppm: 1.91 (t, J=6.0 Hz, 1 H), 2.56 (s, 3 H), 4.63 (d, J=6.0 Hz, 2 H), 6.31 (br, 2 H), 6.61 (d, J=8.4 Hz, 1 H), 6.66 (s, 1 H), 7.69 (d, J=8.4 Hz, 1 H).

### Reference Example 289

### 1-[2-amino-4-(chloromethyl)-phenyl]ethanone hydrochloride

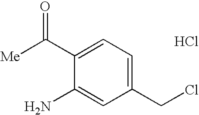

1-[2-Amino-4-(hydroxymethyl)phenyl]ethanone (1.54 g, 9.32 mmol) was added to thionyl chloride (30 mL) at 0° C. and the mixture was stirred at room temperature for 3 hrs. The reaction solution was concentrated under reduced pressure, and the obtained residue was powdered with diisopropyl ether to give the title compound (1.45 g).

¹H NMR (300 MHz, DMSO-d₆) δ ppm: 2.50 (s, 3 H), 4.64 (s, 2 H), 6.59 (dd, J=1.8, 8.1 Hz, 1 H), 6.81 (d, J=1.8 Hz, 1 H), 7.74 (d, J=8.1 Hz, 1 H).

### Reference Example 290

### 1-{2-amino-4-[(dimethylamino)methyl]phenyl}ethanone

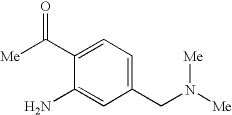

To a solution of 1-[2-amino-4-(chloromethyl)phenyl]ethanone hydrochloride (1.45 g, 6.59 mmol) in chloroform (15 mL) was added 50% aqueous dimethyl amine solution (1.78 mL, 19.8 mmol) and the mixture was stirred at room temperature for 16 hrs. The reaction solution was concentrated under reduced pressure and the residue was dissolved in ethyl acetate. This solution was washed with aqueous potassium carbonate solution and then washed with saturated brine, and dried (Na₂SO₄). The solvent was concentrated under reduced pressure and the obtained residue was purified by silica gel column chromatography (developing solvent: ethyl acetate/methanol=2/1) and purified by aminopropyl silica gel column chromatography (developing solvent: hexane/ethyl acetate=3/1) to give the title compound (424 mg).

¹H NMR (300 MHz, CDCl₃) δ ppm: 2.24 (s, 6 H), 2.56 (s, 3 H), 3.32 (s, 2 H), 6.27 (br, 2 Hr), 6.59 (d, J=9.0 Hz, 1 H), 6.62 (s, 1 H), 7.66 (d, J=9.0 Hz, 1 H)

### Reference Example 291

### N-methoxy-N-methyl-4-phenylcyclohexanecarboxamide

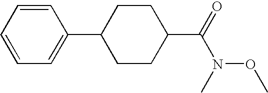

4-Phenylcyclohexylcarboxylic acid (2.3 g, 10 mmol) and N,O-dimethylhydroxylamine hydrochloride (1.07 g, 11 mmol) were suspended in DMF (30 mL) and cooled to 0° C. Thereto were successively added diethyl cyanophosphate (1.7 mL, 1.1 mmol) and triethylamine (3.1 mL, 2.2 mmol), and the mixture was stirred at the same temperature for 1 hr and at room temperature for 3 hrs. To the reaction solution was added aqueous 1M-sulfuric acid hydrogen potassium solution and the mixture was extracted with ethyl acetate. The extract was washed with brine, dried (MgSO₄) and concentrated. The residue was subjected to silica gel column chromatography to give the title compound as an amorphous powder (1.85 g, yield 68%).

¹H NMR (400 MHz, CDCl₃) δ ppm: 1.47-1.77 (m, 4 H), 1.89-2.03 (m, 4 H), 2.58 (tt, J=12.1, 3.3 Hz, 1 H), 2.70-2.84 (m, 1 H), 3.21 (s, 3 H), 3.73-3.74 (m, 3 H), 7.12-7.40 (m, 5 H).

LC/MS (ESI) m/z: 248 (M+H⁺).

### Example 292

### 4-phenylcyclohexanecarbaldehyde

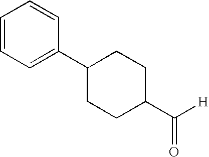

To a solution of N-methoxy-N-methyl-4-phenylcyclohexanecarboxamide (190 mg, 0.77 mmol) in THF (5 mL) cooled to 0° C. was added aluminum lithium hydride (37 mg, 0.96 mmol) and the mixture was stirred at 0° C. for 2 hrs. To the reaction solution was added saturated aqueous ammonium chloride solution and insoluble materials were removed by decantation. The organic layer was concentrated and the residue was purified by silica gel column chromatography (developing solvent: hexane/ethyl acetate 4/1) to give the title compound as an oil (130 mg, yield 90%).

¹H NMR (.400 MHz, CDCl₃) δ ppm: 1.27-1.58 (m, 4 H), 2.02-2.15 (m, 4 H), 2.23-2.41 (m, 1 H), 2.50 (tt, J=12.1, 3.3 Hz, 1 H), 7.16-7.33 (m, 5 H), 9.68 (s, 1 H).

### Reference Example 293

### ethyl 1-phenylpiperidine-4-carboxylate

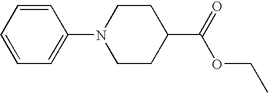

To a solution of tetrakisdibenzylidene acetone dipalladium (Pd₂(dba)₃) (173 mg, 0.36 mmol), BINAP (560 mg, 0.9 mmol) and sodium tert-butoxide (1.3 g, 13.5 mmol) in toluene (dry, 20 mL) were added bromobenzene (1.6 g, 10 mmol) and ethyl isonipecotinate (1.7 g, 11 mmol) and the mixture was stirred at 85° C. for 1.5 hrs. To the reaction solution was added diethyl ether (20 mL) and insoluble materials were celite filtered. The mother liquor was concentrated. The residue was purified by silica gel column chromatography to give the title compound-as a slightly yellow oil (1.7 g, yield 72%).

¹H NMR (400 MHz, CDCl₃) δ ppm: 1.27 (t, J=7.1 Hz, 3 H), 1.82-1.93 (m, 2 H), 2.00-2.05 (m, 2 H), 2.36-2.47 (tt, J=10.9, 3.9 Hz, 1 H), 2.78 (tt, J=12.1, 2.2 Hz, 2 H), 3.62-3.67 (m, 2 H), 4.16 (q, J=7.1 Hz, 2 H), 6.82-6.95 (m, 3 H), 7.23-7.28 (m, 2 H).

LC/MS (ESI) m/z 234 (M+H⁺).

### Reference Example 294

### (1-phenyl-piperidin-4-yl)-methanol

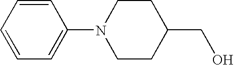

To a solution of ethyl 1-phenylpiperidine-4-carboxylate (2.6 g, 6.86 mmol) in THF. (32 mL) cooled to 0° C. was added aluminum lithium hydride (325 mg, 8.58 mmol) and the mixture was stirred at 0° C. for 2 hrs. To the reaction solution was added saturated aqueous ammonium chloride solution and insoluble materials were removed by decantation. The organic layer was dried over anhydrous magnesium sulfate and concentrated. The residue was purified by silica gel column chromatography (developing solvent: hexane/ethyl acetate=4/1) to give the title compound as a colorless solid (1.3 g, 99%).

¹H NMR (400 MHz, CDCl₃) δ ppm: 1.25-1.46 (m, 3 H), 1.62-1.69 (m, 1 H), 1.83-1.88 (m, 2 H), 2.72 (td, J=12.2, 2.4 Hz, 1 H), 3.55 (t, J=5.8 Hz, 2 H), 3.69-3.74 (m, 2 H), 6.81-6.96 (m, 3 H), 7.23-7.28 (m, 2 H).

### Reference Example 295

### 1-phenylpiperidine-4-carbaldehyde

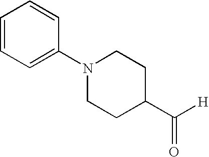

(1-Phenyl-piperidin-4-yl)-methanol (0.81 g, 4.23 mmol) and DBU (1.29 g, 8.46 mmol) were dissolved in dehydrating dichloromethane (17 mL) and the mixture was cooled to −78° C. Thereto was added a solution of N-tert-butylphenylsulfinimidoyl chloride (1.0 g/) in dichloromethane (5 mL) and the mixture was stirred while maintaining −60° C. or below for 1 hr. Water was added to the reaction solution and the mixture was extracted with diethyl ether. The extract was dried (MgSO₄) and concentrated. The residue was purified by silica gel column chromatography (developing solvent: hexane/ethyl acetate=4/1) to give the title compound (720 mg, yield 90%).

¹H NMR (400 MHz, CDCl₃) δ ppm: 1.75-1.85 (m, 2 H), 2.00-2.06 (m, 2 H), 2.36-2.46 (m, 1 H), 2.83-2.90 (m, 2 H), 3.61 (t, J=3.8 Hz, 1 H), 3.64 (t, J=3.8 Hz, 1 H), 6.84-6.96 (m, 3 H), 7.23-7.29 (m, 2 H), 9.71 (s, 1 H).

The compound described in the following Reference Example 296 was produced in the similar manner as in Reference Example 24.

### Reference Example 296

### ethyl (2R,3S)-3-(1H-indol-3-yl)-2-{[(3-oxo-4-phenylpiperazin-1-yl)carbonyl]amino}butanoate

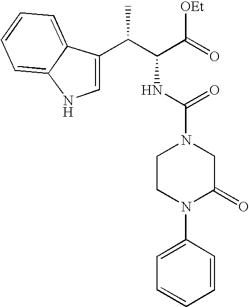

¹H NMR (400 MHz, CDCl₃) δ ppm: 1.16 (t, J=7.2 Hz, 2 H), 1.50 (d, J=7.3 Hz, 3 H), 3.60-3.81 (m, 5 H), 3.96-4.19 (m, 2 H), 4.07-4.16 (m, 2 H), 4.81 (dd, J=8.1, 5.4 Hz, 1 H), 4.92 (d, J=8.1 Hz, 1 H), 7.01-7.46 (m, 9 H), 7.63 (d, J=8.1 Hz, 1 H), 8.11 (s, 1 H).

The compound described in the following Reference Example 297 was produced in the similar manner as in Reference Example 2.

### Reference Example 297

### (2R,3S)-2-({[4-(tert-butoxycarbonyl)piperazin-1-yl]carbonyl}amino)-3-(1H-indol-3-yl)butanoic acid

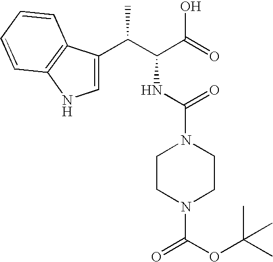

¹H NMR (400 MHz, CDCl₃) δ ppm: 1.46 (s, 9 H), 1.53 (d, J=7.3 Hz, 3 H), 3.16-3.44 (m, 8 H), 3.67-3.74 (m, 1 H), 4.70 (dd, J=7.6, 5.3 Hz, 1 H), 5.04 (d, J=7.6 Hz, 1 H), 7.05-7.33 (m, 4 H), 7.60 (d, J=7.8 Hz, 1 H), 8.50 (s, 1 H), 10.10 (brs, 1,H).

### Reference Example 298

### 2-[(2-hydroxyethyl)amino]-N-phenylacetamide

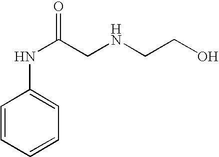

To a mixed solution of aniline (9.8 g, 104 mmol), 20% aqueous solution of potassium hydrogen carbonate (150 mL) and ethyl acetate (150 mL), which had been cooled to 0° C., was added dropwise chloroacetyl chloride (14 g, 126 mmol) over 30 min. The mixture was stirred for 10 min and the organic layer was separated and dried (MgSO₄). Thereto was added 2-hydroxyethylamine (21.5 g, 352 mmol) and the mixture was stirred at 60° C. for 2 hrs. The reaction solution was ice-cooled and the precipitated crystals were washed with water and vacuum dried to give the title compound as colorless crystals (14.5 g, yield 72%).

¹H NMR (400 MHz, CDCl₃) δ ppm: 2.12 (s, 2 H), 2.82-2.86 (m, 2 H), 3.41 (s, 2 H), 3.72-3.77 (m, 1 H), 7.08-7.12 (m, 1H), 7.28-7.36 (m, 2 H), 7.47-7.68 (m, 2 H), 9.34 (s, 1 H).

### Reference Example 299

### 1-phenylpiperazin-2-one

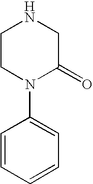

A mixture of 2-[(2-hydroxyethyl)amino]-N-phenylacetamide (1.94 g, 10 mmol) and tributylphosphine (3.5 mL) in ethyl acetate (20 mL) was cooled to 0° C. and 1,1′-(azocarbonyl)dipiperidine (3.2 g, 12.5 mmol) was added. The mixture was stirred at the same temperature for 30 min, then heated to 40° C. and stirred for 2 hrs. The reaction solution was filtered, and the filtrate was concentrated. The residue was subjected to silica gel column chromatography (developing solvent: ethyl acetate/ethanol=20/1) to give the title compound as colorless crystals (550 mg, yield 31%).

¹H NMR (400 MHz, CDCl₃) δ ppm: 3.20-3.25 (m, 2 H), 3.37-3.42 (m, 2 H), 3.67-3.72 (m, 3 H), 7.14-7.60 (m, 5 H).

### Reference Example 300

### (2R,3S)-2-({ [(2-anilinoethyl)(carboxymethyl)amino]carbonyl}amino)-3-(1H-indol-3-yl)butanoic acid

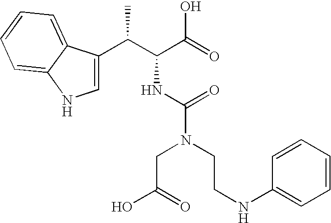

Ethyl (2R,3S)-3-(1H-indol-3-yl)-2-{[(3-oxo-4-phenylpiperazin-1-yl)carbonyl]amino}butanoate (300 mg, 0.67 mmol) was dissolved in ethanol (8 mL), and 2N aqueous sodium hydroxide solution (4 mL) was added. The mixture was stirred at 40° C. for 2 hrs. The reaction solution was concentrated, and 10% aqueous citric acid solution (10 mL) was added to the residue. The precipitated crystals: were collected by filtration. After washing with water, the crystals were dried under reduced pressure to give the title compound as colorless crystals (160 mg, yield 55%).

¹H NMR (400 MHz, DMSO-d₆) δ ppm: 1.27 (d, J=7.1 Hz, 3 H) 2.45-2.57 (m, 3 H), 3.14 (t, J=6.5 Hz, 2 H), 3.25-3.55 (m, 2 H), 3.91-4.06 (m, 1 H), 4.44 (d, J=8.1 Hz, 1 H), 6.27 (d, J=8.6 Hz, 1 H), 6.46-6.62 (m, 3 H)-, 6.87-7.16 (m, 5 H), 7.32 (d, J=7.8 Hz, 1 H), 7.52 (d, J=7.8 Hz, 1 H), 10.82 (d, J=1.7 Hz, 1 H), 12.3 (brs, 1H).

LC/MS (ESI) m/z 439 (M+H⁺).

### Reference Example 301

### (2R,3S)-3-(1H-indol-3-yl)-2-{[(2-phenylpyrrolidin-1-yl)carbonyl]amino}butanoic acid

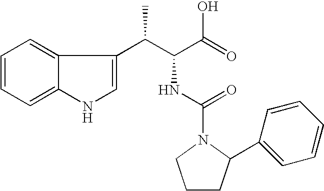

To a solution of ethyl (2R,3S)-2-amino-3-(1H-indol-3-yl)butanoate methanesulfonate (500 mg) and triethylamine (0.233 mL) in acetonitrile (8 mL) was added N,N′-disuccinimidyl carbonate (0.429 mg) under ice-cooling and the mixture was stirred for 1 hr. To the obtained solution was added a solution of 2-phenylpyrrolidine (247 mg) in acetonitrile (1 mL) under ice-cooling. The reaction solution as stirred at room temperature for 16 hrs, and 1N hydrochloric acid was added. The mixture was extracted with ethyl acetate. The extract was washed with saturated potassium carbonate solution and saturated brine and dried (Na₂SO₄). The solvent was evaporated under reduced pressure and the residue was purified by aminopropyl silica gel column chromatography (developing solvent: ethyl acetate) (546 mg, yield 89%). To a solution of the obtained methyl (2R,3S)-3-(1H-indol-3-yl)-2-{[(2-phenylpyrrolidin-1-yl)carbonyl]amino}butanoate (546 mg) in methanol (11 mL) was added 2N aqueous sodium hydroxide (2.02 mL) at room temperature and the mixture was stirred for 16 hrs. To the reaction solution was added 1N hydrochloric acid and the mixture was extracted with ethyl acetate. The extract was dried (Na₂SO₄) and the solvent was evaporated under reduced pressure to give the title compound as an amorphous powder (436 mg, yield 83%).

LC/MS (ESI) m/z 392 (M+H⁺)

The compound described in the following Reference Example 302 was produced in the similar manner as in Reference Example 301.

### Reference Example 302

### (2R,3S)-3-(1H-indol-3-yl)-2-{[(2-benzylpyrrolidin-1-yl)carbonyl]amino}butanoic acid

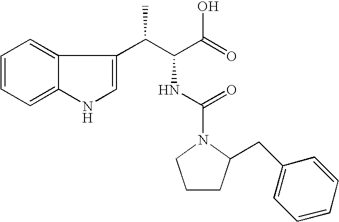

LC/MS (ESI) m/z 406 (M+H⁺).

### Reference Example 303

### N-(3-aminobenzyl)-2,2,2-trifluoroacetamide

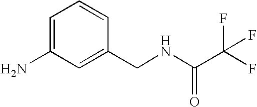

3-(Aminomethyl)aniline (2.0 g, 16 mmol) was dissolved in methanol (5.0 mL) and ethyl trifluoroacetate (1.8 mL, 15 mmol) as added at room temperature. The mixture was stirred overnight. The reaction solution was concentrated, and the residue was dissolved in ethyl acetate and filtered through an alumina layer. The mother liquor was concentrated, and vacuum dried to give the title compound (3.4 g, quant.).

¹H NMR (300 MHz, CDCl₃) δ ppm: 3.74 (s, 2 H), 4.43 (d, J=5.7 Hz, 2 H), 6.52-6.57 (m, 1 H), 6.58-6.61 (m, 1 H), 6.63-6.72 (m, 2 H), 7.15 (t, J=7.8 Hz, 1 H).

The compounds described in the following Reference Examples 304-305 were produced in the similar manner as in Reference Example 303.

### Reference Example 304

### N-(2-aminobenzyl)-2,2,2-trifluoroacetamide

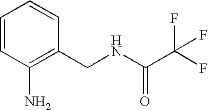

¹H NMR (300 MHz, CDCl₃) δ ppm: 4.02 (s, 2 H), 4.47 (d, J=6.2 Hz, 2 H), 6.64-6.79 (m, 3 H), 7.09 (dd, J=7.5, 1.5 Hz, 1 H), 7.17 (td, J=7.7, 1.6 Hz, 1 H).

### Reference Example 305

### N-(4-aminobenzyl)-2,2,2-trifluoroacetamide

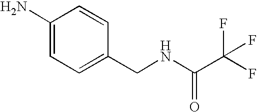

¹H NMR (300 MHz, CDCl₃) δ ppm: 3.74 (s, 1 H), 4.39 (d, J=5.6 Hz, 2 H), 6.57 (s, 1 H), 6.66 (d, J=8.6 Hz, 2 H), 7.08 (d, J=8.6 Hz, 2 H).

### Reference Example 306

### tert-butyl (3-aminobenzyl)carbamate

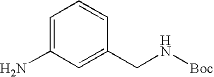

3-(Aminomethyl)aniline (1.3 g, 11 mmol) was dissolved in acetonitrile (3.0 mL) and a solution (3.0 mL) of tert-butyl dicarboxylate (2.2 g, 10 mmol) in acetonitrile was slowly added dropwise at room temperature. The reaction solution was stirred overnight, diluted with ethyl acetate, and filtered through a silica gel layer. The mother liquor was concentrated and vacuum dried to give the title compound (2.3 g, quant.).

¹H NMR (300 MHz, CDCl₃) δ ppm: 1. 46 (s, 9 H), 3. 67 (s, 2 H), 4.22 (d, J=5.9 Hz, 2 H), 4.82 (s, 1 H), 6.56-6.63 (m, 2 H) 6.66 (d, J=7.6 Hz, 1 H), 7.11 (t, J=7.9 Hz, 1 H).

### Reference Example 307

### tert-butyl (4-aminobenzyl)carbamate

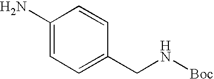

4-(Aminomethyl)aniline (1.3 g, 11 mmol) was dissolved in acetonitrile (3.0 mL) and a solution (3.0 mL) of di-tert-butyl dicarbonate (2.2 g, 10 mmol) in acetonitrile was slowly added dropwise at room temperature. The reaction solution was stirred overnight, diluted with ethyl acetate, and filtered through a silica gel layer. The mother liquor was concentrated, and the residue was solidified with hexane and ethyl acetate. The obtained solid powder was collected by filtration, washed with hexane and dried under reduced pressure to give the title compound.

¹H NMR (300 MHz, CDCl₃) δ ppm: 1. 45 (s, 9 H), 3. 65 (s, 2 H), 4.19 (d, J=5.6 Hz, 2 H), 4.72 (s, 1 H), 6.64 (d, J=8.5 Hz, 2 H), 7.07 (d, J=8.2 Hz, 2 H).

The compound described in the following Reference Example 308 was produced in the similar manner as in Reference Example 307.

### Reference Example 308

### tert-butyl (2-aminobenzyl)carbamate

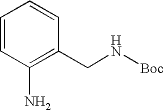

¹H NMR (300 MHz, CDCl₃) δ ppm: 1.45 (s, 9 H), 4.15-4.26 (br, 2 H), 4.25 (d, J=6.2 Hz, 2 H), 4.78 (s, 1 H), 6.63-6.71 (m, 2 H), 7.03 (dd, J=7.7, 1.4 Hz, 1 H), 7.10 (td, J=7.7, 1.5 Hz, 1 H).

### Reference Example 309

### tert-butyl [4-(methylamino)benzyl]carbamate

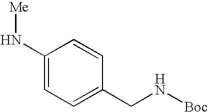

tert-Butyl (4-aminobenzyl)carbamate (0.92 g, 4.2 mmol) and benzotriazole (0.50 g, 4.2 mmol) were dissolved in ethanol (4.0 mL) and aqueous solution of formaldehyde (37%, 0.30 mL, 4.0 mmol) was added at room temperature. The mixture was stirred overnight and the solvent was evaporated. The residue was dissolved in THF (4.0 mL) and sodium borohydride (0.18 g, 4.7 mmol) was added at room temperature. The mixture was stirred for 4 hrs. Sodium borohydride (0.065 g, 1.8 mmol) was further added and the mixture was stirred for 3 hrs. Then, saturated aqueous solution of sodium hydrogen carbonate and ethyl acetate were added and the mixture was subjected to extraction. The organic layer was dried (Na₂SO₄) and concentrated under reduced pressure. The residue was purified by silica gel chromatography to give the title compound (0.42 g, yield 44%).

¹H NMR (300 MHz, CDCl₃) δ ppm: 1.46 (s, 9 H), 2.83 (s, 3 H), 3.71 (s, 1 H), 4.19 (d, J=5.7 Hz, 2 H), 4.70 (s, 1 H), 6.55-6.60 (m, 2 H), 7.11 (d, J=8.4 Hz, 2 H).

### Reference Example 310

### tert-butyl (3-(methylamino)benzyl]carbamate

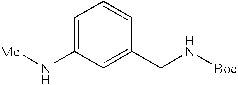

tert-Butyl (3-aminobenzyl)carbamate (0.92 g, 4.2 mmol) and benzotriazole (0.50 g, 4.2 mmol) were dissolved in ethanol (4.0 mL) and aqueous solution of formaldehyde (37%, 0.30 mL, 4.0 mmol) was added at room temperature. The mixture was stirred overnight and the solvent was evaporated. The residue was dissolved in THF (4.0 mL) and sodium borohydride (0.18 g, 4.7 mmol) was added at room temperature. The mixture was stirred for 4 hrs. Sodium borohydride (0.065 g, 1.8 mmol) was further added and the mixture was stirred for 3 hrs. Saturated aqueous sodium hydrogen carbonate and ethyl acetate were added and the mixture was subjected to extraction. The organic layer was dried (Na₂SO₄) and concentrated under reduced pressure. The residue was purified by silica gel chromatography to give the title compound (0.42 g, yield 44%).

¹H NMR (300 MHz, CDCl₃) δ ppm: 1.46 (s, 9 H), 2.83 (s, 3 H), 3.70-3.77 (m, 1 H), 4.24 (d, J=5.7 Hz, 2 H), 4.76.-4.84 (m, 1 H), 6.49-6.55 (m, 2 H), 6.62 (d, J=7.6 Hz, 1 H), 7.11-7.18 (m, 1 H).

### Reference Example 311

### 2,2,2-trifluoro-N-[3-(methylamino)benzyl]acetamide

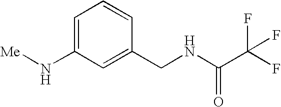

4N Hydrochloric acid-dioxane solution (4.0 mL) was added to tert-butyl [3-(methylamino)benzyl]carbamate (0.22 g, 0.91 mmol) at room temperature and the mixture was stirred overnight. The reaction suspension was concentrated under reduced pressure, and the residue was dissolved in methanol (2.0 mL). DBU (0.27 mL, 1.81 mmol) and ethyl trifluoroacetate (0.11 mL, 0.92 mmol) were added to this solution at room temperature and the mixture was stirred for 3 hrs. The reaction solution was concentrated, diluted with ethyl acetate and filtered through a silica gel layer. The mother liquor was concentrated to give the title compound (0.21 g, yield 99%).

¹H NMR (300 MHz, CDCl₃) δ ppm: 2.84 (s, 3 H), 4.45 (d, J=5.7 Hz, 2 H), 6.49-6.53 (m, 1 H), 6.53.-6.64 (m, 3 H), 7.18 (t, J=7.8 Hz, 1 H).

The compound described in the following Reference Example 312 was produced in the similar manner as in Reference Example 311.

### Reference Example 312

### 2,2,2-trifluoro-N-[4-(methylamino)benzyl]acetamide

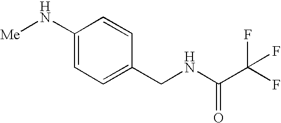

¹H NMR (300 MHz, CDCl₃) δ ppm: 2.84 (s, 3 H), 4.41 (d, J=5.4 Hz, 2 H), 6.59 (d, J=8.6 Hz, 2 H), 7.12 (d, J=8.6 Hz, 2 H).

### Reference Example 313

### N-[2-(3-aminophenyl)ethyl]-2,2,2-trifluoroacetamide

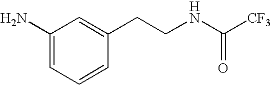

(3-Nitrophenyl)acetonitrile (1.6 g, 10 mmol) was dissolved in ethanol (10 mL) and 10% palladium-carbon (0.17 g) was added. To the reaction suspension was added dropwise hydrazine monohydrate (1.5 g, 30 mmol) at room temperature at a rate of allowing reflux of the solvent due to the reaction heat and the mixture was stirred at room temperature for 5 hrs. Palladium-carbon was filtered off and the reaction solution was concentrated. The residue was dissolved in ethanol (10 mL) and Raney-nickel was added. To the suspension was slowly added dropwise hydrazine monohydrate (2.0 g, 4.0 mmol) at 50° C. and the mixture was stirred at 50° C. for 4 hrs. Hydrazine monohydrate (2.0 g, 4.0 mmol) was slowly added dropwise further at 50° C. and Raney-nickel was added. The mixture was stirred overnight at 50° C. Raney-nickel was removed by celite filtration and the mother liquor was concentrated. The residue was dissolved in methanol (10 mL). To this solution was added ethyl trifluoroacetate (1.3 mL, 0.92 mmol) at room temperature and the mixture was stirred for 2 days. The reaction solution was concentrated and the residue was purified by silica gel column chromatography to give the title compound (1.3 g, yield 54%).

¹H NMR (300 MHz, CDCl₃) δ ppm: 2.78 (t, J=6.8 Hz, 2. H), 3.59 (q, J=6.8 Hz, 2 H), 3.69 (brs, 2 H), 6.32 (brs, 1 H), 6.47-6.51 (m, 1 H), 6.53-6.61 (m, 2 H), 7.11 (t, J=7.7 Hz, 1 H)

### Reference Example 314

### 1-(3-nitrobenzyl)pyrrolidine

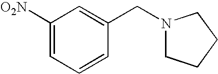

1-(Chloromethyl)-3-nitrobenzene (1.0 g, 5.9 mmol) was dissolved in THF (5.0 mL), and pyrrolidine (0.58 mL, 6.95 mmol) was added at room temperature. The mixture was stirred overnight. To the reaction solution were added saturated aqueous solution of sodium hydrogen carbonate and ethyl acetate and the mixture was subjected to extraction. The organic layer was dried (Na₂SO₄) and concentrated. The residue was purified by silica gel chromatography to give the title compound (0.62 g, yield 51%).

¹H NMR (300 MHz, CDCl₃) δ ppm: 1.71-1.90 (m, 4 H), 2.46-2.58 (m, 4 H), 3.70 (s, 2 H), 7.47 (t, J=7.8 Hz, 1 H), 7.64-7.71 (m, 1 H), 8.06-8.12 (m, 1 H), 8.20 (t, J=1.7 Hz, 1 H).

The compounds described in the following Reference Examples 315-317 were produced in the similar manner as in Reference Example 314.

### Reference Example 315

### 1-(3-nitrobenzyl)piperidine

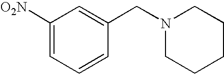

¹H NMR (300 MHz, CDCl₃) δ ppm: 1.39-1.50 (m, 2 H), 1.53-1.66 (m, 6 H), 2.34-2.43 (m, 4 H), 3.54 (s, 2 H), 7.46 (t, J=7.8 Hz, 1 H), 7.66 (d, J=7.8 Hz, 1 H), 8.05-8.12 (m, 1 H), 8.18 (t, J=1.7 Hz, 1 H).

### Reference Example 316

### 4-(3-nitrobenzyl)morpholine

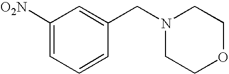

¹H NMR (300 MHz, CDCl₃) δ ppm: 2.43-2.49 (m, 4 H), 3.58 (s, 2 H), 3.69-3.75 (m, 4 H), 7.48 (t, J=7.9 Hz, 1 H), 7.67 (d, J=8.3 Hz, 1 H), 8.08-8.14 (m, 1 H), 8.21 (t, J=1.7 Hz, 1 H).

### Reference Example 317

### tert-butyl 4-(3-nitrobenzyl)-1-piperazinecarboxylate

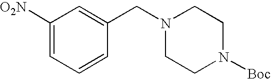

¹H NMR (300 MHz, CDCl₃) δ ppm: 1.46 (s, 9 H), 2.37-2.43 (m, 4 H), 3.41-3.47 (m, 4 H), 3.59 (s, 2 H), 7.48 (t, J=7.8 Hz, 1 H), 7.63-7.69 (m, 1 H), 8.08-8.14 (m, 1 H), 8.20 (t, J=1.7 Hz, 1 H).

### Reference Example 318

### 3-(1-pyrrolizinylmethyl)aniline

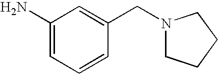

1-(3-Nitrobenzyl)pyrrolidine (0.62 g, 3.0 mmol) was dissolved in ethanol (3.0 mL), and 10% palladium-carbon (0.068 g) was added. To the reaction suspension was added dropwise hydrazine monohydrate (0.44 mL, 9.0 mmol) at room temperature at a rate that allowed reflux of the solvent due to the reaction heat, and the mixture was stirred at room temperature for 15 min. Palladium-carbon was filtered off and the reaction solution was concentrated. To the residue were added saturated aqueous solution of sodium hydrogen carbonate and ethyl acetate and the mixture was subjected to extraction. The organic layer was dried (Na₂SO₄) and concentrated to give the title compound (0.47 g, yield 89%).

¹H NMR (200 MHz, CDCl₃) δ ppm: 1.70-1.86 (m, 4 H), 2.45-2.56 (m, 4 H), 3.52 (s, 2 H), 3.62 (s, 2 H), 6.52-6.61 (m, 1 H), 6.67-6.75 (m, 2 H), 7.09 (t, J=7.9 Hz, 1 H).

The compounds described in the following Reference Examples 319-321 were produced in the similar manner as in Reference Example 318.

### Reference Example 319

### 3-(1-piperidinylmethyl)aniline

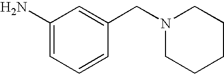

¹H NMR (300 MHz, CDCl₃) δ ppm: 1.37-1.47 (m, 2 H), 1.51-1.62 (m, 4 H), 2.30-2.43 (m, 4 H), 3.38 (s, 2 H), 3.62 (s, 2 H), 6.54-6.60 (m, 1 H), 6.67-.6.72 (m, 2 H), 7.08 (t, J=7.8 Hz, 1 H).

### Reference Example 320

### 3-(4-morpholinylmethyl)aniline

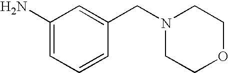

¹H NMR (300 MHz, CDCl₃) δ ppm: 2.38-2.50 (m, 4 H), 3.41 (s, 2 H), 3.64 (s, 2 H), 3.67-3.75 (m, 4 H), 6.56-6.62 (m, 1 H), 6.67-6.74 (m, 2 H), 7.09 (t, J=8.0 Hz, 1 H).

### Reference Example 321

### tert-butyl 4-(3-aminobenzyl)-1-piperazinecarboxylate

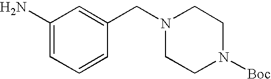

¹H NMR (300 MHz, CDCl₃) δ ppm: 1.45 (s, 9 H), 2.33-2.41 (m, 4 H), 3.38-3.45 (m, 6 H), 3.64 (s, 2 H), 6.55-6.61 (m, 1 H), 6.65-6.70 (m, 2 H), 7.08 (t, J=7.9 Hz, 1 H).

The compound described in the following Reference Example 322 was produced in the similar manner as in Reference Example 303.

### Reference Example 322

### N-[2-(4-aminophenyl)ethyl]-2,2,2-trifluoroacetamide

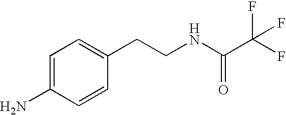

¹H NMR (300 MHz, CDCl₃) δ ppm: 2.76 (t, J=7.0 Hz, 2 H), 3.55 (q, J=6.5 Hz, 2 H), 3.63 (s, 2 H), 6.32 (s, 1 H), 6.65 (d, J=8.5 Hz, 2 H), 6.96 (d, J=8.5 Hz, 2 H).

### Reference Example 323

### 2-(2-nitrophenyl)acetamide

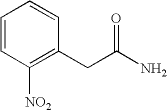

(2-Nitrophenyl)acetic acid (3.6 g, 20 mmol) was dissolved in dichloromethane (20 mL) and oxalyl chloride (3.5 mL, 40 mmol) and a catalytic amount of DMF were added at room temperature. The mixture was stirred at room temperature for 4 hrs. The reaction solution was concentrated and the residue was dissolved in THF (5.0 mL). This solution was added dropwise to 25% aqueous ammonia solution (50 mL) at room temperature. The obtained suspension was stirred at room temperature for 30 min and the precipitates were collected by filtration, washed with water and dried under reduced pressure. Ethanol was added to a crudely purified product and the mixture was stirred, filtered, washed with ethanol and dried under reduced pressure to give the title compound (2.7 g, yield 74%).

¹H NMR (200 MHz, CDCl₃) δ ppm: 3.89 (s, 2 H), 5.50 (brs, 1 H), 5.78 (brs, 1 H), 7.40-7.54 (m, 2 H), 7.56-7.68 (m, 1 H), 8.06 (d, J=8.1 Hz, 1 H).

### Reference Example 324

### 2,2,2-trifluoro-N-[2-(2-nitrophenyl)ethyl]acetamide

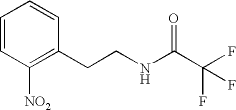

A powder of 2-(2-nitrophenyl)acetamide (2.7 g, 15 mmol) was added by small portions to 1.0 M borane-THF solution (50 mL) and the reaction solution was refluxed for 3 days. The temperature was lowered to room temperature and methanol (50 mL) was added dropwise to the reaction solution. The mixture was refluxed for 5 hrs. The reaction solution was concentrated, and the residue was dissolved in methanol (50 mL). Ethyl trifluoroacetate (2.7 mL, 23 mmol) was added to this solution at room temperature and the mixture was stirred overnight. Ethyl trifluoroacetate (2.7 mL, 23 mmol) was further added and the mixture was stirred overnight at room temperature. The solution was concentrated, and when ethyl acetate was added, insoluble materials precipitated. Thus, insoluble materials were filtered off. The mother liquor was concentrated, and the residue was purified by silica gel chromatography to give the title compound (2.2 g, yield 56%).

¹H NMR (300 MHz, CDCl₃) δ ppm: 3.17 (t, J=7.0 Hz, 2 H), 3.74 (q, J=6.7 Hz, 2 H), 6.75 (s, 1 H), 7.35-7.47 (m, 2 H), 7.59 (dt, J=7.5, 1.3 Hz, 1 H), 7.97 (dd, J=8.2, 1.3 Hz, 1 H).

### Reference Example 325

### N-[2-(2-aminophenyl)ethyl]-2,2,2-trifluoroacetamide

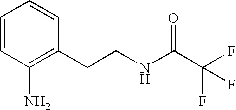

2,2,2-Trifluoro-N-[2-(2-nitrophenyl)ethyl]acetamide (2.2 g, 8.4 mmol) was dissolved in ethanol (10 mL) and 10% palladium-carbon (0.40 g) was added. The mixture was stirred under a hydrogen atmosphere for 2 days. Palladium-carbon was filtered off and the mother liquor was concentrated. The obtained solid product was dried under reduced pressure to give the title compound (1.9 g, yield 96%).

¹H NMR (300 MHz, CDCl₃) δ ppm: 2. 82 (t, J=7.0 Hz, 2 H), 3.54-3.62 (m, 2 H), 3.78 (s, 2 H), 6.73 (dd, J=7.8, 1.0 Hz, 1 H), 6.78 (dt, J=7.4, 1.2 Hz, 1 H), 7. 02 (dd, J=7.6, 1.7 Hz, 1 H), 7. 10 (dt, J=7. 6, 1.5 Hz, 1 H), 7.20 (s, 1 H).

### Reference Example 326

### N-[3-(3-aminophenyl)propyl]-2,2,2-trifluoroacetamide

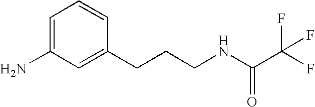

3-(3-Nitrophenyl)acrylic acid (3.9 g, 20 mmol) was dissolved in dichloromethane (20 mL) and oxalyl chloride (3.5 mL, 40 mmol) and catalytic amount of DMF were added at room temperature. The mixture was stirred at room temperature for 4 hrs. The reaction solution was concentrated and the residue was dissolved in THF (10 mL). This solution was added dropwise to 25% aqueous ammonia solution (50 mL) at room temperature. The obtained suspension was stirred at room temperature for 2 hrs. and the reaction solution was diluted with water. The precipitates were collected by filtration, washed with water and dried under reduced pressure. A crudely purified product was dissolved in ethanol (20 mL) and THF (20 mL) and 10% palladium-carbon (0.41 g) was added. The mixture was stirred overnight under a hydrogen atmosphere at 60° C. Palladium-carbon was filtered off and the mother liquor was concentrated and dried under reduced pressure. A 1.0 M borane-THF solution (60 mL) was slowly added to the obtained residue and the mixture was refluxed overnight. The reaction solution was cooled to room temperature and methanol (50 mL) was added. The mixture was further refluxed for 5 hrs. The reaction mixture was again cooled to room temperature and ethyl trifluoroacetate (2.8 mL, 23 mmol) was added. The mixture was stirred at room temperature for 2 days. The solution was concentrated and the residue was purified by silica gel chromatography to give the title compound (1.1 g, yield 23%).

¹H NMR (300 MHz, CDCl₃) δ ppm: 1.85-1.98 (m, 2 H), 2.60 (t, J=7.4 Hz, 2 H), 3.39 (q, J=6.7 Hz, 2 H), 3.64 (s, 2 H), 6.18 (s, 1 H), 6.49-6.61 (m, 3 H), 7.09 (t, J=7.7 Hz, 1 H).

### Reference Example 327

### 2,2,2-trifluoro-N-[2-(4-methoxy-3-nitrophenyl)ethyl]acetamide

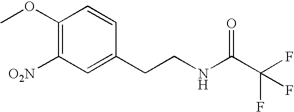

(4-Hydroxy-3-nitrophenyl)acetic acid (1.3 g, 6.6 mmol) was dissolved in dichloromethane (10 mL) and oxalyl chloride (3.5 mL, 40 mmol) and a catalytic amount of DMF were added. The mixture was stirred at room temperature for 30 min. The reaction solution was concentrated and the residue was dissolved in THF and again concentrated. 25% Aqueous ammonia solution (10 mL) and THF (5 mL) were added to the residue and the mixture was stirred at room temperature for 20 min. The obtained suspension was diluted with water and THF was evaporated under reduced pressure. The precipitates were collected by filtration, washed with water and dried under reduced pressure. A crudely purified product and potassium carbonate (1.1 g, 8.0 mmol) were suspended in acetonitrile (20 mL) and methyl iodide (0.44 mL, 7.1 mmol) was added at room temperature. The mixture was stirred for 3 hrs. Acetonitrile was evaporated under reduced pressure and DMF (5.0 mL) and methyl iodide (0.44 mL, 7.1 mmol) were added to the residue and the mixture was stirred overnight at room temperature. Water was added to the reaction solution and the obtained precipitate was filtered, washed with water and dried under a nitrogen stream. The obtained product was dissolved in dichloromethane, and the solution was dried (MgSO₄) and concentrated. To the residue was slowly added 1.0 M borane-THF solution (15 mL) at room temperature, and the mixture was refluxed overnight. The reaction solution was cooled to room temperature and methanol (15 mL) was added. The mixture was further refluxed for 5 hrs. The reaction mixture was concentrated and dissolved in methanol (5.0 mL). Ethyl trifluoroacetate (0.65 mL, 5.4 mmol) was added to the reaction solution and the mixture was stirred at room temperature for 2 hrs. Ethyl trifluoroacetate (2.0 mL, 16 mmol) was further added and the mixture was stirred overnight at room temperature. The solution was concentrated and the residue was purified by silica gel chromatography to give the title compound (0.39 g, yield 25%).

¹H NMR (300 MHz, CDCl₃) δ ppm: 2.91 (t, J=7.2 Hz, 2 H), 3.62 (q, J=7.0 Hz, 2 H), 3.96 (s, 3 H), 6.38 (s, 1 H), 7.07 (d, J=8.7 Hz, 1 H), 7.38 (dd, J=8.7, 2.3 Hz, 1 H), 7.70 (d, J=2.3 Hz, 1 H).

The compound described in the following Reference Example 328 was produced in the similar manner as in Reference Example 325.

### Reference Example 328

### N-[2-(3-amino-4-methoxyphenyl)ethyl]-2,2,2-trifluoroacetamide

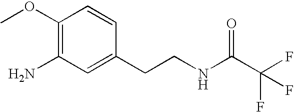

¹H NMR (300 MHz, CDCl₃) δ ppm: 2.74 (t, J=6.8 Hz, 2 H), 3.57 (q, J=6.6 Hz, 2 H), 3.82 (s, 2 H), 3.84 (s, 3 H), 6.25 (s, 1 H), 6.49-6.57 (m, 2 H), 6.73 (d, J=7.7 Hz, 1 H)

### Reference Example 329

### 4-ethoxy-3-nitrobenzaldehyde

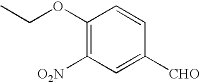

To a mixture of 4-hydroxy-3-nitrobenzaldehyde (10 g, 61 mmol) and potassium carbonate (13 g, 92 mmol) were added DMF (60 mL) and ethyl iodide (10 mL, 125 mmol) at room temperature and the mixture was stirred overnight at 70° C. and at room temperature for 2 days. Water was added to the reaction solution and the mixture was extracted with ethyl acetate. The organic layer was washed successively with 1N hydrochloric acid and saturated aqueous solution of sodium hydrogen carbonate, dried (MgSO₄), and filtered through a silica gel layer. The mother liquor was concentrated to give the title compound (12 g, yield 99%).

¹H NMR (300 MHz, CDCl₃) δ ppm: 1.53 (t, J=7.0 Hz, 3 H), 4.30 (q, J=7.1 Hz, 2 H), 7.21 (d, J=8.7 Hz, 1 H), 8.06 (dd, J=8.7, 2.1 Hz, 1 H), 8.33 (d, J=2.1 Hz, 1 H), 9.93 (s, 1 H).

### Reference Example 330

### N-(3-amino-4-ethoxybenzyl)-2,2,2-trifluoro-N-methylacetamide

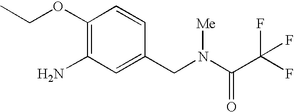

4-Ethoxy-3-nitrobenzaldehyde (4.1 g, 21 mmol) was dissolved in THF (10 mL) and 2.0 M methylamine-THF solution (11 mL) was added. The mixture was stirred overnight and the solution was concentrated. The residue was dissolved in ethanol (20 mL) and sodium borohydride (0.78 g, 20 mmol) was added at room temperature. The mixture was stirred for 2 days. To the reaction solution was added 2N hydrochloric acid (40 mL) and the mixture was stirred for 3 hrs, and 8N aqueous sodium hydroxide solution (10 mL) was further added. The mixture was extracted with dichloromethane and the organic layer was dried (MgSO₄) and concentrated. To the residue were added excess ethyl trifluoroacetate (6.0 mL, 50 mmol) and diethyl ether (2 mL) and the mixture was stirred for 3 days. The reaction solution was concentrated and purified by silica gel chromatography. The obtained crudely purified product containing impurities was suspended in diisopropyl ether and hexane, filtered, washed with hexane and dried. This crudely purified product (1.6 g, about 5.1 mmol) was dissolved in ethanol (20 mL) and 10% palladium-carbon (0.16 g) was added. To this suspension was added dropwise hydrazine monohydrate (0.74 mL, 15 mmol) at room temperature over 15 min and the mixture was stirred at room temperature for 1 hr. Palladium-carbon was filtered off and the mother liquor was concentrated. Water and ethyl acetate were added to the residue and the mixture was subjected to extraction. The organic layer was dried (MgSO₄) and concentrated. The residue was purified by silica gel chromatography to give the title compound (1.3 g).

¹H NMR (300 MHz, CDCl₃) δ ppm: 1.44 (td, J=7.0, 2. 1 Hz, 3 H) 2.88 (s, 1 H), 3.01 (d, J=1.5 Hz, 2 H), 3.85 (s, 2 H), 4.06 (qd, J=7.0, 2.3 Hz, 2 H), 4.45-4.50 (m, 2 H), 6.51-6.64 (m, 2 H), 6.69-6.7 5 (m, 1 H).

LC/MS (ESI) m/z 277 (M+H⁺).

### Reference Example 331

### N-(2,3-dihydro-1H-indol-6-ylmethyl)-2,2,2-trifluoroacetamide

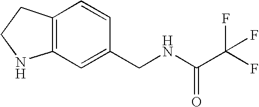

To a mixture of 1H-indole-6-carboxylic acid (1.0 g, 6.3 mmol), 1-hydroxy-1H-benzotriazoleammonium salt (1.3 g, 8.5 mmol) and WSC (1.6 g, 8.2 mmol) was added acetonitrile (12 mL) and the suspension was stirred at room temperature for 30 min. DMF (6.0 mL) was added to the reaction suspension and the mixture was stirred at room temperature for 2 days. To the reaction solution were added saturated aqueous solution of sodium hydrogen carbonate and ethyl acetate and the mixture was subjected to extraction. The organic layer was washed with 1N aqueous hydrochloric acid solution and water and dried (MgSO₄). The mother liquor was filtered by passing through a silica gel layer and concentrated under reduced pressure. To the residue was added dropwise 1.0 M borane-THF solution (25 mL) at room temperature and the mixture was refluxed overnight. The reaction solution was returned to room temperature and excess methanol (ca. 25 mL) was added at room temperature until a gas ceased to occur and the mixture was further refluxed overnight. The reaction solution was concentrated under reduced pressure, and the residue was dissolved in methanol (5.0 mL). To this solution was added ethyl trifluoroacetate (0.55 mL, 4.6 mmol) at room temperature and the mixture was stirred at room temperature for 3 days. The solution was concentrated under reduced pressure, and the residue was purified by silica gel chromatography to give the title compound (0.13 g, yield 12%).

¹H NMR (300 MHz, CDCl₃) δ ppm: 3.02 (t, J=8.4 Hz, 2 H), 3.58 (t, J=8.4 Hz, 2 H), 4.41 (d, J=5.7 Hz, 2 H), 6.47 (s, 1 H), 6.55 (s , 1 H), 6. 6 0 (dd, J=7.4, 1.4 Hz, 1 H), 7.08 (d, J=7.3 Hz, 1 H).

LC/MS (ESI) m/z 245 (M+H⁺).

### Reference Example 332

### N,N-dimethyl-1H-indole-6-carboxamide

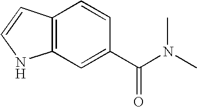

To a mixture of 1H-indole-6-carboxylic acid (2.6 g, 16 mmol), WSC (3.9 g, 20 mmol) and HOBt (3.1 g, 20 mmol) were added 2.0 M dimethylamine-THF solution (20 mL) and acetonitrile (10 mL) and the mixture was stirred at room temperature for 5 hrs. To the reaction solution were added saturated aqueous solution of sodium hydrogen carbonate and ethyl acetate and the mixture was subjected to extraction. The organic layer was filtered by passing through a silica gel layer, and concentrated under reduced pressure. A mixed solvent of ethyl acetate-diisopropyl ether-diethyl ether was added to the residue to solidify the product. The obtained solid powder was filtered, washed with diethyl ether and dried under reduced pressure to give the title compound (2.6 g, yield 86%).

¹H NMR (300 MHz, CDCl₃) δ ppm: 3.08 (s, 6 H), 6.52-6. 57 (m, 1 H), 7.15 (dd, J=8.1, 1.3 Hz, 1 H), 7.24-7.30 (m, 1 H), 7.51 (s, 1 H), 7.62 (d, J=8.3 Hz, 1 H), 8.81 (s, 1 H).

### Reference Example 333

### 1-(2,3-dihydro-1H-indol-6-yl)-N,N-dimethylmethanamine dihydrochloride

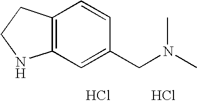

N,N-Dimethyl-1H-indole-6-carboxamide (0.72 g, 3.8 mmol) was dissolved in acetic acid (4.0 mL) and sodium cyanoborohydride (0.57 g, 9.1 mmol) was added at room temperature. The mixture was stirred for 4 hrs. Water was added to the reaction solution and the liquid was basified with 8N aqueous sodium hydroxide solution. Dichloromethane was added and the mixture was subjected to extraction. The organic layer was dried (MgSO₄) and concentrated. To the residue was added 1.0 M borane-THF solution (20 mL) at room temperature and the mixture was stirred overnight at 65° C. Methanol (20 mL) was added dropwise and the mixture was refluxed for 3 days. The reaction solution was concentrated and dissolved in ethyl acetate. This solution was filtered by passing an aminosilica gel layer and the mother liquor was concentrated. The residue was dissolved in acetic acid (4.0 mL) 5N hydrochloric acid-ethyl acetate solution (5.0 mL) was slowly added at room temperature. The mixture was stirred for a while and the obtained suspension was diluted with ethyl acetate. The precipitates were collected by filtration, washed with ethyl acetate and a mixed solvent of ethyl acetate and a small amount of ethanol under a nitrogen stream and dried with a nitrogen stream to give the title compound (0.74 g, yield 78%).

¹H NMR (300 MHz, DMSO-d₆) δ ppm: 2.67 (d, J=4.3 Hz, 6 H) 3.16 (t, J=7.6 Hz, 2 H), 3.67 (t, J=8.0 Hz, 2 H), 4.28 (d, J=3.6 Hz, 2 H), 7.33-7.47 (m, 3 H), 10.80 (s, 1 H).

LC/MS (ESI) m/z 177 (M+H⁺) −2HCl.

### Reference Example 334

### tert-butyl [4-(bromomethyl)pyridin-2-yl]carbamate

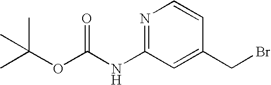

A mixture of tert-butyl [4-(hydroxymethyl)pyridin-2-yl]carbamate (1.94 g), carbon tetrabromide (4.71 g) and triphenylphosphine (2.41 g) in dichloromethane (55 mL) was stirred at room temperature for 5 hrs. The solvent was evaporated and acetonitrile was added to the residue. The precipitates were collected by filtration, washed with acetonitrile and dried to give the title compound as white crystals (1.86 g, yield 75%).

¹H NMR (300 MHz, CDCl₃) δ ppm: 1.54 (s, 9 H), 4.38 (s, 2 H), 6.99 (dd, J=5.2, 1.6 Hz, 1 H), 8.00 (s, 1 H), 8.12 (s, 1 H), 8.24 (d, J=5.3 Hz, 1 H).

### Reference Example 335

### tert-butyl {4-[(dimethylamino)methyl]pyridin-2-yl}carbamate

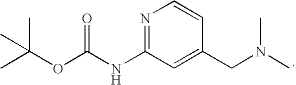

To a solution of tert-butyl [4-(bromomethyl)pyridin-2-yl]carbamate (1.86 g) in THF (5 mL) was added 50% dimethyl amine solution (15 mL) and the mixture was stirred at room temperature for 12 hrs. The reaction solution was poured into saturated aqueous solution of sodium hydrogen carbonate and the mixture was extracted with ethyl acetate. The organic layer was dried (MgSO₄) and the solvent was evaporated. The obtained residue was washed with diethyl ether to give the title compound as a white powder (0.91 g, yield 56%).

¹H NMR (300 MHz, CDCl₃) δ ppm: 1.53 (s, 9 H), 2.25 (s, 6 H) 3.42 (s, 2 H), 6.99 (dd, J=5.2, 1.4 Hz, 1 H), 7.89 (s, 1 H), 8.09 (s, 1 H), 8.21 (d, J=5.3 Hz, 1 H).

### Reference Example 336

### 4-[(dimethylamino)methyl]pyridine-2-amine dihydrochloride

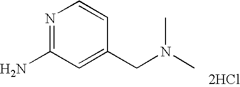

1S A mixture of tert-butyl {4-[(dimethylamino)methyl]pyridin-2-yl}carbamate (0.91 g) and 4N hydrochloric acid-ethyl acetate (20 mL) was stirred at 50° C. for 2 hrs. After cooling, the solvent was evaporated. The obtained residue was washed with ethyl acetate to give the title compound as a pale yellow powder (0.63 g, yield 78%).

¹H NMR (300 MHz, DMSO-d₆) δ ppm: 2.73 (s, 6 H), 4.33 (s, 2 H), 7.09 (s, 1 H), 7.18 (d, J=6.4 Hz, 1 H), 8.03 (d, J=6.6 Hz, 1 H), 8.36 (s, 2 H).

LC/MS (ESI) m/z 152 (M+H⁺) −2HCl.

### Reference Example 337

N-[(1R,2S)-1-({6-[(dimethylamino)methyl]-2,3-dihydro-4H-1,4-benzoxazin-4-yl)carbonyl)-2-(1H-indol-3-yl)propyl]-4-phenyl-1-piperidinecarboxamide

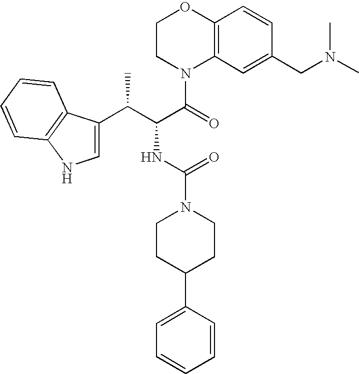

(2R,3S)-3-(1H-Indol-3-yl)-2-{[(4-phenyl-1-piperidinyl)carbonyl]amino]butanoic acid (0.11 g, 0.27 mmol) and (3,4-dihydro-2H-1,4-benzoxazin-6-ylmethyl)dimethylamine dihydrochloride (0.070 g, 0.26 mmol) and O-(7-azabenzotriazol-1-yl)-1,1,3,3-tetramethyluronium hexafluorophosphate (HATU) (0.15 g, 0.39 mmol) were dissolved in DMF (1.0 mL) and N,N-diisopropylethylamine (0.18 mL, 1.0 mmol) was added. The mixture was stirred overnight at room temperature. To the reaction solution were added saturated aqueous solution of sodium hydrogen carbonate solution and ethyl acetate and the mixture was subjected to extraction. The organic layer was filtered through an amino silica gel layer. The mother liquor was concentrated and purified by HPLC (acetonitrile/water=10/90-100/0, containing 0.1% trifluoroacetic acid). A fraction containing the objective substance was concentrated and neutralized with saturated aqueous solution of sodium hydrogen carbonate to give the title compound.

LC/MS (ESI) m/z 580 (M+H⁺).

The compounds described in the following Reference Examples 338-357 were produced in the similar manner as in Reference Example 337.

### Reference Example 338

### N-[(1R,2S)-1-({6-[(dimethylamino)methyl]-2,3-dihydro-4H-1,4-benzoxazin-4-yl}carbonyl)-2-(1H-indol-3-yl)propyl]-4-(4-fluorophenyl)-1-piperazinecarboxamide

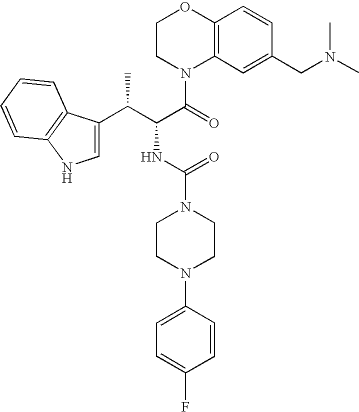

LC/MS (ESI) m/z 599 (M+H⁺).

### Reference Example 339

### N-[(1R,2S)-1-({6-[(dimethylamino)methyl]-2,3-dihydro-4H-1,4-benzoxazin-4-yl}carbonyl)-2-(1H-indol-3-yl)propyl]-4-phenylpiperazine-1-carboxamide

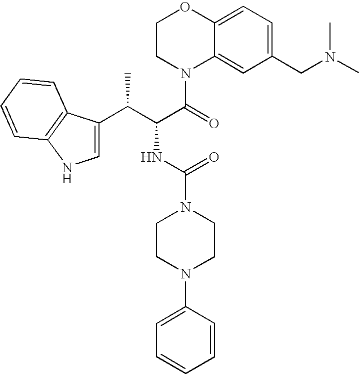

LC/MS (ESI) m/z 581 (M+H⁺).

### Reference Example 340

### N-[(1R,2S)-1-({6-[(dimethylamino)methyl]-2,3-dihydro-4H-1,4-benzoxazin-4-yl}carbonyl)-2-(1H-indol-3-yl)propyl]-4-(4-fluorophenyl)piperazine-1-carboxamide

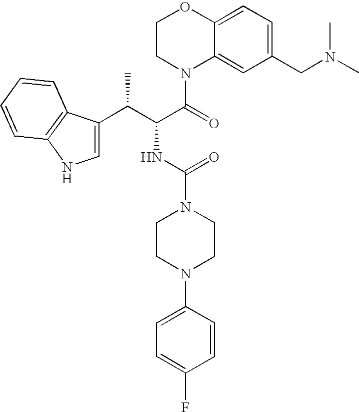

LC/MS (ESI) m/z 599 (M+H⁺).

### Reference Example 341

### N-[(1R,2S)-1-({6-[(dimethylamino)methyl]-2,3-dihydro-4H-1,4-benzoxazin-4-yl}carbonyl)-2-(1H-indol-3-yl)propyl]-4-(2-methylphenyl)piperidine-1-carboxamide

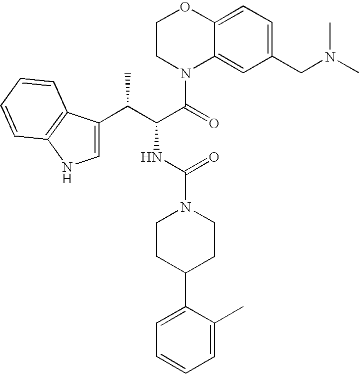

LC/MS (ESI) m/z 594 (M+H⁺)

### Reference Example 342

### N-[(1R,2S)-1-({6-[(dimethylamino)methyl]-2,3-dihydro-4H-1,4-benzoxazin-4-yl}carbonyl)-2-(1H-indol-3-yl)propyl]-4-(4-fluorophenoxy)piperidine-1-carboxamide

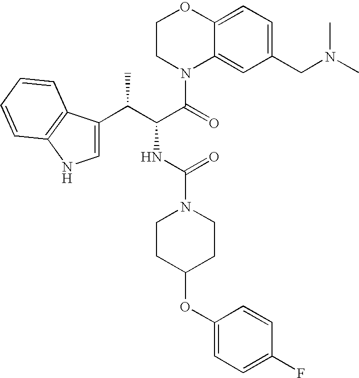

LC/MS (ESI) m/z 614 (M+H⁺).

### Reference Example 343

### 4-benzyl-N-[(1R,2S)-1-({6-[(dimethylamino)methyl]-2,3-dihydro-4H-1,4-benzoxazin-4-yl}carbonyl)-2-(1H-indol-3-yl)propyl]piperidine-1-carboxamide

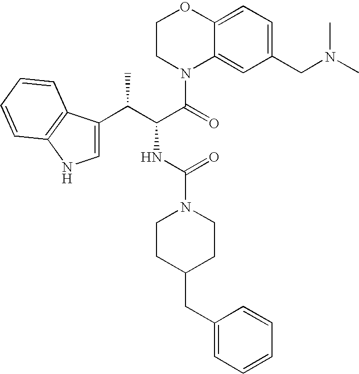

LC/MS (ESI) m/z 594 (M+H⁺).

### Reference Example 344

### 4-benzyl-N-[(1R,2S)-1-({6-[(dimethylamino)methyl]-2,3-dihydro-4H-1,4-benzoxazin-4-yl}carbonyl)-2-(1H-indol-3-yl)propyl]piperazine-1-carboxamide

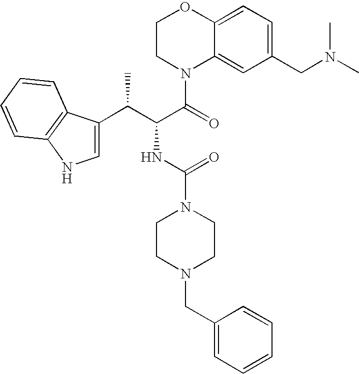

LC/MS (ESI) m/z 595 (M+H⁺).

### Reference Example 345

### 4-benzoyl-N-[(1R,2S)-1-((6-[(dimethylamino)methyl]-2,3-dihydro-4H-1,4-benzoxazin-4-yl}carbonyl)-2-(1H-indol-3-yl)propyl]piperazine-1-carboxamide

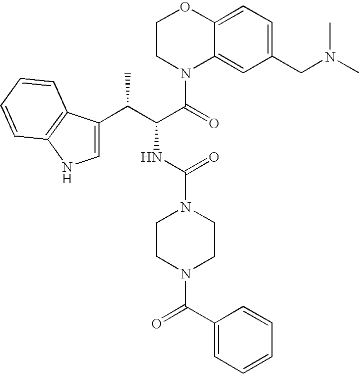

LC/MS (ESI) m/z 608 (M+H⁺).

### Reference Example 346

### N-[(1R,2S)-1-({6-[(dimethylamino)methyl]-2,3-dihydro-4H-1,4-benzothiazin-4-yl)carbonyl)-2-(1H-indol-3-yl)propyl]-4-phenylpiperidine-1-carboxamide

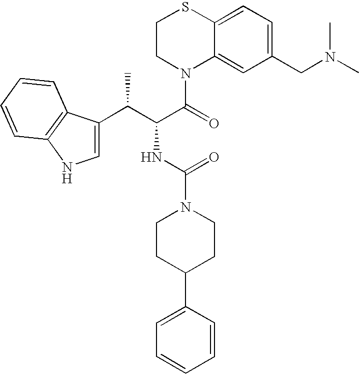

LC/MS (ESI) m/z 596 (M+H⁺).

### Reference Example 347

### N-[(1R,2S)-1-({6-[(dimethylamino)methyl]-2,3-dihydro-4H-1,4-benzothiazin-4-yl}carbonyl)-2-(1H-indol-3-yl)propyl]-4-phenylpiperazine-1-carboxamide

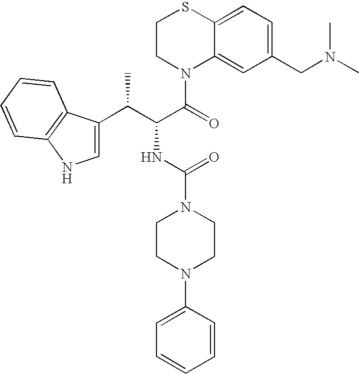

LC/MS (ESI) m/z 597 (M+H⁺).

### Reference Example 348

### N-[(1R,2S)-1-((6-[(dimethylamino)methyl]-2,3-dihydro-4H-1,4-benzothiazin-4-yl}carbonyl)-2-(1H-indol-3-yl)propyl]-4-(4-fluorophenyl)piperidine-1-carboxamide

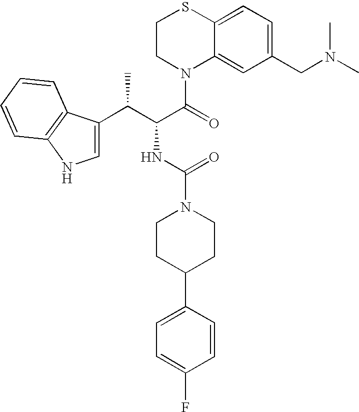

LC/MS (ESI) m/z 614 (M+H⁺).

### Reference Example 349

### N-[(1R,2S)-1-({6-[(dimethylamino)methyl]-2,3-dihydro-4H-1,4-benzothiazin-4-yl)carbonyl)-2-(1H-indol-3-yl)propyl]-4-(4-fluorophenyl)piperazine-1-carboxamide

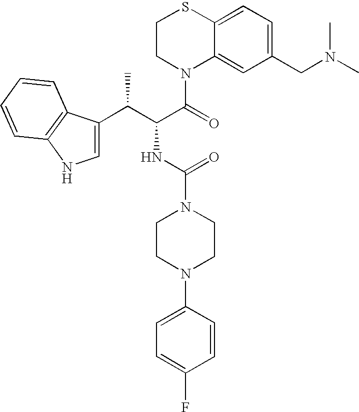

LC/MS (ESI) m/z 615 (M+H⁺).

### Reference Example 350

### N-[(1R,2S)-1-({6-[(dimethylamino)methyl]-2,3-dihydro-4H-1,4-benzothiazin-4-yl}carbonyl)-2-(1H-indol-3-yl)propyl]-4-(2-methylphenyl)piperidine-1-carboxamide

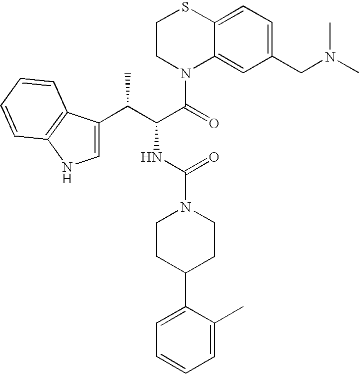

LC/MS (ESI) m/z 610 (M+H⁺).

### Reference Example 351

### N-[(1R,2S)-1-({6-[(dimethylamino)methyl]-2,3-dihydro-4H-1,4-benzothiazin-4-yl}carbonyl)-2-(1H-indol-3-yl)propyl]-4-(2-methylphenyl)piperazine-1-carboxamide

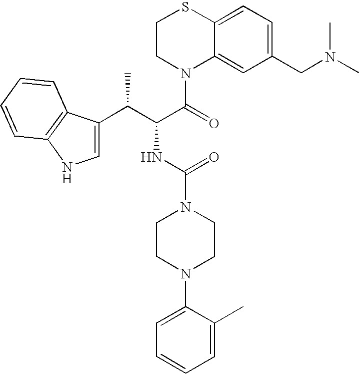

LC/MS (ESI) m/z 611 (M+H⁺).

### Reference Example 352

### N-[(1R,2S)-1-({6-[(dimethylamino)methyl]-2,3-dihydro-4H-1,4-benzothiazin-4-yl}carbonyl)-2-(1H-indol-3-yl)propyl]-4-(4-fluoro-2-methylphenyl)piperazine-1-carboxamide

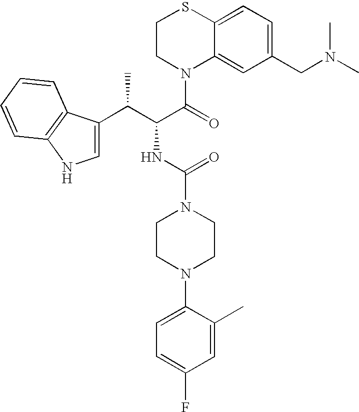

LC/MS (ESI) m/z 629 (M+H⁺).

### Reference Example 353

### N-[(1R,2S)-1-{[7-[(dimethylamino)methyl]-3,4-dihydroquinolin-1(2H)-yl]carbonyl}-2-(1H-indol-3-yl)propyl]-4-phenylpiperidine-1-carboxamide

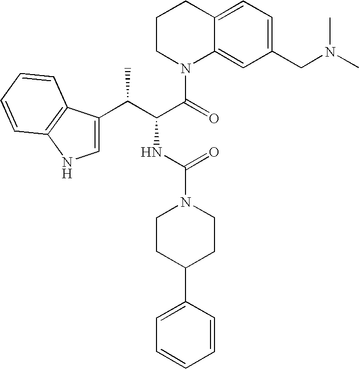

LC/MS (ESI) m/z 578 (M+H⁺).

### Reference Example 354

### N-[(1R,2S)-1-{[7-[(dimethylamino)methyl]-3,4-dihydroquinolin-1(2H)-yl]carbonyl}-2-(1H-indol-3-yl)propyl]-4-(4-fluorophenyl)piperidine-1-carboxamide

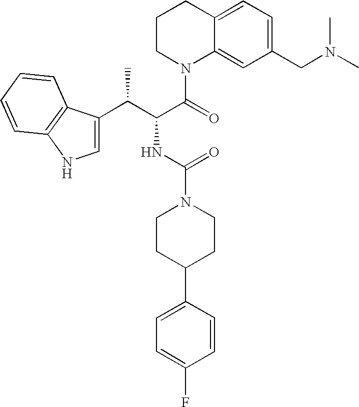

LC/MS (ESI) m/z 596 (M+H⁺).

### Reference Example 355

### N-[(1R,2S)-1-{[7-[(dimethylamino)methyl]-3,4-dihydroquinolin-1(2H)-yl]carbonyl}-2-(1H-indol-3-yl)propyl]-4-(4-fluorophenyl)piperazine-1-carboxamide

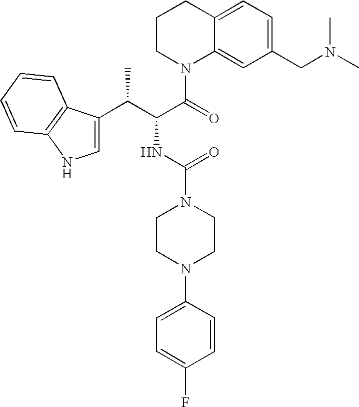

LC/MS (ESI) m/z 597 (M+H⁺).

### Reference Example 356

### N-[(1R,2S)-17{[7-[(dimethylamino)methyl]-3,4-dihydroquinolin-1(2H)-yl]carbonyl}-2-(1H-indol-3-yl)propyl]-4-(2-methylphenyl)piperazine-1-carboxamide

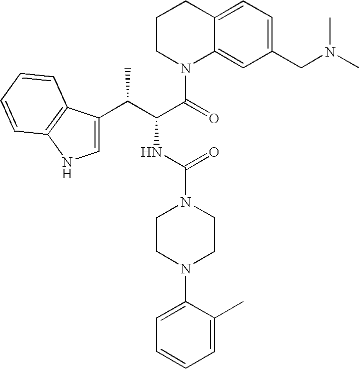

LC/MS (ESI) m/z 593 (M+H⁺).

### Reference Example 357

### N-[(1R,2S)-1-{[7-[(dimethylamino)methyl]-3,4-dihydroquinolin-1(2H)-yl]carbonyl}-2-(1H-indol-3-yl)propyl]-4-(4-fluorophenoxy)piperidine-1-carboxamide

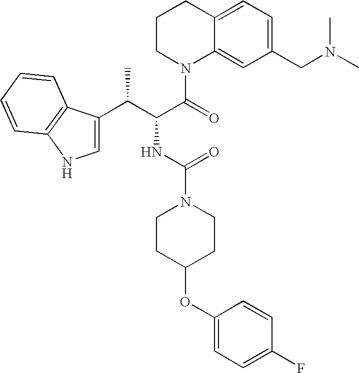

LC/MS (ESI) m/z 612 (M+H⁺).

### Reference Example 358

### N-[(1R,2S)-1-{[6-(aminomethyl)-2,3-dihydro-1H-indol-1-yl]carbonyl}-2-(1H-indol-3-yl)propyl]-4-phenyl-1-piperidinecarboxamide

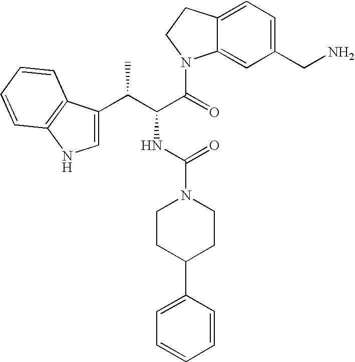

(2R,3S)-3-(1H-Indol-3-yl)-2-{[(4-phenyl-1-piperidinyl)carbonyl]amino}butanoic acid (0.082 g, 0.20 mmol) and N-(2,3-dihydro-1H-indol-6-ylmethyl)-2,2,2-trifluoroacetamide (0.049 g, 0.20 mmol) and O-(7-azabenzotriazol-1-yl)-1,1,3,3-tetramethyluronium hexafluorophosphate (HATU) (0.13 g, 0.33 mmol) were dissolved in DMF (1.0 mL) and N,N-diisopropylethylamine (0.06 mL, 0.35 mmol) was added. The mixture was stirred at room temperature for 3 days. To the reaction solution were added 1N aqueous hydrochloric acid solution and ethyl acetate and the mixture was subjected to extraction. The organic layer was filtered through a silica gel layer. The mother liquor was concentrated, and the residue was dissolved in methanol (6.0 mL) and 10% aqueous potassium carbonate solution (2.0 mL) was added at room temperature. The mixture was stirred overnight. Methanol was evaporated from the reaction solution under reduced pressure and water was added to the obtained suspension. The precipitates were collected by filtration, washed with water and concentrated under reduced pressure to give the title compound (0.12 g, 99%), which was purified by HPLC (acetonitrile/water=10/90-100/0, containing 0.1% trifluoroacetic acid). A fraction containing the objective substance was concentrated and neutralized with saturated aqueous solution of sodium hydrogen carbonate to give the title compound.

LC/MS (ESI) m/z 536 (M+H⁺).

The compound described in the following Reference Example 359 was produced in the similar manner as in Reference Example 358.

### Reference Example 359

### N-[(1R,2S)-1-{[6-(aminomethyl)-2,3-dihydro-1H-indol-1-yl]carbonyl]-2-(1H-indol-3-yl)propyl]-4-(4-fluoro-2-methylphenyl)-1-piperazinecarboxamide

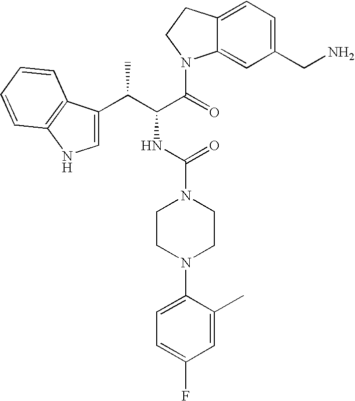

LC/MS (ESI) m/z 569 (M+H⁺)

The compounds described in the following Reference Examples 360-361 were produced in the similar manner as in Example 285.

### Reference Example 360

### N-[(1R,2S)-1-({6-[(dimethylamino)methyl]-2,3-dihydro-1H-indol-1-yl}carbonyl)-2-(1H-indol-3-yl)propyl]-4-(4-fluoro-2-methylphenyl)-1-piperazinecarboxamide

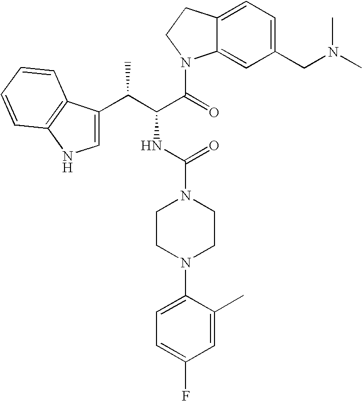

LC/MS (ESI) m/z 597 (M+H⁺).

### Reference Example 361

### N-[(1R,2S)-1-((6-[(dimethylamino)methyl]-2,3-dihydro-1H-indol-1-yl}carbonyl)-2-(1H-indol-3-yl)propyl]-4-phenyl-1-piperidinecarboxamide

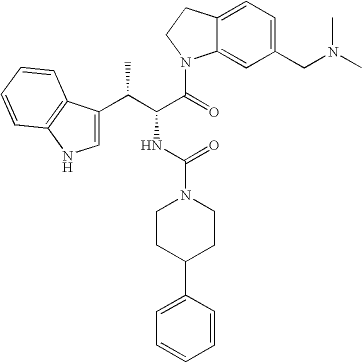

LC/MS (ESI) m/z 597 (M+H⁺).

The compounds described in the following Reference Examples 362-366 were produced in the similar manner as in Reference Example 337.

### Reference Example 362

### N-[(1R,2S)-1-(16-[(dimethylamino)methyl]-2,3-dihydro-1H-indol-1-yl}carbonyl)-2-(1H-indol-3-yl)propyl]-4-(4-fluorophenyl)-1-piperazinecarboxamide

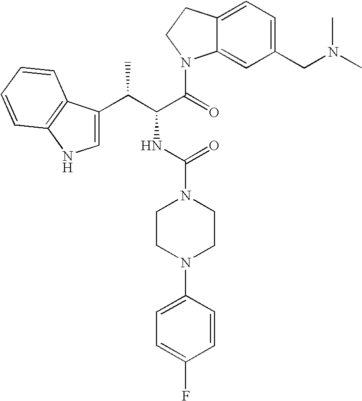

LC/MS (ESI) m/z 583 (M+H⁺).

### Reference Example 363

### N-[(1R,2S)-1-({6-[(dimethylamino)methyl]-2,3-dihydro-1H-indol-1-yl}carbonyl)-2-(1H-indol-3-yl)propyl]-4-(2-methylphenyl)-1-piperazinecarboxamide

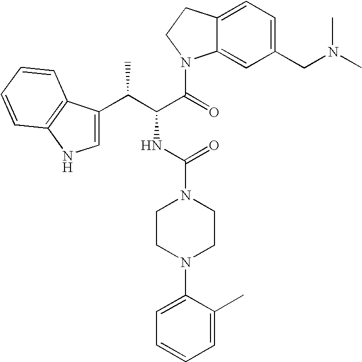

LC/MS (ESI) m/z 579 (M+H⁺).

### Reference Example 364

### N-[(1R,2S)-1-({6-[(dimethylamino)methyl]-2,3-dihydro-1H-indol-1-yl}carbonyl)-2-(1H-indol-3-yl)propyl3-4-(4-fluorophenyl)-3-oxo-1-piperazinecarboxamide

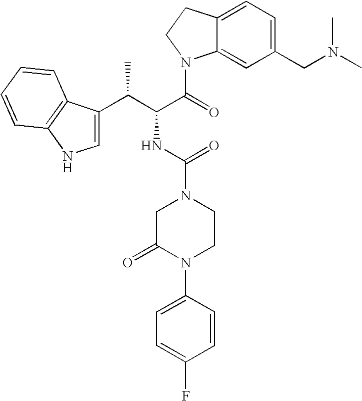

LC/MS (ESI) m/z 597 (M+H⁺).

### Reference Example 365

### N-[ (1R,2S)-1-({6-[(dimethylamino)methyl]-2,3-dihydro-1H-indol-1-yl}carbonyl)-2-(1H-indol-3-yl)propyl]-4-(4-fluoro-2-methylphenyl)-3-oxo-1-piperazinecarboxamide

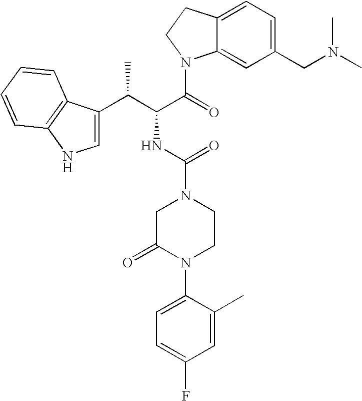

LC/MS (ESI) m/z 611 (M+H⁺).

### Reference Example 366

### N-[(1R,2S)-1-((6-[(dimethylamino)methyl]-2,3-dihydro-1H-indol-1-yl]carbonyl)-2-(1H-indol-3-yl)propyl]-4-(4-fluoro-2-formylphenyl)-1-piperazinecarboxamide

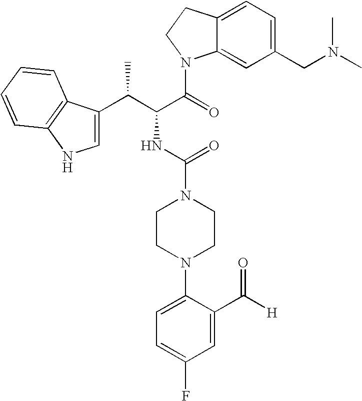

LC/MS (ESI) m/z 611 (M+H⁺).

### Reference Example 367

### N-[(1R,2S)-1-((6-[(dimethylamino)methyl]-2,3-dihydro-1H-indol-1-yl}carbonyl)-2-(1H-indol-3-yl)propyl]-4-[4-fluoro-2-(hydroxymethyl)phenyl]-1-piperazinecarboxamide

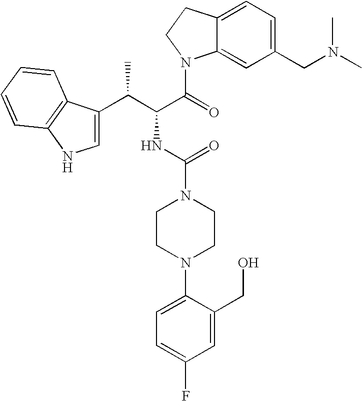

N-[(1R,2S)-1-({6-[(Dimethylamino)methyl]-2,3-dihydro-1H-indol-1-yl~carbonyl)-2-(1H-indol-3-yl)propyl]-4-(4-fluoro-2-formylphenyl)-1-piperazinecarboxamide (0.10 g, 0.17 mmol) was dissolved in ethanol (1.0 mL) and sodium borohydride (0.011 g, 0.29 mmol) was added at room temperature. The mixture was stirred overnight. To the reaction solution were added saturated aqueous solution of sodium hydrogen carbonate and water, and the obtained suspension was filtered. The product was washed with water and dried to give the title compound (0.083 g, yield 80%).

LC/MS (ESI) m/z 613 (M+H⁺).

In the following Reference Examples 368-370, ¹H-NMR spectrum is a δ value in ppm as measured using tetramethylsilane as an internal standard and Brooker DPX 300 (300 MHz) type spectrometer or JNM-AL400 type nuclear magnetic resonance equipment (manufactured by JEOL Ltd.). In addition, enantiomeric excess (% ee) and diastereomeric excess (% de) were measured by high performance liquid chromatography under the following conditions.
[high performance liquid chromatography conditions]
column: CHIRALCEL OJ-R (150 mmL×4.6 mm ID) (manufactured by DAICEL CHEMICAL INDUSTRIES, LTD.)
mobile phase: 0-05M KH₂PO₄ (pH 6.5)/MeCN (75:25)
flow rate: 0.5 ml/min
detection: UV (254 nm)
temperature: 15° C.

### Reference Example 368

### Ethyl (2R,3S)-2-amino-3-(1H-indol-3-yl)butanoate O,O′-diacetyl-L-tartarate

Ethyl (2RS,3SR)-2-amino-3-(1H-indol-3-yl)butanoate (1400 g), isopropyl alcohol (25 L), water (2.8 L) and O,O′-diacetyl-L-tartaric acid (1331 g) were dissolved at room temperature. A seed crystal was added and the mixture was stirred at the same temperature for 2 hrs. The mixture was ice-cooled and stirred for 4 hrs., and left standing at the same temperature for 20 hrs. The crystallized crystals were separated, washed with isopropyl alcohol (6 L), and vacuum dried to give the title compound as white crystals (1005 g). yield 37%. As a result of high performance liquid chromatography analysis, the diastereomeric excess was 74% de.

¹H-NMR(300 MHz,DMSO-d₆): δ 11.01 (s, 1H), 7.51 (d, J=7.8 Hz, 1H), 7.37 (d, J=8.0 Hz, 1H), 7.19 (d, J=2.4 Hz, 1H), 7.09 (d, J=7.5 Hz, 1H), 7.00 (d, J=7.4 Hz, 1H), 5.32 (s, 2H), 4.03 (d, J=6.5 Hz, 1H), 3.96 (q, J=7.1 Hz, 2H), 3.58-3.49 (m, 1H), 2.04 (s, 6H), 1.40 (d, J=7.1 Hz, 3H), 0.96 (t, J=7.1 Hz, 3H).

### Reference Example 369

### Ethyl (2R,3S)-2-amino-3-(1H-indol-3-yl)butanoate O,O′-diacetyl-L-tartarate

Ethyl (2R,3S)-2-amino-3-(1H-indol-3-yl)butanoate O,O—-diacetyl-L-tartarate (50.0 g, 78% de), isopropyl alcohol (150 mL) and water (50 mL) were suspended and stirred at 50° C. for 30 min. and allowed to cool at room temperature while stirring for 1 hr. Isopropyl alcohol (300 mL) was added dropwise and the mixture was stirred at the same temperature for 2 hrs. The crystals were collected by filtration, washed with isopropyl alcohol (100 mL) and vacuum dried to give the title compound as white crystals (38.9 g). yield 78%. As a result of high performance liquid chromatography analysis, the diastereomeric excess was 95% de.

### Reference Example 370

### ethyl (2R, 3S)-2-amino-3-(1H-indol-3-yl)butanoate methanesulfonate

A mixture of toluene (50 mL) and 2N aqueous sodium hydroxide solution (50 ml) was cooled to 0-5° C. Ethyl (2R,3S)-2-amino-3-(1H-indol-3-yl)butanoate O,O′-diacetyl-L-tartarate (10 g, 79.8% de) was added and the mixture was stirred at 0-10° C. for 1 hr. After standing, the reaction mixture was partitioned and the organic layer was washed with water (50 mL×2). The organic layer was concentrated under reduced pressure, ethyl acetate (50 mL) was added and again concentrated under reduced pressure. The residue was dissolved in a mixture of ethyl acetate (135 mL) and ethanol (15 mL) and methanesulfonic acid (2.1 g) was added dropwise at 20-30° C. A seed crystal was added and the mixture was stirred at 20-30° C. for 2 hrs. The crystallized crystals were collected by filtration, washed with ethyl acetate (20 mL), and vacuum dried at 50° C. to give the title compound as white crystals (4.79 g). yield 67%. As a result of high performance liquid chromatography analysis, the enantiomeric excess was 99.0% ee.

### Example 1

### N-((1R,2S)-1-(((3-((dimethylamino)methyl)phenyl)amino)carbonyl)-2-(1H-indol-3-yl)propyl)-4-phenyl-1-piperidinecarboxamide

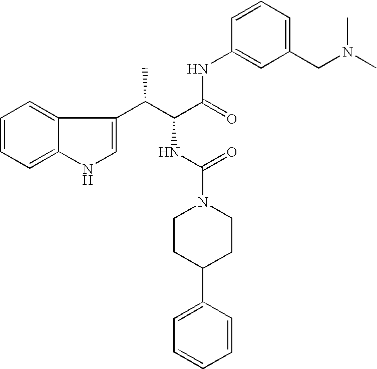

To a mixed solution of (2R,3S)-3-(1H-indol-3-yl)-2-(((4-phenyl-1-piperidinyl)carbonyl)amino)butanoic acid (95 mg), 3-((dimethylamino)methyl)aniline dihydrochloride (50 mg), WSC (65 mg) and HOBt (45 mg) in acetonitrile (1 mL)-THF (1 mL) was added triethylamine (0.062 mL), and the mixture was stirred at room temperature for 16 hours. The reaction solution was added to an aqueous solution of 10% sodium carbonate (1.5 mL) and extracted with ethyl acetate (3 mL). The extract was dried (MgSO₄) and the solvent was removed by evaporation under reduced pressure. The residue was purified by column chromatography (aminopropyl silica gel, developing solvent: hexane/ethyl acetate=5/1 to 1/1 to 1/4 to ethyl acetate). The obtained residue was washed with ethyl acetate/IPE, and the title compound was obtained as white crystals (48 mg, yield 40%).

¹H NMR (300 MHz, CDCl₃) δ ppm: 1.57 (d, J=7.2 Hz, 3 H), 1.62-1.68 (m, 2 H), 1.83 (d, J=12.0 Hz, 2 H), 2.20 (s, 6 H), 2.60-2.70 (m, 1 H), 2.78-2.94 (m, 2 H), 3.30 (s, 2 H), 3.54-3.64 (m, 1 H), 4.03 (d, J=13.7 Hz, 1 H), 4.15 (d, J=12.7 Hz, 1 H), 4.82 (t, J=8.4 Hz, 1 H), 5.48 (d, J=8.1 Hz, 1 H), 6.93 (d, J=7.3 Hz, 1 H),.6.99-7.03 (m, 2 H), 7.08-7.23 (m, 6 H), 7.27-7.34 (m, 4 H), 7.72 (s, 1 H), 7.76 (d, J=1.8 Hz, 1 H), 8.39 (m, 1 H).

LC/MS (ESI) m/z 538 (M+H⁺)

The following compounds mentioned in Examples 2 to 88 were synthesized according to the same method as Example 1.

### Example 2

### N-( (1R,2S)-1-( ((3-((dimethylamino)methyl)phenyl)amino)carbonyl)-2-(1H-indol-3-yl)propyl)-4-(4-fluorophenoxy)-1-piperidinecarboxamide

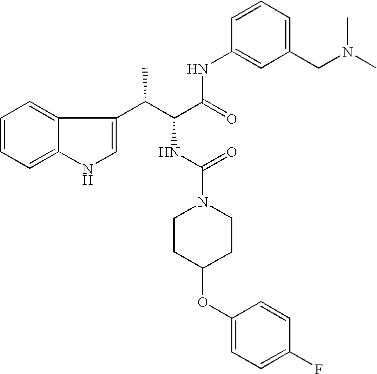

¹H NMR (300 MHz, DMSO-d₆) δ ppm: 1.30 (d, J=7.1 Hz, 3 H), 1.35-1.55 (m, 2 H), 1.78-1.93 (m, 2 H), 2.13 (s, 6 H), 3.04-3.22 (m, 2 H), 3.33 (s, 2 H), 3.53-3.64 (m, 1 H), 3.64-3.80 (s, 2 H), 4.40-4.53 (m, 1 H), 4.61 (t, J=8.7 Hz, 1 H) 6.54 (d, J=8.8 Hz, 1 H), 6.84-7.03 (m, 4 H), 7.04-7.17 (m, 3 H), 7.22-7.29 (m, 2 H), 7.31-7.40 (m, 2 H), 7.58 (d, J=7.8 Hz, 1 H), 9.81 (s, 1 H), 10.75-10.82 (m, 1 H).

LC/MS (ESI) m/z 572 (M+H⁺).

### Example 3

### 4-benzyl-N-((1R,2S)-1-(((3-((dimethylamino)methyl)phenyl)amino)carbonyl)-2-(1H-indol-3-yl)propyl)-1-piperazinecarboxamide

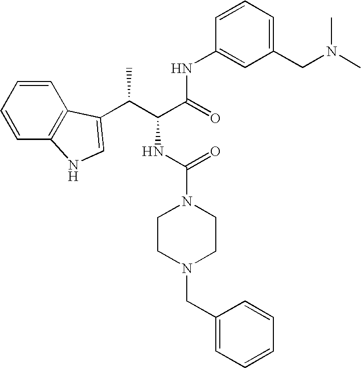

LC/MS (ESI) m/z 553 (M+H⁺).

### Example 4

### 4-benzyl-N-((1R,2S)-1-(((3-((dimethylamino)methyl)phenyl)amino)carbonyl)-2-(1H-indol-3-yl)propyl)-1-piperidinecarboxamide

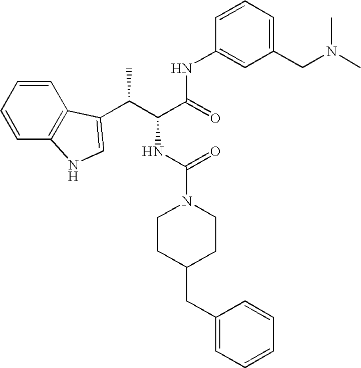

LC/MS (ESI) m/z 552 (M+H⁺).

### Example 5

### 0N-((1R,2S)-1-(((3-((dimethylamino)methyl)phenyl)amino)carbonyl)-2-(1H-indol-3-yl)propyl)-4-phenyl-1-piperazinecarboxamide

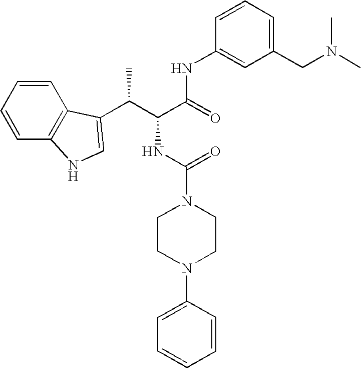

LC/MS (ESI) m/z 539 (M+H⁺).

### Example 6

### N-((1R,2S)-1-(((4-chloro-3-((dimethylamino)methyl)phenyl)amino)carbonyl)-2-(1H-indol-3-yl)propyl)-4-(4-fluorophenoxy)-1-piperidinecarboxamide

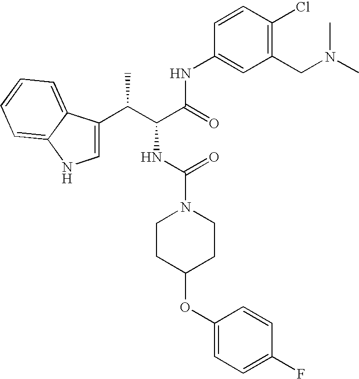

LC/MS (ESI) m/z 606 (M+H⁺).

### Example 7

### N-((1R,2S)-1-(((2-chloro-5-((dimethylamino)methyl)phenyl)amino)carbonyl)-2-(1H-indol-3-yl)propyl)-4-(4-fluorophenoxy)-1-piperidinecarboxamide

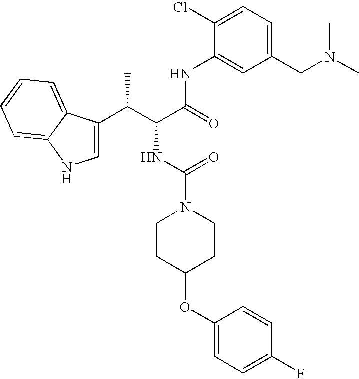

LC/MS (ESI) m/z 606 (M+H⁺).

### Example 8

### N-((1R,2S)-1-(((3-((dimethylamino)methyl)-4-methylphenyl)amino)carbonyl)-2-(1H-indol-3-yl)propyl)-4-(4-fluorophenoxy)-1-piperidinecarboxamide

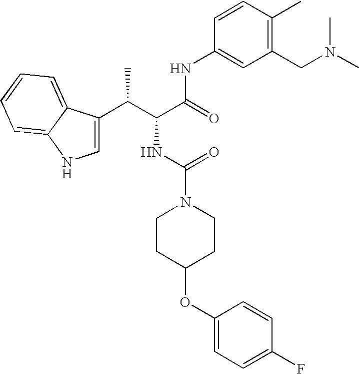

LC/MS (ESI) m/z 586 (M+H⁺).

### Example 9

### N-((1R,2S)-1-(((5-((dimethylamino)methyl)-2-methylphenyl)amino)carbonyl)-2-(1H-indol-3-yl)propyl)-4-(4-fluorophenoxy)-1-piperidinecarboxamide

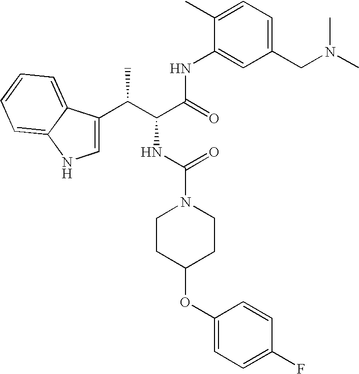

LC/MS (ESI) m/z 586 (M+H⁺).

### Example 10

### N-((1R,2S)-1-(((3-((dimethylamino)methyl)-4-fluorophenyl)amino)carbonyl)-2-(1H-indol-3-yl)propyl)-4-(4-fluorophenoxy)-1-piperidinecarboxamide

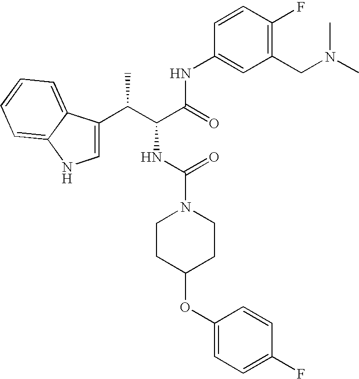

LC/MS (ESI) m/z 590 (M+H⁺).

### Example 11

### N-((1R,2S)-1-(((5-((dimethylamino)methyl)-2-fluorophenyl)amino)carbonyl)-2-(1H-indol-3-yl)propyl)-4-(4-fluorophenoxy)-1-piperidinecarboxamide

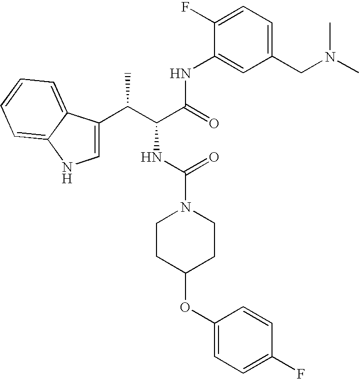

LC/MS (ESI) m/z 590 (M+H⁺).

### Example 12

### N-((IR,2S)-1-(((3-((dimethylamino)methyl)phenyl)amino)carbonyl)-2-(1H-indol-3-yl)propyl)-4-(4-fluorophenyl)-1-piperidinecarboxamide

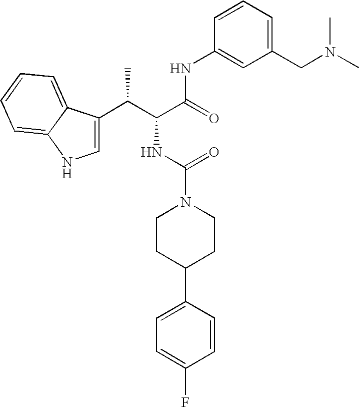

LC/MS (ESI) m/z 556 (M+H⁺).

### Example 13

### 4-(4-chlorophenyl)-N-((1R,2S)-1-(((3-((dimethylamino)methyl)phenyl)amino)carbonyl)-2-(1H-indol-3-yl)propyl)-1-piperidinecarboxamide

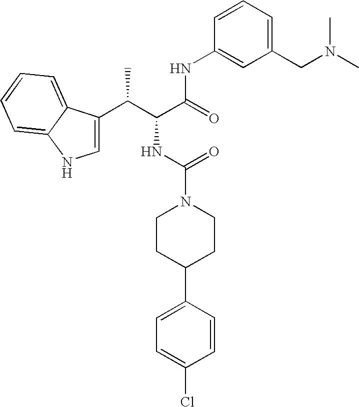

LC/MS (ESI) m/z 572 (M+H⁺).

### Example 14

### N-( (1R,2S) -1-(((3-((dimethylamino)methyl)phenyl)amino)carbonyl)-2-(1H-indol-3-yl)propyl)-4-(4-methylphenyl)-1-piperidinecarboxamide

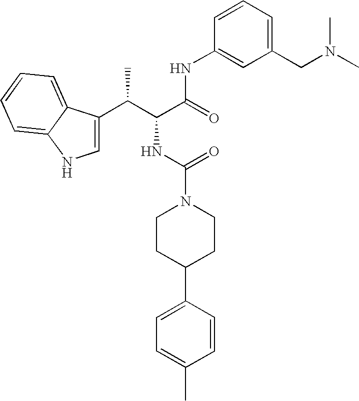

LC/MS (ESI) m/z 552 (M+H⁺)

### Example 15

### N-( (1R,2S)-1-(((3-((dimethylamino)methyl)phenyl)amino)carbonyl)-2-(1H-indol-3-yl)propyl)-4-(4-methoxyphenyl)-1-piperidinecarboxamide

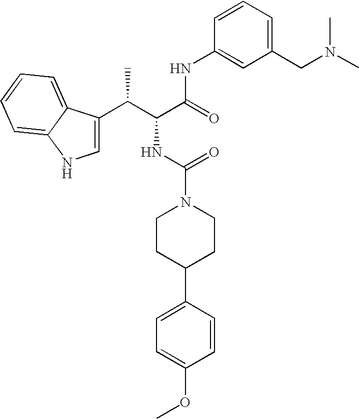

LC/MS (ESI) m/z 568 (M+H⁺).

### Example 16

### N-( (1R) -2-((3-((dimethylamino)methyl)phenyl)amino)-1-(1H-indol-3-ylmethyl)-2-oxoethyl)-4-phenyl-1-piperidinecarboxamide

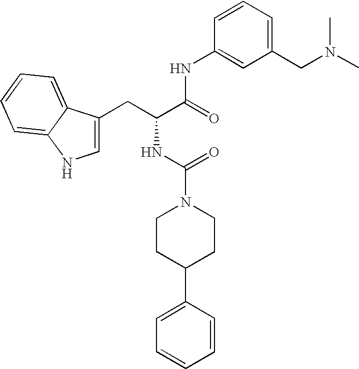

LC/MS (ESI) m/z 524 (M+H⁺).

### Example 17

### N-((1R)-2-((3-((dimethylamino)methyl)phenyl)amino)-1-(1H-indol-3-ylmethyl)-2-oxoethyl)-4-(4-fluorophenoxy)-1-piperidinecarboxamide

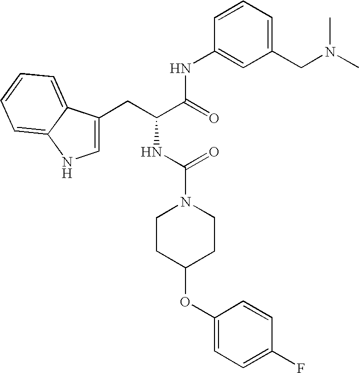

LC/MS (ESI) m/z 558 (M+H⁺).

### Example 18

### tert-butyl 4-((((1R,2S)-1-(((3-((dimethylamino)methyl)phenyl)amino)carbonyl)-2-(1H-indol-3-yl)propyl)amino)carbonyl)-1-piperazinecarboxylate

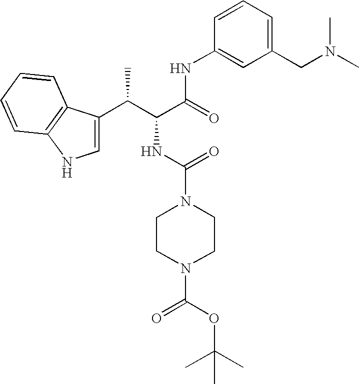

LC/MS (ESI) m/z 563 (M+H⁺).

### Example 19

### N-((1R,2S)-2-(1H-indol-3-yl)-1-(((3-(1-pyrrolidinylmethyl)phenyl)amino)carbonyl)propyl)-4-phenyl-1-piperidinecarboxamide

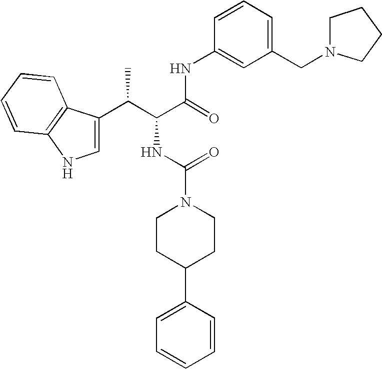

LC/MS (ESI) m/z 564 (M+H⁺).

### Example 20

### b 4-(4-fluorophenyl)-N-((1R,2S)-2-(1H-indol-3-yl)-1-(((3-(1-pyrrolidinylmethyl)phenyl)amino)carbonyl)propyl)-1-piperidinecarboxamide

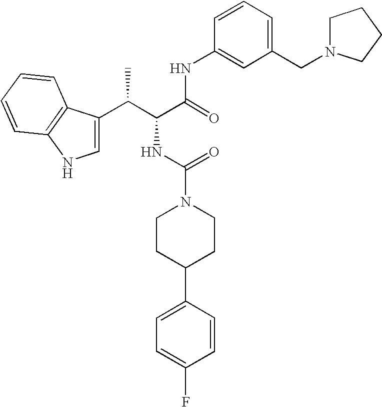

LC/MS (ESI) m/z 582 (M+H⁺).

### Example 21

### 4-(4-fluorophenoxy)-N-((1R,2S)-2-(1H-indol-3-yl)-1-(((3-(1-pyrrolidinylmethyl)phenyl)amino)carbonyl)propyl)-1-piperidinecarboxamide

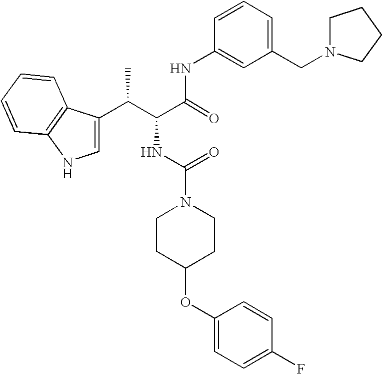

LC/MS (ESI) m/z 598 (M+H⁺).

### Example 22

### N-((1R,2S)-1-(((3-((dimethylamino)methyl)phenyl)amino)carbonyl)-2-(1H-indol-3-yl)propyl)-4-(3-fluorophenyl)-1-piperidinecarboxamide

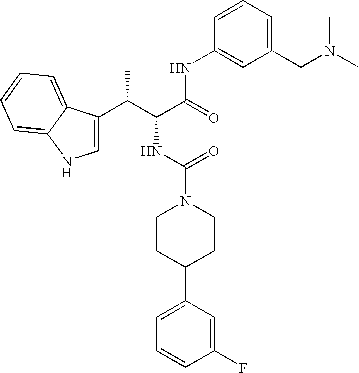

LC/MS (ESI) m/z 556 (M+H⁺).

### Example 23

### 4-(2,4-difluorophenyl)-N-((1R,2S)-1-(((3-((dimethylamino)methyl)phenyl)amino)carbonyl)-2-(1H-indol-3-yl)propyl)-1-piperidinecarboxamide

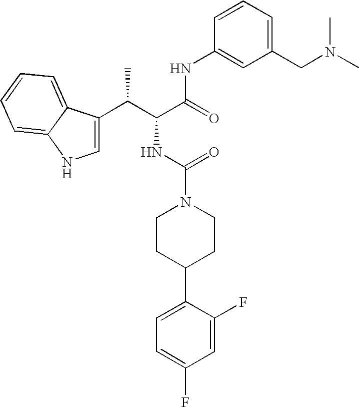

LC/MS (ESI) m/z 574 (M+H⁺).

### Example 24

### N-((1R,2S)-1-(((3-((dimethylamino)methyl)phenyl)amino)carbonyl)-2-(1H-indol-3-yl)propyl)-4-((4-fluorophenyl)thio)-1-piperidinecarboxamide

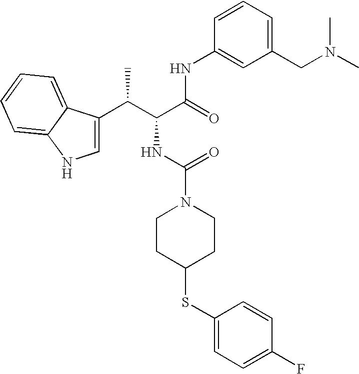

LC/MS (ESI) m/z 588 (M+H⁺).

### Example 25

### N-((1R,2S)-1-(((3-((dimethylamino)methyl)phenyl)amino)carbonyl)-2-(1H-indol-3-yl)propyl)-4-((4-fluorophenyl)sulfonyl)-1-piperidinecarboxamide

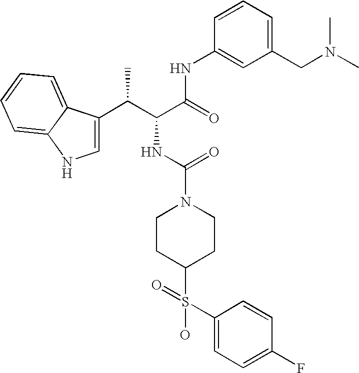

LC/MS (ESI) m/z 620 (M+H⁺).

### Example 26

### N-( (1R,2S)-1-( ((3-((dimethylamino)methyl)phenyl)amino)carbonyl)-2-(1H-indol-3-yl)propyl)-4-(4-fluorobenzoyl)-1-piperidinecarboxamide

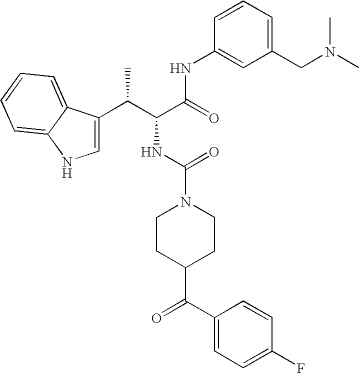

LC/MS (ESI) m/z 584 (M+H⁺).

### Example 27

### N-((1R,2S)-1-(((3-((dimethylamino)methyl)phenyl)amino)carbonyl)-2-(1H-indol-3-yl)propyl)-4-((4-fluorophenyl)sulfonyl)-1-piperazinecarboxamide

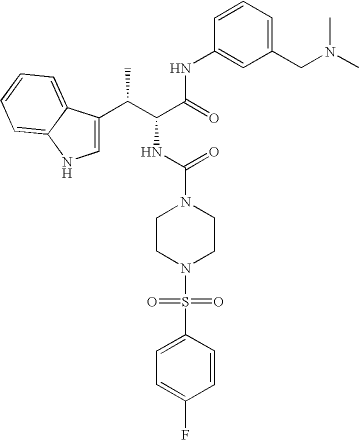

LC/MS (ESI) m/z 621 (M+H⁺).

### Example 28

### N-((1R,2S)-2-(1H-indol-3-yl)-1-(((3-(1-piperidinylmethyl)phenyl)amino)carbonyl)propyl)-4-phenyl-1-piperidinecarboxamide

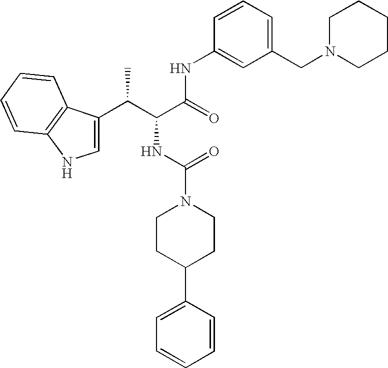

LC/MS (ESI) m/z 578 (M+H⁺).

### Example 29

### N-((1R,2S)-2-(1H-indol-3-yl)-1-(((3-(4-morpholinylmethyl)phenyl)amino)carbonyl)propyl)-4-phenyl-1-piperidinecarboxamide

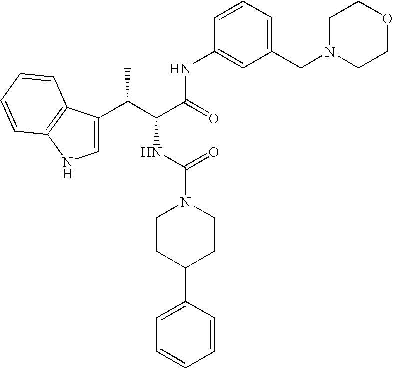

LC/MS (ESI) m/z 580 (M+H⁺).

### Example 30

### N-((1R,2S)-1-(((3-((dimethylamino)methyl)phenyl)amino)carbonyl)-2-(1H-indol-3-yl)propyl)-3,4-dihydro-2(1H)-isoquinolinecarboxamide

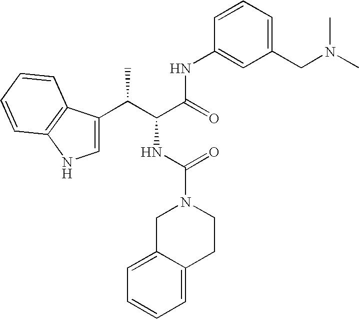

LC/MS (ESI) m/z 510 (M+H⁺).

### Example 31

### N-((1R,2S)-1-(((3-((dimethylamino)methyl)phenyl)amino)carbonyl)-2-(1H-indol-3-yl)propyl)-6-methyl-3,4-dihydro-2(1H)-isoquinolinecarboxamide

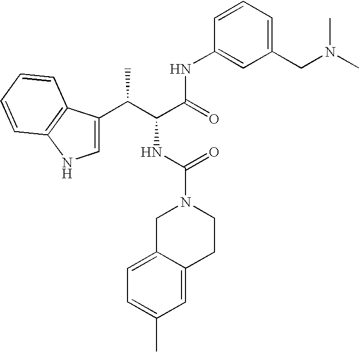

LC/MS (ESI) m/z 524 (M+H⁺).

### Example 32

### 6-chloro-N-((1R,2S)-1-(((3-((dimethylamino)methyl)phenyl)amino)carbonyl)-2-(1H-indol-3-yl)propyl)-3,4-dihydro-2(1H)-isoquinolinecarboxamide

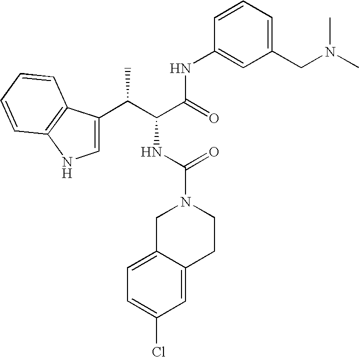

LC/MS (ESI) m/z 544 (M+H⁺).

### Example 33

### N-((1R,2S)-1-(((3-((dimethylamino)methyl)phenyl)amino)carbonyl)-2-(1H-indol-3-yl)propyl)-4,7-dihydrothieno[2,3-c]pyridine-6(5H)-carboxamide

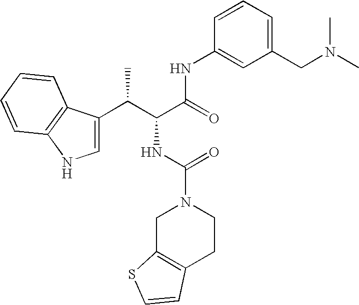

LC/MS (ESI) m/z 516 (M+H⁺).

### Example 34

### N-((1R,2S)-1-(((3-((dimethylamino)methyl)phenyl)amino)carbonyl)-2-(1H-indol-3-yl)propyl)-6-fluoro-3,4-dihydro-2(1H)-isoquinolinecarboxamide

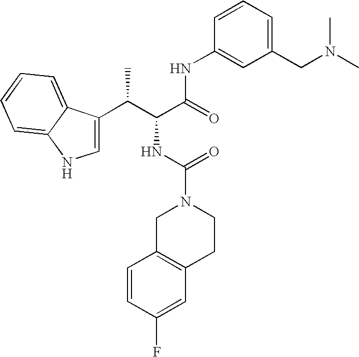

LC/MS (ESI) m/z 528 (M+H⁺).

### Example 35

### N-((1R,2S)-1-(((3-((dimethylamino)methyl)-5-(trifluoromethyl)phenyl)amino)carbonyl)-2-(1H-indol-3-yl)propyl)-4-phenyl-1-piperidinecarboxamide

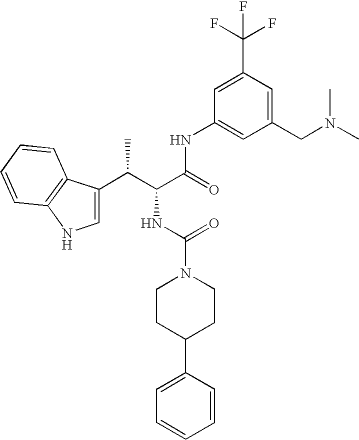

LC/MS (ESI) m/z 606 (M+H⁺).

### Example 36

### N-((1R,2S)-1-(((2-chloro-5-((dimethylamino).methyl)phenyl)amino)carbonyl)-2-(1H-indol-3-yl)propyl)-4-phenyl-1-piperidinecarboxamide

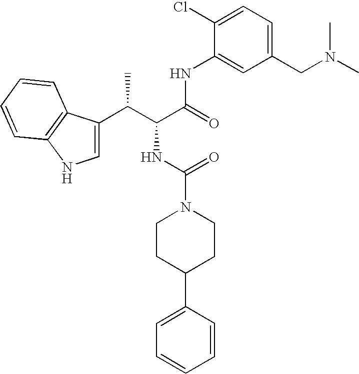

LC/MS (ESI) m/z 572 (M+H⁺).

### Example 37

### N-((1R,2S)-1-(((5-((dimethylamino)methyl)-2-methoxyphenyl)amino)carbonyl)-2-(1H-indol-3-yl)propyl)-4-phenyl-1-piperidinecarboxamide

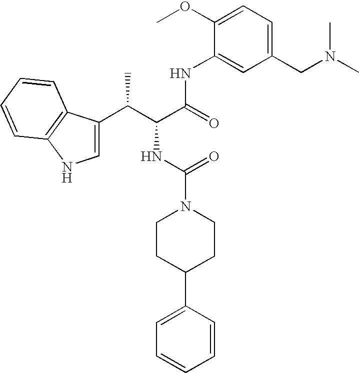

¹H NMR (300 MHz, CDCl₃) δ ppm: 1.57 (d, J=7.2 Hz, 3 H), 1.60-1.70 (m, 2 H), 1.82-1.87 (m, 2 H), 2.20 (s, 6 H), 2.61-2.72 (m, 1 H) , 2.81-2.95 (m, 2 H), 3.31 (s, 2 H), 3.50-3.61 (m, 4 H), 4.04-4.17 (m, 2 H), 4.94 (t, J=8.0 Hz, 1 H) 5.38-5.41 (m, 1 H), 6.65 (d, J=8.5 Hz, 1 H), 6.91 (dd, J=8.4, 2.1 Hz, 1 H), 7.08 (d, J=2.4 Hz, 1 H), 7.10-7.24 (m, 5 H) 7.29-7.34 (m, 3 H), 7.78-7.81 (m, 2 H), 8.10-8.13 (m, 2 H)

LC/MS (ESI) m/z 568 (M+H⁺).

### Example 38

### N-((1R,2S)-1-(((5-((dimethylamino)methyl)-2-fluorophenyl)amino)carbonyl)-2-(1H-indol-3-yl)propyl)-4-phenyl-1-piperidinecarboxamide

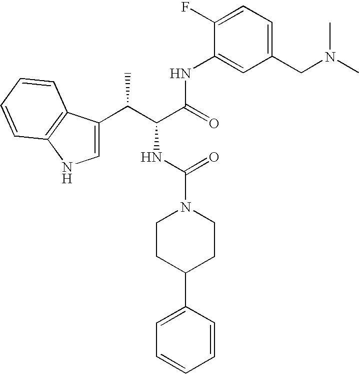

LC/MS (ESI) m/z 556 (M+H⁺).

### Example 39

### N-((1R,2S)-1-(((5-((dimethylamino)methyl)-2-methylphenyl)amino)carbonyl)-2-(1H-indol-3-yl)propyl)-4-phenyl-1-piperidinecarboxamide

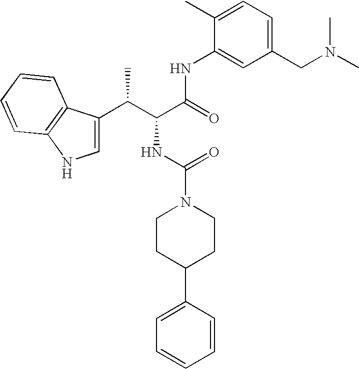

LC/MS (ESI) m/z 552 (M+H⁺).

### Example 40

### N-((1R,2S)-1-(((3-((dimethylamino)methyl)phenyl)amino)carbonyl)-2-(1H-indol-3-yl)propyl)-4-(2-methylphenyl)-1-piperidinecarboxamide

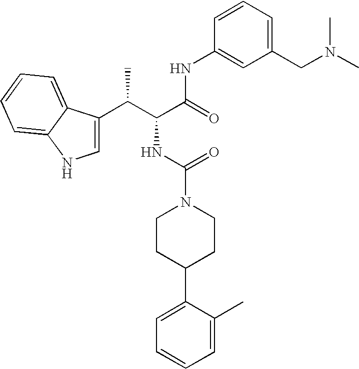

¹H NMR (400 MHz, DMSO-d₆) δ ppm: 1.33 (d, J=6.8 Hz, 3 H), 1.37-1.51 (m, 2 H), 1.60 (m, 2 H), 2.14 (s, 6 H), 2.31 (s, 3 H), 2.74-2.92 (m, 3 H), 3.30 (s, 2 H), 3.60-3.65 (m, 1 H), 4.16 (dd, J=22.8, 14.0 Hz, 2H), 4.66 (t, J=8.4 Hz, 1 H), 6.45 (d, J=8.5 Hz, 1 H), 6.89-7.18 (m, 8 H), 7.26-7.29 (m, 2 H), 7.37-7.39 (m, 1 H), 7.42 (s, 1 H), 7.62 (d, J=7.8 Hz, 1 H), 9.81 (s, 1 H), 10.80 (d, J=2.0 Hz, 1 H).

LC/MS (ESI) m/z 552 (M+H⁺).

### Example 41

### 4-(4-chlorophenyl)-N-((1R,2S)-1-(((3-((dimethylamino)methyl)phenyl)amino)carbonyl)-2-(1H-indol-3-yl)propyl)-3,6-dihydro-1(2H)-pyridinecarboxamide

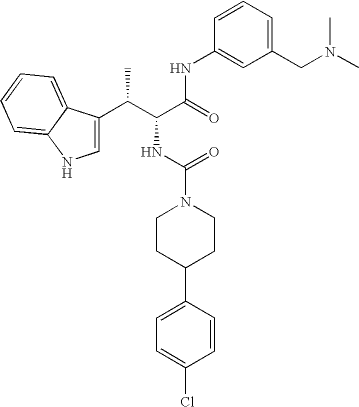

LC/MS (ESI) m/z 570 (M+H⁺).

### Example 42

### N-((1R,2S)-1-(((3-((dimethylamino)methyl)phenyl)amino)carbonyl)-2-(1H-indol-3-yl)propyl)-4-(3-(trifluoromethyl)phenyl)-1-piperidinecarboxamide

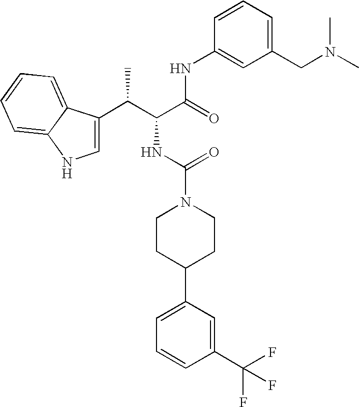

LC/MS (ESI) m/z 606 (M+H⁺).

### Example 43

### 4-(4-chlorophenyl)-N-((1R,2S)-1-(((3-((dimethylamino)methyl)phenyl)amino)carbonyl)-2-(1H-indol-3-yl)propyl)-4-hydroxy-1-piperidinecarboxamide

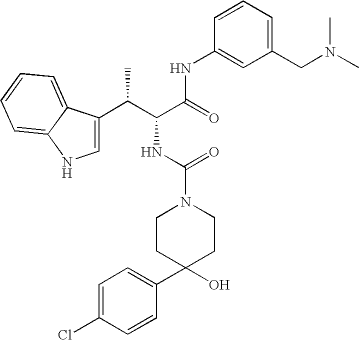

¹H NMR (400 MHz, CDCl₃) δ ppm: 1.58 (d, J=7.1 Hz, 3 H), 1.65-2.04 (m, 5 H), 2.32 (s, 6 H), 3.19-3.34 (m, 2 H), 3.46 (s, 2 H), 3.54-3.63 (m, 1 H), 3.78-3.93 (m, 2 H), 4.81 (t, J=8.4 Hz, 1 H), 5.54 (brd, J=7.6 Hz, 1 H), 6.96 (d, J=6.9 Hz, 1 H), 7.03 (s, 1 H), 7.07-7.18 (m, 5 H), 7.30-7.37 (m, 5 H), 7.73 (brs, 1 H), 7.77 (d, J=7.8 Hz, 1 H), 8.65 (brs, 1 H).

LC/MS (ESI) m/z 588 (M+H⁺).

### Example 44

### N-((1R,2S)-1-(((3-((dimethylamino)methyl)phenyl)amino)carbonyl)-2-(1H-indol-3-yl)propyl)-4-hydroxy-4-phenyl-1-piperidinecarboxamide

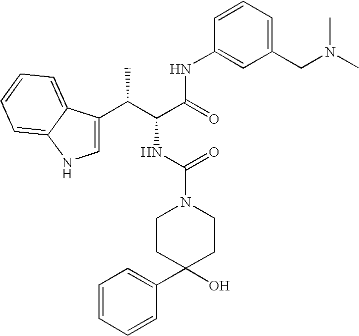

¹H NMR (400 MHz, CDCl₃) δ ppm: 1.57 (d, J=7.1 Hz, 3 H), 1.64-1.74 (m, 3 H), 1.91 (td, J=12.9, 4.6 Hz, 1 H), 2.02 (td, J=12.9, 4.6 Hz, 1 H), 2.19 (s, 6 H), 3.26 (td, J=12.9, 2.2 Hz, 1 H), 3.30 (s, 2 H), 3.34 (td, J=12.9, 2.2 Hz, 1 H), 3.56-3.64 (m, 1 H), 3.82 (brd, J=12.9 Hz, 1 H), 3.93 (brd, J=12.9 Hz, 1 H), 4.83 (t, J=8.3 Hz, 1 H), 5.52 (brd, J=7.6 Hz, 1 H), 6.94 (d, J=7.6 Hz, 1 H), 7.01 (s, 1 H), 7.09-7.45 (m, 11 H), 7.68 (brs, 1 H), 7.79 (d, J=7.8 Hz, 1 H), 8.39 (brs, 1 H).

LC/MS (ESI) m/z 554 (M+H⁺).

### Example 45

### N-((1R,2S)-1-(((3-((dimethylamino)methyl)phenyl)amino)carbonyl)-2-(1H-indol-3-yl)propyl)-4-(3-methylphenyl)-1-piperidinecarboxamide

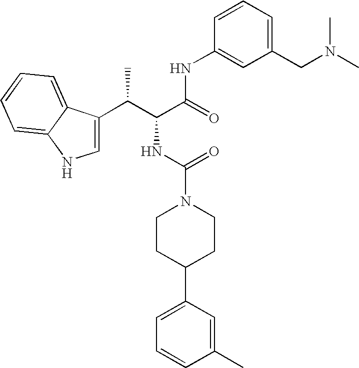

LC/MS (ESI) m/z 552 (M+H⁺).

### Example 46

### N-((1R,2S)-1-(((3-((dimethylamino)methyl)phenyl)amino)carbonyl)-2-(1H-indol-3-yl)propyl)-4-(3-methoxyphenyl)-1-piperidinecarboxamide

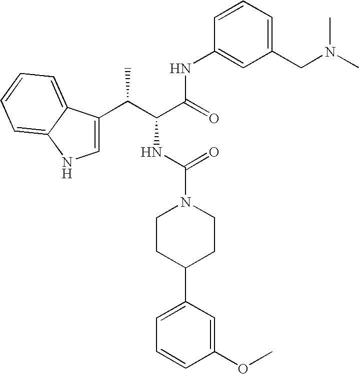

LC/MS (ESI) m/z 568 (M+H⁺).

### Example 47

### N-((1R,2S)-1-(((3-((dimethylamino)methyl)phenyl)amino)carbonyl)-2-(1H-indol-3-yl)propyl)-1-indolinecarboxamide

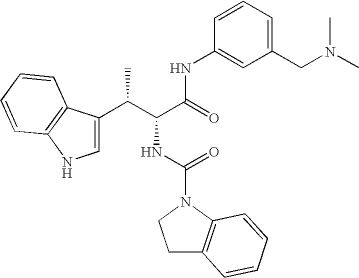

LC/MS (ESI) m/z 496 (M+H⁺).

### Example 48

### N-((1R,2S)-1-(((3-((dimethylamino)methyl)phenyl)amino)carbonyl)-2-(1H-indol-3-yl)propyl)-1,3-dihydro-2H-isoindole-2-carboxamide

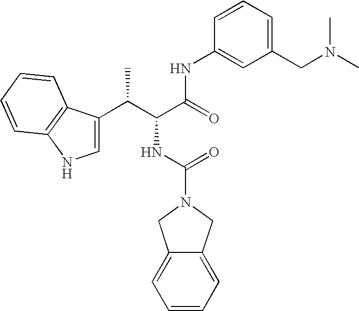

LC/MS (ESI) m/z 496 (M+H⁺).

### Example 49

### N-((1R,2S)-1-(((3-((dimethylamino)methyl)phenyl)amino)carbonyl)-2-(1H-indol-3-yl)propyl)-4-(1-naphthyl)-1-piperidinecarboxamide

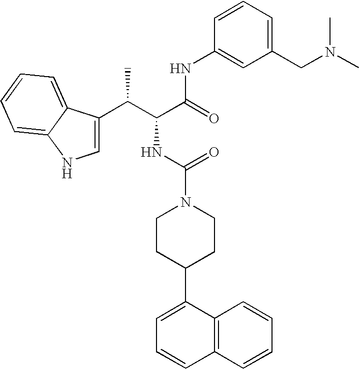

LC/MS (ESI) m/z 588 (M+H⁺).

### Example 50

### N-((1R,2S)-1-(((3-((dimethylamino)methyl)phenyl)amino)carbonyl)-2-(1H-indol-3-yl)propyl)-4-(4-fluorophenyl)-3,6-dihydro-1(2H)-pyridinecarboxamide

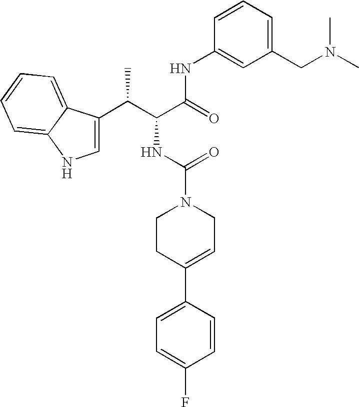

LC/MS (ESI) m/z 554 (M+H⁺).

### Example 51

### N-((1R,2S)-1-(((3-((dimethylamino)methyl)phenyl)amino)carbonyl)-2-(1H-indol-3-yl)propyl)-4-(2-fluorophenyl)-1-piperidinecarboxamide

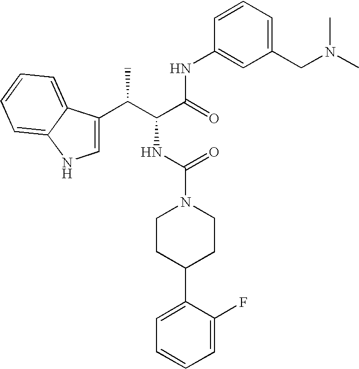

LC/MS (ESI) m/z 556 (M+H⁺).

### Example 52

### N-((1R,2S)-1-(((3-((dimethylamino)methyl)phenyl)amino)carbonyl)-2-(1H-indol-3-yl)propyl)-4-(2-(trifluoromethyl)phenyl)-3,6-dihydro-1(2H)-pyridinecarboxamide

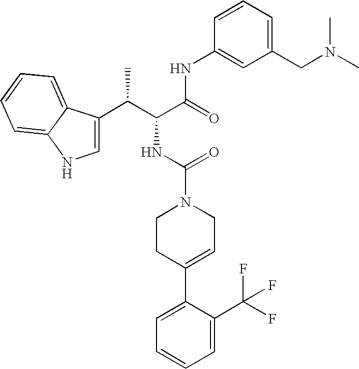

LC/MS (ESI) m/z 604 (M+H⁺).

### Example 53

### N-((1R,2S)-1-(((3-((diethylamino)methyl)phenyl)amino)carbonyl)-2-(1H-indol-3-yl)propyl)-4-phenyl-1-piperidinecarboxamide

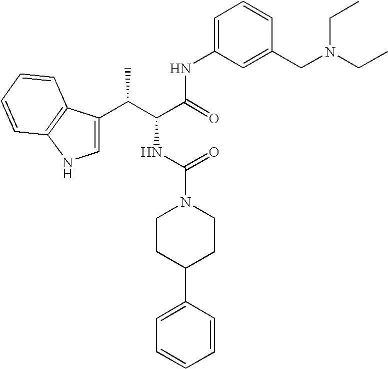

LC/MS (ESI) m/z 566 (M+H⁺).

### Example 54

### N-((1R,2S)-1-(((5-((dimethylamino)methyl)-2-ethoxyphenyl)amino)carbonyl)-2-(1H-indol-3-yl)propyl)-4-phenyl-1-piperidinecarboxamide

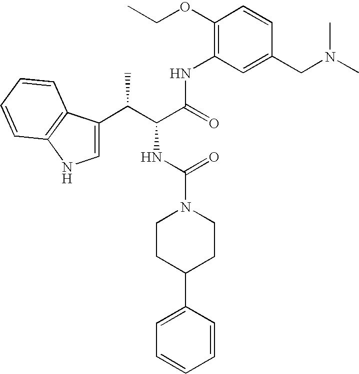

¹H NMR (300 MHz, CDCl₃) δ ppm: 1.23 (t, J=7.0 Hz, 3 H), 1.52-1.73 (m, 5 H), 1.80-1.84 (m, 2 H), 2.20 (s, 6 H), 2.64 (tt, J=12.0, 3.5 Hz, 1 H), 2.77-2.93 (m, 2 H), 3.32 (s, 2 H), 3.65 (dq, J=7.5, 7.3 Hz, 1 H), 3.81-3.93 (m, 2 H), 3.98-4.03 (m, 1 H), 4.10-4.14 (m, 1 H), 4.89 (t, J=7.5 Hz, 1 H), 5.31 (d, J=7.8 Hz, 1 H), 6.68 (d, J=8.3 Hz, 1 H), 6.91 (dd, J=2.2, 8.3 Hz, 1 H), 7.05 -7.17 (m, 5 H), 7.20-7.33 (m, 4 H), 7.75 (d, J=7.6 Hz, 1 H), 7.96 (s, 1 H), 8.05 (s, 1 H), 8.18 (d, J=2.0 Hz, 1 H).

LC/MS (ESI) m/z 582 (M+H⁺).

### Example 55

### N-((1R,2S)-1-(((5-((dimethylamino)methyl)-2-isopropoxyphenyl)amino)carbonyl)-2-(1H-indol-3-yl)propyl)-4-phenyl-1-piperidinecarboxamide

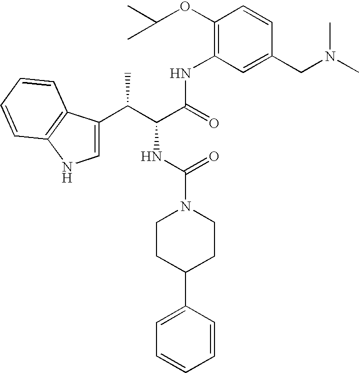

LC/MS (ESI) m/z 596 (M+H⁺).

### Example 56

### N-((1R,2S)-1-(((5-((dimethylamino)methyl)-2-ethylphenyl)amino)carbonyl)-2-(1H-indol-3-yl)propyl)-4-phenyl-1-piperidinecarboxamide

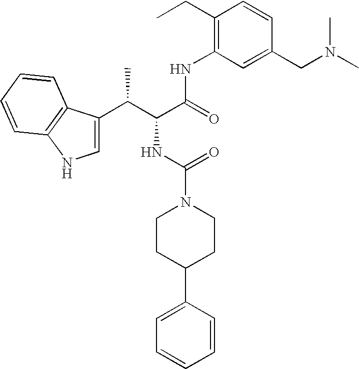

LC/MS (ESI) m/z 566 (M+H⁺).

### Example 57

### N-((1R,2S)-1-(((3-((dimethylamino)methyl)-2-methylphenyl)amino)carbonyl)-2-(1H-indol-3-yl)propyl)-4-phenyl-1-piperidinecarboxamide

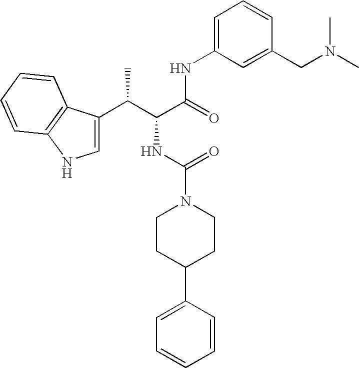

LC/MS (ESI) m/z 552 (M+H⁺).

### Example 58

### N-((1R,2S)-1-(((3-((dimethylamino)methyl)phenyl)amino)carbonyl)-2-(1H-indol-3-yl)propyl)-1,2,4,5-tetrahydro-3H-3-benzazepine-3-carboxamide

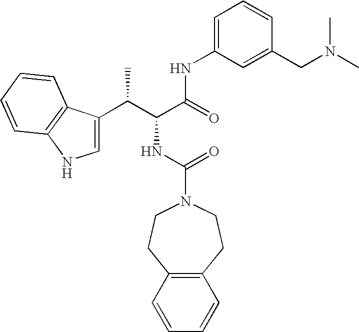

LC/MS (ESI) m/z 524 (M+H⁺).

### Example 59

### N-((1R,2S)-1-(((5-((dimethylamino)methyl)-2-propoxyphenyl)amino)carbonyl)-2-(1H-indol-3-yl)propyl)-4-phenyl-1-piperidinecarboxamide

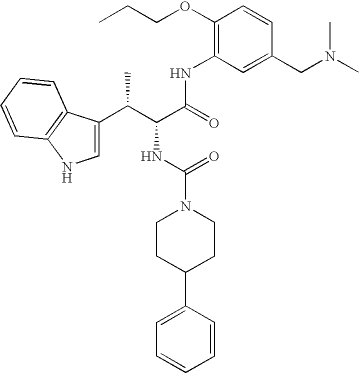

LC/MS (ESI) m/z 596 (M+H⁺).

### Example 60

### N-((1R,2S)-1-(((5-((dimethylamino)methyl)-2-(trifluoromethoxy)phenyl)amino)carbonyl)-2-(1H-indol-3-yl)propyl)-4-phenyl-1-piperidinecarboxamide

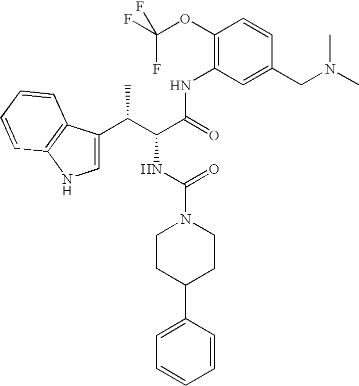

LC/MS (ESI) m/z 622 (M+H⁺).

### Example 61

### N-((1R,2S)-1-(((5-((dimethylamino)methyl)-2-(2-methoxyethoxy)phenyl)amino)carbonyl)-2-(1H-indol-3-yl)propyl)-4-phenyl-1-piperidinecarboxamide

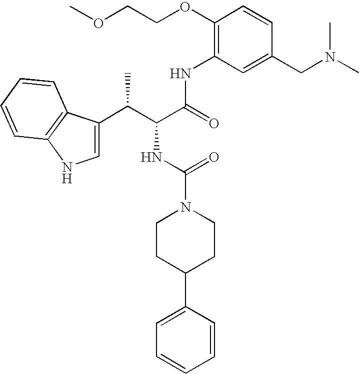

LC/MS (ESI) m/z 612 (M+H⁺).

### Example 62

### N-((1R,2S)-1-(((3-((dimethylamino)methyl)phenyl)amino)carbonyl)-2-(1H-indol-3-yl)propyl)-4-(2-methylphenyl)-1-piperazinecarboxamide

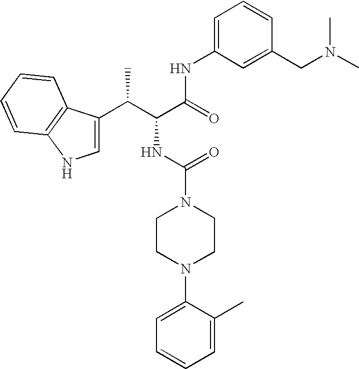

LC/MS (ESI) m/z 553 (M+H⁺).

### Example 63

### N-((1R,2S)-1-(((5-((dimethylamino)methyl)-2-methoxyphenyl)amino)carbonyl)-2-(1H-indol-3-yl)propyl)-4-(2-methylphenyl)-1-piperazinecarboxamide

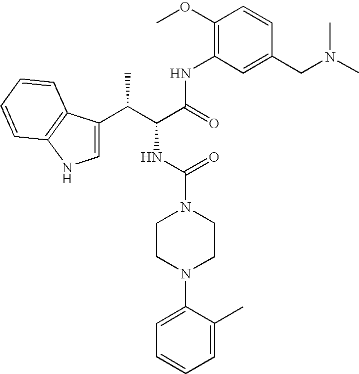

¹H NMR (300 MHz, CDCl₃) δ ppm: 1.58 (d, J=7.2 Hz, 3 H), 1.69 (s, 1 H), 2.20 (s, 6 H), 2.31 (s, 3 H), 2.88 (t, J=4.5 Hz, 4 H), 3.31 (s, 2 H) 3.46-3.61 (m, 7 H), 4.95 (t, J=8.1 Hz, 1 H), 5.41 (d, J=8.1 Hz, 1 H), 6.66 (d, J=8.3 Hz, 1 H), 6.91-7.04 (m, 3 H), 7.07-7.21 (m, 5 H), 7.33 (d, J=7.5 Hz, 1 H), 7.72 (s, 1 H), 7.80 (d, J=7.5 Hz, 1 H), 8.07 (s, 1 H), 8.13 (d, J=1.9 Hz, 1 H).

LC/MS (ESI) m/z 583 (M+H⁺).

### Example 64

### N-((1R,2S)-1-(((5-((dimethylamino)methyl)-2-ethoxyphenyl)amino)carbonyl)-2-(1H-indol-3-yl)propyl)-4-(2-methylphenyl)-1-piperazinecarboxamide

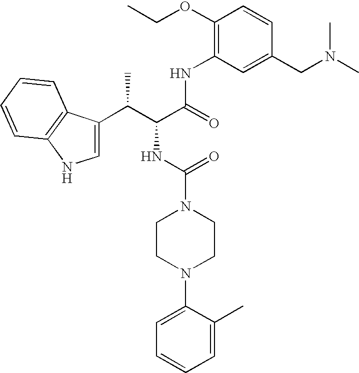

LC/MS (ESI) m/z 597 (M+H⁺).

### Example 65

### N-((1R,2S)-1-(((3-((dimethylamino)methyl)phenyl)amino)carbonyl)-2-(1H-indol-3-yl)propyl)-4-(4-fluorophenyl)-1-piperazinecarboxamide

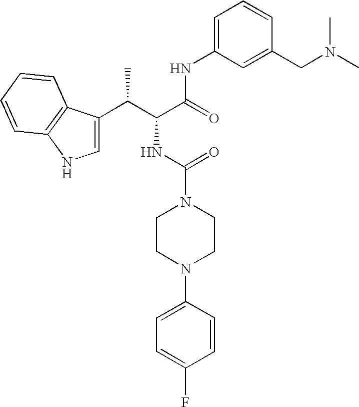

LC/MS (ESI) m/z 557 (M+H⁺).

### Example 66

### N-((1R,2S)-1-(((5-((dimethylamino)methyl)-2-methoxyphenyl)amino)carbonyl)-2-(1H-indol-3-yl)propyl)-4-(4-fluorophenyl)-1-piperazinecarboxamide

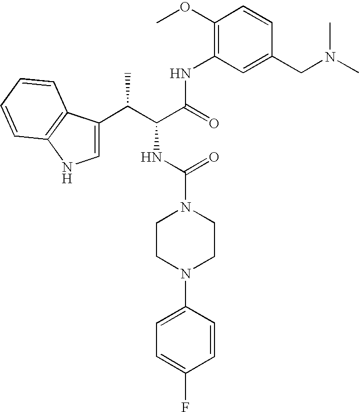

LC/MS (ESI) m/z 587 (M+H⁺).

### Example 67

### N-((1R,2S)-1-(((5-((dimethylamino)methyl)-2-ethoxyphenyl)amino)carbonyl)-2-(1H-indol-3-yl)propyl)-4-(4-fluorophenyl)-1-piperazinecarboxamide

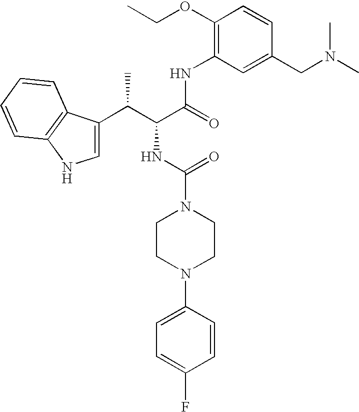

¹H NMR (300 MHz, CDCl₃) δ ppm: 1.23 (t, J=7.0 Hz, 3 H), 1.58 (d, J=7.4 Hz, 3 H), 2.39 (s, 6 H), 3.00-3.09 (m, 4 H), 3.43 -3.66 (m, 6 H), 3.79-3.93 (m, 2 H), 4.86 (t, J=7.5 Hz, 1 H), 5.35 (d, J=7.7 Hz, 1 H), 6.71 (d, J=8.3 Hz, 1 H), 6.84-6.90 (m, 2 H), 6.95-7.01 (m, 3 H), 7.05-7.17 (m, 3 H), 7.32 (d, J=7.9 Hz, 4 H), 7.73 (d, J=7.7 Hz, 1 H), 7.88 (s, 1 H), 8.19-8.20 (m, 2 H).

LC/MS (ESI) m/z 601 (M+H⁺).

### Example 68

### N-((1R,2S)-1-(((2-(dimethylamino)-5-((dimethylamino)methyl)phenyl)amino)carbonyl)-2-(1H-indol-3-yl)propyl)-4-phenyl-1-piperidinecarboxamide

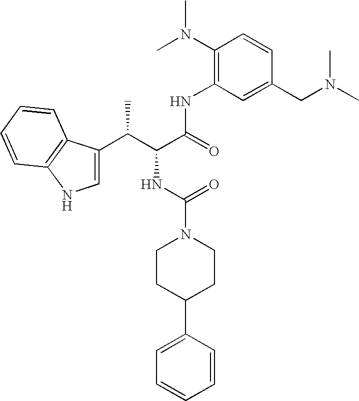

LC/MS (ESI) m/z 581 (M+H⁺).

### Example 69

### N-((1S,2R)-1-(((3-((dimethylamino)methyl)phenyl)amino)carbonyl)-2-(1H-indol-3-yl)propyl)-4-phenyl-1-piperidinecarboxamide

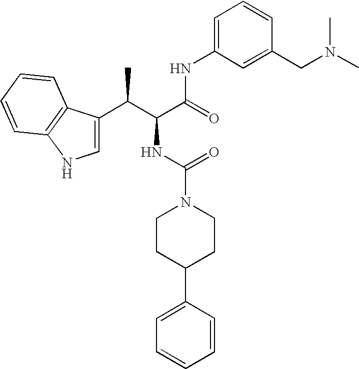

LC/MS (ESI) m/z 538 (M+H⁺).

### Example 70

### N-((1S,2R)-1-(((5-((dimethylamino)methyl)-2-ethoxyphenyl)amino)carbonyl)-2-(1H-indol-3-yl)propyl)-4-phenyl-1-piperidinecarboxamide

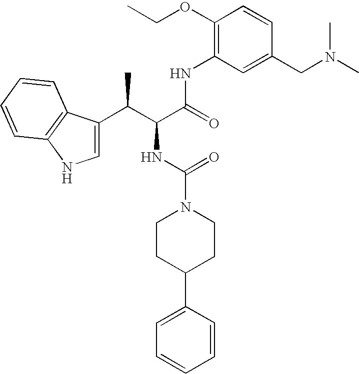

LC/MS (ESI) m/z 582 (M+H⁺).

### Example 71

### N-((1R,2S)-1-(((5-((dimethylamino)methyl)-2-(trifluoromethyl)phenyl)amino)carbonyl)-2-(1H-indol-3-yl)propyl)-4-phenyl-1-piperidinecarboxamide

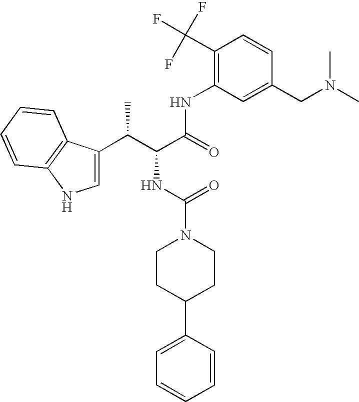

LC/MS (ESI) m/z 606 (M+H⁺).

### Example 72

### N-((1R,2S)-1-(((5-((dimethylamino)methyl)-2-(1-pyrrolidinyl)phenyl)amino)carbonyl)-2-(1H-indol-3-yl)propyl)-4-phenyl-1-piperidinecarboxamide

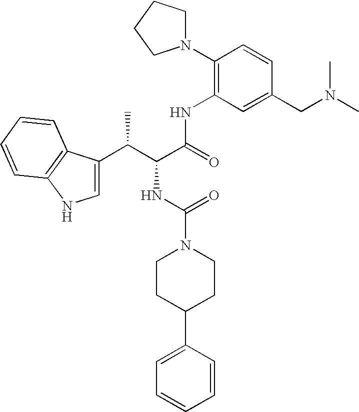

LC/MS (ESI) m/z 607 (M+H⁺).

### Example 73

### N-((1R,2S)-1-(((5-((dimethylamino)methyl)-2-methoxyphenyl)amino)carbonyl)-2-(1H-indol-3-yl)propyl)-4-phenyl-1-piperazinecarboxamide

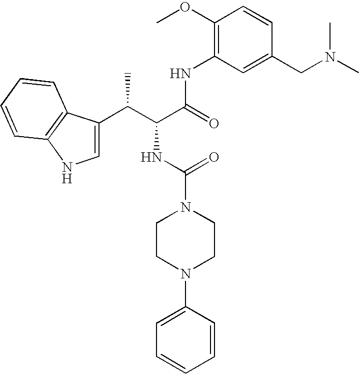

LC/MS (ESI) m/z 569 (M+H⁺).

### Example 74

### N-((1R,2S)-1-(((5-((dimethylamino)methyl)-2-ethoxyphenyl)amino)carbonyl)-2-(1H-indol-3-yl)propyl)-4-phenyl-1-piperazinecarboxamide

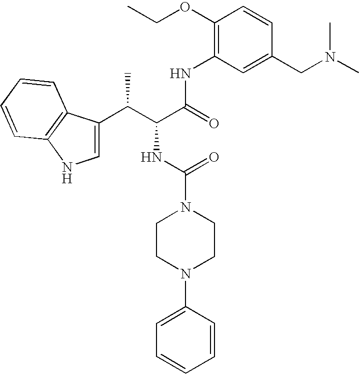

LC/MS (ESI) m/z 583 (M+H⁺).

### Example 75

### N-((1R,2S)-1-(((5-((dimethylamino)methyl)-2-methoxyphenyl)amino)carbonyl)-2-(1H-indol-3-yl)propyl)-4-(2-methylphenyl)-1-piperidinecarboxamide

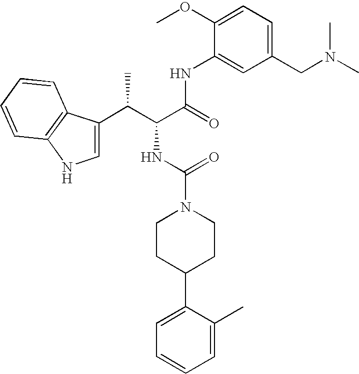

LC/MS (ESI) m/z 582 (M+H⁺).

### Example 76

### N-((1R,2S)-1-(((5-((dimethylamino)methyl)-2-methoxyphenyl)amino)carbonyl)-2-(1H-indol-3-yl)propyl)-4-hydroxy-4-phenyl-1-piperidinecarboxamide

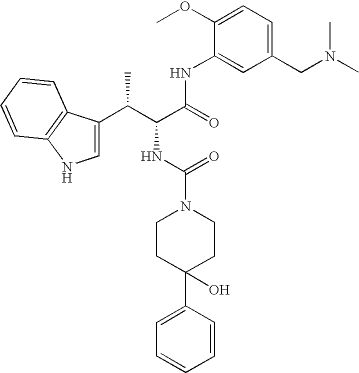

LC/MS (ESI) m/z 584 (M+H⁺).

### Example 77

### 4-(4-chlorophenyl)-N-((1R,2S)-1-(((5-((dimethylamino)methyl)-2-methoxyphenyl)amino)carbonyl)-2-(1H-indol-3-yl)propyl)-4-hydroxy-1-piperidinecarboxamide

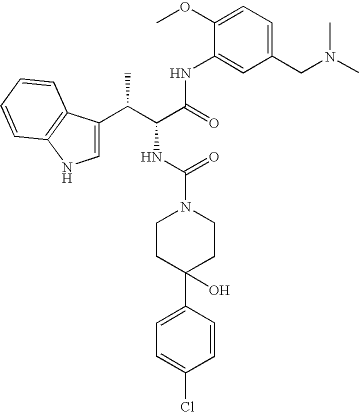

LC/MS (ESI) m/z 618 (M+H⁺).

### Example 78

### N-((1R,2S)-1-(((5-((dimethylamino)methyl)-2-ethoxyphenyl)amino)carbonyl)-2-(1H-indol-3-yl)propyl)-4-(2-methylphenyl)-1-piperidinecarboxamide

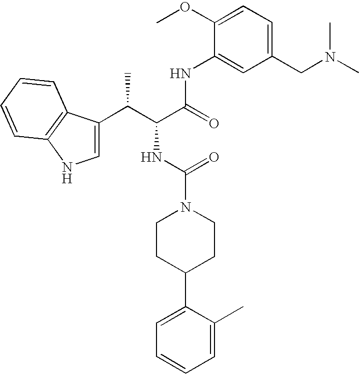

LC/MS (ESI) m/z 596 (M+H⁺).

### Example 79

### N-((1R,2S)-1-(((5-((dimethylamino)methyl)-2-isopropoxyphenyl)amino)carbonyl)-2-(1H-indol-3-yl)propyl)-4-(2-methylphenyl)-1-piperidinecarboxamide

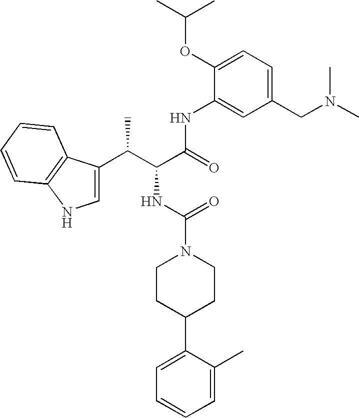

LC/MS (ESI) m/z 610 (M+H⁺).

### Example 80

### N-((1R,2S)-1-(((5-((dimethylamino)methyl)-2-ethylphenyl)amino)carbonyl)-2-(1H-indol-3-yl)propyl)-4-(2-methylphenyl)-1-piperidinecarboxamide

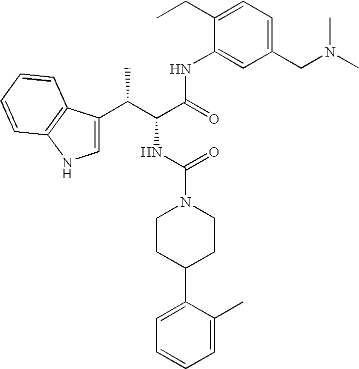

LC/MS (ESI) m/z 580 (M+H⁺).

### Example 81

### 4-cyclohexyl-N-((1R,2S)-1-(((5-((dimethylamino)methyl)-2-methoxyphenyl)amino)carbonyl)-2-(1H-indol-3-yl)propyl)-1-piperazinecarboxamide

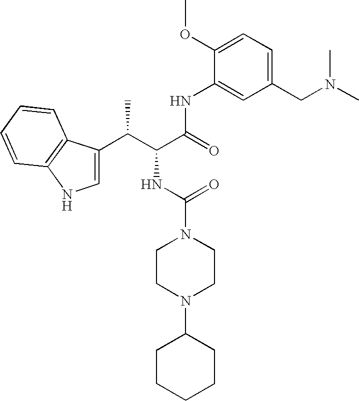

LC/MS (ESI) m/z 575 (M+H⁺).

### Example 82

### 4-cyclohexyl-N-((1R,2S)-1-(((5-((dimethylamino)methyl)-2-ethoxyphenyl)amino)carbonyl)-2-(1H-indol-3-yl)propyl)-1-piperazinecarboxamide

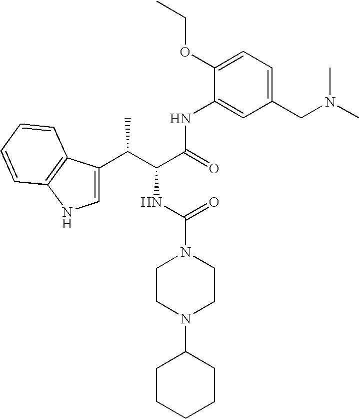

¹H NMR (300 MHz, CDCl₃) δ ppm: 1.01-1.30 (m, 6 H), 1.22 (t, J=6.97 Hz, 3 H), 1.56 (d, J=7.35 Hz, 3 H), 1.74-1.90 (m, 4 H), 2.20 (s, 6 H), 2.21-2.29 (m, 1 H), 2.42-2.57 (m, 4 H), 3.24-3.46 (m, 6 H), 3.55-3.69 (m, 1 H), 3.74-3.96 (m, 2 H), 4.87 (t, J=7.44 Hz, 1 H), 5.27 (d, J=7.72 Hz, 1 H), 6.69 (d, J=8.29 Hz, 1 H), 6.92 (dd, J=8.29, 2.07 Hz, 1 H), 7.01-7.20 (m, 3 H), 7.31 (d, J=7.91 Hz, 1 H), 7.74 (d, J=7.72 Hz, 1 H), 7.92 (s, 1 H) 8.04 (s, 1 H), 8.17 (d, J=1.88 Hz, 1 H).

LC/MS (ESI) m/z 589 (M+H⁺).

### Example 83

### N-((1R,2S)-1-(((3-((dimethylamino)methyl)phenyl)amino)carbonyl)-2-(1H-indol-3-yl)propyl)-4-(2-(trifluoromethyl)phenyl)-1-piperidinecarboxamide

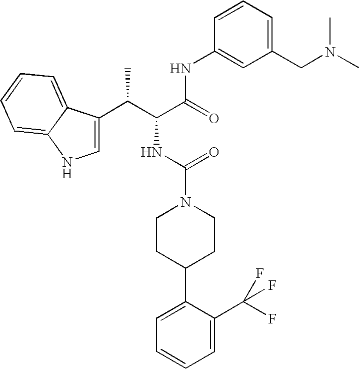

¹H NMR (400 MHz, CDCl₃) δ ppm: 1.58 (d, J=8.1 Hz, 3H), 1.66-1.78 (m, 4H), 2.20 (s, 6H), 2.85-2.96 (m, 2H), 3.06-3.09 (m, 1H), 3.30 (s, 2H), 3.54-3.62 (m, 1H), 4.02-4.17 (m, 2H), 4.85 (t, J=8.3 Hz, 1H), 5.36 (d, J=8.1 Hz, 1H), 6.96-7.37 (m, 9H), 7.48-7.52 (m, 2H), 7.63 (d, J=7.8 Hz, 1H), 7.81 (d, J=7.8 Hz, 1H), 8.19 (s, 1H).

LC/MS (ESI) m/z: 606 (M+H⁺)

### Example 84

### N-((1R,2S)-1-(((3-((dimethylamino)methyl)phenyl)amino)carbonyl)-2-(1H-indol-3-yl)propyl)-4-(2-methoxyphenyl)-1-piperidinecarboxamide

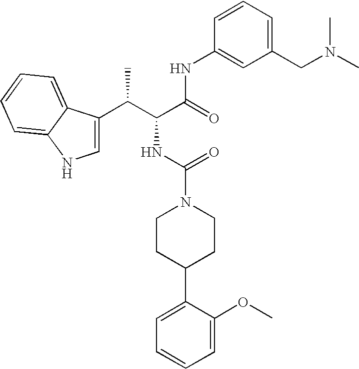

¹H-NMR (400 MHz, CDCl₃) δ ppm: 1.50 (d, J=7.3 Hz, 3H), 1.57 (s, 6H), 1.89-2.09 (m, 4H), 2.60 (s, 3H), 2.58-2.64 (m, 1H) 3.26-3.36 (m, 2H), 3.58-3.83 (m, 3H), 3.62 (s, 2H), 4.84 (dd, J=8.3, 5.4 Hz, 1H), 5.04 (d, J=8.3 Hz, 1H), 7.04-7.37 (m, 13H), 7.63 (d, J=7.8 Hz, 1H) , 8.10 (s, 1H).

LC/MS (ESI) m/z: 568 (M+H⁺)

### Example 85

### N-((1R,2S)-1-(((3-((dimethylamino)methyl)phenyl)amino)carbonyl)-2-(1H-indol-3-yl)propyl)-4-hydroxy-4-(2-methylphenyl)-1-piperidinecarboxamide

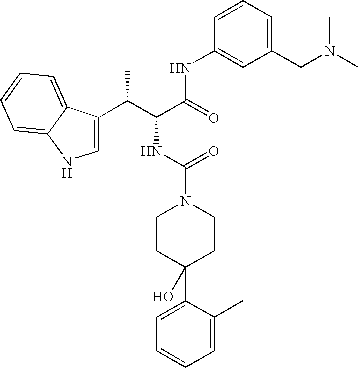

¹H NMR (400 MHz, CDCl₃) δ ppm: 1.56 (d, J=7.1 Hz, 3H), 1.92-2.14 (m, 4H), 2.18 (s, 6H), 2.60 (s, 3H), 3.16-3.42 (m, 5H), 3.55-3.62 (m, 1H), 3.86 (dd, J=44.6, 12.8 Hz, 2H), 4.83 (t, J=8.3 Hz, 1H), 5.47 (d, J=7.8 Hz, 1H), 6.94-7.26 (m, 9H), 7.31-7.35 (m, 2H), 7.62 (s, 1H), 7.78 (d, J=7.8 Hz, 2H), 8.32 (s, 1H).

LC/MS (ESI) m/z: 568 (M+H⁺)

### Example 86

### N-((1R,2S)-1-(((3-((dimethylamino)methyl)phenyl)amino)carbonyl)-2-(1H-indol-3-yl)propyl)-4-(2-ethylphenyl)-1-piperidinecarboxamide

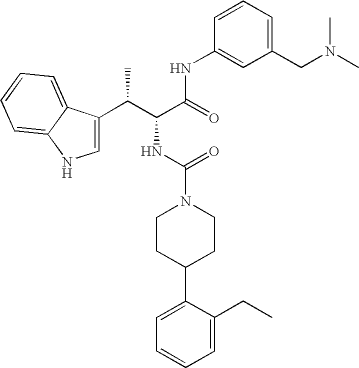

¹H-NMR (400 MHz, CDCl₃) δ ppm: 1.22 (t, J=7.6 Hz, 3H), 1.58 (d, J=7.1 Hz, 3H), 1.61-1.72 (m, 4H), 2.20 (s, 6H), 2.69 (q, J=7.4 Hz, 2H), 2.82-2.96 (m, 3H), 3.30 (s, 2H), 3.58-3.63 (m, 1H), 4.02-4.19 (m, 2H), 4.84 (t, J=8.2 Hz, 1H), 5.42 (d, J=7.8 Hz, 1H), 6.95-7.21 (m, 11H), 7.35 (d, J=8.1 Hz, 1H), 7.61 (s, 1H), 7.80 (d, J=8.1 Hz, 1H), 8.30 (s, 1H).

LC/MS (ESI) m/z: 566 (M+H⁺)

### Example 87

### N-((1R,2S)-1-(((3-((dimethylamino)methyl)phenyl)amino)carbonyl)-2-(1H-indol-3-yl)propyl)-4-hydroxy-4-(1,3-thiazol-2-yl)-1-piperidinecarboxamide

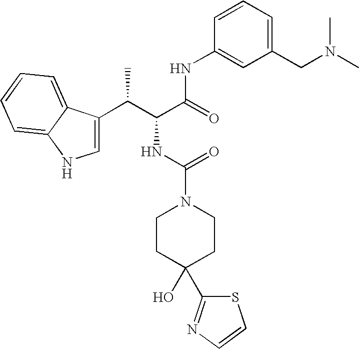

¹H NMR (400 MHz, DMSO-d₆) δ ppm: 1.33 (d, J=7.1 Hz, 3H), 1.64-2.03 (m, 4H), 3.07-3.16 (m, 2H), 3.36 (br, 1H), 3.51-3.59 (m, 3H), 4.45 (t, J=7.4 Hz, 1H), 6.08 (br, 1H), 6.38 (d, J=8.4 Hz, 1H), 6.95-7.06 (m, 2H), 7.15 (d, J=2.0 Hz, 1H), 7.33 (d, J=7.8 Hz, 1H), 7.54 (d, J=8.4 Hz, 1H), 7.59 (d, J=3.3 Hz, 1H), 7.72 (d, J=3.3 Hz, 1H), 10.82 (s, 1H).

LC/MS (ESI) m/z: 429 (M+H⁺)

### Example 88

### N-((1R,2S)-1-(((3-((dimethylamino)methyl)phenyl)amino)carbonyl)-2-(1H-indol-3-yl)propyl)-4-(4-methyl-1,3-thiazol-2-yl)-1-piperidinecarboxamide

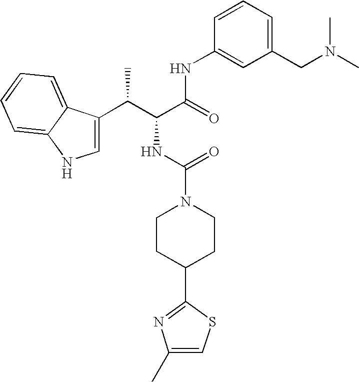

¹H-NMR (400 MHz, CDCl₃) δ ppm: 1.59 (d, J=6.8 Hz, 3H), 1.72-1.85 (m, 2H), 2.17 (s, 6H), 2.04-2.24 (m, 2H), 3.30-3.36 (m, 5H), 3.67-3.72 (m, 1H), 3.82-3.88 (m, 2H), 4.78-4.83 (m, 1H), 5.98 (br, 1H), 6.81-6.87 (m, 2H), 7.00-7.28 (m, 7H), 7.71 (d,-J=3.2 Hz, 1H), 7.77 (d, J=7.8 Hz, 1H), 8.20 (brs 1H), 9.05 (brs, 1H).

LC/MS (ESI) m/z: 561 (M+H⁺)

### Example 89

### N-((1R,2S)-2-(1H-indol-3-yl)-1-(((2-(trifluoroacetyl)-1,2,3,4-tetrahydro-7-isoquinolinyl)amino)carbonyl)propyl)-4-phenyl-1-piperidinecarboxamide

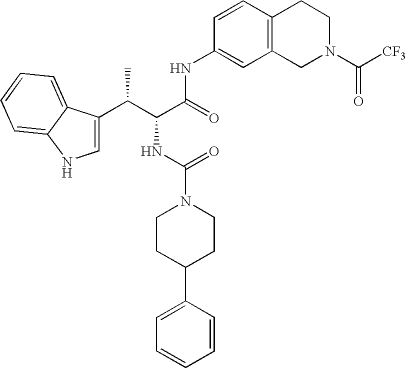

A mixed solution of (2R,3S)-3-(1H-indol-3-yl)-2-(((4-phenyl-1-piperidinyl)carbonyl)amino)butanoic acid (304 mg), 2-(trifluoroacetyl)-1,2,3,4-tetrahydro-7-isoquinolinamine (105.7 mg), WSC (220 mg) and HOBt (150 mg) in acetonitrile (2 mL)-THF (2 mL) was stirred at room temperature for 16 hours. The reaction solution was diluted with ethyl acetate (2 mL), and a saturated aqueous solution of sodium carbonate (2 mL)-water (2 mL) was added and the mixture was extracted. The extract was purified by column chromatography (aminopropyl silica gel: 5 g, developing solvent: ethyl acetate). The obtained residue was purified by Mega Bond Elute (product name, SiO₂, 5 g, developing solvent: hexane/ethyl acetate=10/1 to 1/1) to give the title compound as a white amorphous powder (350 mg, yield 79%).

LC/MS (ESI) m/z 632 (M+H⁺).

The following compounds mentioned in Examples 90 and 91 were synthesized according to the same method as Example 89.

### Example 90

### N-((1R,2S)-2-(1H-indol-3-yl)-1-(((2-(trifluoroacetyl)-1,2,3,4-tetrahydro-7-isoquinolinyl)amino)carbonyl)propyl)-4-(2-methylphenyl)-1-piperidinecarboxamide

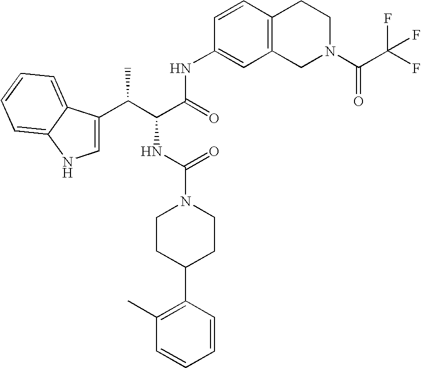

LC/MS (ESI) m/z: 646 (M+H⁺)

### Example 91

### 4-(4-fluorophenyl)-N-((1R,2S)-2-(1H-indol-3-yl)-1-(((2-(trifluoroacetyl)-1,2,3,4-tetrahydro-7-isoquinolinyl)amino)carbonyl)propyl)-1-piperidinecarboxamide

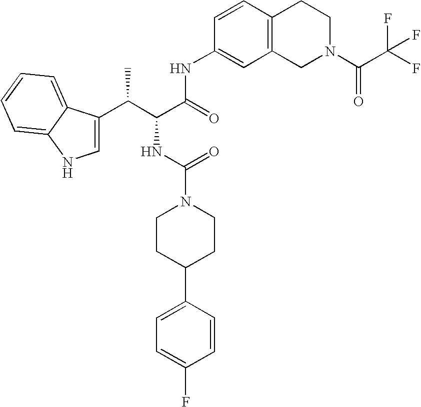

LC/MS (ESI) m/z: 650 (M+H⁺)

### Example 92

### N-((1R,2S)-2-(1H-indol-3-yl)-1-((1,2,3,4-tetrahydro-7-isoquinolinylamino)carbonyl)propyl)-4-phenyl-1-piperidinecarboxamide

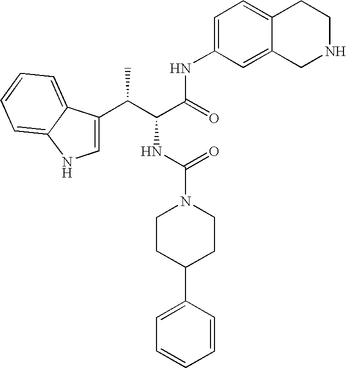

An aqueous solution of 10% potassium carbonate (6 mL) was added to a solution of N-((1R,2S)-2-(1H-indol-3-yl)-1-(((2-(trifluoroacetyl)-1,2,3,4-tetrahydro-7-isoquinolinyl)amino)carbonyl)propyl)-4-phenyl-1-piperidinecarboxamide (304 mg) in methanol (15 mL) at room temperature and, the mixture was stirred for 16 hours. After the completion of the reaction, methanol was removed by evaporation. Water was added to the residue, and the precipitates were collected by filtration, washed with water and dried to give the title compound as a white powder (282 mg, yield 95%).

LC/MS (ESI) m/z 536 (M+H⁺).

The following compounds mentioned in Examples 93 and 94 were synthesized according to the same method as Example 92.

### Example 93

### N-((1R,2S)-2-(1H-indol-3-yl)-1-((1,2,3,4-tetrahydro-7-isoquinolinylamino)carbonyl)propyl)-4-(2-methylphenyl)-1-piperidinecarboxamide

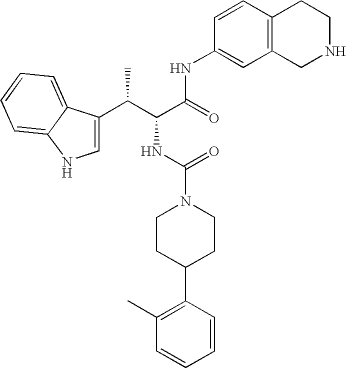

¹H-NMR (400 MHz, CDCl₃) δ ppm: 1.57 (d, J=7.1 Hz, 3H), 1.62-1.76 (m, 2H), 2.14-2.19 (m, 2H), 2.34 (s, 3H), 2.66 (t, J=5.6 Hz, 2H), 2.82-2.93 (m, 3H), 3.00-3.11 (m, 3H), 3.51-3.59 (m, 1H), 3.75-3.84 (m, 2H), 4.03 (dt, J=13.0 Hz, 1H), 4.17 (d, J=13.0 Hz, 1H), 4.87 (t, J=8.3 Hz, 1H), 5.43 (d, J=8.3 Hz, 1H), 6.71-6.73 (m, 2H), 6.84 (d, J=8.5 Hz, 1H), 7.08-7.21 (m, 7H), 7.35 (d, J=7.8 Hz, 1H), 7.78-7.80 (m, 2H), 8.33 (br, 1H).

LC/MS (ESI) m/z: 550 (M+H⁺)

### Example 94

### 4-(4-fluorophenyl)-N-((1R,2S)-2-(1H-indol-3-yl)-1-((1,2,3,4-tetrahydro-7-isoquinolinylamino)carbonyl)propyl)-1-piperidinecarboxamide

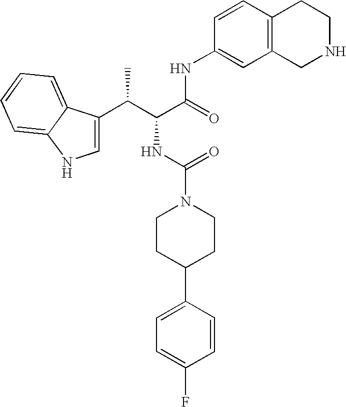

¹H-NMR (400 MHz, CDCl₃) δ ppm: 1.48-1.83 (m, 4H), 1.57 (d, J=7.3 Hz, 3H), 2.61-2.68 (m, 3H), 2.81-2.94 (m, 3H), 3.06 (t, J=6.0 Hz, 2H), 3.47-3.55 (m, 1H), 3.84 (s, 2H), 4.01-4.47 (m, 2H), 4.84 (t, J=8.3 Hz, 1H), 5.36 (d, J=8.3 Hz, 1H), 6.67-6.72 (m, 2H), 6.87 (d, J=8.1 Hz, 1H), 6.96-7.02 (m, 2H), 7.10-7.30 (m, 5H), 7.36 (d, J=8.1 Hz, 2H), 7.80 (d, J=7.8 Hz, 1H), 8.10 (s, 1H).

LC/MS (ESI) m/z: 554 (M+H⁺)

### Example 95

### N-((1R,2S)-1-(((5-(2-aminoethyl)-2-methoxyphenyl)amino)carbonyl)-2-(1H-indol-3-yl)propyl)-4-phenyl-1-piperidinecarboxamide

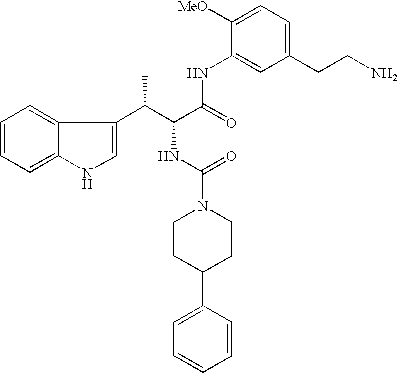

A mixed solution of (2R,3S)-3-(1H-indol-3-yl)-2-(((4-phenyl-1-piperidinyl)carbonyl)amino)butanoic acid (164 mg), N-[2-(3-amino-4-methoxyphenyl)ethyl]-2,2,2-trifluoroacetamide (98 mg), WSC (102 mg) and HOBt (86 mg) in acetonitrile (0.8 mL)-THF (0.8 mL) was stirred at room temperature for 2 days. The reaction solution was added to a saturated aqueous solution of sodium hydrogen carbonate and extracted with ethyl acetate. The extract was filtered through a silica gel layer and concentrated.

The residue was dissolved in THF (6.0 mL) and an aqueous solution of 10% potassium carbonate (2.0 mL) was added. The mixture was stirred at room temperature overnight. The reaction solution was concentrated and the organic solvent was removed by evaporation. The obtained precipitates were collected by filtration, washed with water and dried to give the title compound (205 mg, yield 93%).

¹H-NMR (300 MHz, CDCl₃) δ ppm: 1.43-1.75 (m, 4 H), 1.57 (d, J=7.2 Hz, 3 H) 1.78-1.90 (m, 2 H) 2.58-2.73 (m, 3 H) 2.80-2.97 (m, 4 H) 3.50-3.65 (m, 1 H) 3.54 (s, 3 H) 4.01-4.19 (m, 2 H) 4.94 (t, J=7.9 Hz, 1 H) 5.33-5.43 (m, 1 H) 6.63 (d, J=8.3 Hz, 1 H) 6.75-6.80 (m, 1 H) 7.03-7.25 (m, 6 H) 7.31 (t, J=7.3 Hz, 3 H) 7.77-7.85 (m, 2 H) 8.07 (s, 1 H) 8.19 (s, 1 H).

LC/MS (ESI) m/z: 554 (M+H⁺)

### Example 96

### N-((1R,2S)-1-(((5-(2-aminoethyl)-2-methoxyphenyl)amino)carbonyl)-2-(1H-indol-3-yl)propyl)-4-(2-methylphenyl)-1-piperidinecarboxamide

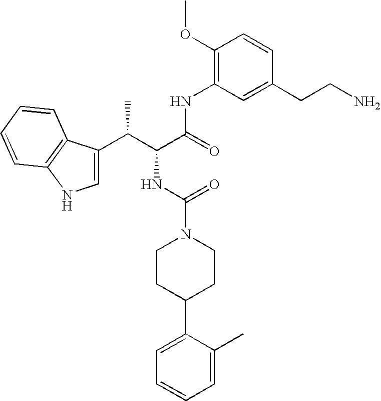

The title compound was obtained by the same method as Example 95.

LC/MS (ESI) m/z: 568 (M+H⁺)

The compounds described in the following Examples 97-100 were produced in the similar manner as in Example 1.

### Example 97

### tert-butyl {3-[((2R,3S)-3-(1H-indol-3-yl)-2-{[(4-phenylpiperidin-1-yl)carbonyllamino}butanoyl)amino]benzyl}methylcarbamate

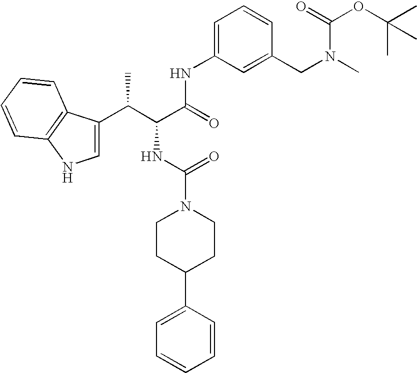

LC/MS (ESI) m/z 624 (M+H⁺).

### Example 98

### tert-butyl cyclopropyl{3-[((2R,3S)-3-(1H-indol-3-yl)-2-{[(4-phenylpiperidin-1-yl)carbonyl]amino}butanoyl)amino]benzyl}carbamate

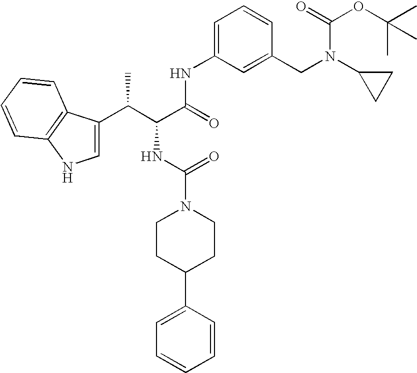

LC/MS (ESI) m/z 650 (M+H⁺).

### Example 99

### N-[(1R,2S)-1-[({5-[(dimethylamino)methyl]-2-methoxyphenyl}amino)carbonyl]-2-(1H-indol-3-yl)propyl]-4-(4-fluorophenoxy)piperidine-1-carboxamide

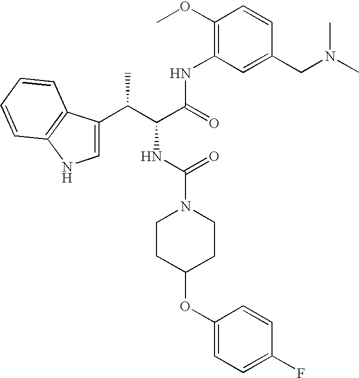

LC/MS (ESI) m/z 602 (M+H⁺).

### Example 100

### N-[(1R,2S)-1-[({5-[(dimethylamino)methyl]-2-ethoxyphenyl}amino)carbonyl]-2-(1H-indol-3-yl)propyl]-4-(4-fluorophenoxy)piperidine-1-carboxamide

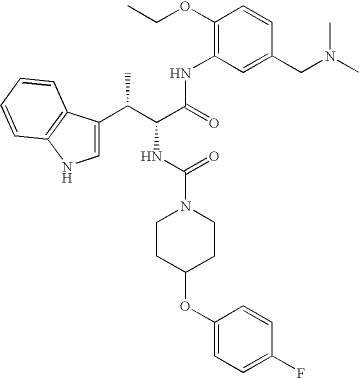

LC/MS (ESI) m/z 616 (M+H⁺).

### Example 101

### N-{(1R,2S)-2-(1H-indol-3-yl)-1-[(2,3,4,5-tetrahydro-1H-3-benzazepin-7-ylamino)carbonyl]propyl}-4-phenylpiperidine-1-carboxamide

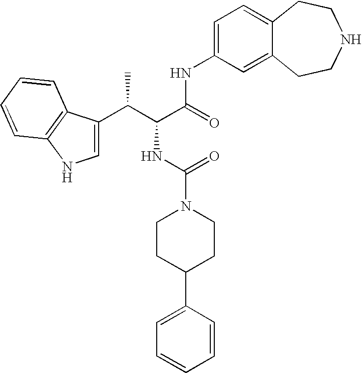

A mixed solution of (2R,3S)-3-(1H-indol-3-yl)-2-{[(4-phenylpiperidin-1-yl)carbonyl]amino}butanoic acid (426 mg), 3-(trifluoroacetyl)-2,3,4,5-tetrahydro-1H-3-benzazepin-7-amine (258 mg), WSC (297 mg) and HOBt (207 mg) in acetonitrile (2 mL)-THF (2 mL) was stirred at room temperature for 16 hrs. A saturated solution of sodium carbonate was added to the reaction solution and extracted with ethyl acetate. The extract was dried (MgSO₄) and the solvent was evaporated under reduced pressure. The residue was purified by silica gel column chromatography (developing solvent: hexane/ethyl acetate=7/3-2/3-1/4). To a solution of the obtained residue in methanol (25 mL) was added 10% aqueous potassium carbonate solution (9 mL) at room temperature, and the mixture was stirred for 16 hrs. After the completion of the reaction, methanol was evaporated. Water was added to the residue, the resulting precipitates were collected by filtration, washed with water and dried to give the title compound (339 mg, yield 62%) as a white powder.

LC/MS (ESI) m/z 550 (M+H⁺).

The compounds described in the following Examples 102-107 were produced in the similar manner as in Example 101.

### Example 102

### N-{(1R,2S)-2-(1H-indol-3-yl)-1-[(1,2,3,4-tetrahydroisoquinolin-7-ylamino)carbonyl]propyl}-4-phenylpiperazine-1-carboxamide

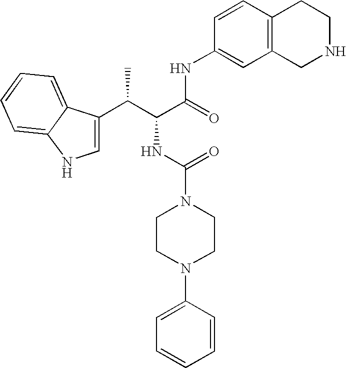

LC/MS (ESI) m/z 537 (M+H⁺).

### Example 103

### 4-benzoyl-N-{(1R,2S)-2-(1H-indol-3-yl)-1-[(1,2,3,4-tetrahydroisoquinolin-7-ylamino)carbonyl]propyl}piperazine-1-carboxamide

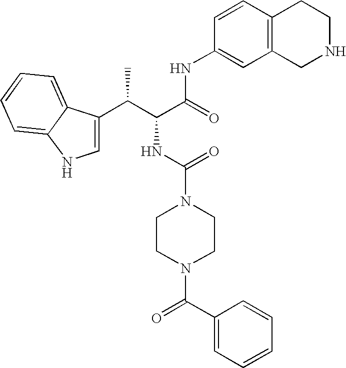

LC/MS (ESI) m/z 565 (M+H⁺).

### Example 104

### 1-benzoyl-N-{(1R,2S)-2-(1H-indol-3-yl)-1-[(1,2,3,4-tetrahydroisoquinolin-7-ylamino)carbonyl]propyl}piperidine-4-carboxamide

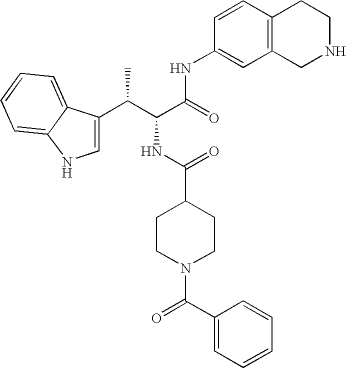

LC/MS (ESI) m/z 564 (M+H⁺).

### Example 105

### 4-(4-fluorophenoxy)-N-{(1R,2S)-2-(1H-indol-3-yl)-1-[(1,2,3,4-tetrahydroisoquinolin-7-ylamino)carbonyl]propyl}piperidine-1-carboxamide

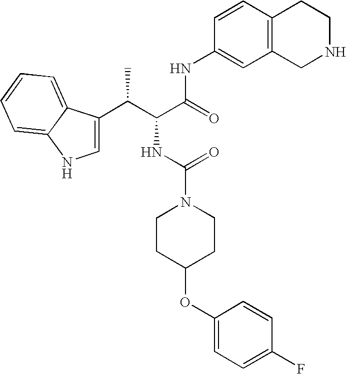

LC/MS (ESI) m/z 570 (M+H⁺).

### Example 106

### N-{(1R,2S)-2-(1H-indol-3-yl)-1-[(1,2,3,4-tetrahydroisoquinolin-7-ylamino)carbonyl]propyl}-4-phenylcyclohexanecarboxamide

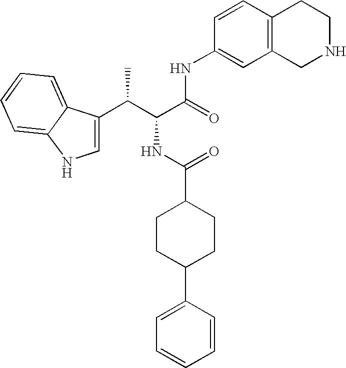

LC/MS (ESI) m/z 535 (M+H⁺).

### Example 107

### N-[(1R,2S)-1-[(2,3-dihydro-1H-isoindol-5-ylamino)carbonyl]-2-(1H-indol-3-yl)propyl]-4-phenylpiperidine-1-carboxamide

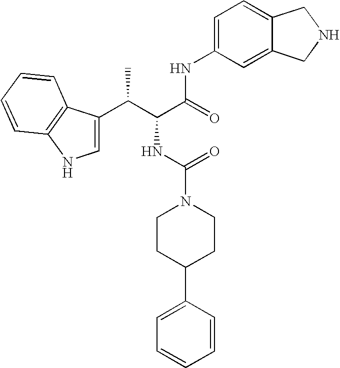

LC/MS (ESI) m/z 522 (M+H⁺).

### Example 108

### N-((1R,2S)-2-(1H-indol-3-yl)-1-{[(2-methyl-1,2,3,4-tetrahydroisoquinolin-7-yl)amino]carbonyl}propyl)-4-phenylpiperidine-1-carboxamide

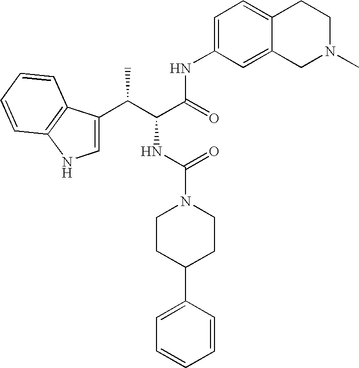

To a solution of N-{(1R,2S)-2-(1H-indol-3-yl)-1-[(1,2,3,4-tetrahydroisoquinolin-7-ylamino)carbonyl]propyl}-4-phenylpiperidine-1-carboxamide (134 mg) in ethanol (1.5 mL) was added 30% aqueous solution of formaldehyde (24 mg) at room temperature, and the mixture was stirred for 10 min. Then sodium triacetoxyborohydride (64 mg) was added at room temperature, and the mixture was stirred for 1 hr. After the completion of the reaction, the reaction mixture was poured into saturated aqueous solution of sodium hydrogen carbonate and extracted with ethyl acetate. The extract was filtered by passing through a silica gel layer and concentrated. The obtained residue was washed with diisopropyl ether-hexane and dried to give the title compound (114 mg, yield 83%).

LC/MS (ESI) m/z 550 (M+H⁺).

The compounds described in the following Examples 109-115 were produced in the similar manner as in Example 108.

### Example 109

### N-((1R,2S)-2-(1H-indol-3-yl)-1-{[(2-methyl-1,2,3,4-tetrahydroisoquinolin-7-yl)amino]carbonyl}propyl)-4-(2-methylphenyl)piperidine-1-carboxamide

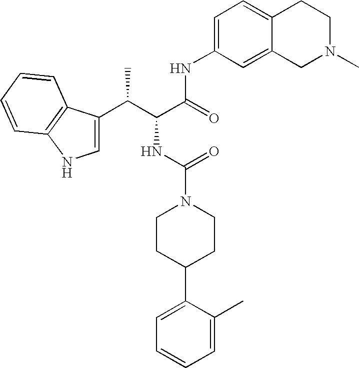

LC/MS (ESI) m/z 564 (M+H⁺).

### Example 110

### N-((1R,2S)-2-(1H-indol-3-yl)-1-{[(2-methyl-1,2,3,4-tetrahydroisoquinolin-7-yl)amino]carbonyl}propyl)-4-phenylpiperazine-1-carboxamide

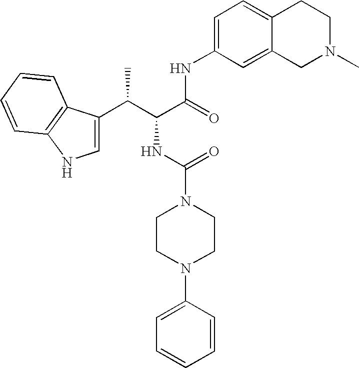

LC/MS (ESI) m/z 551 (M+H⁺).

### Example 111

### 4-benzoyl-N-((1R,2S)-2-(1H-indol-3-yl)-1-{[(2-methyl-1,2,3,4-tetrahydroisoquinolin-7-yl)amino]carbonyl}propyl)piperazine-1-carboxamide

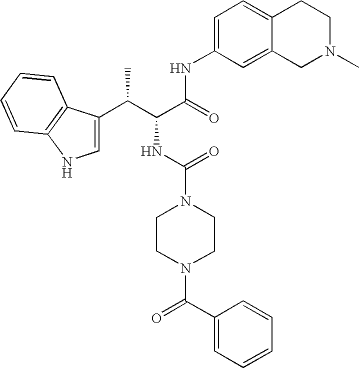

LC/MS (ESI) m/z 579 (M+H⁺).

### Example 112

### 1-benzoyl-N-((1R,2S)-2-(1H-indol-3-yl)-1-{[(2-methyl-1,2,3,4-tetrahydroisoquinolin-7-yl)amino]carbonyl}propyl)piperidine-4-carboxamide

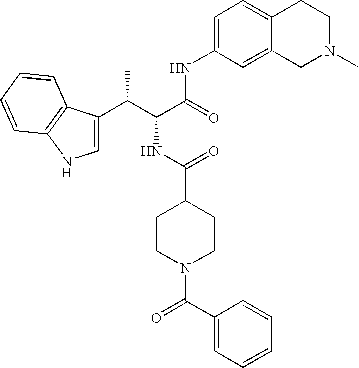

LC/MS (ESI) m/z 578 (M+H⁺).

### Example 113

### 4-(4-fluorophenoxy)-N-((1R,2S)-2-(1H-indol-3-yl)-1-{[(2-methyl-1,2,3,4-tetrahydroisoquinolin-7-yl)amino]carbonyl}propyl)piperidine-1-carboxamide

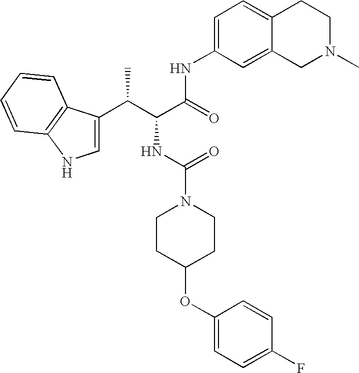

LC/MS (ESI) m/z 584 (M+H⁺).

### Example 114

### N-((1R,2S)-2-(1H-indol-3-yl)-1-{[(3-methyl-2,3,4,5-tetrahydro-1H-3-benzazepin-7-yl)amino]carbonyl}propyl)-4-phenylpiperidine-1-carboxamide

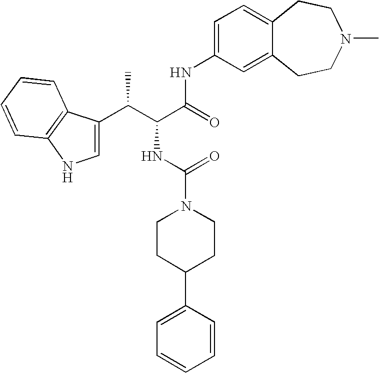

LC/MS (ESI) m/z 564 (M+H⁺).

### Example 115

### N-((1R,2S)-2-(1H-indol-3-yl)-1-{[(2-methyl-2,3-dihydro-1H-isoindol-5-yl)amino]carbonyl}propyl)-4-phenylpiperidine-1-carboxamide

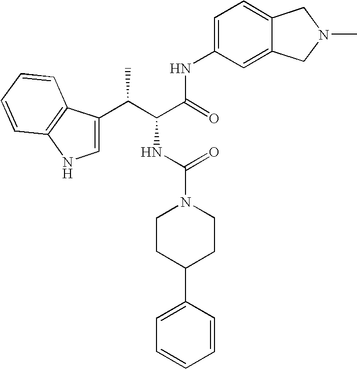

LC/MS (ESI) m/z 536 (M+H⁺).

### Example 116

### N-{(1R,2S)-2-(1H-indol-3-yl)-1-[({3-[(methylamino)methyl]phenyl}amino)carbonyl]propyl}-4-phenylpiperidine-1-carboxamide

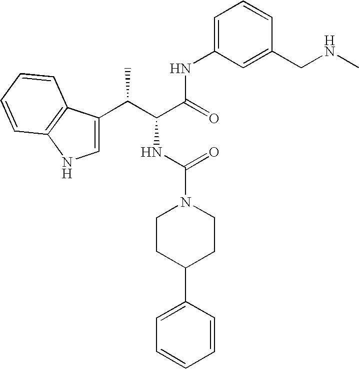

To a solution of tert-butyl {3-[((2R,3S)-3-(1H-indol-3-yl)-2-{[(4-phenylpiperidin-1-yl)carbonyl]amino}butanoyl)amino]benzyl}methylcarbamate (0.63 g) in dioxane (2 mL) was added 4N hydrochloric acid-dioxane solution (1 mL) at room temperature, and the mixture was stirred for 2 hrs. The solvent was evaporated under reduced pressure and the residue was purified by HPLC (acetonitrile/water=10/90-100/0, containing 0.1% trifluoroacetic acid). The obtained fraction was concentrated and the residue was dissolved in acetonitrile. Thereto was added saturated aqueous solution of sodium hydrogen carbonate. The resulting precipitates were collected by filtration and dried under reduced pressure to give the title compound (39.5 mg, yield 7.5%).

LC/MS (ESI) m/z 524 (M+H⁺).

The compound described in the following Example 117 was produced in the similar manner as in Example 116.

### Example 117

### N-[(1R,2S)-1-[({3-[(cyclopropylamino)methyl]phenyl}amino)carbonyl]-2-(1H-indol-3-yl)propyl]-4-phenylpiperidine-1-carboxamide

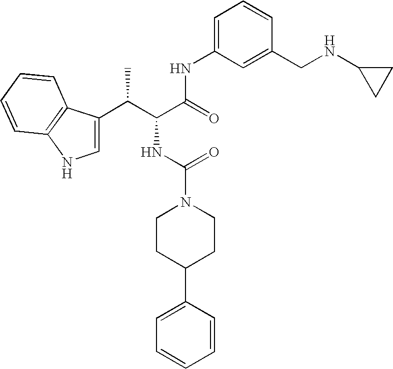

LC/MS (ESI) m/z 550 (M+H⁺).

### Example 118

### N-[(1R,2S)-1-[({3-[(dimethylamino)methyl]phenyl}amino)carbonyl]-2-(1H-indol-3-yl)propyl]-1-phenylpiperidine-4-carboxamide

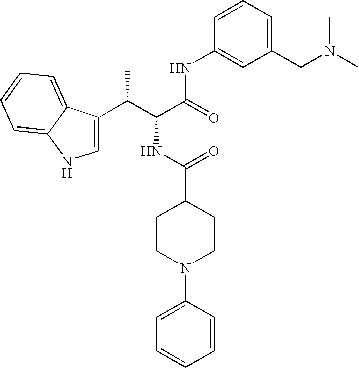

A mixed solution of (2R,3S)-2-amino-N-{3-[(dimethylamino)methyl]phenyl}-3-(1H-indol-3-yl)butanamide dihydrochloride (42 mg), 1-phenylpiperidine-4-carboxylic acid (25 mg), triethylamine (0.033 mL), WSC (35 mg) and HOBt (24 mg) in acetonitrile (0.5 ml)-THF(0.5 mL) was stirred at room temperature for 16 hrs. The reaction solution was diluted with ethyl acetate, saturated solution of sodium carbonate was added and the mixture was subjected to extraction. The extract was purified by column chromatography (aminopropyl silica gel, developing solvent: hexane/ethyl acetate=1/1-1/4). The obtained residue was washed with dichloromethane-diethyl ether to give the title compound (17 mg, yield 27%) as a white powder.

LC/MS (ESI) m/z 538 (M+H⁺).

The compounds described in the following Examples 119-120 were produced in the similar manner as in Example 118.

### Example 119

### trans-N-[(1R,2S)-1-[({3-[(dimethylamino)methyl]phenyl}amino)carbonyl]-2-(1H-indol-3-yl)propyl]-4-phenylcyclohexanecarboxamide

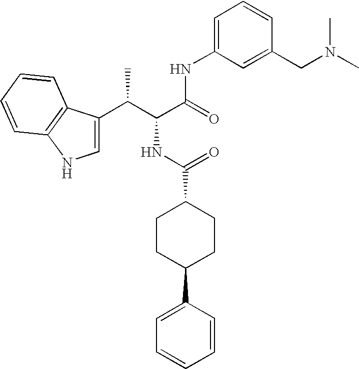

LC/MS (ESI) m/z 537 (M+H⁺).

### Example 120

### N-[(1R,2S)-1-[({3-[(dimethylamino)methyl]phenyl}amino)carbonyl]-2-(1H-indol-3-yl)propyl]biphenyl-4-carboxamide

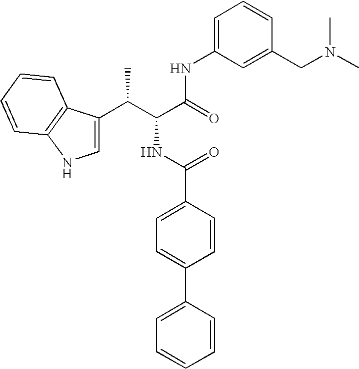

LC/MS (ESI) m/z 531 (M+H⁺).

### Example 121

### (2R,3S)-N-[3-(aminomethyl)phenyl]-2-{[3-(1-benzoylpiperidin-4-yl)propanoyl]amino}-3-(1H-indol-3-yl)butanamide

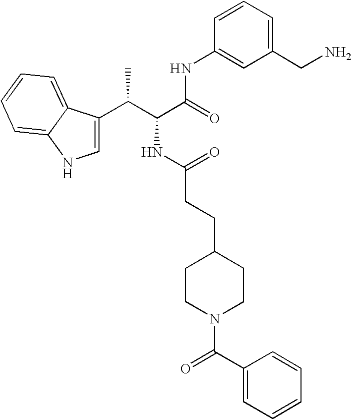

To a mixture of tert-butyl (3-{[(2R,3S)-2-amino-3-(1H-indol-3-yl)butanoyl]amino}benzyl)carbamate (110 mg, 0.26 mmol) and 3-(1-benzoylpiperidin-4-yl)propanoic acid (65 mg), WSC (72 mg) and HOBt (50 mg) were added THF (1.0 mL) and acetonitrile (1.0 mL) at room temperature, and the mixture was stirred for 16 hrs. To the reaction solution were added saturated aqueous solution of sodium hydrogen carbonate and ethyl acetate and the mixture was subjected to extraction. The organic layer was filtered by passing through an aminopropyl silica gel layer and concentrated under reduced pressure to give tert-butyl (3-{[(2R,3S)-2-{[3-(1-benzoylpiperidin-4-yl)propanoyl]amino}-3-(1H-indol-3-yl)butanoyl]amino}benzyl)carbamate (150 mg). The obtained residue (145 mg) was dissolved in dioxane (1.0 mL) and 4N hydrochloric acid-dioxane solution (1.0 mL) was added and the mixture was stirred at room temperature for 30 min. The reaction solution was diluted with ethyl acetate and the resulting precipitates were collected by filtration. The residue was purified by HPLC (acetonitrile/water=10/90-100/0, containing 0.1% trifluoroacetic acid) to give the title compound (47 mg, yield 38%) as a pale yellow powder.

LC/MS (ESI) m/z 566 (M+H⁺).

### Example 122

### (2R,3S)-2-{[3-(1-benzoylpiperidin-4-yl)propanoyl]amino)-N-{3-[(dimethylamino)methyl]phenyl}-3-(1H-indol-3-yl)butanamide

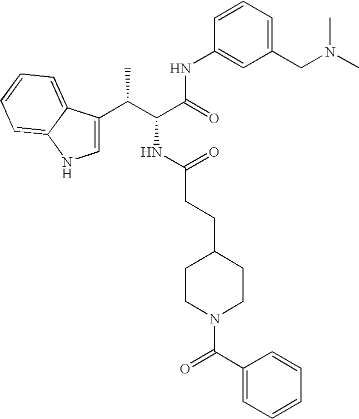

To a mixture of (2R,3S)-N-[3-(aminomethyl)phenyl]-2-{[3-(1-benzoylpiperidin-4-yl)propanoyl]amino}-3-(1H-indol-3-yl)butanamide (27 mg) and 30% aqueous solution of formaldehyde (9 mg) in methanol (0.5 mL) was added sodium triacetoxyborohydride (22 mg) at room temperature, and the mixture was stirred for 4 hrs. To the reaction mixture was added a saturated aqueous solution of sodium hydrogen carbonate (0.5 mL) and the mixture was diluted with water. The resulting precipitates were collected by filtration and dried to give the title compound (20 mg, yield 72%) as a pale yellow powder.

LC/MS (ESI) m/z 594 (M+H⁺).

### Example 123

### (2R,3S)-N-{3-[(dimethylamino)methyl]phenyl}-3-(1H-indol-3-yl)-2-{[(4-phenylcyclohexyl)methyl]amino}butanamide

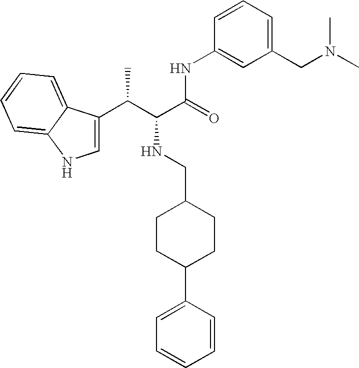

A mixture of (2R,3S)-2-amino-N-{3-[(dimethylamino)methyl]phenyl}-3-(1H-indol-3-yl)butanamide (175 mg), 4-phenylcyclohexanecarbaldehyde (104 mg) and ethanol (2 mL) was stirred at room temperature for 1 hr. Sodium triacetoxyborohydride (127 mg) was added at the similar temperature and the mixture was stirred for 12 hrs. To the reaction solution was added saturated aqueous solution of sodium hydrogen carbonate and the mixture was concentrated. The residue was extracted with ethyl acetate, dried (MgSO₄) and concentrated. The residue was subjected to aminopropyl silica gel column chromatography (developing solvent: hexane/ethyl acetate=1/1) to give the title compound (110 mg, yield 46%) as a colorless amorphous powder.

¹H NMR (400 MHz, CDCl₃) δ ppm: 0.70-1.77 (m, 9 H), 1.33 (d, J=6.8 Hz, 3 H), 2.14-2.32 (m, 3 H), 2.27 (s, 6 H), 3.39-3.48 (m, 2 H), 3.65 (d, J=3.7 Hz, 1 H), 3.95-4.01 (m, 1 H), 7.07-7.30 (m, 9 H), 7.33 (d, J=7.6 Hz, 1 H), 7.38 (d, J=8.1 Hz, 1 H), 7.50 (s, 1 H), 7.68 (d, J=8.1 Hz, 1 H), 7.87 (d, J=7.6 Hz, 1 H), 8.09 (s, 1 H), 9.59 (s, 1 H).

LC/MS (ESI) m/z 523 (M+H⁺).

The compound described in the following Example 124 was produced in the similar manner as in Example 123.

### Example 124

### (2R,3S)-N-{3-[(dimethylamino)methyl]phenyl}-3-(1H-indol-3-yl)-2-{[(1-phenylpiperidin-4-yl)methyl]amino}butanamide

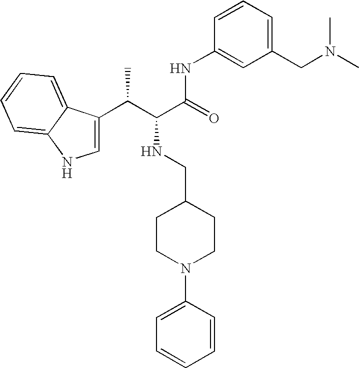

¹H NMR (400 MHz, CDCl₃) δ ppm: 0.70-1.45 (m, 4 H), 1.33 (d, J=7.1 Hz, 3 H), 2.09 (dt, J=12.0, 2.6 Hz, 1 H), 2.19-2.34 (m, 4 H), 2.27 (s, 6 H), 2.45 (dt, J=12.0, 2.6 Hz, 1 H), 3.33-3.50 (m, 4 H), 3.67 (d, J=3.7 Hz, 1 H), 3.95-4.02 (m, 1 H), 6.78-6.83 (m, 3 H), 7.05-7.09 (m, 2 H), 7.14-7.23 (m, 4 H), 7.31 (t, J=7.8 Hz, 1 H), 7.39 (d, J=8.1 Hz, 1 H), 7.48 (s, 1 H), 7.67 (dd, J=8.1, 2.0 Hz, 1 H), 7.85 (d, J=7.8 Hz, 1 H), 8.09 (s, 1 H), 9.51 (s, 1 H).

LC/MS (ESI) m/z 524 (M+H⁺).

The compounds described in the following Examples 125-126 were produced in the similar manner as in Example 1.

### Example 125

### tert-butyl 4-({[(1R,2S)-1-[({5-[(dimethylamino)methyl]-2-ethoxyphenyl}amino)carbonyl]-2-(1H-indol-3-yl)propyl]amino)carbonyl)piperazine-1-carboxylate

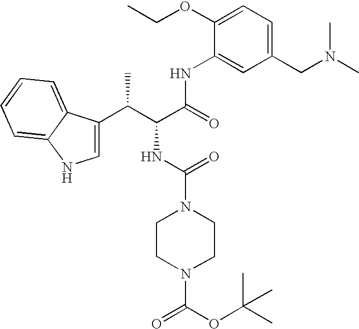

¹H NMR (400 MHz, CDCl₃) δ ppm: 1.23 (t, J=7.0 Hz, 3 H), 1.47 (s, 9 H), 1.56 (d, J=7.3 Hz, 3 H), 2.20 (s, 6 H), 3.21-3.47 (m, 11 H), 3.56-3.67 (m, 1 H), 3.79-3.96 (m, 1 H), 4.86 (dd, J=7.5 Hz, 1 H), 5.29 (d, J=7.8 Hz, 1 H), 6.69 (d, J=8.3 Hz, 1 H), 6.93 (dd, J=8.3, 2.0 Hz, 1 H), 7.04-7.35 (m, 4 H), 7.72 (d, J=7.8 Hz, 1 H), 7.86 (s, 1 H), 8.04 (s, 1 H), 8.17 (d, J=2.2 Hz, 1 H).

### Example 126

### tert-butyl 4-({[(1R,2S)-1-[({5-[(dimethylamino)methyl]-2-methoxyphenyl)amino)carbonyl]-2-(1H-indol-3-yl)propyl]amino)carbonyl)piperazine-1-carboxylate

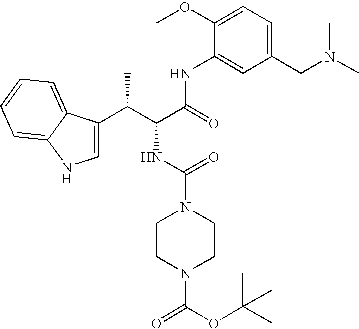

¹H NMR (400 MHz, CDCl₃) δ ppm: 1.47 (s, 9 H), 1.55 (d, J=7.3 Hz, 3 H), 2.21 (s, 6 H), 3.29-3.57 (m, 11 H), 3.54 (s, 3 H), 4.90 (t, J=8.1 Hz, 1 H), 5.38 (d, J=8.1 Hz, 1 H), 6.65 (d, J=8.3 Hz, 1 H), 6.92 (dd, J=8.3, 2.0 Hz, 1 H), 7.02-7.19 (m, 3 H), 7.32 (d, J=8.1 Hz, 1 H), 7.70 (s, 1 H), 7.76 (d, J=7.8 Hz, 1 H), 8.10 (d, J=2.2 Hz, 1 H), 8.20 (s, 1 H).

### Example 127

### N-[(1R,2S)-1-[({5-[(dimethylamino)methyl]-2-ethoxyphenyl}amino)carbonyl]-2-(1H-indol-3-yl)propyl]piperazine-1-carboxamide

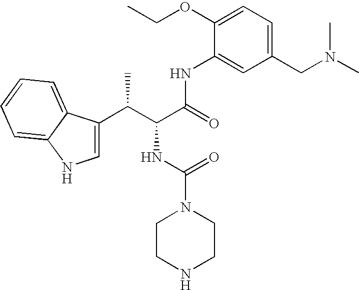

tert-Butyl 4-({[(1R,2S)-1-[({5-[(dimethylamino)methyl]-2-ethoxyphenyl}amino)carbonyl]-2-(1H-indol-3-yl)propyl]amino}carbonyl)piperazine-1-carboxylate (1.1 g) was mixed with trifluoroacetic acid (15 mL) and the mixture was stirred at room temperature for 12 hrs. The reaction solution was concentrated and the residue was mixed with saturated aqueous solution of sodium hydrogen carbonate. The mixture was extracted with ethyl acetate. The extract was dried (MgSO₄) and concentrated under reduced pressure. The residue was subjected to silica gel column chromatography to give the title compound (250 mg, yield 27%) as colorless crystals.

¹H NMR (400 MHz, CDCl₃) δ ppm: 1.23 (t, J=7.0 Hz, 3 H), 1.57 (d, J=7.3 Hz, 3 H), 2.30 (s, 6 H), 2.77-2.88 (m, 4 H), 3.22-3.40 (m, 4 H), 3.45 (s, 2 H), 3.56-3.67 (m, 1 H), 3.78-3.95 (m, 2 H), 4.86 (t, J=7.5 Hz, 1 H), 5.26 (d, J=7.8 Hz, 1 H), 6.70 (d, J=8.3 Hz, 1 H), 6.96 (dd, J=8.4, 2.1 Hz, 1 H), 7.03-7.11 (m, 4 H), 7.15 (t, J=7.6 Hz, 1 H), 7.32 (d, J=8.1 Hz, 1 H), 7.73 (d, J=8.1 Hz, 1 H), 7.91 (s, 1 H), 8.08 (s, 1 H), 8.19 (d, J=2.0 Hz, 1 H).

LC/MS (ESI) m/z 507 (M+H⁺).

### Example 128

### N-[(1R,2S)-1-[({5-[(dimethylamino)methyl]-2-methoxyphenyl}amino)carbonyl]-2-(1H-indol-3-yl)propyl]piperazine-1-carboxamide dihydrochloride

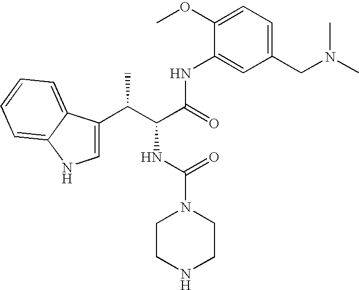

tert-Butyl 4-({[(1R,2S)-1-[({5-[(dimethylamino)methyl]-2-methoxyphenyl}amino)carbonyl]-2-(1H-indol-3-yl)propyl]amino}carbonyl)piperazine-1-carboxylate (710 mg) was dissolved in ethyl acetate (6 mL) and 4N hydrochloric acid-ethyl acetate solution was added at room temperature. The mixture was stirred for 30 min. The resulting precipitates were collected by filtration, dried under reduced pressure to give the title compound (680 mg, yield 100%) as colorless crystals.

LC/MS (ESI) m/z 493 (M+H⁺)-2HCl.

### Example 129

### N-[(1R,2S)-1-[({5-[(dimethylamino)methyl]-2-ethoxyphenyl}amino)carbonyl]-2-(1H-indol-3-yl)propyl]-4-(4-fluorobenzoyl)piperazine-1-carboxamide

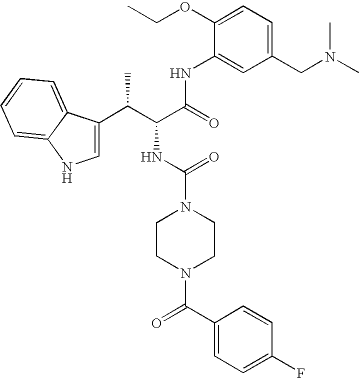

N-[(1R,2S)-1-[({5-[(Dimethylamino)methyl]-2-ethoxyphenyl}amino)carbonyl]-2-(1H-indol-3-yl)propyl]piperazine-1-carboxamide (80 mg, 0.157 mmol) and 4-fluorobenzoic acid (26 mg, 0.188 mmol) were dissolved in a mixed solution of THF (0.5 mL)-acetonitrile (0.5 mL), and WSC (41 mg, 0.21 mmol) and HOBt (34 mg, 0.22 mmol) were added. The mixture was stirred at room temperature for 12 hrs. To the reaction solution was added saturated aqueous solution of sodium hydrogen carbonate and the mixture was extracted with ethyl acetate. The extract was dried (MgSO₄) and concentrated under reduced pressure. The residue was purified (ethyl acetate/ethanol=20/1) using Megabond Elute (product name)(NH2) to give the title compound (80 mg, yield 81%) as an amorphous powder.

¹H NMR (400 MHz, CDCl₃) δ ppm: 1.23 (t, J=6.4 Hz, 3 H), 1.56 (d, J=6.6 Hz, 3 H), 2.20 (s, 6 H), 3.32-3.65 (m, 12 H), 3.84-3.91 (m, 2 H), 4.85 (t, J=7.5 Hz, 1 H), 5.43 (d, J=6.8 Hz, 1 H), 6.69 (d, J=7.8 Hz, 1 H), 6.92 (d, J=7.6 Hz, 1 H), 7.02-7.40, (m, 8 H), 7.70 (d, J=7.1 Hz, 1 H), 7.85 (s, 1 H), 8.17 (s, 1 H), 8.31 (s, 1 H).

LC/MS (ESI) m/z 629 (M+H⁺).

The compounds described in the following Examples 130-131 were produced in the similar manner as in Example 129.

### Example 130

### N-[(1R,2S)-1-[({5-[(dimethylamino)methyl]-2-ethoxyphenyl}amino)carbonyl]-2-(1H-indol-3-yl)propyl]-4-(pyridin-3-ylcarbonyl)piperazine-1-carboxamide

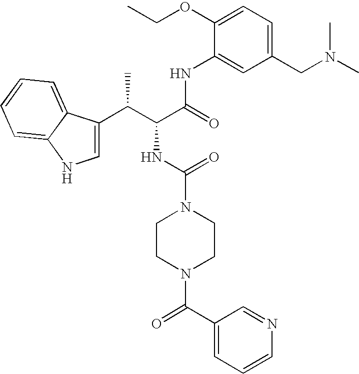

¹H NMR (400 MHz, CDCl₃) δ ppm: 1.23 (t, J=7.0 Hz, 3 H), 1.56 (d, J=7.1 Hz, 3 H), 2.20 (s, 6 H), 3.32-3.93 (m, 13 H), 4.85 (t, J=7.5 Hz, 1 H), 5.43 (d, J=7.5 Hz, 1 H), 6.69 (d, J=8.3 Hz, 1 H), 6.92-7.40 (m, 6 H), 6.68-7.76 (m, 2 H), 7.87 (s, 1 H), 8.16 (s, 1 H), 8.31 (s, 1 H), 8.65-8.69 (m, 2H).

LC/MS (ESI) m/z 612 (M+H⁺).

### Example 131

### N-[(1R,2S)-1-[({5-[(dimethylamino)methyl]-2-ethoxyphenyl}amino)carbonyl]-2-(1H-indol-3-yl)propyl]-4-(2-furoyl)piperazine-1-carboxamide

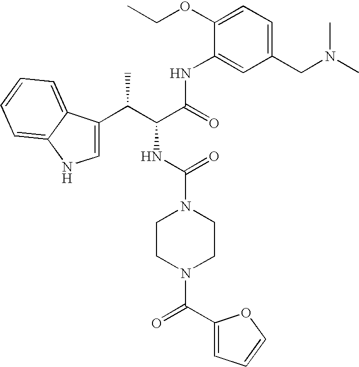

¹H NMR (400 MHz, CDCl₃) δ ppm: 1.23 (t, J=7.0 Hz, 3 H), 1.57 (d, J=7.3 Hz, 3 H), 2.21 (s, 6 H), 3.26-3.97 (m, 13 H), 4.87 (t, J=7.5 Hz, 1 H), 5.39 (d, J=7.6 Hz, 1 H), 6.50 (dd, J=3.4, 1.7 Hz, 1 H), 6.70 (d, J=8.3 Hz, 1 H), 6.93 (dd, J=8.3, 2.0 Hz, 1 H), 6.99-7.21 (m, 6 H), 7.31 (d, J=8.1 Hz, 1 H), 7.50 (s, 1 H), 7.72 (d, J=7.8 Hz, 1 H), 7.88 (s, 1 H)

LC/MS (ESI) m/z 601 (M+H⁺).

### Example 132

### N-[(1R,2S)-1-[({5-[(dimethylamino)methyl]-2-methoxyphenyl}amino)carbonyl]-2-(1H-indol-3-yl)propyl]-4-(4-fluorobenzoyl)piperazine-1-carboxamide

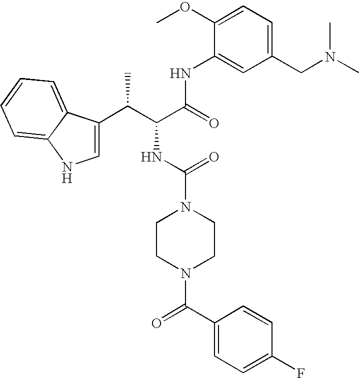

N-[(1R,2S)-1-[({5-[(Dimethylamino)methyl]-2-methoxyphenyl}amino)carbonyl]-2-(1H-indol-3-yl)propyl]piperazine-1-carboxamide dihydrochloride (120 mg, 0.26 mmol), 4-fluorobenzoic acid (40 mg, 0.28 mmol) and triethylamine (0.1 mL) were dissolved in a mixed solution of THF (1.0 mL)-acetonitrile (1.0 mL), and WSC (67 mg, 0.35 mmol) and HOBt (55 mg, 0.36 mmol) were added. The mixture was stirred at room temperature for 12 hrs. To the reaction solution was added saturated aqueous solution of sodium hydrogen carbonate and the mixture was extracted with ethyl acetate. The extract was dried (MgSO₄) and concentrated under reduced pressure. The residue was purified (developing solvent: ethyl acetate/ethanol=20/1) using Megabond Elute (product name)(NH2) to give the title compound (55 mg, yield 35%) as an amorphous powder.

¹H NMR (400 MHz, CDCl₃) δ ppm: 1.56 (d, J=7.1 Hz, 3 H), 2.20 (S, 6 H), 3.22-3.88 (m, 14 H), 4.90 (t, J=8.1 Hz, 2 H), 5.43 (s, 2 H), 6.66 (d, J=8.1 Hz, 1 H), 6.93 (d, J=8.3 Hz, 1 H), 7.03-7.36 (m, 5 H), 7.42 (dd, J=8.1, 5.6 Hz, 2 H), 7.65 (s, 1 H), 7.76 (d, J=7.8 Hz, 1 H), 8.05-8.17 (m, J=2.0 Hz, 2 H).

LC/MS (ESI) m/z 615 (M+H⁺).

The compounds described in the following Examples 133-134 were produced in the similar manner as in Example 132.

### Example 133

### N-[(1R,2S)-1-[({5-[(dimethylamino)methyl]-2-methoxyphenyl}amino)carbonyl]-2-(1H-indol-3-yl)propyl]-4-(pyridin-3-ylcarbonyl)piperazine-1-carboxamide

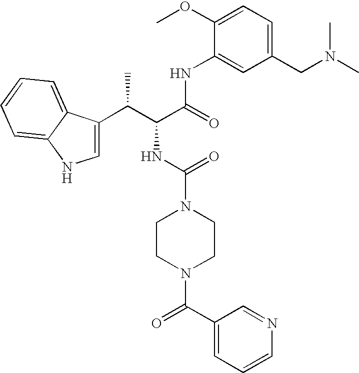

¹H NMR (400 MHz, CDCl₃) δ ppm: 1.56 (d, J=7.3 Hz, 3 H), 2.20 (s, 6 H), 3.24-3.93 (m, 14 H), 4.90 (t, J=8.1 Hz, 1 H), 5.43 (d, J=8.1 Hz, 1 H), 6.66 (d, J=8.3 Hz, 1 H), 6.93 (dd, J=8.4, 1.8 Hz, 1 H), 7.03-7.36 (m, 5 H), 7.39 (dd, J=7.6, 4.6 Hz, 1 H), 7.65 (s, 1 H), 7.72-7.79 (m, 2 H), 8.11 (d, J=2.0 Hz, 1 H), 8.61-8.74 (m, 2 H).

LC/MS (ESI) m/z 598 (M+H⁺).

### Example 134

### N-[(1R,2S)-1-[({5-[(dimethylamino)methyl]-2-methoxyphenyl}amino)carbonyl]-2-(1H-indol-3-yl)propyl]-4-(2-furoyl)piperazine-1-carboxamide

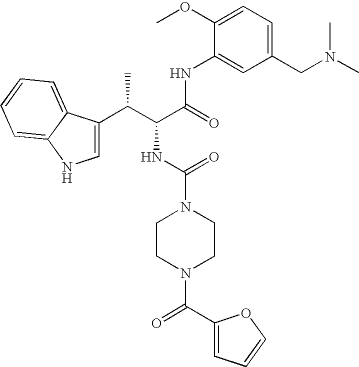

¹H NMR (400 MHz, CDCl₃) δ ppm: 1.57 (d, J=7.1 Hz, 3 H), 2.19-2.20 (m, 6 H), 3.31 (s, 2 H), 3.40-3.58 (m, 9 H), 3.81 (s, 3 H), 4.92 (t, J=8.1 Hz, 1 H), 5.39 (d, J=7.8 Hz, 1 H), 6.50 (dd, J=3.4, 1.7 Hz, 1 H), 6.66 (d, J=8.3 Hz, 1 H), 6.93 (dd, J=8.3, 2.0 Hz, 1 H), 7.02-7.31 (m, 5 H), 7.34 (d, J=8.1 Hz, 1 H), 7.51 (d, J=1.2 Hz, 1 H), 7.67 (s, 1 H), 7.77 (d, J=7.8 Hz, 1 H), 8.05 (s, 1 H), 8.12 (d, J=2.2 Hz, 1 H).

LC/MS (ESI) m/z 587 (M+H⁺).

### Example 135

### N-[(1R,2S)-1-[({5-[(dimethylamino)methyl]-2-ethoxyphenyl}amino)carbonyl]-2-(1H-indol-3-yl)propyl]-3-oxo-4-phenylpiperazine-1-carboxamide

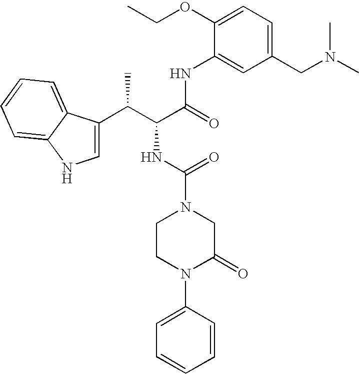

5-[(Dimethylamino)methyl]-2-ethoxyaniline dihydrochloride (58 mg, 0.216 mmol) was dissolved in a mixed solution of THF (1.0 mL)-acetonitrile (1.0 mL), and triethylamine (0.07 mL) was added. The mixture was stirred at room temperature for 30 min. To the reaction solution were added (2R,3S)-2-({[(2-anilinoethyl)(carboxymethyl)amino]carbonyl}amino)-3-(1H-indol-3-yl)butanoic acid (100 mg, 0.238 mmol), WSC (52 mg, 0.27 mmol) and HOBt (43 mg, 0.28 mmol) and the mixture was stirred at room temperature for 12 hrs. To the reaction solution was added saturated aqueous solution of sodium hydrogen carbonate and the mixture was extracted with ethyl acetate. The extract was dried (MgSO₄) and concentrated under reduced pressure. The residue was purified (developing solvent: chloroform/ethanol=9/1) using Megabond Elute (product name)(NH2) to give the title compound (10 mg, yield 7%) as colorless crystals.

¹H NMR (400 MHz, CDCl₃) δ ppm: 1.25 (t, J=7.0 Hz, 3 H), 1.57 (d, J=7.1 Hz, 3 H), 2.21 (s, 6 H), 3.33 (s, 2 H), 3.60-3.99 (m, 7 H), 4.10 (d, J=12.9 Hz, 1 H), 4.21 (d, J=12.9 Hz, 1 H), 4.86 (t, J=7.5 Hz, 1 H), 5.35 (d, J=7.6 Hz, 1 H), 6.70 (d, J=8.3 Hz, 1 H), 6.94 (dd, J=8.3, 2.0 Hz, 1 H), 7.00-7.21 (m, 3 H), 7.23-7.46 (m, 6 H), 7.73 (d, J=7.8 Hz, 1 H), 7.92 (s, 1 H), 8.13-8.27 (m, 2 H).

LC/MS (ESI) m/z 597 (M+H⁺).

### Example 136

### 4-(4-chlorophenyl)-N-[(1R,2S)-1-[({3-[(dimethylamino)methyl]phenyl}amino)carbonyl]-2-(1H-indol-3-yl)propyl]piperazine-1-carboxamide

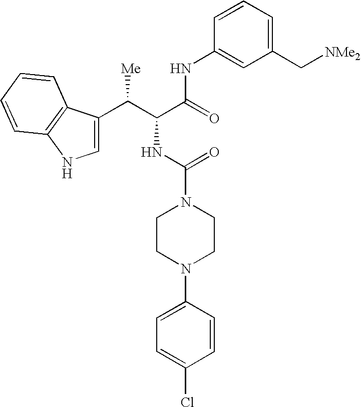

A mixture of (2R,3S)-2-({[4-(4-chlorophenyl)piperazin-1-yl]carbonyl}amino)-3-(1H-indol-3-yl)butanoic acid (220 mg, 0.500 mmol), 3-dimethylaminomethylaniline (90.1 mg, 0.600 mmol), WSC (115 mg, 0.600 mmol), HOBt (91.9 mg, 0.600 mmol), THF (1 mL) and acetonitrile (1 mL) was stirred at room temperature for 2 hrs. To the reaction mixture were added saturated aqueous solution of sodium hydrogen carbonate (5 mL) and water (10 mL) and the mixture was further stirred for 10 min. The crystals were collected by filtration, dried and dissolved in THF. The resulting solution was passed through an aminopropyl silica gel layer and concentrated under reduced pressure. The residue was recrystallized from THF to give the title compound as a colorless crystalline powder (229 mg, yield 80%).

¹H NMR (300 MHz, DMSO-d₆) δ ppm: 1.32 (d, J=7.2 Hz, 3 H), 2.11 (s, 6 H), 3.02-3.14 (m, 4 H), 3.28 (s, 2 H), 3.42-3.66 (m, 5 H), 4.64 (t, J=8.7 Hz, 1 H), 6.65 (d, J=8.7 Hz, 1 H), 6.88 (d, J=7.5 Hz, 1 H), 6.91-7.04 (m, 4 H), 7.13 (t, J=7.8 Hz, 1 H), 7.22-7.29 (m, 4 H), 7.33-7.39 (m, 2 H), 7.60 (d, J=7.5 Hz, 1 H), 9.81 (s, 1 H), 10.80 (d, J=1.9 Hz, 1 H).

LC/MS (ESI) m/z 573 (M+H⁺).

The compounds described in the following Examples 137-158 were produced in the similar manner as in Example 136.

### Example 137

### 4-(2-chlorophenyl)-N-[(1R,2S)-1-[({3-[(dimethylamino)methyl]phenyl}amino)carbonyl]-2-(1H-indol-3-yl)propyl]piperazine-1-carboxamide

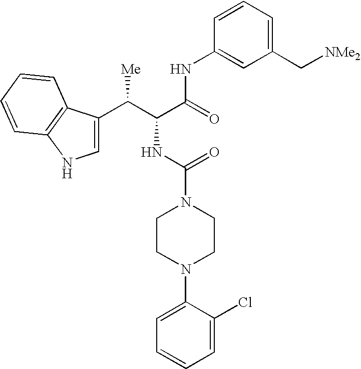

¹H NMR (300 MHz, DMSO-d₆) δ ppm: 1.33 (d, J=7.0 Hz, 3 H), 2.10 (s, 6 H), 2.84-2.96 (m, 4 H), 3.27 (s, 2 H), 3.43-3.56 (m, 4 H), 3.58-3.67 (m, 1 H), 4.65 (t, J=8.6 Hz, 1 H), 6.59 (d, J=8.6 Hz, 1 H), 6.89 (d, J=7.4 Hz, 1 H), 6.93-6.98 (m, 1 H), 7.00-7.16 (m, 4 H), 7.25-7.44 (m, 6 H), 7.61 (d, J=7.7 Hz, 1 H), 9.81 (s, 1 H), 10.81 (d, J=1.3 Hz, 1 H).

LC/MS (ESI) m/z 573 (M+H⁺).

### Example 138

### 4-(2-chlorophenyl)-N-[(1R,2S)-1-[({5-[(dimethylamino)methyl]-2-ethoxyphenyl}amino)carbonyl]-2-(1H-indol-3-yl)propyl]piperazine-1-carboxamide

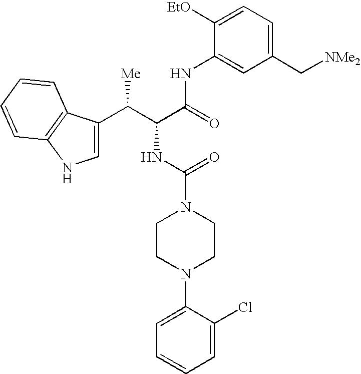

¹H NMR (300 MHz, CDCl₃) δ ppm: 1.23 (t, J=7.0 Hz, 3 H), 1.58 (d, J=7.2 Hz, 3 H), 2.21 (s, 6 H), 2.93-3.05 (m, 4 H), 3.32 (s, 2 H), 3.45-3.69 (m, 5 H), 3.80-3.96 (m, 2 H), 4.90 (t, J=7.4 Hz, 1 H), 5.36 (d, J=7.7 Hz, 1 H), 6.69 (d, J=8.3 Hz, 1 H), 6.93 (dd, J=2.1, 8.3 Hz, 1 H), 6.97-7.03 (m, 2 H), 7.06-7.09 (m, 2 H), 7.13-7.18 (m, 1 H), 7.20-7.26 (m, 1 H), 7.32 (d, J=7.5 Hz, 1 H), 7.37 (dd, J=1.6, 8.2 Hz, 1 H), 7.75 (d, J=7.7 Hz, 1 H), 7.91 (s, 1 H), 8.06 (brs, 1H), 8.18 (d, J=1.9 Hz, 1 H).

LC/MS (ESI) m/z 617 (M+H⁺).

### Example 139

### N-[(1R,2S)-1-[({3-[(dimethylamino)methyl]phenyl}amino)carbonyl]-2-(1H-indol-3-yl)propyl]-4-(4-methylphenyl)piperazine-1-carboxamide

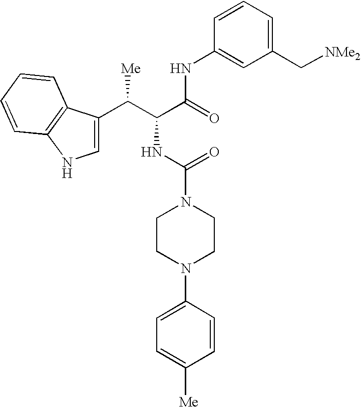

¹H NMR (300 MHz, DMSO-d₆) δ ppm: 1.32 (d, J=7.2 Hz, 3 H), 2.10 (s, 6 H), 2.20 (s, 3 H), 2.95-3.07 (m, 4 H), 3.26 (s, 2 H), 3.41-3.66 (m, 5 H), 4.64 (t, J=8.6 Hz, 1 H), 6.63 (d, J=8.6 Hz, 1 H), 6.84-7.06 (m, 7 H), 7.13 (t, J=7.7 Hz, 1 H), 7.26 (d, J=7.9 Hz, 1 H), 7.29 (d, J=2.3 Hz, 1 H), 7.33-7.38 (m, 2 H), 7.60 (d, J=7.7 Hz, 1 H), 9.81 (s, 1 H), 10.79 (d, J=2.3 Hz, 1 H).

LC/MS (ESI) m/z 553 (M+H⁺).

### Example 140

### N-[(1R,2S)-1-[({3-[(dimethylamino)methyl]phenyl}amino)carbonyl]-2-(1H-indol-3-yl)propyl]-4-[4-(trifluoromethyl)phenyl]piperazine-1-carboxamide

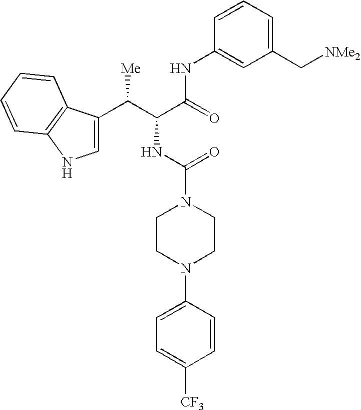

¹H NMR (300 MHz, DMSO-d₆) δ ppm: 1.32 (d, J=7.0 Hz, 3 H), 2.11 (s, 6 H), 3.19-3.31 (m, 6 H), 3.44-3.67 (m, 5 H), 4.65 (t, J=8.7 Hz, 1 H), 6.67 (d, J=8.7 Hz, 1 H), 6.87-7.04 (m, 3 H), 7.06-7.16 (m, 3 H), 7.26 (d, J=7.9 Hz, 1 H), 7.29 (d, J=2.3 Hz, 1 H), 7.34-7.38 (m, 2 H), 7.49-7.53 (m, 2 H), 7.60 (d, J=7.7 Hz, 1 H), 9.82 (s, 1 H), 10.79 (d, J=2.3 Hz, 1 H).

LC/MS (ESI) m/z 607 (M+H⁺).

### Example 141

### N-[(1R,2S)-1-[({3-[(dimethylamino)methyl]phenyl}amino)carbonyl]-2-(1H-indol-3-yl)propyl]-4-(4-methoxyphenyl)piperazine-1-carboxamide

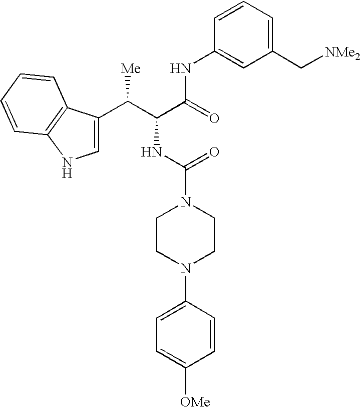

¹H NMR (300 MHz, DMSO-d₆) δ ppm: 1.32 (d, J=7.2 Hz, 1 H), 2.10 (s, 6 H), 2.89-3.00 (m, 4 H), 3.26 (s, 2 H), 3.41-3.66 (m, 5 H), 3.69 (s, 3 H), 4.64 (t, J=8.7 Hz, 1 H), 6.62 (d, J=8.7 Hz, 1 H), 6.80-6.85 (m, 2 H), 6.87-6.97 (m, 4 H), 6.99-7.04 (m, 1 H), 7.13 (t, J=7.8 Hz, 1 H), 7.26 (d, J=7.9 Hz, 1 H), 7.29 (d, J=2.3 Hz, 1 H), 7.33-7.38 (m, 2 H), 7.60 (d, J=7.7 Hz, 1 H), 9.81 (s, 1 H), 10.80 (d, J=2.3 Hz, 1 H).

LC/MS (ESI) m/z 569 (M+H⁺).

### Example 142

### N-[(1R,2S)-1-[({3-[(dimethylamino)methyl]phenyl}amino)carbonyl]-2-(1H-indol-3-yl)propyl]-4-(2-fluorophenyl)piperazine-1-carboxamide

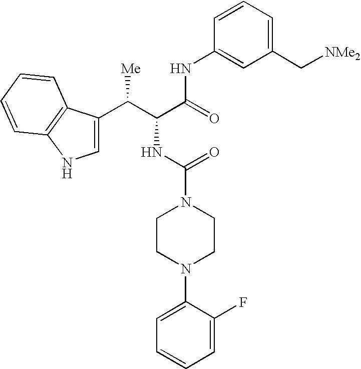

¹H NMR (300 MHz, DMSO-d₆) δ ppm: 1.32 (d, J=7.0 Hz, 3 H), 2.10 (s, 6 H), 2.88-3.00 (m, 4 H), 3.27 (s, 2 H), 3.43-3.67 (m, 5 H), 4.65 (t, J=8.5 Hz, 1 H), 6.61 (d, J=8.5 Hz, 1 H), 6.87-7.18 (m, 8 H), 7.26 (d, J=7.9 Hz, 1 H), 7.30 (d, J=2.3 Hz, 1 H), 7.35-7.38 (m, 2 H), 7.61 (d, J=7.7 Hz, 1 H), 9.81 (s, 1 H), 10.80 (d, J=2.3 Hz, 1 H).

LC/MS (ESI) m/z 557 (M+H⁺).

### Example 143

### N-[(1R,2S)-1-[({3-[(dimethylamino)methyl]phenyl}amino)carbonyl]-2-(1H-indol-3-yl)propyl]-4-(4-fluoro-2-methylphenyl)piperazine-1-carboxamide

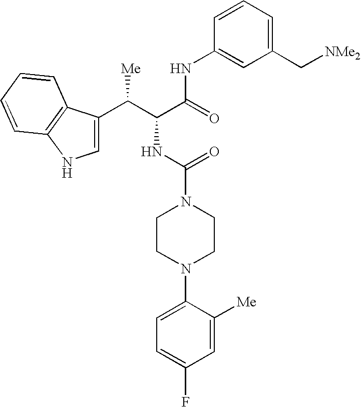

¹H NMR (300 MHz, CDCl₃) δ ppm: 1.58 (d, J=7.0 Hz, 3 H), 2.19 (s, 6 H), 2.29 (s, 3 H), 2.72-2.85 (m, 4 H), 3.24-3.33 (m, 2 H), 3.43-3.62 (m, 5 H), 4.83 (t, J=8.5 Hz, 1 H), 5.53 (d, J=8.1 Hz, 1 H), 6.81-6.98 (m, 6 H), 7.08-7.15 (m, 3 H), 7.16-7.21 (m, 1 H), 7.35 (d, J=7.9 Hz, 1 H), 7.56 (brs, 1 H), 7.78 (d, J=7.5 Hz, 1 H), 8.39 (brs, 1 H).

LC/MS (ESI) m/z 571 (M+H⁺).

### Example 144

### 4-(3-chlorophenyl)-N-[(1R,2S)-1-[({3-[(dimethylamino)methyl]phenyl}amino)carbonyl]-2-(1H-indol-3-yl)propyl]piperazine-1-carboxamide

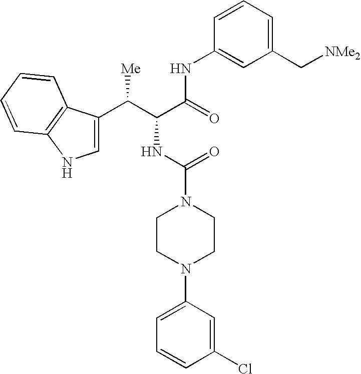

¹H NMR (300 MHz, DMSO-d₆) δ ppm: 1.32 (d, J=7.2 Hz, 3 H), 2.10 (s, 6 H), 3.07-3.19 (m, 4 H), 3.26 (s, 2 H), 3.41-3.56 (m, 4 H), 3.56-3.67 (m, 1 H), 4.64 (t, J=8.6 Hz, 1 H), 6.66 (d, J=8.6 Hz, 1 H), 6.80 (dd, J=1.2, 7.8 Hz, 1 H), 6.87-7.04 (m, 5 H), 7.13 (t, J=7.8 Hz, 1 H), 7.19-7.29 (m, 3 H), 7.33-7.38 (m, 2 H), 7.60 (d, J=7.7 Hz, 1 H), 9.81 (s, 1 H), 10.80 (d, J=1.7 Hz, 1 H).

LC/MS (ESI) m/z 573 (M+H⁺).

### Example 145

### 4-(3-chlorophenyl)-N-[(1R,2S)-1-[({5-[(dimethylamino)methyl]-2-ethoxyphenyl}amino)carbonyl]-2-(1H-indol-3-yl)propyl]piperazine-1-carboxamide

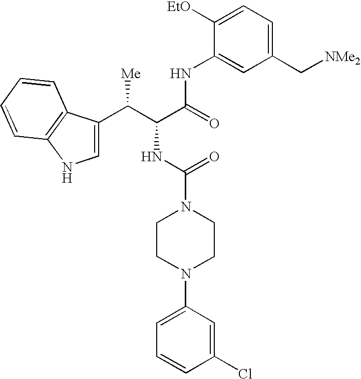

¹H NMR (300 MHz, CDCl₃) δ ppm: 1.23 (t, J=7.0 Hz, 3 H), 1.58 (d, J=7.4 Hz, 3 H), 2.20 (s, 6 H), 3.08-3.20 (m, 4 H), 3.32 (s, 2 H), 3.41-3.68 (m, 5 H), 3.79-3.96 (m, 2 H), 4.88 (t, J=7.5 Hz, 1 H), 5.37 (d, J=7.9 Hz, 1 H), 6.69 (d, J=8.4 Hz, 1 H), 6.76 (ddd, J=0.9, 2.2, 8.4 Hz, 1 H), 6.82-6.86 (m, 2 H), 6.93 (dd, J=2.2, 8.4 Hz, 1 H), 7.05-7.21 (m, 4 H), 7.32 (d, J=7.9 Hz, 1 H), 7.73 (d, J=7.9 Hz, 1 H), 7.87 (s, 1 H), 8.06 (brs, 1 H), 8.17 (d, J=2.1 Hz, 1 H).

LC/MS (ESI) m/z 617 (M+H⁺).

### Example 146

### N-[(1R,2S)-1-[({3-[(dimethylamino)methyl]phenyl}amino)carbonyl]-2-(1H-indol-3-yl)propyl]-4-(3-fluorophenyl)piperazine-1-carboxamide

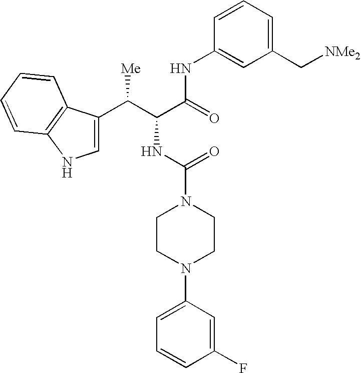

¹H NMR (300 MHz, DMSO-d₆) δ ppm: 1.32 (d, J=7.2 Hz, 1 H), 2.15 (s, 6 H), 3.08-3.20 (m, 4 H), 3.32 (s, 2 H), 3.42-3.67 (m, 5 H), 4.64 (t, J=8.6 Hz, 1 H), 6.53-6.59 (m, 1 H), 6.67 (d, J=8.9 Hz, 1 H), 6.74-6.80 (m, 2 H), 6.89-6.97 (m, 2 H), 6.98-7.04 (m, 1 H), 7.12-7.29 (m, 4 H), 7.35 (d, J=8.3 Hz, 1 H), 7.40 (s, 1 H), 7.60 (d, J=7.5 Hz, 1 H), 9.83 (s, 1 H), 10.80 (d, J=1.9 Hz, 1 H).

LC/MS (ESI) m/z 557 (M+H⁺).

### Example 147

### N-[(1R,2S)-1-[({3-[(dimethylamino)methyl]phenyl}amino)carbonyl]-2-(1H-indol-3-yl)propyl]-4-(4-fluoro-2-formylphenyl)piperazine-1-carboxamide

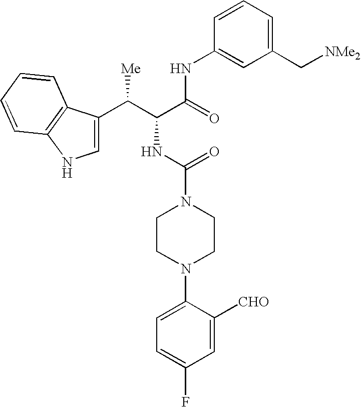

¹H NMR (300 MHz, DMSO-d₆) δ ppm: 1.33 (d, J=7.0 Hz, 3 H), 2.15 (s, 6 H), 2.90-2.98 (m, 4 H), 3.33 (s, 2 H), 3.48-3.67 (m, 5 H), 4.65 (t, J=8.7 Hz, 1 H), 6.65 (d, J=8.5 Hz, 1 H), 6.89-7.04 (m, 3 H), 7.15 (t, J=7.7 Hz, 1 H), 7.25-7.53 (m, 7 H), 7.61 (d, J=7.5 Hz, 1 H), 9.83 (s, 1 H), 10.27 (d, J=3.0 Hz, 1 H), 10.80 (d, J=2.1 Hz, 1 H).

LC/MS (ESI) m/z 585 (M+H⁺).

### Example 148

### 4-(cyclopropylmethyl)-N-[(1R,2S)-1-[({3-[(dimethylamino)methyl]phenyl}amino)carbonyl]-2-(1H-indol-3-yl)propyl]piperazine-1-carboxamide

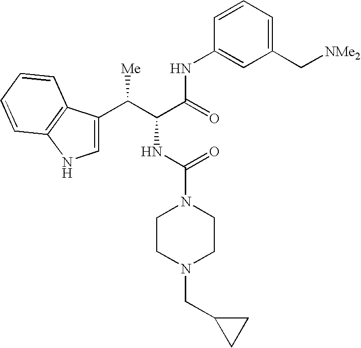

¹H NMR (300 MHz, DMSO-d₆) δ ppm: 0.04-0.09 (m, 2 H), 0.43-0.48 (m, 2 H), 0.75-0.88 (m, 1 H), 1.30 (d, J=7.0 Hz, 3 H), 2.10 (s, 6 H), 2.16 (d, J=6.4 Hz, 2 H), 2.30-2.42 (m, 4 H), 3.27-3.40 (m, 6 H), 3.54-3.64 (m, 1 H), 4.61 (t, J=8.5 Hz, 1 H), 6.44 (d, J=8.7 Hz, 1 H), 6.88 (d, J=7.5 Hz, 1 H), 6.92-6.96 (m, 1 H), 6.99-7.04 (m, 1 H), 7.13 (t, J=7.6 Hz, 1 H), 7.25-7.28 (m, 2 H), 7.34-7.37 (m, 2 H), 7.59 (d, J=7.7 Hz, 1 H), 9.78 (s, 1 H), 10.79 (d, J=1.7 Hz, 1 H).

LC/MS (ESI) m/z 517 (M+H⁺).

### Example 149

### 4-(cyclopropylmethyl)-N-[(1R,2S)-1-[({5-[(dimethylamino)methyl]-2-ethoxyphenyl}amino)carbonyl]-2-(1H-indol-3-yl)propyl]piperazine-1-carboxamide

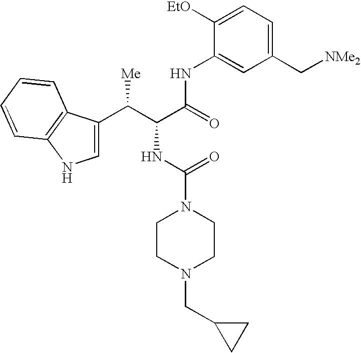

¹H NMR (300 MHz, CDCl₃) δ ppm: 0.08-0.13 (m, 2 H), 0.50-0.56 (m, 2 H), 0.78-0.92 (m, 1 H), 1.12 (t, J=7.2 Hz, 3 H), 1.56 (d, J=7.4 Hz, 3 H), 2.20 (s, 6 H), 2.25 (d, J=6.6 Hz, 2 H), 2.41-2.54 (m, 4 H), 3.32-3.47 (m, 6 H), 3.57-3.66 (m, 1 H), 3.78-3.95 (m, 2 H), 4.87 (t, J=7.4 Hz, 1 H), 5.29 (d, J=7.7 Hz, 1 H), 6.68 (d, J=8.3 Hz, 1 H), 6.92 (dd, J=1.9, 8.3 Hz, 1 H), 7.05-7.17 (m, 3 H), 7.31 (d, J=8.1 Hz, 1 H), 7.73 (d, J=7.7 Hz, 1 H), 7.91 (s, 1 H), 8.07 (s, 1 H), 8.17 (d, J=1.9 Hz, 1 H).

LC/MS (ESI) m/z 561 (M+H⁺).

### Example 150

### N-[(1R,2S)-1-[({3-[(dimethylamino)methyl]phenyl}amino)carbonyl]-2-(1H-indol-3-yl)propyl]-N′-phenylpiperazine-1,4-dicarboxamide

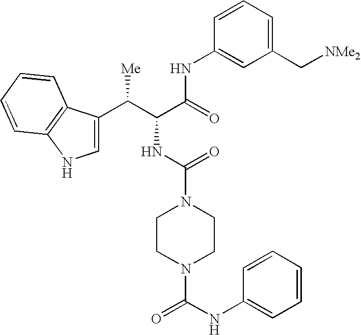

¹H NMR (300 MHz, DMSO-d₆) δ ppm: 1.32 (t, J=7.2 Hz, 3 H), 2.12 (s, 6 H), 3.29 (s, 2 H), 3.33-3.49 (m, 8 H), 3.56-3.66 (m, 1 H), 4.64 (t, J=8.7 Hz, 1 H), 6.62 (d, J=8.7 Hz, 1 H), 6.87-7.04 (m, 4 H), 7.14 (d, J=7.8 Hz, 1 H), 7.20-7.30 (m, 4 H), 7.34-7.39 (m, 2 H), 7.44-7.48 (m, 2 H), 7.60 (d, J=7.9 Hz, 1 H), 8.54 (s, 1 H), 9.83 (s, 1 H), 10.82 (d, J=2.1 Hz, 1 H).

LC/MS (ESI) m/z 582 (M+H⁺).

### Example 151

### N-[(1R,2S)-1-[({5-[(dimethylamino)methyl]-2-ethoxyphenyl}amino)carbonyl]-2-(1H-indol-3-yl)propyl]-N′-phenylpiperazine-1,4-dicarboxamide

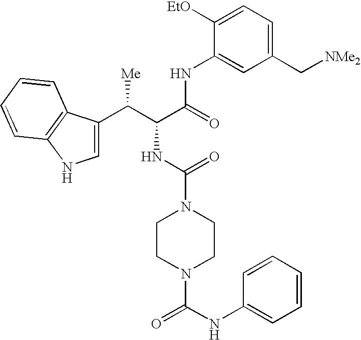

¹H NMR (300 MHz, CDCl₃) δ ppm: 1.24 (t, J=7.0 Hz, 3 H), 1.56 (t, J=7.4 Hz, 3 H), 2.20 (s, 6 H), 3.27-3.54 (m, 11 H), 3.61-3.70 (m, 1 H), 3.81-3.97 (m, 2 H), 4.85 (t, J=7.6 Hz, 1 H), 5.36 (d, J=7.6 Hz, 1 H), 6.43 (s, 1 H), 6.70 (d, J=8.5 Hz, 1 H), 6.93 (dd, J=2.1, 8.3 Hz, 1 H), 7.02-7.17 (m, 4 H), 7.26-7.37 (m, 5 H), 7.71 (d, J=7.7 Hz, 1 H), 7.93 (s, 1 H), 8.18 (d, J=2.1 Hz, 1 H), 8.24 (brs, 1 H).

LC/MS (ESI) m/z 626 (M+H⁺).

### Example 152

### N-[(1R,2S)-1-[({3-[(dimethylamino)methyl]phenyl}amino)carbonyl]-2-(1H-indol-3-yl)propyl]-4-(4-fluorophenyl)-3-oxopiperazine-1-carboxamide

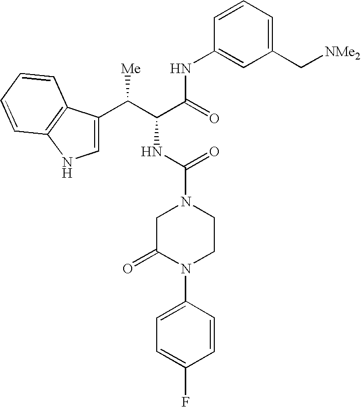

¹H NMR (300 MHz, DMSO-d₆) δ ppm: 1.32 (d, J=7.2 Hz, 3 H), 2.10 (s, 6 H), 3.27 (s, 2 H), 3.59-3.76 (m, 5 H), 4.08 (d, J=17.5 Hz, 1 H), 4.22 (d, J=17.5 Hz, 1 H), 4.66 (t, J=8.5 Hz, 1 H), 6.74 (d, J=8.7 Hz, 1 H), 6.89 (d, J=7.7 Hz, 1 H), 6.92-6.98 (m, 1 H), 7.00-7.05 (m, 1 H), 7.14 (d, J=7.7 Hz, 1 H), 7.20-7.39 (m, 8 H), 7.62 (d, J=7.7 Hz, 1 H), 9.82 (s, 1 H), 10.81 (d, J=1.7 Hz, 1 H).

LC/MS (ESI) m/z 571 (M+H⁺).

### Example 153

### N-[(1R,2S)-1-[({5-[(dimethylamino)methyl]-2-ethoxyphenyl}amino)carbonyl]-2-(1H-indol-3-yl)propyl]-4-(4-fluorophenyl)-3-oxopiperazine-1-carboxamide

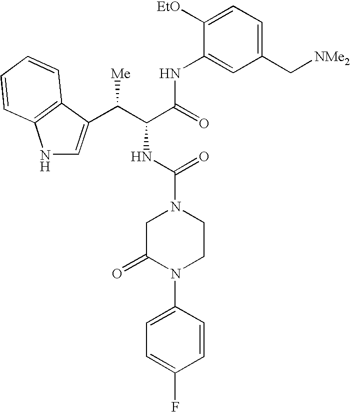

¹H NMR (300 MHz, CDCl₃) δ ppm: 1.25 (t, J=6.9 Hz, 3 H), 1.57 (d, J=7.4 Hz, 3 H), 2.21 (s, 6 H), 3.32 (s, 2 H), 3.61-3.97 (m, 7 H), 4.09 (d, J=17.3 Hz, 1 H), 4.21 (d, J=17.3 Hz, 1 H), 4.86 (t, J=7.5 Hz, 1 H), 5.32 (d, J=7.7 Hz, 1 H), 6.70 (d, J=8.4 Hz, 1 H), 6.94 (dd, J=2.1, 8.4 Hz, 1 H), 7.05-7.19 (m, 5 H), 7.21-7.27 (m, 2 H), 7.31-7.34 (m, 1 H), 7.73 (d, J=7.7 Hz, 1 H), 7.87 (s, 1 H), 8.11 (brs, 1 H), 8.18 (d, J=2.1 Hz, 1 H).

LC/MS (ESI) m/z 615 (M+H⁺).

### Example 154

### N-[(1R,2S)-1-[({3-[(dimethylamino)methyl]phenyl}amino)carbonyl]-2-(1H-indol-3-yl)propyl]-4-(2-methylphenyl)-3-oxopiperazine-1-carboxamide

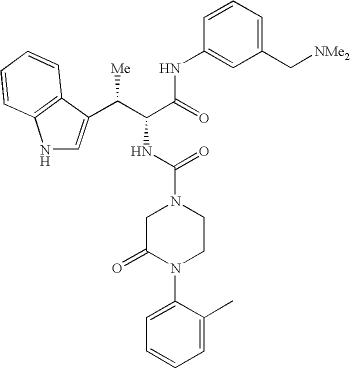

¹H NMR (300 MHz, DMSO-d₆) δ ppm: 1.34 (d, J=7.0 Hz, 3 H), 2.06 (d, J=8.3 Hz, 3 H), 2.11 (s, 6 H), 3.27 (s, 2 H), 3.32-3.46 (m, 1 H), 3.53-3.88 (m, 4 H), 4.07 (dd, J=11.6, 17.5 Hz, 1 H), 4.22 (dd, J=7.1, 17.5 Hz, 1 H), 4.67 (t, J=8.6 Hz, 1 H), 6.78-6.82 (m, 1 H), 6.89 (d, J=7.7 Hz, 1 H), 6.93-6.98 (m, 1 H), 6.99-7.04 (m, 1 H), 7.12-7.38 (m, 9 H), 7.62 (d, J=7.7 Hz, 1 H), 9.83 (s, 1 H), 10.81 (s, 1 H).

LC/MS (ESI) m/z 567 (M+H⁺).

### Example 155

### N-[(1R,2S)-1-[({5-[(dimethylamino)methyl]-2-ethoxyphenyl}amino)carbonyl]-2-(1H-indol-3-yl)propyl]-4-(2-methylphenyl)-3-oxopiperazine-1-carboxamide

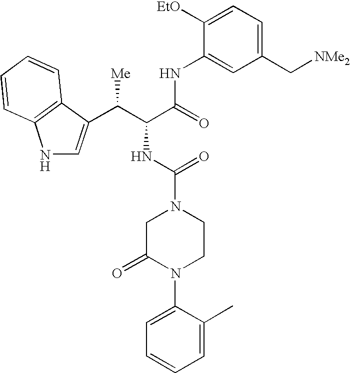

¹H NMR (300 MHz, CDCl₃) δ ppm: 1.25 (t, J=7.0 Hz, 3 H), 1.57 (d, J=7.2 Hz, 3 H), 2.21 (s, 9 H), 3.33 (s, 2 H), 3.47-3.56 (m, 1 H), 3.62-3.97 (m, 6 H), 4.10 (d, J=17.1 Hz, 1 H), 4.22 (dd, J=2.5, 17.1 Hz), 4.87 (t, J=7.4 Hz, 1 H), 5.32 (d, J=7.7 Hz, 1 H), 6.70 (d, J=8.3 Hz, 1 H), 6.94 (dd, J=2.0, 8.3 Hz, 1 H), 7.08-7.19 (m, 4 H), 7.23-7.29 (m, 3 H), 7.33 (d, J=7.9 Hz, 1 H), 7.74 (d, J=7.5 Hz, 1 H), 7.90 (s, 1 H), 8.10 (brs, 1 H), 8.18 (d, J=2.1 Hz, 1 H).

LC/MS (ESI) m/z 611 (M+H⁺).

### Example 156

### N-[(1R,2S)-1-[({3-[(dimethylamino)methyl]phenyl}amino)carbonyl]-2-(1H-indol-3-yl)propyl]-4-(4-fluoro-2-methylphenyl)-3-oxopiperazine-1-carboxamide

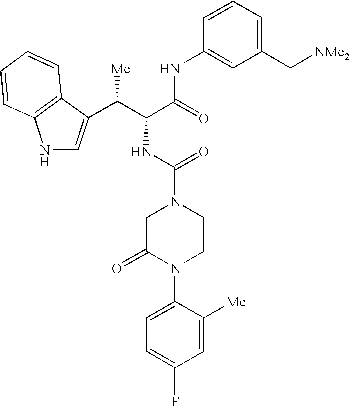

¹H NMR (300 MHz, DMSO-d₆) δ ppm: 1.34 (d, J=7.2 Hz, 3 H), 2.05 (d, J=12.1 Hz, 3 H), 2.11 (s, 6 H), 3.27 (s, 2 H), 3.30-3.86 (m, 5 H), 4.06 (dd, J=10.8, 17.6 Hz, 1 H), 4.22 (dd, J=2.9, 17.6 Hz, 1 H), 4.67 (t, J=8.5 Hz, 1 H), 6.78-6.82 (m, 1 H), 6.88-7.38 (m, 11 H), 7.62 (d, J=7.9 Hz, 1 H), 9.83 (d, J=2.3 Hz, 1 H), 10.80 (s, 1 H).

LC/MS (ESI) m/z 585 (M+H⁺).

### Example 157

### N-[(1R,2S)-1-[({5-[(dimethylamino)methyl]-2-ethoxyphenyl}amino)carbonyl]-2-(1H-indol-3-yl)propyl]-4-(4-fluoro-2-methylphenyl)-3-oxopiperazine-1-carboxamide

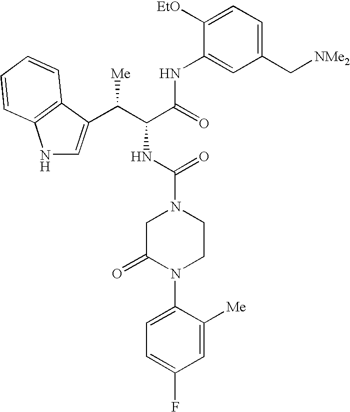

¹H NMR (300 MHz, CDCl₃) δ ppm: 1.25 (t, J=7.0 Hz, 3 H), 1.57 (d, J=7.4 Hz, 3 H), 2.19 (d, J=1.7 Hz, 3 H), 2.21 (s, 6 H), 3.33 (s, 2 H), 3.44-3.53 (m, 1 H), 3.58-3.75 (m, 3 H), 3.78-3.97 (m, 3 H), 4.10 (dd, J=1.5, 17.3 Hz, 1 H), 4.21 (dd, J=4.1, 17.3 Hz, 1 H), 4.87 (t, J=7.4 Hz, 1 H), 5.34 (d, J=7.9 Hz, 1 H), 6.70 (d, J=8.5 Hz, 1 H), 6.91-7.01 (m, 3 H), 7.05-7.12 (m, 3 H), 7.14-7.19 (m, 1 H), 7.33 (d, J=7.7 Hz, 1 H), 7.73 (d, J=7.7 Hz, 1 H), 7.87 (s, 1 H), 8.09 (brs, 1 H), 8.18 (d, J=2.1 Hz, 1 H).

LC/MS (ESI) m/z 629 (M+H⁺).

### Example 158

### N-[(1R,2S)-1-[({3-[(dimethylamino)methyl]phenyl}amino)carbonyl]-2-(1H-indol-3-yl)propyl]-3,5-dioxo-4-phenylpiperazine-1-carboxamide

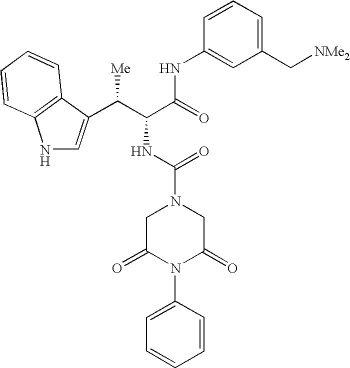

¹H NMR (300 MHz, DMSO-d₆) δ ppm: 1.32 (d, J=7.2 Hz, 3 H), 2.11 (s, 6 H), 3.29 (s, 2 H), 3.58-3.68 (m, 1 H), 4.47 (d, J=17.9 Hz, 2 H), 4.54 (d, J=17.9 Hz, 2 H), 4.67 (t, J=8.6 Hz, 1 H), 6.89 (d, J=7.5 Hz, 1 H), 6.93-7.00 (m, 1 H), 7.03-7.09 (m, 3 H), 7.12-7.17 (m, 1 H), 7.26 (d, J=7.7 Hz, 1 H), 7.31 (d, J=2.3 Hz, 1 H), 7.34-7.45 (m, 6 H), 7.62 (d, J=7.9 Hz, 1 H), 9.88 (s, 1 H), 10.82 (d, J=1.9 Hz, 1 H).

LC/MS (ESI) m/z 567 (M+H⁺).

### Example 159

### 4-(4-chlorophenyl)-N-[(1R,2S)-1-[({5-[(dimethylamino)methyl]-2-methoxyphenyl}amino)carbonyl]-2-(1H-indol-3-yl)propyl]piperazine-1-carboxamide

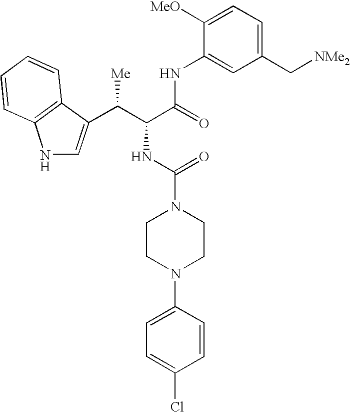

A mixture of (2R,3S)-2-({[4-(4-chlorophenyl)piperazin-1-yl]carbonyl}amino)-3-(1H-indol-3-yl)butanoic acid (220 mg, 0.500 mmol), 5-dimethylaminomethyl-2-methoxyaniline dihydrochloride (152 mg, 0.600 mmol), WSC (115 mg, 0.600 mmol), HOBt (91.9 mg, 0.600 mmol), triethylamine (0.167 mL, 2.40 mmol), THF (1 mL) and acetonitrile (1 mL) was stirred at room temperature for 2 hrs. To the reaction mixture were added ethyl acetate (5 mL) and saturated aqueous solution of sodium hydrogen carbonate (5 mL) and the mixture was further stirred for 10 min. The organic layer was dried (MgSO₄), passed through an aminopropyl silica gel layer and concentrated under reduced pressure. The residue was crystallized from hexane/ethyl acetate to give the title compound as a colorless crystalline powder (189 mg, yield 65%).

¹H NMR (300 MHz, CDCl₃) δ ppm: 1.57 (d, J=7.2 Hz, 3 H) , 2.19 (s, 6 H), 3.06-3.18 (m, 4 H), 3.30 (s, 2 H), 3.46-3.61 (m, 8 H), 4.93 (t, J=8.1 Hz, 1 H), 5.43 (d, J=8.1 Hz, 1 H), 6.65 (d, J=8.3 Hz, 1 H), 6.80-6.85 (m, 2 H), 6.92 (dd, J=2.1, 8.3 Hz, 1 H), 7.07-7.25 (m, 5 H), 7.32 (d, J=7.7 Hz, 1 H), 7.68 (s, 1 H), 7.78 (d, J=7.7 Hz, 1 H), 8.05 (brs, 1 H), 8.11 (d, J=2.1 Hz, 1 H).

LC/MS (ESI) m/z 603 (M+H⁺).

The compounds described in the following Examples 160-180 were produced in the similar manner as in Example 159.

### Example 160

### 4-(4-chlorophenyl)-N-[(1R,2S)-1-[({5-[(dimethylamino)methyl]-2-ethoxyphenyl}amino)carbonyl]-2-(1H-indol-3-yl)propyl]piperazine-1-carboxamide

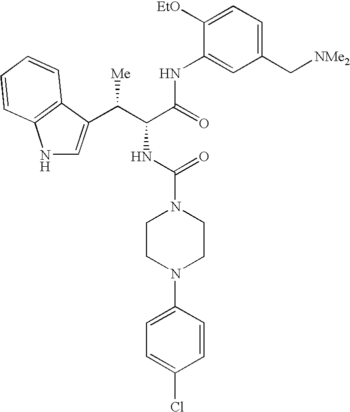

¹H NMR (300 MHz, CDCl₃) δ ppm: 1.23 (t, J=7.0 Hz, 3 H), 1.58 (d, J=7.2 Hz, 3 H), 2.20 (s, 6 H), 3.03-3.15 (m, 4 H), 3.32 (s, 2 H), 3.42-3.67 (m, 5 H), 3.79-3.96 (m, 2 H), 4.89 (dd, J=7.2, 7.9 Hz, 1 H), 5.38 (d, J=7.9 Hz, 1 H), 6.70 (d, J=8.3 Hz, 1 H), 6.79-6.84 (m, 2 H), 6.93 (dd, J=2.1, 8.3 Hz, 1 H), 7.04-7.10 (m, 2 H), 7.12-7.17 (m, 1 H), 7.19-7.24 (m, 2 H), 7.31 (d, J=7.9 Hz, 1 H), 7.73 (d, J=7.7 Hz, 1 H), 7.86 (s, 1 H), 8.07 (brs. 1 H), 8.17 (d, J=2.1 Hz, 1 H).

LC/MS (ESI) m/z 617 (M+H⁺).

### Example 161

### 4-(2-chlorophenyl)-N-[(1R,2S)-1-[({5-[(dimethylamino)methyl]-2-methoxyphenyl}amino)carbonyl]-2-(1H-indol-3-yl)propyl]piperazine-1-carboxamide

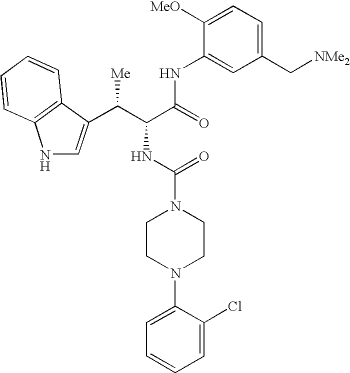

¹H NMR (300 MHz, CDCl₃) δ ppm: 1.57 (d, J=7.2 Hz, 3 H), 2.19 (s, 6 H), 2.95-3.07 (m, 4 H), 3.31 (s, 2 H), 3.50-3.64 (m, 8 H), 4.94 (t, J=8.1 Hz, 1 H), 5.41 (d, J=8.1 Hz, 1 H), 6.65 (d, J=8.3 Hz, 1 H), 6.92 (dd, J=2.1, 8.3 Hz, 1 H), 6.97-7.03 (m, 2 H), 7.07-7.23 (m, 4 H), 7.31-7.34 (m, 1 H), 7.38 (dd, J=1.5, 8.3 Hz, 1 H), 7.72 (s, 1 H), 7.79 (d, J=7.5 Hz, 1 H), 8.06 (brs, 1 H), 8.12 (d, J=1.9 Hz, 1 H).

LC/MS (ESI) m/z 603 (M+H⁺).

### Example 162

### N-[(1R,2S)-1-[({5-[(dimethylamino)methyl]-2-methoxyphenyl}amino)carbonyl]-2-(1H-indol-3-yl)propyl]-4-(4-methylphenyl)piperazine-1-carboxamide

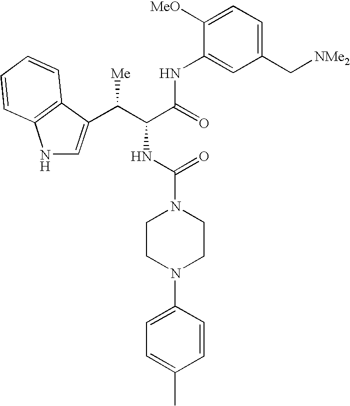

¹H NMR (300 MHz, CDCl₃) δ ppm: 1.57 (d, J=7.4 Hz, 3 H), 2.19 (s, 6 H), 2.28 (s, 3 H), 3.04-3.16 (m, 4 H), 3.30 (s, 2 H), 3.46-3.61 (m, 8 H), 4.93 (t, J=8.1 Hz, 1 H), 5.42 (d, J=8.1 Hz, 1 H), 6.65 (d, J=8.3 Hz, 1 H), 6.81-6.86 (m, 2 H), 6.92 (dd, J=2.1, 8.3 Hz, 1 H), 7.06-7.19 (m, 5 H), 7.32 (d, J=7.5 Hz, 1 H), 7.71 (s, 1 H), 7.76 (d, J=7.7 Hz, 1 H), 8.08 (brs, 1 H), 8.12 (d, J=2.1 Hz, 1 H).

LC/MS (ESI) m/z 583 (M+H⁺).

### Example 163

### N-[(1R,2S)-1-[({5-[(dimethylamino)methyl]-2-ethoxyphenyl}amino)carbonyl]-2-(1H-indol-3-yl)propyl]-4-(4-methylphenyl)piperazine-1-carboxamide

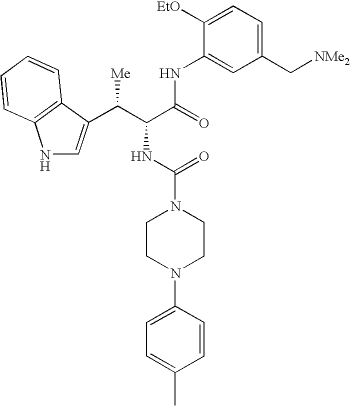

¹H NMR (300 MHz, CDCl₃) δ ppm: 1.23 (t, J=7.0 Hz, 3 H), 1.58 (d, J=7.2 Hz, 3 H), 2.20 (s, 6 H), 2.28 (s, 3 H), 3.02-3.13 (m, 4 H), 3.32 (s, 2 H), 3.42-3.68 (m, 5 H), 3.79-3.95 (m, 2 H), 4.89 (t, J=7.4 Hz, 1 H), 5.35 (d, J=7.7 Hz, 1 H), 6.69 (d, J=8.3 Hz, 1 H), 6.81-6.85 (m, 2 H), 6.93 (dd, J=2.1, 8.3 Hz, 1 H), 7.06-7.17 (m, 5 H), 7.31 (d, J=8.1 Hz, 1 H), 7.74 (d, J=7.7 Hz, 1 H), 7.90 (s, 1 H), 8.05 (brs. 1 H), 8.18 (d, J=2.1 Hz, 1 H).

LC/MS (ESI) m/z 597 (M+H⁺).

### Example 164

### N-[(1R,2S)-1-[({5-[(dimethylamino)methyl]-2-methoxyphenyl}amino)carbonyl]-2-(1H-indol-3-yl)propyl]-4-[4-(trifluoromethyl)phenyl]piperazine-1-carboxamide

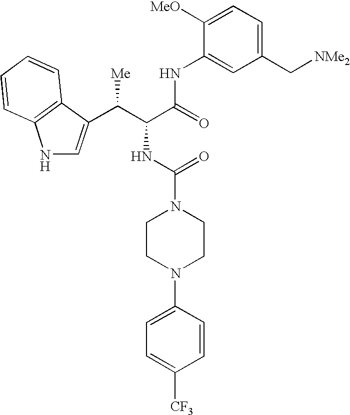

¹H NMR (300 MHz, CDCl₃) δ ppm: 1.57 (d, J=7.2 Hz, 3 H), 2.19 (s, 6 H), 3.21-3.34 (m, 6 H), 3.48-3.63 (m, 8 H), 4.93 (t, J=8.1 Hz, 1 H), 5.45 (d, J=8.1 Hz, 1 H), 6.65 (d, J=8.5 Hz, 1 H), 6.88-6.94 (m, 3 H), 7.07-7.12 (m, 2 H), 7.14-7.19 (m, 1 H), 7.33 (d, J=7.7 Hz, 1 H), 7.47-7.52 (m, 2 H), 7.67 (s, 1 H), 7.78 (d, J=7.9 Hz, 1 H), 8.08 (brs, 1 H), 8.11 (d, J=2.1 Hz, 1 H).

LC/MS (ESI) m/z 637 (M+H⁺).

### Example 165

### N-[(1R,2S)-1-[({5-[(dimethylamino)methyl]-2-ethoxyphenyl}amino)carbonyl]-2-(1H-indol-3-yl)propyl]-4-[4-(trifluoromethyl)phenyl]piperazine-1-carboxamide

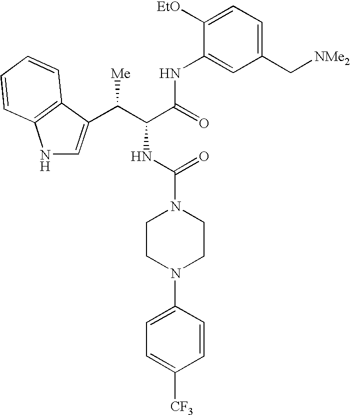

¹H NMR (300 MHz, CDCl₃) δ ppm: 1.23 (t, J=7.0 Hz, 3 H), 1.58 (d, J=7.2 Hz, 3 H), 2.20 (s, 6 H), 3.19-3.31 (m, 4 H), 3.32 (s, 2 H), 3.44-3.67 (m, 5 H), 3.79-3.96 (m, 2 H), 4.89 (dd, J=7.2, 7.9 Hz, 1 H), 5.38 (d, J=7.9 Hz, 1 H), 6.69 (d, J=8.3 Hz, 1 H), 6.87-6.95 (m, 3 H), 7.04-7.10 (m, 2 H), 7.12-7.17 (m, 1 H), 7.32 (d, J=7.9 Hz, 1 H), 7.47-7.51 (m, 2 H), 7.73 (d, J=7.9 Hz, 1 H), 7.85 (s, 1 H), 8.05 (brs, 1 H), 8.17 (d, J=2.1 Hz, 1 H).

LC/MS (ESI) m/z 651 (M+H⁺).

### Example 166

### N-[(1R,2S)-1-[({5-[(dimethylamino)methyl]-2-methoxyphenyl}amino)carbonyl]-2-(1H-indol-3-yl)propyl]-4-(4-methoxyphenyl)piperazine-1-carboxamide

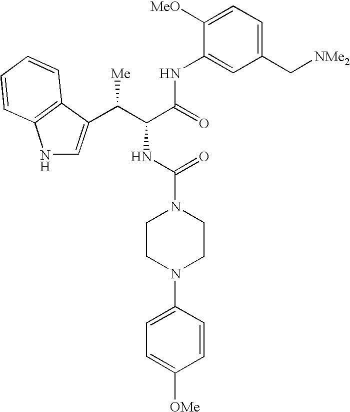

¹H NMR (300 MHz, CDCl₃) δ ppm: 1.57 (d, J=7.2 Hz, 3 H), 2.19 (s, 6 H), 2.97-3.09 (m, 4 H), 3.30 (s, 2 H), 3.46-3.61 (m, 8 H), 3.78 (s, 3 H), 4.93 (t, J=8.1 Hz, 1 H), 5.41 (d, J=7.9 Hz, 1 H), 6.65 (d, J=8.3 Hz, 1 H), 6.82-6.94 (m, 5 H), 7.07 (d, J=2.6 Hz, 1 H), 7.10-7.19 (m, 2 H), 7.32 (d, J=7.5 Hz, 1 H), 7.71 (s, 1 H), 7.78 (d, J=7.5 Hz, 1 H), 8.07 (brs, 1 H), 8.12 (d, J=2.1 Hz, 1 H).

LC/MS (ESI) m/z 599 (M+H⁺).

### Example 167

### N-[(1R,2S)-1-[({5-[(dimethylamino)methyl]-2-ethoxyphenyl}amino)carbonyl]-2-(1H-indol-3-yl)propyl]-4-(4-methoxyphenyl)piperazine-1-carboxamide

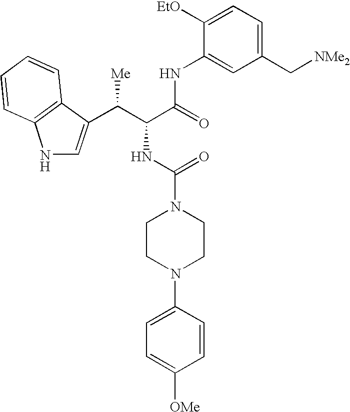

¹H NMR (300 MHz, CDCl₃) δ ppm: 1.22 (t, J=7.0 Hz, 3 H), 1.58 (d, J=7.2 Hz, 3 H), 2.20 (s, 6 H), 2.95-3.07 (m, 4 H), 3.32 (s, 2 H), 3.42-3.71 (m, 5 H), 3.78 (s, 3 H), 3.80-3.95 (m, 2 H), 4.89 (t, J=7.4 Hz, 1 H), 5.36 (d, J=7.9 Hz, 1 H), 6.69 (d, J=8.3 Hz, 1 H), 6.82-6.94 (m, 5 H), 7.05-7.10 (m, 2 H), 7.12-7.17 (m, 1 H), 7.31 (d, J -7.7 Hz, 1 H), 7.74 (d, J=7.9 Hz, 1 H), 7.90 (s, 1 H), 8.07 (brs, 1 H), 8.18 (d, J=2.1 Hz, 1 H).

LC/MS (ESI) m/z 613 (M+H⁺).

### Example 168

### N-[(1R,2S)-1-[({5-[(dimethylamino)methyl]-2-methoxyphenyl}amino)carbonyl]-2-(1H-indol-3-yl)propyl]-4-(2-fluorophenyl)piperazine-1-carboxamide

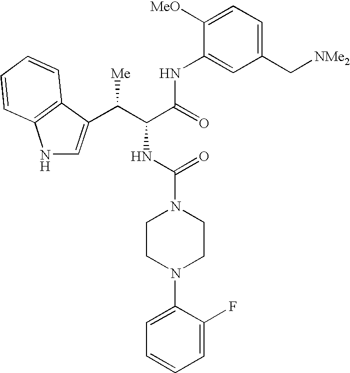

¹H NMR (300 MHz, CDCl₃) δ ppm: 1.57 (d, J=7.2 Hz, 3 H), 2.19 (s, 6H), 2.99-3.10 (m, 4 H), 3.30 (s, 2 H), 3.49-3.63 (m, 8 H), 4.94 (t, J=8.0 Hz, 1 H), 5.42 (d, J=8.0 Hz, 1 H), 6.65 (d, J=8.3 Hz, 1 H), 6.90-7.19 (m, 8 H), 7.31-7.34 (m, 1 H), 7.72 (s, 1 H), 7.79 (d, J=7.7 Hz, 1 H), 8.08 (brs, 1 H), 8.12 (d, J=2.1 Hz, 1 H).

LC/MS (ESI) m/z 587 (M+H⁺).

### Example 169

### N-[(1R,2S)-1-[({5-[(dimethylamino)methyl]-2-ethoxyphenyl}amino)carbonyl]-2-(1H-indol-3-yl)propyl]-4-(2-fluorophenyl)piperazine-1-carboxamide

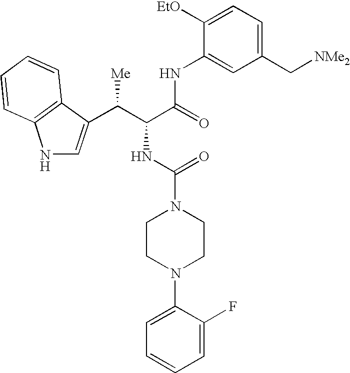

¹H NMR (300 MHz, CDCl₃) δ ppm: 1.23 (t, J=7.0 Hz, 3 H), 1.58 (d, J=7.2 Hz, 3 H), 2.20 (s, 6 H), 2.96-3.08 (m, 4 H), 3.32 (s, 2 H), 3.44-3.68 (m, 5 H), 3.79-3.96 (m, 2 H), 4.89 (t, J=7.4 Hz, 1 H), 5.36 (d, J=7.7 Hz, 1 H), 6.69 (d, J=8.5 Hz, 1 H), 6.89-7.18 (m, 8 H), 7.30-7.33 (m, 1 H), 7.74 (d, J=7.7 Hz, 1 H), 7.90 (s, 1 H), 8.06 (brs, 1 H), 8.18 (d, J=2.1 Hz, 1 H).

LC/MS (ESI) m/z 601 (M+H⁺).

### Example 170

### N-[(1R,2S)-1-[({5-[(dimethylamino)methyl]-2-methoxyphenyl}amino)carbonyl]-2-(1H-indol-3-yl)propyl]-4-(4-fluoro-2-methylphenyl)piperazine-1-carboxamide

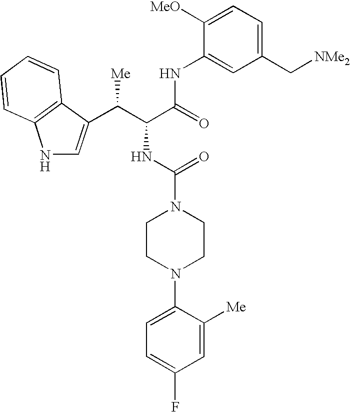

¹H NMR (300 MHz, CDCl₃) δ ppm: 1.58 (d, J=7.2 Hz, 3 H), 2.20 (s, 6 H), 2.30 (s, 3 H), 2.75-2.87 (m, 4 H), 3.31 (s, 2 H), 3.44-3.59 (m, 8 H), 4.94 (t, J=8.0 Hz, 1 H), 5.41 (d, J=8.0 Hz, 1 H), 6.66 (d, J=8.5 Hz, 1 H), 6.81-6.96 (m, 4 H), 7.07-7.20 (m, 3 H), 7.31-7.34 (m, 1 H), 7.70 (s, 1 H), 7.80 (d, J=7.4 Hz, 1 H), 8.03 (brs, 1 H), 8.12 (d, J=2.1 Hz, 1 H).

LC/MS (ESI) m/z 601 (M+H⁺).

### Example 171

### N-[(1R,2S)-1-[({5-[(dimethylamino)methyl]-2-ethoxyphenyl}amino)carbonyl]-2-(1H-indol-3-yl)propyl]-4-(4-fluoro-2-methylphenyl)piperazine-1-carboxamide

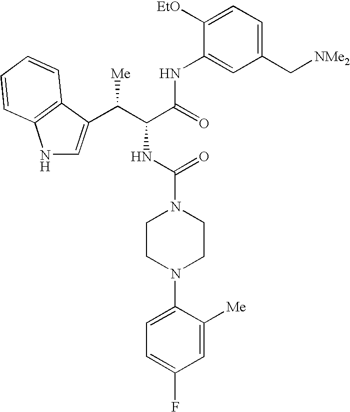

¹H NMR (300 MHz, CDCl₃) δ ppm: 1.23 (t, J=7.1 Hz, 3 H), 1.58 (d, J=7.2 Hz, 3 H), 2.21 (s, 6 H), 2.29 (s, 3 H), 2.73-2.85 (m, 4 H), 3.32 (s, 2 H), 3.40-3.57 (m, 4 H), 3.59-3.68 (m, 1 H), 3.79-3.96 (m, 2 H), 4.90 (t, J=7.5 Hz, 1 H), 5.36 (d, J=7.9 Hz, 1 H), 6.69 (d, J=8.3 Hz, 1 H), 6.81-6.95 (m, 4 H), 7.07-7.18 (m, 3 H), 7.32 (d, J=7.9 Hz, 1 H), 7.75 (d, J=7.7 Hz, 1 H), 7.89 (s, 1 H), 8.03 (brs, 1 H), 8.18 (d, J=2.1 Hz, 1 H).

LC/MS (ESI) m/z 615 (M+H⁺).

### Example 172

### 4-(3-chlorophenyl)-N-[(1R,2S)-1-[({5-[(dimethylamino)methyl]-2-methoxyphenyl}amino)carbonyl]-2-(1H-indol-3-yl)propyl]piperazine-1-carboxamide

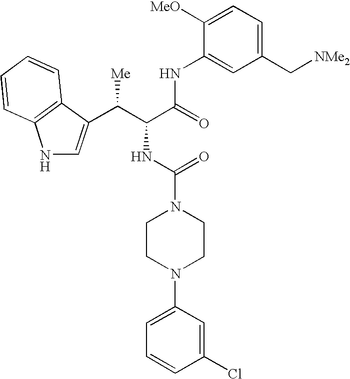

¹H NMR (300 MHz, CDCl₃) δ ppm: 1.57 (d, J=7.4 Hz, 3 H), 2.19 (s, 6 H), 3.10-3.22 (m, 4 H), 3.30 (s, 2 H), 3.45-3.61 (m, 8 H), 4.92 (t, J=8.1 Hz, 1 H), 5.44 (d, J=8.1 Hz, 1 H), 6.65 (d, J=8.3 Hz, 1 H), 6.75-6.78 (m, 1 H), 6.83-6.87 (m, 2 H), 6.92 (dd, J=2.1, 8.3 Hz, 1 H), 7.07 (d, J=2.5 Hz, 1 H), 7.09-7.20 (m, 3 H), 7.31-7.34 (m, 1 H), 7.69 (s, 1 H), 7.78 (d, J=7.7 Hz, 1 H), 8.10 (brs, 1 H), 8.11 (d, J=2.1 Hz, 1 H).

LC/MS (ESI) m/z 603 (M+H⁺).

### Example 173

### N-[(1R,2S)-1-[({5-[(dimethylamino)methyl]-2-methoxyphenyl}amino)carbonyl]-2-(1H-indol-3-yl)propyl]-4-(3-fluorophenyl)piperazine-1-carboxamide

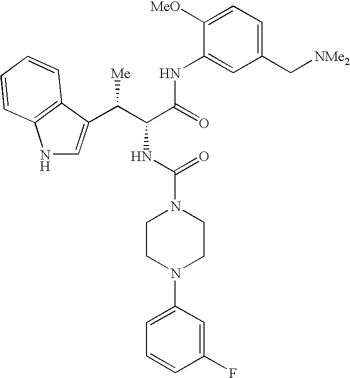

¹H NMR (300 MHz, CDCl₃) δ ppm: 1.57 (d, J=7.2 Hz, 3 H), 2.23 (s, 6 H), 3.11-3.23 (m, 4 H), 3.35 (s, 2 H), 3.46-3.61 (m, 8 H), 4.92 (t, J=8.0 Hz, 1 H), 5.42 (d, J=8.0 Hz, 1 H), 6.53-6.60 (m, 2 H), 6.64-6.68 (m, 2 H), 6.95 (dd, J=2.0, 8.4 Hz, 1 H), 7.07-7.24 (m, 4 H), 7.33 (d, J=7.5 Hz, 1 H), 7.69 (s, 1 H), 7.77 (d, J=7.9 Hz, 1 H), 8.09 (brs, 1 H), 8.11 (d, J=1.9 Hz, 1 H).

LC/MS (ESI) m/z 587 (M+H⁺).

### Example 174

### N-[(1R,2S)-1-[({5-[(dimethylamino)methyl]-2-ethoxyphenyl}amino)carbonyl]-2-(1H-indol-3-yl)propyl]-4-(3-fluorophenyl)piperazine-1-carboxamide

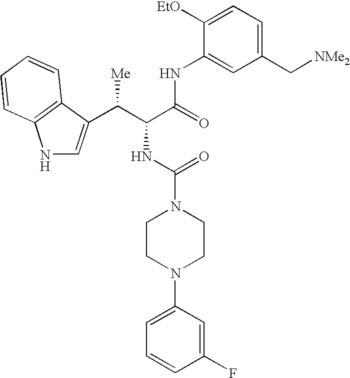

¹H NMR (300 MHz, CDCl₃) δ ppm: 1.23 (t, J=7.0 Hz, 3 H) , 1.58 (d, J=7.4 Hz, 3 H), 2.23 (s, 6 H), 3.09-3.21 (m, 4 H), 3.35 (s, 2 H), 3.42-3.68 (m, 5 H), 3.80-3.96 (m, 2 H), 4.88 (t, J=7.6 Hz, 1 H), 5.37 (d, J=7.6 Hz, 1 H), 6.53-6.59 (m, 2 H), 6.63-6.67 (m, 1 H), 6.70 (d, J=8.4 Hz, 1 H), 6.95 (dd, J=2.0, 8.4 Hz, 1 H), 7.04-7.24 (m, 4 H), 7.32 (d, J=7.9 Hz, 1 H), 7.73 (d, J=7.7 Hz, 1 H), 7.88 (s, 1 H), 8.11 (brs, 1 H), 8.17 (d, J=2.1 Hz, 1 H).

LC/MS (ESI) m/z 601 (M+H⁺).

### Example 175

### 4-(cyclopropylmethyl)-N-[(1R,2S)-1-[({5-[(dimethylamino)methyl]-2-methoxyphenyl}amino)carbonyl]-2-(1H-indol-3-yl)propyl]piperazine-1-carboxamide

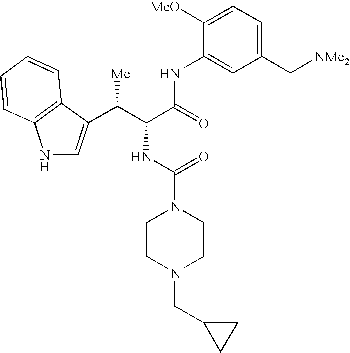

¹H NMR (300 MHz, CDCl₃) δ ppm: 0.08-0.13 (m, 2 H), 0.50-0.56 (m, 2 H), 0.79-0.92 (m, 1 H), 1.55 (d, J=7.2 Hz, 3 H), 2.19 (s, 6 H), 2.26 (d, J=6.4 Hz, 2 H), 2.44-2.55 (m, 4 H), 3.30-3.55 (m, 10 H), 4.91 (t, J=8.1 Hz, 1 H), 5.35 (d, J=8.1 Hz, 1 H), 6.65 (d, J=8.3 Hz, 1 H), 6.91 (dd, J=2.1, 8.3 Hz, 1 H), 7.05-7.19 (m, 3 H), 7.31 (d, J=7.5 Hz, 1 H), 7.72 (s, 1 H), 7.78 (d, J=7.7 Hz, 1 H), 8.08 (s, 1 H), 8.10 (d, J=1.9 Hz, 1 H).

LC/MS (ESI) m/z 547 (M+H⁺).

### Example 176

### N-[(1R,2S)-1-[({5-[(dimethylamino)methyl]-2-methoxyphenyl}amino)carbonyl]-2-(1H-indol-3-yl)propyl]-N′-phenylpiperazine-1,4-dicarboxamide

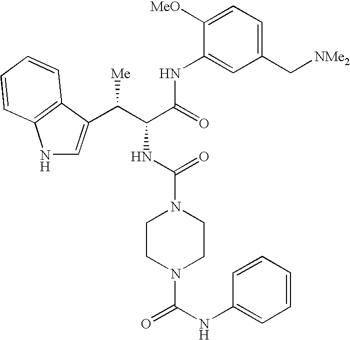

¹H NMR (300 MHz, CDCl₃) δ ppm: 1.56 (t, J=7.2 Hz, 3 H), 2.19 (s, 6 H), 3.29 (s, 2 H), 3.37-3.61 (m, 12 H), 4.90 (t, J=8.0 Hz, 1 H), 5.44 (d, J=8.0 Hz, 1 H), 6.44 (s, 1 H), 6.66 (d, J=8.3 Hz, 1 H), 6.92 (dd, J=2.1, 8.3 Hz, 1 H), 7.02-7.19 (m, 4 H), 7.27-7.39 (m, 5 H), 7.72 (s, 1 H), 7.75 (d, J=7.7 Hz, 1 H), 8.12 (d, J=2.1 Hz, 1 H), 8.21 (brs, 1 H).

LC/MS (ESI) m/z 612 (M+H⁺).

### Example 177

### N-[(1R,2S)-1-[({5-[(dimethylamino)methyl]-2-methoxyphenyl}amino)carbonyl]-2-(1H-indol-3-yl)propyl]-4-(4-fluorophenyl)-3-oxopiperazine-1-carboxamide

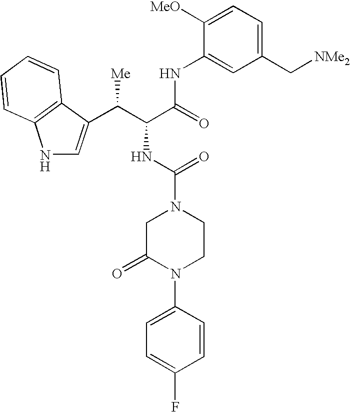 [2032] ¹H NMR (300 MHz, CDCl₃) δ ppm: 1.57 (d, J=7.2 Hz, 3 H), 2.20 (s, 6 H), 3.31 (s, 2 H), 3.51-3.60 (m, 4 H), 3.65-3.85 (m, 4 H) , 4.15 (d, J=17.3 Hz, 1 H), 4.23 (d, J=17.3 Hz, 1 H), 4.90 (t, J=8.1 Hz, 1 H), 5.40 (d, J=7.9 Hz, 1 H), 6.66 (d, J=8.4 Hz, 1 H), 6.92 (dd, J=1.9, 8.4 Hz, 1 H), 7.06-7.20 (m, 5 H), 7.21-7.27 (m, 2 H), 7.33 (d, J=7.7 Hz, 1 H), 7.70 (s, 1 H), 7.77 (d, J=7.7 Hz, 1 H), 8.11 (d, J=1.9 Hz, 1 H), 8.14 (brs, 1 H).

LC/MS (ESI) m/z 601 (M+H⁺).

### Example 178

### N-[(1R,2S)-1-[({5-[(dimethylamino)methyl]-2-methoxyphenyl}amino)carbonyl]-2-(1H-indol-3-yl)propyl]-4-(2-methylphenyl)-3-oxopiperazine-1-carboxamide

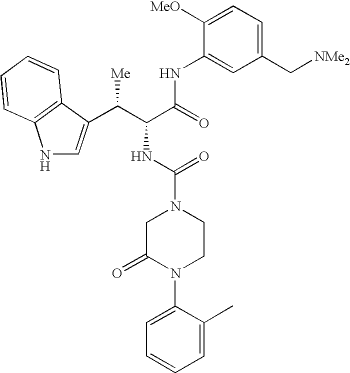 [2035] ¹H NMR (300 MHz, CDCl₃) δ ppm: 1.57 (d, J=7.2 Hz, 3 H), 2.20 (s, 6 H), 2.21 (s, 3 H), 3.31 (s, 2 H), 3.48-3.95 (m, 8 H), 4.16 (d, J=17.1 Hz, 1 H), 4.25 (dd, J=2.8, 17.1 Hz, 1 H), 4.92 (t, J=8.1 Hz, 1 H), 5.42 (d, J=7.9 Hz, 1 H), 6.66 (d, J=8.3 Hz, 1 H), 6.92 (dd, J=2.1, 8.3 Hz, 1 H), 7.08-7.20 (m, 4 H), 7.23-7.29 (m, 3 H), 7.31-7.34 (m, 1 H), 7.72 (s, 1 H), 7.78 (d, J=7.5 Hz, 1 H), 8.12 (d, J=2.1 Hz, 1 H), 8.16 (s, 1 H).

LC/MS (ESI) m/z 597 (M+H⁺).

### Example 179

### N-[(1R,2S)-1-[({5-[(dimethylamino)methyl]-2-methoxyphenyl}amino)carbonyl]-2-(1H-indol-3-yl)propyl]-4-(4-fluoro-2-methylphenyl)-3-oxopiperazine-1-carboxamide

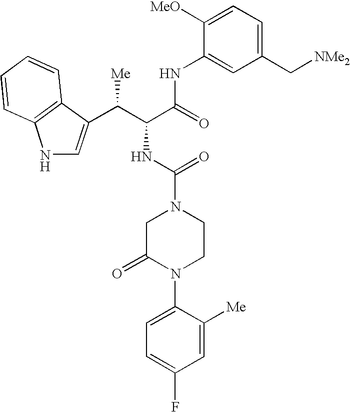

¹H NMR (300 MHz, CDCl₃) δ ppm: 1.58 (d, J=7.2 Hz, 3 H), 2.20 (s, 9 H), 3.31 (s, 2 H), 3.45-3.94 (m, 8 H), 4.11-4.28 (m, 2 H), 4.91 (t, J=8.1 Hz, 1 H), 5.43 (d, J=7.9 Hz, 1 H), 6.66 (d, J=8.3 Hz, 1 H), 6.91-7.01 (m, 3 H), 7.06-7.20 (m, 4 H), 7.33 (d, J=7.7 Hz, 1 H), 7.69 (s, 1 H), 7.77 (d, J=7.7 Hz, 1 H), 8.11 (d, J=2.1 Hz, 1 H), 8.13 (s, 1 H).

LC/MS (ESI) m/z 615 (M+H⁺).

### Example 180

### N-[(1R,2S)-1-[({5-[(dimethylamino)methyl]-2-ethoxyphenyl}amino)carbonyl]-2-(1H-indol-3-yl)propyl]-3,5-dioxo-4-phenylpiperazine-1-carboxamide

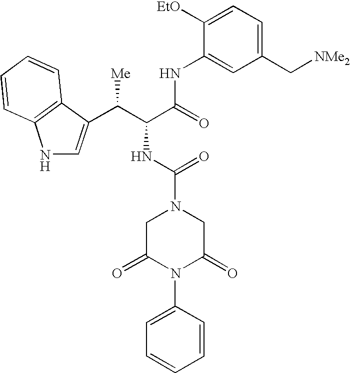

¹H NMR (300 MHz, CDCl₃) δ ppm: 1.25 (t, J=7.0 Hz, 3 H), 1.58 (d, J=7.2 Hz, 3 H), 2.21 (s, 6 H), 3.33 (s, 2 H), 3.60-3.69 (m, 1 H), 3.80-3.98 (m, 2 H), 4.33 (d, J=18.1 Hz, 2 H), 4.46 (d, J=18.1 Hz, 2 H), 4.85 (t, J=7.7 Hz, 1 H), 5.65 (d, J=7.7 Hz, 1 H), 6.70 (d, J=8.3 Hz, 1 H), 6.93 (dd, J=2.0, 8.3 Hz, 1 H), 7.05-7.11 (m, 4 H), 7.13-7.19 (m, 1 H), 7.32 (d, J=7.9 Hz, 1 H), 7.40-7.50 (m, 3 H), 7.70 (d, J=7.7 Hz, 1 H), 7.81 (s, 1 H), 8.16 (d, J=1.7 Hz, 1 H), 10 8.17 (brs, 1 H).

LC/MS (ESI) m/z 611 (M+H⁺)

### Example 181

### N-[(1R,2S)-1-[({3-[(dimethylamino)methyl]phenyl}amino)carbonyl]-2-(1H-indol-3-yl)propyl]-4-[4-fluoro-2-(hydroxymethyl)phenyl]piperazine-1-carboxamide

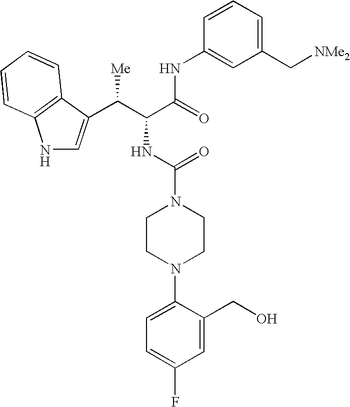

To a mixture of N-[(1R,2S)-1-[({3-[(dimethylamino)methyl])phenyl}amino)carbonyl]-2-(1H-indol-3-yl)propyl]-4-(4-fluoro-2-formylphenyl)piperazine-1-carboxamide (292 mg, 0.500 mmol), THF (5 mL) and methanol (5 mL) was added sodium borohydride (37.8 mg, 1.00 mmol) with stirring at room temperature. After 30 min., the reaction mixture was diluted with ethyl acetate and washed with water and saturated brine. The organic layer was dried (MgSO₄), passed through an aminopropyl silica gel layer and concentrated under reduced pressure. The residue was recrystallized from THF to give the title compound as a colorless crystalline powder (222 mg, yield 76%).

¹H NMR (300 MHz, DMSO-d₆) δ ppm: 1.32 (d, J=7.2 Hz, 3 H), 2.12 (s, 6 H), 2.68-2.78 (m, 4 H), 3.29 (s, 2 H), 3.39-3.54 (m, 4 H), 3.57-3.67 (m, 1 H), 4.57 (d, J=5.6 Hz, 2 H) 4.65 (t, J=8.5 Hz, 1 H), 5.23 (t, J=5.6 Hz, 1 H), 6.57 (d, J=8.5 Hz, 1 H), 6.89 (d, J=7.7 Hz, 1 H), 6.92-7.10 (m, 4 H), 7.15 (t, J=7.7 Hz, 1 H), 7.22 (dd, J=3.0, 10.0 Hz, 1 H), 7.25-7.29 (m, 2 H), 7.35-7.39 (m, 2 H), 7.61 (d, J=7.7 Hz, 1 H), 9.81 (s, 1 H), 10.80 (d, J=1.9 Hz, 1 H).

LC/MS (ESI) m/z 587 (M+H⁺).

### Example 182

### N-[(1R,2S)-1-[({3-[(dimethylamino)methyl]phenyl}amino)carbonyl]-2-(1H-indol-3-yl)propyl]-4-[4-fluoro-2-(1,3-oxazol-5-yl)phenyl]piperazine-1-carboxamide

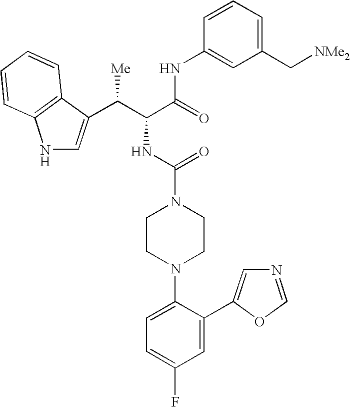

A suspension of N-[(1R,2S)-1-[({3-[(dimethylamino)methyl]phenyl}amino)carbonyl]-2-(1H-indol-3-yl)propyl]-4-(4-fluoro-2-formylphenyl)piperazine-1-carboxamide (205 mg, 0.350 mmol), p-toluenesulfonylmethylisocyanide (83.0 mg, 0.425 mmol) and potassium carbonate (72.6 mg, 0.525 mmol) in methanol (3 mL) was heated under reflux with stirring for 30 min. After cooling, the reaction mixture was diluted with ethyl acetate and washed with saturated aqueous solution of sodium hydrogen carbonate and saturated brine. The organic layer was dried (MgSO₄), passed through an aminopropyl silica gel layer and concentrated under reduced pressure. The residue was recrystallized from hexane/THF to give the title compound as a pale yellow crystalline powder (107 mg, yield 49%).

¹H NMR (300 MHz, DMSO-d₆) δ ppm: 1.33 (d, J=7.0 Hz, 3 H), 2.11 (s, 6 H), 2.71-2.82 (m, 4 H), 3.28 (s, 2 H), 3.47-3.67 (m, 5 H), 4.66 (t, J=8.5 Hz, 1 H), 6.59 (d, J=8.5 Hz, 1 H), 6.89 (d, J=7.5 Hz, 1 H), 6.93-6.98 (m, 1 H), 7.00-7.05 (m, 1 H), 7.12-7.40 (m, 7 H), 7.48 (dd, J=2.9, 9.9 Hz, 1 H), 7.61 (d, J=7.7 Hz, 1 H), 7.96 (s, 1 H), 8.49 (s, 1 H), 9.82 (s, 1 H), 10.80 (d, J=1.5 Hz, 1 H).

LC/MS (ESI) m/z 624 (M+H⁺).

### Example 183

### N-[(1R,2S)-1-[({2-(aminocarbonyl)-5-[(dimethylamino)methyl]phenyl}amino)carbonyl]-2-(1H-indol-3-yl)propyl]-4-phenylpiperidine-1-carboxamide

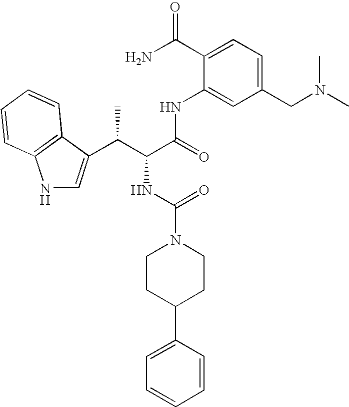

To a solution of (2R,3S)-3-(1H-indol-3-yl)-2-{[(4-phenylpiperidin-1-yl)carbonyl]amino}butanoic acid (101 mg, 0.25 mmol) and 2-amino-4-[(dimethylamino)methyl]benzamide (52 mg, 0.27 mmol) in DMF (5 mL) was added O-(7-azabenzotriazol-1-yl)-1,1,3,3-tetramethyluronium hexafluorophosphate (HATU) (114 mg, 0.3 mmol) and the mixture was stirred for 16 hrs. To the reaction solution were added saturated aqueous solution of sodium hydrogen carbonate (5 mL) and dichloromethane (5 mL), and the organic layer alone was filtered using a PTFE (polytetrafluoroethylene) tube. The filtrate was concentrated and the residue was purified by HPLC (acetonitrile/water=10/90-100/0, containing 0.1% trifluoroacetic acid). The fraction containing the object substance was concentrated and neutralized with saturated aqueous solution of sodium hydrogen carbonate to give the title compound (125 mg, yield 86%) as white crystals.

LC/MS (ESI) m/z 581 (M+H⁺).

The compounds described in the following Examples 184-216 were produced in the similar manner as in Example 183.

### Example 184

### N-[(1R,2S)-1-[({2-(aminocarbonyl)-5-[(dimethylamino)methyl]phenyl}amino)carbonyl]-2-(1H-indol-3-yl)propyl]-4-phenylpiperazine-1-carboxamide

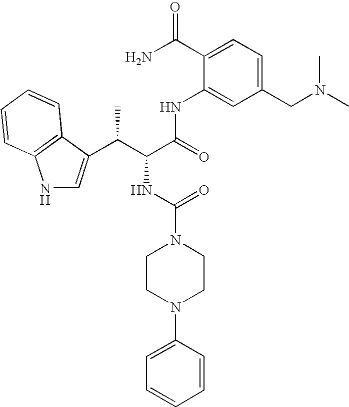

LC/MS (ESI) m/z 582 (M+H⁺).

### Example 185

### N-[(1R,2S)-1-[({2-(aminocarbonyl)-5-[(dimethylamino)methyl]phenyl}amino)carbonyl]-2-(1H-indol-3-yl)propyl]-4-(4-fluorophenyl)piperidine-1-carboxamide

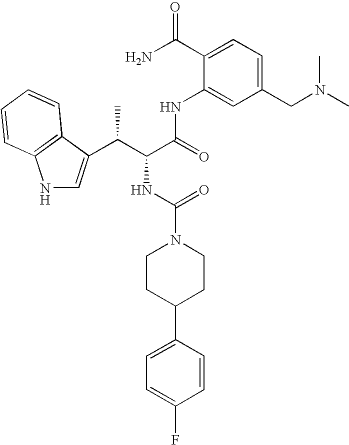

¹H NMR (300 MHz, CDCl₃) δ ppm: 1.40-1.50 (m, 1 H), 1.53 (d, J=7.2 Hz, 3 H), 1.58-1.69 (m, 1 H), 1.70-1.80 (m, 4 H), 2.43 (s, 6 H), 2.54-2.70 (m, 1 H), 2.73-2.98 (m, 2 H), 3.56-3.81 (m, 3 H), 4.03 (dd, J=25.6, 13.8 Hz, 2 H), 4.73 (t, J=6.4 Hz, 1 H), 5.23 (d, J=7.0 Hz, 1 H), 6.92-7.02 (m, 2 H), 7.04 (d, J=7.4 Hz, 1 H), 7.07-7.16 (m, 5 H), 7.30 (d, J=7.9 Hz, 1 H), 7.44 (d, J=8.1 Hz, 1 H), 7.67 (d, J=7.9 Hz, 1 H), 8.41 (s, 1 H), 8.52 (s, 1 H), 11.32 (s, 1 H)

LC/MS (ESI) m/z 599 (M+H⁺).

### Example 186

### N-[(1R,2S)-1-[({2-(aminocarbonyl)-5-[(dimethylamino)methyl]phenyl}amino)carbonyl]-2-(1H-indol-3-yl)propyl]-4-(4-fluorophenyl)piperazine-1-carboxamide

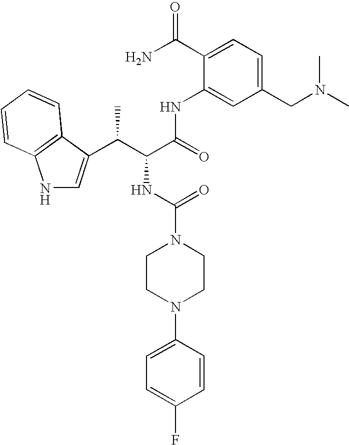

LC/MS (ESI) m/z 600 (M+H⁺).

### Example 187

### N-[(1R,2S)-1-[({2-(aminocarbonyl)-5-[(dimethylamino)methyl]phenyl}amino)carbonyl]-2-(1H-indol-3-yl)propyl]-4-(2-methylphenyl)piperidine-1-carboxamide

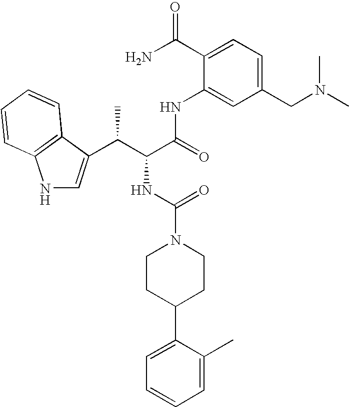

LC/MS (ESI) m/z 595 (M+H⁺)

### Example 188

### N-[(1R,2S)-1-[({2-(aminocarbonyl)-5-[(dimethylamino)methyl]phenyl}amino)carbonyl]-2-(1H-indol-3-yl)propyl]-4-(4-fluoro-2-methylphenyl)piperazine-1-carboxamide

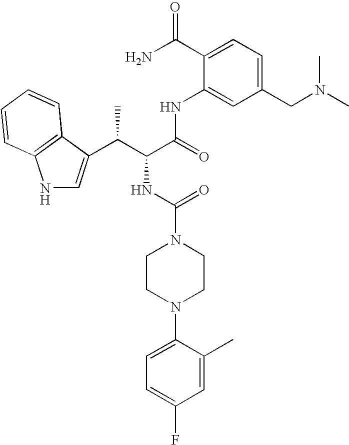

¹H NMR (300 MHz, CDCl₃) δ ppm: 1.55 (d, J=7.2 Hz, 3 H), 2.26 (s, 6 H), 2.28 (s, 3 H), 2.68-2.85 (m, 4 H), 3.36-3.59 (m, 6 H), 3.70-3.82 (m, 1 H), 4.80 (dd, J=7.1, 6.3 Hz, 1 H), 5.25 (d, J=7.4 Hz, 1 H), 6.79-6.94 (m, 3 H), 7.00-7.15 (m, 4 H), 7.30 (d, J=7.9 Hz, 1 H), 7.40 (d, J=8.1 Hz, 1 H), 7.68 (d, J=7.9 Hz, 1 H), 8.32 (s, 1 H), 8.43 (d, J=1.3 Hz, 1 H), 11. 32 (s, 1 H).

LC/MS (ESI) m/z 614 (M+H⁺).

### Example 189

### N-[(1R,2S)-1-[({5-[(dimethylamino)methyl]-2-[(methylamino)carbonyl]phenyl}amino)carbonyl]-2-(1H-indol-3-yl)propyl]-4-phenylpiperidine-1-carboxamide

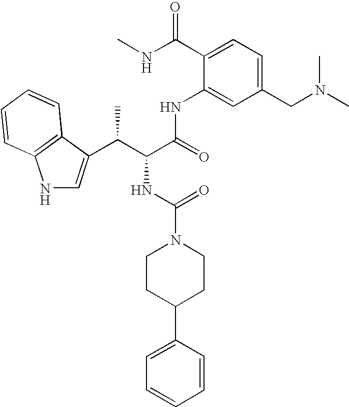

LC/MS (ESI) m/z 595 (M+H⁺).

### Example 190

### N-[(1R,2S)-1-[({5-[(dimethylamino)methyl]-2-[(methylamino)carbonyl]phenyl)amino)carbonyl]-2-(1H-indol-3-yl)propyl]-4-phenylpiperazine-1-carboxamide

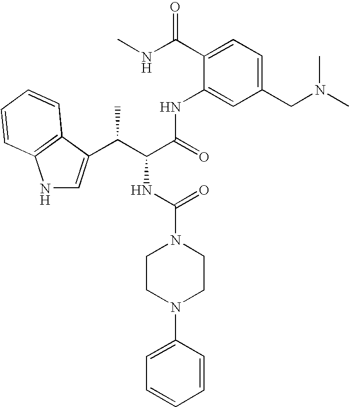

LC/MS (ESI) m/z 596 (M+H⁺).

### Example 191

### N-[(1R,2S)-1-[({5-[(dimethylamino)methyl]-2-[(methylamino)carbonyl]phenyl}amino)carbonyl]-2-(1H-indol-3-yl)propyl]-4-(4-fluorophenyl)piperidine-1-carboxamide

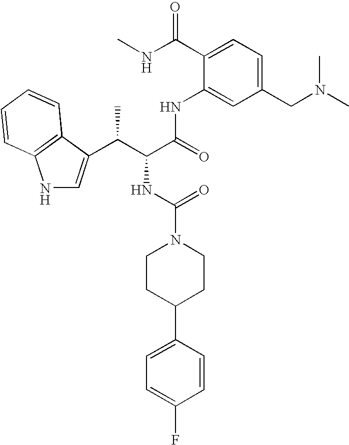

LC/MS (ESI) m/z 613 (M+H⁺).

### Example 192

### N-[(1R,2S)-1-[({5-[(dimethylamino)methyl]-2-[(methylamino)carbonyl]phenyl}amino)carbonyl]-2-(1H-indol-3-yl)propyl]-4-(4-fluorophenyl)piperazine-1-carboxamide

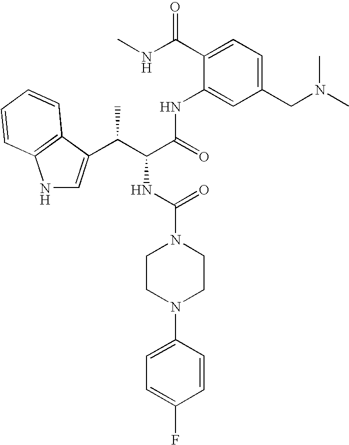

LC/MS (ESI) m/z 614 (M+H⁺).

### Example 193

### N-[(1R,2S)-1-[({5-[(dimethylamino)methyl]-2-[(methylamino)carbonyl]phenyl}amino)carbonyl]-2-(1H-indol-3-yl)propyl]-4-(2-methylphenyl)piperidine-1-carboxamide

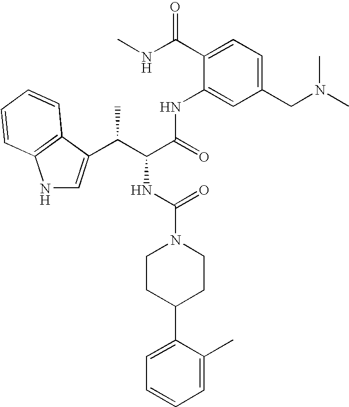

LC/MS (ESI) m/z 609 (M+H⁺).

### Example 194

### N-[(1R,2S)-1-[({5-[(dimethylamino)methyl]-2-[(methylamino)carbonyl]phenyl}amino)carbonyl]-2-(1H-indol-3-yl)propyl]-4-(4-fluoro-2-methylphenyl)piperazine-1-carboxamide

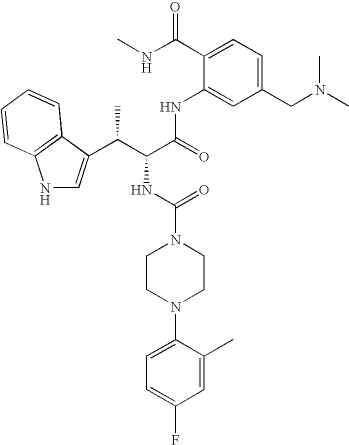

LC/MS (ESI) m/z 628 (M+H⁺).

### Example 195

### N-[(1R,2S)-1-[({5-[(dimethylamino)methyl]-2-[(ethylamino)carbonyl]phenyl}amino)carbonyl]-2-(1H-indol-3-yl)propyl]-4-phenylpiperidine-1-carboxamide

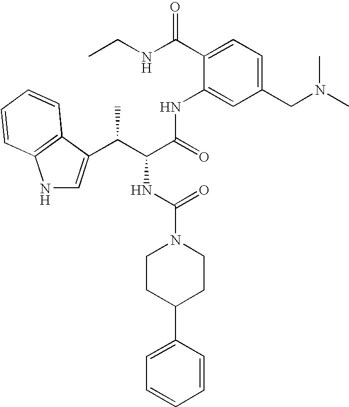

LC/MS (ESI) m/z 609 (M+H⁺).

### Example 196

### N-[(1R,2S)-1-[({5-[(dimethylamino)methyl]-2-[(ethylamino)carbonyl]phenyl}amino)carbonyl]-2-(1H-indol-3-yl)propyl]-4-phenylpiperazine-1-carboxamide

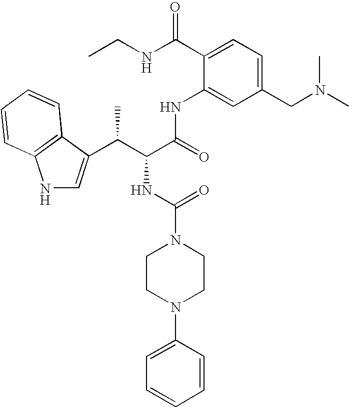

LC/MS (ESI) m/z 610 (M+H⁺).

### Example 197

### N-[(1R,2S)-1-[({5-[(dimethylamino)methyl]-2-[(ethylamino)carbonyl]phenyl}amino)carbonyl]-2-(1H-indol-3-yl)propyl]-4-(4-fluorophenyl)piperidine-1-carboxamide

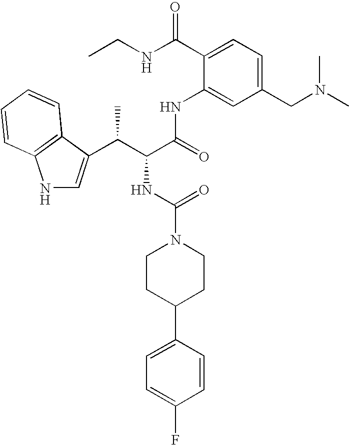

LC/MS (ESI) m/z 627 (M+H⁺).

### Example 198

### N-[(1R,2S)-1-[({5-[(dimethylamino)methyl]-2-[(ethylamino)carbonyl]phenyl}amino)carbonyl]-2-(1H-indol-3-yl)propyl]-4-(4-fluorophenyl)piperazine-1-carboxamide

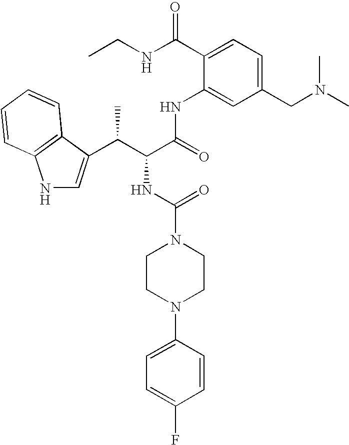

LC/MS (ESI) m/z 628 (M+H⁺).

### Example 199

### N-[(1R,2S)-1-[({5-[(dimethylamino)methyl]-2-[(ethylamino)carbonyl]phenyl}amino)carbonyl]-2-(1H-indol-3-yl)propyl]-4-(2-methylphenyl)piperidine-1-carboxamide

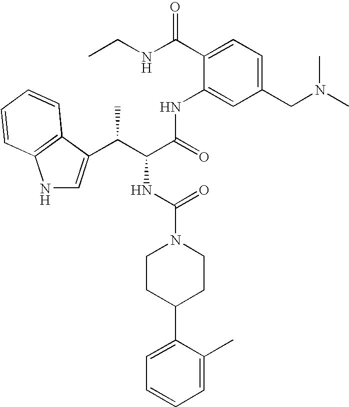

LC/MS (ESI) m/z 623 (M+H⁺).

### Example 200

### N-[(1R,2S)-1-[({5-[(dimethylamino)methyl]-2-[(ethylamino)carbonyl]phenyl}amino)carbonyl]-2-(1H-indol-3-yl)propyl]-4-(4-fluoro-2-methylphenyl)piperazine-1-carboxamide

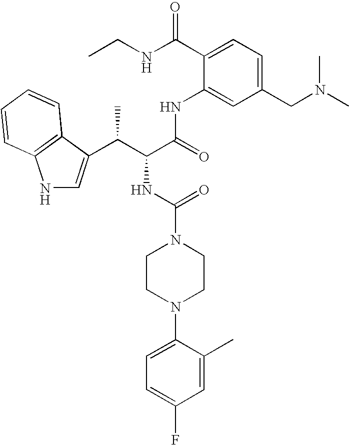

LC/MS (ESI) m/z 642 (M+H⁺).

### Example 201

### N-[(1R,2S)-1-[({2-[(dimethylamino)carbonyl]-5-[(dimethylamino)methyl]phenyl}amino)carbonyl]-2-(1H-indol-3-yl)propyl]-4-phenylpiperidine-1-carboxamide

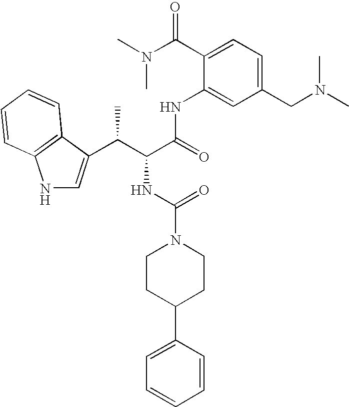

LC/MS (ESI) m/z 609 (M+H⁺).

### Example 202

### N-[(1R,2S)-1-[({2-[(dimethylamino)carbonyl]-5-[(dimethylamino)methyl]phenyl}amino)carbonyl]-2-(1H-indol-3-yl)propyl]-4-phenylpiperazine-1-carboxamide

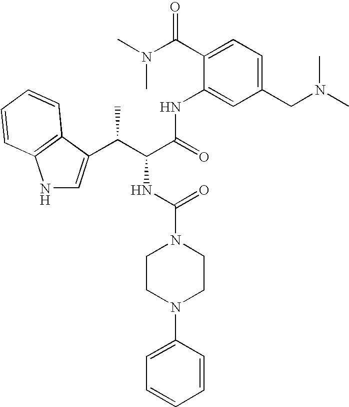

LC/MS (ESI) m/z 610 (M+H⁺).

### Example 203

### N-[(1R,2S)-1-[({2-[(dimethylamino)carbonyl]-5-[(dimethylamino)methyl]phenyl}amino)carbonyl]-2-(1H-indol-3-yl)propyl]-4-(4-fluorophenyl)piperidine-1-carboxamide

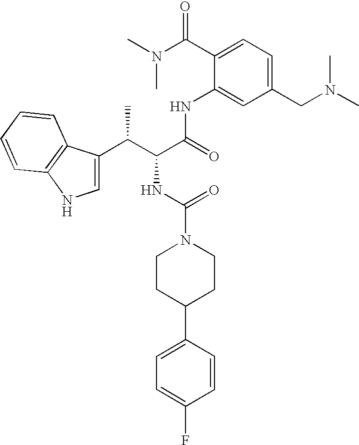

¹H NMR (300 MHz, CDCl₃) δ ppm: 1.34-1.48 (m, 1 H), 1.55 (d, J=7.4 Hz, 3 H), 1.75 (s, 2 H), 2.24 (s, 6 H), 2.53-2.68 (m, 1 H), 2.72-3.04 (m, 8 H), 3.33-3.45 (m, 2 H), 3.73-3.85 (m, 1 H), 3.90-4.14 (m, 2 H), 4.78 (dd, J=7.1, 5.9 Hz, 1 H), 5.15 (d, J=7.2 Hz, 1 H), 6.93-7.02 (m, 2 H), 7.03-7.20 (m, 8 H), 7.28-7.36 (m, 1 H), 7.72 (d, J=7.2 Hz, 1 H), 8.01 (s, 1 H), 8.10 (s, 1 H), 9.18 (s. 1 H).

LC/MS (ESI) m/z 627 (M+H⁺).

### Example 204

### N-[(1R,2S)-1-[({2-[(dimethylamino)carbonyl]-5-[(dimethylamino)methyl]phenyl}amino)carbonyl]-2-(1H-indol-3-yl)propyl]-4-(4-fluorophenyl)piperazine-1-carboxamide

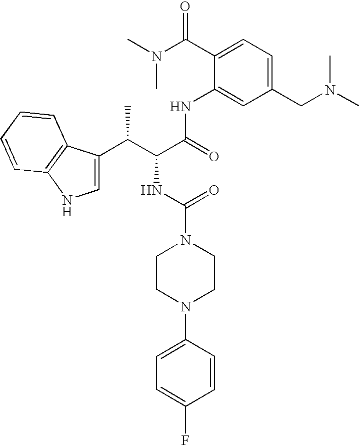

LC/MS (ESI) m/z 628 (M+H⁺)

### Example 205

### N-[(1R,2S)-1-[({2-[(dimethylamino)carbonyl]-5-[(dimethylamino)methyl]phenyl}amino)carbonyl]-2-(1H-indol-3-yl)propyl]-4-(2-methylphenyl)piperidine-1-carboxamide

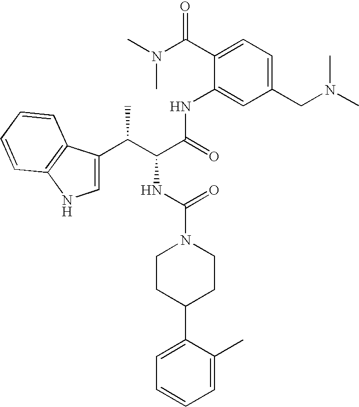

LC/MS (ESI) m/z 623 (M+H⁺).

### Example 206

### N-[(1R,2S)-1-[({2-[(dimethylamino)carbonyl]-5-[(dimethylamino)methyl]phenyl}amino)carbonyl]-2-(1H-indol-3-yl)propyl]-4-(4-fluoro-2-methylphenyl)piperazine-1-carboxamide

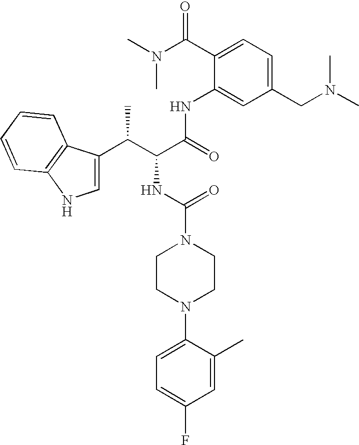

LC/MS (ESI) m/z 642 (M+H⁺).

### Example 207

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-phenylpiperidine-1-carboxamide

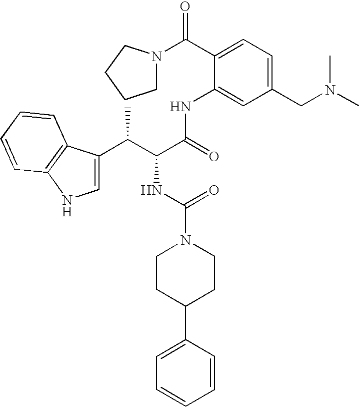

LC/MS (ESI) m/z 634 (M+H⁺).

### Example 208

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-phenylpiperazine-1-carboxamide

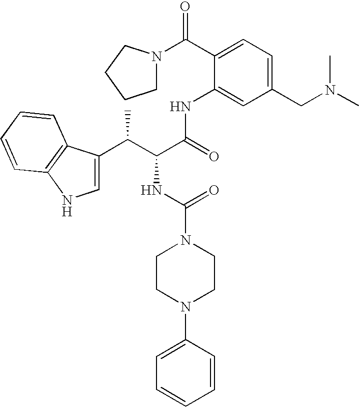

LC/MS (ESI) m/z 635 (M+H⁺).

### Example 209

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-(4-fluorophenyl)piperidine-1-carboxamide

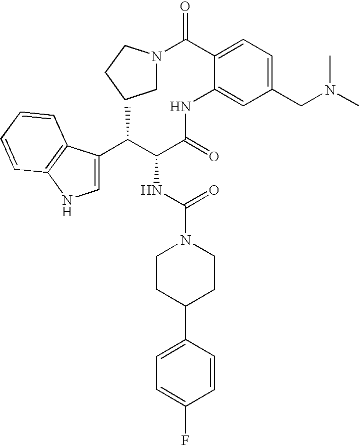

LC/MS (ESI) m/z 652 (M+H⁺).

### Example 210

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-(4-fluorophenyl)piperazine-1-carboxamide

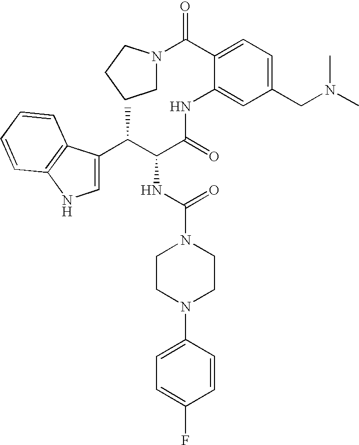

LC/MS (ESI) m/z 653 (M+H⁺).

### Example 211

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-(2-methylphenyl)piperidine-1-carboxamide

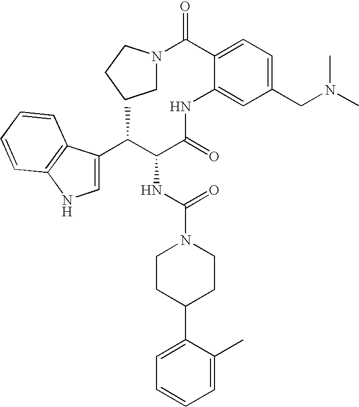

LC/MS (ESI) m/z 649 (M+H⁺).

### Example 212

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-(2-methylphenyl)piperazine-1-carboxamide

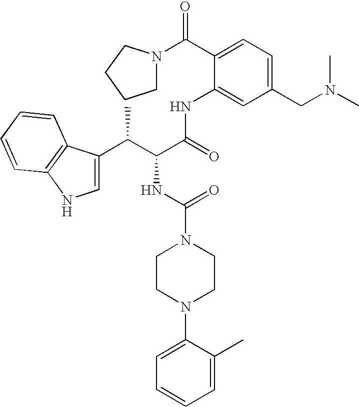

LC/MS (ESI) m/z 650 (M+H⁺).

### Example 213

### N-[(1R,2S)-1-({[5-[(dimethylamino)methyl]-2-(pyrrolidin-1-ylcarbonyl)phenyl]amino}carbonyl)-2-(1H-indol-3-yl)propyl]-4-(4-fluoro-2-methylphenyl)piperazine-1-carboxamide

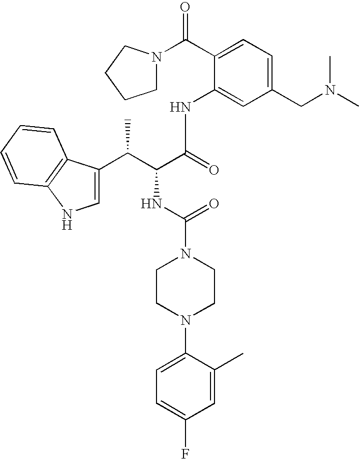

LC/MS (ESI) m/z 668 (M+H⁺).

### Example 214

### methyl 4-[(dimethylamino)methyl]-2-[((2R,3S)-3-(1H-indol-3-yl)-2-{[(4-phenylpiperidin-1-yl)carbonyl]amino}butanoyl)amino]benzoate

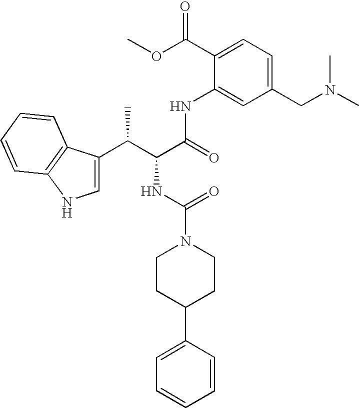

LC/MS (ESI) m/z 596 (M+H⁺).

### Example 215

### Methyl 4-[(dimethylamino)methyl]-2-{[(2R,3S)-2-({[4-(4-fluorophenyl)piperazin-1-yl]carbonyl}amino)-3-(1H-indol-3-yl)butanoyl]amino}benzoate

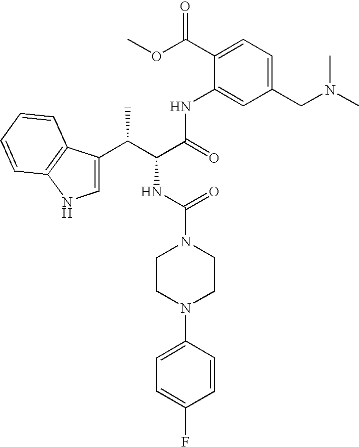

LC/MS (ESI) m/z 615 (M+H⁺).

### Example 216

### Methyl 4-[(dimethylamino)methyl]-2-{[(2R,3S)-3-(1H-indol-3-yl)-2-({[4-(2-methylphenyl)piperidin-1-yl]carbonyl}amino)butanoyl]amino}benzoate

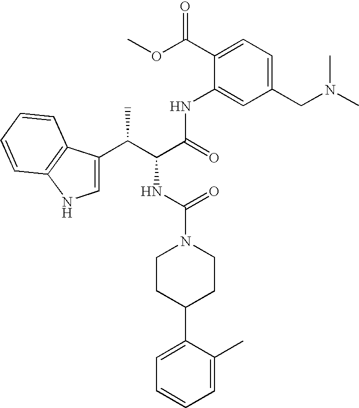

LC/MS (ESI) m/z 610 (M+H⁺).

### Example 217

### 4-[(dimethylamino)methyl]-2-[((2R,3S)-3-(1H-indol-3-yl)-2-{[(4-phenylpiperidin-1-yl)carbonyl]amino}butanoyl)amino]benzoic acid

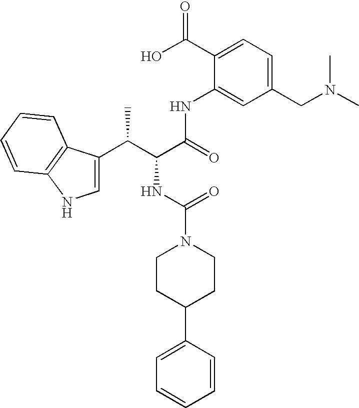

To a solution of methyl 4-[(dimethylamino)methyl]-2-[((2R,3S)-3-(1H-indol-3-yl)-2-{[(4-phenylpiperidin-1-yl)carbonyl]amino}butanoyl)amino]benzoate (150 mg, 0.25 mmol) in THF (1 mL)-methanol (1 mL) was added 1N aqueous sodium hydroxide solution (1 mL) and the mixture was stirred at room temperature for 8 hrs. 1N Hydrochloric acid was poured into the reaction solution to acidify the solution, which was then extracted with ethyl acetate. The extract was washed with saturated brine and dried (MgSO₄). The solvent was evaporated under reduced pressure and the residue was crystallized from diisopropyl ether to give the title compound (143 mg, yield 96%) as white crystals.

LC/MS (ESI) m/z 582 (M+H⁺).

The compounds described in the following Examples 218-258 were produced in the similar manner as in Example 183.

### Example 218

### N-[(1R,2S)-1-({[5-[(dimethylamino)methyl]-2-(2-thienyl)phenyl]amino}carbonyl)-2-(1H-indol-3-yl)propyl]-4-phenylpiperidine-1-carboxamide

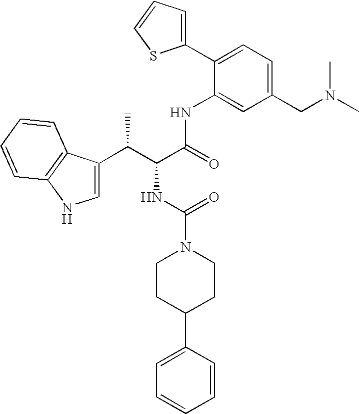

LC/MS (ESI) m/z 620 (M+H⁺).

### Example 219

### N-[(1R,2S)-1-({[5-[(dimethylamino)methyl]-2-(2-thienyl)phenyl]amino}carbonyl)-2-(1H-indol-3-yl)propyl]-4-phenylpiperazine-1-carboxamide

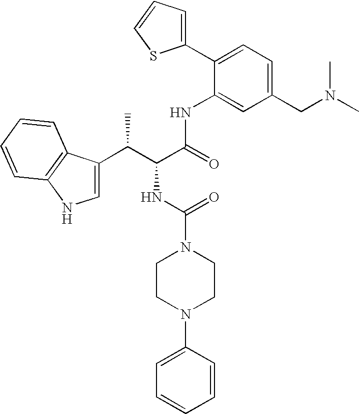

LC/MS (ESI) m/z 621 (M+H⁺)

### Example 220

### N-[(1R,2S)-1-({[5-[(dimethylamino)methyl]-2-(2-thienyl)phenyl]amino}carbonyl)-2- (1H-indol-3-yl)propyl]-4-(4-fluorophenyl)piperidine-1-carboxamide

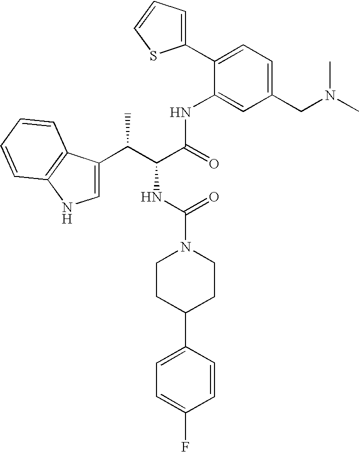

LC/MS (ESI) m/z 638 (M+H⁺).

### Example 221

### N-[(1R,2S)-1-({[5-[(dimethylamino)methyl]-2-(2-thienyl)phenyl]amino}carbonyl)-2-(1H-indol-3-yl)propyl]-4-(2-methylphenyl)piperidine-1-carboxamide

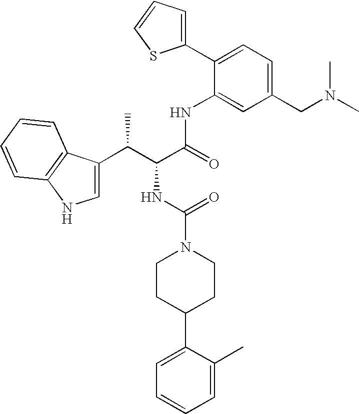

LC/MS (ESI) m/z 634 (M+H⁺).

### Example 222

### N-[(1R,2S)-1-({[5-[(dimethylamino)methyl]-2-(3-thienyl)phenyl]amino}carbonyl)-2-(1H-indol-3-yl)propyl]-4-phenylpiperidine-1-carboxamide

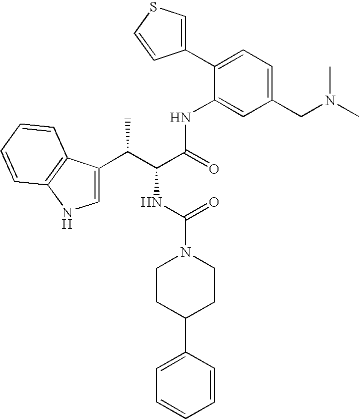

LC/MS (ESI) m/z 620 (M+H⁺).

### Example 223

### N-[(1R,2S)-1-({[5-[(dimethylamino)methyl]-2-(3-thienyl)phenyl]amino}carbonyl)-2-(1H-indol-3-yl)propyl]-4-phenylpiperazine-1-carboxamide

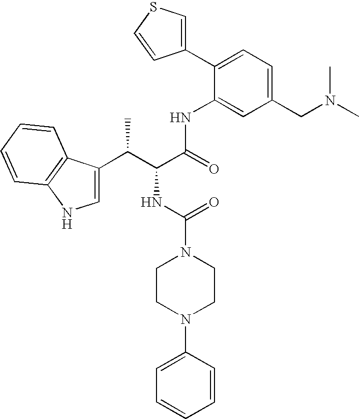

LC/MS (ESI) m/z 621 (M+H⁺).

### Example 224

### N-[(1R,2S)-1-({[5-[(dimethylamino)methyl]-2-(3-thienyl)phenyl]amino}carbonyl)-2-(1H-indol-3-yl)propyl]-4-(4-fluorophenyl)piperidine-1-carboxamide

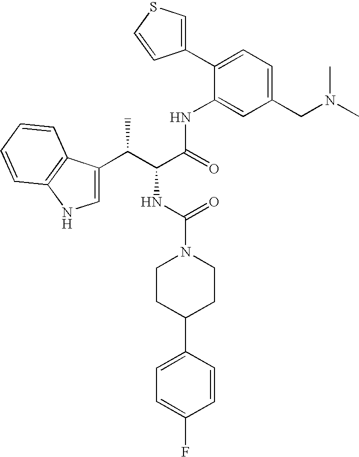

LC/MS (ESI) m/z 638 (M+H⁺)

### Example 225

### N-[(1R,2S)-1-({[5-[(dimethylamino)methyl]-2-(3-thienyl)phenyl]amino}carbonyl)-2-(1H-indol-3-yl)propyl]-4-(4-fluorophenyl)piperazine-1-carboxamide

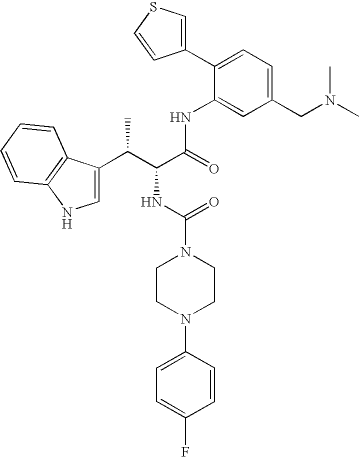

LC/MS (ESI) m/z 639 (M+H⁺).

### Example 226

### N-[(1R,2S)-1-({[5-[(dimethylamino)methyl]-2-(3-thienyl)phenyl]amino}carbonyl)-2-(1H-indol-3-yl)propyl]-4-(2-methylphenyl)piperidine-1-carboxamide

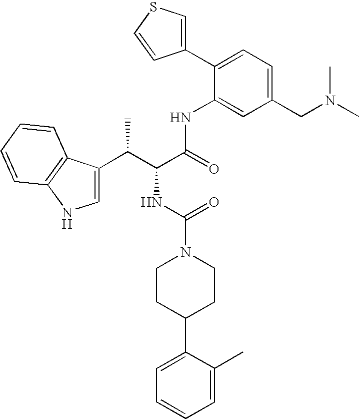

LC/MS (ESI) m/z 634 (M+H⁺).

### Example 227

### N-[(1R,2S)-1-({[5-[(dimethylamino)methyl]-2-(1-methyl-1H-pyrazol-4-yl)phenyl]amino}carbonyl)-2-(1H-indol-3-yl)propyl]-4-phenylpiperidine-1-carboxamide

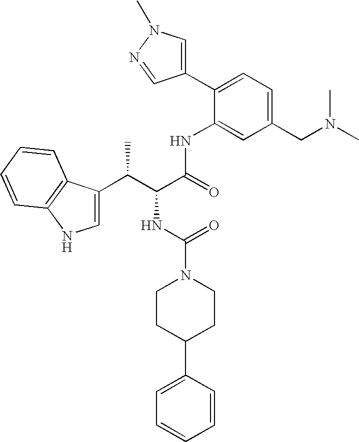

LC/MS (ESI) m/z 618 (M+H⁺).

### Example 228

### N-[(1R,2S)-1-({[5-[(dimethylamino)methyl]-2-(1-methyl-1H-pyrazol-4-yl)phenyl]amino}carbonyl)-2-(1H-indol-3-yl)propyl]-4-phenylpiperazine-1-carboxamide

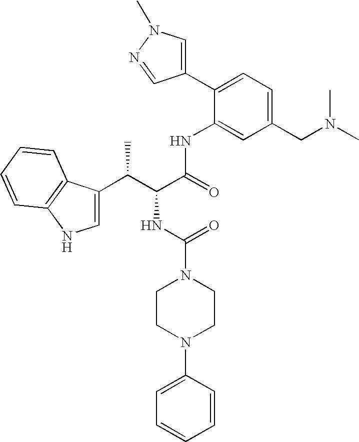

LC/MS (ESI) m/z 619 (M+H⁺).

### Example 229

### N-[(1R,2S)-1-({[5-[(dimethylamino)methyl]-2-(1-methyl-1H-pyrazol-4-yl)phenyl]amino}carbonyl)-2-(1H-indol-3-yl)propyl]-4-(4-fluorophenyl)piperidine-1-carboxamide

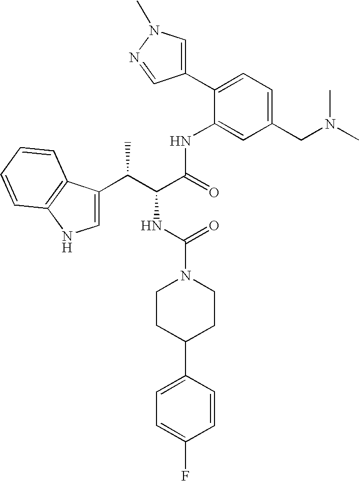

LC/MS (ESI) m/z 636 (M+H⁺).

### Example 230

### N-[(1R,2S)-1-({[5-[(dimethylamino)methyl]-2-(1-methyl-1H-pyrazol-4-yl)phenyl]amino}carbonyl)-2-(1H-indol-3-yl)propyl]-4-(2-methylphenyl)piperidine-1-carboxamide

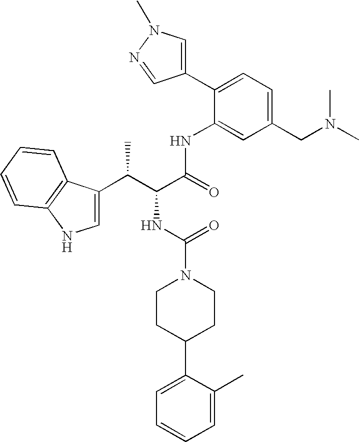

LC/MS (ESI) m/z 632 (M+H⁺).

### Example 231

### N-[(1R,2S)-1-({[5-[(dimethylamino)methyl]-2-(2-furyl)phenyl]amino}carbonyl)-2-(1H-indol-3-yl)propyl]-4-phenylpiperidine-1-carboxamide

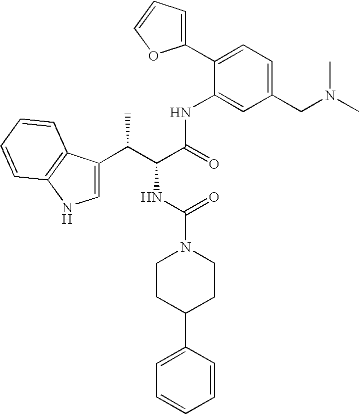

LC/MS (ESI) m/z 604 (M+H⁺).

### Example 232

### N-[(1R,2S)-1-({[5-[(dimethylamino)methyl]-2-(2-furyl)phenyl]amino}carbonyl)-2-(1H-indol-3-yl)propyl]-4-(4-fluorophenyl)piperidine-1-carboxamide

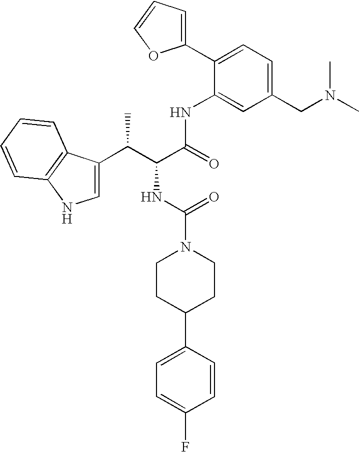

LC/MS (ESI) m/z 623 (M+H⁺).

### Example 233

### N-[(1R,2S)-1-({[5-[(dimethylamino)methyl]-2-(methylthio)phenyl]amino}carbonyl)-2-(1H-indol-3-yl)propyl]-4-phenylpiperidine-1-carboxamide

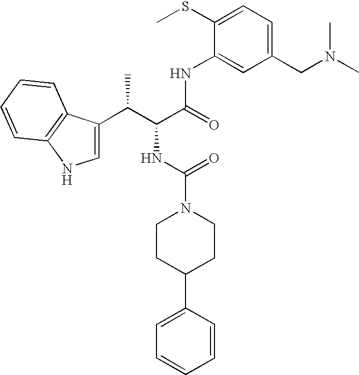

LC/MS (ESI) m/z 584 (M+H⁺).

### Example 234

### N-[(1R,2S)-1-({[5-[(dimethylamino)methyl]-2-(methylthio)phenyl]amino}carbonyl)-2-(1H-indol-3-yl)propyl]-4-phenylpiperazine-1-carboxamide

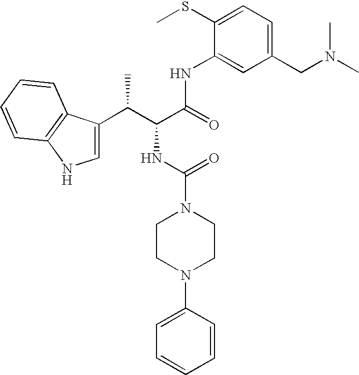

LC/MS (ESI) m/z 585 (M+H⁺).

### Example 235

### N-[(1R,2S)-1-({[5-[(dimethylamino)methyl]-2-(methylthio)phenyl]amino}carbonyl)-2-(1H-indol-3-yl)propyl]-4-(4-fluorophenyl)piperidine-1-carboxamide

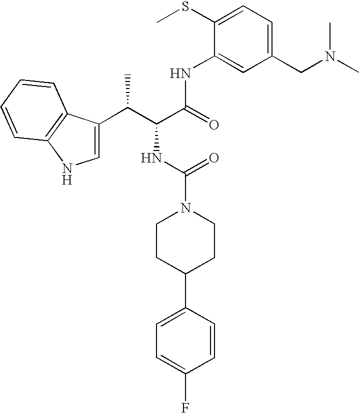

LC/MS (ESI) m/z 602 (M+H⁺).

### Example 236

### N-[(1R,2S)-1-({[5-[(dimethylamino)methyl]-2-(methylthio)phenyl]amino}carbonyl)-2-(1H-indol-3-yl)propyl]-4-(4-fluorophenyl)piperazine-1-carboxamide

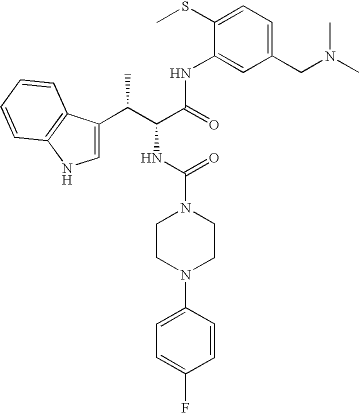

LC/MS (ESI) m/z 603 (M+H⁺).

### Example 237

### N-[(1R,2S)-1-({[5-[(dimethylamino)methyl]-2-(methylthio)phenyl]amino}carbonyl)-2-(1H-indol-3-yl)propyl]-4-(2-methylphenyl)piperidine-1-carboxamide

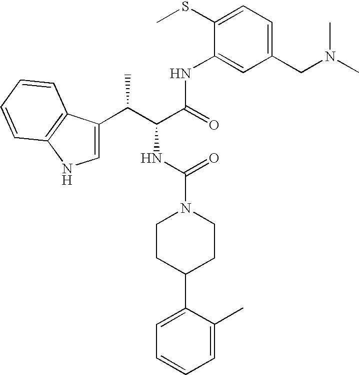

LC/MS (ESI) m/z 598 (M+H⁺).

### Example 238

### N-[(1R,2S)-1-({[5-[(dimethylamino)methyl]-2-(methylthio)phenyl]amino}carbonyl)-2-(1H-indol-3-yl)propyl]-4-(4-fluoro-2-methylphenyl)piperazine-1-carboxamide

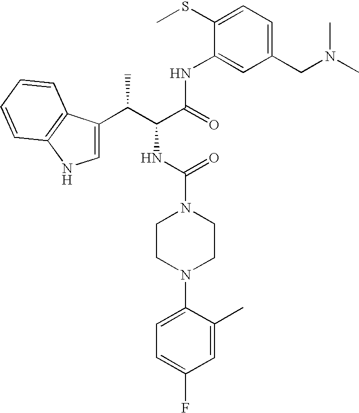

LC/MS (ESI) m/z 617 (M+H⁺).

### Example 239

### N-[(1R,2S)-1-({[5-[(dimethylamino)methyl]-2-(methylsulfinyl)phenyl]amino}carbonyl)-2-(1H-indol-3-yl)propyl]-4-phenylpiperidine-1-carboxamide

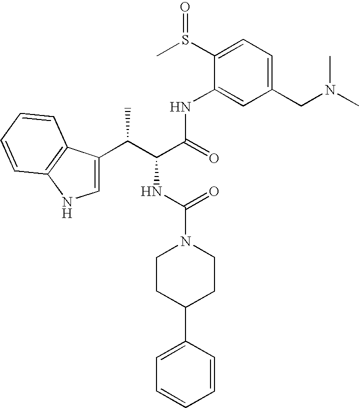

LC/MS (ESI) m/z 600 (M+H⁺).

### Example 240

### N-[(1R,2S)-1-({[5-[(dimethylamino)methyl]-2-(methylsulfinyl)phenyl]amino}carbonyl)-2-(1H-indol-3-yl)propyl]-4-(2-methylphenyl)piperazine-1-carboxamide

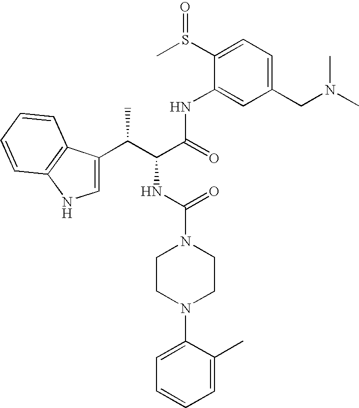

LC/MS (ESI) m/z 615 (M+H⁺).

### Example 241

### N-[(1R,2S)-1-({[5-[(dimethylamino)methyl]-2-(methylsulfinyl)phenyl]amino}carbonyl)-2-(1H-indol-3-yl)propyl]-4-(4-fluoro-2-methylphenyl)piperazine-1-carboxamide

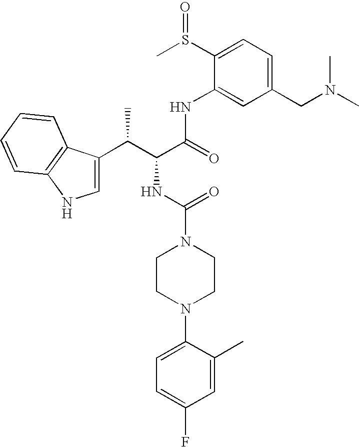

LC/MS (ESI) m/z 633 (M+H⁺).

### Example 242

### N-[(1R,2S)-1-({[5-[(dimethylamino)methyl]-2-(methylsulfonyl)phenyl]amino}carbonyl)-2-(1H-indol-3-yl)propyl]-4-phenylpiperidine-1-carboxamide

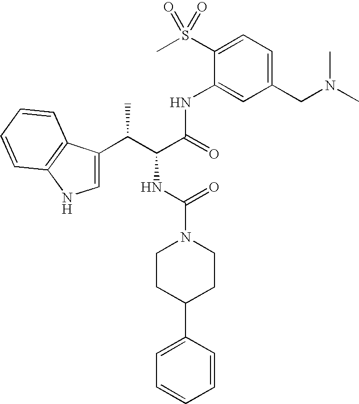

LC/MS (ESI) m/z 616 (M+H⁺).

### Example 243

### N-[(1R,2S)-1-({[5-[(dimethylamino)methyl]-2-(methylsulfonyl)phenyl]amino}carbonyl)-2-(1H-indol-3-yl)propyl]-4-phenylpiperazine-1-carboxamide

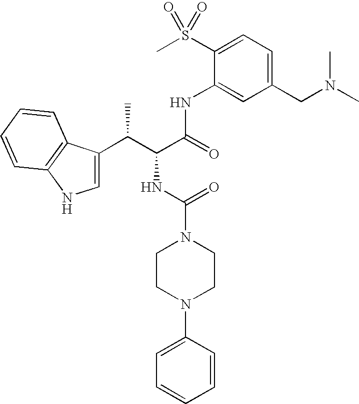

LC/MS (ESI) m/z 617 (M+H⁺).

### Example 244

### N-[(1R,2S)-1-({[5-[(dimethylamino)methyl]-2-(methylsulfonyl)phenyl]amino}carbonyl)-2-(1H-indol-3-yl)propyl]-4-(4-fluorophenyl)piperidine-1-carboxamide

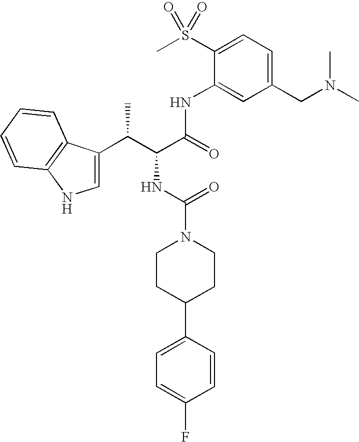

LC/MS (ESI) m/z 634 (M+H⁺).

### Example 245

### N-[(1R,2S)-1-({[5-[(dimethylamino)methyl]-2-(methylsulfonyl)phenyl]amino}carbonyl)-2-(1H-indol-3-yl)propyl]-4-(4-fluorophenyl)piperazine-1-carboxamide

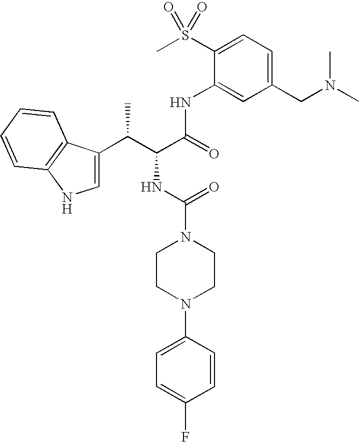

LC/MS (ESI) m/z 635 (M+H⁺).

### Example 246

### N-[(1R,2S)-1-(([5-[(dimethylamino)methyl]-2-(methylsulfonyl)phenyl]amino}carbonyl)-2-(1H-indol-3-yl)propyl]-4-(2-methylphenyl)piperidine-1-carboxamide

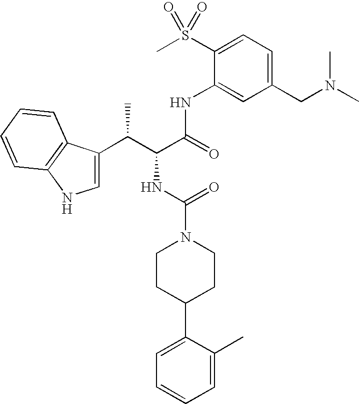

LC/MS (ESI) m/z 630 (M+H⁺).

### Example 247

### N-[(1R,2S)-1-({[5-[(dimethylamino)methyl]-2-(methylsulfonyl)phenyl]amino}carbonyl)-2-(1H-indol-3-yl)propyl]-4-(2-methylphenyl)piperazine-1-carboxamide

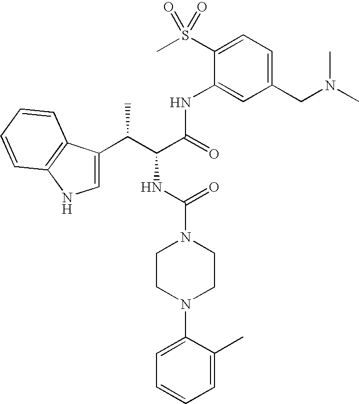

LC/MS (ESI) m/z 631 (M+H⁺).

### Example 248

### N-[(1R,2S)-1-({[5-[(dimethylamino)methyl]-2-(methylsulfonyl)phenyl]amino}carbonyl)-2-(1H-indol-3-yl)propyl]-4-(4-fluoro-2-methylphenyl)piperazine-1-carboxamide

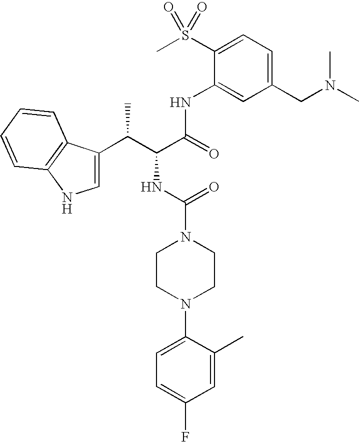

LC/MS (ESI) m/z 649 (M+H⁺).

### Example 249

### N-[(1R,2S)-1-(([5-[(dimethylamino)methyl]-2-(isopropylthio)phenyl]amino}carbonyl)-2-(1H-indol-3-yl)propyl]-4-(4-fluorophenyl)piperazine-1-carboxamide

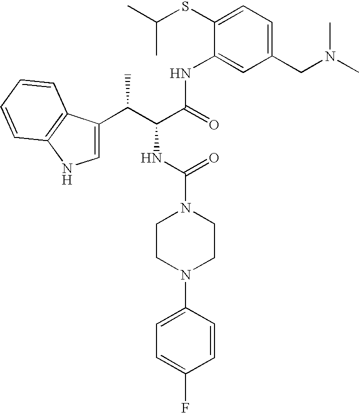

LC/MS (ESI) m/z 631 (M+H⁺).

### Example 250

### N-[(1R,2S)-1-(([5-[(dimethylamino)methyl]-2-(isopropylthio)phenyl]amino}carbonyl)-2-(1H-indol-3-yl)propyl]-4-(4-fluoro-2-methylphenyl)piperazine-1-carboxamide

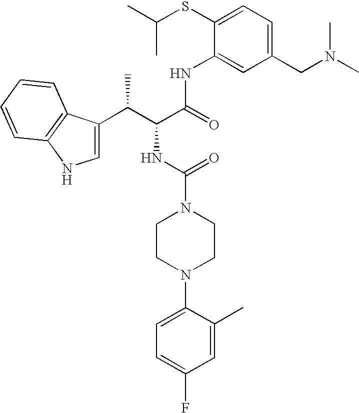

LC/MS (ESI) m/z 645 (M+H⁺).

### Example 251

### N-[(1R, 2S) -1-[({3-[-(dimethylamino)ethyl]phenyl}amino)carbonyl]-2-(1H-indol-3-yl)propyl]-4-phenylpiperidine-1-carboxamide

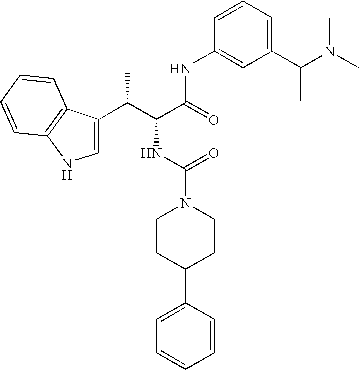

LC/MS (ESI) m/z 552 (M+H⁺).

### Example 252

### N-[(1R,2S)-1-[({4-[(dimethylamino)methyl]-1,3-thiazol-2-yl}amino)carbonyl]-2-(1H-indol-3-yl)propyl]-4-phenylpiperidine-1-carboxamide

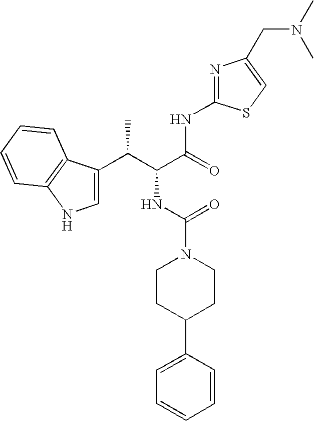

LC/MS (ESI) m/z 545 (M+H⁺).

### Example 253

### N-[(1R,2S)-1-[({4-[(dimethylamino)methyl]-1,3-thiazol-2-yl}amino)carbonyl]-2-(1H-indol-3-yl)propyl]-4-phenylpiperazine-1-carboxamide

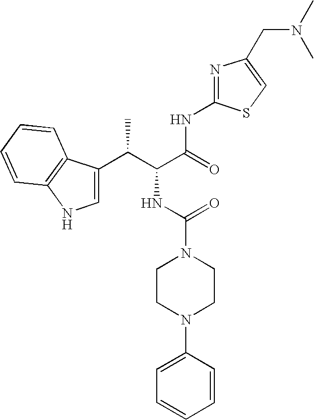

LC/MS (ESI) m/z 546 (M+H⁺).

### Example 254

### N-[(1R,2S)-1-[({4-[(dimethylamino)methyl]-1,3-thiazol-2-yl}amino)carbonyl]-2-(1H-indol-3-yl)propyl]-4-(4-fluorophenyl)piperidine-1-carboxamide

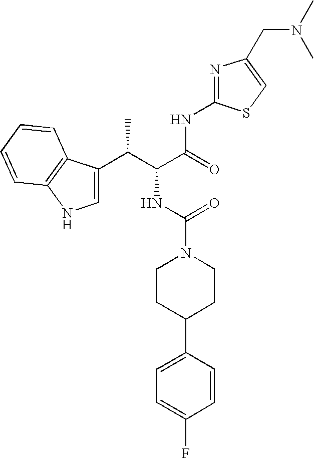

LC/MS (ESI) m/z 563 (M+H⁺).

### Example 255

### N-[(1R,2S)-1-[({4-[(dimethylamino)methyl]-1,3-thiazol-2-yl}amino)carbonyl]-2-(1H-indol-3-yl)propyl]-4-(4-fluorophenyl)piperazine-1-carboxamide

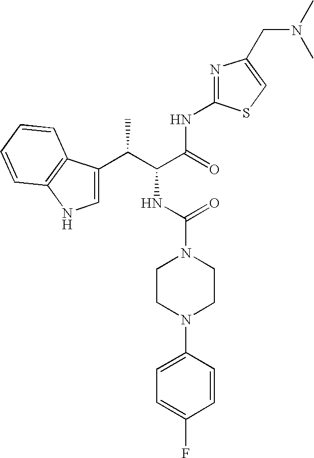

LC/MS (ESI) m/z 564 (M+H⁺).

### Example 256

### N-[(1R,2S)-1-[({4-[(dimethylamino)methyl]-1,3-thiazol-2-yl}amino)carbonyl]-2-(1H-indol-3-yl)propyl]-4-(2-methylphenyl)piperidine-1-carboxamide

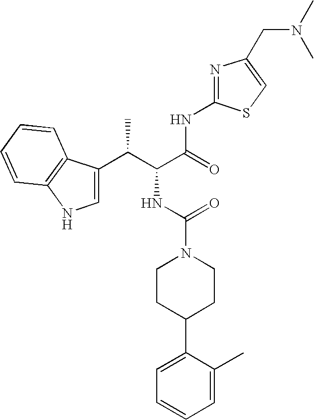

LC/MS (ESI) m/z 559 (M+H⁺).

### Example 257

### N-[(1R,2S)-1-[({4-[(dimethylamino)methyl]-1,3-thiazol-2-yl}amino)carbonyl]-2-(1H-indol-3-yl)propyl]-4-(2-methylphenyl)piperazine-1-carboxamide

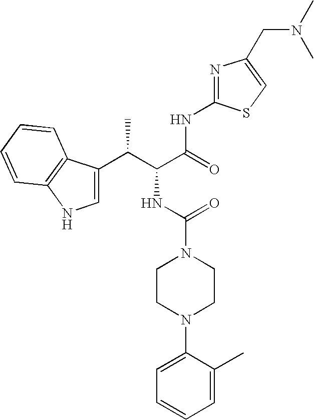

LC/MS (ESI) m/z 560 (M+H⁺).

### Example 258

### N-[(1R,2S)-1-[({4-[(dimethylamino)methyl]-1,3-thiazol-2-yl}amino)carbonyl]-2-(1H-indol-3-yl)propyl]-4-(4-fluoro-2-methylphenyl)piperazine-1-carboxamide

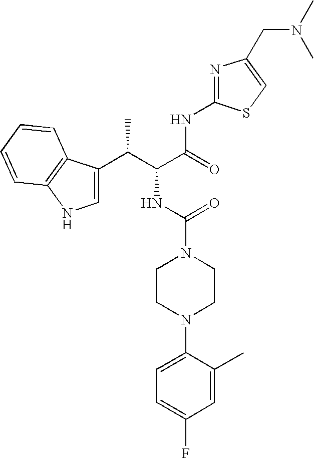

LC/MS (ESI) m/z 578 (M+H⁺).

The compounds described in the following Examples 259-272 were produced in the similar manner as in Example 1.

### Example 259

### N-[(1R,2S)-1-[((2-(cyclopropylmethoxy)-5-[(dimethylamino)methyl]phenyl}amino)carbonyl]-2-(1H-indol-3-yl)propyl]-4-(4-fluorophenyl)piperazine-1-carboxamide

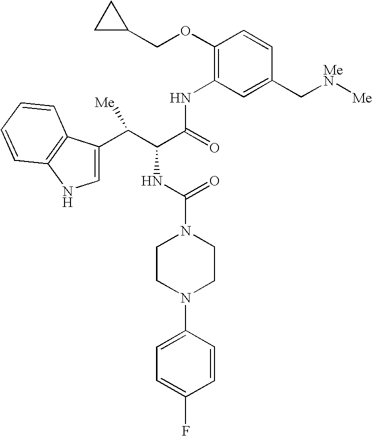

LC/MS (ESI) m/z 627 (M+H⁺).

### Example 260

### N-[(1R,2S)-1-[({2-(cyclopropylmethoxy)-5-[(dimethylamino)methyl]phenyl}amino)carbonyl]-2-(1H-indol-3-yl)propyl]-4-(4-fluoro-2-methylphenyl)piperazine-1-carboxamide

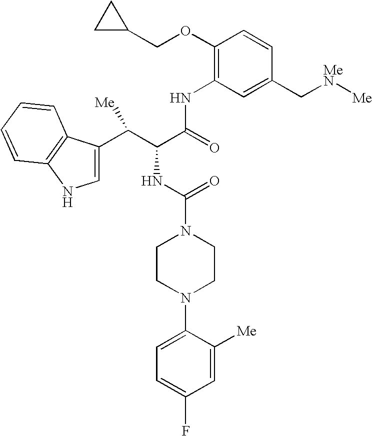

LC/MS (ESI) m/z 641 (M+H⁺).

### Example 261

### N-[(1R,2S)-1-[({2-(cyclopropylmethoxy)-5-[(dimethylamino)methyl]phenyl}amino)carbonyl]-2-(1H-indol-3-yl)propyl]-4-(4-methylphenyl)piperazine-1-carboxamide

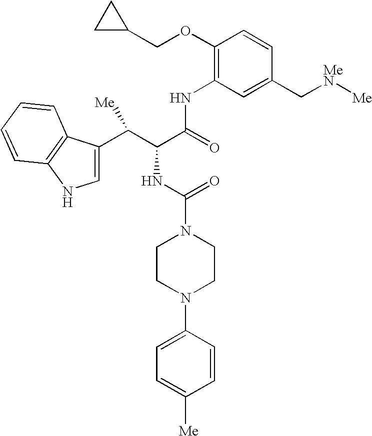

LC/MS (ESI) m/z 623 (M+H⁺).

### Example 262

### 4-(4-chlorophenyl)-N-[(1R,2S)-1-[({2-(cyclopropylmethoxy)-5-[(dimethylamino)methyl]phenyl}amino)carbonyl]-2-(1H-indol-3-yl)propyl]piperazine-1-carboxamide

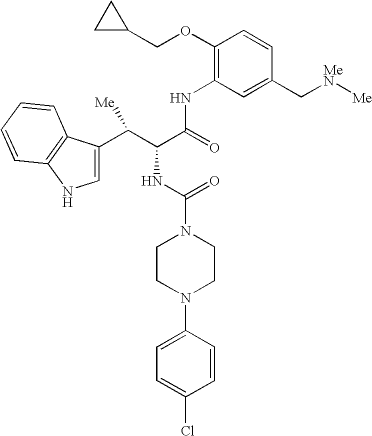

LC/MS (ESI) m/z 643 (M+H⁺).

### Example 263

### N-[(1R,2S)-1-[({2-(cyclopropylmethoxy)-5-[(dimethylamino)methyl]phenyl}amino)carbonyl]-2-(1H-indol-3-yl)propyl]-4-(4-methoxyphenyl)piperazine-1-carboxamide

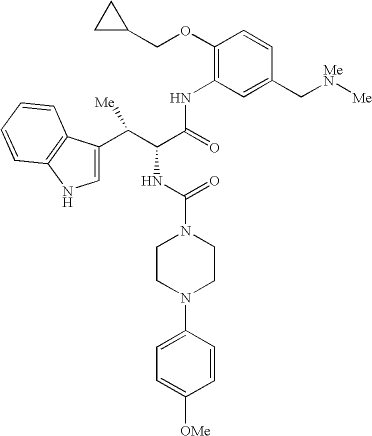

LC/MS (ESI) m/z 639 (M+H⁺)

### Example 264

### N-[(1R,2S)-1-[({2-(cyclopropylmethoxy)-5-[(dimethylamino)methyl]phenyl}amino)carbonyl]-2-(1H-indol-3-yl)propyl]-4-(2-fluorophenyl)piperazine-1-carboxamide

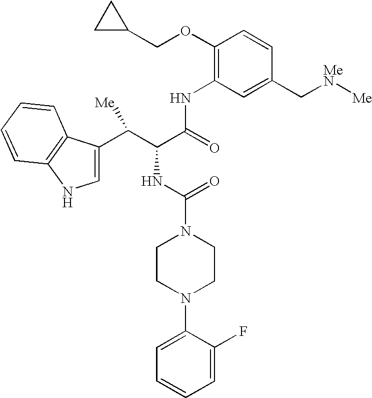

LC/MS (ESI) m/z 627 (M+H⁺).

### Example 265

### 4-cyclohexyl-N-[(1R,2S)-1-[({2-(cyclopropylmethoxy)-5-[(dimethylamino)methyl]phenyl}amino)carbonyl]-2-(1H-indol-3-yl)propyl]piperazine-1-carboxamide

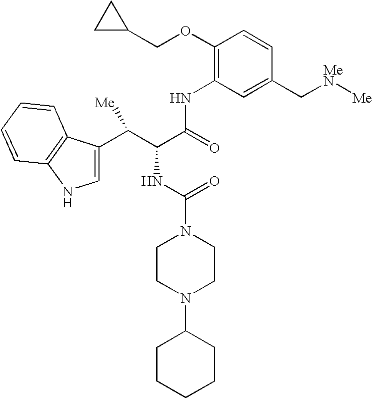

LC/MS (ESI) m/z 615 (M+H⁺)

### Example 266

### N-[(1R,2S)-1-[({2-acetyl-5-[(dimethylamino)methyl]phenyl}amino)carbonyl]-2-(1H-indol-3-yl)propyl]-4-phenylpiperidine-1-carboxamide

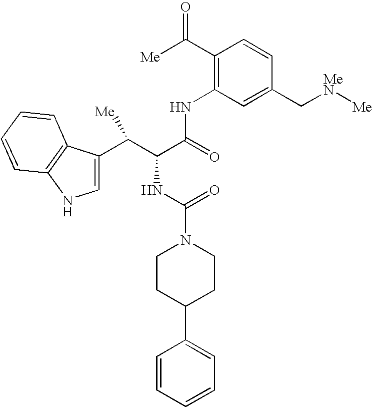

LC/MS (ESI) m/z 580 (M+H⁺).

### Example 267

### N-[(1R,2S)-1-[({2-acetyl-5-[(dimethylamino)methyl]phenyl}amino)carbonyl]-2-(1H-indol-3-yl)propyl]-4-(4-fluorophenyl)piperazine-1-carboxamide

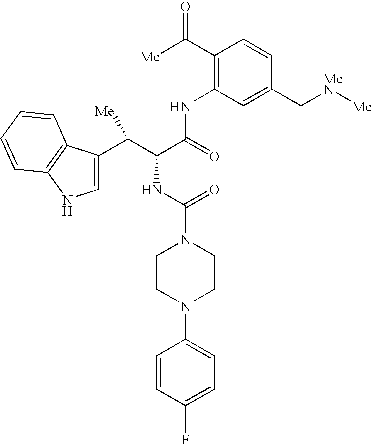

### Example 268

### N-[(1R,2S)-1-[({2-acetyl-5-[(dimethylamino)methyl]phenyl}amino)carbonyl]-2-(1H-indol-3-yl)propyl]-4-(2-methylphenyl)piperazine-1-carboxamide

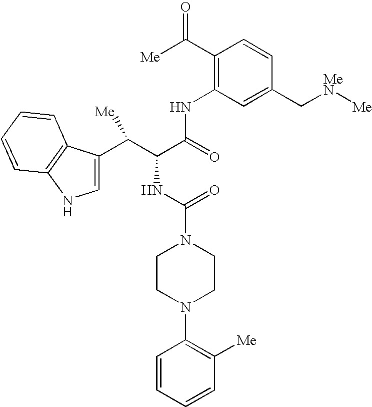

LC/MS (ESI) m/z 595 (M+H⁺)

### Example 269

### N-[(1R,2S)-1-[({2-acetyl-5-[(dimethylamino)methyl]phenyl}amino)carbonyl]-2-(1H-indol-3-yl)propyl]-4-(4-fluoro-2-methylphenyl)piperazine-1-carboxamide

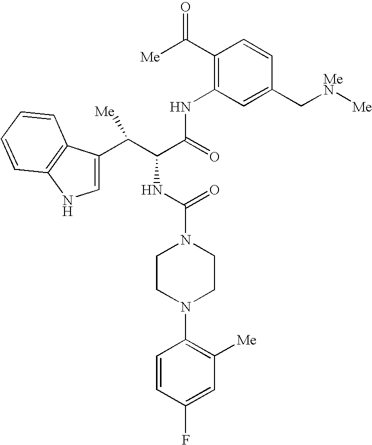

LC/MS (ESI) m/z 613 (M+H⁺).

### Example 270

### N-[(1RS,2RS)-1-[({5-[(dimethylamino)methyl]-2-ethoxyphenyl}amino)carbonyl]-2-(1H-indol-3-yl)propyl]-4-(4-fluorophenyl)piperazine-1-carboxamide

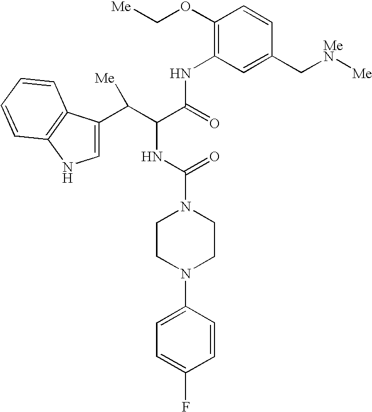

LC/MS (ESI) m/z 601 (M+H⁺)

### Example 271

### N-[(1R,2S)-1-[({5-[(dimethylamino)methyl]-2-ethoxyphenyl}amino)carbonyl]-2-(1H-indol-3-yl)propyl]-2-phenylpyrrolidine-1-carboxamide

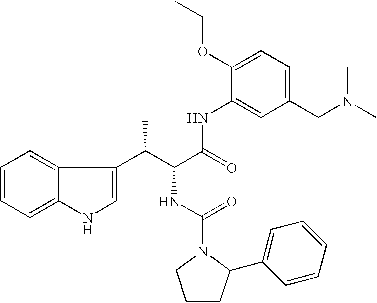

LC/MS (ESI) m/z 568 (M+H⁺).

### Example 272

### N-[(1R,2S)-1-[({5-[(dimethylamino)methyl]-2-ethoxyphenyl}amino)carbonyl]-2-(1H-indol-3-yl)propyl]-2-benzylpyrrolidine-1-carboxamide

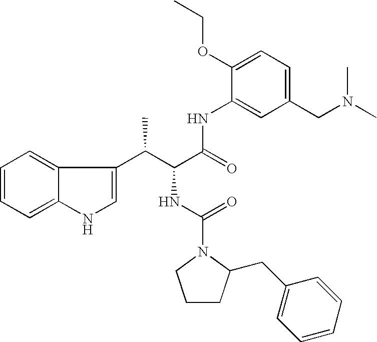

LC/MS (ESI) m/z 582 (M+H⁺).

### Example 273

### N-[(1RS,2SR)-1-({[3-(aminomethyl)phenyl]amino}carbonyl)-2-(1H-indol-3-yl)propyl]-4-(4-fluorophenoxy)-1-piperidinecarboxamide

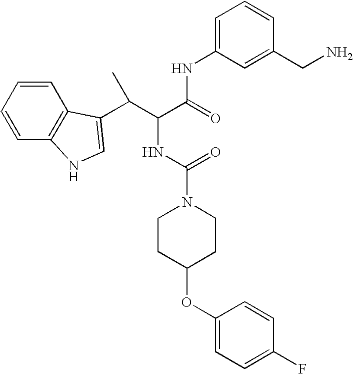

To a mixture of N-(3-aminobenzyl)-2,2,2-trifluoroacetamide (0.18 g, about 0.82 mmol), and (2RS,3SR)-2-(}[4-(4-fluorophenoxy)-1-piperidinyl]carbonyl}amino)-3-(1H-indol-3-yl)butanoic acid (0.35 g, 0.80 mmol), WSC (0.18 g, 0.95 mmol) and HOBt (0.14 g, 0.91 mmol) were added THF (1.0 mL) and acetonitrile (1.0 mL) at room temperature, and the mixture was stirred overnight. The reaction solution was diluted with ethyl acetate and filtered by passing through an alumina layer. The mother liquid was concentrated and the -residue was dissolved in methanol (3.0 mL). 10% Aqueous potassium carbonate solution (1.0 mL) was added at room temperature and the mixture was stirred overnight. The reaction solution was concentrated, water and ethyl acetate were added, and the mixture was subjected to extraction. The organic layer was dried (Na₂SO₄) and concentrated under reduced pressure. The residue was solidified using diethyl ether and a small amount of dichloromethane. The obtained suspension was filtered and the resulting product was washed with a mixture of diethyl ether and dichloromethane and dried under reduced pressure to give the title compound (0.23 g, yield 54%).

LC/MS (ESI) m/z 544 (M+H⁺).

The compound described in the following Example 274 was produced in the similar manner as in Example 273.

### Example 274

### N-[(1RS,2SR)-1-(([4-(aminomethyl)phenyl]amino}carbonyl)-2-(1H-indol-3-yl)propyl]-4-(4-fluorophenoxy)-1-piperidinecarboxamide

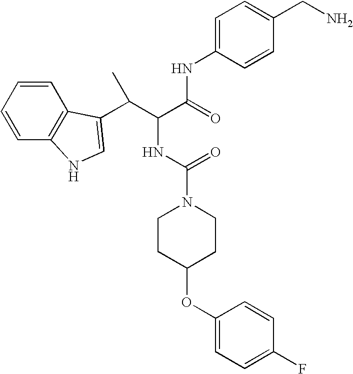

LC/MS (ESI) m/z 544 (M+H⁺).

### Example 275

### 4-(4-fluorophenoxy)-N-((1RS,2SR)-2-(1H-indol-3-yl)-1-{[(2-{[(trifluoroacetyl)amino]methyl}phenyl)amino]carbonyl}propyl)-1-piperidinecarboxamide

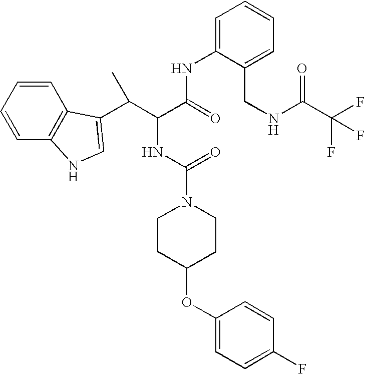

To a mixture of N-(2-aminobenzyl)-2,2,2-trifluoroacetamide (0.17 g, about 0.78 mmol), and (2RS,3SR)-2-(}[4-(4-fluorophenoxy)-1-piperidinyl]carbonyl}amino)-3-(1H-indol-3-yl)butanoic acid (0.34 g, 0.76 mmol), WSC (0.18 g, 0.91 mmol) and HOBt (0.13 g, 0.86 mmol) were added THF (1.0 mL) and acetonitrile (1.0 mL) at room temperature and the mixture was stirred overnight. The reaction solution was diluted with ethyl acetate and filtered by passing through an alumina layer. The mother liquid was concentrated and the residue was purified by alumina column chromatography to give the title compound (0.26 g, yield 54%).

LC/MS (ESI) m/z 640 (M+H⁺).

### Example 276

### N-[(1RS,2SR)-1-({[2-(aminomethyl)phenyl]amino}carbonyl)-2-(1H-indol-3-yl)propyl]-4-(4-fluorophenoxy)-1-piperidinecarboxamide

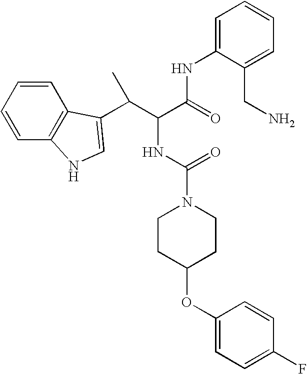

4-(4-Fluorophenoxy)-N-((1RS,2SR)-2-(1H-indol-3-yl)-1-{[(2-{[(trifluoroacetyl)amino]methyl}phenyl)amino]-carbonyl}propyl)-1-piperidinecarboxamide (0.26 g, 0.41 mmol) was dissolved in methanol (3.0 mL). 10% Aqueous potassium carbonate solution (1.0 mL) was added at room temperature and the mixture was stirred overnight. The reaction solution was concentrated, water and ethyl acetate were added and the mixture was subjected to extraction. The organic layer was dried (Na₂SO₄) and concentrated under reduced pressure. The residue was purified by alumina column chromatography to give the title compound (0.061 g, yield 27%).

LC/MS (ESI) m/z 544 (M+H⁺).

### Example 277

### N-[(1RS,2SR)-1-[({2-[(dimethylamino)methyl]phenyl}amino)carbonyl]-2-(1H-indol-3-yl)propyl]-4-(4-fluorophenoxy)-1-piperidinecarboxamide

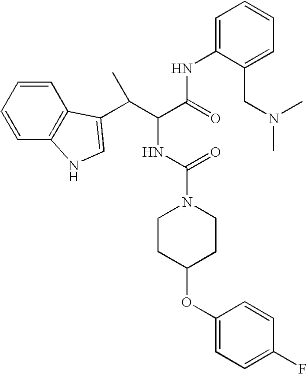

N-[(1RS,2SR)-1-({[2-(Aminomethyl)phenyl]amino}carbonyl)-2-(1H-indol-3-yl)propyl]-4-(4-fluorophenoxy)-1-piperidinecarboxamide (0.043 g, 0.079 mmol) was dissolved in ethanol (1.0 mL) and aqueous solution of 37% formaldehyde (0.025.g, 0.31 mmol) and sodium triacetoxyborohydride (0.042 g, 0.20 mmol) were added. The mixture was stirred overnight at room temperature. To the reaction solution were added saturated aqueous solution of sodium hydrogen carbonate and water and the mixture was stirred for 1 hr. The obtained precipitated products were collected by filtration, washed with water and dried under reduced pressure to give the title compound (0.031 g, yield 80%).

LC/MS (ESI) m/z 572 (M+H⁺).

The compound described in the following Example 278 was produced in the similar manner as in Example 277.

### Example 278

### N-[(1RS,2SR)-1-[({4-[(dimethylamino)methyl]phenyl}amino)carbonyl]-2-(1H-indol-3-yl)propyl]-4-(4-fluorophenoxy)-1-piperidinecarboxamide

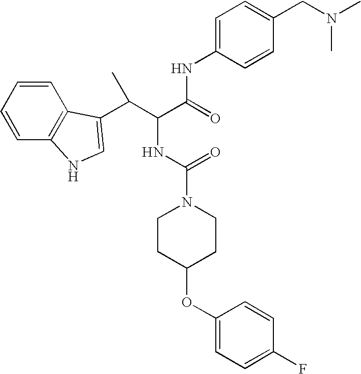

LC/MS (ESI) m/z 572 (M+H⁺).

### Example 279

### tert-butyl {2-[[(2RS,3SR)-2-({[4-(4-fluorophenoxy)-1-piperidinyl]carbonyl}amino)-3-(1H-indol-3-yl)butanoyl](methyl)amino]benzyl}carbamate

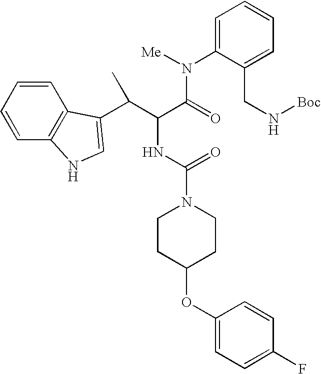

tert-Butyl (2-aminobenzyl)carbamate (0.92 g, 4.2 mmol) and benzotriazole (0.49 g, 4.1 mmol) were dissolved in ethanol (4.0 mL), and aqueous solution of 37% formaldehyde (0.30 mL, 4.0 mmol) was added at room temperature. The mixture was stirred overnight and the solvent was evaporated under reduced pressure. The residue was dissolved in THF (4.0 mL). Sodium borohydride (0.17 g, 4.6 mmol) was added at room temperature and the mixture was stirred for 4 hrs. Sodium borohydride (0.065 g, 1.8 mmol) was further added and the mixture was stirred for 3 hrs. Then, saturated aqueous solution of sodium hydrogen carbonate and ethyl acetate were added and the mixture was subjected to extraction. The organic layer was dried (Na₂SO₄) and concentrated under reduced pressure. The residue was purified by silica gel column chromatography to give tert-butyl[2-(methylamino)benzyl]carbamate (0.69 g) at about 60% purity as a mixture with by-products.

To a mixture of the obtained crude product (0.29 g, about 0.74 mmol), and (2RS,3SR)-2-({[4-(4-fluorophenoxy)-1-piperidinyl]carbonyl}amino)-3-(1H-indol-3-yl)butanoic acid (0.35 g, 0.80 mmol), WSC (0.18 g, 0.93 mmol) and HOBt (0.14 g, 0.91 mmol) were added THF (1.0 mL) and acetonitrile (1.0 mL) at room temperature and the mixture was stirred overnight. To the reaction solution were added saturated aqueous solution of sodium hydrogen carbonate and ethyl acetate and the mixture was subjected to extraction. The organic layer was dried (Na₂SO₄) and concentrated under reduced pressure. The residue was purified by silica gel column chromatography to give the title compound (0.30 g, yield 57%).

LC/MS (ESI) m/z 658 (M+H⁺).

### Example 280

### 4-(4-fluorophenoxy)-N-((1RS,2SR)-2-(1H-indol-3-yl)-1-{[methyl(3-{[(trifluoroacetyl)amino]methyl}phenyl)-amino]carbonyl}propyl)-1-piperidinecarboxamide

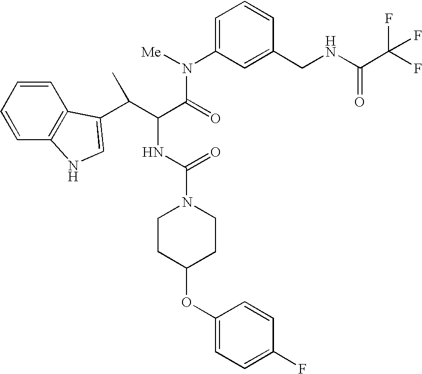

To a mixture of 2,2,2-trifluoro-N-[3-(methylamino)benzyl]acetamide (0.36 g, about 0.82 mmol), and (2RS,3SR)-2-({[4-(4-fluorophenoxy)-1-piperidinyl]carbonyl}amino)-3-(1H-indol-3-yl)butanoic acid (0.36 g, 0.82 mmol), WSC (0.19 g, 0.99 mmol) and HOBt (0.14 g, 0.91 mmol) were added THF (1.0 mL) and acetonitrile (1.0 mL) at room temperature and the mixture was stirred overnight. To the reaction solution were added saturated aqueous solution of sodium hydrogen carbonate and ethyl acetate and the mixture was subjected to extraction. The organic layer was dried (Na₂SO₄) and concentrated under reduced pressure. The residue was purified by silica gel column chromatography to give the title compound (0.50 g, yield 93%).

LC/MS (ESI) m/z 654 (M+H⁺).

The compound described in the following Example 281 was produced in the similar manner as in Example 280.

### Example 281

### 4-(4-fluorophenoxy)-N-((1RS,2SR)-2-(1H-indol-3-yl)-1-{[methyl(4-{[(trifluoroacetyl)amino]methyl}phenyl-amino]carbonyl}propyl)-1-piperidinecarboxamide

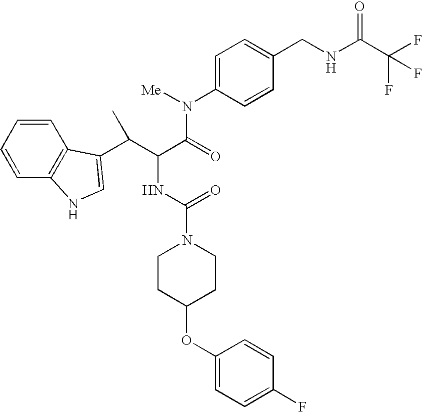

LC/MS (ESI) m/z 654 (M+H⁺).

### Example 282

### N-[(1RS,2SR)-1-{[[2-(aminomethyl)phenyl](methyl)amino]-carbonyl}-2-(1H-indol-3-yl)propyl]-4-(4-fluorophenoxy)-1-piperidinecarboxamide hydrochloride

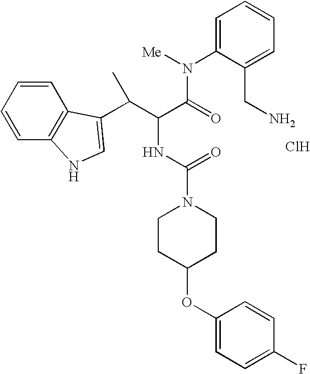

To tert-butyl {2-[[(2RS,3SR)-2-({[4-(4-fluorophenoxy)-1-piperidinyl]carbonyl}amino)-3-(1H-indol-3-yl)butanoyl](methyl)amino]benzyl}carbamate (0.30 g, 0.46 mmol) was added 4N hydrochloric acid-dioxane solution at room temperature and the mixture was stirred for 1 hr. The reaction mixture was concentrated under reduced pressure. To the residue were added 2-propanol and diethyl ether and the obtained suspension was left standing overnight at room temperature. The obtained precipitates were collected by filtration, washed with a mixture of 2-propanol and diethyl ether and dried under reduced pressure to give the title compound as a hygroscopic solid (0.21 g, yield 75%).

LC/MS (ESI) m/z 558 (M+H⁺)—HCl.

### Example 283

### N-[(1RS,2SR)-1-{[[3-(aminomethyl)phenyl](methyl)amino]-carbonyl}-2-(1H-indol-3-yl)propyl]-4-(4-fluorophenoxy)-1-piperidinecarboxamide

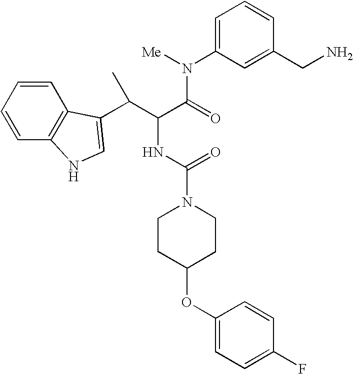

4-(4-Fluorophenoxy)-N-((1RS,2SR)-2-(1H-indol-3-yl)-1-{[methyl(3-{[(trifluoroacetyl)amino]methyl}phenyl)-amino]carbonyl}propyl)-1-piperidinecarboxamide (0.49 g, 0.75 mmol) was dissolved in methanol (5.0 mL) and 10% aqueous potassium carbonate solution (2.0 mL) was added at room temperature. The mixture was stirred for 2 days and methanol was evaporated under reduced pressure from the reaction solution to allow precipitation of products. Water was further added and the obtained suspension was filtered. The resulting product was washed with water and dried under reduced pressure to give the title compound (0.34 g, yield 82%).

LC/MS (ESI) m/z 558 (M+H⁺).

The compound described in the following Example 284 was produced in the similar manner as in Example 283.

### Example 284

### N-[(1RS,2SR)-1-{[[4-(aminomethyl)phenyl](methyl)amino]carbonyl}-2-(1H-indol-3-yl)propyl]-4-(4-fluorophenoxy)-1-piperidinecarboxamide

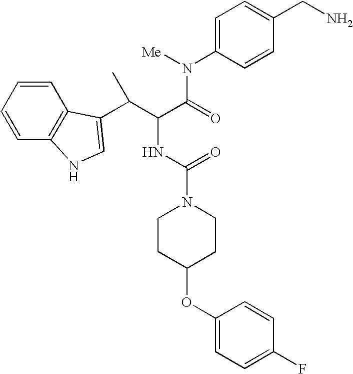

LC/MS (ESI) m/z 558 (M+H⁺).

### Example 285

### N-[(1RS,2SR)-1-{[{2-[(dimethylamino)methyl]phenyl}(methyl)amino]carbonyl}-2-(1H-indol-3-yl)propyl]-4-(4-fluorophenoxy)-1-piperidinecarboxamide

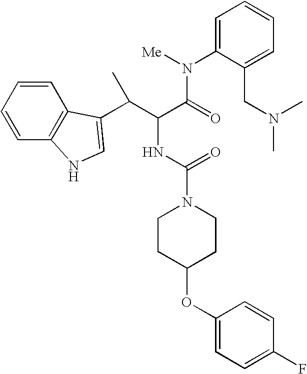

N-[(1RS,2SR) -1-{[[2-(Aminomethyl)phenyl](methyl)amino]carbonyl}-2-(1H-indol-3-yl)propyl]-4-(4-fluorophenoxy)-1-piperidinecarboxamide hydrochloride (0.099 g, 0.17 mmol) was dissolved in ethanol (1.0 mL) and 30% aqueous solution of formaldehyde (0.035 mL, 0.47 mmol) was added at room temperature. After stirring for a while, sodium triacetoxyborohydride (0.12 g, 0.56 mmol) was added and the mixture was stirred overnight. To the reaction solution were added saturated aqueous solution of sodium hydrogen carbonate and water and the mixture was stirred for 2 hrs. The obtained precipitates were collected by filtration, washed with water and dried under reduced pressure to give the title compound (0.086 g, yield 86%).

LC/MS (ESI) m/z 586 (M+H⁺).

The compounds described in the following Examples 286-287 were produced in the similar manner as in Example 285.

### Example 286

### N-[(1RS,2SR)-1-{[{3-[(dimethylamino)methyl]phenyl}(methyl)amino]carbonyl}-2-(1H-indol-3-yl)propyl]-4-(4-fluorophenoxy)-1-piperidinecarboxamide

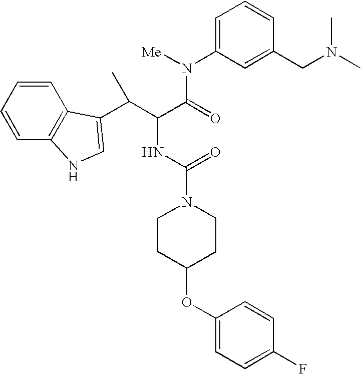

LC/MS (ESI) m/z 586 (M+H⁺).

### Example 287

### N-[(1RS,2SR)-1-{[{4-[(dimethylamino)methyl]phenyl}(methyl)amino]carbonyl}-2-(1H-indol-3-yl)propyl]-4-(4-fluorophenoxy)-1-piperidinecarboxamide

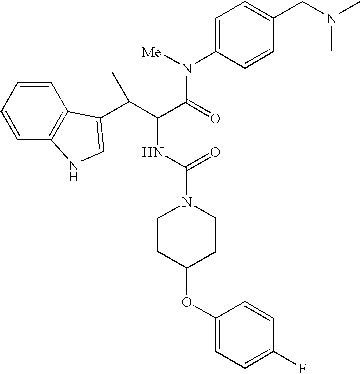

LC/MS (ESI) m/z 586 (M+H⁺).

### Example 288

### tert-butyl {(3-{[(2R,3S)-2-amino-3-(1H-indol-3-yl)butanoyl]amino}benzyl)carbamate

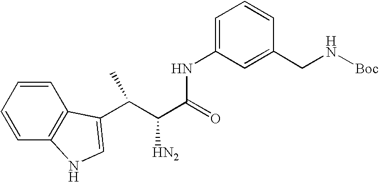

To a mixture of tert-butyl (3-aminobenzyl)carbamate (0.65 g, 2.9 mmol), (2R,3S)-2-{[(9H-fluoren-9-ylmethoxy)carbonyl]amino}-3-(1H-indol-3-yl)butanoic acid (1.4 g, 3.2 mmol), WSC (0.65 g, 3.4 mmol) and HOBt (0.52 g, 3.4 mmol) were added THF (3.0 mL) and acetonitrile (3.0 mL) at room-temperature, and the mixture was stirred overnight. To the reaction solution were added saturated aqueous solution of sodium hydrogen carbonate and ethyl acetate and the mixture was subjected to extraction. The organic layer was dried (Na₂SO₄) and concentrated under reduced pressure. The residue was dissolved in methanol (40 mL), piperidine (4.0 mL) was added at room temperature and the mixture was stirred overnight. The reaction solution was concentrated and purified by silica gel column chromatography. Furthermore, a mixed solvent of diisopropyl ether, dichloromethane, diethyl ether and hexane was used to allow solidification. The solid product was filtered and washed with a mixed solvent of dichloromethane and hexane and dried under reduced pressure to give the title compound (0.82 g, yield 66%).

¹H NMR (300 MHz, CDCl₃) δ ppm: 1.31 (d, J=7.1 Hz, 3 H), 1.47 (s, 9 H), 3.95 (d, J=3.4 Hz, 1 H), 4.07 (d, 1 H), 4.31 (d, J=6.1 Hz, 2 H), 4.88 (s, 1 H), 7.04 (d, J=8.1 Hz, 1 H), 7.07 (d, J=1.5 Hz, 1 H), 7.11-7.18 (m, 1 H), 7.19 -7.24 (m, 1 H), 7.26-7.34 (m, 1 H), 7.39 (d, J=8.1 Hz, 1 H), 7.50-7.56 (m, 2 H), 7.78 (d, J=7.6 Hz, 1 H), 8.17 (s, 1 H), 9.51 (s, 1 H).

### Example 289

### N-[(1R,2S)-1-({[3-(aminomethyl)phenyl]amino}carbonyl)-2-(1H-indol-3-yl)propyl]-1-benzoyl-4-piperidinecarboxamide

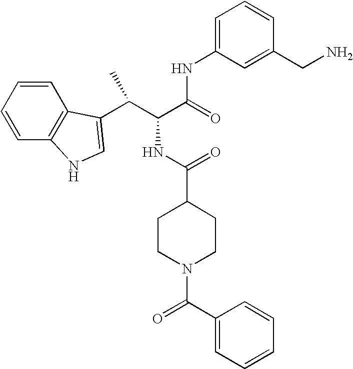

To a mixture of tert-butyl (3-{[(2R,3S)-2-amino-3-(1H-indol-3-yl)butanoyl]amino}benzyl) carbamate (0.20 g, 0.47 mmol), and 1-benzoylpiperidine-4-carboxylic acid (0.12 g, 0.53 mmol), WSC (0.11 g, 0.58 mmol) and HOBt (0.088 g, 0.57 mmol) were added THF (1.0 mL) and acetonitrile (1.0 mL) at room temperature, and the mixture was stirred overnight. To the reaction solution were added saturated aqueous solution of sodium hydrogen carbonate and ethyl acetate and the mixture was subjected to extraction. The organic layer was filtered by passing through a silica gel layer, and concentrated under reduced pressure. The residue was dissolved in dioxane (1.0 mL), and 4N hydrochloric acid-dioxane solution (1.0 mL) was added at room temperature. The mixture was stirred for 30 min. To the reaction solution were added saturated aqueous solution of sodium hydrogen carbonate and a mixed solvent of ethyl acetate-dichloromethane and the mixture was subjected to extraction. The organic layer was dried (MgSO₄) and concentrated under reduced pressure. The residue was purified by HPLC (acetonitrile/water=10/90-100/0, containing 0.1% trifluoroacetic acid). A fraction of the object product was concentrated and neutralized with saturated aqueous solution of sodium hydrogen carbonate to give the title compound (0.084 g, yield 33%).

LC/MS (ESI) m/z 538 (M+H⁺).

### Example 290

### (2R,3S)-N-[3-(aminomethyl)phenyl]-2-{[(1-benzoyl-4-piperidinyl)methyl]amino}-3-(1H-indol-3-yl)butanamide

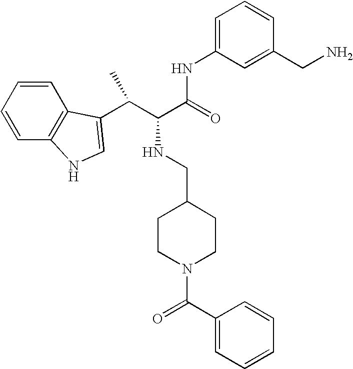

tert-Butyl (3-{[(2R,3S)-2-amino-3-(1H-indol-3-yl)butanoyl]amino}benzyl)carbamate (0.21 g, 0.49 mmol) and 1-benzoylpiperidine-4-carbaldehyde (0.12 mL, 0.57 mmol) were dissolved in ethanol (2.0 mL) and the mixture was stirred at room temperature for 20 min. Sodium triacetoxyborohydride (0.13 g, 0.60 mmol) was added and the mixture was stirred overnight. To the reaction solution were added saturated aqueous solution of sodium hydrogen carbonate and ethyl acetate and the mixture was subjected to extraction. The organic layer was dried (MgSO₄) and concentrated under reduced pressure. The residue was purified by silica gel column chromatography and the obtained crude product was dissolved in dioxane (1.0 mL). 4N Hydrochloric acid-dioxane solution (2.0 mL) was added at room temperature and the mixture was stirred for 2 hrs. The reaction solution was concentrated, aqueous solution of sodium hydrogen carbonate was added to the residue. The obtained suspension was filtered and the resulting product was washed with water and dried under reduced pressure to give the title compound (0.17 g, yield 65%).

LC/MS (ESI) m/z 524 (M+H⁺).

### Example 291

### (2R,3S)-2-{[(1-benzoyl-4-piperidinyl)methyl]amino}-N-{3-[(dimethylamino)methyl]phenyl}-3-(1H-indol-3-yl)butanamide

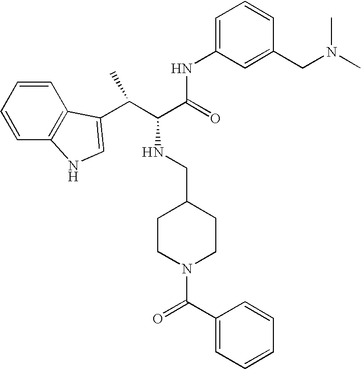

To a mixture of 3-((dimethylamino)methyl)aniline dihydrochloride (0.10 g, 0.45 mmol), and (2R,3S)-2-{[(9H-fluoren-9-ylmethoxy)carbonyl]amino}-3-(1H-indol-3-yl)butanoic acid (0.21 g, 0.47 mmol), WSC (0.10 g, 0.52 mmol) and HOBt (0.088 g, 0.57 mmol) were added THF (1.0 mL) and acetonitrile (1.0 mL) at room temperature. Furthermore, triethylamine (0.15 mL, 1.1 mmol) was added and the mixture was stirred overnight. To the reaction solution were added saturated aqueous solution of sodium hydrogen carbonate and ethyl acetate and the mixture was subjected to extraction. The organic layer was filtered by passing through an alumina layer and concentrated. The residue was dissolved in methanol (2.0 mL), piperidine (0.4 mL) was added at room temperature and the mixture was stirred overnight. The reaction solution was concentrated and purified by silica gel column chromatography. The crude product and 1-benzoyl-4-piperidinecarbaldehyde (0.043 g, 0.20 mmol) were dissolved in ethanol (1.5 mL) and the mixture was stirred at room temperature for 15 min. To the reaction solution was added sodium triacetoxyborohydride (0.062 g, 0.29 mmol) and the mixture was stirred overnight. To the reaction solution were added saturated aqueous solution of sodium hydrogen carbonate and water and the mixture was stirred. The obtained precipitates were collected by filtration, washed with water, dried under reduced pressure and purified by HPLC (acetonitrile/water=10/90-100/0, containing 0.1% trifluoroacetic acid). A fraction of the object product was concentrated and neutralized with saturated aqueous solution of sodium hydrogen carbonate to give the title compound (0.023 g, yield 9%).

LC/MS (ESI) m/z 552 (M+H⁺)

The compound described in the following Example 292 was produced in the similar manner as in Example 1.

### Example 292

### 4-benzoyl-N-[(1R,2S)-1-[({3-[(dimethylamino)methyl]phenyl}amino)carbonyl]-2-(1H-indol-3-yl)propyl]-1-piperazinecarboxamide

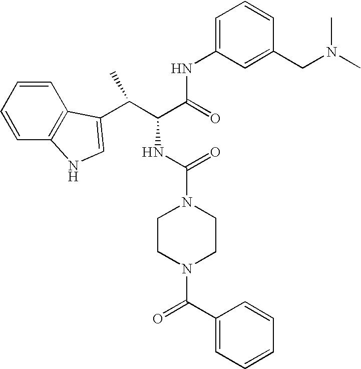

LC/MS (ESI) m/z 567 (M+H⁺)

The compounds described in the following Examples 293-296 were produced in the similar manner as in Example 273.

### Example 293

### N-[(1R,2S)-1-({[3-(aminomethyl)phenyl]amino}carbonyl)-2-(1H-indol-3-yl)propyl]-4-benzoyl-1-piperazinecarboxamide

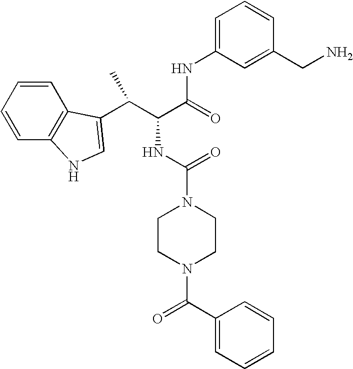

LC/MS (ESI) m/z 539 (M+H⁺).

### Example 294

### N-[(1R,2S)-1-({[3-(2-aminoethyl)phenyl]amino}carbonyl)-2-(1H-indol-3-yl)propyl]-4-(4-fluorophenoxy)-1-piperidinecarboxamide

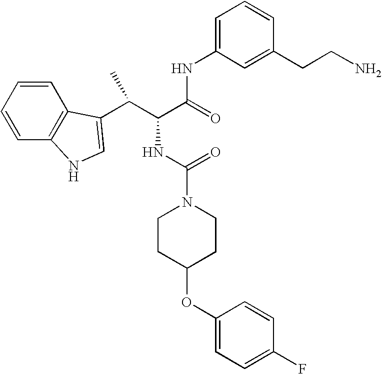

LC/MS (ESI) m/z 558 (M+H⁺).

### Example 295

### N-[(1R,2S)-1-({[3-(2-aminoethyl)phenyl]amino}carbonyl)-2-(1H-indol-3-yl)propyl]-4-benzoyl-1-piperazinecarboxamide

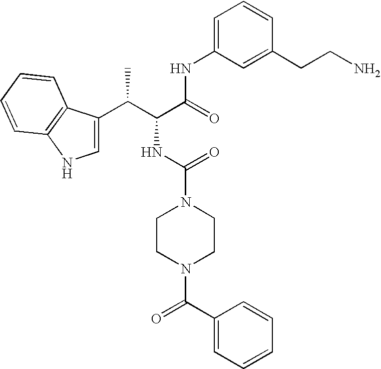

LC/MS (ESI) m/z 553 (M+H⁺).

### Example 296

### N-[(1R,2S)-1-({[3-(2-aminoethyl)phenyl]amino}carbonyl)-2-(1H-indol-3-yl)propyl]-1-benzoyl-4-piperidinecarboxamide

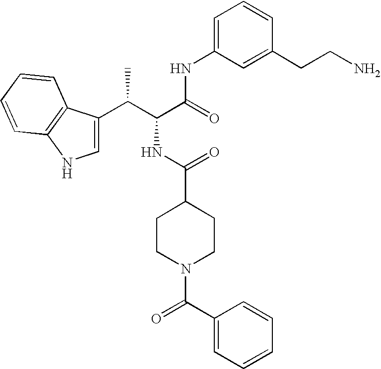

LC/MS (ESI) m/z 5,52 (M+H⁺)

The compounds described in the following Examples 297-299 were produced in the similar manner as in Example 277.

### Example 297

### N-[(1R,2S)-1-[({3-[2-(dimethylamino)ethyl]phenyl}amino)carbonyl]-2-(1H-indol-3-yl)propyl]-4-(4-fluorophenoxy)-1-piperidinecarboxamide

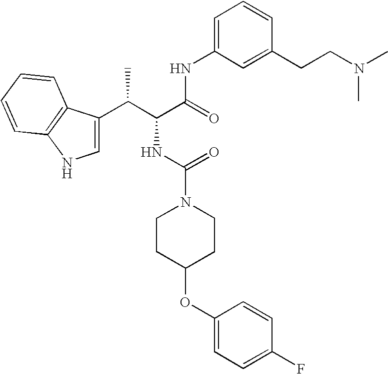

LC/MS (ESI) m/z 586 (M+H⁺)

### Example 298

### 4-benzoyl-N-[(1R,2S)-1-[({3-[2-(dimethylamino)ethyl]phenyl}amino)carbonyl]-2-(1H-indol-3-yl)propyl]-1-piperazinecarboxamide

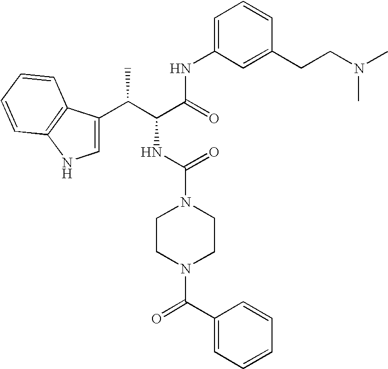

LC/MS (ESI) m/z 581 (M+H⁺).

### Example 299

### 1-benzoyl-N-[(1R,2S)-1-[({3-[2-(dimethylamino)ethyl]phenyl}amino)carbonyl]-2-(1H-indol-3-yl)propyl]-4-piperidinecarboxamide

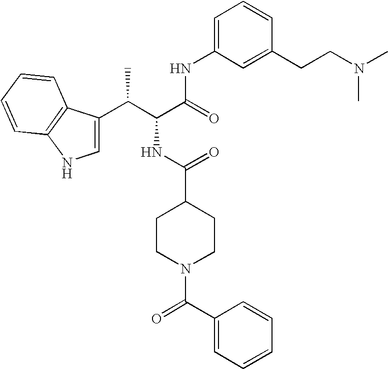

LC/MS (ESI) m/z 580 (M+H⁺).

The compounds described in the following Examples 300-301 were produced in the similar manner as in Example 289.

### Example 300

### N-[(1R,2S)-1-({[3-(aminomethyl)phenyl]amino}carbonyl)-2-(1H-indol-3-yl)propyl]-1-[(1-methyl-1H-indol-2-yl)carbonyl]-4-piperidinecarboxamide

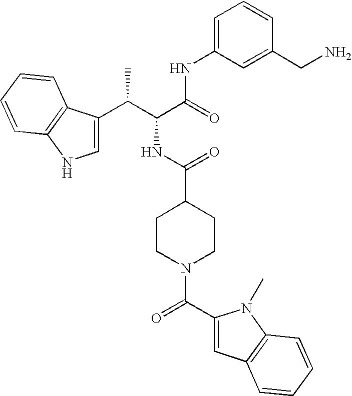

LC/MS (ESI) m/z 591 (M+H⁺).

### Example 301

### (2R,3S)-N-[3-(aminomethyl)phenyl]-2-({[(1-benzoyl-4-piperidinyl)oxy]acetyl}amino)-3-(1H-indol-3-yl)butanamide hydrochloride

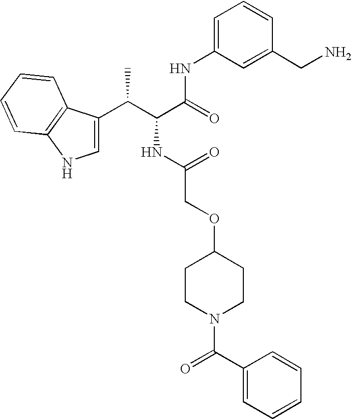

LC/MS (ESI) m/z 568 (M+H⁺)—HCl.

The compounds described in the following Examples 302-303 were produced in the similar manner as in Example 277.

### Example 302

### N-[(1R,2S)-1-[({3-[(dimethylamino)methyl]phenyl}amino)carbonyl]-2-(1H-indol-3-yl)propyl]-1-[(1-methyl-1H-indol-2-yl)carbonyl]-4-piperidinecarboxamide

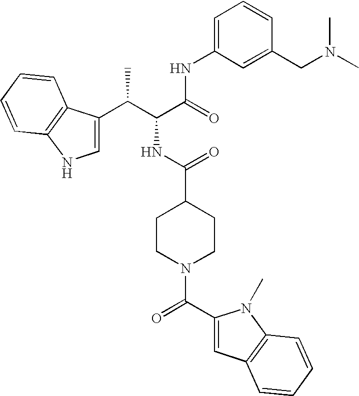

LC/MS (ESI) m/z 619 (M+H⁺).

### Example 303

### (2R,3S)-2-({[(1-benzoyl-4-piperidinyl)oxy]acetyl}amino)-N-{3-[(dimethylamino)methyl]phenyl}-3-(1H-indol-3-yl)butanamide

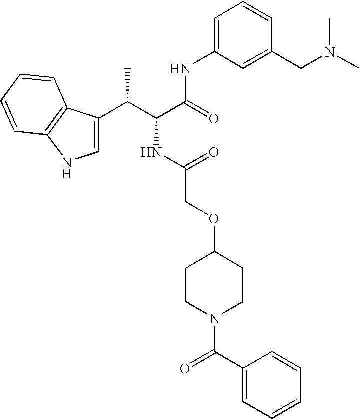

LC/MS (ESI) m/z 596 (M+H⁺).

### Example 304

### N-[(1R,2S)-1-[({3-[(dimethylamino)methyl]phenyl}amino)carbonyl]-2-(1H-indol-3-yl)propyl]-1-piperazinecarboxamide

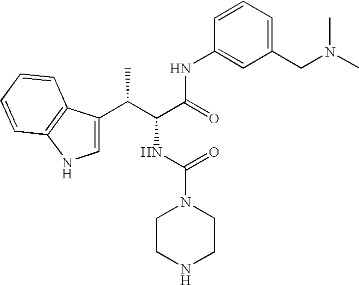

To a suspension of tert-butyl 4-({[(1R,2S)-1-[({3-[(dimethylamino)methyl]phenyl}amino)carbonyl]-2-(1H-indol-3-yl)propyl]amino}carbonyl)-1-piperazinecarboxylate (2.3 g, 4.1 mmol) in dioxane (10 mL) was added 4N hydrochloric acid-dioxane solution (10 mL) at room temperature and the mixture was stirred for 1 hr. To the reaction solution were added saturated aqueous solution of sodium hydrogen carbonate and ethyl acetate and the mixture was subjected to extraction. The organic layer was dried (MgSO₄) and concentrated. To the residue was added trifluoroacetic acid (10 mL) at room temperature and the mixture was stirred for 3 hrs. The reaction solution was concentrated, saturated aqueous solution of sodium hydrogen carbonate and ethyl acetate were added and the mixture was subjected to extraction. The organic layer was dried (MgSO₄) and concentrated. The residue was solidified using ethanol and diethyl ether. The obtained solid product was filtered and washed with diethyl ether and dried under reduced pressure to give the title compound (0.85 g, yield 45%).

LC/MS (ESI) m/z 463 (M+H⁺).

### Example 305

### 4-(cyclohexylcarbonyl)-N-[(1R,2S)-1-[({3-[(dimethylamino)methyl]phenyl}amino)carbonyl]-2-(1H-indol-3-yl)propyl]-1-piperazinecarboxamide

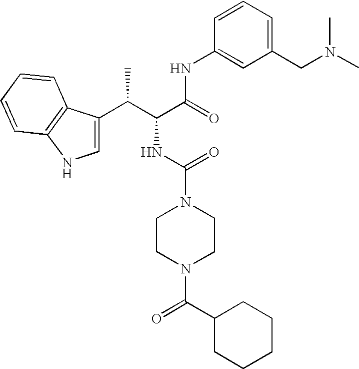

To a mixture of N-[(1R,2S)-1-[({3-[(dimethylamino)methyl]phenyl}amino)carbonyl]-2-(1H-indol-3-yl)propyl]-1-piperazinecarboxamide (0.10 g, 0.22 mmol), cyclohexanecarboxylic acid (0.038 g, 0.30 mmol), WSC (0.056 g, 0.29 mmol) and HOBt (0.045 g, 0.29 mmol) were added THF (0.5 mL) and acetonitrile (0.5 mL) at room temperature and the mixture was stirred overnight. To the reaction solution were added saturated aqueous solution of sodium hydrogen carbonate and ethyl acetate, and the mixture was subjected to extraction. The organic layer was filtered by passing through an aminopropyl silica gel layer and concentrated under reduced pressure. The residue was solidified using dichloromethane and diethyl ether. The obtained solid product was filtered and washed with diethyl ether and dried under reduced pressure to give the title compound (0.069 g, yield 55%).

LC/MS (ESI) m/z 573 (M+H⁺).

The compounds described in the following Examples 306-309 were produced in the similar manner as in Example 305.

### Example 306

### N-[(1R,2S)-1-[({3-[(dimethylamino)methyl]phenyl}amino)carbonyl]-2-(1H-indol-3-yl)propyl]-4-(4-fluorobenzoyl)-1-piperazinecarboxamide

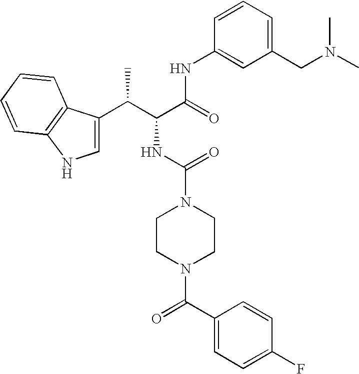

LC/MS (ESI) m/z 585 (M+H⁺).

### Example 307

### N-[(1R,2S)-1-[({3-[(dimethylamino)methyl]phenyl}amino)carbonyl]-2-(1H-indol-3-yl)propyl]-4-(2-naphthoyl)-1-piperazinecarboxamide

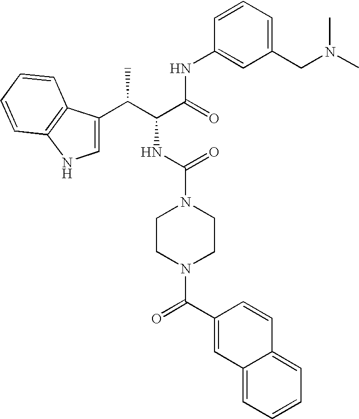

LC/MS (ESI) m/z 617 (M+H⁺)

### Example 308

### N-[(1R,2S)-1-[({3-[(dimethylamino)methyl]phenyl}amino)carbonyl]-2-(1H-indol-3-yl)propyl]-4-(3-pyridinylcarbonyl)-1-piperazinecarboxamide

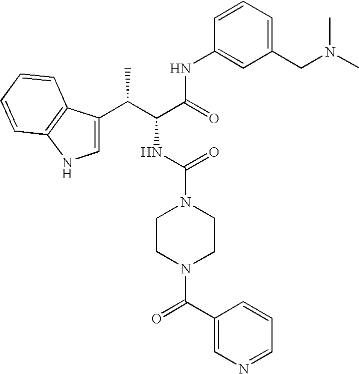

LC/MS (ESI) m/z 568 (M+H⁺).

### Example 309

### N-[(1R,2S)-1-[({3-[(dimethylamino)methyl]phenyl}amino)carbonyl]-2-(1H-indol-3-yl)propyl]-4-(2-furoyl)-1-piperazinecarboxamide

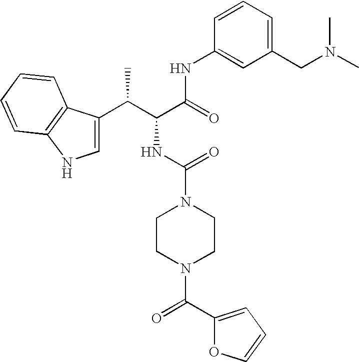

LC/MS (ESI) m/z 557 (M+H⁺).

The compound described in the following Example 310 was produced in the similar manner as in Example 1.

### Example 310

### tert-butyl 4-{3-[((2R,3S)-3-(1H-indol-3-yl)-2-{[(4-phenyl-1-piperidinyl)carbonyl]amino}butanoyl)amino]benzyl}-1-piperazinecarboxylate

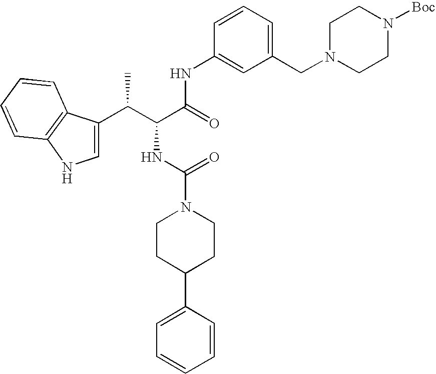

LC/MS (ESI) m/z 679 (M+H⁺)

### Example 311

### N-[(1R,2S)-2-(1H-indol-3-yl)-1-({[3-(1-piperazinylmethyl)phenyl]amino}carbonyl)propyl]-4-phenyl-1-piperidinecarboxamide

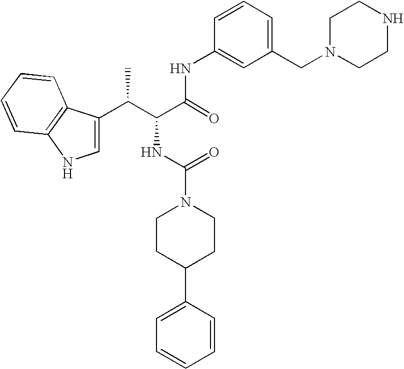

tert-Butyl 4-{3-[((2R,3S)-3-(1H-indol-3-yl)-2-{[(4-phenyl-1-piperidinyl)carbonyl]amino}butanoyl)amino]benzyl}-1-piperazinecarboxylate (0.34 g, 0.50 mmol) was dissolved in dioxane (1.0 mL) and 4N hydrochloric acid-dioxane solution (1.0 mL) was added at room temperature. After stirring for 30 min., 4N hydrochloric acid-dioxane solution (1.0 mL) was added and the mixture was further stirred at room temperature for 1 hr. To the reaction solution were added diethyl ether and water and the mixture was subjected to extraction. To the separated aqueous layer were added saturated aqueous solution of sodium hydrogen carbonate and ethyl acetate and the mixture was subjected to extraction. The organic layer was dried (MgSO₄) and concentrated under reduced pressure. The residue was purified by aminopropyl silica gel column chromatography to give the title compound (0.12 g, 43%).

LC/MS (ESI) m/z 579 (M+H⁺).

The compound described in the following Example 312 was produced in the similar manner as in Example 273.

### Example 312

### N-[(1R,2S)-1-(([3-(2-aminoethyl)phenyl]amino}carbonyl)-2-(1H-indol-3-yl)propyl]-4-phenyl-1-piperidinecarboxamide

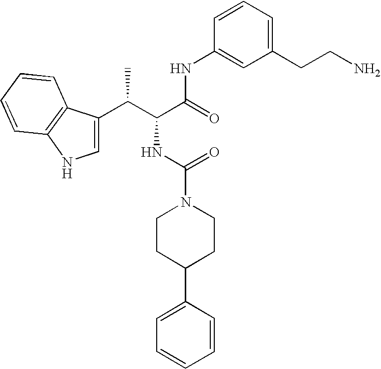

LC/MS (ESI) m/z 524 (M+H⁺).

The compounds described in the following Examples 313-314 were produced in the similar manner as in Example-277.

### Example 313

### N-{(1R,2S)-2-(1H-indol-3-yl)-1-[({3-[(4-methyl-1-piperazinyl)methyl]phenyl}amino)carbonyl]propyl}-4-phenyl-1-piperidinecarboxamide

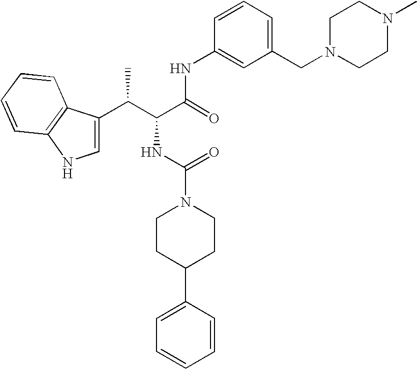

LC/MS (ESI) m/z 593 (M+H⁺).

### Example 314

### N-[(1R,2S)-1-[({3-[2-(dimethylamino)ethyl]phenyl}amino)carbonyl]-2-(1H-indol-3-yl)propyl]-4-phenyl-1-piperidinecarboxamide

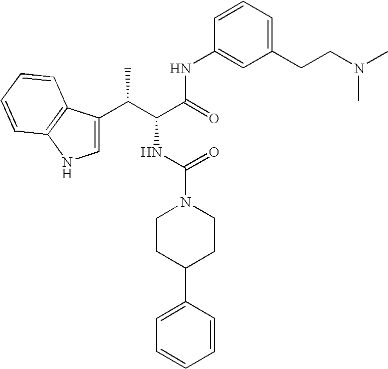

LC/MS (ESI) m/z 552 (M+H⁺).

The compound described in the following Example 315 was produced in the similar manner as in Example 273.

### Example 315

### N-[(1R,2S)-1-({[4-(2-aminoethyl)phenyl]amino}carbonyl)-2-(1H-indol-3-yl)propyl]-4-phenyl-1-piperidinecarboxamide

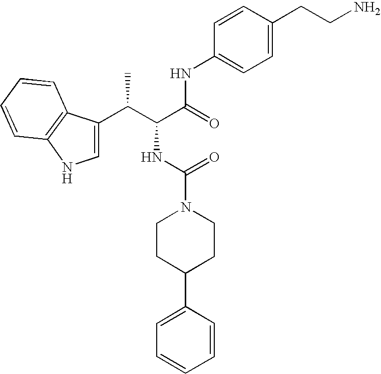

LC/MS (ESI) m/z 524 (M+H⁺).

### Example 316

### 4-cyclohexyl-N-[(1R,2S)-1-[({3-[(dimethylamino)methyl]phenyl}amino)carbonyl]-2-(1H-indol-3-yl)propyl]-1-piperazinecarboxamide

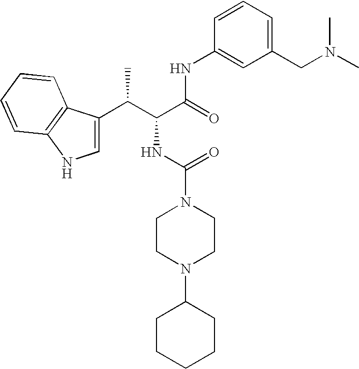

N-[(1R,2S)-1-[({3-[(Dimethylamino)methyl]phenyl}amino)carbonyl]-2-(1H-indol-3-yl)propyl]-1-piperazinecarboxamide (0.10 g, 0.22 mmol) and cyclohexanone (0.034 mL, 0.32 mmol) were dissolved in ethanol (1.0 mL) and the mixture was stirred at room temperature for 15 min. Sodium triacetoxyborohydride (0.11 g, 0.50 mmol) was added and the mixture was stirred overnight. Sodium triacetoxyborohydride (0.088 g, 0.41 mmol) and acetic acid (0.03 mL) were further added and the mixture was stirred at 60° C. for 3 hrs and 70° C. overnight. To the reaction solution were added saturated aqueous solution of sodium hydrogen carbonate and ethyl acetate and the mixture was subjected to extraction. The organic layer was filtered by passing through an aminopropyl silica gel layer and the mother liquid was concentrated. Ethyl acetate and hexane were added to the residue to solidify the product and the obtained solid product was filtered, washed with hexane and dried under reduced pressure to give the title compound (0.030 g, yield 25%).

LC/MS (ESI) m/z 545 (M+H⁺).

The compound described in the following Example 317 was produced in the similar manner as in Example 277.

### Example 317

### N-[(1R,2S)-1-[({4-[2-(dimethylamino)ethyl]phenyl}amino)carbonyl]-2-(1H-indol-3-yl)propyl]-4-phenyl-1-piperidinecarboxamide

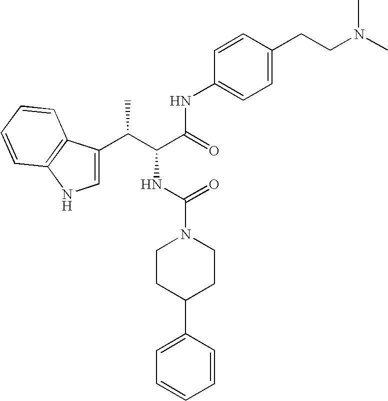

LC/MS (ESI) m/z 552 (M+H⁺).

The compound described in the following Example 318 was produced in the similar manner as in Example 273.

### Example 318

### N-[(1R,2S)-1-({[2-(2-aminoethyl)phenyl]amino}carbonyl)-2-(1H-indol-3-yl)propyl]-4-phenyl-1-piperidinecarboxamide

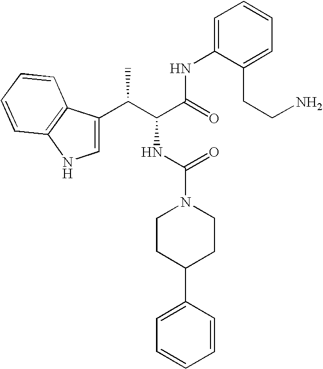

LC/MS (ESI) m/z 524 (M+H⁺).

The compound described in the following Example 319 was produced in the similar manner as in Example 277.

### Example 319

### N-[(1R,2S)-1-[({2-[2-(dimethylamino)ethyl]phenyl}amino)carbonyl]-2-(1H-indol-3-yl)propyl]-4-phenyl-1-piperidinecarboxamide

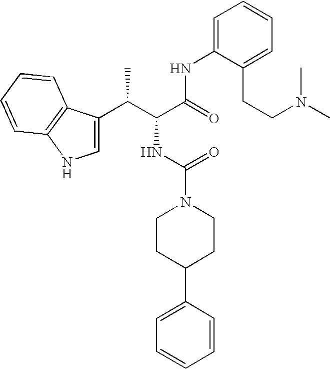

LC/MS (ESI) m/z 552 (M+H⁺).

The compound described in the following Example 320 was produced in the similar manner as in Example 273.

### Example 320

### N-[(1R,2S)-1-({[3-(3-aminopropyl)phenyl]amino}carbonyl)-2-(1H-indol-3-yl)propyl]-4-phenyl-1-piperidinecarboxamide

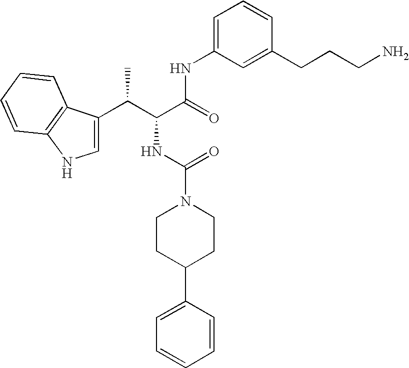

LC/MS (ESI) m/z 538 (M+H⁺)

The compound described in the following Example 321 was produced in the similar manner as in Example 277.

### Example 321

### N-[(1R,2S)-1-[({3-[3-(dimethylamino)propyl]phenyl}amino)carbonyl]-2-(1H-indol-3-yl)propyl]-4-phenyl-1-piperidinecarboxamide

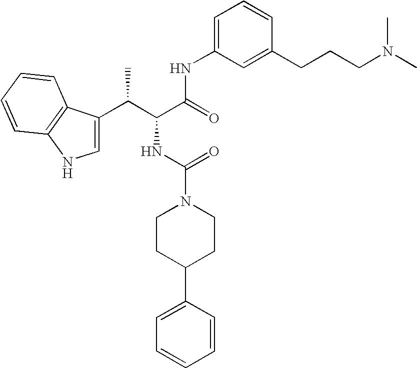

LC/MS (ESI) m/z 566 (M+H⁺).

The compound described in the following Example 322 was produced in the similar manner as in Example 273.

### Example 322

### N-[(1R,2S)-1-({[5-(2-aminoethyl)-2-methoxyphenyl]amino}carbonyl)-2-(1H-indol-3-yl)propyl]-4-(4-fluorophenoxy)-1-piperidinecarboxamide

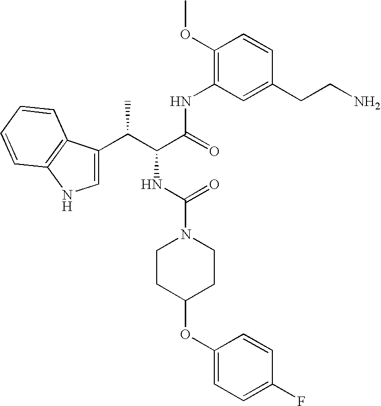

LC/MS (ESI) m/z 588 (M+H⁺).

The compounds described in the following Examples 323-325 were produced in the similar manner as in Example 1.

### Example 323

### N-[(1R,2S)-1-[({5-[(dimethylamino)methyl]-2-methoxyphenyl}amino)carbonyl]-2-(1H-indol-3-yl)propyl]-4-(4-fluorophenyl)-1-piperidinecarboxamide

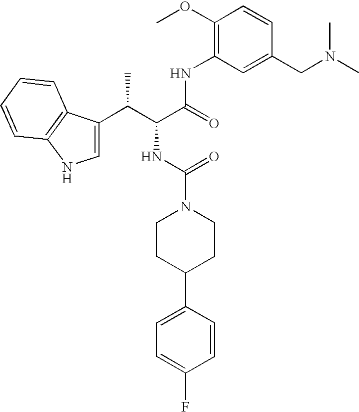

LC/MS (ESI) m/z 586 (M+H⁺).

### Example 324

### N-[(1R,2S)-1-[({5-[(dimethylamino)methyl]-2-ethoxyphenyl}amino)carbonyl]-2-(1H-indol-3-yl)propyl]-4-(4-fluorophenyl)-1-piperidinecarboxamide

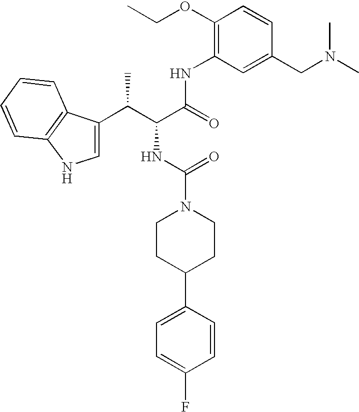

LC/MS (ESI) m/z 600 (M+H⁺).

### Example 325

### N-[(1R,2S)-1-[({5-[(dimethylamino)methyl]-2-ethoxyphenyl}amino)carbonyl]-2-(1H-indol-3-yl)propyl]-4-hydroxy-4-phenyl-1-piperidinecarboxamide

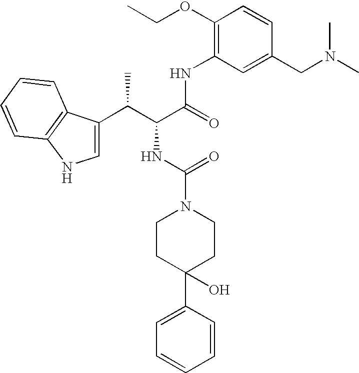

LC/MS (ESI) m/z 598 (M+H⁺).

The compounds described in the following Examples 326-328 were produced in the similar manner as in Example 273.

### Example 326

### N-[(1R,2S)-1-[({2-ethoxy-5-[(methylamino)methyl]phenyl}amino)carbonyl]-2-(1H-indol-3-yl)propyl]-4-phenyl-1-piperidinecarboxamide

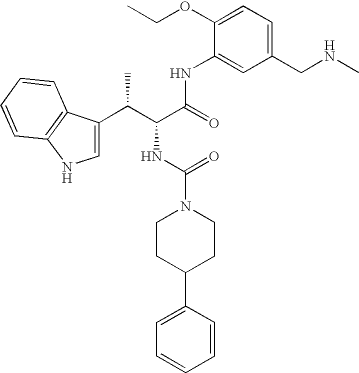

LC/MS (ESI) m/z 568 (M+H⁺).

### Example 327

### N-[(1R,2S)-1-[({2-ethoxy-5-[(methylamino)methyl]phenyl}amino)carbonyl]-2-(1H-indol-3-yl)propyl]-4-(4-fluorophenyl)-1-piper azinecarboxamide

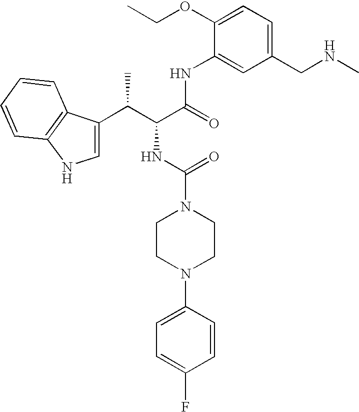

LC/MS (ESI) m/z 587 (M+H⁺).

### Example 328

### N-[(1R,2S)-1-[({2-ethoxy-5-[(methylamino)methyl]phenyl}amino)carbonyl]-2-(1H-indol-3-yl)propyl]-4-(4-fluoro-2-methylphenyl)-1-piperazinecarboxamide

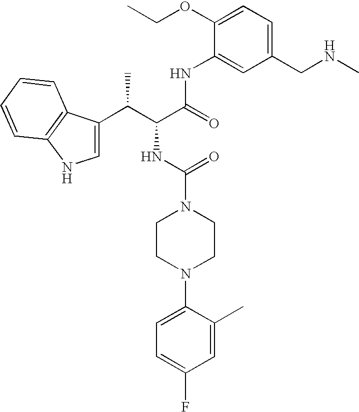

LC/MS (ESI) m/z 601 (M+H⁺).

The compound described in the following Example 329 was produced in the similar manner as in Example 1.

### Example 329

### tert-butyl 4-({[(1R,2S)-1-[({5-[(dimethylamino)methyl]-2-ethoxyphenyl}amino)carbonyl]-2-(1H-indol-3-yl)propyl]amino}carbonyl)-1-piperazinecarboxylate

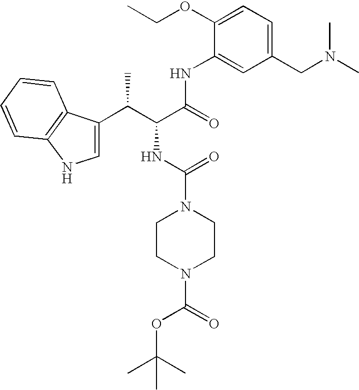

LC/MS (ESI) m/z 607 (M+H⁺).

### Example 330

### 4-(cyclopropylcarbonyl)-N-[(1R,2S)-1-[({5-[(dimethylamino)methyl]-2-ethoxyphenyl}amino)carbonyl]-2- (1H-indol-3-yl)propyl]-1-piperazinecarboxamide

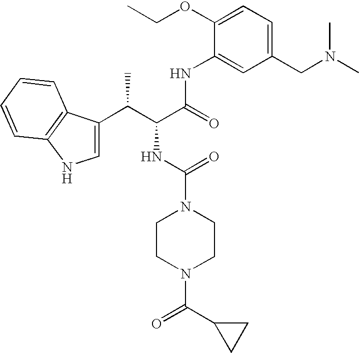

tert-Butyl 4-({[(1R,2S)-1-[({5-[(dimethylamino)methyl]-2-ethoxyphenyl}amino)carbonyl]-2-(1H-indol-3-yl)propyl]amino}carbonyl)-1-piperazinecarboxylate (0.18 g, 0.3 mmol) was dissolved in ethyl acetate (1.0 mL) and 4N hydrochloric acid-ethyl acetate solution (2.0 mL) was added at room temperature and the mixture was stirred for 20 min. The obtained suspension was concentrated, dried under reduced pressure and dissolved in THF (1.0 mL), acetonitrile (1.0 mL) and triethylamine (1.0 mL). To the obtained solution were added cyclopropanecarboxylic acid (0.060 mL, 0.75 mmol), WSC (0.12 g, 0.64 mmol) and HOBt (0.092 g, 0.60 mmol) and the mixture was stirred overnight at room temperature. To the reaction solution were added saturated aqueous solution of sodium hydrogen carbonate and ethyl acetate and the mixture was subjected to extraction. The organic layer was dried (Na₂SO₄) and concentrated. The residue was purified by HPLC (acetonitrile/water=10/90-100/0, containing 0.1% trifluoroacetic acid). A fraction of the object substance was concentrated and neutralized with saturated aqueous solution of sodium hydrogen carbonate to give the title compound (0.040 g, yield 23%) and 4-acetyl-N-[(1R,2S)-1-[({5-[(dimethylamino)methyl]-2-ethoxyphenyl}amino)carbonyl]-2-(1H-indol-3-yl)propyl]-1-piperazinecarboxamide (Example 331) as a by-product (0.044 g, yield 27%).

LC/MS (ESI) m/z 575 (M+H⁺).

The compound described in the following Example 331 was produced in the similar manner as in Example 330.

### Example 331

### 4-acetyl-N-[(1R,2S)-1-[({5-[(dimethylamino)methyl]-2-ethoxyphenyl}amino)carbonyl]-2-(1H-indol-3-yl)propyl]-1-piperazinecarboxamide

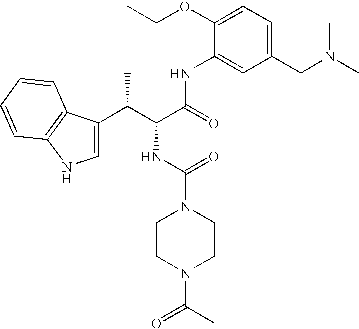

LC/MS (ESI) m/z 549 (M+H⁺).

### Example 332

### 4-cyclobutyl-N-[(1R,2S)-1-[({5-[(dimethylamino)methyl]-2-ethoxyphenyl}amino)carbonyl]-2-(1H-indol-3-yl)propyl]-1-piperazinecarboxamide

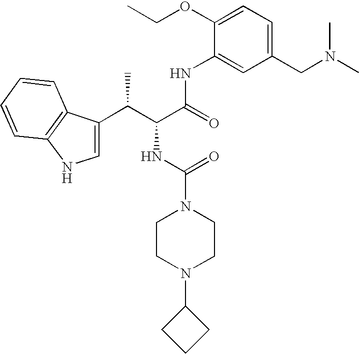

tert-Butyl 4-({[(1R,2S)-1-[({5-[(dimethylamino)methyl]-2-ethoxyphenyl}amino)carbonyl]-2-(1H-indol-3-yl)propyl]amino}carbonyl)-1-piperazinecarboxylate (0.18 g, 0.3 mmol) was dissolved in ethyl acetate (1.0 mL) and 4N hydrochloric acid-dioxane solution (1.0 mL) was added at room temperature. The mixture was stirred for 30 min. The obtained suspension was concentrated, dried under reduced pressure and dissolved in ethanol (1.0 mL). Cyclobutanone (0.030 mL, 0.4 mmol) was added at room temperature and the mixture was stirred for 15 min. To the reaction solution was added sodium triacetoxyborohydride (0.26 g,. 1.2 mmol) and the mixture was stirred overnight. To the reaction solution were added cyclobutanone (0.060 mL, 0.8 mmol) and sodium triacetoxyborohydride (0.21 g, 0.95 mmol) and the mixture was stirred at room temperature for 3 hrs. To the reaction solution were added saturated aqueous solution of sodium hydrogen carbonate and ethyl acetate and the mixture was subjected to extraction. The organic layer was dried (Na₂SO₄) and concentrated. The residue was purified by HPLC (acetonitrile/water=10/90-100/0, containing 0.1% trifluoroacetic acid). A fraction of the object substance was concentrated and neutralized with saturated aqueous solution of sodium hydrogen carbonate to give the title compound (0.072 g, yield 43%).

LC/MS (ESI) m/z 561 (M+H⁺).

The compound described in the following Example 333 was produced in the similar manner as in Example 332.

### Example 333

### 4-cyclopentyl-N-[(1R,2S)-1-[({5-[(dimethylamino)methyl]-2-ethoxyphenyl}amino)carbonyl]-2-(1H-indol-3-yl)propyl]-1-piperazinecarboxamide

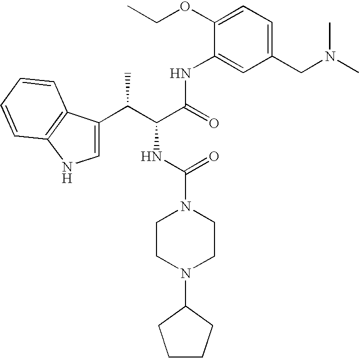

LC/MS (ESI) m/z 575 (M+H⁺).

### Example 334

### (2R,3S)-2-amino-N-{3-[(dimethylamino)methyl]phenyl}-3-(1H-indol-3-yl)butanamide dihydrochloride

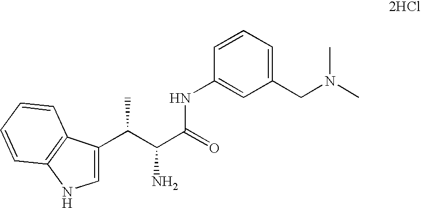

A mixture of (2R,3S)-2-[(tert-butoxycarbonyl)amino]-3-(1H-indol-3-yl)butanoic acid (637 mg, 2.00 mmol), and (3-aminobenzyl)dimethylamine dihydrochloride (442 mg, 1.98 mmol), WSC (575 mg, 3.00 mmol), HOBt (398 mg, 2.60 mmol) and triethylamine (0.588 mL, 4.00 mmol) in THF (5.0 mL)-acetonitrile (5.0 mL) was stirred at room temperature for 16 hrs. To the reaction solution were added saturated solution of sodium carbonate and ethyl acetate and the mixture was subjected to extraction. The organic layer was dried (MgSO₄) and the solvent was evaporated. The residue was purified by silica gel column chromatography (developing solvent: hexane/ethyl acetate 5/1-1/1) to give a pale-yellow amorphous powder.

To a solution (50 mL) of the obtained product in dioxane was added 4N hydrochloric acid-dioxane solution (1.0 mL) at room temperature, and the mixture was stirred for 30 min. The solvent was evaporated and ethyl acetate-diethyl ether was added to the residue to give a suspension. The resulting precipitates were collected by filtration and dried to give the title compound (558 mg, yield 67%) as a pale brown powder.

LC/MS (ESI) m/z 351 (M+H⁺)-2HCl.

### Formulation Example 1

| | | |
|---|---|---|
| (1) Compound obtained in Example 1 | 50.0 | mg |
| (2) Lactose | 34.0 | mg |
| (3) Corn Starch | 10.6 | mg |
| (4) Corn Starch (pasty) | 5.0 | mg |
| (5) Magnesium Stearate | 0.4 | mg |
| (6) Carboxymethyl Cellulose Calcium | 20.0 | mg |
| Total | 120.0 | mg |

The above (1) to (6) were admixed in an ordinary manner, and tabletted using a tabletting machine, to obtain tablets.

### Experimental Example 1

Measurement of the Binding Inhibition Rate of ¹²⁵I-Somatostatin

The receptor binding inhibition rate (%) of the subject compound was calculated using human.somatostatin receptor 2 expressing CHO cells and SSTR2-HS5-9 described in WO02/16350.

First, SSTR2-HS5-9 (1×10⁹) was floated on a phosphate buffered saline supplemented with 5 mM EDTA (PBS-EDTA) and centrifuged. To the cell pellets was added 10 ml of a homogenate buffer for cells (10 mM NaHCO₃, 5 mM EDTA, pH 7.5), which was homogenated using a Polytron homogenizer. The supernatant obtained by centrifugation at 400×g for 15 minutes was further centrifuged at 100,000×g for 1 hour to give a precipitate of the membrane fraction. The precipitates ere suspended in 2 ml of a buffer solution for assay [25 mM Tris-HCl, 1 mM EDTA (Ethylenediaminetetraacetic Acid), 0.1% BSA (Bovine Serum Albumin), 0.25 mM PMSF (Phenylmethylsulfonyl Fluoride), 1 μg/ml pepstatin, 20 μg/ml leupeptin, 10 μg/ml Phosphoramidone, pH 7.5], which was centrifuged at 100,000×g for 1 hour. The membrane fraction recovered as precipitates was suspended again in 20 ml of the buffer solution for assay, which was placed in tubes and stored at −80° C. The suspension was thawed and used at every use.

The thus-obtained membrane fraction of SSTR2-HS5-9 cells was diluted with a buffer solution for assay [25 mM Tris-HCl, 1 mM EDTA (Ethylenediaminetetraacetic Acid), 0.1% BSA (Bovine Serum Albumin), 0.25 mM PMSF (Phenylmethylsulfonyl Fluoride), 1 μg/ml pepstatin, 20 μg/ml leupeptin, 10 μg/ml Phosphoramidone, pH 7.51 to adjust the concentration to 3 μg/ml. The diluate was placed in tubes each in quantity of 173 μl. To this were simultaneously added 2 μl of a solution of a subject compound in DMSO and 25 μl of a 200 pM radioisotope-labeled somatostatin-14 (¹²⁵I-somatostatin-14: Amersham). For measurement of the maximum binding, a reaction mixture added with 2 μl of DMSO and 25 μl of a 200 pM ¹²⁵I-somatostatin was prepared. For measurement of non-specific binding, a reaction mixture added with 2 μl of a 100 μm somatostatin solution in DMSO and 25 μl of a 200 pM ¹²⁵I-somatostatin-14 solution was prepared at the same time. The mixtures were allowed to react at 25° C. for 60 minutes. Then, the reaction mixture was filtered by aspiration using a Whatman glass filter (GF-B) treated with polyethylenimine. After filtration, the radioactivity of ¹²⁵I-somatostatin-14 remaining on the filter paper was measured by a γ-counter. The binding inhibition rate (%) of each subject compound was calculated by the following formula:
(TB−SB)/(TB−NSB)×100
SB: radioactivity when a subject compound was added
TB: maximum binding radioactivity
NSB: non-specific binding radioactivity

The results are shown in Table 1.

TABLE 1

| Example No. | binding inhibition rate (%) |
|---|---|
| 1 | 98 |
| 2 | 100 |
| 54 | 100 |
| 67 | 96 |

This shows that the compound (I) of the present invention has a somatostatin receptor binding inhibition activity.

### Experimental Example 2

Glucagon Secretion Inhibitory Activity Test (Rat)

A 0.5% methylcellulose suspension containing the subject compound (3 mg/kg body weight) (compound administration group) or a 0.5% methylcellulose suspension (compound non-administration group) was orally administered to SD rats (male, 7 weeks of age) after fasting overnight, and 120 minutes later, insulin (2 U/kg body weight, Novo Nordisk) was subcutaneously administered. After 30 minutes from the insulin administration, the blood was drawn from the vein of eyegrounds of the rats using capillary, and blood plasma was separated by centrifugation. The concentration of glucagon in the obtained blood plasma was measured by radioimmunoassay using a Daiichi glucagon kit (Daiichi Isotope). As a non-treated group, the concentration of glucagon in the blood plasma of rat of the compound non-administration group free of insulin administration was measured in the same manner as above.

The difference between the concentration of glucagon of the non-treated group and the concentration of glucagon of the compound non-administration group or the compound administration group was each calculated and the percentage of “the difference between the concentration of glucagon of the compound administration group and the concentration of glucagon of the non-treated group” relative to the “difference between the concentration of glucagon of the compound non-administration group and the concentration of glucagon of the non-treated group” as 100% was determined as “glucagon secretion (% of control)”. The results are shown in Table 2.

TABLE 2

| Example No. | zglucagon secretion (% of control) |
|---|---|
| 1 | 9.5 |

This shows that the compound (I) of the present invention has a glucagon secretion inhibitory activity.

### INDUSTRIAL APPLICABILITY

The compound of the present invention has an excellent somatostatin receptor binding inhibition activity with low toxicity.

Therefore, the compound of the present invention is useful for disorders of an intracellular signal transduction system (e.g., diseases accompanied by excess sthenia or suppression, etc.); diseases accompanied by disorders of regulating cell proliferation; diseases accompanied by disorders of production and/or secretion of hormones, growth factors, or physiologically active substances, etc.; in a mammal.

This application is based on patent application Nos. 002-335661 and 2003-76435 filed in Japan, the contents of which are hereby incorporated by reference.

1. A compound of the formula: 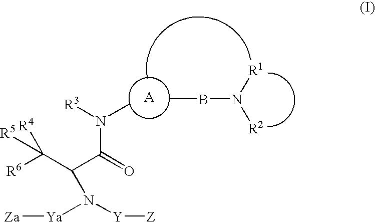
wherein
ring A represents an aromatic ring optionally having substituents;
B, Y and Ya are the same or different and each represents a bond or a spacer having a main chain of 1 to 6 atoms;
R¹ and R² are the same or different and each represents a hydrogen atom, a hydrocarbon group optionally having substituents or a heterocyclic group optionally having substituents, or R¹ and R², together with the adjacent nitrogen atom, form a nitrogen-containing heterocyclic ring optionally having substituents, or R¹ is linked with ring A together with the adjacent nitrogen atom and B to form a 5- to 7-membered nitrogen-containing heterocyclic ring;
R³ represents a hydrogen atom, a hydrocarbon group optionally having substituents or a heterocyclic group optionally having substituents;
R⁴ and R⁵ are the same or different and each represents a hydrogen atom or a hydrocarbon group optionally having substituents, or R⁴ and R⁵, together with the adjacent carbon atom, form a ring optionally having substituents;
R⁶ represents an indolyl group optionally having substituents; and
Z and Za are the same or different and each represents a hydrogen atom, a halogen atom or a cyclic group optionally having substituents; or a salt thereof.

2. A prodrug of the compound according to claim 1 or a salt thereof.

3. The compound according to claim 1, wherein R³ is a hydrogen atom or a C₁₋₆ alkyl optionally having substituents.

4. The compound according to claim 1, wherein one of R⁴ and R⁵ is a hydrogen atom, and the other is a C₁₋₆ alkyl optionally having substituents.

5. The compound according to claim 1, wherein Z is a cyclic group optionally having substituents.

6. The compound according to claim 5, wherein the cyclic group is piperidinyl or piperazinyl.

7. The compound according to claim 5, wherein Z is piperidinyl or piperazinyl, each of which is substituted by a group of the formula: -Yd-Ara wherein Yd represents a bond or a spacer having a main chain of 1 to 6 atoms, and Ara represents a monocyclic group optionally having substituents.

8. The compound according to claim 1, wherein Ya is a bond, and Za is a hydrogen atom.

9. The compound according to claim 1, wherein B is a C₁₋₆ alkylene.

10. The compound according to claim 1, wherein the aromatic ring represented by ring A is benzene.

11. The compound according to claim 1, wherein R¹ and R² are C₁₋₆ alkyl.

12. The compound according to claim 1, wherein Y is —CO—.

13. The compound according to claim 1, which is
N-((1R, 2S)-1-(((5-((dimethylamino)methyl)-2-((methylamino)carbonyl)phenyl)amino)carbonyl)-2-(1H-indol-3-yl)propyl)-4-(2-methylphenyl)-1-piperidinecarboxamide;
N-((1R,2S)-1-(((2-((dimethylamino)carbonyl)-5-((dimethylamino)methyl)phenyl)amino)carbonyl)-2-(1H-indol-3-yl)propyl)-4-(4-fluorophenyl)-1-piperidinecarboxamide;
N-((1R,2S)-1-(((5-((dimethylamino)methyl)-2-methoxyphenyl)amino)carbonyl)-2-(1H-indol-3-yl)propyl)-4-(4-fluoro-2-methylphenyl)-3-oxo-1-piperazinecarboxamide;
N-((1R,2S)-1-(((5-((dimethylamino)methyl)-2-methoxyphenyl)amino)carbonyl)-2-(1H-indol-3-yl)propyl)-4-(2-methylphenyl)-1-piperazinecarboxamide;
N-((1R,2S)-1-(((5-((dimethylamino)methyl)-2-ethoxyphenyl)amino)carbonyl)-2-(1H-indol-3-yl)propyl)-4-(4-fluorophenyl)-1-piperazinecarboxamide; or
N-((1R,2S)-1-(((5-((dimethylamino)methyl)-2-ethoxyphenyl)amino)carbonyl)-2-(1H-indol-3-yl)propyl)-4-phenyl-1-piperidinecarboxamide.

14. A pharmaceutical preparation comprising the compound according to claim 1, a salt thereof or a prodrug thereof.

15. The pharmaceutical preparation according to claim 14, which is a somatostatin receptor binding inhibitor.

16. The pharmaceutical preparation according to claim 15, which is a somatostatin subtype 2 receptor binding inhibitor.

17. The pharmaceutical preparation according to claim 14, which is a somatostatin receptor agonist.

18. The pharmaceutical preparation according to claim 17, which is a somatostatin subtype 2 receptor agonist.

19. The pharmaceutical preparation according to claim 14, which is a prophylactic or therapeutic agent for diabetes or diabetic complications.

20. The pharmaceutical preparation according to claim 14, which is a prophylactic or therapeutic agent for obesity.

21. (canceled)

22. A method for inhibiting somatostatin receptor binding in a mammal, which comprises administering to the mammal an effective amount of the compound according to claim 1, a salt thereof or a prodrug thereof.

23. (canceled)

24. A method for preventing or treating diabetes or diabetic complications in a mammal, which comprises administering to the mammal an effective amount of the compound according to claim 1, a salt thereof or a prodrug thereof.

25. (canceled)

26. A method for preventing or treating obesity in a mammal, which comprises administering to the mammal an effective amount of the compound according to claim 1, a salt thereof or a prodrug thereof.

27. A method for producing a compound of claim 1 or a salt thereof, which comprises reacting a compound of the formula: 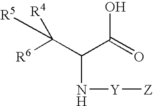
wherein
Y represents a bond or a spacer having a main chain of 1 to 6 atoms;
R⁴ and R⁵ are the same or different, and each represents a hydrogen atom or a hydrocarbon group optionally having substituents, or R⁴ and R⁵, together with the adjacent carbon atom, form a ring optionally having substituents;
R⁶ represents an indolyl group optionally having substituents;
Z represents a hydrogen atom, a halogen atom or a cyclic group optionally having substituents; or a salt thereof, with a compound of the formula: 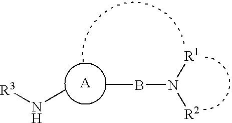
wherein
ring A represents an aromatic ring optionally having substituents;
B represents a bond or a spacer having a main chain of 1 to 6 atoms;
R¹ and R² are the same or different, and each represents a hydrogen atom, a hydrocarbon group optionally having substituents or a heterocyclic group optionally having substituents, or R¹ and R², together with the adjacent nitrogen atom, form a nitrogen-containing heterocyclic ring optionally having substituents, or R¹ is linked with ring A together with the adjacent nitrogen atom and B to form a 5- to 7-membered nitrogen-containing heterocyclic ring;
R³ represents a hydrogen atom, a hydrocarbon group optionally having substituents or a heterocyclic group optionally having substituents; or a salt thereof to give a compound of the formula: 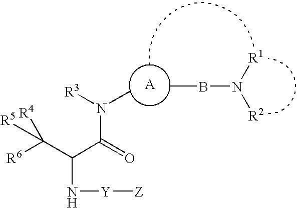
wherein
each symbol is as defined above; or a salt thereof, and optionally reacting the compound or a salt thereof with a compound of the formula: L⁴-Ya-Za wherein L⁴ represents a leaving group; Ya represents a bond or a spacer having a main chain of 1 to 6 atoms; Za represents a hydrogen atom, a halogen atom or a cyclic group optionally having substituents; or a salt thereof.

28. A compound of the formula: 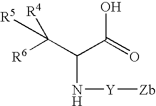
wherein
Y represents a bond or a spacer having a main chain of 1 to 6 atoms;
R⁴ and R⁵ are the same or different, and each represents a hydrogen atom or a hydrocarbon group optionally having substituents, or R⁴ and R⁵, together with the adjacent carbon atom, form a ring optionally having substituents;
R⁶ represents an indolyl group optionally having substituents;
Zb represents piperidinyl or piperazinyl, each of which is substituted by a group of the formula: -Yd-Ara wherein Yd represents a bond or a spacer having a main chain of 1 to 6 atoms, and Ara represents a monocyclic group optionally having substituents; or a salt thereof.
